(19) United States
(12) Patent Application Publication
(10) Pub. No.: US 2022/0098203 A1
(43) Pub. Date: Mar. 31, 2022

(54) AZA-HETEROBICYCLIC INHIBITORS OF MAT2A AND METHODS OF USE FOR TREATING CANCER

(71) Applicant: Les Laboratoires Servier SAS, Suresnes Cedex (FR)

(72) Inventors: Zenon D. KONTEATIS, Chatham, NJ (US); Mingzong LI, Medford, MA (US); Samuel K. REZNIK, Cambridge, MA (US); Zhihua SUI, Suresnes Cedex (FR); Jeremy M. TRAVINS, Southborough, MA (US)

(21) Appl. No.: 17/418,406

(22) PCT Filed: Dec. 27, 2019
(86) PCT No.: PCT/US2019/068653

§ 371 Date: Jun. 25, 2021

Related U.S. Application Data
(60) Provisional application No. 62/785,574, filed on Dec. 27, 2018.

(51) Int. Cl. C07D 487/04; C07D 239/80; C07D 519/00; C07D 403/04; C07D 471/04; A61P 35/00

(52) U.S. Cl. CPC C07D 487/04; C07D 239/80; A61P 35/00; C07D 403/04; C07D 471/04; C07D 519/00

(57) ABSTRACT

The present disclosure provides for compounds according to Formula I, Formula II, and their pharmaceutically acceptable salts, tautomers, and/or isotopologues as described in the disclosure. The compounds are inhibitors of methionine adenosyltransferase isoform 2A (MAT2A). Also provided are pharmaceutical compositions and methods of using the compounds for treating cancers, including some cancers in which the gene encoding methylthioadenosine phosphorylase (MTAP) is deleted.

### CROSS-REFERENCE TO RELATED APPLICATIONS

This application claims the priority of U.S. Provisional Patent Application No. 62/785,574, filed Dec. 27, 2018, the disclosure of which is incorporated herein in its entirety.

### BACKGROUND

Methionine adenosyltransferase (MAT), which is also known as S-adenosylmethionine synthetase, is a cellular enzyme that catalyzes the synthesis of S-adenosyl methionine (SAM or AdoMet) from methionine and ATP; the catalysis is considered to be rate-limiting step of the methionine cycle. SAM is the propylamino donor in polyamine biosynthesis, the principal methyl donor for DNA methylation, and is involved in gene transcription and cellular proliferation as well as the production of secondary metabolites.

Two genes designated as MAT1A and MAT2A encode two distinct catalytic MAT isoforms, respectively. A third gene, MAT2B, encodes a MAT2A regulatory subunit. MAT1A is specifically expressed in the adult liver, whereas MAT2A is widely distributed. Because MAT isoforms differ in catalytic kinetics and regulatory properties, MAT1A-expressing cells have considerably higher SAM levels than do MAT2A-expressing cells. It has been found that hypomethylation of the MAT2A promoter and histone acetylation causes upregulation of MAT2A expression.

In hepatocellular carcinoma (HCC), the downregulation of MAT1A and the up-regulation of MAT2A occur, which is known as the MAT1A:MAT2A switch. The switch, accompanied with up-regulation of MAT2B, results in lower SAM contents, which provide a growth advantage to hepatoma cells. Because MAT2A plays a crucial role in facilitating the growth of hepatoma cells, it is a target for antineoplastic therapy. Recent studies have shown that silencing by using small interfering RNA substantially suppresses growth and induces apoptosis in hepatoma cells. See, e.g., T. Li et al., J Cancer 7(10) (2016) 1317-1327.

Some cancer cell lines that are MTAP deficient are particularly sensitive to inhibition of MAT2A. Marjon et al. (Cell Reports 15(3) (2016) 574-587). MTAP (methylthioadenosine phosphorylase) is an enzyme widely expressed in normal tissues that catalyzes the conversion of methylthioadenosine (MTA) into adenine and 5-methylthioribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine.

MAT2A is dysregulated in additional cancers that lack MTAP-deletion, including hepatocellular carcinoma and leukemia. J. Cai et al., Cancer Res. 58 (1998) 1444-1450; T. S. Jani et al., Cell. Res. 19 (2009) 358-369. Silencing of MAT2A expression via RNA-interference results in anti-proliferative effects in several cancer models. H. Chen et al., Gastroenterology 133 (2007) 207-218; Q. Liu et al. Hepatol. Res. 37 (2007) 376-388.

Many human and murine malignant cells lack MTAP activity. MTAP deficiency is found not only in tissue culture cells but the deficiency is also present in primary leukemias, gliomas, melanomas, pancreatic cancers, non-small cell lung cancers (NSCLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphoma, and mesotheliomas. The gene encoding for human MTAP maps to region 9p21 on human chromosome 9p. This region also contains the tumor suppressor genes p16INK4A (also known as CDKN2A) and pl5INK4B. These genes code for p16 and p15, which are inhibitors of the cyclin D-dependent kinases cdk4 and cdk6, respectively.

The p16INK4A transcript can alternatively be alternative reading frame (ARF) spliced into a transcript encoding pl4ARF. pl4ARF binds to MDM2 and prevents degradation of p53 (Pomerantz et al. (1998) Cell 92:713-723). The 9p21 chromosomal region is of interest because it is frequently homozygously deleted in a variety of cancers, including leukemias, NSLC, pancreatic cancers, gliomas, melanomas, and mesothelioma.

The deletions often inactivate more than one gene. For example, Cairns et al. ((1995) Nat. Gen. 11:210-212) reported that after studying more than 500 primary tumors, almost all the deletions identified in such tumors involved a 170 kb region containing MTAP, pl4ARF and P16INK4A. Carson et al. (WO 99/67634) reported that a correlation exists between the stage of tumor development and loss of homozygosity of the gene encoding MTAP and the gene encoding p16. For example, deletion of the MTAP gene, but not p16INK4A was reported to be indicative of a cancer at an early stage of development, whereas deletion of the genes encoding for p16 and MTAP was reported to be indicative of a cancer at a more advanced stage of tumor development. In some osteosarcoma patients, the MTAP gene was present at diagnosis but was deleted at a later time point (Garcia-Castellano et al., Clin. Cancer Res. 8(3) 2002 782-787).

### SUMMARY

The present disclosure provides compounds that inhibit MAT2A. The compounds and their pharmaceutical compositions are useful in methods for treating various cancers, including those that are refractory to standard treatments, such as surgery, radiation therapy, chemotherapy, and hormonal therapy.

Thus, in accordance with some embodiments, the present disclosure provides a compound according to Formula I or a pharmaceutically acceptable salt, tautomer, and/or isotopologue thereof.

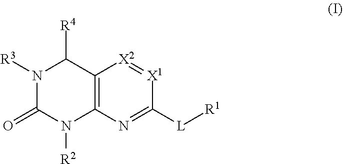

In Formula I, X¹ is N or CR⁵, and X² is N or CR⁶, wherein X¹ and X² are not simultaneously N.

L is O, S, NR, or a bond. Substituent R is H or C₁-C₆-alkyl.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein any alkyl in R¹ is straight or branched.

Further, R¹ is optionally substituted by 1-6 halo. When X¹ is N, X² is CR⁶, L is NR or S, R is H, and R¹ is C₁-C₆-alkyl, then R¹ is substituted by 1-6 halo.

Alternatively, in an embodiment when L is NR, then R and R¹ can be taken together in combination with L to form a 3- to 6-membered heterocycloalkyl (wherein 1-4 ring members are independently selected from N, O, and S) optionally substituted by one or more R^{A}.

R² and R³ are independently selected from the group consisting of optionally substituted C₆-C₁₀-aryl, optionally substituted C₃-C₆-carbocyclyl, optionally substituted 5- to 10-membered heteroaryl (wherein 1-4 heteroaryl members are independently selected from N, O, and S), and optionally substituted 3- to 14-membered heterocycloalkyl (wherein 1-4 heterocycloalkyl members are independently selected from N, O, and S).

R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN.

In other aspects, R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, —NR^{A}C(O)NR^{A}R^{B}, and —CN. In further aspects, R² and/or R³ are —NR^{A}C(O)NR^{A}R^{B}.

R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D}.

R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D}.

R⁶ is selected from the group consisting of H, C₁-C₆-alkyl (optionally substituted by one or more halo), —O(C₁-C₆-alkyl) (optionally substituted by one or more halo), —OH, halo, —CN, —(C₁-C₆-alkyl)NR^{A}R^{B}, and —NR^{A}R^{B}.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S).

In R^{A} and R^{B}, each alkyl, alkoxy, alkenyl, alkynyl, aryl, carbocyclyl, heterocycloalkyl, and heteroaryl moiety is optionally substituted with one or more substituents selected from the group consisting of deuterium, hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl). Each alkyl, alkenyl, aryl, and heterocycloalkyl substituent is optionally substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are each independently selected from H and C₁-C₆-alkyl.

In some aspects, the disclosure is directed to compounds of Formula

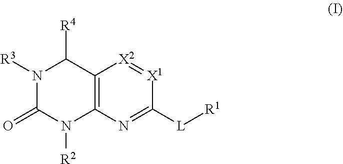

wherein

X¹ is N or CR⁵;

X² is N or CR⁶, wherein X¹ and X² are not simultaneously N;

L is O, S, NR, or a bond;

R is H or C₁-C₆-alkyl;

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein any alkyl in R¹ is straight or branched, R¹ is optionally substituted by 1-6 halo; and when X¹ is N, X² is CR⁶, L is NR or S, R is H, and R¹ is C₁-C₆-alkyl, then R¹ is substituted by 1-6 halo;

or when L is NR, then R and R¹ can be taken together in combination with L to form a 3- to 6-membered heterocycloalkyl (wherein 1-4 ring members are independently selected from N, O, and S) optionally substituted by one or more R^{A};

R² and R³ are independently selected from the group consisting of C₆-C₁₀-aryl, C₃-C₆-carbocyclyl, 5- to 10-membered heteroaryl (wherein 1-4 heteroaryl members are independently selected from N, O, and S), and 3- to 14-membered heterocycloalkyl (wherein 1-4 heterocycloalkyl members are independently selected from N, O, and S), wherein R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN;

R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D};

R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D};

R⁶ is selected from the group consisting of H, C₁-C₆-alkyl (optionally substituted by one or more halo), —O(C₁-C₆-alkyl) (optionally substituted by one or more halo), —OH, halo, —CN, —(C₁-C₆-alkyl)NR^{A}R^{B}, and —NR^{A}R^{B};

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S); wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of deuterium, hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl-C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl), wherein each alkyl, alkenyl, aryl, and heterocycloalkyl is optionally substituted with one or more substituents selected from the group consisting of hydroxy, —OC₁-C₆-alkyl, halo, —NH₂, —(C₁-C₆-alkyl)NH₂, —C(O)OH, CN, and oxo;

R^{C} and R^{D} are each independently selected from H and C₁-C₆-alkyl;

or a pharmaceutically acceptable salt thereof.

In some aspects, the disclosure is directed to compounds of Formula I:

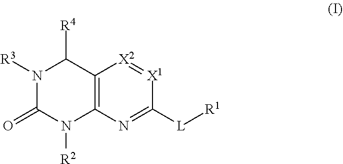

wherein

X¹ is N or CR⁵;

X² is N or CR⁶, wherein X¹ and X² are not simultaneously N;

L is O, S, NR, or a bond;

R is H or C₁-C₆-alkyl;

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein any alkyl in R¹ is straight or branched, R¹ is optionally substituted by 1-6 halo; and when X¹ is N, X² is CR⁶, L is NR or S, R is H, and R¹ is C₁-C₆-alkyl, then R¹ is substituted by 1-6 halo;

or when L is NR, then R and R¹ can be taken together in combination with L to form a 3- to 6-membered heterocycloalkyl (wherein 1-4 ring members are independently selected from N, O, and S) optionally substituted by one or more R^{A};

R² and R³ are independently selected from the group consisting of C₆-C₁₀-aryl, C₃-C₆-carbocyclyl, 5- to 10-membered heteroaryl (wherein 1-4 heteroaryl members are independently selected from N, O, and S), and 3- to 14-membered heterocycloalkyl (wherein 1-4 heterocycloalkyl members are independently selected from N, O, and S), wherein R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, —NR^{A}C(O)NR^{A}R^{B} and —CN;

R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D};

R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D};

R⁶ is selected from the group consisting of H, C₁-C₆-alkyl (optionally substituted by one or more halo), —O(C₁-C₆-alkyl) (optionally substituted by one or more halo), —OH, halo, —CN, —(C₁-C₆-alkyl)NR^{A}R^{B}, and —NR^{A}R^{B};

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S); wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of deuterium, hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl-C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl), wherein each alkyl, alkenyl, aryl, and heterocycloalkyl is optionally substituted with one or more substituents selected from the group consisting of hydroxy, —OC₁-C₆-alkyl, halo, —NH₂, —(C₁-C₆-alkyl)NH₂, —C(O)OH, CN, and oxo;

R^{C} and R^{D} are each independently selected from H and C₁-C₆-alkyl;

or a pharmaceutically acceptable salt thereof.

Another embodiment of the disclosure is a compound according to Formula II, or a pharmaceutically acceptable salt, tautomer, and/or isotopologue thereof:

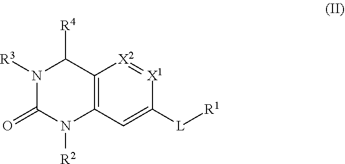

In Formula II, X¹ is N and X² is CR⁶, X¹ is CR⁵ and X² is CR⁶, X¹ and X² are both N, or X¹ is CR⁵ and X² is CR⁶.

L is O, S, NR, or a bond. Substituent R is H or C₁-C₆-alkyl.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein any alkyl in R¹ is straight or branched. R¹ is optionally substituted by 1-6 halo.

In an embodiment when L is NR, then R and R¹ can be taken together in combination with L to form a 3- to 6-membered heterocycloalkyl (wherein 1-4 ring members are independently selected from N, O, and S) optionally substituted by one or more R^{A}.

R² and R³ are independently selected from the group consisting of C₆-C₁₀-aryl, C₃-C₆-carbocyclyl, 5- to 10-membered heteroaryl (wherein 1-4 heteroaryl members are independently selected from N, O, and S), and 3- to 14-membered heterocycloalkyl (wherein 1-4 heterocycloalkyl members are independently selected from N, O, and S). R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN.

R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D}.

R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D}.

R⁶ is selected from the group consisting of H, C₁-C₆-alkyl (optionally substituted by one or more halo), —O(C₁-C₆-alkyl) (optionally substituted by one or more halo), —OH, halo, —CN, —(C₁-C₆-alkyl)NR^{A}R^{B}, and —NR^{A}R^{B}.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S).

Each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl). Each alkyl, alkenyl, aryl, and heterocycloalkyl substituent is optionally substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are each independently selected from H and C₁-C₆-alkyl.

The disclosure provides in another embodiment a pharmaceutical composition comprising a therapeutically effective amount of a compound as described herein or a pharmaceutically acceptable salt, tautomer, and/or isotopologue thereof, and a pharmaceutically acceptable carrier.

In accordance with an additional embodiment, the disclosure provides a method for treating a cancer in a subject suffering therefrom, comprising administering to the subject an effective amount of a MAT2A inhibitor that is a compound, or a pharmaceutically acceptable salt, tautomer, and/or isotopologue as described herein.

The disclosure also provides in a further embodiment a method for inhibiting the synthesis of S-adenosyl methionine (SAM) in a cell, comprising introducing into the cell an effective amount of a compound, or a pharmaceutically acceptable salt, tautomer, and/or isotopologue thereof, as described herein.

The disclosure also provides in a further embodiment a method for inhibiting the synthesis of S-adenosyl methionine (SAM) in a subject, comprising administering to the subject an effective amount of a compound, or a pharmaceutically acceptable salt, tautomer, and/or isotopologue thereof, as described herein.

In another embodiment, the disclosure provides a method for treating a cancer in a subject suffering therefrom, comprising administering to the subject an effective amount of a compound or a pharmaceutically acceptable salt, tautomer, and/or isotopologue thereof, as described herein.

In accordance with still another embodiment, the disclosure provides a method for treating a cancer in a subject suffering therefrom, wherein the cancer is characterized by a reduction or absence of methylthioadenosine phosphorylase (MTAP) gene expression, the absence of the MTAP gene, or reduced function of MTAP protein, as compared to cancers where the MTAP gene or protein is present and/or fully functioning. The method comprises administering to the subject a therapeutically effective amount of a compound, or a pharmaceutically acceptable salt, tautomer, and/or isotopologue thereof, as described herein.

The disclosure provides in an embodiment a compound as described herein, or a pharmaceutically acceptable salt, tautomer, and/or isotopologue thereof, for inhibiting the synthesis of S-adenosyl methionine (SAM).

Another embodiment is a compound as described herein, or a pharmaceutically acceptable salt, tautomer, and/or isotopologue thereof, for treating a cancer in a subject suffering therefrom.

A further embodiment is a compound as described herein, or a pharmaceutically acceptable salt, tautomer, and/or isotopologue thereof, for use in treating a cancer in a subject suffering therefrom.

The disclosure also provides the use of a compound as described herein, or a pharmaceutically acceptable salt thereof, for the manufacture of a medicament for treating cancer.

### DETAILED DESCRIPTION

The compounds described herein are inhibitors of MAT2A. The present disclosure thus relates not only to such compounds in conformity with Formula I or II, but also to their pharmaceutical compositions, tautomers, and/or isotopologues. The compounds and compositions are useful in treating cancers. Some cancers include various MTAP-deleted cancers, i.e., those cancers characterized by the absence or deletion of the MTAP gene or reduced function of the MTAP protein.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein below.

The phrase “substituted alkyl” refers to alkyl substituted at one or more positions, for example, 1, 2, 3, 4, 5, or even 6 positions, which substituents are attached at any available atom to produce a stable compound, with substitution as described herein. “Optionally substituted alkyl” refers to alkyl or substituted alkyl.

Each of the terms “halogen,” “halide,” and “halo” refers to —F, —C₁, —Br, or —I.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein below.

“Substituted alkenyl” refers to alkenyl substituted at 1 or more, e.g., 1, 2, 3, 4, 5, or even 6 positions, which substituents are attached at any available atom to produce a stable compound, with substitution as described herein. “Optionally substituted alkenyl” refers to alkenyl or substituted alkenyl.

“Alkyne or “alkynyl” refers to a straight or branched chain unsaturated hydrocarbon having the indicated number of carbon atoms and at least one triple bond. Examples of a (C₂-C₅)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

“Substituted alkynyl” refers to an alkynyl substituted at 1 or more, e.g., 1, 2, 3, 4, 5, or even 6 positions, which substituents are attached at any available atom to produce a stable compound, with substitution as described herein. “Optionally substituted alkynyl” refers to alkynyl or substituted alkynyl.

The term “alkoxy” refers to an —O-alkyl group having the indicated number of carbon atoms. For example, a (C₁-C₆)alkoxy group includes —O-methyl, —O-ethyl, —O-propyl, —O— isopropyl, —O-butyl, —O-sec-butyl, —O-tert-butyl, —O-pentyl, —O-isopentyl, —O-neopentyl, —O— hexyl, —O-isohexyl, and —O-neohexyl.

The term “carbocyclyl” refers to a monocyclic, bicyclic, tricyclic, or polycyclic, 3- to 14-membered ring system, which is either saturated, such as “cycloalkyl,” or unsaturated, such as “cycloalkenyl.” The term “cycloalkenyl” refers specifically to cyclic alkenyl, such as C₃-C₆-cycloalkenyl. The carbocyclyl may be attached via any atom. Carbocyclyl, for instance, also contemplates fused rings wherein, for instance, a carbocyclyl is fused to an aryl or heteroaryl ring as defined herein. Representative examples of carbocyclyl include, but are not limited to cyclopropyl, cyclobutyl, cyclopentyl, cyclohexyl, cyclopropenyl, cyclobutenyl, cyclopentenyl, cyclohexenyl, phenyl, naphthyl, anthracyl, benzofuranyl, and benzothiophenyl. A carbocyclyl group can be unsubstituted or optionally substituted with one or more substituents as described herein below.

“Substituted carbocyclyl” refers to carbocyclyl substituted at 1 or more, e.g., 1, 2, 3, 4, 5, or even 6 positions, which substituents are attached at any available atom to produce a stable compound, with substitution as described herein. “Optionally substituted carbocyclyl” refers to carbocyclyl or substituted carbocyclyl.

“Aryl” when used alone or as part of another term means a carbocyclic aromatic group whether or not fused having the number of carbon atoms designated or if no number is designated, up to 14 carbon atoms, such as a C₆-C₁₄-aryl. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. “Aryl” also includes aromatic ring systems that are optionally fused with a carbocyclyl ring, as herein defined. An aryl group can be unsubstituted or optionally substituted with one or more substituents as described herein below.

A “substituted aryl” is an aryl that is independently substituted with one or more substituents attached at any available atom to produce a stable compound, wherein the substituents are as described herein. “Optionally substituted aryl” refers to aryl or substituted aryl.

The term “heteroatom” refers to N, O, and S. Inventive compounds that contain N or S atoms can be optionally oxidized to the corresponding N-oxide, sulfoxide, or sulfone compounds.

“Heteroaryl,” alone or in combination with any other moiety described herein, refers to a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein below.

A “substituted heteroaryl” is a heteroaryl that is independently substituted, unless indicated otherwise, with one or more, e.g., 1, 2, 3, 4 or 5, also 1, 2, or 3 substituents, also 1 substituent, attached at any available atom to produce a stable compound, wherein the substituents are as described herein. “Optionally substituted heteroaryl” refers to heteroaryl or substituted heteroaryl.

“Heterocycloalkyl” means a saturated or unsaturated non-aromatic monocyclic, bicyclic, tricyclic or polycyclic ring system that has from 3 to 14, such as 3 to 6, atoms in which from 1 to 3 carbon atoms in the ring are replaced by heteroatoms of O, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein below.

“Optionally substituted heterocycloalkyl” denotes a heterocycloalkyl that is substituted with 1 to 3 substituents, e.g., 1, 2 or 3 substituents, attached at any available atom to produce a stable compound, wherein the substituents are as described herein.

The term “nitrile” or “cyano” can be used interchangeably and refer to a —CN group which is bound to a carbon atom of a heteroaryl ring, aryl ring and a heterocycloalkyl ring.

The term “oxo” refers to a ═O atom attached to a saturated or unsaturated moiety. The ═O atom can be attached to a carbon, sulfur, or nitrogen atom that is part of a cyclic or acyclic moiety.

A “hydroxyl” or “hydroxy” refers to an —OH group.

The substituent —CO₂H may be replaced with bioisosteric replacements such as:

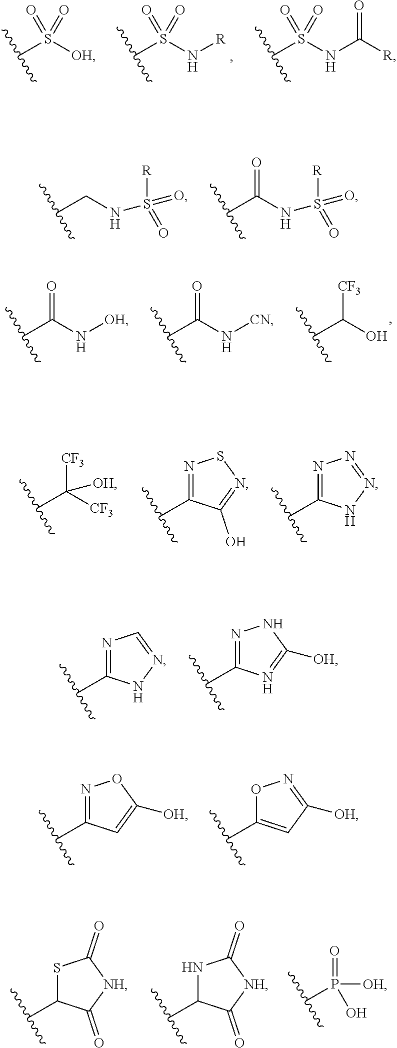

and the like, wherein R has the same definition as R^{A} as defined herein. See, e.g., THE PRACTICE OF MEDICINAL CHEMISTRY (Academic Press: New York, 1996), at page 203.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, the term “isotopologue” is an isotopically enriched compound. As used herein, and unless otherwise indicated, the term “isotopically enriched” refers to an atom having an isotopic composition other than the naturally abundant isotopic composition of that atom. “Isotopically enriched” may also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio between the isotopic composition and the natural isotopic composition of a specified isotope.

With regard to the compounds provided herein, when a particular atom's position is designated as having deuterium or “D” or “²H”, it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound of Formula I or II includes a pharmaceutically acceptable salt of an isotopologue of the compound.

In this description, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or synergies with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

“Inhibitor” means a compound which prevents or reduces the amount of synthesis of SAM. In an embodiment, an inhibitor binds to MAT2A.

### Compounds

As described generally above, the present disclosure provides compounds, pharmaceutically acceptable salts, tautomers, and/or isotopologues thereof, wherein the compounds conform to formula I.

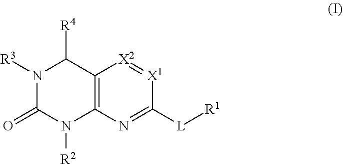

In Formula I, X¹ is N or CR⁵, and X² is N or CR⁶, wherein X¹ and X² are not simultaneously N.

L is O, S, NR, or a bond. Substituent R is H or C₁-C₆-alkyl.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein any alkyl in R¹ is straight or branched.

Further, R¹ is optionally substituted by 1-6 halo. When X¹ is N, X² is CR⁶, L is NR or S, R is H, and R¹ is C₁-C₆-alkyl, then R¹ is substituted by 1-6 halo.

Alternatively, in an embodiment when L is NR, then R and R¹ can be taken together in combination with L to form a 3- to 6-membered heterocycloalkyl (wherein 1-4 ring members are independently selected from N, O, and S) optionally substituted by one or more R^{A}.

R² and R³ are independently selected from the group consisting of optionally substituted C₆-C₁₀-aryl, optionally substituted C₃-C₆-carbocyclyl, optionally substituted 5- to 10-membered heteroaryl (wherein 1-4 heteroaryl members are independently selected from N, O, and S), and optionally substituted 3- to 14-membered heterocycloalkyl (wherein 1-4 heterocycloalkyl members are independently selected from N, O, and S).

R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN. In some embodiments, R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, —NR^{A}C(O)NR^{A}R^{B}, and —CN. In other embodiments, R² and/or R³ are —NR^{A}C(O)NR^{A}R^{B}.

R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D}.

R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D}.

R⁶ is selected from the group consisting of H; C₁-C₆-alkyl optionally substituted by one or more halo; and —O(C₁-C₆-alkyl) optionally substituted by one or more substituents selected from the group consisting of halo, —OH, halo, —CN, —(C₁-C₆-alkyl)NR^{A}R^{B}, and —NR^{A}R^{B}.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S).

In R^{A} and R^{B}, each alkyl, alkoxy, alkenyl, alkynyl, aryl, carbocyclyl, heterocycloalkyl, and heteroaryl moiety is optionally substituted with one or more substituents selected from the group consisting of deuterium, hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl). Each alkyl, alkenyl, aryl, and heterocycloalkyl substituent is optionally substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are each independently selected from H and C₁-C₆-alkyl.

Another embodiment of the disclosure is a compound according to Formula II, or a pharmaceutically acceptable salt thereof:

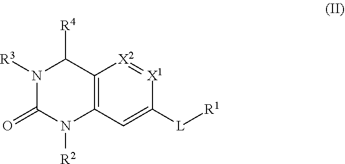

In Formula II, X¹ is N and X² is CR⁶, X¹ is CR⁵ and X² is CR⁶, X¹ and X² are both N, or X¹ is CR⁵ and X² is CR⁶.

L is O, S, NR, or a bond. Substituent R is H or C₁-C₆-alkyl.

R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl), wherein any alkyl in R¹ is straight or branched. R¹ is optionally substituted by 1-6 halo.

In an embodiment when L is NR, then R and R¹ can be taken together in combination with L to form a 3- to 6-membered heterocycloalkyl (wherein 1-4 ring members are independently selected from N, O, and S) optionally substituted by one or more R^{A}.

R² and R³ are independently selected from the group consisting of optionally substituted C₆-C₁₀-aryl, optionally substituted C₃-C₆-carbocyclyl, optionally substituted 5- to 10-membered heteroaryl (wherein 1-4 heteroaryl members are independently selected from N, O, and S), and optionally substituted optionally substituted 3- to 14-membered heterocycloalkyl (wherein 1-4 heterocycloalkyl members are independently selected from N, O, and S). R² and R³ are independently and optionally substituted by one or more substituents that are selected from the group consisting of R^{A}, OR^{A}, halo, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN.

R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D}.

R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D}.

R⁶ is selected from the group consisting of H; C₁-C₆-alkyl optionally substituted by one or more halo; and —O(C₁-C₆-alkyl) optionally substituted by one or more halo, —OH, halo, —CN, —(C₁-C₆-alkyl)NR^{A}R^{B}, and —NR^{A}R^{B}.

R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S).

Each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₁-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl). Each alkyl, alkenyl, aryl, and heterocycloalkyl is optionally substituted with one or more substituents selected from the group consisting of hydroxy, —OC₁-C₆-alkyl, halo, —NH₂, —(C₁-C₆-alkyl)NH₂, —C(O)OH, CN, and oxo.

R^{C} and R^{D} are each independently selected from H and C₁-C₆-alkyl.

In some Formula I compounds, according to an embodiment, X¹ is N and X² is CR⁶. In other embodiments, X¹ is CR⁵ and X² is CR⁶. In still other embodiments, X¹ is CR⁵ and X² is N. Alternatively, X¹ is CR⁵ and X² is CR⁶.

In some Formula II compounds, according to various embodiments, X¹ is N and X² is CR⁶. Other embodiments provide X¹ and X² as both N. In other embodiments, X¹ is CR⁵ and X² is CR⁶.

In combination with any embodiment herein described, per one embodiment, each of R⁴ and R⁵ (when present) is independently selected from H and C₁-C₆-alkyl. In addition, R⁶ (when present) is selected from the group consisting of H, C₁-C₆-alkyl optionally substituted by one or more halo, C₁-C₆-alkoxy, —(C₁-C₆-alkyl)NR^{A}R^{B}, and —NR^{A}R^{B} (wherein R^{A} and R^{B} are independently selected from H and C₁-C₆-alkyl).

In various embodiments, optionally in combination with any other embodiment herein described, at least one of R⁴, R⁵, and R⁶ (when present) is H. Thus, for example, at least R⁴ is H, R⁵ is H, or R⁶ is H. An exemplary compound, in satisfaction of structural requirements described in any embodiment herein, is also one in which each of R⁴, R⁵, and R⁶ (when present) is H.

The disclosure, per another embodiment optionally in combination with any other embodiment, provides for a compound R² is optionally substituted C₆-C₁₀-aryl or optionally substituted 5- to 10-membered heteroaryl. Thus, for example, R² is optionally substituted C₆-C₁₀-aryl, such as optionally substituted phenyl. Alternatively, R² is an optionally substituted 5- to 10-membered heteroaryl, and wherein 1 ring member is N. An example of R² is optionally substituted pyridyl.

A subset of compounds, per various embodiments, is one wherein R³ is optionally substituted 3- to 14-membered heterocycloalkyl or optionally substituted 5- to 10-membered heteroaryl. Examples of R³ include benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl wherein any of the aforementioned moieties is optionally substituted.

In other embodiments, R³ is optionally substituted C₆-C₁₀-aryl. An example of R³ in this context is optionally substituted phenyl.

Some embodiments of the disclosure, optionally in combination with any other embodiment, provide for compounds in which R² is optionally substituted phenyl and R³ is optionally substituted 3- to 14-membered heterocycloalkyl or optionally substituted 5-to 10-membered heteroaryl.

In an embodiment, a compound as described in any other embodiment, is one in which L is O or NR. Optionally in combination with this embodiment, R¹ is optionally substituted C₁-C₆-alkyl or optionally substituted C₃-C₆-carbocyclyl. An exemplary embodiment is one in which R¹ is C₁-C₃-alkyl that is optionally substituted by 1-3 F.

In various embodiments optionally in combination with any other embodiment herein described, L is O or NR and R is H; R¹ is C₁-C₃-alkyl that is optionally substituted by 1-3 F; R² is optionally substituted 3- to 14-membered heterocycloalkyl or optionally substituted 5- to 10-membered heteroaryl (wherein 1 heterocycloalkyl or heteroaryl member is N) or optionally substituted C₆-C₁₀-aryl; R³ is optionally substituted 3- to 14-membered heterocycloalkyl, optionally substituted 5- to 10-membered heteroaryl wherein 1 to 3 heterocycloalkyl or heteroaryl members are independently selected from N, O, and S, or optionally substituted C₆-C₁₀-aryl; and each of R⁴, R⁵, and R⁶ (when present) is H.

For example, L is NR. Alternatively, or in addition, R² is optionally substituted phenyl; and R³ is an optionally substituted 5- to 10-membered heteroaryl wherein 1 to 3 heteroaryl members are independently selected from N, O, and S. For instance, R³ is selected from the group consisting of optionally substituted benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl, any of which may be optionally substituted.

In other embodiments, R² is an optionally substituted 5- to 10-membered heteroaryl wherein 1 to 3 heteroaryl members are independently selected from N, O, and S; and R³ is optionally substituted phenyl. In still other embodiments, R² and R³ independently are optionally substituted phenyl.

In various embodiments, the disclosure provides specific examples of Formula I and Formula II compounds, and their pharmaceutically acceptable salts, tautomers, and/or isotopologues thereof as set forth in Table 1 and Table 2 below, respectively, and in Table 3 and Table 4.

TABLE 1
Examples of Formula I Compounds

| | |
|---|---|
| 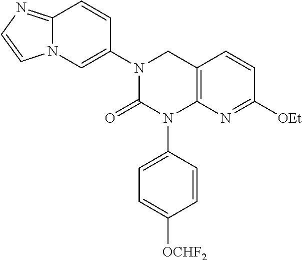 [SMILES: CCOc1ccc2c(n1)N(c1ccc(OC(F)F)cc1)C(=O)N(c1ccc3nccn3c1)C2] | 101 |
| 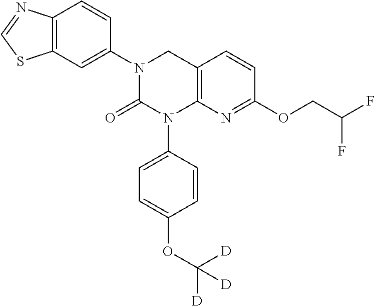 [SMILES: [2H]C([2H])([2H])Oc1ccc(N2C(=O)N(c3ccc4ncsc4c3)Cc3ccc(OCC(F)F)nc32)cc1] | 102 |
| 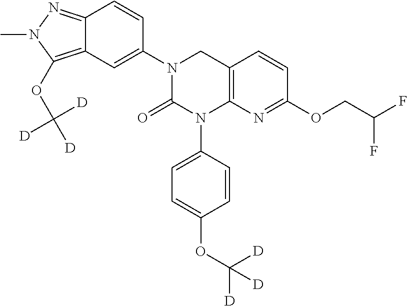 [SMILES: [2H]C([2H])([2H])Oc1ccc(N2C(=O)N(c3ccc4nn(C)c(OC([2H])([2H])[2H])c4c3)Cc3ccc(OCC(F)F)nc32)cc1] | 103 |
| 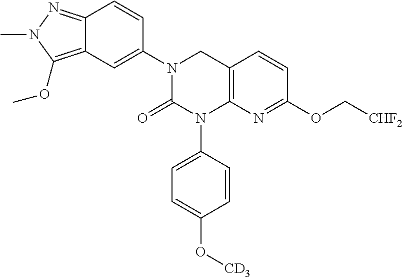 [SMILES: [2H]C([2H])([2H])Oc1ccc(N2C(=O)N(c3ccc4nn(C)c(OC)c4c3)Cc3ccc(OCC(F)F)nc32)cc1] | 104 |
| 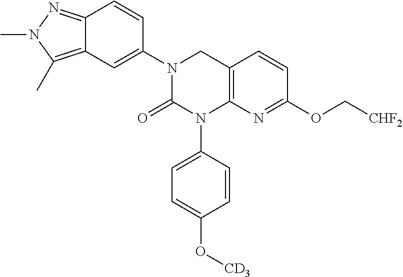 [SMILES: [2H]C([2H])([2H])Oc1ccc(N2C(=O)N(c3ccc4nn(C)c(C)c4c3)Cc3ccc(OCC(F)F)nc32)cc1] | 105 |
| 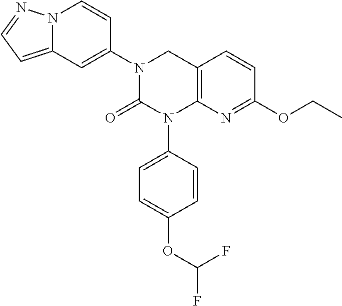 [SMILES: CCOc1ccc2c(n1)N(c1ccc(OC(F)F)cc1)C(=O)N(c1ccn3nccc3c1)C2] | 106 |
| 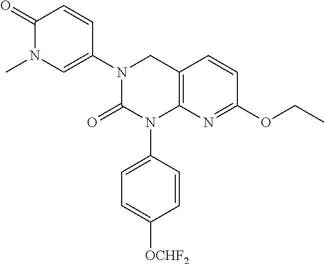 [SMILES: CCOc1ccc2c(n1)N(c1ccc(OC(F)F)cc1)C(=O)N(c1ccc(=O)n(C)c1)C2] | 107 |
| 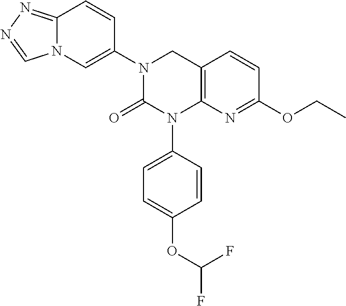 [SMILES: CCOc1ccc2c(n1)N(c1ccc(OC(F)F)cc1)C(=O)N(c1ccc3nncn3c1)C2] | 108 |
| 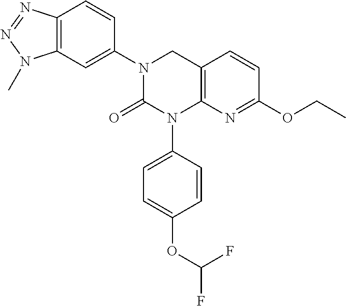 [SMILES: CCOc1ccc2c(n1)N(c1ccc(OC(F)F)cc1)C(=O)N(c1ccc3nnn(C)c3c1)C2] | 109 |
| 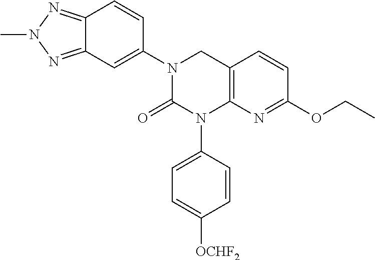 [SMILES: CCOc1ccc2c(n1)N(c1ccc(OC(F)F)cc1)C(=O)N(c1ccc3nn(C)nc3c1)C2] | 110 |
| 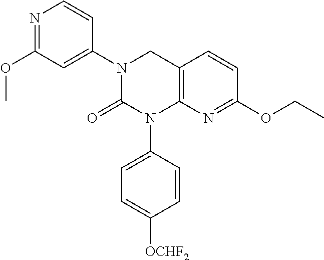 [SMILES: CCOc1ccc2c(n1)N(c1ccc(OC(F)F)cc1)C(=O)N(c1ccnc(OC)c1)C2] | 111 |
| 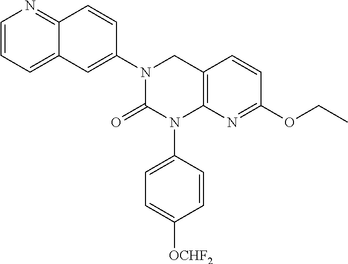 [SMILES: CCOc1ccc2c(n1)N(c1ccc(OC(F)F)cc1)C(=O)N(c1ccc3ncccc3c1)C2] | 112 |
| 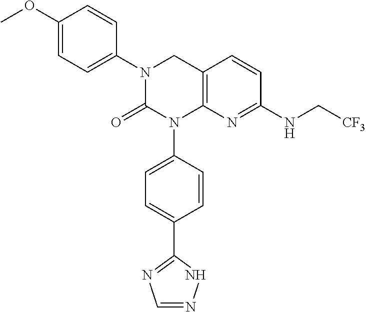 [SMILES: COc1ccc(N2Cc3ccc(NCC(F)(F)F)nc3N(c3ccc(-c4ncn[nH]4)cc3)C2=O)cc1] | 113 |
| 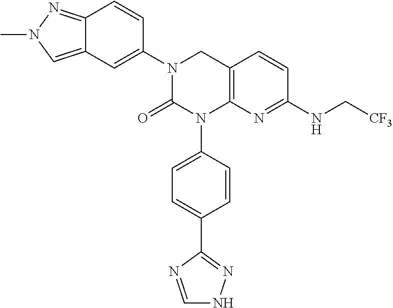 [SMILES: Cn1cc2cc(N3Cc4ccc(NCC(F)(F)F)nc4N(c4ccc(-c5nc[nH]n5)cc4)C3=O)ccc2n1] | 114 |
| 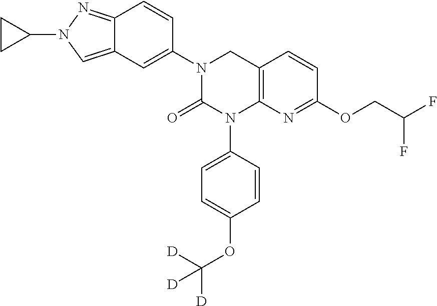 [SMILES: [2H]C([2H])([2H])Oc1ccc(N2C(=O)N(c3ccc4nn(C5CC5)cc4c3)Cc3ccc(OCC(F)F)nc32)cc1] | 115 |
| 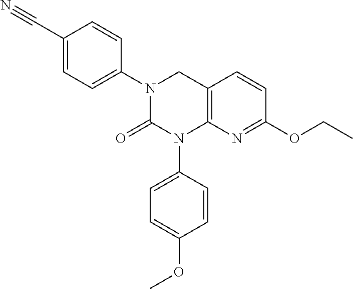 [SMILES: CCOc1ccc2c(n1)N(c1ccc(OC)cc1)C(=O)N(c1ccc(C#N)cc1)C2] | 116 |
| 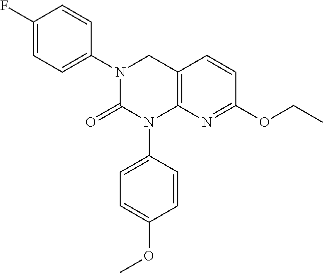 [SMILES: CCOc1ccc2c(n1)N(c1ccc(OC)cc1)C(=O)N(c1ccc(F)cc1)C2] | 117 |
| 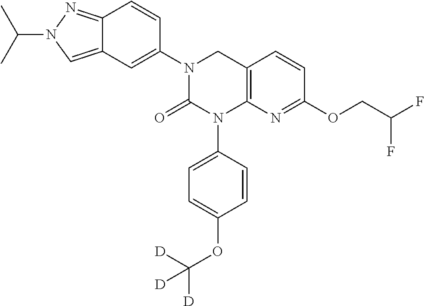 [SMILES: [2H]C([2H])([2H])Oc1ccc(N2C(=O)N(c3ccc4nn(C(C)C)cc4c3)Cc3ccc(OCC(F)F)nc32)cc1] | 118 |
| 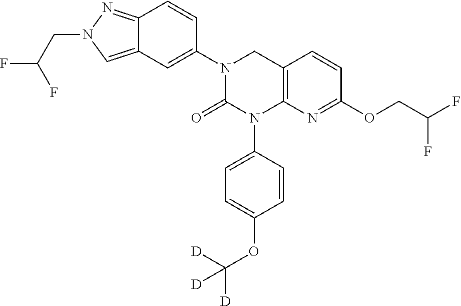 [SMILES: [2H]C([2H])([2H])Oc1ccc(N2C(=O)N(c3ccc4nn(CC(F)F)cc4c3)Cc3ccc(OCC(F)F)nc32)cc1] | 119 |
| 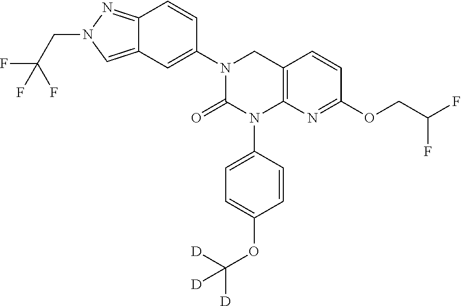 [SMILES: [2H]C([2H])([2H])Oc1ccc(N2C(=O)N(c3ccc4nn(CC(F)(F)F)cc4c3)Cc3ccc(OCC(F)F)nc32)cc1] | 120 |
| 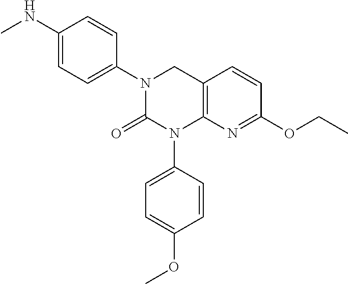 [SMILES: CCOc1ccc2c(n1)N(c1ccc(OC)cc1)C(=O)N(c1ccc(NC)cc1)C2] | 121 |
| 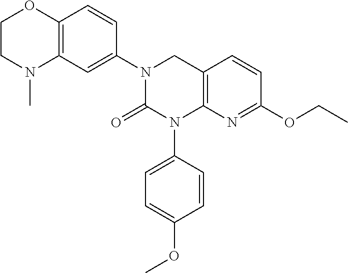 [SMILES: CCOc1ccc2c(n1)N(c1ccc(OC)cc1)C(=O)N(c1ccc3c(c1)N(C)CCO3)C2] | 122 |
| 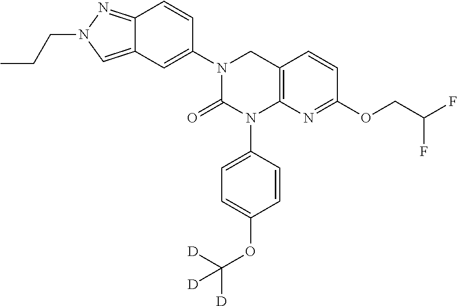 [SMILES: [2H]C([2H])([2H])Oc1ccc(N2C(=O)N(c3ccc4nn(CCC)cc4c3)Cc3ccc(OCC(F)F)nc32)cc1] | 123 |
| 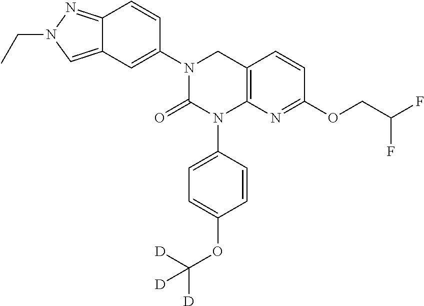 [SMILES: [2H]C([2H])([2H])Oc1ccc(N2C(=O)N(c3ccc4nn(CC)cc4c3)Cc3ccc(OCC(F)F)nc32)cc1] | 124 |
| 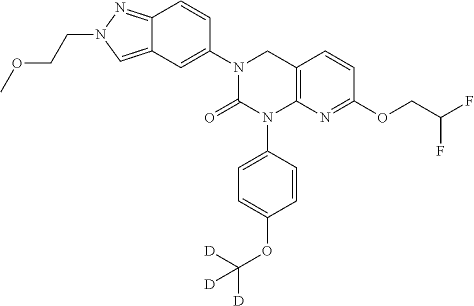 [SMILES: [2H]C([2H])([2H])Oc1ccc(N2C(=O)N(c3ccc4nn(CCOC)cc4c3)Cc3ccc(OCC(F)F)nc32)cc1] | 125 |
| 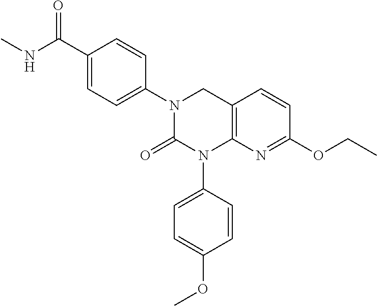 [SMILES: CCOc1ccc2c(n1)N(c1ccc(OC)cc1)C(=O)N(c1ccc(C(=O)NC)cc1)C2] | 126 |
| 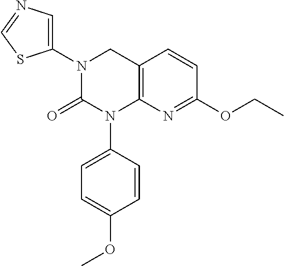 [SMILES: CCOc1ccc2c(n1)N(c1ccc(OC)cc1)C(=O)N(c1cncs1)C2] | 127 |
| 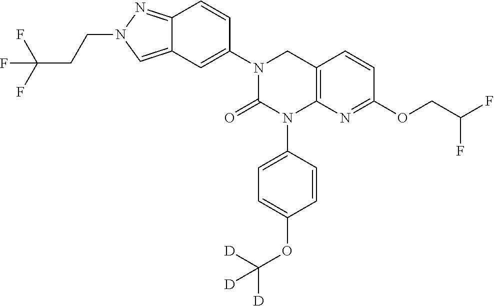 [SMILES: [2H]C([2H])([2H])Oc1ccc(N2C(=O)N(c3ccc4nn(CCC(F)(F)F)cc4c3)Cc3ccc(OCC(F)F)nc32)cc1] | 128 |
| 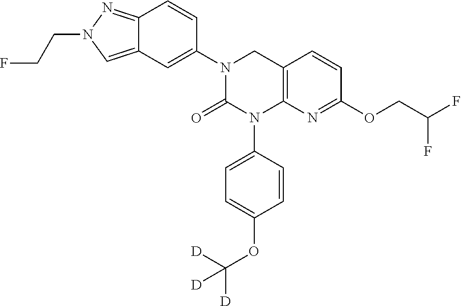 [SMILES: [2H]C([2H])([2H])Oc1ccc(N2C(=O)N(c3ccc4nn(CCF)cc4c3)Cc3ccc(OCC(F)F)nc32)cc1] | 129 |
| 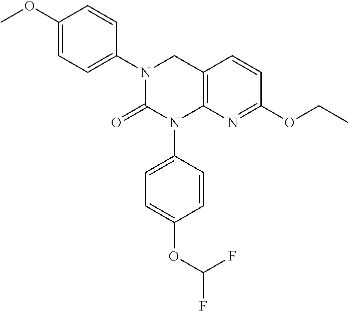 [SMILES: CCOc1ccc2c(n1)N(c1ccc(OC(F)F)cc1)C(=O)N(c1ccc(OC)cc1)C2] | 130 |
| 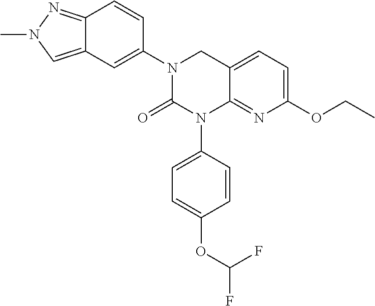 [SMILES: CCOc1ccc2c(n1)N(c1ccc(OC(F)F)cc1)C(=O)N(c1ccc3nn(C)cc3c1)C2] | 131 |
| 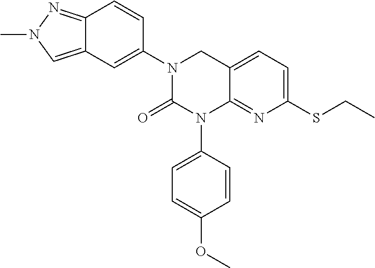 [SMILES: CCSc1ccc2c(n1)N(c1ccc(OC)cc1)C(=O)N(c1ccc3nn(C)cc3c1)C2] | 132 |
| 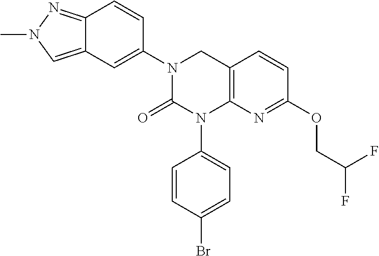 [SMILES: Cn1cc2cc(N3Cc4ccc(OCC(F)F)nc4N(c4ccc(Br)cc4)C3=O)ccc2n1] | 133 |
| 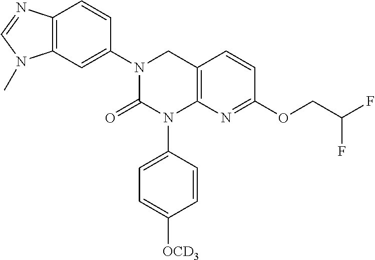 | 135 |
| 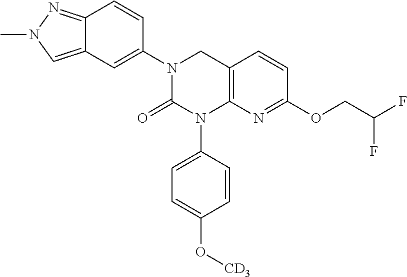 [SMILES: [2H]C([2H])([2H])Oc1ccc(N2C(=O)N(c3ccc4nn(C)cc4c3)Cc3ccc(OCC(F)F)nc32)cc1] | 136 |
| 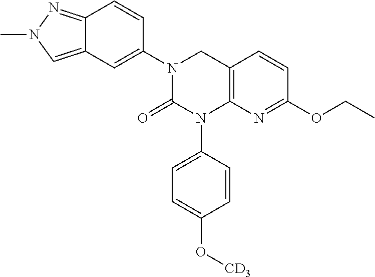 [SMILES: [2H]C([2H])([2H])Oc1ccc(N2C(=O)N(c3ccc4nn(C)cc4c3)Cc3ccc(OCC)nc32)cc1] | 137 |
| 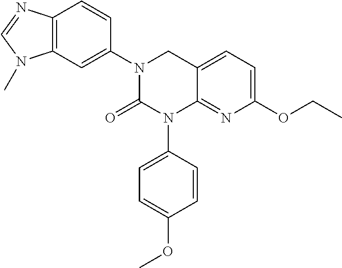 [SMILES: CCOc1ccc2c(n1)N(c1ccc(OC)cc1)C(=O)N(c1ccc3ncn(C)c3c1)C2] | 138 |
| 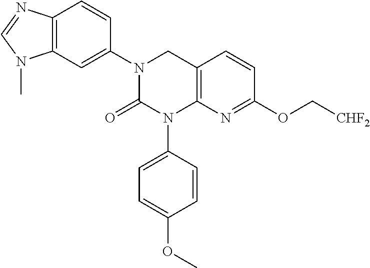 [SMILES: COc1ccc(N2C(=O)N(c3ccc4ncn(C)c4c3)Cc3ccc(OCC(F)F)nc32)cc1] | 139 |
| 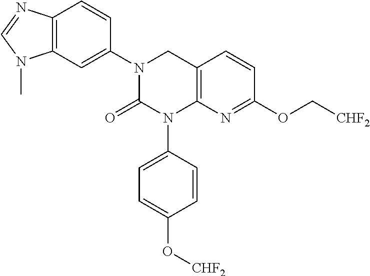 [SMILES: Cn1cnc2ccc(N3Cc4ccc(OCC(F)F)nc4N(c4ccc(OC(F)F)cc4)C3=O)cc21] | 140 |
| 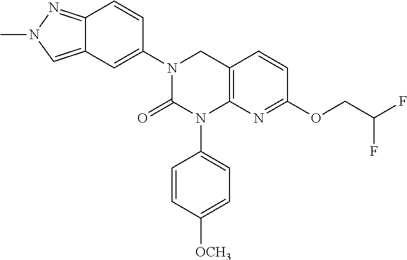 [SMILES: COc1ccc(N2C(=O)N(c3ccc4nn(C)cc4c3)Cc3ccc(OCC(F)F)nc32)cc1] | 141 |
| 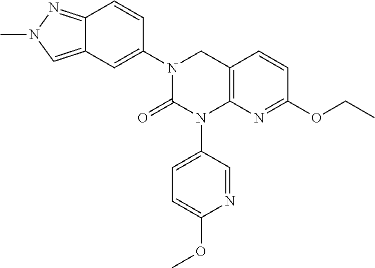 [SMILES: CCOc1ccc2c(n1)N(c1ccc(OC)nc1)C(=O)N(c1ccc3nn(C)cc3c1)C2] | 142 |
| 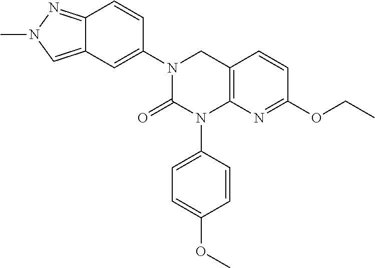 [SMILES: CCOc1ccc2c(n1)N(c1ccc(OC)cc1)C(=O)N(c1ccc3nn(C)cc3c1)C2] | 143 |
| 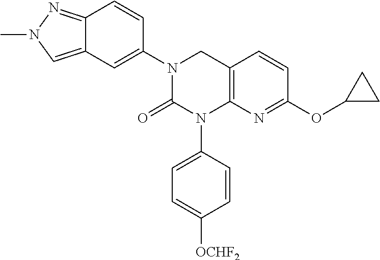 [SMILES: Cn1cc2cc(N3Cc4ccc(OC5CC5)nc4N(c4ccc(OC(F)F)cc4)C3=O)ccc2n1] | 144 |
| 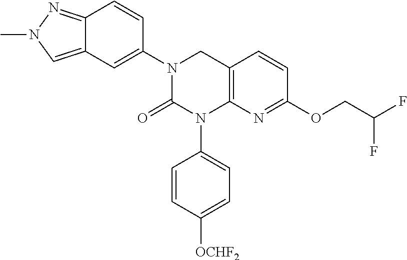 [SMILES: Cn1cc2cc(N3Cc4ccc(OCC(F)F)nc4N(c4ccc(OC(F)F)cc4)C3=O)ccc2n1] | 145 |
| 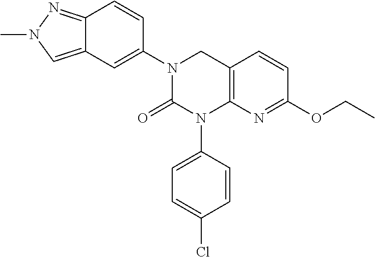 [SMILES: CCOc1ccc2c(n1)N(c1ccc(Cl)cc1)C(=O)N(c1ccc3nn(C)cc3c1)C2] | 146 |
| 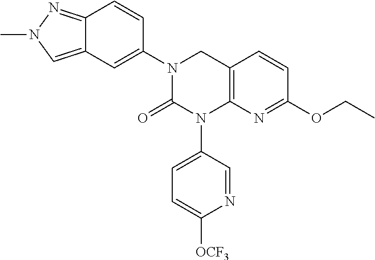 [SMILES: CCOc1ccc2c(n1)N(c1ccc(OC(F)(F)F)nc1)C(=O)N(c1ccc3nn(C)cc3c1)C2] | 147 |
| 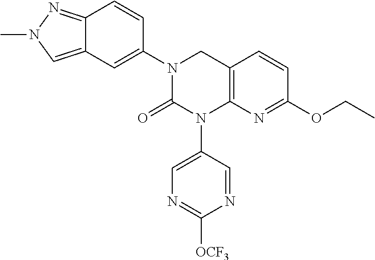 [SMILES: CCOc1ccc2c(n1)N(c1cnc(OC(F)(F)F)nc1)C(=O)N(c1ccc3nn(C)cc3c1)C2] | 148 |
| 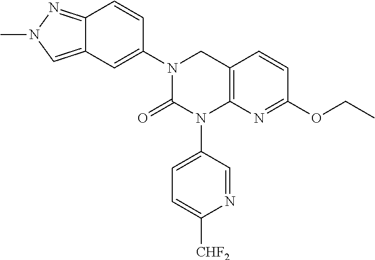 [SMILES: CCOc1ccc2c(n1)N(c1ccc(C(F)F)nc1)C(=O)N(c1ccc3nn(C)cc3c1)C2] | 149 |
| 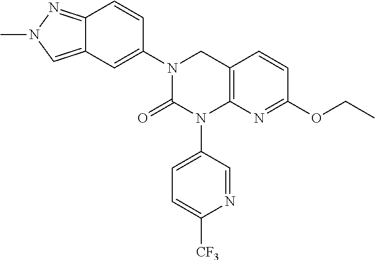 [SMILES: CCOc1ccc2c(n1)N(c1ccc(C(F)(F)F)nc1)C(=O)N(c1ccc3nn(C)cc3c1)C2] | 150 |
| 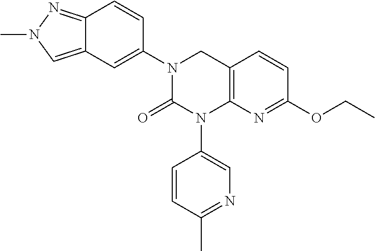 [SMILES: CCOc1ccc2c(n1)N(c1ccc(C)nc1)C(=O)N(c1ccc3nn(C)cc3c1)C2] | 151 |
| 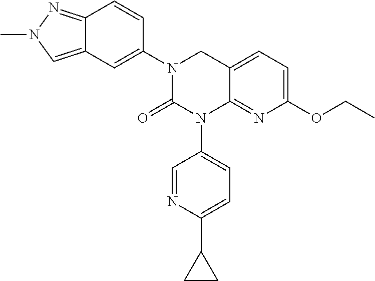 [SMILES: CCOc1ccc2c(n1)N(c1ccc(C3CC3)nc1)C(=O)N(c1ccc3nn(C)cc3c1)C2] | 152 |
| 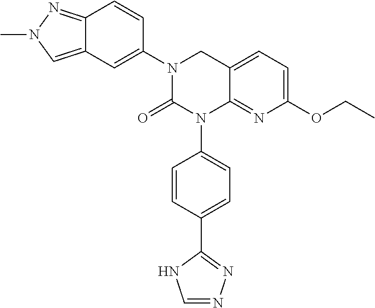 [SMILES: CCOc1ccc2c(n1)N(c1ccc(-c3nnc[nH]3)cc1)C(=O)N(c1ccc3nn(C)cc3c1)C2] | 153 |
| 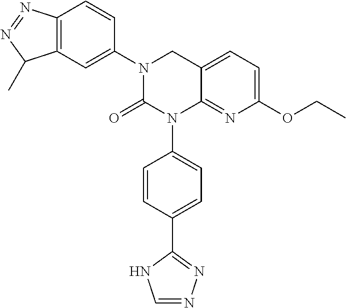 [SMILES: CCOc1ccc2c(n1)N(c1ccc(-c3nnc[nH]3)cc1)C(=O)N(c1ccc3c(c1)C(C)N=N3)C2] | 154 |
| 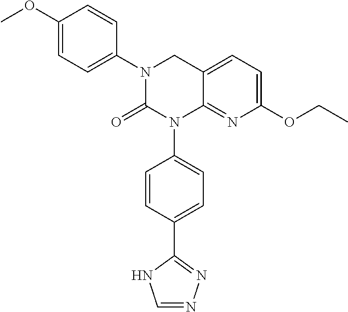 [SMILES: CCOc1ccc2c(n1)N(c1ccc(-c3nnc[nH]3)cc1)C(=O)N(c1ccc(OC)cc1)C2] | 155 |
| 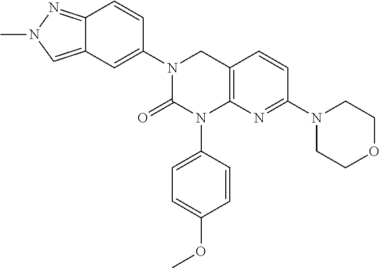 [SMILES: COc1ccc(N2C(=O)N(c3ccc4nn(C)cc4c3)Cc3ccc(N4CCOCC4)nc32)cc1] | 157 |
| 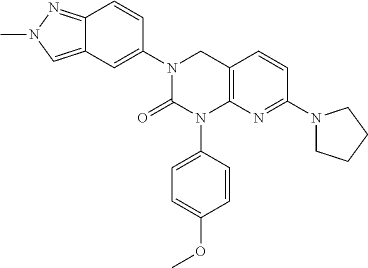 [SMILES: COc1ccc(N2C(=O)N(c3ccc4nn(C)cc4c3)Cc3ccc(N4CCCC4)nc32)cc1] | 158 |
| 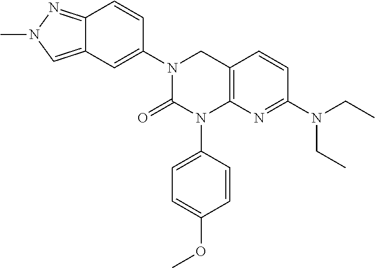 [SMILES: CCN(CC)c1ccc2c(n1)N(c1ccc(OC)cc1)C(=O)N(c1ccc3nn(C)cc3c1)C2] | 159 |
| 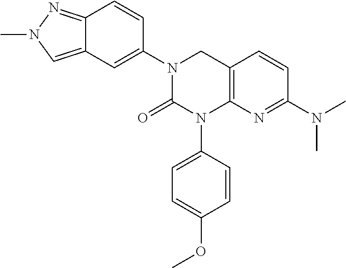 | 160 |
| 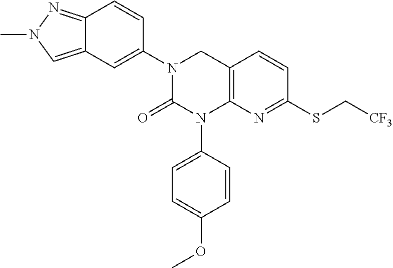 [SMILES: COc1ccc(N2C(=O)N(c3ccc4nn(C)cc4c3)Cc3ccc(SCC(F)(F)F)nc32)cc1] | 161 |
| 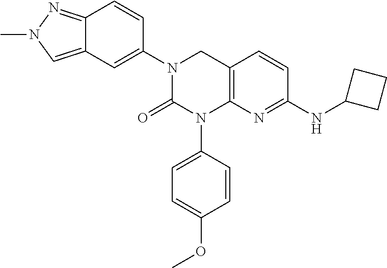 [SMILES: COc1ccc(N2C(=O)N(c3ccc4nn(C)cc4c3)Cc3ccc(NC4CCC4)nc32)cc1] | 162 |
| 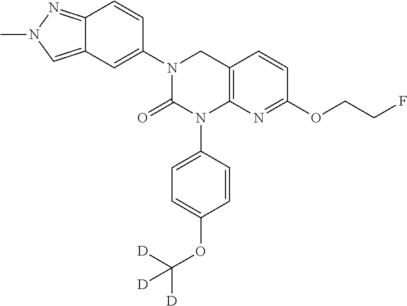 [SMILES: [2H]C([2H])([2H])Oc1ccc(N2C(=O)N(c3ccc4nn(C)cc4c3)Cc3ccc(OCCF)nc32)cc1] | 163 |
| 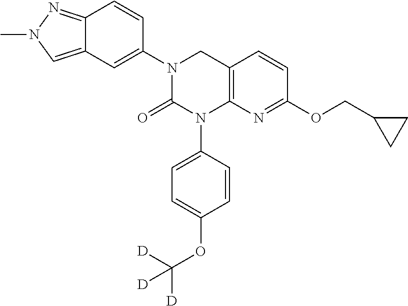 [SMILES: [2H]C([2H])([2H])Oc1ccc(N2C(=O)N(c3ccc4nn(C)cc4c3)Cc3ccc(OCC4CC4)nc32)cc1] | 164 |
| 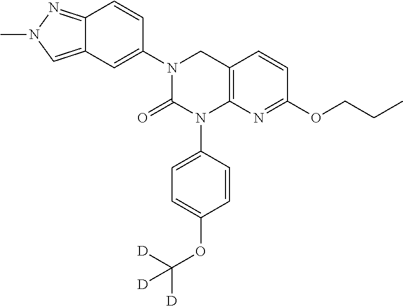 [SMILES: [2H]C([2H])([2H])Oc1ccc(N2C(=O)N(c3ccc4nn(C)cc4c3)Cc3ccc(OCCC)nc32)cc1] | 165 |
| 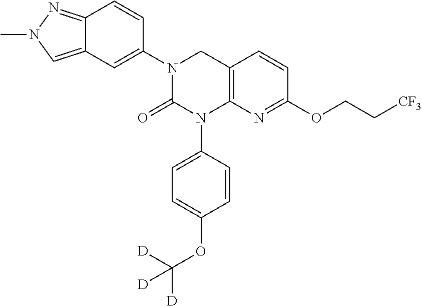 [SMILES: [2H]C([2H])([2H])Oc1ccc(N2C(=O)N(c3ccc4nn(C)cc4c3)Cc3ccc(OCCC(F)(F)F)nc32)cc1] | 166 |
| 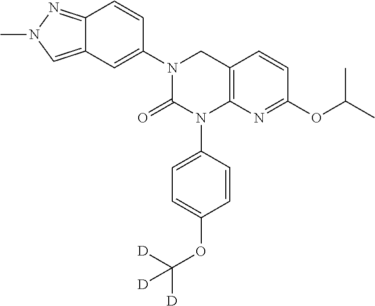 [SMILES: [2H]C([2H])([2H])Oc1ccc(N2C(=O)N(c3ccc4nn(C)cc4c3)Cc3ccc(OC(C)C)nc32)cc1] | 167 |
| 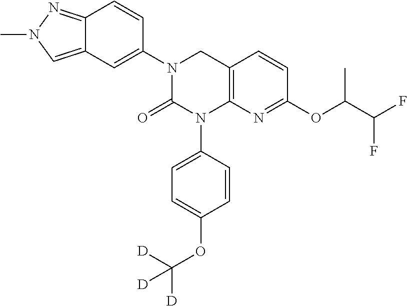 [SMILES: [2H]C([2H])([2H])Oc1ccc(N2C(=O)N(c3ccc4nn(C)cc4c3)Cc3ccc(OC(C)C(F)F)nc32)cc1] | 168 |
| 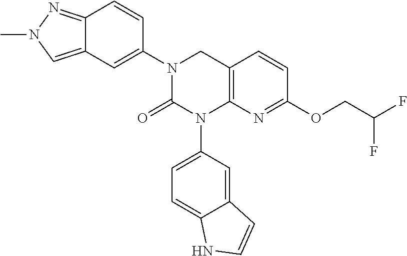 [SMILES: Cn1cc2cc(N3Cc4ccc(OCC(F)F)nc4N(c4ccc5[nH]ccc5c4)C3=O)ccc2n1] | 169 |
| 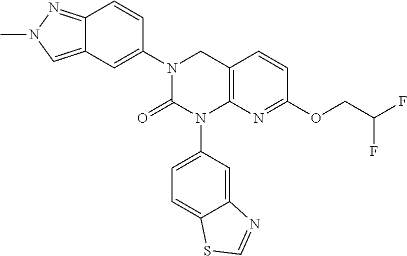 [SMILES: Cn1cc2cc(N3Cc4ccc(OCC(F)F)nc4N(c4ccc5scnc5c4)C3=O)ccc2n1] | 170 |
| 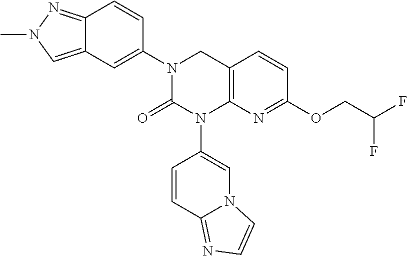 [SMILES: Cn1cc2cc(N3Cc4ccc(OCC(F)F)nc4N(c4ccc5nccn5c4)C3=O)ccc2n1] | 171 |
| 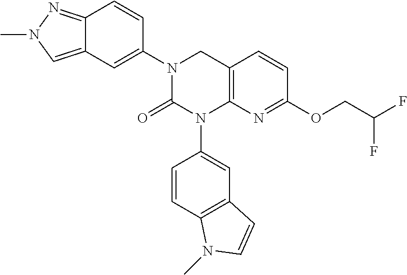 [SMILES: Cn1cc2cc(N3Cc4ccc(OCC(F)F)nc4N(c4ccc5c(ccn5C)c4)C3=O)ccc2n1] | 172 |
| 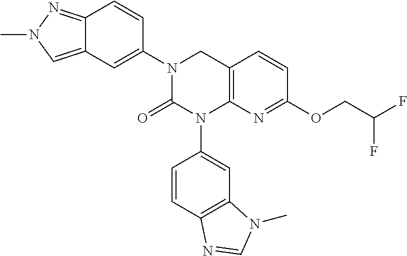 [SMILES: Cn1cc2cc(N3Cc4ccc(OCC(F)F)nc4N(c4ccc5ncn(C)c5c4)C3=O)ccc2n1] | 173 |
| 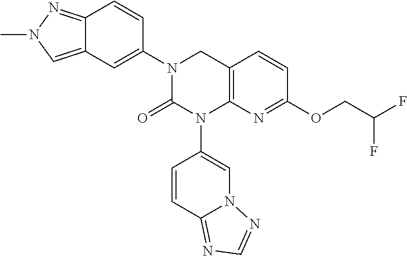 [SMILES: Cn1cc2cc(N3Cc4ccc(OCC(F)F)nc4N(c4ccc5ncnn5c4)C3=O)ccc2n1] | 174 |
| 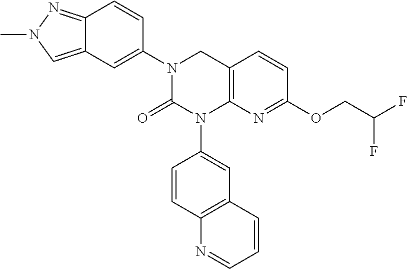 [SMILES: Cn1cc2cc(N3Cc4ccc(OCC(F)F)nc4N(c4ccc5ncccc5c4)C3=O)ccc2n1] | 175 |
| 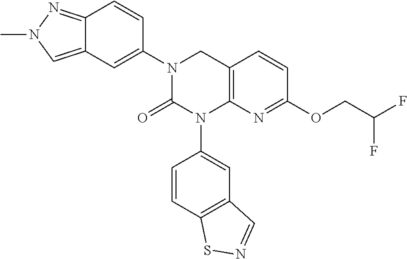 [SMILES: Cn1cc2cc(N3Cc4ccc(OCC(F)F)nc4N(c4ccc5sncc5c4)C3=O)ccc2n1] | 176 |
| 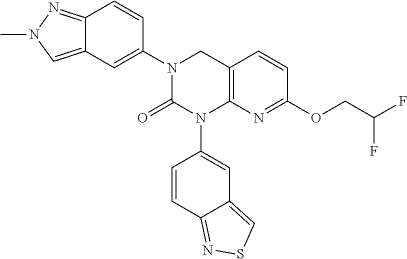 [SMILES: Cn1cc2cc(N3Cc4ccc(OCC(F)F)nc4N(c4ccc5nscc5c4)C3=O)ccc2n1] | 177 |
| 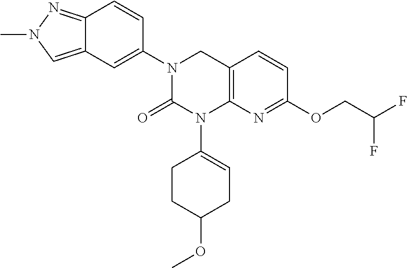 [SMILES: COC1CC=C(N2C(=O)N(c3ccc4nn(C)cc4c3)Cc3ccc(OCC(F)F)nc32)CC1] | 178 |
| 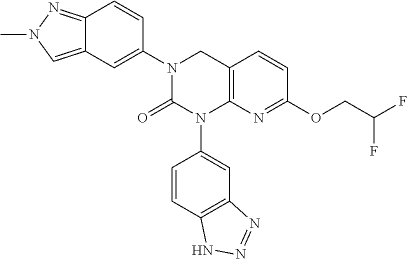 [SMILES: Cn1cc2cc(N3Cc4ccc(OCC(F)F)nc4N(c4ccc5[nH]nnc5c4)C3=O)ccc2n1] | 179 |
| 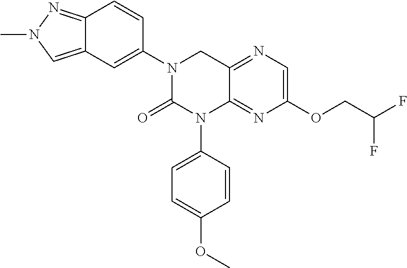 [SMILES: COc1ccc(N2C(=O)N(c3ccc4nn(C)cc4c3)Cc3ncc(OCC(F)F)nc32)cc1] | 180 |
| 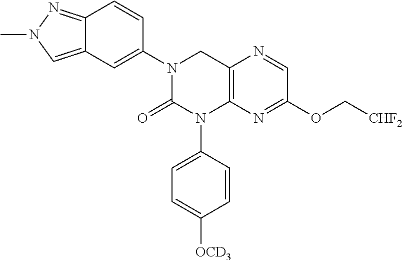 | 181 |
| 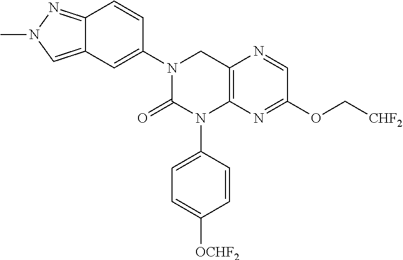 [SMILES: Cn1cc2cc(N3Cc4ncc(OCC(F)F)nc4N(c4ccc(OC(F)F)cc4)C3=O)ccc2n1] | 182 |
| 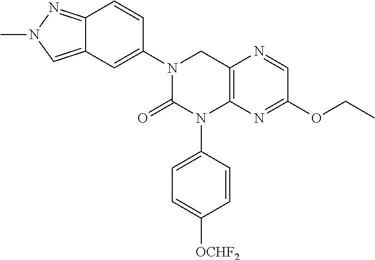 [SMILES: CCOc1cnc2c(n1)N(c1ccc(OC(F)F)cc1)C(=O)N(c1ccc3nn(C)cc3c1)C2] | 183 |
| 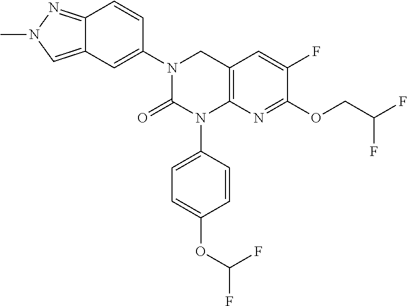 [SMILES: Cn1cc2cc(N3Cc4cc(F)c(OCC(F)F)nc4N(c4ccc(OC(F)F)cc4)C3=O)ccc2n1] | 184 |
| 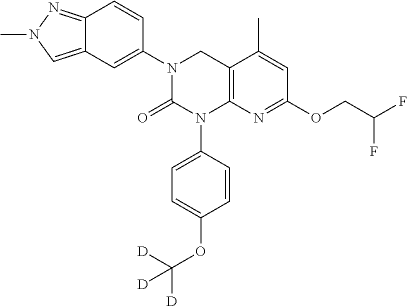 [SMILES: [2H]C([2H])([2H])Oc1ccc(N2C(=O)N(c3ccc4nn(C)cc4c3)Cc3c(C)cc(OCC(F)F)nc32)cc1] | 185 |
| 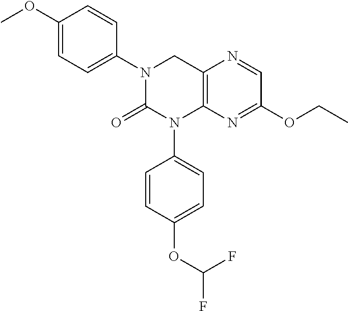 [SMILES: CCOc1cnc2c(n1)N(c1ccc(OC(F)F)cc1)C(=O)N(c1ccc(OC)cc1)C2] | 186 |
| 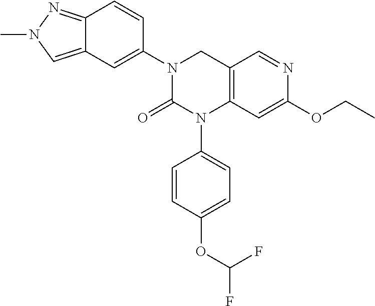 [SMILES: CCOc1cc2c(cn1)CN(c1ccc3nn(C)cc3c1)C(=O)N2c1ccc(OC(F)F)cc1] | 187 |
| 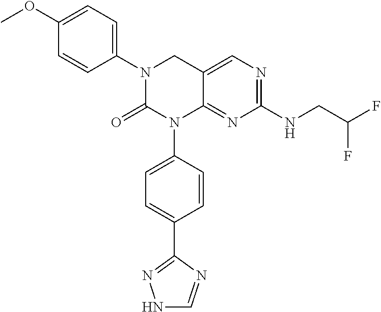 [SMILES: COc1ccc(N2Cc3cnc(NCC(F)F)nc3N(c3ccc(-c4nc[nH]n4)cc3)C2=O)cc1] | 188 |
| 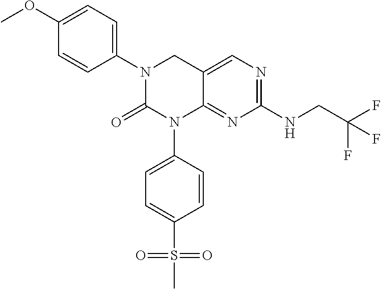 [SMILES: COc1ccc(N2Cc3cnc(NCC(F)(F)F)nc3N(c3ccc(S(C)(=O)=O)cc3)C2=O)cc1] | 189 |
| 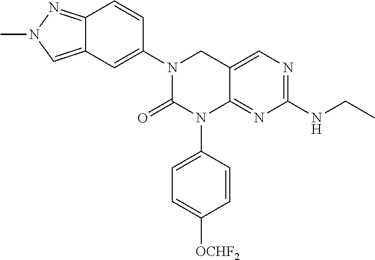 [SMILES: CCNc1ncc2c(n1)N(c1ccc(OC(F)F)cc1)C(=O)N(c1ccc3nn(C)cc3c1)C2] | 190 |
| 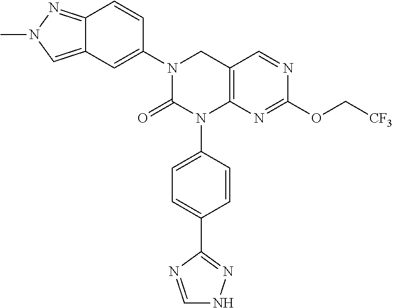 [SMILES: Cn1cc2cc(N3Cc4cnc(OCC(F)(F)F)nc4N(c4ccc(-c5nc[nH]n5)cc4)C3=O)ccc2n1] | 191 |
| 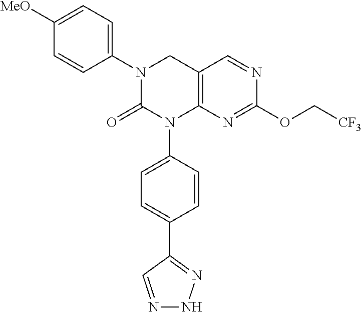 [SMILES: COc1ccc(N2Cc3cnc(OCC(F)(F)F)nc3N(c3ccc(-c4cn[nH]n4)cc3)C2=O)cc1] | 192 |
| 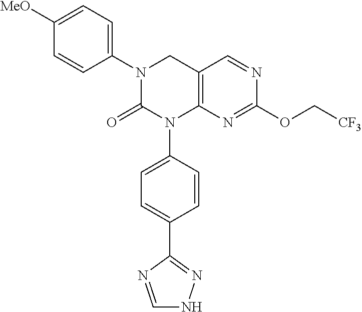 [SMILES: COc1ccc(N2Cc3cnc(OCC(F)(F)F)nc3N(c3ccc(-c4nc[nH]n4)cc3)C2=O)cc1] | 193 |
| 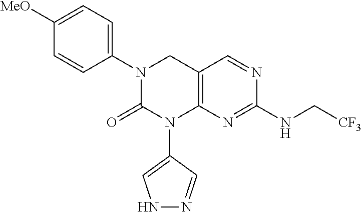 [SMILES: COc1ccc(N2Cc3cnc(NCC(F)(F)F)nc3N(c3cn[nH]c3)C2=O)cc1] | 194 |
| 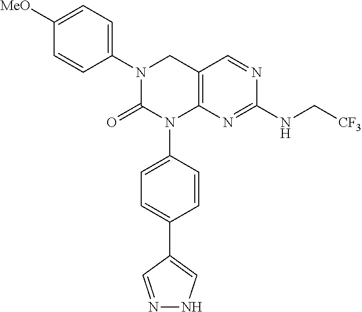 [SMILES: COc1ccc(N2Cc3cnc(NCC(F)(F)F)nc3N(c3ccc(-c4cn[nH]c4)cc3)C2=O)cc1] | 195 |
| 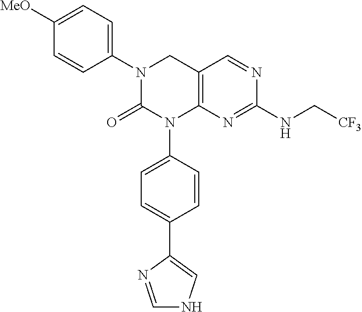 [SMILES: COc1ccc(N2Cc3cnc(NCC(F)(F)F)nc3N(c3ccc(-c4c[nH]cn4)cc3)C2=O)cc1] | 196 |
| 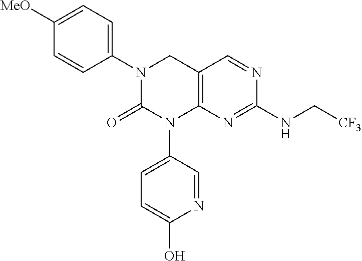 [SMILES: COc1ccc(N2Cc3cnc(NCC(F)(F)F)nc3N(c3ccc(O)nc3)C2=O)cc1] | 197 |
| 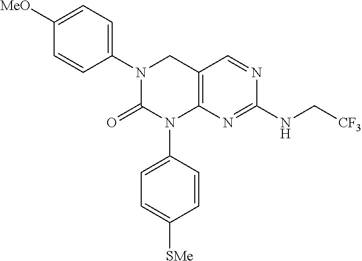 [SMILES: COc1ccc(N2Cc3cnc(NCC(F)(F)F)nc3N(c3ccc(SC)cc3)C2=O)cc1] | 198 |
| 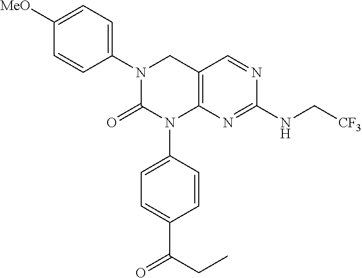 [SMILES: CCC(=O)c1ccc(N2C(=O)N(c3ccc(OC)cc3)Cc3cnc(NCC(F)(F)F)nc32)cc1] | 199 |
| 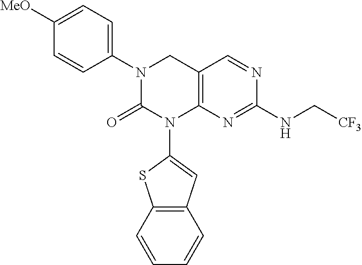 [SMILES: COc1ccc(N2Cc3cnc(NCC(F)(F)F)nc3N(c3cc4ccccc4s3)C2=O)cc1] | 200 |
| 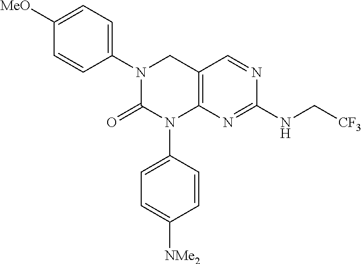 [SMILES: COc1ccc(N2Cc3cnc(NCC(F)(F)F)nc3N(c3ccc(N(C)C)cc3)C2=O)cc1] | 201 |
| 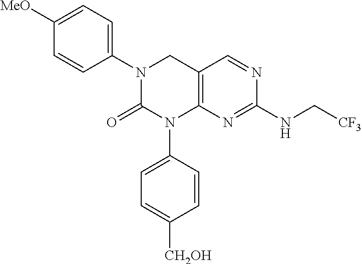 [SMILES: COc1ccc(N2Cc3cnc(NCC(F)(F)F)nc3N(c3ccc(CO)cc3)C2=O)cc1] | 202 |
| 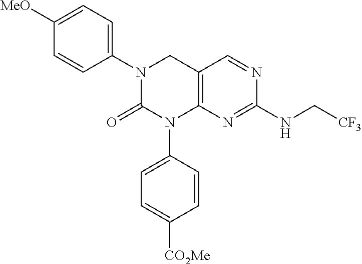 [SMILES: COC(=O)c1ccc(N2C(=O)N(c3ccc(OC)cc3)Cc3cnc(NCC(F)(F)F)nc32)cc1] | 203 |
| 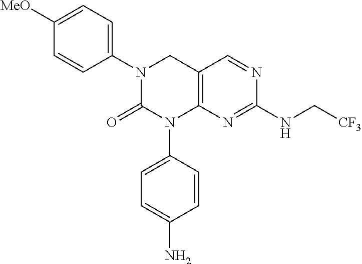 [SMILES: COc1ccc(N2Cc3cnc(NCC(F)(F)F)nc3N(c3ccc(N)cc3)C2=O)cc1] | 204 |
| 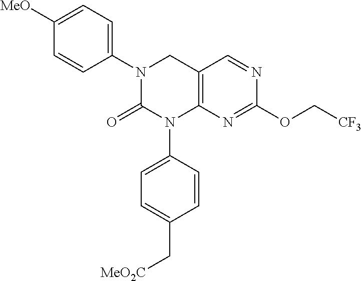 [SMILES: COC(=O)Cc1ccc(N2C(=O)N(c3ccc(OC)cc3)Cc3cnc(OCC(F)(F)F)nc32)cc1] | 205 |
| 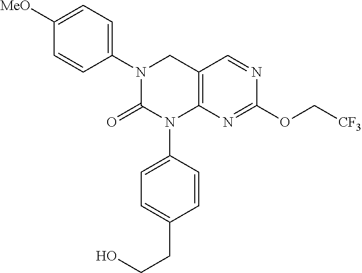 [SMILES: COc1ccc(N2Cc3cnc(OCC(F)(F)F)nc3N(c3ccc(CCO)cc3)C2=O)cc1] | 206 |
| 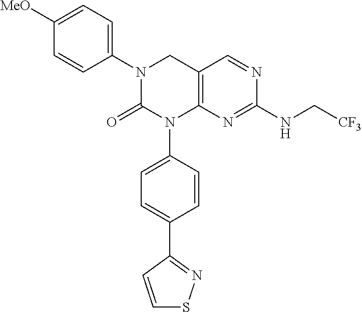 [SMILES: COc1ccc(N2Cc3cnc(NCC(F)(F)F)nc3N(c3ccc(-c4ccsn4)cc3)C2=O)cc1] | 207 |
| 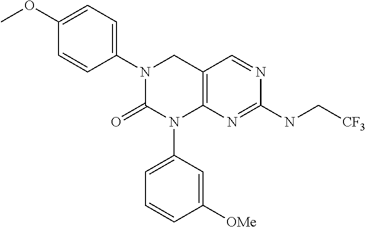 [SMILES: COc1ccc(N2Cc3cnc(NCC(F)(F)F)nc3N(c3cccc(OC)c3)C2=O)cc1] | 208 |
| 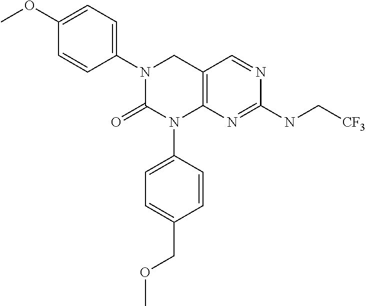 [SMILES: COCc1ccc(N2C(=O)N(c3ccc(OC)cc3)Cc3cnc(NCC(F)(F)F)nc32)cc1] | 209 |
| 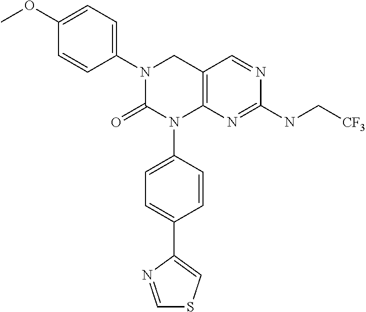 [SMILES: COc1ccc(N2Cc3cnc(NCC(F)(F)F)nc3N(c3ccc(-c4cscn4)cc3)C2=O)cc1] | 210 |
| 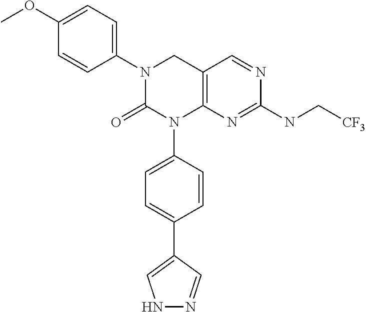 [SMILES: COc1ccc(N2Cc3cnc(NCC(F)(F)F)nc3N(c3ccc(-c4cn[nH]c4)cc3)C2=O)cc1] | 211 |
| 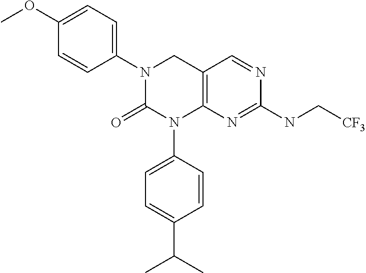 [SMILES: COc1ccc(N2Cc3cnc(NCC(F)(F)F)nc3N(c3ccc(C(C)C)cc3)C2=O)cc1] | 212 |
| 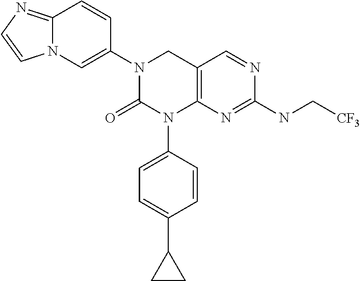 [SMILES: O=C1N(c2ccc3nccn3c2)Cc2cnc(NCC(F)(F)F)nc2N1c1ccc(C2CC2)cc1] | 213 |
| 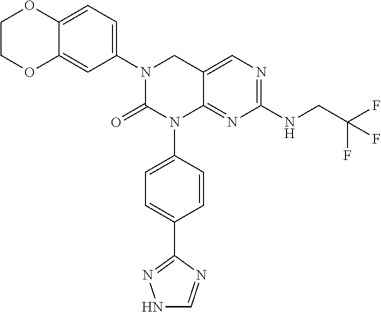 [SMILES: O=C1N(c2ccc3c(c2)OCCO3)Cc2cnc(NCC(F)(F)F)nc2N1c1ccc(-c2nc[nH]n2)cc1] | 214 |
| 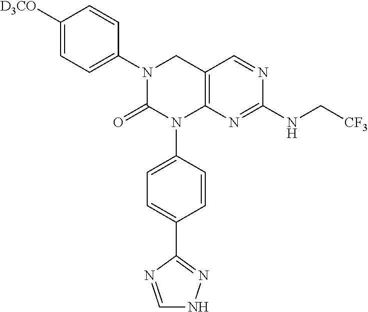 | 215 |
| 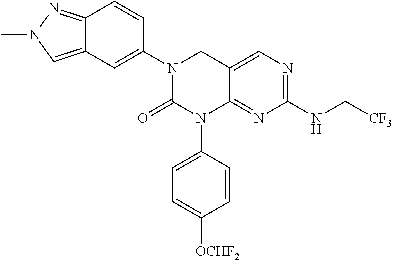 [SMILES: Cn1cc2cc(N3Cc4cnc(NCC(F)(F)F)nc4N(c4ccc(OC(F)F)cc4)C3=O)ccc2n1] | 216 |
| 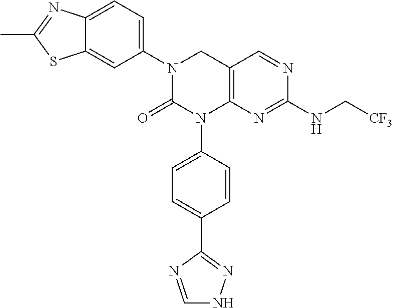 [SMILES: Cc1nc2ccc(N3Cc4cnc(NCC(F)(F)F)nc4N(c4ccc(-c5nc[nH]n5)cc4)C3=O)cc2s1] | 217 |
| 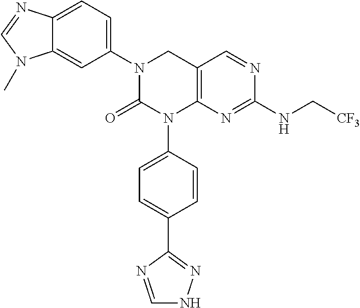 [SMILES: Cn1cnc2ccc(N3Cc4cnc(NCC(F)(F)F)nc4N(c4ccc(-c5nc[nH]n5)cc4)C3=O)cc21] | 218 |
| 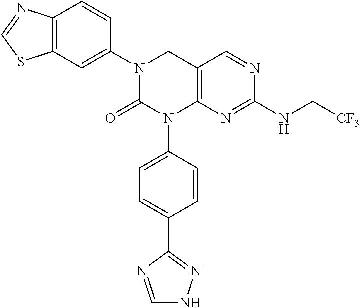 [SMILES: O=C1N(c2ccc3ncsc3c2)Cc2cnc(NCC(F)(F)F)nc2N1c1ccc(-c2nc[nH]n2)cc1] | 219 |
| 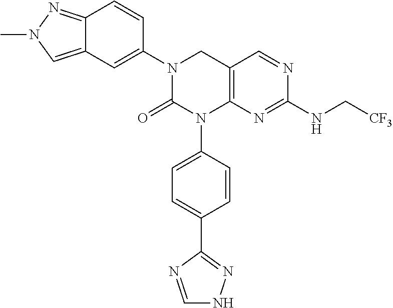 [SMILES: Cn1cc2cc(N3Cc4cnc(NCC(F)(F)F)nc4N(c4ccc(-c5nc[nH]n5)cc4)C3=O)ccc2n1] | 220 |
| 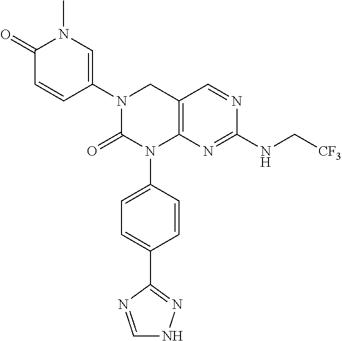 [SMILES: Cn1cc(N2Cc3cnc(NCC(F)(F)F)nc3N(c3ccc(-c4nc[nH]n4)cc3)C2=O)ccc1=O] | 221 |
| 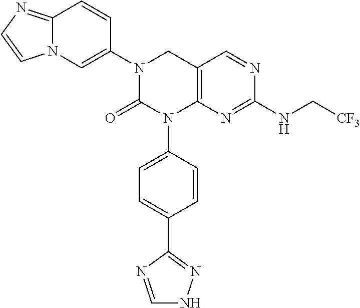 [SMILES: O=C1N(c2ccc3nccn3c2)Cc2cnc(NCC(F)(F)F)nc2N1c1ccc(-c2nc[nH]n2)cc1] | 222 |
| 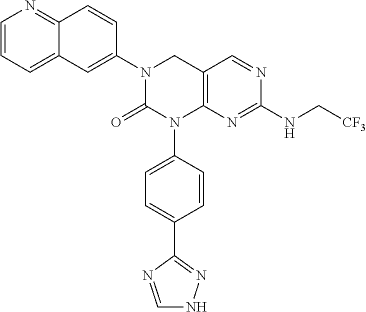 [SMILES: O=C1N(c2ccc3ncccc3c2)Cc2cnc(NCC(F)(F)F)nc2N1c1ccc(-c2nc[nH]n2)cc1] | 223 |
| 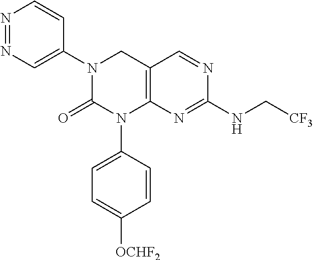 [SMILES: O=C1N(c2ccnnc2)Cc2cnc(NCC(F)(F)F)nc2N1c1ccc(OC(F)F)cc1] | 224 |
| 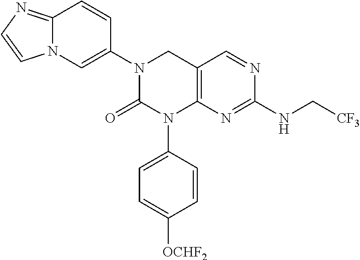 [SMILES: O=C1N(c2ccc3nccn3c2)Cc2cnc(NCC(F)(F)F)nc2N1c1ccc(OC(F)F)cc1] | 225 |
| 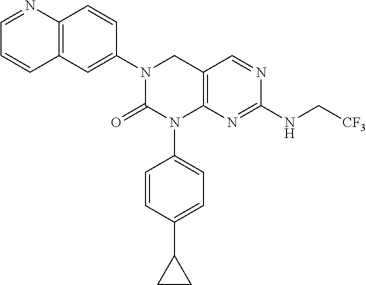 [SMILES: O=C1N(c2ccc3ncccc3c2)Cc2cnc(NCC(F)(F)F)nc2N1c1ccc(C2CC2)cc1] | 226 |
| 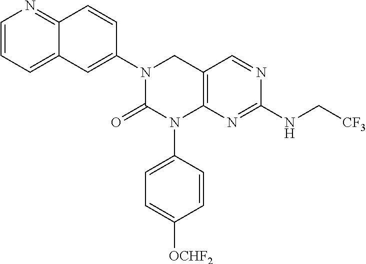 [SMILES: O=C1N(c2ccc3ncccc3c2)Cc2cnc(NCC(F)(F)F)nc2N1c1ccc(OC(F)F)cc1] | 227 |
| 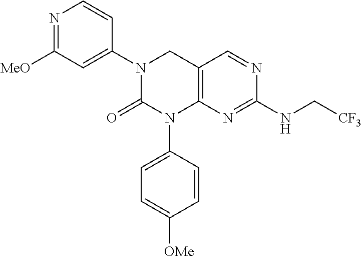 [SMILES: COc1ccc(N2C(=O)N(c3ccnc(OC)c3)Cc3cnc(NCC(F)(F)F)nc32)cc1] | 228 |
| 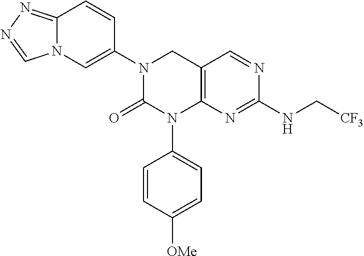 [SMILES: COc1ccc(N2C(=O)N(c3ccc4nncn4c3)Cc3cnc(NCC(F)(F)F)nc32)cc1] | 229 |
| 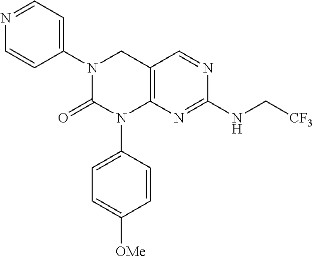 [SMILES: COc1ccc(N2C(=O)N(c3ccncc3)Cc3cnc(NCC(F)(F)F)nc32)cc1] | 230 |
| 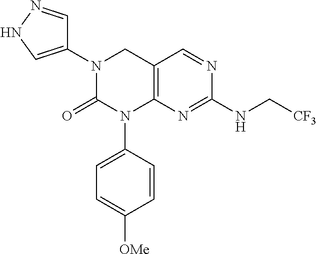 [SMILES: COc1ccc(N2C(=O)N(c3cn[nH]c3)Cc3cnc(NCC(F)(F)F)nc32)cc1] | 231 |
| 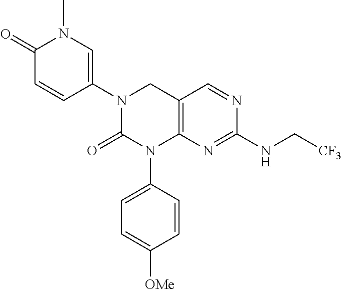 [SMILES: COc1ccc(N2C(=O)N(c3ccc(=O)n(C)c3)Cc3cnc(NCC(F)(F)F)nc32)cc1] | 232 |
| 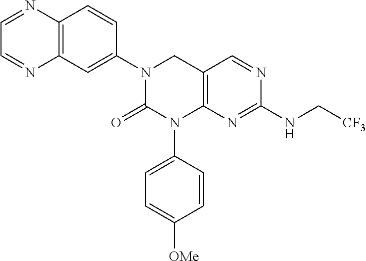 [SMILES: COc1ccc(N2C(=O)N(c3ccc4nccnc4c3)Cc3cnc(NCC(F)(F)F)nc32)cc1] | 233 |
| 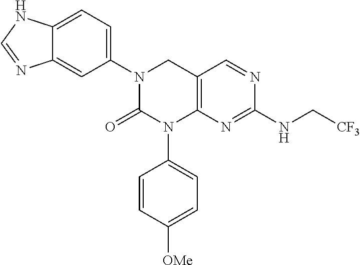 [SMILES: COc1ccc(N2C(=O)N(c3ccc4[nH]cnc4c3)Cc3cnc(NCC(F)(F)F)nc32)cc1] | 234 |
| 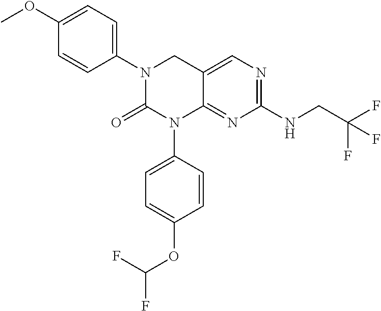 [SMILES: COc1ccc(N2Cc3cnc(NCC(F)(F)F)nc3N(c3ccc(OC(F)F)cc3)C2=O)cc1] | 235 |
| 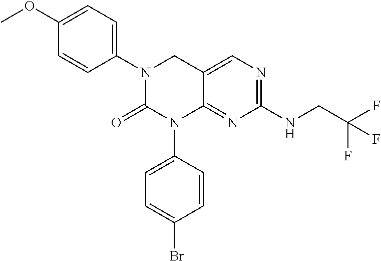 [SMILES: COc1ccc(N2Cc3cnc(NCC(F)(F)F)nc3N(c3ccc(Br)cc3)C2=O)cc1] | 236 |
| 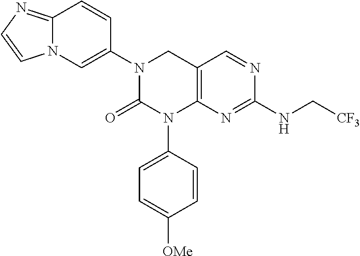 [SMILES: COc1ccc(N2C(=O)N(c3ccc4nccn4c3)Cc3cnc(NCC(F)(F)F)nc32)cc1] | 237 |
| 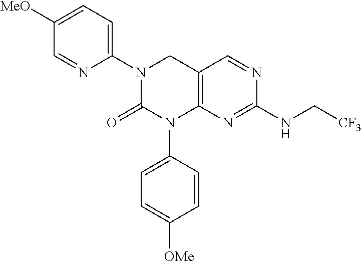 [SMILES: COc1ccc(N2C(=O)N(c3ccc(OC)cn3)Cc3cnc(NCC(F)(F)F)nc32)cc1] | 238 |
| 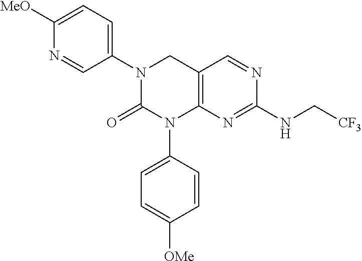 [SMILES: COc1ccc(N2C(=O)N(c3ccc(OC)nc3)Cc3cnc(NCC(F)(F)F)nc32)cc1] | 239 |
| 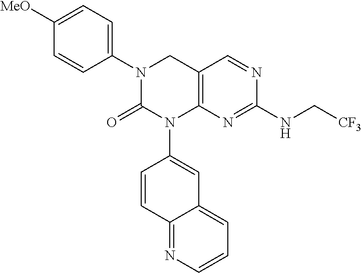 [SMILES: COc1ccc(N2Cc3cnc(NCC(F)(F)F)nc3N(c3ccc4ncccc4c3)C2=O)cc1] | 240 |
| 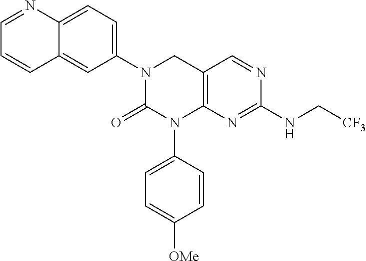 [SMILES: COc1ccc(N2C(=O)N(c3ccc4ncccc4c3)Cc3cnc(NCC(F)(F)F)nc32)cc1] | 241 |
| 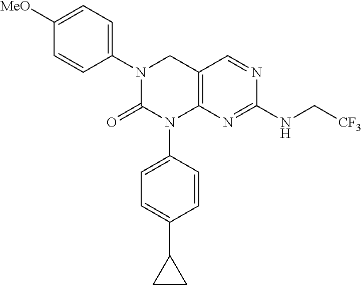 [SMILES: COc1ccc(N2Cc3cnc(NCC(F)(F)F)nc3N(c3ccc(C4CC4)cc3)C2=O)cc1] | 242 |
| 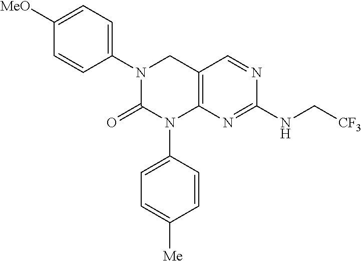 [SMILES: COc1ccc(N2Cc3cnc(NCC(F)(F)F)nc3N(c3ccc(C)cc3)C2=O)cc1] | 243 |
| 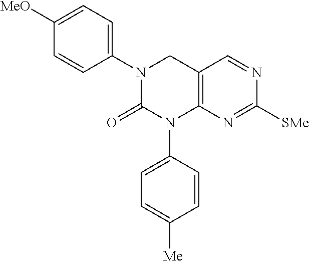 [SMILES: COc1ccc(N2Cc3cnc(SC)nc3N(c3ccc(C)cc3)C2=O)cc1] | 244 |
| 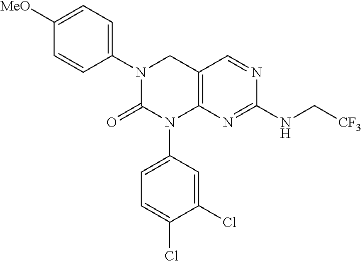 [SMILES: COc1ccc(N2Cc3cnc(NCC(F)(F)F)nc3N(c3ccc(Cl)c(Cl)c3)C2=O)cc1] | 245 |
| 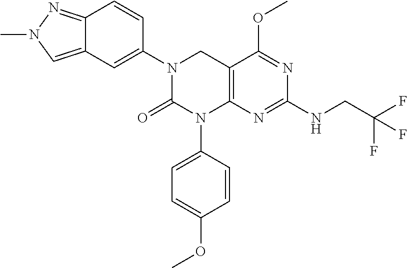 [SMILES: COc1ccc(N2C(=O)N(c3ccc4nn(C)cc4c3)Cc3c(OC)nc(NCC(F)(F)F)nc32)cc1] | 246 |
| 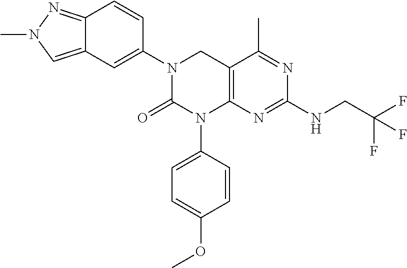 [SMILES: COc1ccc(N2C(=O)N(c3ccc4nn(C)cc4c3)Cc3c(C)nc(NCC(F)(F)F)nc32)cc1] | 247 |
| 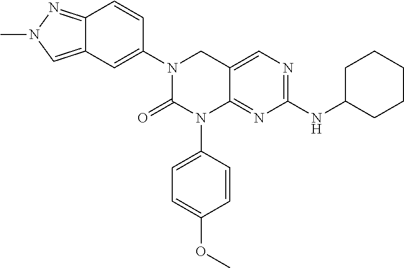 [SMILES: COc1ccc(N2C(=O)N(c3ccc4nn(C)cc4c3)Cc3cnc(NC4CCCCC4)nc32)cc1] | 248 |
| 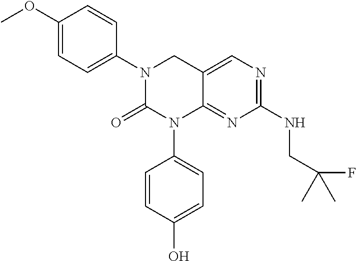 [SMILES: COc1ccc(N2Cc3cnc(NCC(C)(C)F)nc3N(c3ccc(O)cc3)C2=O)cc1] | 249 |
| 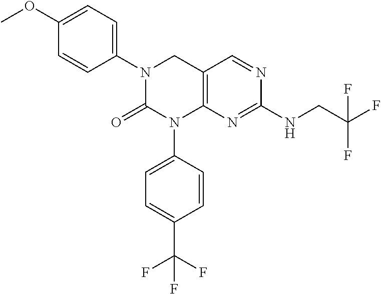 [SMILES: COc1ccc(N2Cc3cnc(NCC(F)(F)F)nc3N(c3ccc(C(F)(F)F)cc3)C2=O)cc1] | 250 |
| 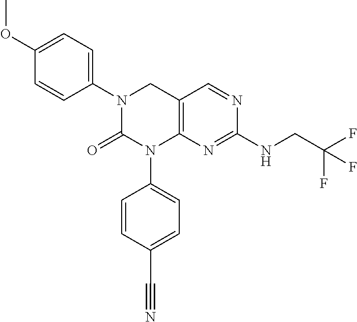 [SMILES: COc1ccc(N2Cc3cnc(NCC(F)(F)F)nc3N(c3ccc(C#N)cc3)C2=O)cc1] | 251 |
| 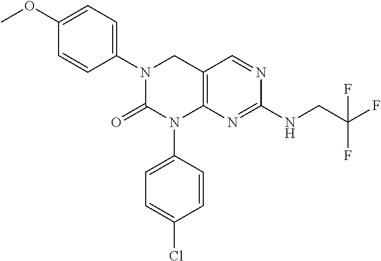 [SMILES: COc1ccc(N2Cc3cnc(NCC(F)(F)F)nc3N(c3ccc(Cl)cc3)C2=O)cc1] | 252 |
| 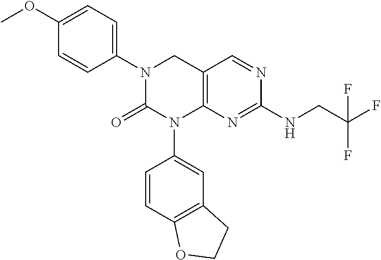 [SMILES: COc1ccc(N2Cc3cnc(NCC(F)(F)F)nc3N(c3ccc4c(c3)CCO4)C2=O)cc1] | 253 |
| 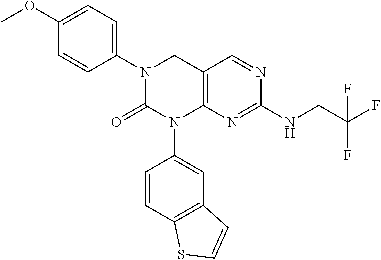 [SMILES: COc1ccc(N2Cc3cnc(NCC(F)(F)F)nc3N(c3ccc4sccc4c3)C2=O)cc1] | 254 |
| 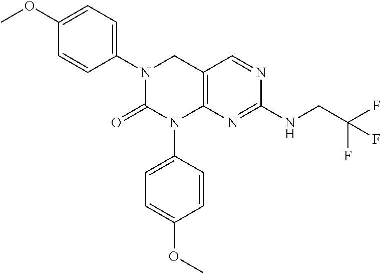 [SMILES: COc1ccc(N2Cc3cnc(NCC(F)(F)F)nc3N(c3ccc(OC)cc3)C2=O)cc1] | 255 |
| 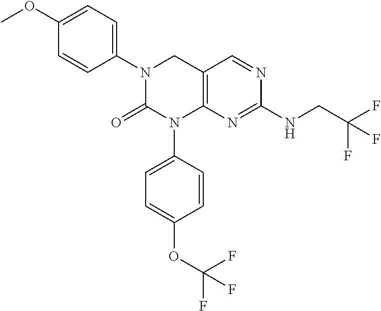 [SMILES: COc1ccc(N2Cc3cnc(NCC(F)(F)F)nc3N(c3ccc(OC(F)(F)F)cc3)C2=O)cc1] | 256 |
| 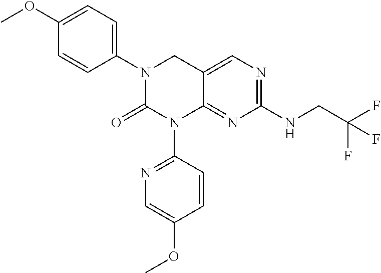 [SMILES: COc1ccc(N2Cc3cnc(NCC(F)(F)F)nc3N(c3ccc(OC)cn3)C2=O)cc1] | 257 |
| 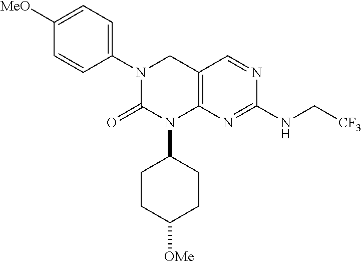 | 258 |
| 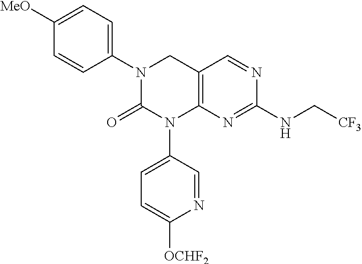 [SMILES: COc1ccc(N2Cc3cnc(NCC(F)(F)F)nc3N(c3ccc(OC(F)F)nc3)C2=O)cc1] | 259 |
| 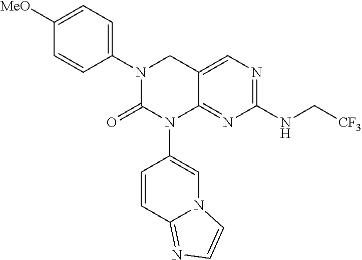 [SMILES: COc1ccc(N2Cc3cnc(NCC(F)(F)F)nc3N(c3ccc4nccn4c3)C2=O)cc1] | 260 |
| 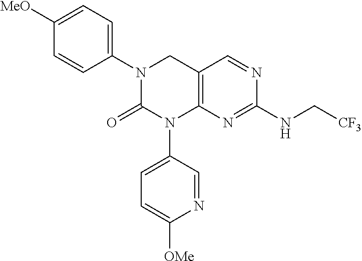 [SMILES: COc1ccc(N2Cc3cnc(NCC(F)(F)F)nc3N(c3ccc(OC)nc3)C2=O)cc1] | 261 |
| 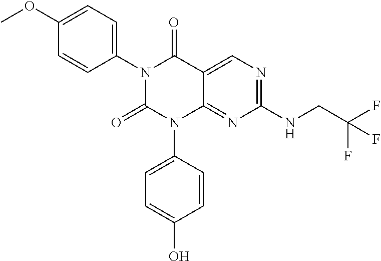 [SMILES: COc1ccc(-n2c(=O)c3cnc(NCC(F)(F)F)nc3n(-c3ccc(O)cc3)c2=O)cc1] | 262 |
| 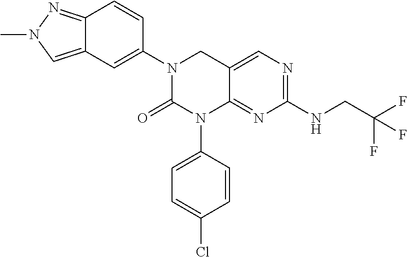 [SMILES: Cn1cc2cc(N3Cc4cnc(NCC(F)(F)F)nc4N(c4ccc(Cl)cc4)C3=O)ccc2n1] | 263 |
| 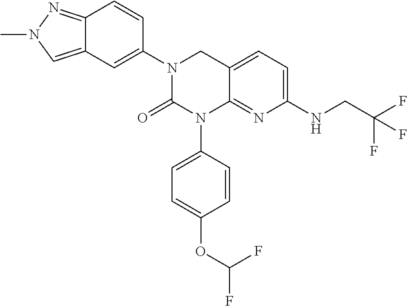 [SMILES: Cn1cc2cc(N3Cc4ccc(NCC(F)(F)F)nc4N(c4ccc(OC(F)F)cc4)C3=O)ccc2n1] | 264 |
| 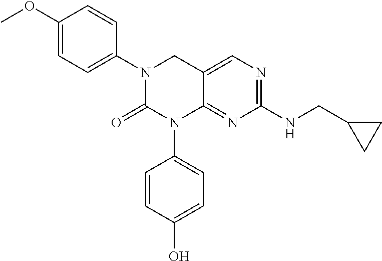 [SMILES: COc1ccc(N2Cc3cnc(NCC4CC4)nc3N(c3ccc(O)cc3)C2=O)cc1] | 265 |
| 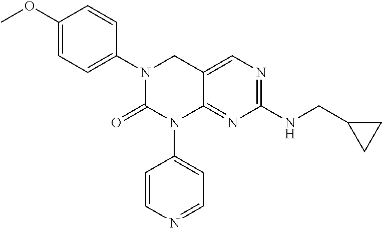 [SMILES: COc1ccc(N2Cc3cnc(NCC4CC4)nc3N(c3ccncc3)C2=O)cc1] | 266 |
| 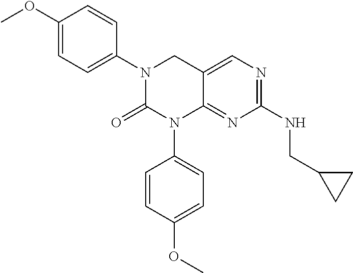 [SMILES: COc1ccc(N2Cc3cnc(NCC4CC4)nc3N(c3ccc(OC)cc3)C2=O)cc1] | 267 |
| 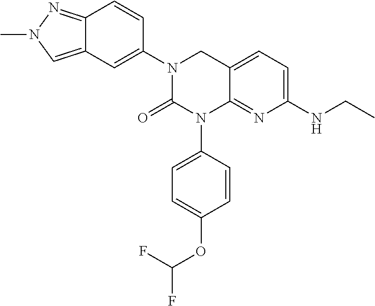 [SMILES: CCNc1ccc2c(n1)N(c1ccc(OC(F)F)cc1)C(=O)N(c1ccc3nn(C)cc3c1)C2] | 269 |
| 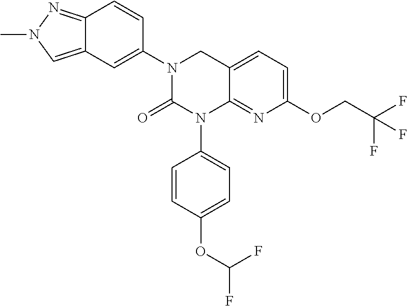 [SMILES: Cn1cc2cc(N3Cc4ccc(OCC(F)(F)F)nc4N(c4ccc(OC(F)F)cc4)C3=O)ccc2n1] | 270 |
| 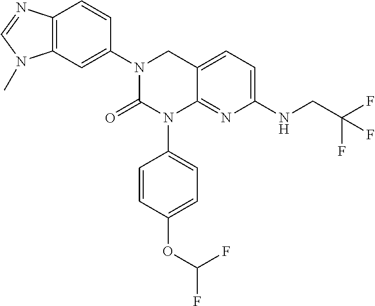 [SMILES: Cn1cnc2ccc(N3Cc4ccc(NCC(F)(F)F)nc4N(c4ccc(OC(F)F)cc4)C3=O)cc21] | 271 |
| 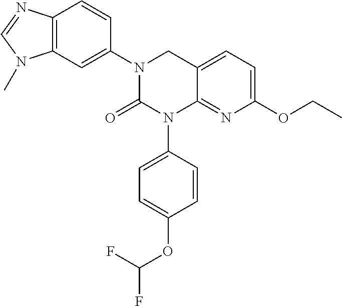 [SMILES: CCOc1ccc2c(n1)N(c1ccc(OC(F)F)cc1)C(=O)N(c1ccc3ncn(C)c3c1)C2] | 272 |
| 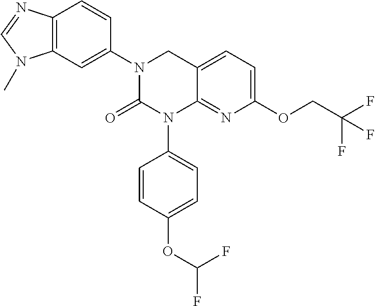 [SMILES: Cn1cnc2ccc(N3Cc4ccc(OCC(F)(F)F)nc4N(c4ccc(OC(F)F)cc4)C3=O)cc21] | 273 |
| 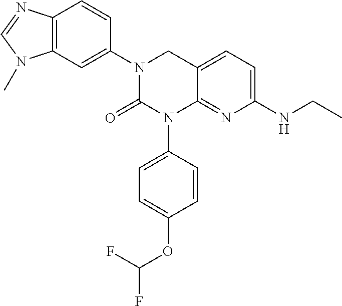 [SMILES: CCNc1ccc2c(n1)N(c1ccc(OC(F)F)cc1)C(=O)N(c1ccc3ncn(C)c3c1)C2] | 274 |
| 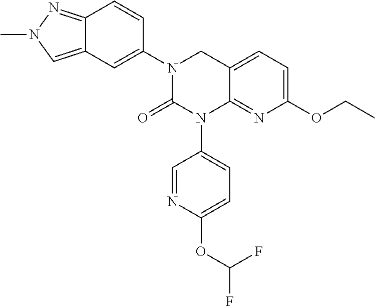 [SMILES: CCOc1ccc2c(n1)N(c1ccc(OC(F)F)nc1)C(=O)N(c1ccc3nn(C)cc3c1)C2] | 275 |
| 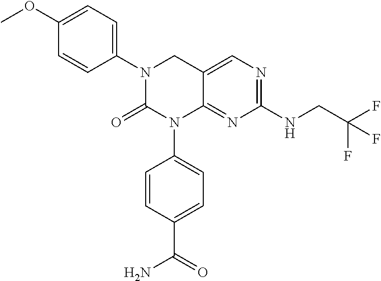 [SMILES: COc1ccc(N2Cc3cnc(NCC(F)(F)F)nc3N(c3ccc(C(N)=O)cc3)C2=O)cc1] | 276 |
| 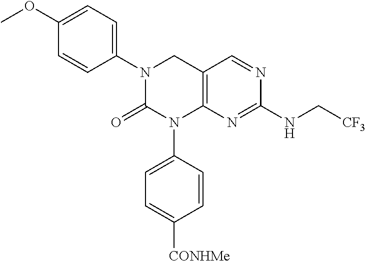 | 277 |
| 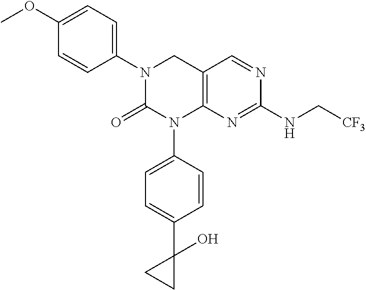 [SMILES: COc1ccc(N2Cc3cnc(NCC(F)(F)F)nc3N(c3ccc(C4(O)CC4)cc3)C2=O)cc1] | 278 |
| 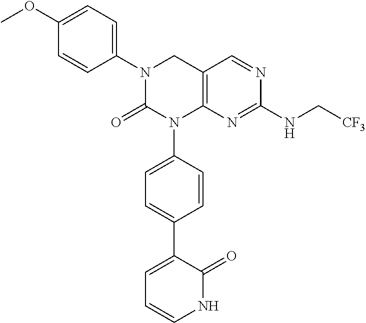 [SMILES: COc1ccc(N2Cc3cnc(NCC(F)(F)F)nc3N(c3ccc(-c4ccc[nH]c4=O)cc3)C2=O)cc1] | 279 |
| 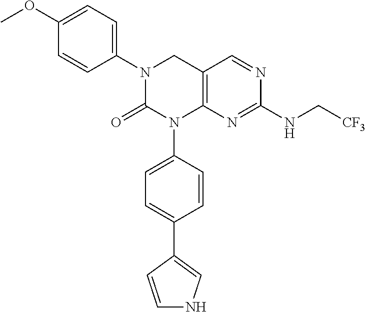 [SMILES: COc1ccc(N2Cc3cnc(NCC(F)(F)F)nc3N(c3ccc(-c4cc[nH]c4)cc3)C2=O)cc1] | 280 |
| 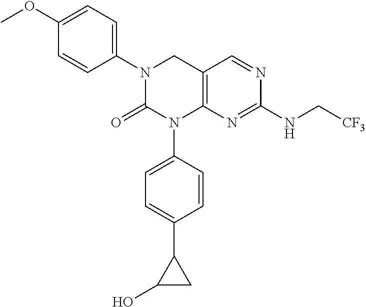 [SMILES: COc1ccc(N2Cc3cnc(NCC(F)(F)F)nc3N(c3ccc(C4CC4O)cc3)C2=O)cc1] | 281 |
| 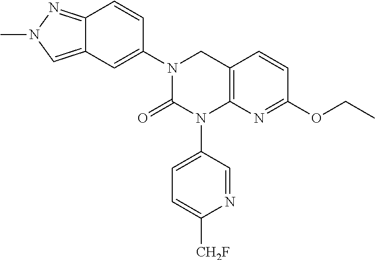 | 282 |
| 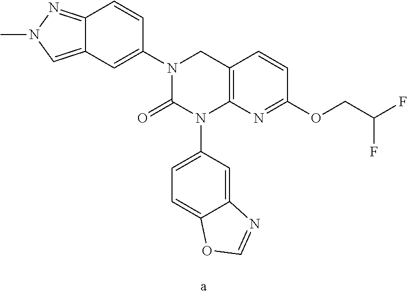 [SMILES: Cn1cc2cc(N3Cc4ccc(OCC(F)F)nc4N(c4ccc5ocnc5c4)C3=O)ccc2n1] | 283 |
| 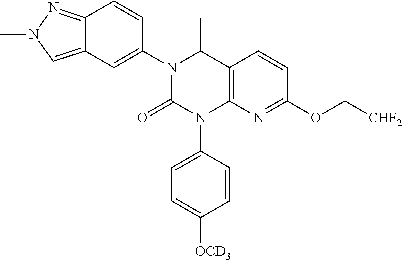 | 286 |
| 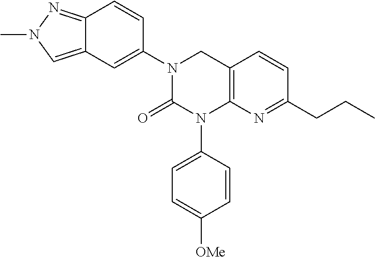 [SMILES: CCCc1ccc2c(n1)N(c1ccc(OC)cc1)C(=O)N(c1ccc3nn(C)cc3c1)C2] | 294 |
| 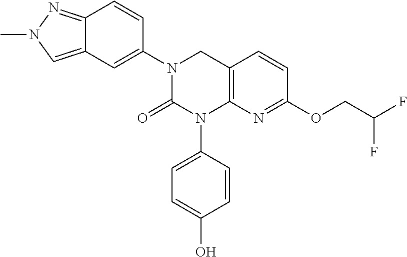 [SMILES: Cn1cc2cc(N3Cc4ccc(OCC(F)F)nc4N(c4ccc(O)cc4)C3=O)ccc2n1] | 295 |

TABLE 2
Examples of Formula II Compounds

| | |
|---|---|
| 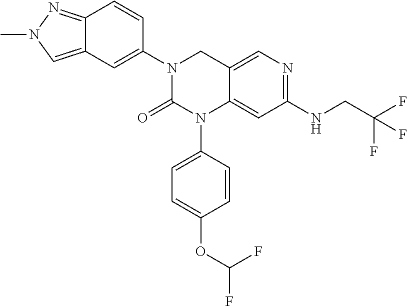 [SMILES: Cn1cc2cc(N3Cc4cnc(NCC(F)(F)F)cc4N(c4ccc(OC(F)F)cc4)C3=O)ccc2n1] | 268 |
| 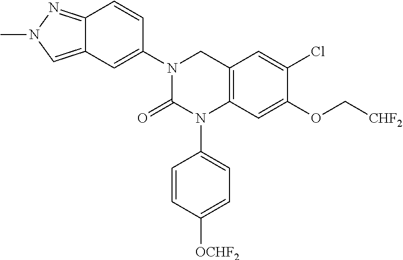 [SMILES: Cn1cc2cc(N3Cc4cc(Cl)c(OCC(F)F)cc4N(c4ccc(OC(F)F)cc4)C3=O)ccc2n1] | 284 |
| 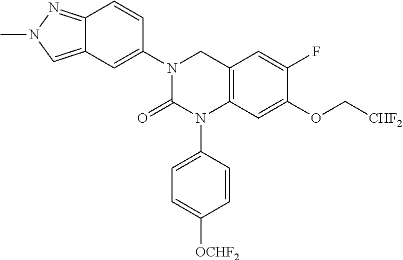 [SMILES: Cn1cc2cc(N3Cc4cc(F)c(OCC(F)F)cc4N(c4ccc(OC(F)F)cc4)C3=O)ccc2n1] | 285 |
| 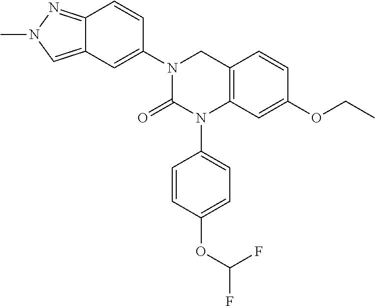 [SMILES: CCOc1ccc2c(c1)N(c1ccc(OC(F)F)cc1)C(=O)N(c1ccc3nn(C)cc3c1)C2] | 287 |
| 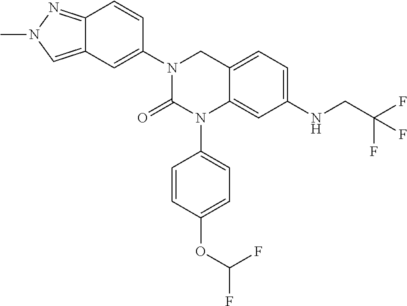 [SMILES: Cn1cc2cc(N3Cc4ccc(NCC(F)(F)F)cc4N(c4ccc(OC(F)F)cc4)C3=O)ccc2n1] | 288 |
| 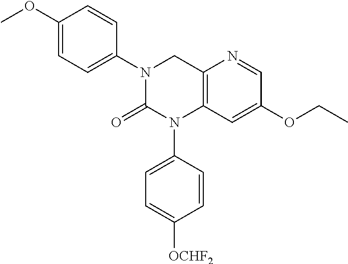 [SMILES: CCOc1cnc2c(c1)N(c1ccc(OC(F)F)cc1)C(=O)N(c1ccc(OC)cc1)C2] | 289 |
| 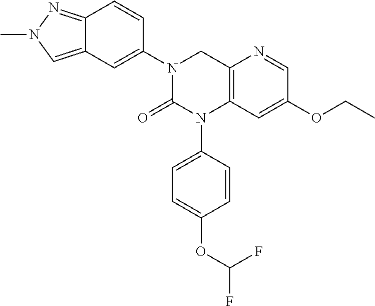 [SMILES: CCOc1cnc2c(c1)N(c1ccc(OC(F)F)cc1)C(=O)N(c1ccc3nn(C)cc3c1)C2] | 290 |
| 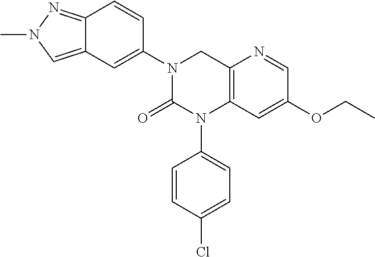 [SMILES: CCOc1cnc2c(c1)N(c1ccc(Cl)cc1)C(=O)N(c1ccc3nn(C)cc3c1)C2] | 291 |
| 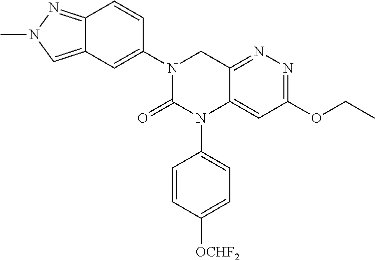 [SMILES: CCOc1cc2c(nn1)CN(c1ccc3nn(C)cc3c1)C(=O)N2c1ccc(OC(F)F)cc1] | 292 |
| 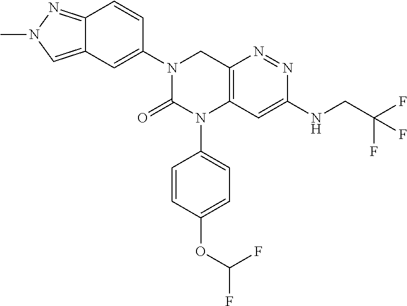 [SMILES: Cn1cc2cc(N3Cc4nnc(NCC(F)(F)F)cc4N(c4ccc(OC(F)F)cc4)C3=O)ccc2n1] | 293 |

TABLE 3
Examples of Formula I and II Compounds

| Ex- | |
|---|---|
| am- | |
| ple | Structure |
| 401 | 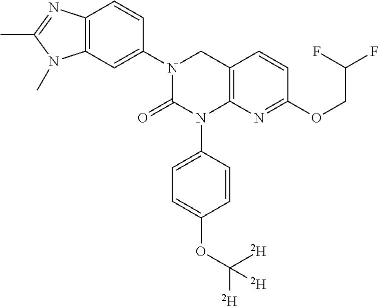 [SMILES: [2H]C([2H])([2H])Oc1ccc(N2C(=O)N(c3ccc4nc(C)n(C)c4c3)Cc3ccc(OCC(F)F)nc32)cc1] |
| 402 | 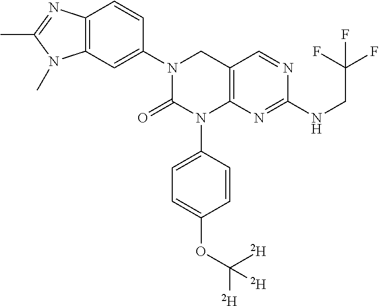 [SMILES: [2H]C([2H])([2H])Oc1ccc(N2C(=O)N(c3ccc4nc(C)n(C)c4c3)Cc3cnc(NCC(F)(F)F)nc32)cc1] |
| 403 | 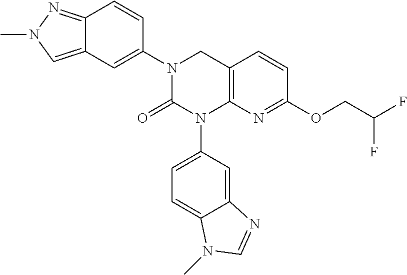 [SMILES: Cn1cc2cc(N3Cc4ccc(OCC(F)F)nc4N(c4ccc5c(c4)ncn5C)C3=O)ccc2n1] |
| 404 | 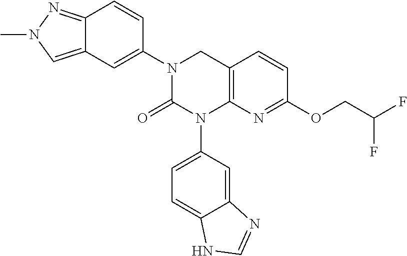 [SMILES: Cn1cc2cc(N3Cc4ccc(OCC(F)F)nc4N(c4ccc5[nH]cnc5c4)C3=O)ccc2n1] |
| 405 | 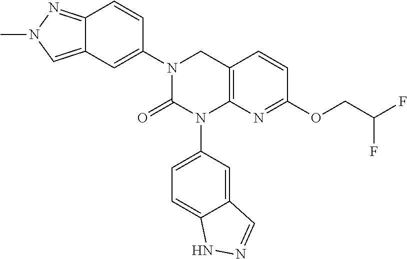 [SMILES: Cn1cc2cc(N3Cc4ccc(OCC(F)F)nc4N(c4ccc5[nH]ncc5c4)C3=O)ccc2n1] |
| 406 | 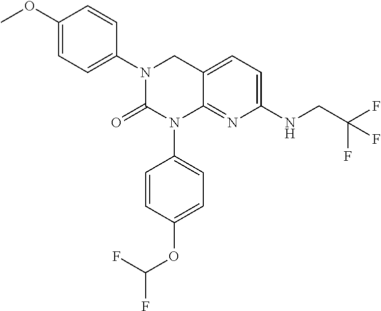 [SMILES: COc1ccc(N2Cc3ccc(NCC(F)(F)F)nc3N(c3ccc(OC(F)F)cc3)C2=O)cc1] |
| 407 | 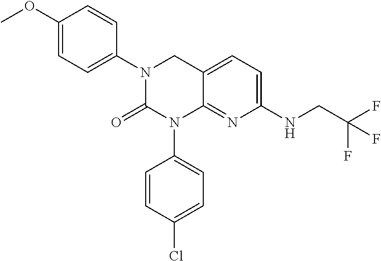 [SMILES: COc1ccc(N2Cc3ccc(NCC(F)(F)F)nc3N(c3ccc(Cl)cc3)C2=O)cc1] |
| 408 | 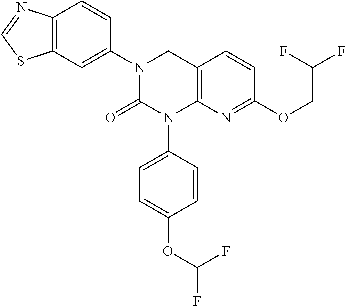 [SMILES: O=C1N(c2ccc3ncsc3c2)Cc2ccc(OCC(F)F)nc2N1c1ccc(OC(F)F)cc1] |
| 409 | 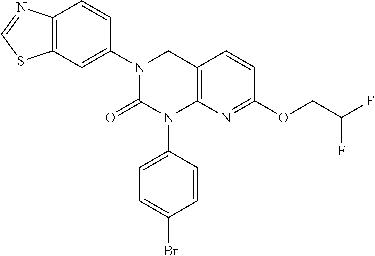 [SMILES: O=C1N(c2ccc3ncsc3c2)Cc2ccc(OCC(F)F)nc2N1c1ccc(Br)cc1] |
| 410 | 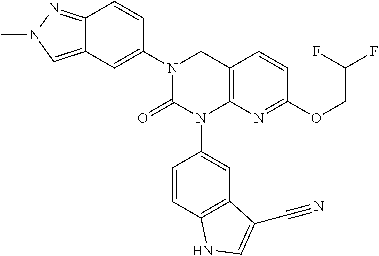 [SMILES: Cn1cc2cc(N3Cc4ccc(OCC(F)F)nc4N(c4ccc5[nH]cc(C#N)c5c4)C3=O)ccc2n1] |
| 411 | 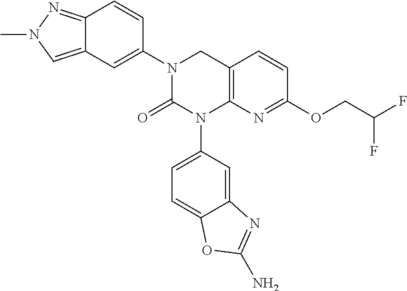 [SMILES: Cn1cc2cc(N3Cc4ccc(OCC(F)F)nc4N(c4ccc5oc(N)nc5c4)C3=O)ccc2n1] |
| 413 | 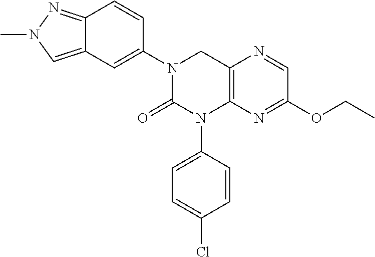 [SMILES: CCOc1cnc2c(n1)N(c1ccc(Cl)cc1)C(=O)N(c1ccc3nn(C)cc3c1)C2] |
| 414 | 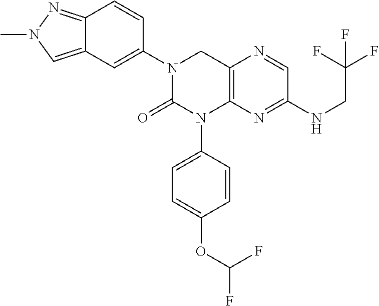 [SMILES: Cn1cc2cc(N3Cc4ncc(NCC(F)(F)F)nc4N(c4ccc(OC(F)F)cc4)C3=O)ccc2n1] |
| 415 | 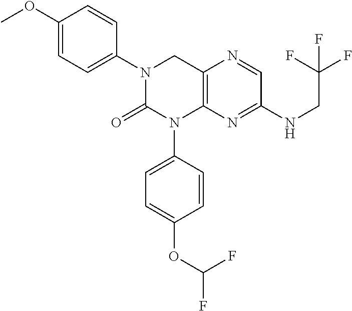 [SMILES: COc1ccc(N2Cc3ncc(NCC(F)(F)F)nc3N(c3ccc(OC(F)F)cc3)C2=O)cc1] |
| 416 | 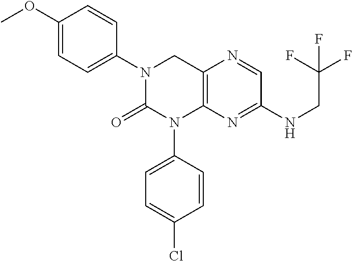 [SMILES: COc1ccc(N2Cc3ncc(NCC(F)(F)F)nc3N(c3ccc(Cl)cc3)C2=O)cc1] |
| 417 | 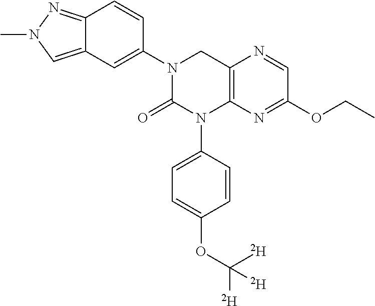 [SMILES: [2H]C([2H])([2H])Oc1ccc(N2C(=O)N(c3ccc4nn(C)cc4c3)Cc3ncc(OCC)nc32)cc1] |
| 418 | 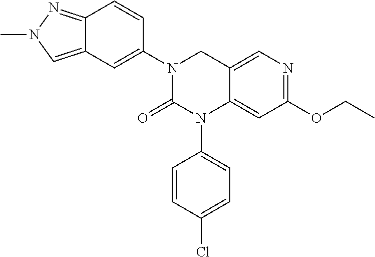 [SMILES: CCOc1cc2c(cn1)CN(c1ccc3nn(C)cc3c1)C(=O)N2c1ccc(Cl)cc1] |
| 419 | 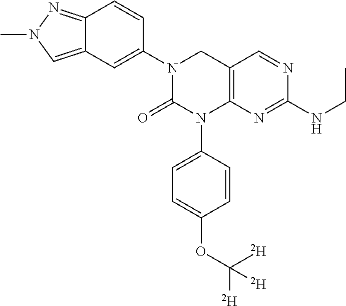 [SMILES: [2H]C([2H])([2H])Oc1ccc(N2C(=O)N(c3ccc4nn(C)cc4c3)Cc3cnc(NCC)nc32)cc1] |
| 420 | 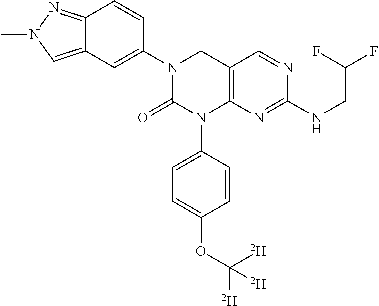 [SMILES: [2H]C([2H])([2H])Oc1ccc(N2C(=O)N(c3ccc4nn(C)cc4c3)Cc3cnc(NCC(F)F)nc32)cc1] |
| 421 | 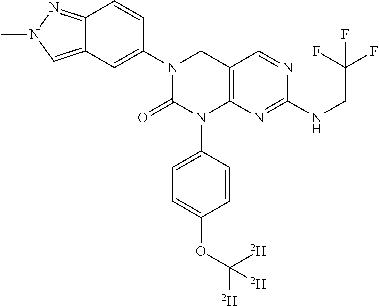 [SMILES: [2H]C([2H])([2H])Oc1ccc(N2C(=O)N(c3ccc4nn(C)cc4c3)Cc3cnc(NCC(F)(F)F)nc32)cc1] |
| 422 | 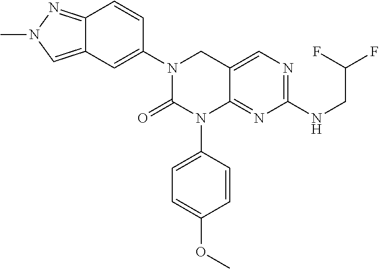 [SMILES: COc1ccc(N2C(=O)N(c3ccc4nn(C)cc4c3)Cc3cnc(NCC(F)F)nc32)cc1] |
| 423 | 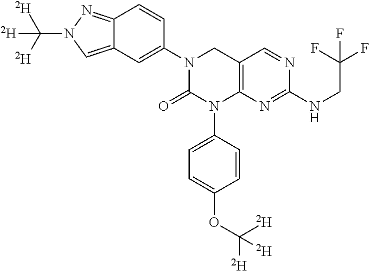 [SMILES: [2H]C([2H])([2H])Oc1ccc(N2C(=O)N(c3ccc4nn(C([2H])([2H])[2H])cc4c3)Cc3cnc(NCC(F)(F)F)nc32)cc1] |
| 424 | 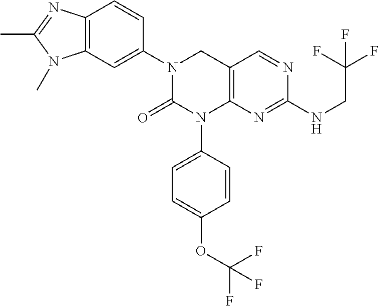 [SMILES: Cc1nc2ccc(N3Cc4cnc(NCC(F)(F)F)nc4N(c4ccc(OC(F)(F)F)cc4)C3=O)cc2n1C] |
| 425 | 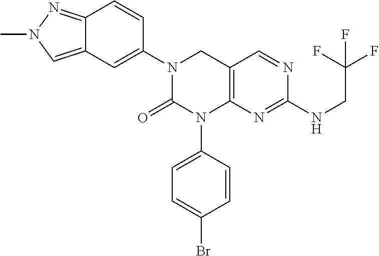 [SMILES: Cn1cc2cc(N3Cc4cnc(NCC(F)(F)F)nc4N(c4ccc(Br)cc4)C3=O)ccc2n1] |
| 427 | 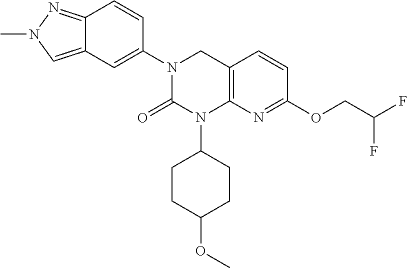 [SMILES: COC1CCC(N2C(=O)N(c3ccc4nn(C)cc4c3)Cc3ccc(OCC(F)F)nc32)CC1] |
| 428 | 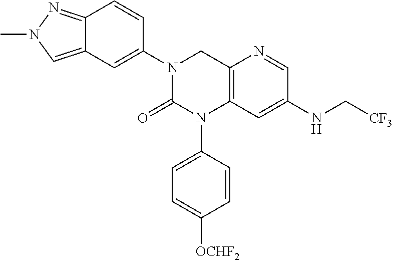 [SMILES: Cn1cc2cc(N3Cc4ncc(NCC(F)(F)F)cc4N(c4ccc(OC(F)F)cc4)C3=O)ccc2n1] |
| 429 | 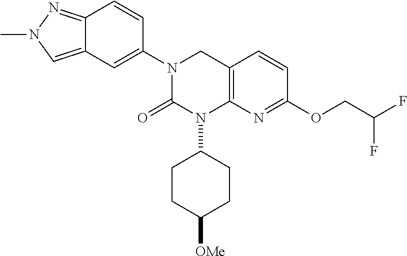 |
| 430 | 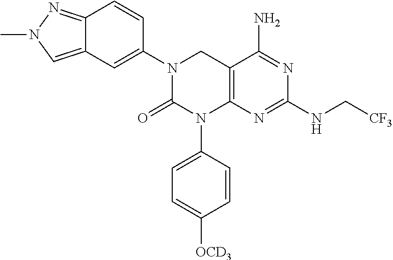 |
| 431 | 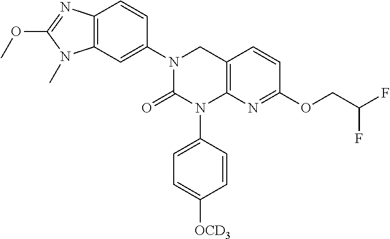 |
| 432 | 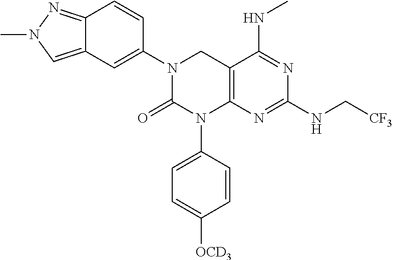 |
| 433 | 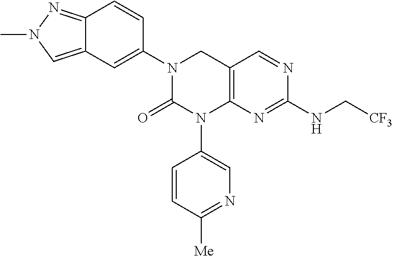 [SMILES: Cc1ccc(N2C(=O)N(c3ccc4nn(C)cc4c3)Cc3cnc(NCC(F)(F)F)nc32)cn1] |
| 434 | 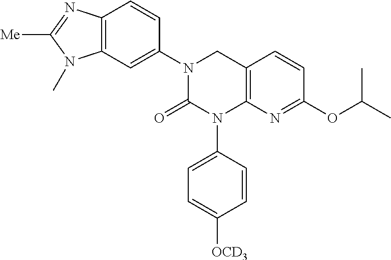 |
| 435 | 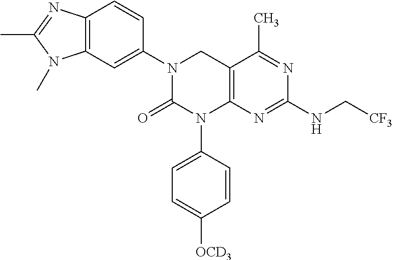 |
| 436 | 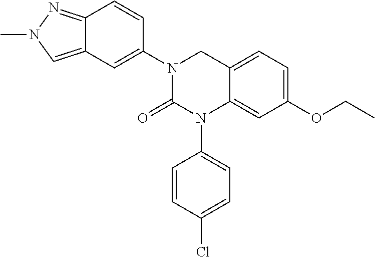 [SMILES: CCOc1ccc2c(c1)N(c1ccc(Cl)cc1)C(=O)N(c1ccc3nn(C)cc3c1)C2] |
| 437 | 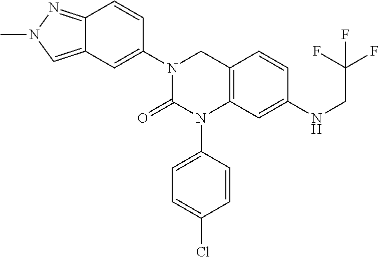 [SMILES: Cn1cc2cc(N3Cc4ccc(NCC(F)(F)F)cc4N(c4ccc(Cl)cc4)C3=O)ccc2n1] |
| 438 | 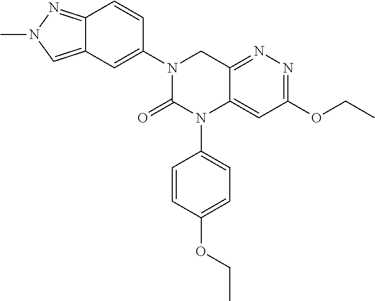 [SMILES: CCOc1ccc(N2C(=O)N(c3ccc4nn(C)cc4c3)Cc3nnc(OCC)cc32)cc1] |

TABLE 4
Example of Formula I Compound

| | |
|---|---|
| 412 | 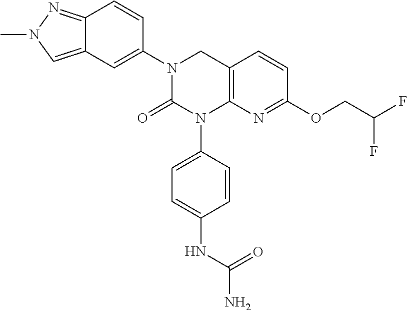 [SMILES: Cn1cc2cc(N3Cc4ccc(OCC(F)F)nc4N(c4ccc(NC(N)=O)cc4)C3=O)ccc2n1] |

### Pharmaceutical Composition

The disclosure also provides a pharmaceutical composition comprising a therapeutically effective amount of one or more compounds according to Formula I, Formula II, or a pharmaceutically acceptable salt, stereoisomer, tautomer, and/or isotopologue thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Tables 1 and 2 or a pharmaceutically acceptable salt, stereoisomer, tautomer, and/or isotopologue thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound (or a pharmaceutically acceptable salt, stereoisomer, tautomer, and/or isotopologue thereof that is administered is governed by such considerations, and is the minimum amount necessary to exert a cytotoxic effect on a cancer, or to inhibit MAT2A activity, or both. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment such dosage forms contain from about 5 mg to about 50 mg of a compound (or a pharmaceutically acceptable salt, stereoisomer, or tautomer thereof) of the present disclosure.

The inventive compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

Inventive compositions suitable for oral use may be prepared according to any method known to the art for the manufacture of pharmaceutical compositions. For instance, liquid formulations of the inventive compounds contain one or more agents selected from the group consisting of sweetening agents, flavoring agents, coloring agents and preserving agents in order to provide pharmaceutically palatable preparations of the MAT2A inhibitor.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation reaction products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I or II may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

The MAT2A enzyme catalyzes the synthesis of S-adenosyl methionine (SAM) from methionine and ATP in cells. Accordingly, in another embodiment of the present disclosure there is provided a method of inhibiting in a cell the synthesis of SAM comprising introducing into the cell an effective amount of a compound of Formula I or II or a pharmaceutically acceptable salt, stereoisomer, tautomer, and/or isotopologue thereof. In other embodiments of the present disclosure there is provided a method of inhibiting in a cell the synthesis of SAM comprising introducing into the cell an effective amount of at least one compound described herein or a pharmaceutically acceptable salt, stereoisomer, tautomer, and/or isotopologue thereof. In some embodiments, the cell is in a subject. In some embodiments, a Formula I or Formula II compound is used to identify other compounds that are inhibitors of MAT2A, for example, in a competition assay for binding to MAT2A or for the inhibition of SAM production. Binding to MAT2A or the inhibition of SAM production by a test compound having a detectable label can be measured with and without the presence of an unlabeled compound of the present disclosure.

The present disclosure also provides a method for treating a cancer in a subject suffering therefrom, comprising administering to the subject an effective amount of a MAT2A inhibitor compound as described herein. In some embodiments, the MAT2A inhibitor is a compound of Formula I or II or a pharmaceutically acceptable salt, stereoisomer, tautomer, and/or isotopologue thereof. In an embodiment, optionally in combination with any other embodiment, the subject is a mammal, such as a human.

In an embodiment, the cancer is an MTAP-deleted cancer. In some embodiments, the cancer as one selected from the group consisting of mesothelioma, neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, head and neck cancer, lymphoma, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma (SCLC), non-small cell lung carcinoma (NSCLC), multiple myeloma (MM), basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

In other embodiments, the cancer is selected from lung cancer, non-small cell lung cancer, bronchioloalviolar cell lung cancer, bone cancer, pancreatic cancer, skin cancer, head and neck cancer, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphoma, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including resistant and/or refractory versions of any of the above cancers, and a combination of one or more of the above cancers.

In some embodiments, the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma and breast cancer.

In other embodiments the lung cancer is non-small cell lung cancer, small cell lung cancer, adenocarcinoma of the lung, and squamous cell carcinoma of the lung.

In other embodiments the breast cancer is triple negative breast cancer (TNBC).

In other embodiments, the brain cancer is a brain tumor selected from the group consisting of glioma, glioblastoma, astrocytoma, meningioma, medulloblastoma, peripheral neuroectodermal tumors, and craniopharyngioma.

In still other embodiments, the cancer is a lymphoma selected from the group consisting of mantle cell lymphoma, Hodgkin lymphoma, non-Hodgkin lymphoma, Burkitt lymphoma, diffuse large B-cell lymphoma (DLBCL), and adult T-cell leukemia/lymphoma (ATLL). As used herein, the expression adult T-cell leukemia/lymphoma refers to a rare and often aggressive T-cell lymphoma that can be found in the blood (leukemia), lymph nodes (lymphoma), skin, or multiple areas of the body.

As described generally above, methylthioadenosine phosphorylase (MTAP) is an enzyme found in all normal tissues that catalyzes the conversion of methylthioadenosine (MTA) into adenine and 5-methylthioribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine. Many human and murine malignant cells lack MTAP activity. MTAP deficiency is not only found in tissue culture cells but the deficiency is also present in primary leukemias, gliomas, melanomas, pancreatic cancers, non-small cell lung cancers (NSCLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesotheliomas. For example, proliferation of cancer cells that are MTAP null, i.e., MTAP-deleted, is inhibited by knocking down MAT2A expression with shRNA which was confirmed using small molecule inhibitors of MAT2A. K. Marjon et al., Cell Reports 15 (2016) 574-587, incorporated herein by reference. An MTAP null or MTAP-deleted cancer is a cancer in which the MTAP gene has been deleted or lost or otherwise deactivated or a cancer in which the MTAP protein has a reduced or impaired function, or a reduced presence.

Accordingly, in an embodiment of the present disclosure there is provided a method for treating a cancer in a subject wherein the cancer is characterized by a reduction or absence of MTAP expression or absence of the MTAP gene or reduced function of MTAP protein as compared to cancers where the MTAP gene and/or protein is present and fully functioning, or as compared to cancers with the wild type MTAP gene. The method comprises administering to the subject a therapeutically effective amount of a compound of Formula I or II or a pharmaceutically acceptable salt, stereoisomer, or tautomer thereof.

In another embodiment, there is provided a method of treating an MTAP deleted cancer in a subject comprising administering to the subject an effective amount of a compound of Formula I, Formula II, or a pharmaceutically acceptable salt, stereoisomer, tautomer, and/or isotopologue thereof. In an embodiment, the MTAP deleted cancer is selected from leukemia, glioma, melanoma, pancreatic cancer, non-small cell lung cancer (NSCLC), bladder cancer, astrocytoma, osteosarcoma, head and neck cancer, myxoid chondrosarcoma, ovarian cancer, endometrial cancer, breast cancer, soft tissue sarcoma, lymphoma, and mesothelioma.

In an embodiment, the MTAP deleted cancer is pancreatic cancer. In another embodiment, the MTAP deleted cancer is selected from bladder cancer, melanoma, brain cancer, lung cancer, pancreatic cancer, breast cancer, liver cancer, esophageal cancer, gastric cancer, colon cancer, head and neck cancer, kidney cancer, colon cancer, diffuse large B cell lymphoma (DLBCL), acute lymphoblastic leukemia (ALL), mantle cell lymphoma (MCL), glioblastoma multiforme (GBM), and non-small cell lung cancer (NSCLC).

Genomic analysis of MTAP null cell lines revealed that cell lines incorporating a KRAS mutation or a p53 mutation were sensitive to MAT2A inhibition. Accordingly, an embodiment of the present disclosure provides a method for treating a cancer in a subject wherein the cancer is characterized by reduction or absence of MTAP expression or absence of the MTAP gene or reduced function of MTAP protein, the method comprising administering to the subject a therapeutically effective amount of a compound of Formula I or II, or a pharmaceutically acceptable salt, stereoisomer, tautomer, and/or isotopologue thereof, wherein said cancer is further characterized by the presence of mutant KRAS or mutant p53. In an embodiment, there is provided a method of treating an MTAP null cancer having a mutant KRAS or mutant p53 in a subject, comprising administering to the subject an effective amount of a compound of Formula I or II or a pharmaceutically acceptable salt, stereoisomer, tautomer, and/or isotopologue thereof. For example, the cancer is MTAP null and KRAS mutant, MTAP null and p53 mutant, or each of MTAP null, KRAS mutant and p53 mutant.

The term “mutant KRAS” or “KRAS mutation” refers to a KRAS protein incorporating an activating mutation that alters its normal function and the gene encoding such a protein. For example, a mutant KRAS protein may incorporate a single amino acid substitution at position 12 or 13. In a particular embodiment, the KRAS mutant incorporates a G12X or G13X substitution, wherein X represents any amino acid change at the indicated position. In a particular embodiment, the substitution is G12V, G12R, G12C or G13D. In another embodiment, the substitution is G13D. By “mutant p53” or “p53 mutation” is meant p53 protein (or gene encoding said protein) incorporating a mutation that inhibits or eliminates its tumor suppressor function. In an embodiment, said p53 mutation is, Y126_splice, K132Q, M133K, R174fs, R175H, R196*, C238S, C242Y, G245S, R248W, R248Q, I255T, D259V, S261_splice, R267P, R273C, R282W, A159V or R280K. In an embodiment, the foregoing cancer is non-small cell lung cancer (NSCLC), pancreatic cancer, head and neck cancer, gastric cancer, breast cancer, colon cancer or ovarian cancer.

In another embodiment, the compounds disclosed herein are useful as ligands for degradation of disease-associated proteins. An example of this approach is PROTACs (PROteolysis TArgeting Chimeras). PROTACs are bifunctional molecules that comprise both a ligand moiety selected from one of the compounds disclosed herein, which is capable of binding the target protein, and a ligase targeting moiety, such as a peptide portion (referred to as the degron) that is recognized and polyubiquitinated by E3 ligase. Thus, the PROTAC non-covalently binds to a target protein, and recruits E3 ligase via the degron, which results in polyubiquination and degradation of the bound target. A number of publications describe the pre-clinical use of PROTACs in a variety of therapeutic areas including oncology. See, e.g., Lu et al. Chemistry & Biology 22 (2015) 755-763.

### ASPECTS

Aspect 1. A compound according to Formula I.

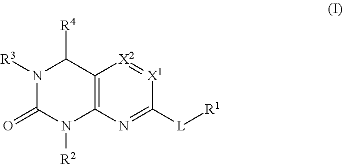

wherein
X¹ is N or CR⁵;
X² is N or CR⁶, wherein X¹ and X² are not simultaneously N;
L is O, S, NR, or a bond;
R is H or C₁-C₆-alkyl;
R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein
any alkyl in R¹ is straight or branched,
R¹ is optionally substituted by 1-6 halo; and
when X¹ is N, X² is CR⁶, L is NR or S, R is H, and R¹ is C₁-C₆-alkyl, then R¹ is substituted by 1-6 halo;
or when L is NR, then R and R¹ can be taken together in combination with L to form a 3- to 6-membered heterocycloalkyl (wherein 1-4 ring members are independently selected from N, O, and S) optionally substituted by one or more R^{A};
R² and R³ are independently selected from the group consisting of C₆-C₁₀-aryl, C₃-C₆-carbocyclyl, 5- to 10-membered heteroaryl (wherein 1-4 heteroaryl members are independently selected from N, O, and S), and 3- to 14-membered heterocycloalkyl (wherein 1-4 heterocycloalkyl members are independently selected from N, O, and S),
wherein R² and R³ are independently and optionally substituted by one or more substituents that are selected from the group consisting of R^{A}, OR^{A}, halo, —N═N— R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN;
R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D};
R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D};
R⁶ is selected from the group consisting of H, C₁-C₆-alkyl (optionally substituted by one or more halo), —O(C₁-C₆-alkyl) (optionally substituted by one or more halo), —OH, halo, —CN, —(C₁-C₆-alkyl)NR^{A}R^{B}, and —NR^{A}R^{B};
R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S);
wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂—C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl), wherein each alkyl, alkenyl, aryl, and heterocycloalkyl is optionally substituted with one or more substituents selected from the group consisting of hydroxy, —OC₁-C₆-alkyl, halo, —NH₂, —(C₁-C₆-alkyl)NH₂, —C(O)OH, CN, and oxo,
R^{C} and R^{D} are each independently selected from H and C₁-C₆-alkyl;
or a pharmaceutically acceptable salt thereof.

Aspect 2. A compound according to Formula II.

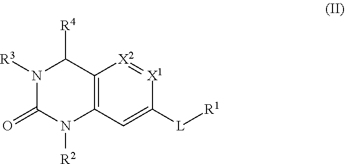

wherein
X¹ is N and X² is CR⁶, or X¹ is CR⁵ and X² is CR⁶, X¹ and X² are both N, or X¹ is CR⁵ and X² is CR⁶;
L is O, S, NR, or a bond;
R is H or C₁-C₆-alkyl;
R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein
any alkyl in R¹ is straight or branched,
R¹ is optionally substituted by 1-6 halo;
or when L is NR, then R and R¹ can be taken together in combination with L to form a 3- to 6-membered heterocycloalkyl (wherein 1-4 ring members are independently selected from N, O, and S) optionally substituted by one or more R^{A};
R² and R³ are independently selected from the group consisting of C₆-C₁₀-aryl, C₃-C₆-carbocyclyl, 5- to 10-membered heteroaryl (wherein 1-4 heteroaryl members are independently selected from N, O, and S), and 3- to 14-membered heterocycloalkyl (wherein 1-4 heterocycloalkyl members are independently selected from N, O, and S),
wherein R² and R³ are independently and optionally substituted by one or more substituents that are selected from the group consisting of R^{A}, OR^{A}, halo, —N═N— R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN;
R⁴ is selected from the group consisting of H, C₁-C₆-alkyl, C₁-C₆-alkoxy, C₂-C₆-alkenyl, C₂-C₆-alkynyl, halo, oxo, —CN, and —NR^{C}R^{D};
R⁵ is selected from the group consisting of H, C₁-C₆-alkyl, C₁-C₆-alkoxy, C₂-C₆-alkenyl, C₂-C₆-alkynyl, halo, —CN, and —NR^{C}R^{D};
R⁶ is selected from the group consisting of H, C₁-C₆-alkyl (optionally substituted by one or more halo), —O(C₁-C₆-alkyl) (optionally substituted by one or more halo), —OH, halo, —CN, —(C₁-C₆-alkyl)NR^{A}R^{B}, and —NR^{A}R^{B};
R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are independently selected from N, O, and S), and 5- to 10-membered heteroaryl (wherein 1-4 heteroaryl members are independently selected from N, O, and S);
wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halo, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl), wherein each alkyl, alkenyl, aryl, and heterocycloalkyl in R^{A} and R^{B} is optionally substituted with one or more substituents selected from the group consisting of hydroxy, —OC₁-C₆-alkyl, halo, —NH₂, —(C₁-C₆-alkyl)NH₂, —C(O)OH, CN, and oxo,
R^{C} and R^{D} are each independently selected from H and C₁-C₆-alkyl;
or a pharmaceutically acceptable salt thereof.

Aspect 3. The compound according to Aspect 1, wherein X¹ is N and X² is CR⁶.

Aspect 4. The compound according to Aspect 1, wherein X¹ is CR⁵ and X² is CR⁶.

Aspect 5. The compound according to Aspect 1, wherein X¹ is CR⁵ and X² is N.

Aspect 6. The compound according to Aspect 2, wherein X¹ is CR⁵ and X² is CR⁶.

Aspect 7. The compound according to Aspect 2, wherein X¹ is N and X² is CR⁶.

Aspect 8. The compound according to Aspect 2, wherein X¹ and X² are both N.

Aspect 9. The compound according to Aspect 2, wherein X¹ is CR⁵ and X² is CR⁶.

Aspect 10. The compound according to any one of Aspects 1-9, wherein each of R⁴ and R⁵ (when present) is independently selected from H and C₁-C₆-alkyl, and R⁶ (when present) is selected from the group consisting of H, C₁-C₆-alkyl optionally substituted by one or more halo, C₁-C₆-alkoxy, —(C₁-C₆-alkyl)NR^{A}R^{B}, and —NR^{A}R^{B} (wherein R^{A} and R^{B} are independently selected from H and C₁-C₆-alkyl).

Aspect 11. The compound according to any one of Aspects 1 to 9, wherein at least one of R⁴, R⁵, and R⁶ (when present) is H.

Aspect 12. The compound according to any one of Aspects 1 to 11, wherein R⁴ is H.

Aspect 13. The compound according to any one of Aspects 1 to 11, wherein R⁵ is H.

Aspect 14. The compound according to any one of Aspects 1 to 11, wherein R⁶ is H.

Aspect 15. The compound according to any one of Aspects 1 to 14, wherein each of R⁴, R⁵, and R⁶ (when present) is H.

Aspect 16. The compound according to any one of Aspects 1 to 15, wherein R² is C₆-C₁₀-aryl or 5- to 10-membered heteroaryl.

Aspect 17. The compound according to Aspect 16, wherein R² is C₆-C₁₀-aryl.

Aspect 18. The compound according to Aspect 17, wherein R² is phenyl.

Aspect 19. The compound according to Aspect 16, wherein R² is 5- to 10-membered heteroaryl, and wherein 1 ring member is N.

Aspect 20. The compound according to Aspect 19, wherein R² is pyridyl.

Aspect 21. The compound according to any one of Aspects 1 to 20, wherein R³ is 3- to 14-membered heterocycloalkyl or 5- to 10-membered heteroaryl.

Aspect 22. The compound according to Aspect 21, wherein R³ is selected from the group consisting of benzothiazolyl, benzoisothiazolyl, benzoxazolyl, pyridinyl, pyridinonyl, pyradazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl.

Aspect 23. The compound according to any one of Aspects 1 to 20, wherein R³ is C₆-C₁₀-aryl.

Aspect 24. The compound according to Aspect 23, wherein R³ is phenyl.

Aspect 25. The compound according to any one of Aspects 1 to 15, wherein R² is phenyl and R³ is 3- to 14-membered heterocycloalkyl or 5- to 10-membered heteroaryl.

Aspect 26. The compound according to any one of Aspects 1 to 25, wherein L is O or NR.

Aspect 27. The compound according to Aspect 26, wherein R¹ is C₁-C₆-alkyl or C₃-C₆-carbocyclyl.

Aspect 28. The compound according to Aspect 26 or 27, wherein R¹ is C₁-C₃-alkyl that is optionally substituted by 1-3 F.

Aspect 29. The compound according to any one of Aspects 1-9, wherein
L is O or NR and R is H;
R¹ is C₁-C₃-alkyl that is optionally substituted by 1-3 F;
R² is 3- to 14-membered heterocycloalkyl or 5- to 10-membered heteroaryl (wherein 1 heterocycloalkyl or heteroaryl member is N) or C₆-C₁₀-aryl;
R³ is 3- to 14-membered heterocycloalkyl, 5- to 10-membered heteroaryl wherein 1 to 3 heterocycloalkyl or heteroaryl members are independently selected from N, O, and S, or C₆-C₁₀-aryl; and
each of R⁴, R¹, and R⁶ (when present) is H.

Aspect 30. The compound according to Aspect 29, wherein L is NR.

Aspect 31. The compound according to Aspect 29 or 30, wherein
R² is optionally substituted phenyl; and
R³ is an optionally substituted 5- to 10-membered heteroaryl wherein 1 to 3 heteroaryl members are independently selected from N, O, and S.

Aspect 32. The compound according to Aspect 29 or 30, wherein
R² is an optionally substituted 5- to 10-membered heteroaryl wherein 1 to 3 heteroaryl members are independently selected from N, O, and S; and
R³ is optionally substituted phenyl.

Aspect 33. The compound according to Aspect 31, wherein R³ is selected from the group consisting of optionally substituted benzothiazolyl, benzoisothiazolyl, benzoxazolyl, pyridinyl, pyridinonyl, pyradazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl.

Aspect 34. The compound according to Aspect 29 or 30, wherein R² and R³ independently are optionally substituted phenyl.

Aspect 35. The compound according to Aspect 1, wherein the compound is selected from the following table:

| | |
|---|---|
| 101 | 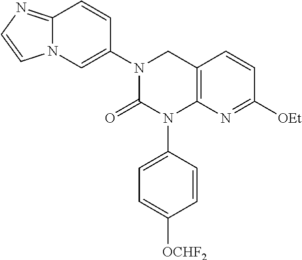 [SMILES: CCOc1ccc2c(n1)N(c1ccc(OC(F)F)cc1)C(=O)N(c1ccc3nccn3c1)C2] |
| 102 | 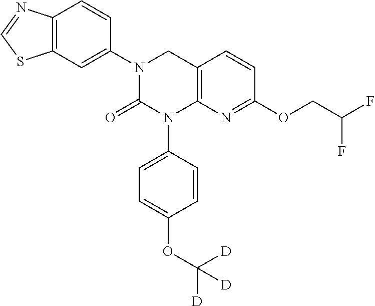 [SMILES: [2H]C([2H])([2H])Oc1ccc(N2C(=O)N(c3ccc4ncsc4c3)Cc3ccc(OCC(F)F)nc32)cc1] |
| 103 | 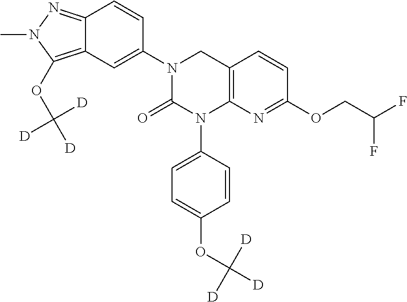 [SMILES: [2H]C([2H])([2H])Oc1ccc(N2C(=O)N(c3ccc4nn(C)c(OC([2H])([2H])[2H])c4c3)Cc3ccc(OCC(F)F)nc32)cc1] |
| 104 | 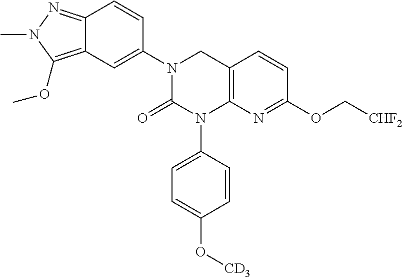 [SMILES: [2H]C([2H])([2H])Oc1ccc(N2C(=O)N(c3ccc4nn(C)c(OC)c4c3)Cc3ccc(OCC(F)F)nc32)cc1] |
| 105 | 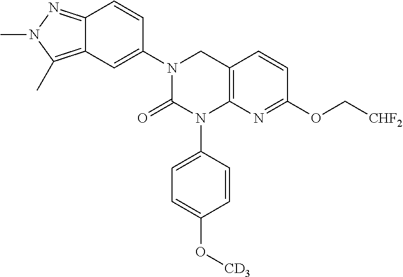 [SMILES: [2H]C([2H])([2H])Oc1ccc(N2C(=O)N(c3ccc4nn(C)c(C)c4c3)Cc3ccc(OCC(F)F)nc32)cc1] |
| 106 | 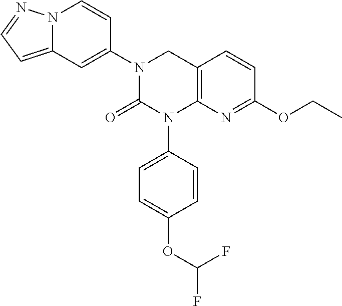 [SMILES: CCOc1ccc2c(n1)N(c1ccc(OC(F)F)cc1)C(=O)N(c1ccn3nccc3c1)C2] |
| 107 | 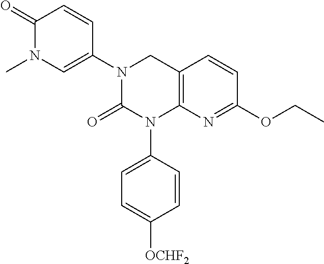 [SMILES: CCOc1ccc2c(n1)N(c1ccc(OC(F)F)cc1)C(=O)N(c1ccc(=O)n(C)c1)C2] |
| 108 | 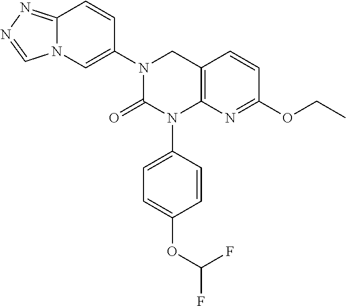 [SMILES: CCOc1ccc2c(n1)N(c1ccc(OC(F)F)cc1)C(=O)N(c1ccc3nncn3c1)C2] |
| 109 | 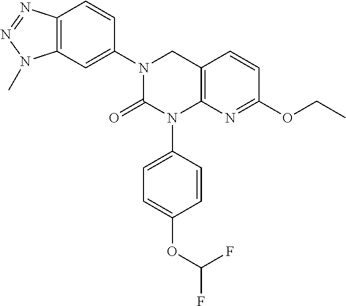 [SMILES: CCOc1ccc2c(n1)N(c1ccc(OC(F)F)cc1)C(=O)N(c1ccc3nnn(C)c3c1)C2] |
| 110 | 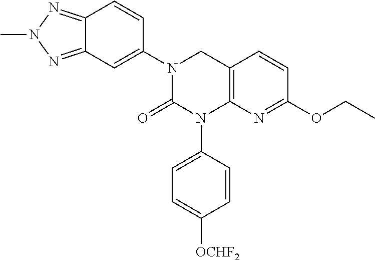 [SMILES: CCOc1ccc2c(n1)N(c1ccc(OC(F)F)cc1)C(=O)N(c1ccc3nn(C)nc3c1)C2] |
| 111 | 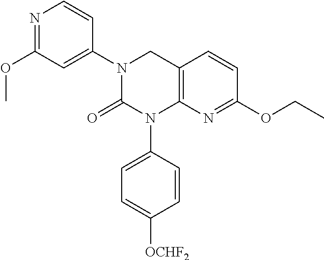 [SMILES: CCOc1ccc2c(n1)N(c1ccc(OC(F)F)cc1)C(=O)N(c1ccnc(OC)c1)C2] |
| 112 | 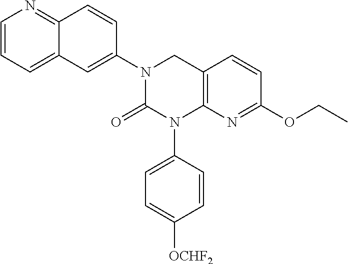 [SMILES: CCOc1ccc2c(n1)N(c1ccc(OC(F)F)cc1)C(=O)N(c1ccc3ncccc3c1)C2] |
| 113 | 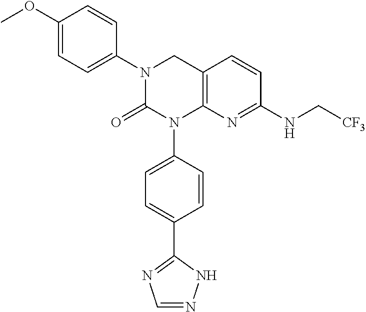 [SMILES: COc1ccc(N2Cc3ccc(NCC(F)(F)F)nc3N(c3ccc(-c4ncn[nH]4)cc3)C2=O)cc1] |
| 114 | 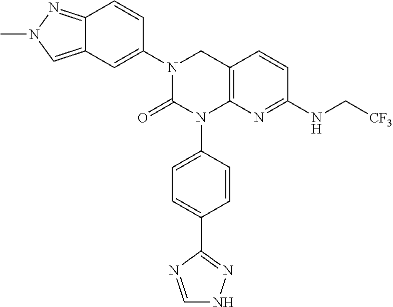 [SMILES: Cn1cc2cc(N3Cc4ccc(NCC(F)(F)F)nc4N(c4ccc(-c5nc[nH]n5)cc4)C3=O)ccc2n1] |
| 115 | 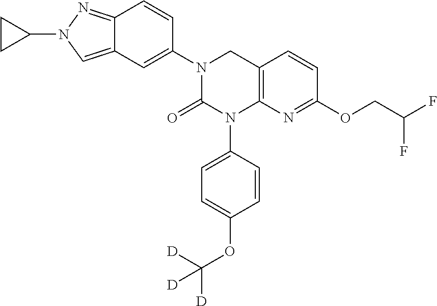 [SMILES: [2H]C([2H])([2H])Oc1ccc(N2C(=O)N(c3ccc4nn(C5CC5)cc4c3)Cc3ccc(OCC(F)F)nc32)cc1] |
| 116 | 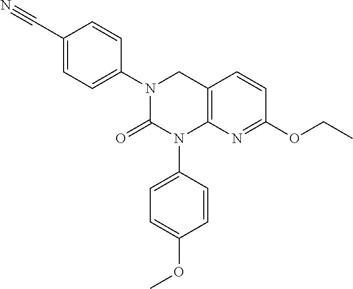 [SMILES: CCOc1ccc2c(n1)N(c1ccc(OC)cc1)C(=O)N(c1ccc(C#N)cc1)C2] |
| 117 | 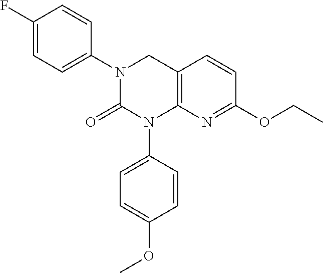 [SMILES: CCOc1ccc2c(n1)N(c1ccc(OC)cc1)C(=O)N(c1ccc(F)cc1)C2] |
| 118 | 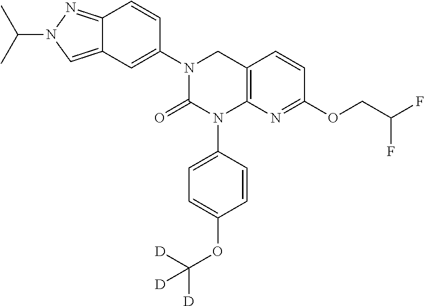 [SMILES: [2H]C([2H])([2H])Oc1ccc(N2C(=O)N(c3ccc4nn(C(C)C)cc4c3)Cc3ccc(OCC(F)F)nc32)cc1] |
| 119 | 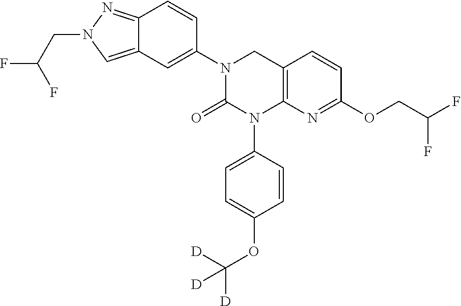 [SMILES: [2H]C([2H])([2H])Oc1ccc(N2C(=O)N(c3ccc4nn(CC(F)F)cc4c3)Cc3ccc(OCC(F)F)nc32)cc1] |
| 120 | 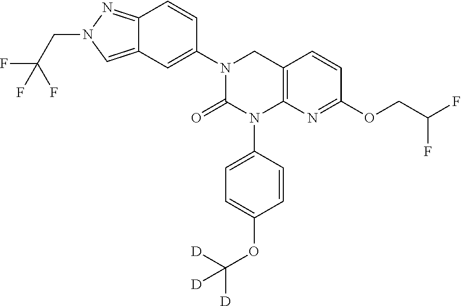 [SMILES: [2H]C([2H])([2H])Oc1ccc(N2C(=O)N(c3ccc4nn(CC(F)(F)F)cc4c3)Cc3ccc(OCC(F)F)nc32)cc1] |
| 121 | 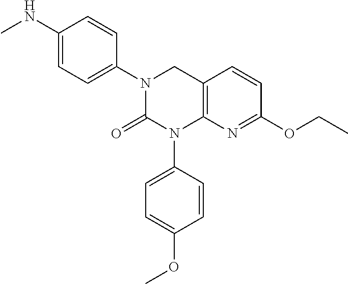 [SMILES: CCOc1ccc2c(n1)N(c1ccc(OC)cc1)C(=O)N(c1ccc(NC)cc1)C2] |
| 122 | 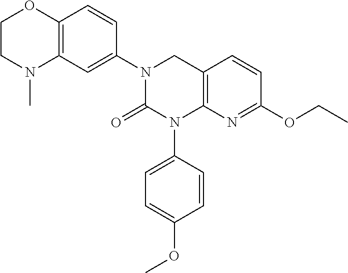 [SMILES: CCOc1ccc2c(n1)N(c1ccc(OC)cc1)C(=O)N(c1ccc3c(c1)N(C)CCO3)C2] |
| 123 | 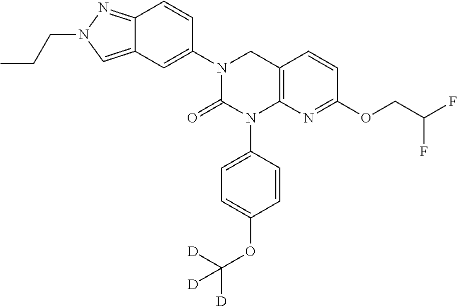 [SMILES: [2H]C([2H])([2H])Oc1ccc(N2C(=O)N(c3ccc4nn(CCC)cc4c3)Cc3ccc(OCC(F)F)nc32)cc1] |
| 124 | 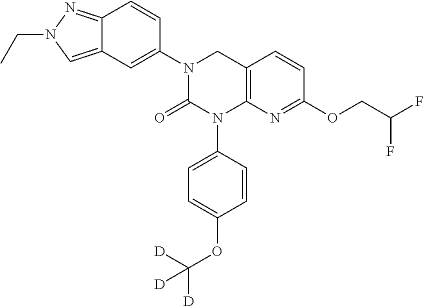 [SMILES: [2H]C([2H])([2H])Oc1ccc(N2C(=O)N(c3ccc4nn(CC)cc4c3)Cc3ccc(OCC(F)F)nc32)cc1] |
| 125 | 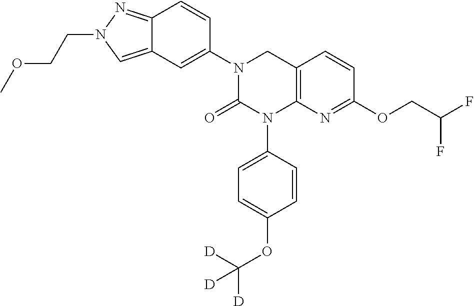 [SMILES: [2H]C([2H])([2H])Oc1ccc(N2C(=O)N(c3ccc4nn(CCOC)cc4c3)Cc3ccc(OCC(F)F)nc32)cc1] |
| 126 | 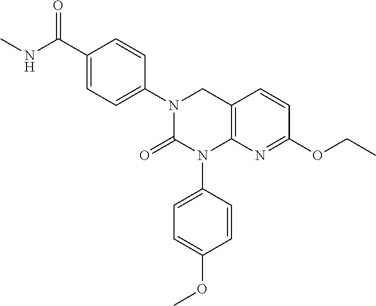 [SMILES: CCOc1ccc2c(n1)N(c1ccc(OC)cc1)C(=O)N(c1ccc(C(=O)NC)cc1)C2] |
| 127 | 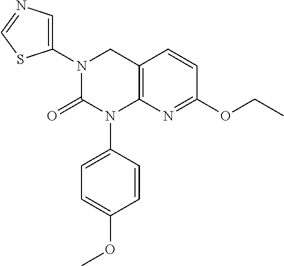 [SMILES: CCOc1ccc2c(n1)N(c1ccc(OC)cc1)C(=O)N(c1cncs1)C2] |
| 128 | 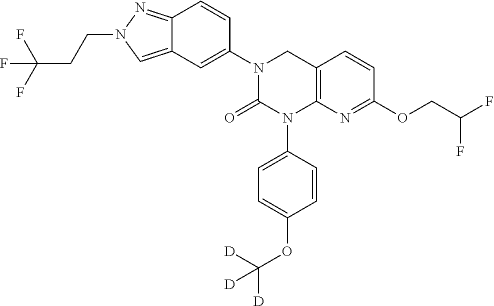 [SMILES: [2H]C([2H])([2H])Oc1ccc(N2C(=O)N(c3ccc4nn(CCC(F)(F)F)cc4c3)Cc3ccc(OCC(F)F)nc32)cc1] |
| 129 | 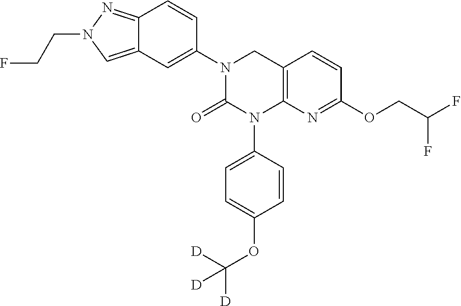 [SMILES: [2H]C([2H])([2H])Oc1ccc(N2C(=O)N(c3ccc4nn(CCF)cc4c3)Cc3ccc(OCC(F)F)nc32)cc1] |
| 130 | 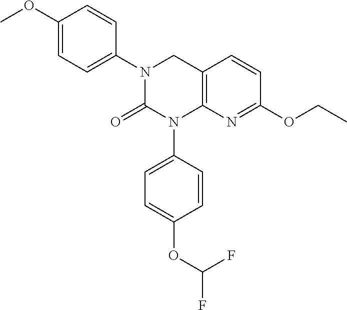 [SMILES: CCOc1ccc2c(n1)N(c1ccc(OC(F)F)cc1)C(=O)N(c1ccc(OC)cc1)C2] |
| 131 | 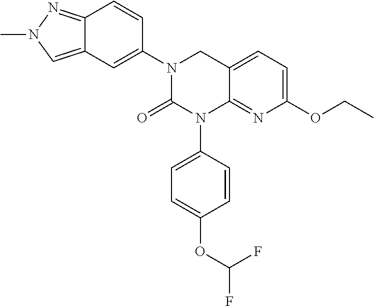 [SMILES: CCOc1ccc2c(n1)N(c1ccc(OC(F)F)cc1)C(=O)N(c1ccc3nn(C)cc3c1)C2] |
| 132 | 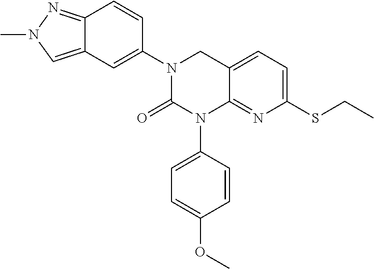 [SMILES: CCSc1ccc2c(n1)N(c1ccc(OC)cc1)C(=O)N(c1ccc3nn(C)cc3c1)C2] |
| 133 | 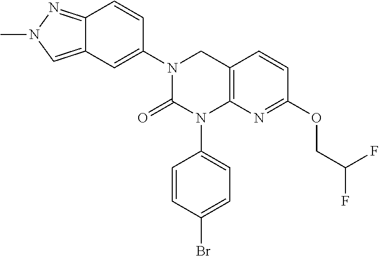 [SMILES: Cn1cc2cc(N3Cc4ccc(OCC(F)F)nc4N(c4ccc(Br)cc4)C3=O)ccc2n1] |
| 135 | 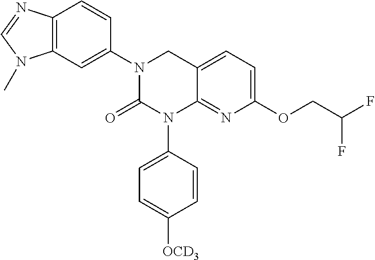 |
| 136 | 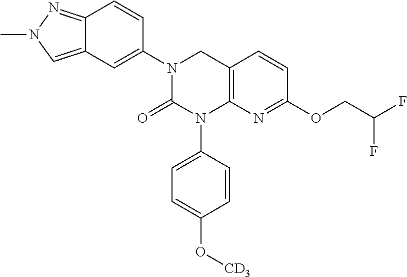 [SMILES: [2H]C([2H])([2H])Oc1ccc(N2C(=O)N(c3ccc4nn(C)cc4c3)Cc3ccc(OCC(F)F)nc32)cc1] |
| 137 | 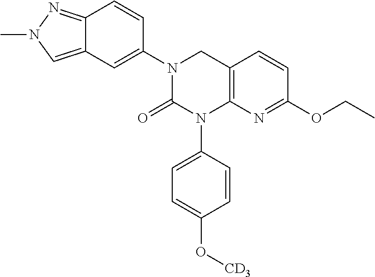 [SMILES: [2H]C([2H])([2H])Oc1ccc(N2C(=O)N(c3ccc4nn(C)cc4c3)Cc3ccc(OCC)nc32)cc1] |
| 138 | 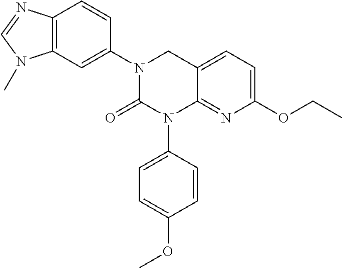 [SMILES: CCOc1ccc2c(n1)N(c1ccc(OC)cc1)C(=O)N(c1ccc3ncn(C)c3c1)C2] |
| 139 | 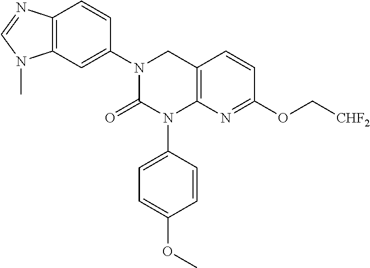 [SMILES: COc1ccc(N2C(=O)N(c3ccc4ncn(C)c4c3)Cc3ccc(OCC(F)F)nc32)cc1] |
| 140 | 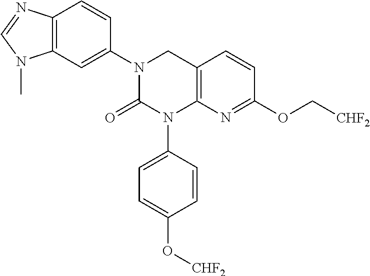 [SMILES: Cn1cnc2ccc(N3Cc4ccc(OCC(F)F)nc4N(c4ccc(OC(F)F)cc4)C3=O)cc21] |
| 141 | 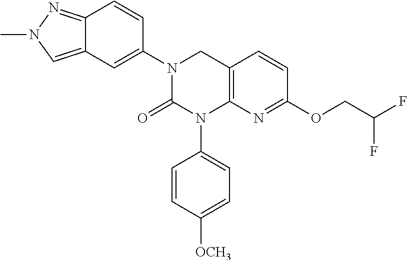 [SMILES: COc1ccc(N2C(=O)N(c3ccc4nn(C)cc4c3)Cc3ccc(OCC(F)F)nc32)cc1] |
| 142 | 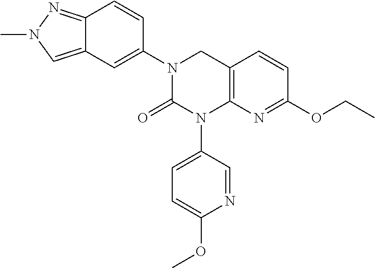 [SMILES: CCOc1ccc2c(n1)N(c1ccc(OC)nc1)C(=O)N(c1ccc3nn(C)cc3c1)C2] |
| 143 | 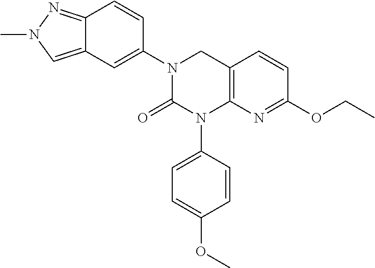 [SMILES: CCOc1ccc2c(n1)N(c1ccc(OC)cc1)C(=O)N(c1ccc3nn(C)cc3c1)C2] |
| 144 | 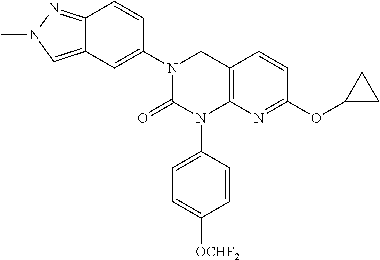 [SMILES: Cn1cc2cc(N3Cc4ccc(OC5CC5)nc4N(c4ccc(OC(F)F)cc4)C3=O)ccc2n1] |
| 145 | 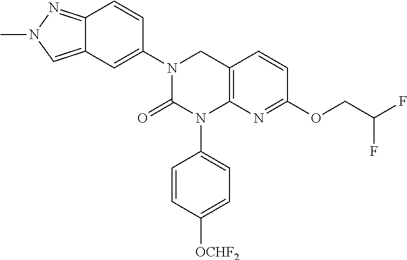 [SMILES: Cn1cc2cc(N3Cc4ccc(OCC(F)F)nc4N(c4ccc(OC(F)F)cc4)C3=O)ccc2n1] |
| 146 | 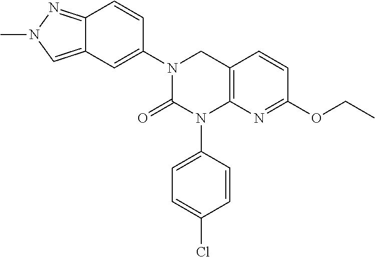 [SMILES: CCOc1ccc2c(n1)N(c1ccc(Cl)cc1)C(=O)N(c1ccc3nn(C)cc3c1)C2] |
| 147 | 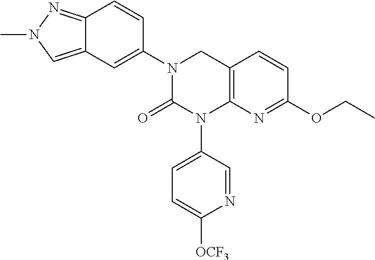 [SMILES: CCOc1ccc2c(n1)N(c1ccc(OC(F)(F)F)nc1)C(=O)N(c1ccc3nn(C)cc3c1)C2] |
| 148 | 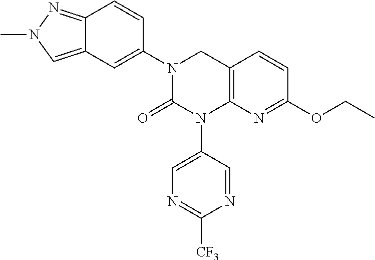 [SMILES: CCOc1ccc2c(n1)N(c1cnc(C(F)(F)F)nc1)C(=O)N(c1ccc3nn(C)cc3c1)C2] |
| 149 | 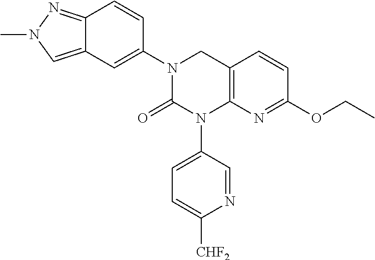 [SMILES: CCOc1ccc2c(n1)N(c1ccc(C(F)F)nc1)C(=O)N(c1ccc3nn(C)cc3c1)C2] |
| 150 | 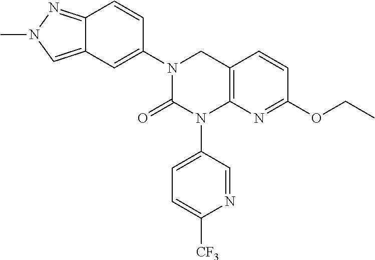 [SMILES: CCOc1ccc2c(n1)N(c1ccc(C(F)(F)F)nc1)C(=O)N(c1ccc3nn(C)cc3c1)C2] |
| 151 | 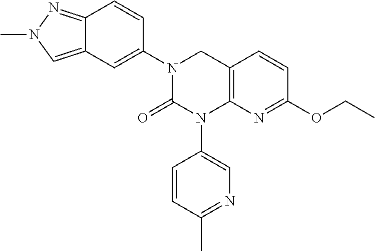 [SMILES: CCOc1ccc2c(n1)N(c1ccc(C)nc1)C(=O)N(c1ccc3nn(C)cc3c1)C2] |
| 152 | 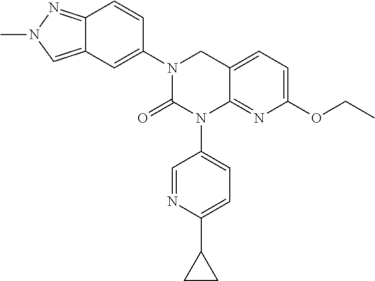 [SMILES: CCOc1ccc2c(n1)N(c1ccc(C3CC3)nc1)C(=O)N(c1ccc3nn(C)cc3c1)C2] |
| 153 | 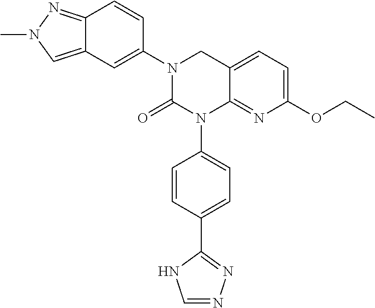 [SMILES: CCOc1ccc2c(n1)N(c1ccc(-c3nnc[nH]3)cc1)C(=O)N(c1ccc3nn(C)cc3c1)C2] |
| 154 | 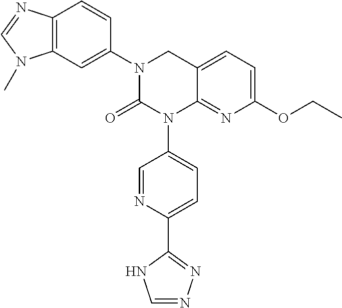 [SMILES: CCOc1ccc2c(n1)N(c1ccc(-c3nnc[nH]3)nc1)C(=O)N(c1ccc3ncn(C)c3c1)C2] |
| 155 | 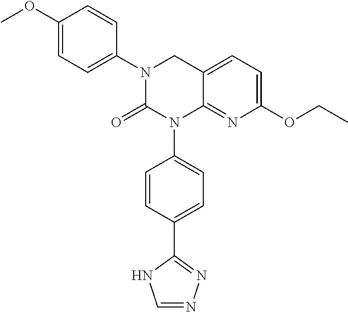 [SMILES: CCOc1ccc2c(n1)N(c1ccc(-c3nnc[nH]3)cc1)C(=O)N(c1ccc(OC)cc1)C2] |
| 157 | 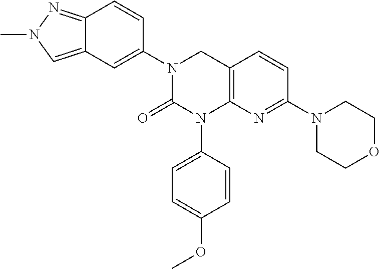 [SMILES: COc1ccc(N2C(=O)N(c3ccc4nn(C)cc4c3)Cc3ccc(N4CCOCC4)nc32)cc1] |
| 158 | 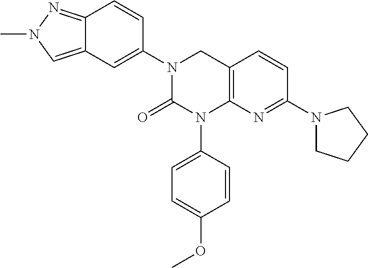 [SMILES: COc1ccc(N2C(=O)N(c3ccc4nn(C)cc4c3)Cc3ccc(N4CCCC4)nc32)cc1] |
| 159 | 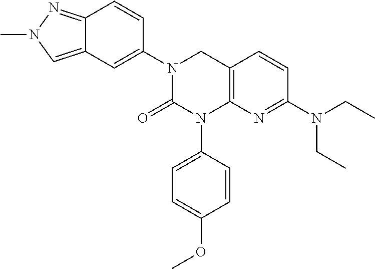 [SMILES: CCN(CC)c1ccc2c(n1)N(c1ccc(OC)cc1)C(=O)N(c1ccc3nn(C)cc3c1)C2] |
| 160 | 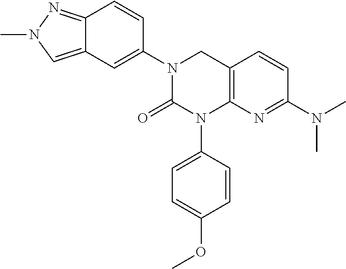 |
| 161 | 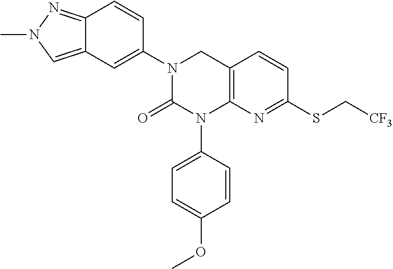 [SMILES: COc1ccc(N2C(=O)N(c3ccc4nn(C)cc4c3)Cc3ccc(SCC(F)(F)F)nc32)cc1] |
| 162 | 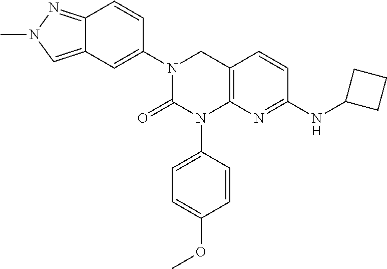 [SMILES: COc1ccc(N2C(=O)N(c3ccc4nn(C)cc4c3)Cc3ccc(NC4CCC4)nc32)cc1] |
| 163 | 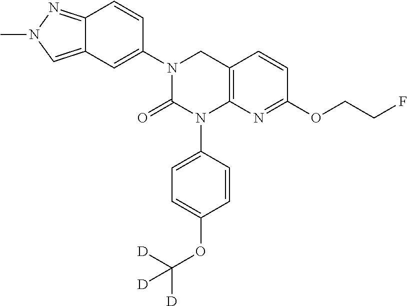 [SMILES: [2H]C([2H])([2H])Oc1ccc(N2C(=O)N(c3ccc4nn(C)cc4c3)Cc3ccc(OCCF)nc32)cc1] |
| 164 | 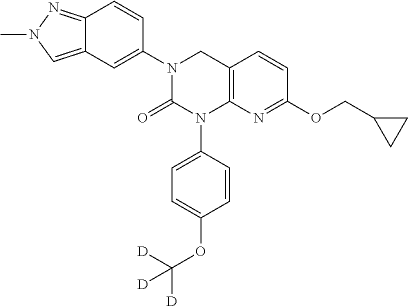 [SMILES: [2H]C([2H])([2H])Oc1ccc(N2C(=O)N(c3ccc4nn(C)cc4c3)Cc3ccc(OCC4CC4)nc32)cc1] |
| 165 | 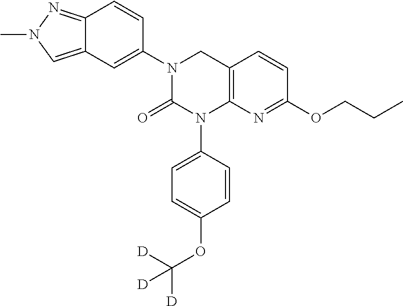 [SMILES: [2H]C([2H])([2H])Oc1ccc(N2C(=O)N(c3ccc4nn(C)cc4c3)Cc3ccc(OCCC)nc32)cc1] |
| 166 | 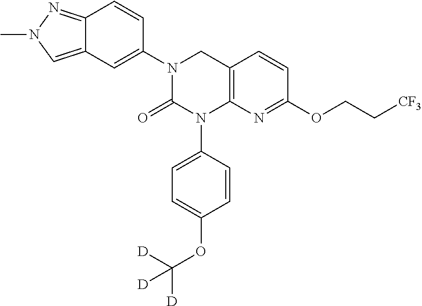 [SMILES: [2H]C([2H])([2H])Oc1ccc(N2C(=O)N(c3ccc4nn(C)cc4c3)Cc3ccc(OCCC(F)(F)F)nc32)cc1] |
| 167 | 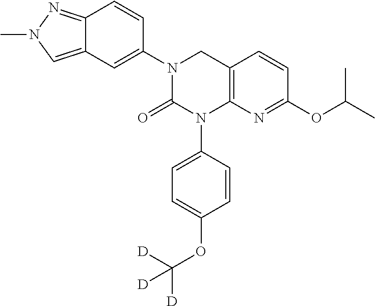 [SMILES: [2H]C([2H])([2H])Oc1ccc(N2C(=O)N(c3ccc4nn(C)cc4c3)Cc3ccc(OC(C)C)nc32)cc1] |
| 168 | 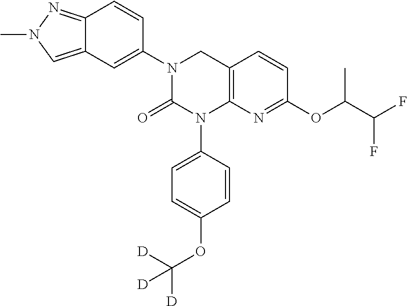 [SMILES: [2H]C([2H])([2H])Oc1ccc(N2C(=O)N(c3ccc4nn(C)cc4c3)Cc3ccc(OC(C)C(F)F)nc32)cc1] |
| 169 | 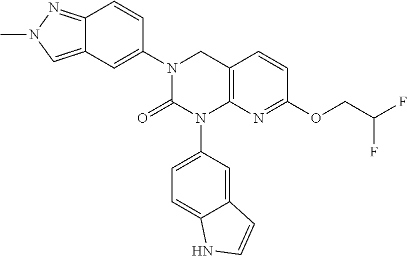 [SMILES: Cn1cc2cc(N3Cc4ccc(OCC(F)F)nc4N(c4ccc5[nH]ccc5c4)C3=O)ccc2n1] |
| 170 | 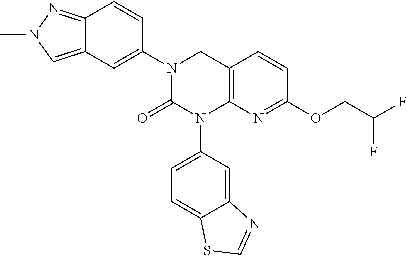 [SMILES: Cn1cc2cc(N3Cc4ccc(OCC(F)F)nc4N(c4ccc5scnc5c4)C3=O)ccc2n1] |
| 171 | 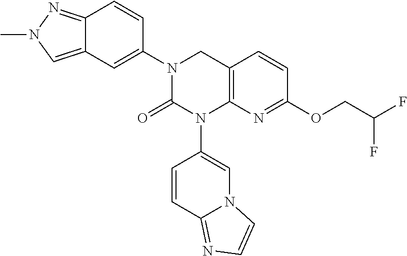 [SMILES: Cn1cc2cc(N3Cc4ccc(OCC(F)F)nc4N(c4ccc5nccn5c4)C3=O)ccc2n1] |
| 172 | 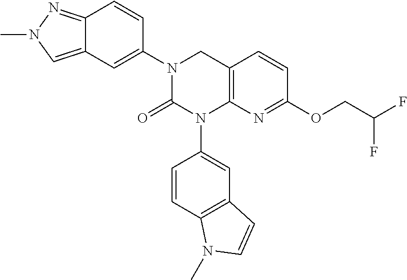 [SMILES: Cn1cc2cc(N3Cc4ccc(OCC(F)F)nc4N(c4ccc5c(ccn5C)c4)C3=O)ccc2n1] |
| 173 | 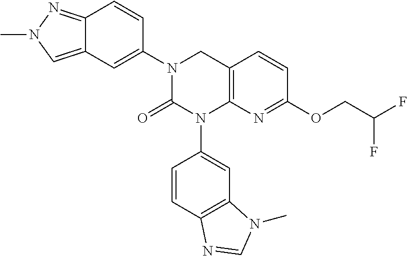 [SMILES: Cn1cc2cc(N3Cc4ccc(OCC(F)F)nc4N(c4ccc5ncn(C)c5c4)C3=O)ccc2n1] |
| 174 | 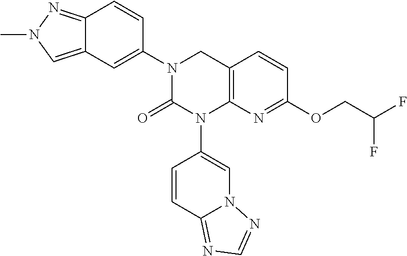 [SMILES: Cn1cc2cc(N3Cc4ccc(OCC(F)F)nc4N(c4ccc5ncnn5c4)C3=O)ccc2n1] |
| 175 | 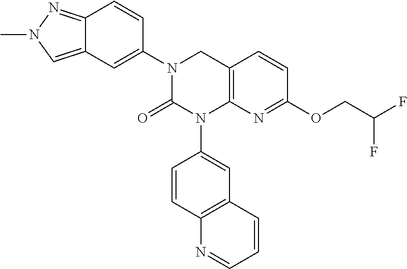 [SMILES: Cn1cc2cc(N3Cc4ccc(OCC(F)F)nc4N(c4ccc5ncccc5c4)C3=O)ccc2n1] |
| 176 | 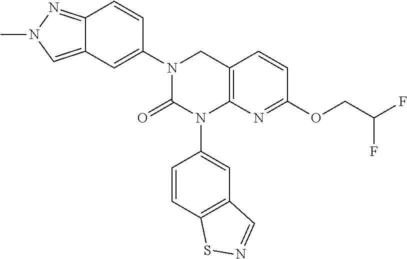 [SMILES: Cn1cc2cc(N3Cc4ccc(OCC(F)F)nc4N(c4ccc5sncc5c4)C3=O)ccc2n1] |
| 177 | 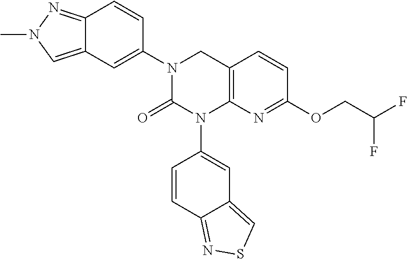 [SMILES: Cn1cc2cc(N3Cc4ccc(OCC(F)F)nc4N(c4ccc5nscc5c4)C3=O)ccc2n1] |
| 178 | 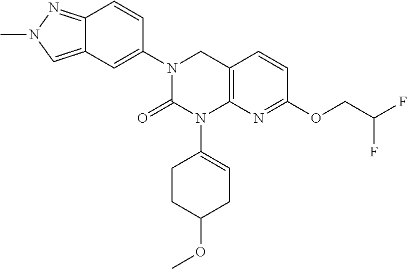 [SMILES: COC1CC=C(N2C(=O)N(c3ccc4nn(C)cc4c3)Cc3ccc(OCC(F)F)nc32)CC1] |
| 179 | 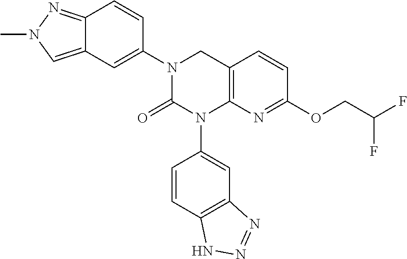 [SMILES: Cn1cc2cc(N3Cc4ccc(OCC(F)F)nc4N(c4ccc5[nH]nnc5c4)C3=O)ccc2n1] |
| 180 | 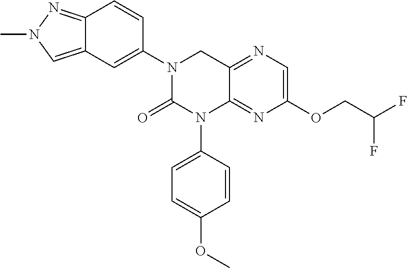 [SMILES: COc1ccc(N2C(=O)N(c3ccc4nn(C)cc4c3)Cc3ncc(OCC(F)F)nc32)cc1] |
| 181 | 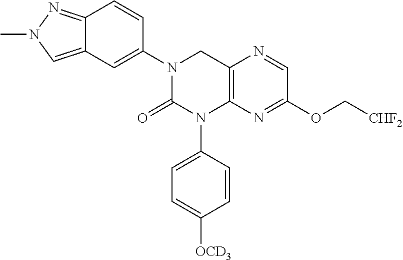 |
| 182 | 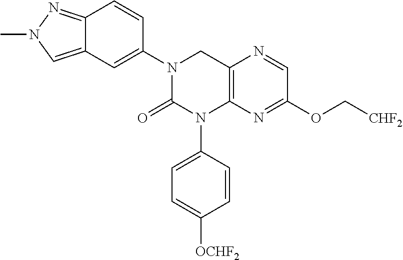 [SMILES: Cn1cc2cc(N3Cc4ncc(OCC(F)F)nc4N(c4ccc(OC(F)F)cc4)C3=O)ccc2n1] |
| 183 | 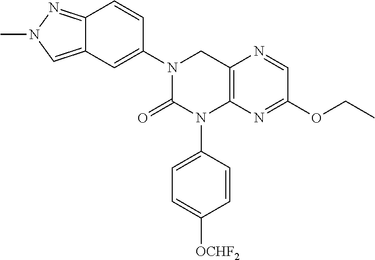 [SMILES: CCOc1cnc2c(n1)N(c1ccc(OC(F)F)cc1)C(=O)N(c1ccc3nn(C)cc3c1)C2] |
| 184 | 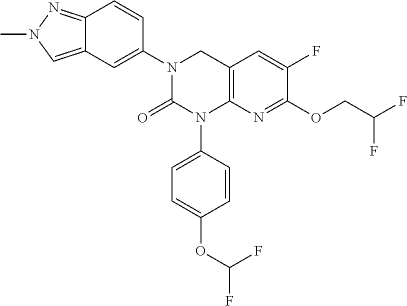 [SMILES: Cn1cc2cc(N3Cc4cc(F)c(OCC(F)F)nc4N(c4ccc(OC(F)F)cc4)C3=O)ccc2n1] |
| 185 | 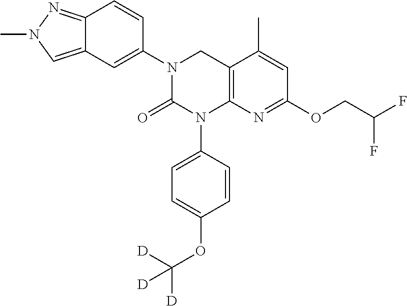 [SMILES: [2H]C([2H])([2H])Oc1ccc(N2C(=O)N(c3ccc4nn(C)cc4c3)Cc3c(C)cc(OCC(F)F)nc32)cc1] |
| 186 | 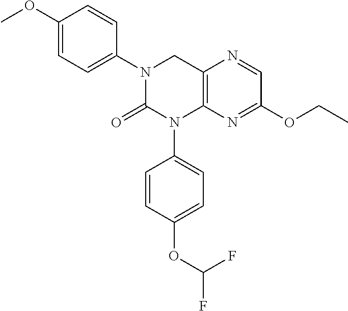 [SMILES: CCOc1cnc2c(n1)N(c1ccc(OC(F)F)cc1)C(=O)N(c1ccc(OC)cc1)C2] |
| 187 | 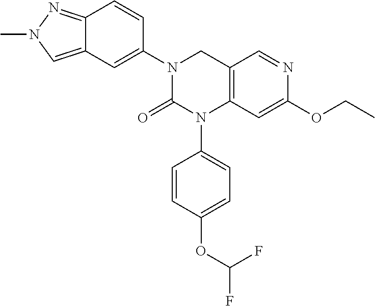 [SMILES: CCOc1cc2c(cn1)CN(c1ccc3nn(C)cc3c1)C(=O)N2c1ccc(OC(F)F)cc1] |
| 188 | 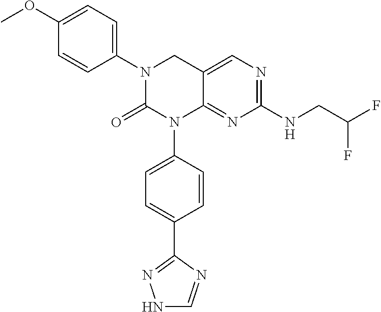 [SMILES: COc1ccc(N2Cc3cnc(NCC(F)F)nc3N(c3ccc(-c4nc[nH]n4)cc3)C2=O)cc1] |
| 189 | 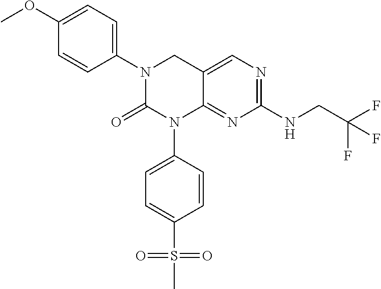 [SMILES: COc1ccc(N2Cc3cnc(NCC(F)(F)F)nc3N(c3ccc(S(C)(=O)=O)cc3)C2=O)cc1] |
| 190 | 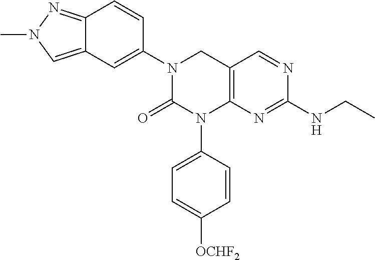 [SMILES: CCNc1ncc2c(n1)N(c1ccc(OC(F)F)cc1)C(=O)N(c1ccc3nn(C)cc3c1)C2] |
| 191 | 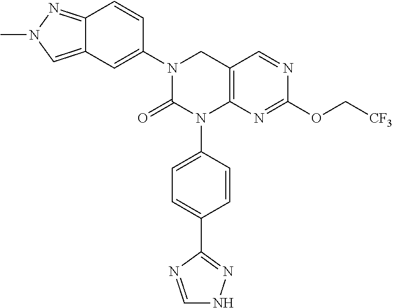 [SMILES: Cn1cc2cc(N3Cc4cnc(OCC(F)(F)F)nc4N(c4ccc(-c5nc[nH]n5)cc4)C3=O)ccc2n1] |
| 192 | 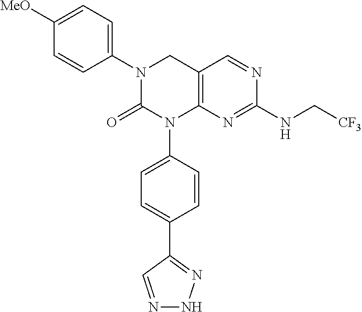 [SMILES: COc1ccc(N2Cc3cnc(NCC(F)(F)F)nc3N(c3ccc(-c4cn[nH]n4)cc3)C2=O)cc1] |
| 193 | 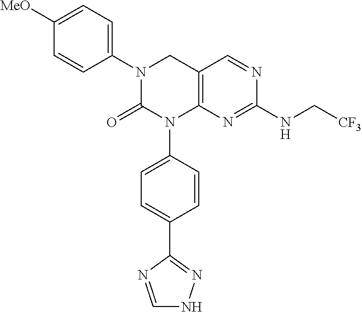 [SMILES: COc1ccc(N2Cc3cnc(NCC(F)(F)F)nc3N(c3ccc(-c4nc[nH]n4)cc3)C2=O)cc1] |
| 194 | 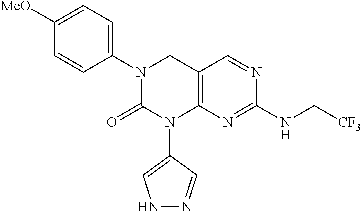 [SMILES: COc1ccc(N2Cc3cnc(NCC(F)(F)F)nc3N(c3cn[nH]c3)C2=O)cc1] |
| 195 | 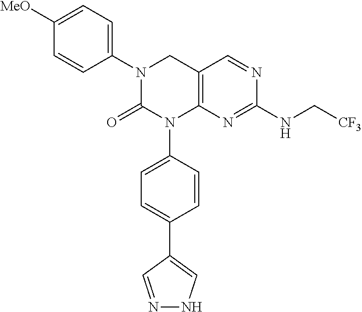 [SMILES: COc1ccc(N2Cc3cnc(NCC(F)(F)F)nc3N(c3ccc(-c4cn[nH]c4)cc3)C2=O)cc1] |
| 196 | 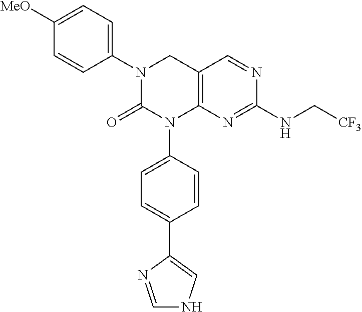 [SMILES: COc1ccc(N2Cc3cnc(NCC(F)(F)F)nc3N(c3ccc(-c4c[nH]cn4)cc3)C2=O)cc1] |
| 197 | 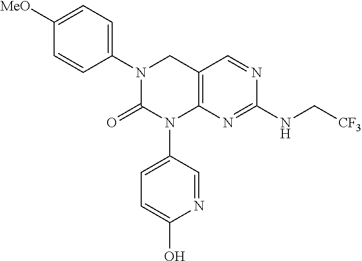 [SMILES: COc1ccc(N2Cc3cnc(NCC(F)(F)F)nc3N(c3ccc(O)nc3)C2=O)cc1] |
| 198 | 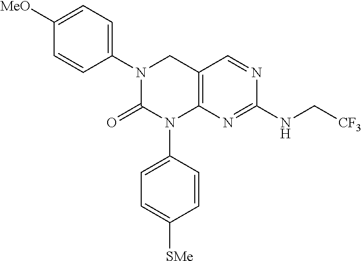 [SMILES: COc1ccc(N2Cc3cnc(NCC(F)(F)F)nc3N(c3ccc(SC)cc3)C2=O)cc1] |
| 199 | 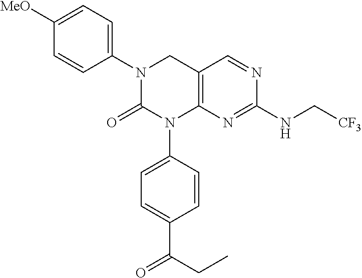 [SMILES: CCC(=O)c1ccc(N2C(=O)N(c3ccc(OC)cc3)Cc3cnc(NCC(F)(F)F)nc32)cc1] |
| 200 | 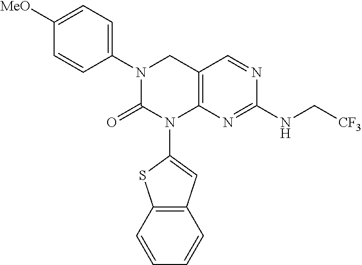 [SMILES: COc1ccc(N2Cc3cnc(NCC(F)(F)F)nc3N(c3cc4ccccc4s3)C2=O)cc1] |
| 201 | 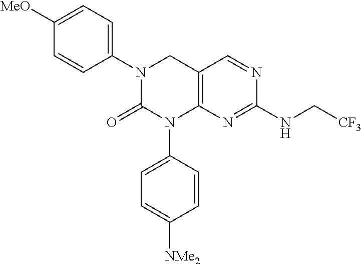 [SMILES: COc1ccc(N2Cc3cnc(NCC(F)(F)F)nc3N(c3ccc(N(C)C)cc3)C2=O)cc1] |
| 202 | 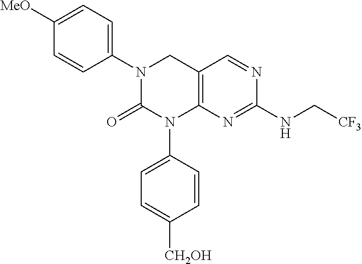 [SMILES: COc1ccc(N2Cc3cnc(NCC(F)(F)F)nc3N(c3ccc(CO)cc3)C2=O)cc1] |
| 203 | 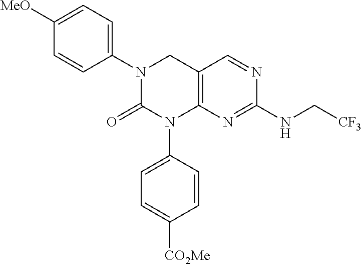 [SMILES: COC(=O)c1ccc(N2C(=O)N(c3ccc(OC)cc3)Cc3cnc(NCC(F)(F)F)nc32)cc1] |
| 204 | 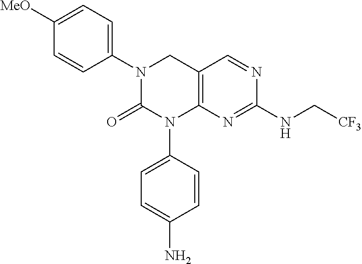 [SMILES: COc1ccc(N2Cc3cnc(NCC(F)(F)F)nc3N(c3ccc(N)cc3)C2=O)cc1] |
| 205 | 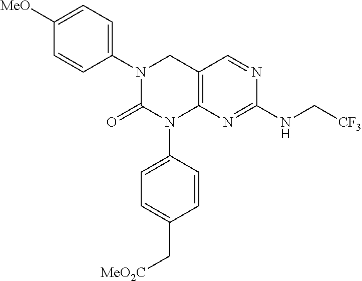 [SMILES: COC(=O)Cc1ccc(N2C(=O)N(c3ccc(OC)cc3)Cc3cnc(NCC(F)(F)F)nc32)cc1] |
| 206 | 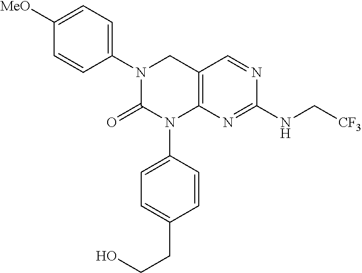 [SMILES: COc1ccc(N2Cc3cnc(NCC(F)(F)F)nc3N(c3ccc(CCO)cc3)C2=O)cc1] |
| 207 | 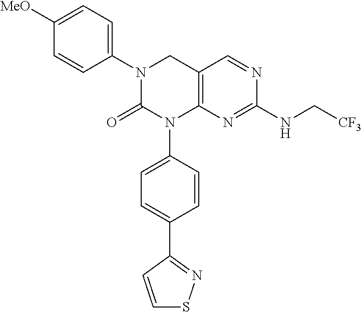 [SMILES: COc1ccc(N2Cc3cnc(NCC(F)(F)F)nc3N(c3ccc(-c4ccsn4)cc3)C2=O)cc1] |
| 208 | 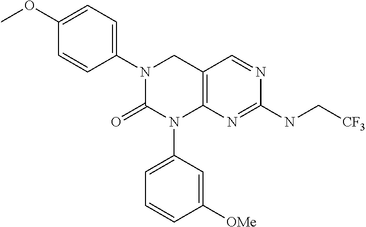 [SMILES: COc1ccc(N2Cc3cnc(NCC(F)(F)F)nc3N(c3cccc(OC)c3)C2=O)cc1] |
| 209 | 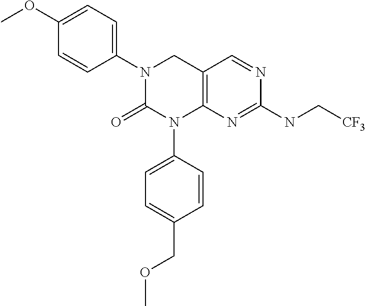 [SMILES: COCc1ccc(N2C(=O)N(c3ccc(OC)cc3)Cc3cnc(NCC(F)(F)F)nc32)cc1] |
| 210 | 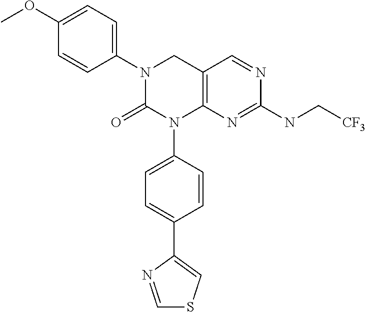 [SMILES: COc1ccc(N2Cc3cnc(NCC(F)(F)F)nc3N(c3ccc(-c4cscn4)cc3)C2=O)cc1] |
| 211 | 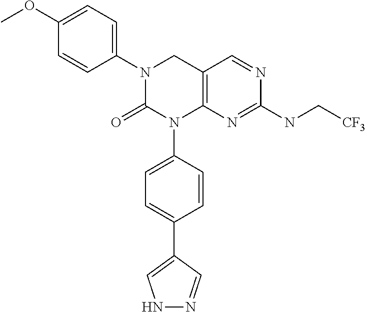 [SMILES: COc1ccc(N2Cc3cnc(NCC(F)(F)F)nc3N(c3ccc(-c4cn[nH]c4)cc3)C2=O)cc1] |
| 212 | 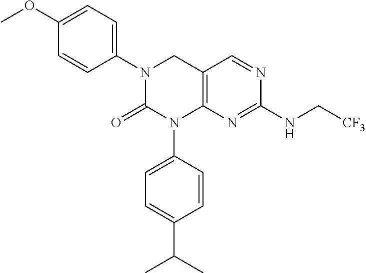 [SMILES: COc1ccc(N2Cc3cnc(NCC(F)(F)F)nc3N(c3ccc(C(C)C)cc3)C2=O)cc1] |
| 213 | 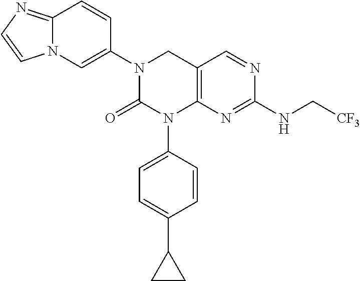 [SMILES: O=C1N(c2ccc3nccn3c2)Cc2cnc(NCC(F)(F)F)nc2N1c1ccc(C2CC2)cc1] |
| 214 | 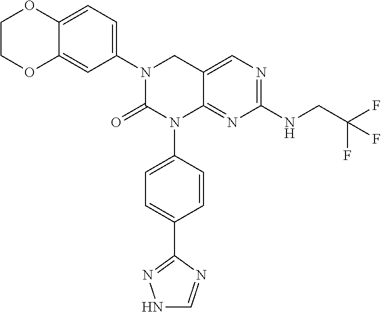 [SMILES: O=C1N(c2ccc3c(c2)OCCO3)Cc2cnc(NCC(F)(F)F)nc2N1c1ccc(-c2nc[nH]n2)cc1] |
| 215 | 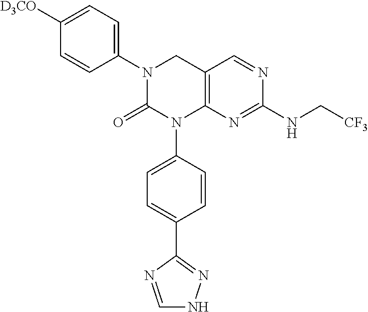 |
| 216 | 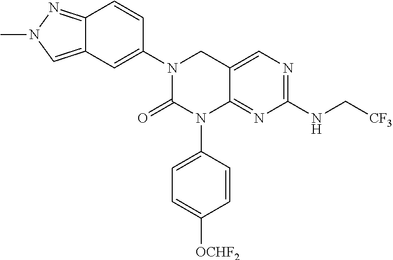 [SMILES: Cn1cc2cc(N3Cc4cnc(NCC(F)(F)F)nc4N(c4ccc(OC(F)F)cc4)C3=O)ccc2n1] |
| 217 | 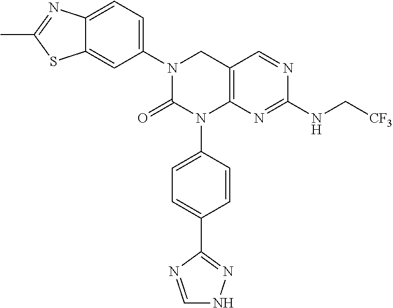 [SMILES: Cc1nc2ccc(N3Cc4cnc(NCC(F)(F)F)nc4N(c4ccc(-c5nc[nH]n5)cc4)C3=O)cc2s1] |
| 218 | 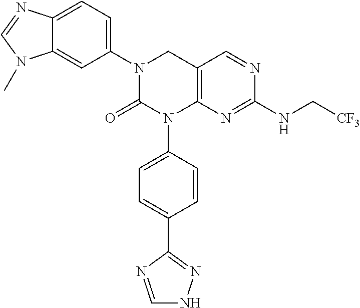 [SMILES: Cn1cnc2ccc(N3Cc4cnc(NCC(F)(F)F)nc4N(c4ccc(-c5nc[nH]n5)cc4)C3=O)cc21] |
| 219 | 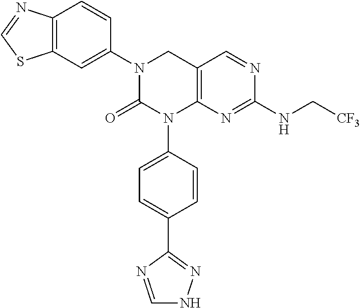 [SMILES: O=C1N(c2ccc3ncsc3c2)Cc2cnc(NCC(F)(F)F)nc2N1c1ccc(-c2nc[nH]n2)cc1] |
| 220 | 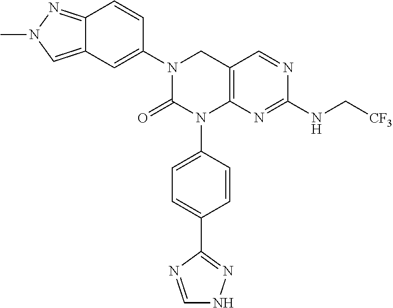 [SMILES: Cn1cc2cc(N3Cc4cnc(NCC(F)(F)F)nc4N(c4ccc(-c5nc[nH]n5)cc4)C3=O)ccc2n1] |
| 221 | 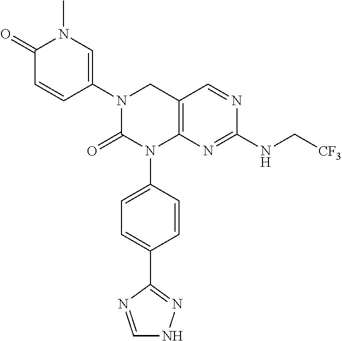 [SMILES: Cn1cc(N2Cc3cnc(NCC(F)(F)F)nc3N(c3ccc(-c4nc[nH]n4)cc3)C2=O)ccc1=O] |
| 222 | 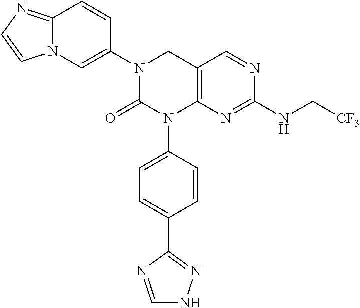 [SMILES: O=C1N(c2ccc3nccn3c2)Cc2cnc(NCC(F)(F)F)nc2N1c1ccc(-c2nc[nH]n2)cc1] |
| 223 | 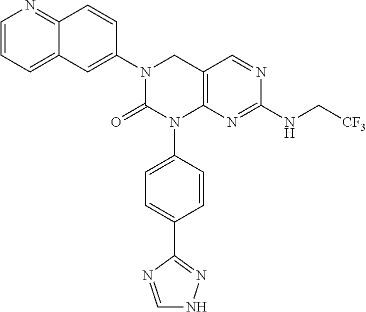 [SMILES: O=C1N(c2ccc3ncccc3c2)Cc2cnc(NCC(F)(F)F)nc2N1c1ccc(-c2nc[nH]n2)cc1] |
| 224 | 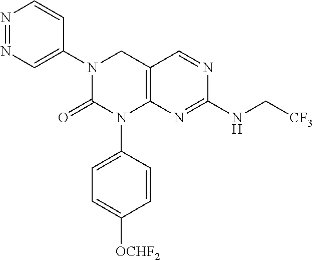 [SMILES: O=C1N(c2ccnnc2)Cc2cnc(NCC(F)(F)F)nc2N1c1ccc(OC(F)F)cc1] |
| 225 | 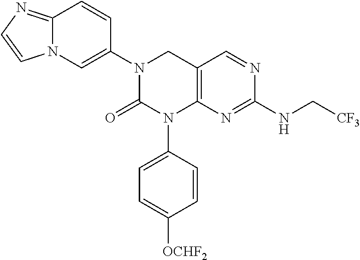 [SMILES: O=C1N(c2ccc3nccn3c2)Cc2cnc(NCC(F)(F)F)nc2N1c1ccc(OC(F)F)cc1] |
| 226 | 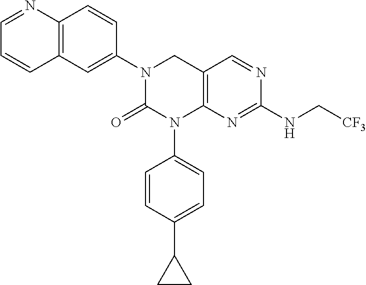 [SMILES: O=C1N(c2ccc3ncccc3c2)Cc2cnc(NCC(F)(F)F)nc2N1c1ccc(C2CC2)cc1] |
| 227 | 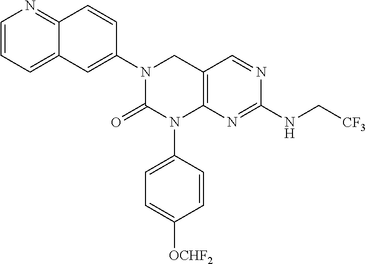 [SMILES: O=C1N(c2ccc3ncccc3c2)Cc2cnc(NCC(F)(F)F)nc2N1c1ccc(OC(F)F)cc1] |
| 228 | 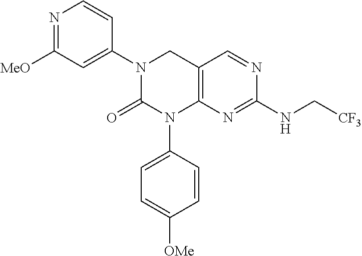 [SMILES: COc1ccc(N2C(=O)N(c3ccnc(OC)c3)Cc3cnc(NCC(F)(F)F)nc32)cc1] |
| 229 | 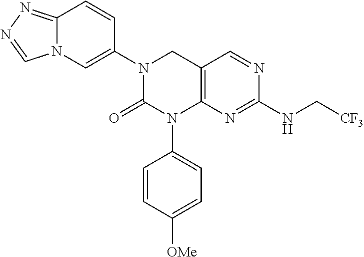 [SMILES: COc1ccc(N2C(=O)N(c3ccc4nncn4c3)Cc3cnc(NCC(F)(F)F)nc32)cc1] |
| 230 | 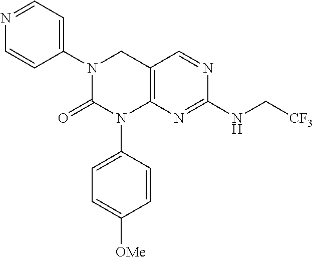 [SMILES: COc1ccc(N2C(=O)N(c3ccncc3)Cc3cnc(NCC(F)(F)F)nc32)cc1] |
| 231 | 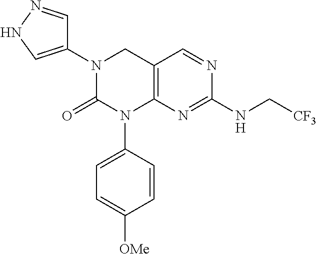 [SMILES: COc1ccc(N2C(=O)N(c3cn[nH]c3)Cc3cnc(NCC(F)(F)F)nc32)cc1] |
| 232 | 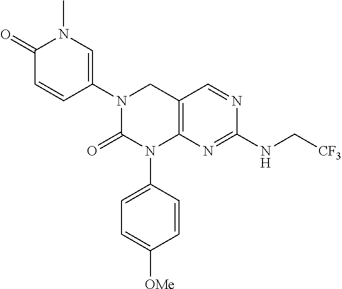 [SMILES: COc1ccc(N2C(=O)N(c3ccc(=O)n(C)c3)Cc3cnc(NCC(F)(F)F)nc32)cc1] |
| 233 | 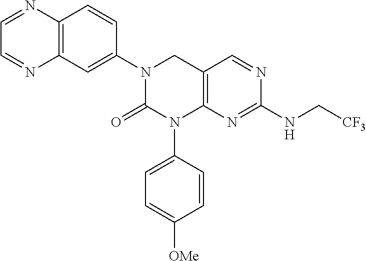 [SMILES: COc1ccc(N2C(=O)N(c3ccc4nccnc4c3)Cc3cnc(NCC(F)(F)F)nc32)cc1] |
| 234 | 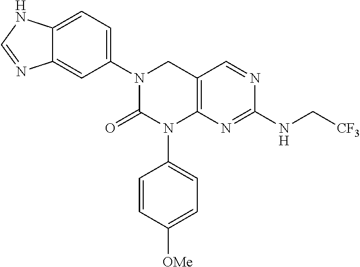 [SMILES: COc1ccc(N2C(=O)N(c3ccc4[nH]cnc4c3)Cc3cnc(NCC(F)(F)F)nc32)cc1] |
| 235 | 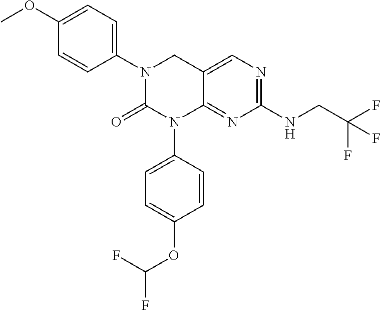 [SMILES: COc1ccc(N2Cc3cnc(NCC(F)(F)F)nc3N(c3ccc(OC(F)F)cc3)C2=O)cc1] |
| 236 | 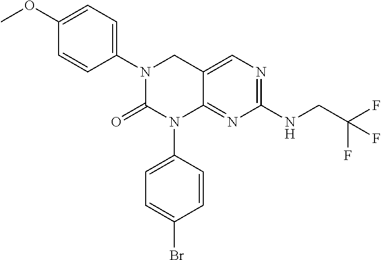 [SMILES: COc1ccc(N2Cc3cnc(NCC(F)(F)F)nc3N(c3ccc(Br)cc3)C2=O)cc1] |
| 237 | 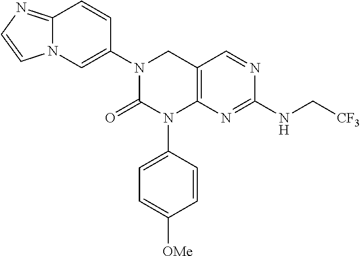 [SMILES: COc1ccc(N2C(=O)N(c3ccc4nccn4c3)Cc3cnc(NCC(F)(F)F)nc32)cc1] |
| 238 | 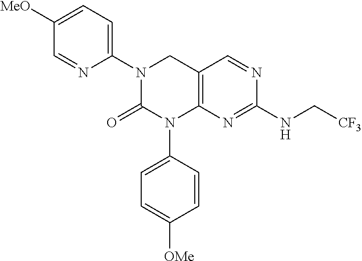 [SMILES: COc1ccc(N2C(=O)N(c3ccc(OC)cn3)Cc3cnc(NCC(F)(F)F)nc32)cc1] |
| 239 | 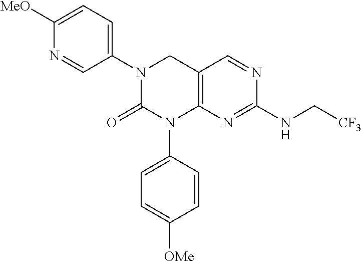 [SMILES: COc1ccc(N2C(=O)N(c3ccc(OC)nc3)Cc3cnc(NCC(F)(F)F)nc32)cc1] |
| 240 | 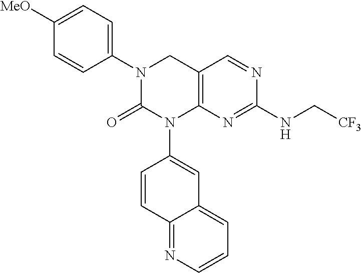 [SMILES: COc1ccc(N2Cc3cnc(NCC(F)(F)F)nc3N(c3ccc4ncccc4c3)C2=O)cc1] |
| 241 | 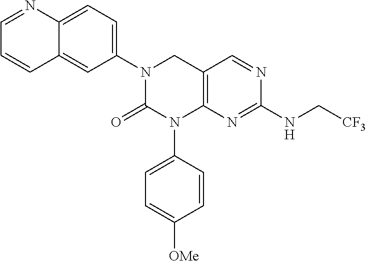 [SMILES: COc1ccc(N2C(=O)N(c3ccc4ncccc4c3)Cc3cnc(NCC(F)(F)F)nc32)cc1] |
| 242 | 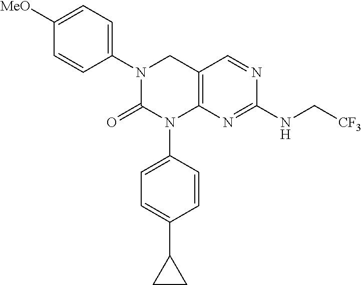 [SMILES: COc1ccc(N2Cc3cnc(NCC(F)(F)F)nc3N(c3ccc(C4CC4)cc3)C2=O)cc1] |
| 243 | 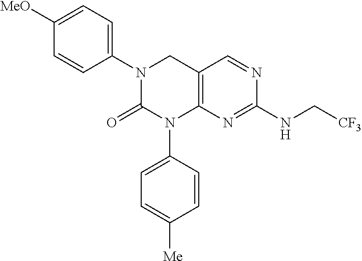 [SMILES: COc1ccc(N2Cc3cnc(NCC(F)(F)F)nc3N(c3ccc(C)cc3)C2=O)cc1] |
| 244 | 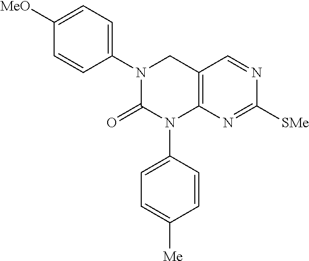 [SMILES: COc1ccc(N2Cc3cnc(SC)nc3N(c3ccc(C)cc3)C2=O)cc1] |
| 245 | 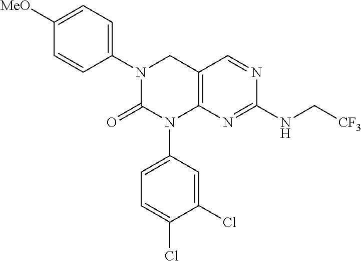 [SMILES: COc1ccc(N2Cc3cnc(NCC(F)(F)F)nc3N(c3ccc(Cl)c(Cl)c3)C2=O)cc1] |
| 246 | 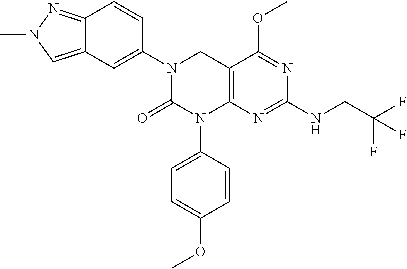 [SMILES: COc1ccc(N2C(=O)N(c3ccc4nn(C)cc4c3)Cc3c(OC)nc(NCC(F)(F)F)nc32)cc1] |
| 247 | 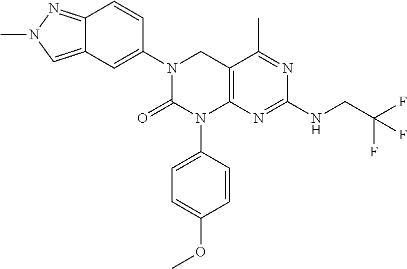 [SMILES: COc1ccc(N2C(=O)N(c3ccc4nn(C)cc4c3)Cc3c(C)nc(NCC(F)(F)F)nc32)cc1] |
| 248 | 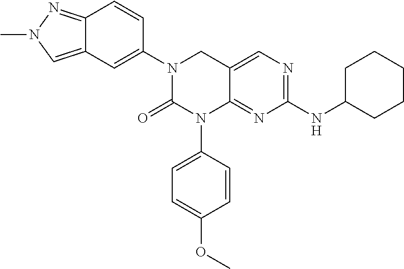 [SMILES: COc1ccc(N2C(=O)N(c3ccc4nn(C)cc4c3)Cc3cnc(NC4CCCCC4)nc32)cc1] |
| 249 | 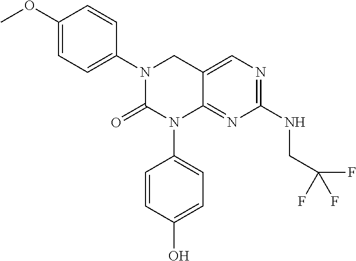 [SMILES: COc1ccc(N2Cc3cnc(NCC(F)(F)F)nc3N(c3ccc(O)cc3)C2=O)cc1] |
| 250 | 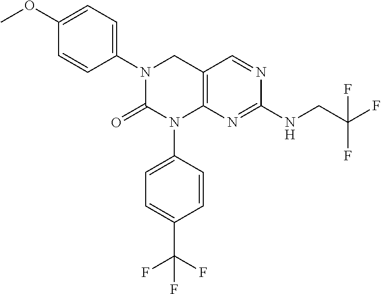 [SMILES: COc1ccc(N2Cc3cnc(NCC(F)(F)F)nc3N(c3ccc(C(F)(F)F)cc3)C2=O)cc1] |
| 251 | 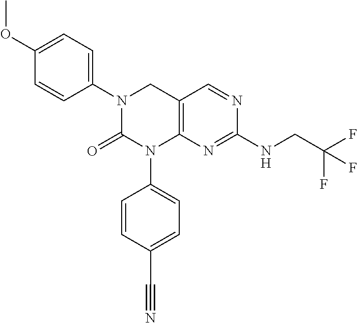 [SMILES: COc1ccc(N2Cc3cnc(NCC(F)(F)F)nc3N(c3ccc(C#N)cc3)C2=O)cc1] |
| 252 | 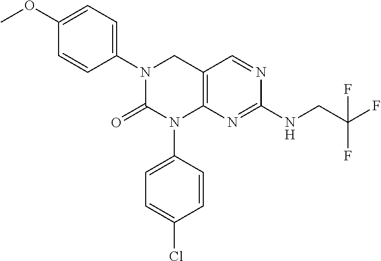 [SMILES: COc1ccc(N2Cc3cnc(NCC(F)(F)F)nc3N(c3ccc(Cl)cc3)C2=O)cc1] |
| 253 | 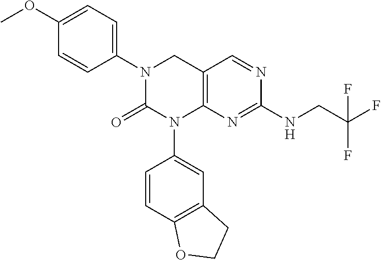 [SMILES: COc1ccc(N2Cc3cnc(NCC(F)(F)F)nc3N(c3ccc4c(c3)CCO4)C2=O)cc1] |
| 254 | 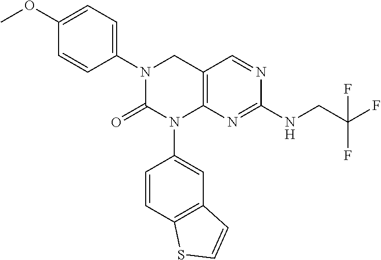 [SMILES: COc1ccc(N2Cc3cnc(NCC(F)(F)F)nc3N(c3ccc4sccc4c3)C2=O)cc1] |
| 255 | 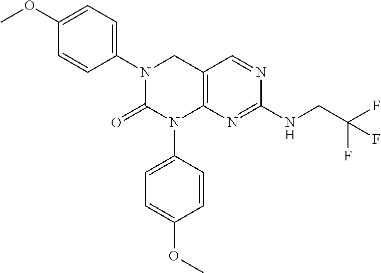 [SMILES: COc1ccc(N2Cc3cnc(NCC(F)(F)F)nc3N(c3ccc(OC)cc3)C2=O)cc1] |
| 256 | 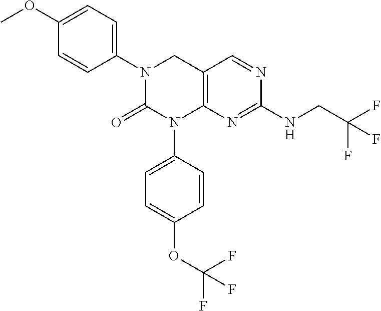 [SMILES: COc1ccc(N2Cc3cnc(NCC(F)(F)F)nc3N(c3ccc(OC(F)(F)F)cc3)C2=O)cc1] |
| 257 | 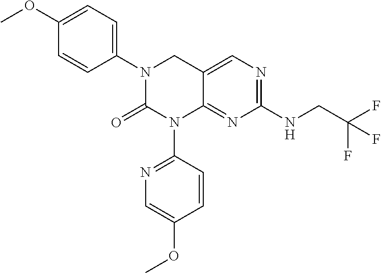 [SMILES: COc1ccc(N2Cc3cnc(NCC(F)(F)F)nc3N(c3ccc(OC)cn3)C2=O)cc1] |
| 258 | 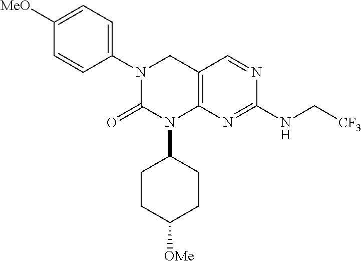 |
| 259 | 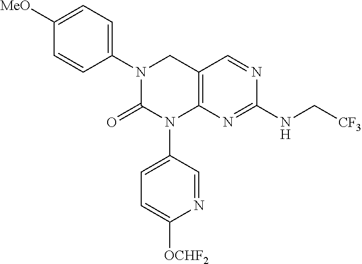 [SMILES: COc1ccc(N2Cc3cnc(NCC(F)(F)F)nc3N(c3ccc(OC(F)F)nc3)C2=O)cc1] |
| 260 | 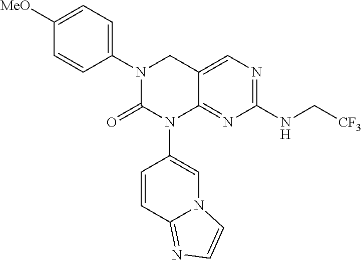 [SMILES: COc1ccc(N2Cc3cnc(NCC(F)(F)F)nc3N(c3ccc4nccn4c3)C2=O)cc1] |
| 261 | 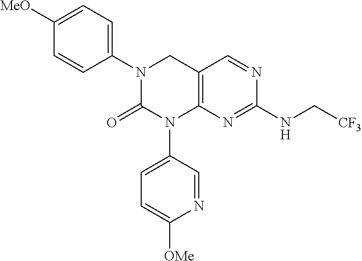 [SMILES: COc1ccc(N2Cc3cnc(NCC(F)(F)F)nc3N(c3ccc(OC)nc3)C2=O)cc1] |
| 262 | 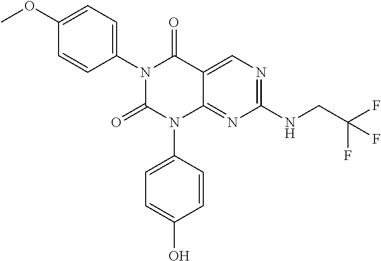 [SMILES: COc1ccc(-n2c(=O)c3cnc(NCC(F)(F)F)nc3n(-c3ccc(O)cc3)c2=O)cc1] |
| 263 | 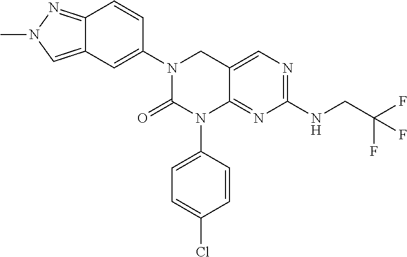 [SMILES: Cn1cc2cc(N3Cc4cnc(NCC(F)(F)F)nc4N(c4ccc(Cl)cc4)C3=O)ccc2n1] |
| 264 | 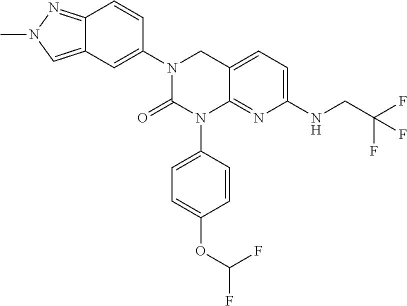 [SMILES: Cn1cc2cc(N3Cc4ccc(NCC(F)(F)F)nc4N(c4ccc(OC(F)F)cc4)C3=O)ccc2n1] |
| 265 | 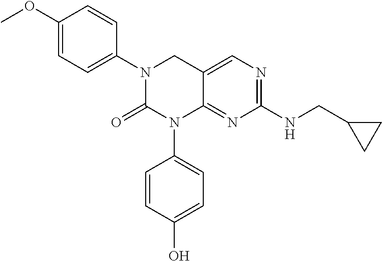 [SMILES: COc1ccc(N2Cc3cnc(NCC4CC4)nc3N(c3ccc(O)cc3)C2=O)cc1] |
| 266 | 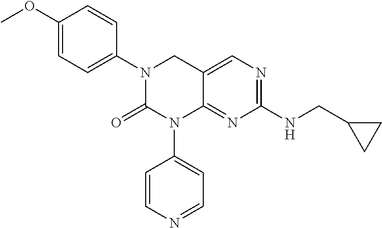 [SMILES: COc1ccc(N2Cc3cnc(NCC4CC4)nc3N(c3ccncc3)C2=O)cc1] |
| 267 | 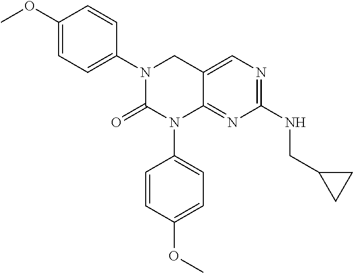 [SMILES: COc1ccc(N2Cc3cnc(NCC4CC4)nc3N(c3ccc(OC)cc3)C2=O)cc1] |
| 269 | 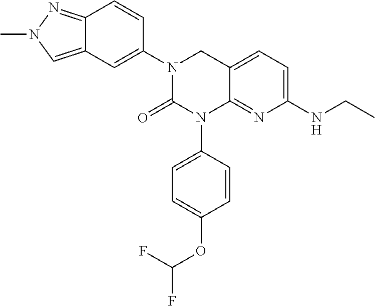 [SMILES: CCNc1ccc2c(n1)N(c1ccc(OC(F)F)cc1)C(=O)N(c1ccc3nn(C)cc3c1)C2] |
| 270 | 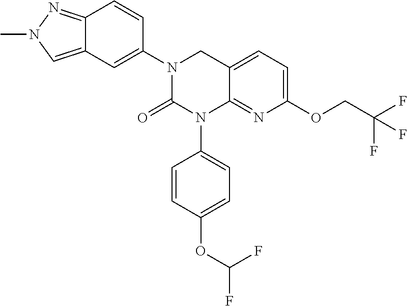 [SMILES: Cn1cc2cc(N3Cc4ccc(OCC(F)(F)F)nc4N(c4ccc(OC(F)F)cc4)C3=O)ccc2n1] |
| 271 | 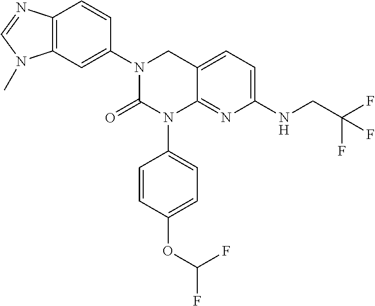 [SMILES: Cn1cnc2ccc(N3Cc4ccc(NCC(F)(F)F)nc4N(c4ccc(OC(F)F)cc4)C3=O)cc21] |
| 272 | 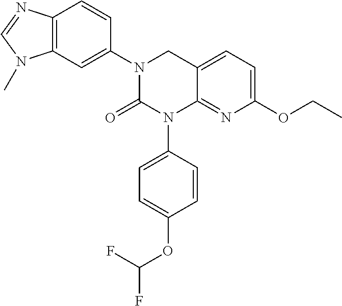 [SMILES: CCOc1ccc2c(n1)N(c1ccc(OC(F)F)cc1)C(=O)N(c1ccc3ncn(C)c3c1)C2] |
| 273 | 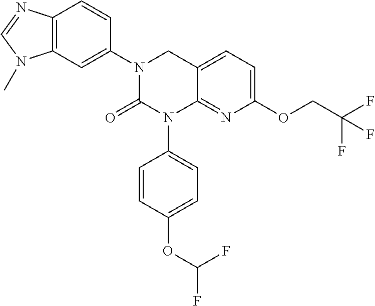 [SMILES: Cn1cnc2ccc(N3Cc4ccc(OCC(F)(F)F)nc4N(c4ccc(OC(F)F)cc4)C3=O)cc21] |
| 274 | 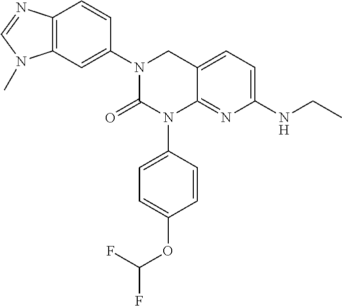 [SMILES: CCNc1ccc2c(n1)N(c1ccc(OC(F)F)cc1)C(=O)N(c1ccc3ncn(C)c3c1)C2] |
| 275 | 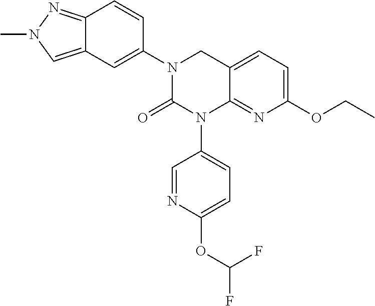 [SMILES: CCOc1ccc2c(n1)N(c1ccc(OC(F)F)nc1)C(=O)N(c1ccc3nn(C)cc3c1)C2] |
| 276 | 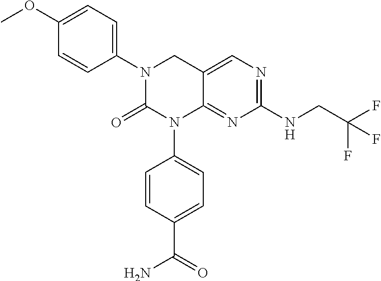 [SMILES: COc1ccc(N2Cc3cnc(NCC(F)(F)F)nc3N(c3ccc(C(N)=O)cc3)C2=O)cc1] |
| 277 | 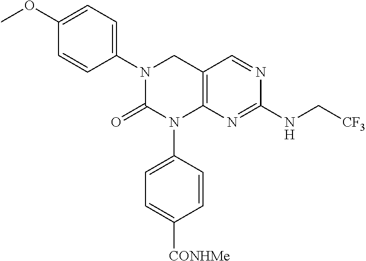 |
| 278 | 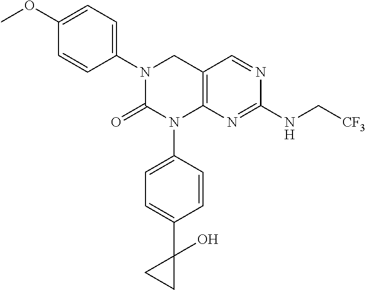 [SMILES: COc1ccc(N2Cc3cnc(NCC(F)(F)F)nc3N(c3ccc(C4(O)CC4)cc3)C2=O)cc1] |
| 279 | 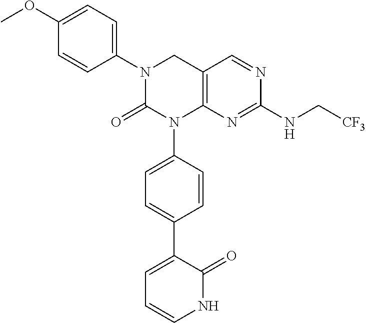 [SMILES: COc1ccc(N2Cc3cnc(NCC(F)(F)F)nc3N(c3ccc(-c4ccc[nH]c4=O)cc3)C2=O)cc1] |
| 280 | 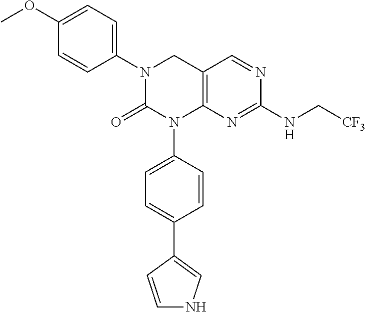 [SMILES: COc1ccc(N2Cc3cnc(NCC(F)(F)F)nc3N(c3ccc(-c4cc[nH]c4)cc3)C2=O)cc1] |
| 281 | 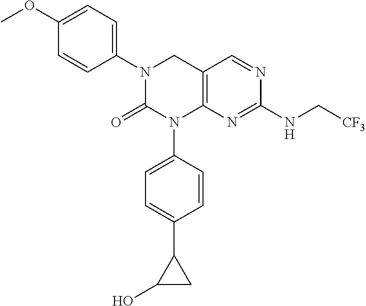 [SMILES: COc1ccc(N2Cc3cnc(NCC(F)(F)F)nc3N(c3ccc(C4CC4O)cc3)C2=O)cc1] |
| 282 | 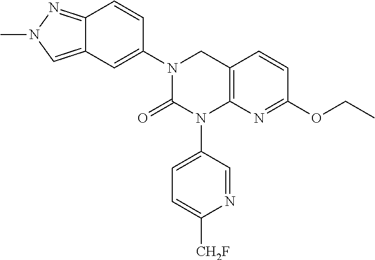 |
| 283 | 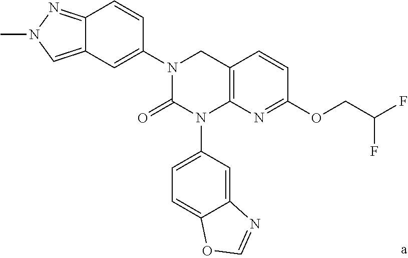 [SMILES: Cn1cc2cc(N3Cc4ccc(OCC(F)F)nc4N(c4ccc5ocnc5c4)C3=O)ccc2n1] |
| 286 | 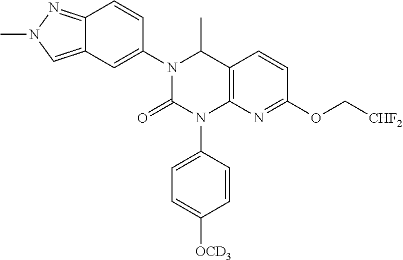 |
| 294 | 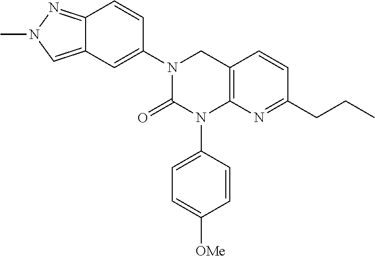 [SMILES: CCCc1ccc2c(n1)N(c1ccc(OC)cc1)C(=O)N(c1ccc3nn(C)cc3c1)C2] |
| 295 | 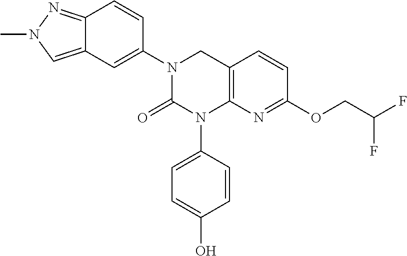 [SMILES: Cn1cc2cc(N3Cc4ccc(OCC(F)F)nc4N(c4ccc(O)cc4)C3=O)ccc2n1] |

Aspect 36. The compound according to Aspect 2, wherein the compound is selected from the following table:

| | |
|---|---|
| 268 | 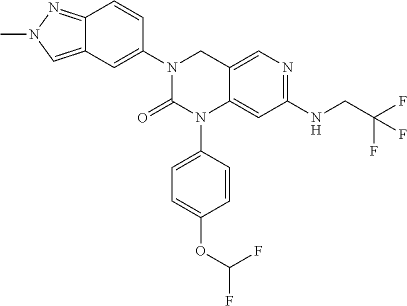 [SMILES: Cn1cc2cc(N3Cc4cnc(NCC(F)(F)F)cc4N(c4ccc(OC(F)F)cc4)C3=O)ccc2n1] |
| 284 | 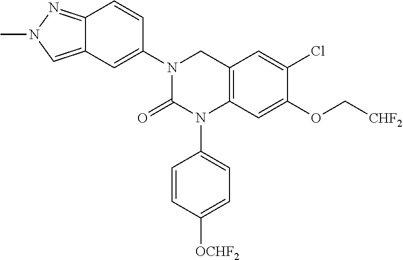 [SMILES: Cn1cc2cc(N3Cc4cc(Cl)c(OCC(F)F)cc4N(c4ccc(OC(F)F)cc4)C3=O)ccc2n1] |
| 285 | 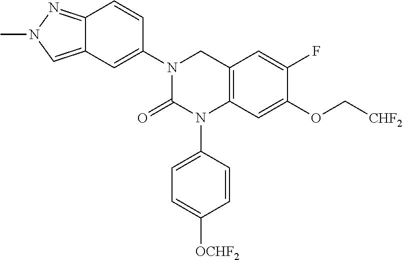 [SMILES: Cn1cc2cc(N3Cc4cc(F)c(OCC(F)F)cc4N(c4ccc(OC(F)F)cc4)C3=O)ccc2n1] |
| 287 | 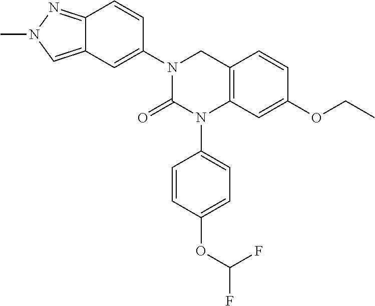 [SMILES: CCOc1ccc2c(c1)N(c1ccc(OC(F)F)cc1)C(=O)N(c1ccc3nn(C)cc3c1)C2] |
| 288 | 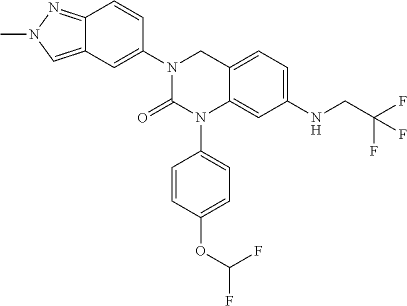 [SMILES: Cn1cc2cc(N3Cc4ccc(NCC(F)(F)F)cc4N(c4ccc(OC(F)F)cc4)C3=O)ccc2n1] |
| 289 | 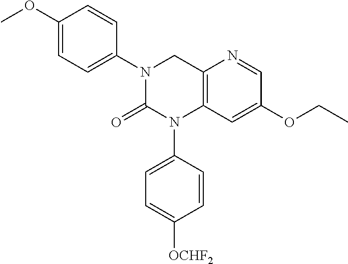 [SMILES: CCOc1cnc2c(c1)N(c1ccc(OC(F)F)cc1)C(=O)N(c1ccc(OC)cc1)C2] |
| 290 | 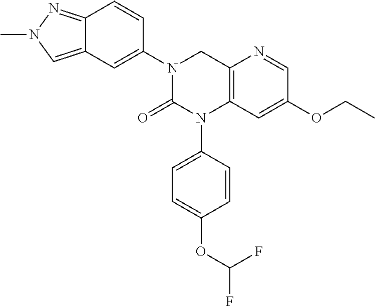 [SMILES: CCOc1cnc2c(c1)N(c1ccc(OC(F)F)cc1)C(=O)N(c1ccc3nn(C)cc3c1)C2] |
| 291 | 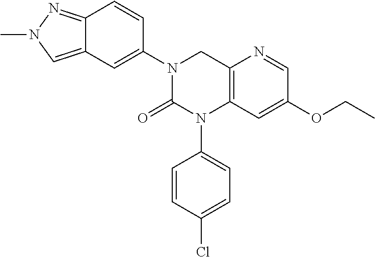 [SMILES: CCOc1cnc2c(c1)N(c1ccc(Cl)cc1)C(=O)N(c1ccc3nn(C)cc3c1)C2] |
| 292 | 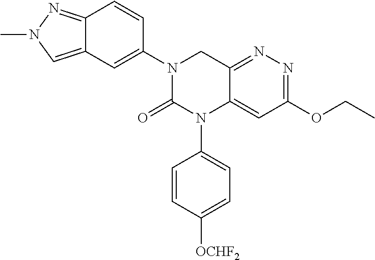 [SMILES: CCOc1cc2c(nn1)CN(c1ccc3nn(C)cc3c1)C(=O)N2c1ccc(OC(F)F)cc1] |
| 293 | 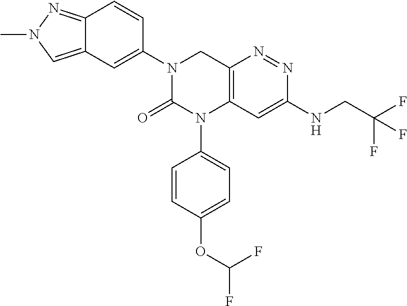 [SMILES: Cn1cc2cc(N3Cc4nnc(NCC(F)(F)F)cc4N(c4ccc(OC(F)F)cc4)C3=O)ccc2n1] |

Aspect 37. A pharmaceutical composition comprising a therapeutically effective amount of a compound according to any one of Aspects 1 to 36 or a pharmaceutically acceptable salt thereof, and a pharmaceutically acceptable carrier.

Aspect 38. A method for treating a cancer in a subject suffering therefrom, comprising administering to the subject an effective amount of a MAT2A inhibitor compound, or a pharmaceutically acceptable salt thereof, according to any one of Aspects 1-36.

Aspect 39. The method according to Aspect 38, wherein the cancer is an MTAP-deleted cancer.

Aspect 40. A method for inhibiting the synthesis of S-adenosyl methionine (SAM) in a cell, comprising introducing into the cell an effective amount of a compound, or a pharmaceutically acceptable salt thereof, according to any one of Aspects 1 to 36.

Aspect 41. The method according to Aspect 40, wherein the cell is in a subject.

Aspect 42. A method for inhibiting the synthesis of S-adenosyl methionine (SAM) in a subject, comprising administering to the subject an effective amount of at least one compound or a salt thereof according to any one of Aspects 1 to 36.

Aspect 43. A method for treating a cancer in a subject suffering therefrom, comprising administering to the subject an effective amount of a compound according to any one of Aspects 1 to 36.

Aspect 44. The method according to Aspect 43, wherein the cancer is an MTAP-deleted cancer.

Aspect 45. The method according to Aspect 38, 39, 43, or 44, wherein the cancer is selected from the group consisting of mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

Aspect 46. The method according to Aspect 43 or 44, wherein the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, and breast cancer.

Aspect 47. The method according to Aspect 46, wherein the cancer is a lung cancer is selected from the group consisting of non-small cell lung cancer, small cell lung cancer, adenocarcinoma of the lung, and squamous cell carcinoma of the lung.

Aspect 48. The method according to Aspect 46, wherein the cancer is a brain tumor selected from the group consisting of glioma, glioblastoma, astrocytoma, meningioma, medulloblastoma, peripheral neuroectodermal tumors, and craniopharyngioma.

Aspect 49. The method according to Aspect 46, wherein the cancer is triple negative breast cancer (TNBC).

Aspect 50. The method according to Aspect 46, wherein the cancer is a lymphoma selected from the group consisting of mantle cell lymphoma, Hodgkin lymphoma, non-Hodgkin lymphoma, Burkitt lymphoma, diffuse large B-cell lymphoma, and adult T-cell leukemia/lymphoma.

Aspect 51. A method for treating a cancer in a subject suffering therefrom, wherein the cancer is characterized by a reduction or absence of methylthioadenosine phosphorylase (MTAP) gene expression, the absence of the MTAP gene, or reduced function of MTAP protein, as compared to cancers where the MTAP gene or protein is present and/or fully functioning, the method comprising administering to the subject a therapeutically effective amount of a compound, or a pharmaceutically acceptable salt thereof, according to any one of Aspects 1 to 36.

Aspect 52. A compound according to any one of Aspects 1 to 36, or a pharmaceutically acceptable salt thereof, for inhibiting the synthesis of S-adenosyl methionine (SAM).

Aspect 53. A compound according to any one of Aspects 1 to 36, or a pharmaceutically acceptable salt thereof, for treating a cancer in a subject suffering therefrom.

Aspect 54. The compound according to Aspect 53, wherein the cancer is an MTAP-deleted cancer.

Aspect 55. The compound according to Aspect 53 or 54, wherein the cancer is selected from the group consisting of mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

Aspect 56. The compound according to Aspect 53 or 54, wherein the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, and breast cancer.

Aspect 57. The compound according to Aspect 56, wherein the cancer is a lung cancer is selected from the group consisting of non-small cell lung cancer, small cell lung cancer, adenocarcinoma of the lung, and squamous cell carcinoma of the lung.

Aspect 58. The compound according to Aspect 56, wherein the cancer is triple negative breast cancer (TNBC).

Aspect 59. The compound according to Aspect 56, wherein the cancer is a brain tumor selected from the group consisting of glioma, glioblastoma, astrocytoma, meningioma, medulloblastoma, peripheral neuroectodermal tumors, and craniopharyngioma.

Aspect 60. The compound according to Aspect 56, wherein the cancer is a lymphoma selected from the group consisting of mantle cell lymphoma, Hodgkin lymphoma, non-Hodgkin lymphoma, Burkitt lymphoma, diffuse large B-cell lymphoma (DLBCL), and adult T-cell leukemia/lymphoma.

### EXAMPLES

The present disclosure will be more fully understood by reference to the following examples. The examples should not, however, be construed as limiting the scope of the present disclosure.

### Units and Terms List

| | |
|---|---|
| anhy. | anhydrous |
| aq. | aqueous |
| min | minute(s) |
| mL | milliliter |
| mmol | millimole(s) |
| mol | mole(s) |
| MS | mass spectrometry |
| NMR | nuclear magnetic resonance |
| TLC | thin layer chromatography |
| HPLC | high-performance liquid chromatography |
| RT (r.t.) | room temperature |

NMR Spectra

| | |
|---|---|
| Hz | hertz |
| δ | chemical shift |
| J | coupling constant |
| s | singlet |
| d | doublet |
| t | triplet |
| q | quartet |
| m | multiplet |
| br | broad |
| qd | quartet of doublets |
| dquin | doublet of quintets |
| dd | doublet of doublets |
| dt | doublet of triplets |

Solvents and Reagents:

| | |
|---|---|
| CHCl₃ | chloroform |
| DCM | dichloromethane |
| DMF | dimethylformamide |
| Et₂O | diethyl ether |
| EtOH | ethyl alcohol |
| EtOAc | ethyl acetate |
| EA | ethyl acetate |
| MeOH | methyl alcohol |
| MeCN | acetonitrile |
| PE | petroleum ether |
| THF | tetrahydrofuran |
| AcOH | acetic acid |
| HCl | hydrochloric acid |
| H₂SO₄ | sulfuric acid |
| NH₄Cl | ammonium chloride |
| KOH | potassium hydroxide |
| NaOH | sodium hydroxide |
| K₂CO₃ | potassium carbonate |
| Na₂CO₃ | sodium carbonate |
| TFA | trifluoroacetic acid |
| Na₂SO₄ | sodium sulfate |
| NaBH₄ | sodium borohydride |
| NaHCO₃ | sodium bicarbonate |
| LiHMDS | lithium hexamethyldisilylamide |
| NaHMDS | sodium hexamethyldisilylamide |
| LAH | lithium aluminum hydride |
| NaBH₄ | sodium borohydride |
| LDA | lithium diisopropylamide |
| Et₃N | triethylamine |
| DMAP | 4-(dimethylamino)pyridine |
| DIPEA | N,N-diisopropylethylamine |
| NH₄OH | ammonium hydroxide |
| EDCI | 1-ethyl-3-(3-dimethylaminopropyl)carbodiimide |
| HOBt | 1-hydroxybenzotriazole |
| HATU | O-(7-azabenzotriazol-1-yl)-N,N,N′,N′-tetra-methyluronium |
| Xphos | 2-Dicyclohexylphosphino-2′,4′,6′-triisopropylbiphenyl |
| BINAP | 2,2′-bis(diphenylphosphanyl)-1,1′-binaphthyl |

### GENERAL EXPERIMENTAL

In the following examples, the reagents and solvents were purchased from commercial sources (such as Alfa, Acros, Sigma Aldrich, TCI and Shanghai Chemical Reagent Company), and used without further purification unless otherwise specified. Flash chromatography was performed on an Ez Purifier III using column with silica gel particles of 200-300 mesh. Analytical and preparative thin layer chromatography (TLC) plates were HSGF 254 (0.15-0.2 mm thickness, Shanghai Anbang Company, China). Nuclear magnetic resonance (NMR) spectra were obtained on a Brucker AMX-400 NMR (Brucker, Switzerland). Chemical shifts were reported in parts per million (ppm, 6) downfield from tetramethylsilane. Mass spectra were given with electrospray ionization (ESI) from a Waters LCT TOF Mass Spectrometer (Waters, USA). HPLC chromatographs were record on an Agilent 1200 Liquid Chromatography (Agilent, USA, column: Ultimate 4.6 mm×50 mm, 5 μm, mobile phase A: 0.1% formic acid in water; mobile phase B: acetonitrile). Microwave reactions were run on an Initiator 2.5 Microwave Synthesizer (Biotage, Sweden).

### General Procedure I

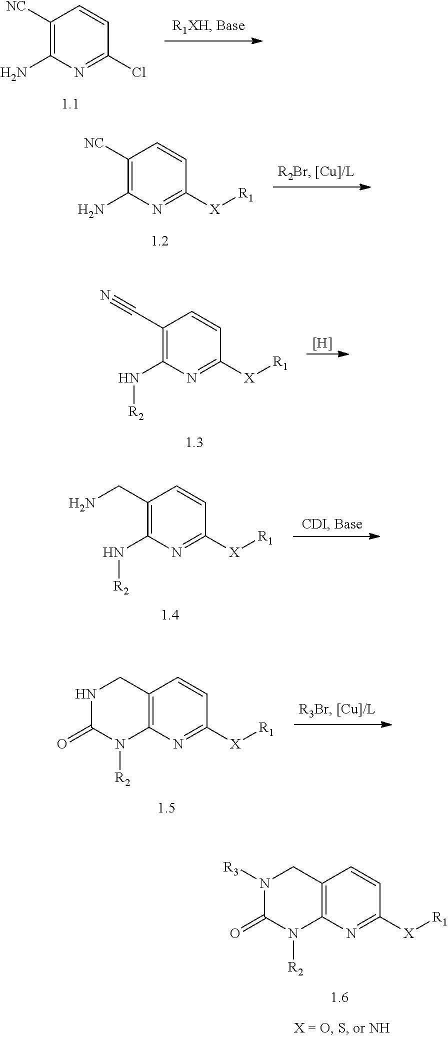

Compounds of structure 1.6 were obtained through the scheme depicted as General Procedure I. Beginning with nitrile 1.1, base mediated aromatic substitution was used to introduce the desired Ri group in structure 1.2. A copper mediated N—C cross-coupling reaction was then used to introduce the desired R₂ group in structure 1.3. Nitrile 1.3 was then reduced under hydrogenation conditions to afford diamine 1.4. Diamine 1.4 was converted to cyclic urea 1.5 using CDI. Lastly, the desired R₃ group was introduced using a copper mediated N—C cross-coupling to afford compounds of structure 1.6.

### Preparation of Example 101 Via General Procedure I

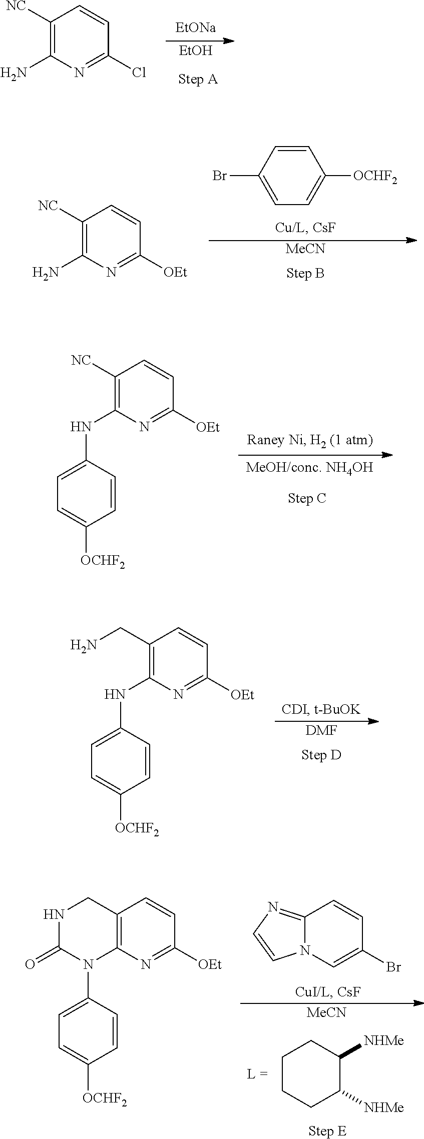

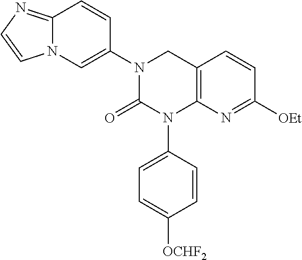 [SMILES: CCOc1ccc2c(n1)N(c1ccc(OC(F)F)cc1)C(=O)N(c1ccc3nccn3c1)C2]

### Step A: 2-amino-6-ethoxynicotinonitrile

To a solution of 2-amino-6-chloronicotinonitrile (5.0 g, 32.6 mmol, 1.0 eq.) in EtOH (30 mL) was added EtONa (6.7 g, 97.8 mmol, 3.0 eq.) in portions, then the reaction mixture was stirred at room temperature for 30 min. The progress of the reaction was monitored by LC-MS (ESI), after completion, the reaction was quenched with ice water (50 mL), the resulting precipitate was filtered, the filter cake was collected and dried under reduced pressure, to afford 2-amino-6-ethoxynicotinonitrile (3.9 g, 73% yield) as a yellow solid. LC-MS (ESI): m/z 252 [M+H]⁺.

### Step B: 2-((4-(difluoromethoxy)phenyl)amino)-6-ethoxynicotinonitrile

A mixture of 2-amino-6-ethoxynicotinonitrile (3.9 g, 23.9 mmol, 1.0 eq.), CuI (4.4 g, 23.9 mmol, 1.0 eq.), CsF (10.7 g, 71.7 mmol, 3.0 eq.), 1-bromo-4-(difluoromethoxy)benzene (7.8 g, 35.1 mmol, 1.5 eq.) and N¹,N²-dimethylcyclohexane-1,2-diamine (6.8 g, 47.8 mmol, 2.0 eq.) in MeCN (50 ml) was stirred 100° C. under N₂ atmosphere for 15 hrs. The reaction mixture was diluted with H₂O (100 ml), extracted with EtOAc (100 mL×3), the combined organic layers were washed with brine (50 ml), dried over with Na₂SO₄, concentrated under reduced pressure, the residue was purified by flash column chromatography on silica gel to afford 2-((4-(difluoromethoxy)phenyl)amino)-6-ethoxynicotinonitrile (3.7 g, 51% yield) as a white solid. LC-MS (ESI): m/z 306 [M+H]⁺.

### Step C: 2-((4-(difluoromethoxy)phenyl)amino)-6-ethoxynicotinonitrile

To a solution of 2-((4-(difluoromethoxy)phenyl)amino)-6-ethoxynicotinonitrile (1.0 g, 3.2 mmol, 1.0 eq.) in MeOH (40 mL) was added Raney Ni (300 mg) and conc. NH₄OH (4 mL), the reaction mixture stirred under H₂ balloon (l atm) at room temperature for 15 hrs. The progress of the reaction was monitored by LC-MS, after completion, the catalyst was filtered through a short pad of Celite®, the filtrate was concentrated under reduced pressure to afford crude 3-(aminomethyl)-N-(4-(difluoromethoxy)phenyl)-6-ethoxypyridin-2-amine (1.0 g) as a pale yellow oil, which was used in next step without further purification. LC-MS (ESI): m/z 310 [M+H]⁺.

### Step D: 1-(4-(difluoromethoxy)phenyl)-7-ethoxy-3,4-dihydropyrido[2,3-d]pyrimidin-2(1H)-one

To a solution of 3-(aminomethyl)-N-(4-(difluoromethoxy)phenyl)-6-ethoxypyridin-2-amine (1.0 g, 3.2 mmol, 1.0 eq.) in anhy. DMF (20 mL) was added CDI (1.1 g, 6.4 mmol, 2.0 eq.) and t-BuOK (1.45 g, 12.8 mmol, 4.0 eq.) in one portion, the resulting mixture was stirred at 60° C. under N₂ atmosphere for 4 hrs. The progress of the reaction was monitored by LC-MS, after completion, the reaction was quenched with ice water (50 mL), extracted with DCM (40 mL×3), the combined organic layers were dried over with Na₂SO₄, concentrated under reduced pressure and purified by flash column chromatography on silica gel to afford 1-(4-(difluoromethoxy)phenyl)-7-ethoxy-3,4-dihydropyrido[2,3-d]pyrimidin-2(1H)-one (0.9 g, 83% yield) as a white solid. LC-MS (ESI): m/z 336 [M+H]⁺.

### Step E: 1-(4-(difluoromethoxy)phenyl)-7-ethoxy-3-(imidazo[1,2-a]pyridin-6-yl)-3,4-dihydropyrido[2,3-d]pyrimidin-2(1H)-one

A mixture of 1-(4-(difluoromethoxy)phenyl)-7-ethoxy-3,4-dihydropyrido[2,3-d]pyrimidin-2(1H)-one (50 mg, 0.15 mmol, 1.0 eq.), 6-bromoimidazo[1,2-a]pyridine (44 mg, 0.22 mmol, 1.5 eq.), CsF (45 mg, 0.3 mmol, 2.0 eq.), CuI (28 mg, 0.15 mmol, 1.0 eq.) and N¹,N²-dimethylcyclohexane-1,2-diamine (42 mg, 0.3 mmol, 2.0 eq.) in MeCN (3 mL) was stirred at 60° C. under N₂ atmosphere for 15 hrs. The reaction mixture was diluted with EtOAc (40 mL), washed with H₂O (2×10 mL), dried over Na₂SO₄, concentrated under reduced pressure and purified by RP-prep-HPLC to afford 1-(4-(difluoromethoxy)phenyl)-7-ethoxy-3-(imidazo[1,2-a]pyridin-6-yl)-3,4-dihydropyrido[2,3-d]pyrimidin-2(1H)-one (Example 101).

¹H NMR (400 MHz, DMSO-d₆) δ (ppm): 8.75 (s, 1H), 7.96 (s, 1H), 7.72-7.50 (m, 3H), 7.46-7.33 (m, 3H), 7.30 (t, J_{HF}=76 Hz, 1H), 7.28-7.13 (m, 2H), 6.44 (d, J=8.0 Hz, 1H), 4.92 (s, 2H), 3.86 (q, J=8.0 Hz, 2H), 1.05 (7, J=8.0 Hz, 3H).

LC-MS (ESI): m/z 452 [M+H]⁺.

The procedure set forth above for General Procedure I was used to synthesize the following compounds by using appropriate starting materials:

| Cpd No. | Structure | Characterization |
|---|---|---|
| Example 102 | 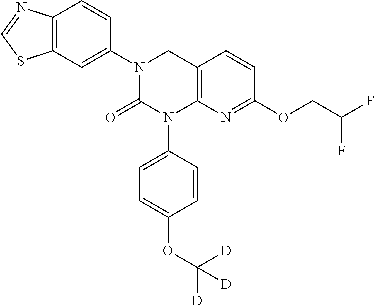 [SMILES: [2H]C([2H])([2H])Oc1ccc(N2C(=O)N(c3ccc4ncsc4c3)Cc3ccc(OCC(F)F)nc32)cc1] | LC-MS (ESI): m/z 472.0 [M + H]+. 1H NMR (400 MHz, DMSO-d₆) δ: 9.39 (s, 1H), 8.24 (d, J = 2.4 Hz, 1H), 8.10 (d, J = 8.8 Hz, 1H), 7.67 (d, J = 8.0 Hz, 1H), 7.63 (dd, J = 8.8 Hz, 2.4 Hz, 1H), 7.28 (d, J = 8.8 Hz, 2H), 7.00 (d, J = 8.8 Hz, 2H), 6.55 (d, J = 8.4 Hz, 1H), 6.02 (tt, J_{HF} = 55.6 Hz, J = 4.0 Hz, 1H), 5.00 (s, 2H), 4.07 (td, J_{HF} = 14.8 Hz, J = 4.0 Hz, 2H). |
| | 3-(benzo[d]thiazol-6-yl)-7- | |
| | (2,2-difluoroethoxy)-1-(4- | |
| | (methoxy-d3)phenyl)-3,4- | |
| | dihydropyrido[2,3- | |
| | d]pyrimidin-2(1H)-one | |
| Example 103 | 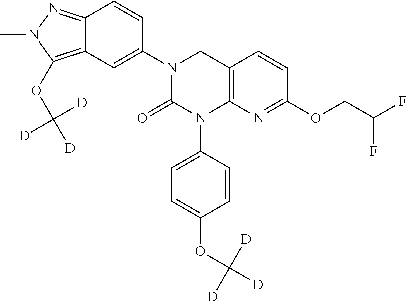 [SMILES: [2H]C([2H])([2H])Oc1ccc(N2C(=O)N(c3ccc4nn(C)c(OC([2H])([2H])[2H])c4c3)Cc3ccc(OCC(F)F)nc32)cc1] | LC-MS (ESI): m/z 502.1 [M + H]+. 1H NMR (400 MHz, DMSO-d₆) δ: 7.81 (d, J = 1.4 Hz, 1H), 7.66 (d, J = 8.1 Hz, 1H), 7.37 (d, J = 9.2 Hz, 1H), 7.25 (d, J = 8.8 Hz, 2H), 7.18 (dd, J = 9.2 Hz, 1.9 Hz, 1H), 7.00 (d, J = 8.8 Hz, 2H), 6.53 (d J = 8.0 Hz, 1H), 6.02 (tt, J_{HF} = 55.4 Hz, J = 4.0 Hz, 1H), 4.93 (s, 2H), 4.08 (td, J_{HF} = 14.5 Hz, J = 4.0 Hz, 2H), 3.87 (s, 3H). |
| | 7-(2,2-difluoroethoxy)-3-(3- | |
| | (methoxy-d3)-2-methyl-2H- | |
| | indazol-5-yl)-1-(4- | |
| | (methoxy-d3)phenyl)-3,4- | |
| | dihydropyrido[2,3- | |
| | d]pyrimidin-2(1H)-one | |
| Example 104 | 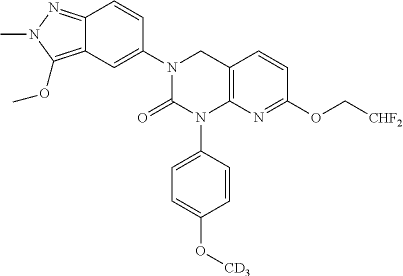 [SMILES: [2H]C([2H])([2H])Oc1ccc(N2C(=O)N(c3ccc4nn(C)c(OC)c4c3)Cc3ccc(OCC(F)F)nc32)cc1] | LC-MS (ESI): m/z 499.1 [M + H]+. 1H NMR (400 MHz, DMSO-d₆) δ: 7.81 (d, J = 1.3 Hz, 1H), 7.66 (d, J = 8.1 Hz, 1H), 7.37 (d, J = 9.3 Hz, 1H), 7.25 (d, J = 8.9 Hz, 2H), 7.18 (dd, J = 9.3 Hz, 1.9 Hz, 1H), 6.99 (d, J = 8.9 Hz, 2H), 6.53 (d, J = 8.0 Hz, 1H), 6.02 (tt, J_{HF} = 55.4 Hz, J = 3.8 Hz, 1H), 4.92 (s, 2H), 4.28 (s, 3H), 4.07 (td, J_{HF} = 14.4 Hz, J = 3.9 Hz, 2H), 3.87 (s, 3H). |
| | 7-(2,2-difluoroethoxy)-3-(3- | |
| | methoxy-2-methyl-2H- | |
| | indazol-5-yl)-1-(4- | |
| | (methoxy-d3)phenyl)-3,4- | |
| | dihydropyrido[2,3- | |
| | d]pyrimidin-2(1H)-one | |
| Example 105 | 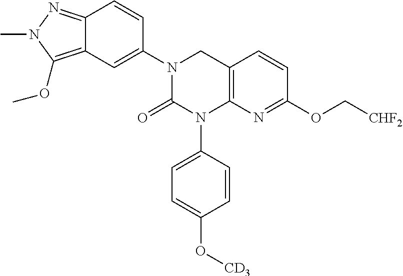 | LC-MS (ESI): m/z 483.1 [M + H]+. 1H NMR (400 MHz, DMSO-d₆) δ: 7.67 (s, 1H), 7.65 (s, 1H), 7.48 (d, J = 9.1 Hz, 1H), 7.29-7.19 (m, 3H), 7.00 (d, J = 8.8 Hz, 2H), 6.53 (d, J = 8.0 Hz, 1H), 6.02 (tt, J_{HF} = 55.5 Hz, J = 3.9 Hz, 1H), 4.92 (s, 2H), 4.07 (td, J_{HF} = 14.5 Hz, J = 3.9 Hz, 2H), 4.05 (s, 3H), 2.60 (s, 3H). |
| | 7-(2,2-difluoroethoxy)-3- | |
| | (2,3-dimethyl-2H-indazol-5- | |
| | yl)-1-(4-(methoxy- | |
| | d3)phenyl)-3,4- | |
| | dihydropyrido[2,3- | |
| | d]pyrimidin-2(1H)-one | |
| Example 106 | 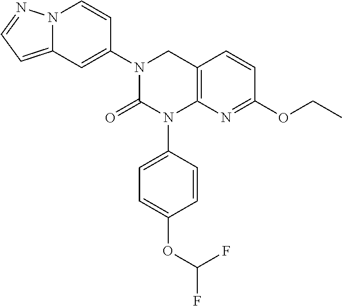 [SMILES: CCOc1ccc2c(n1)N(c1ccc(OC(F)F)cc1)C(=O)N(c1ccn3nccc3c1)C2] | LC-MS (ESI): m/z 452.0 [M + H]+. 1H NMR (400 MHz, DMSO-d₆) δ: 8.62 (d, J = 7.6 Hz, 1H), 7.98 (d, J = 2.0 Hz, 1H), 7.66 (d, J = 2.4 Hz, 1H), 7.63 (d, J = 8.0 Hz, 1H), 7.43 (d, J = 8.8 Hz, 2H), 7.31 (t, J_{HF} = 74.0 Hz, 1H), 7.26 (d, J = 8.8 Hz, 2H), 7.02 (dd, J = 7.2 Hz, 2.4 Hz, 1H), 6.59 (d, J = 2.0 Hz, 1H), 6.45 (d, J = 8.0 Hz, 1H), 4.98 (s, 2H), 3.86 (q, J = 7.2 Hz, 2H), 1.06 (t, J = 7.2 Hz, 3H). |
| | 1-(4- | |
| | (difluoromethoxy)phenyl)- | |
| | 7-ethoxy-3-(pyrazolo[1,5- | |
| | a]pyridin-5-yl)-3,4- | |
| | dihydropyrido[2,3- | |
| | d]pyrimidin-2(1H)-one | |
| Example 107 | 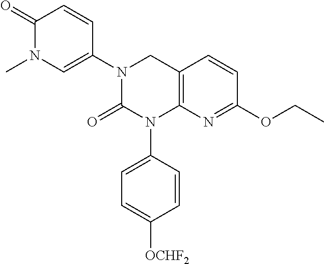 [SMILES: CCOc1ccc2c(n1)N(c1ccc(OC(F)F)cc1)C(=O)N(c1ccc(=O)n(C)c1)C2] | LC-MS (ESI): m/z 443.0 [M + H]+. 1H NMR (400 MHz, DMSO-d₆) δ: 7.91 (s, 1H), 7.56 (t, J = 8.0 Hz, 2H), 7.30 (t, J_{HF} = 74.4 Hz, 1H), 7.36-7.23 (m, 4H), 6.41 (t, J = 8.0 Hz, 2H), 4.77 (s, 2H), 3.85 (q, J = 6.4 Hz, 2H), 3.43 (s, 3H), 1.04 (t, J = 6.4 Hz, 3H). |
| | 1-(4- | |
| | (difluoromethoxy)phenyl)- | |
| | 7-ethoxy-3-(1-methyl-6- | |
| | oxo-1,6-dihydropyridin-3- | |
| | yl)-3,4-dihydropyrido[2,3- | |
| | d]pyrimidin-2(1H)-one | |
| Example 108 | 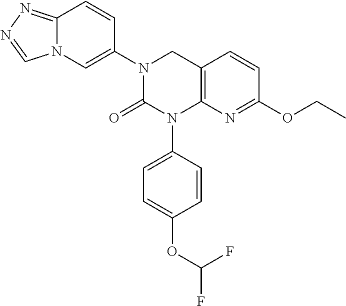 [SMILES: CCOc1ccc2c(n1)N(c1ccc(OC(F)F)cc1)C(=O)N(c1ccc3nncn3c1)C2] | LC-MS (ESI): m/z 453.0 [M + H]+. 1H NMR (400 MHz, DMSO-d₆) δ: 9.27 (s, 1H), 8.76 (s, 1H), 7.79 (d, J = 8.8 Hz, 1H), 7.61 (d, J = 8.0 Hz, 1H), 7.54 (d, J = 9.6 Hz, 1H), 7.42 (d, J = 8.8 Hz, 2H), 7.30 (t, J_{HF} = 74.0 Hz, 1H), 7.26 (d, J = 8.8 Hz, 2H), 6.46 (d, J = 8.0 Hz, 1H), 4.94 (s, 2H), 3.87 (q, J = 7.2 Hz, 2H), 1.05 (t J = 7.2 Hz, 3H). |
| | 3-([1,2,4]triazolo[4,3- | |
| | a]pyridin-6-yl)-1-(4- | |
| | (difluoromethoxy)phenyl)- | |
| | 7-ethoxy-3,4- | |
| | dihydropyrido[2,3- | |
| | d]pyrimidin-2(1H)-one | |
| Example 109 | 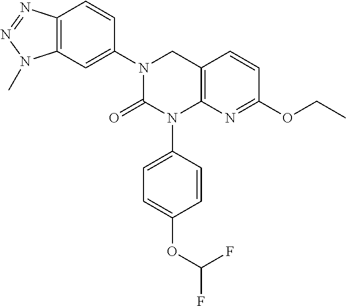 [SMILES: CCOc1ccc2c(n1)N(c1ccc(OC(F)F)cc1)C(=O)N(c1ccc3nnn(C)c3c1)C2] | LC-MS (ESI): m/z 467.0 [M + H]+. 1H NMR (400 MHz, DMSO-d₆) δ: 8.02 (d, J = 9.2 Hz, 1H), 7.93 (d, J = 1.6 Hz, 1H), 7.63 (d, J = 8.4 Hz, 1H), 7.50 (d, J = 8.0 Hz, 1H), 7.42 (d, J = 8.8 Hz, 2H), 7.31 (t, J_{HF} = 74.0 Hz, 1H), 7.26 (d, J = 8.8 Hz, 2H), 6.45 (d, J = 8.0 Hz, 1H), 5.01 (s, 2H), 4.30 (s, 3H), 3.87 (q, J = 7.2 Hz, 2H), 1.06 (t, J = 7.2 Hz, 3H). |
| | 1-(4- | |
| | (difluoromethoxy)phenyl)- | |
| | 7-ethoxy-3-(pyrazolo[1,5- | |
| | a]pyridin-5-yl)-3,4- | |
| | dihydropyrido[2,3- | |
| | d]pyrimidin-2(1H)-one | |
| Example 110 | 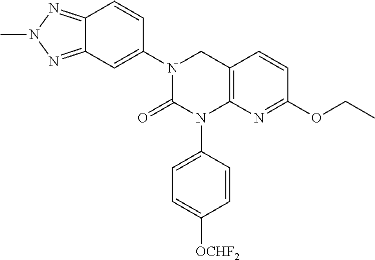 [SMILES: CCOc1ccc2c(n1)N(c1ccc(OC(F)F)cc1)C(=O)N(c1ccc3nn(C)nc3c1)C2] | LC-MS (ESI): m/z 467.0 [M + H]+. 1H NMR (400 MHz, DMSO-d₆) δ: 7.92 (s, 1H), 7.88 (d, J = 9.2 Hz, 1H), 7.62 (d, J = 8.0 Hz, 1H), 7.52 (d, J = 8.8 Hz, 1H), 7.42 (d, J = 8.4 Hz, 2H), 7.31 (t, J_{HF} = 74.0 Hz, 1H), 7.25 (d, J = 8.4 Hz, 2H), 6.43 (d, J = 8.0 Hz, 1H), 5.00 (s, 2H), 4.50 (s, 3H), 3.86 (q, J = 7.2 Hz, 2H), 1.06 (t, J = 7.2 Hz, 3H). |
| | 1-(4- | |
| | (difluoromethoxy)phenyl)- | |
| | 7-ethoxy-3-(2-methyl-2H- | |
| | benzo[d][1,2,3]triazol-5-yl)- | |
| | 3,4-dihydropyrido[2,3- | |
| | d]pyrimidin-2(1H)-one | |
| Example 111 | 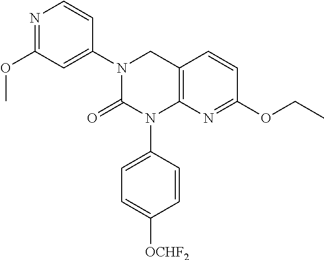 [SMILES: CCOc1ccc2c(n1)N(c1ccc(OC(F)F)cc1)C(=O)N(c1ccnc(OC)c1)C2] | LC-MS (ESI): m/z 443.0 [M + H]+. 1H NMR (400 MHz, DMSO-d₆) δ: 8.10 (d, J = 5.6 Hz, 1H), 7.63 (d, J = 8.0 Hz, 1H), 7.43 (d, J = 8.8 Hz, 2H), 7.32 (t, J_{HF} = 74.0 Hz, 1H), 7.26 (d, J = 8.8 Hz, 2H), 7.12 (d, J = 5.6 Hz, 1H), 6.83 (d, J = 1.6 Hz, 1H), 6.45 (d, J = 8.0 Hz, 1H), 4.95 (s, 2H), 3.86 (q, J = 7.2 Hz, 2H), 3.85 (s, 3H), 1.05 (t, J = 7.2 Hz, 3H). |
| | 1-(4- | |
| | (difluoromethoxy)phenyl)- | |
| | 7-ethoxy-3-(2- | |
| | methoxypyridin-4-yl)-3,4- | |
| | dihydropyrido[2,3- | |
| | d]pyrimidin-2(1H)-one | |
| Example 112 | 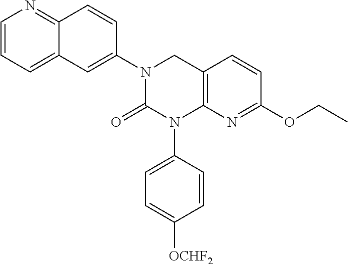 [SMILES: CCOc1ccc2c(n1)N(c1ccc(OC(F)F)cc1)C(=O)N(c1ccc3ncccc3c1)C2] | LC-MS (ESI): m/z 463.2 [M + H]+. 1H NMR (400 MHz, DMSO-d₆) δ: 8.89 (d, J = 2.4 Hz, 1H), 8.37 (d, J = 8.0 Hz, 1H), 8.02 (s, 1H), 8.00 (s, 1H), 7.88 (dd, J = 9.2 Hz, 2.4 Hz, 1H), 7.65 (d, J = 8.0 Hz, 1H), 7.56 (dd, J = 8.4 Hz, 4.0 Hz, 1H), 7.45 (d, J = 8.8 Hz, 2H), 7.31 (t, J_{HF} = 74.0 Hz, 1H), 7.27 (d, J = 8.8 Hz, 2H), 6.46 (d, J = 8.0 Hz, 1H), 5.07 (s, 2H), 3.88 (q, J = 7.2 Hz, 2H), 1.07 (t, J = 7.2 Hz, 3H). |
| | 1-(4- | |
| | (difluoromethoxy)phenyl)- | |
| | 7-ethoxy-3-(quinolin-6-yl)- | |
| | 3,4-dihydropyrido[2,3- | |
| | d]pyrimidin-2(1H)-one | |
| Example 113 | 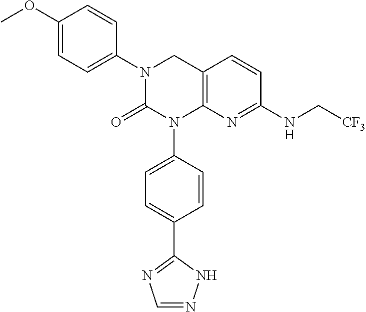 [SMILES: COc1ccc(N2Cc3ccc(NCC(F)(F)F)nc3N(c3ccc(-c4ncn[nH]4)cc3)C2=O)cc1] | LC-MS (ESI): m/z 496.0 [M + H]+. 1H NMR (400 MHz, DMSO-d₆) δ: 14.34 (s, 1H), 8.43 (s, 1H), 8.05 (d, J = 8.4 Hz, 2H), 7.38-7.33 (m, 5H), 7.07 (t, J = 6.8 Hz, 1H), 6.96 (d, J = 8.8 Hz, 2H), 6.25 (d, J = 8.0 Hz, 1H), 4.76 (s, 2H), 3.77 (s, 3H), 3.73-3.62 (m, 2H). |
| | 1-(4-(1H-1,2,4-triazol-5- | |
| | yl)phenyl)-3-(4- | |
| | methoxyphenyl)-7-((2,2,2- | |
| | trifluoroethyl)amino)-3,4- | |
| | dihydropyrido[2,3- | |
| | d]pyrimidin-2(1H)-one | |
| | (Synthesized using 3-(4- | |
| | bromophenyl)-1-((2- | |
| | (trimethylsilyl)ethoxy)meth- | |
| | yl)-1H-1,2,4-triazole (Ref: | |
| | WO2008156726 A1) and de- | |
| | protection with TFA via | |
| | general procedure IV (Step | |
| | F)) | |
| Example 114 | 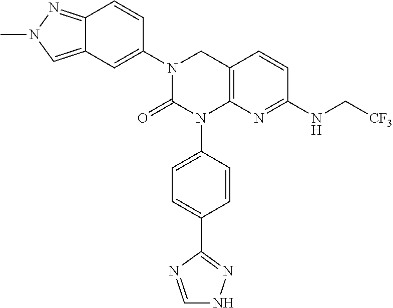 [SMILES: Cn1cc2cc(N3Cc4ccc(NCC(F)(F)F)nc4N(c4ccc(-c5nc[nH]n5)cc4)C3=O)ccc2n1] | LC-MS (ESI): m/z 520.2 [M + H]+. 1H NMR (400 MHz, DMSO-d₆) δ: 14.43 (s, 1H), 8.72-8.23 (m, 3H), 8.06 (d, J = 8.0 Hz, 2H), 7.70 (s, 1H), 7.58 (d, J = 9.2 Hz, 1H), 7.40 (d, J = 8.0 Hz, 2H), 7.29 (d, J = 8.8 Hz, 1H), 7.09 (s, 1H), 6.26 (d, J = 8.0 Hz, 1H), 4.85 (s, 2H), 4.17 (s, 3H), 3.75- 3.66 (m, 2H). |
| | 1-(4-(1H-1,2,4-triazol-3- | |
| | yl)phenyl)-3-(2-methyl-2H- | |
| | indazol-5-yl)-7-((2,2,2- | |
| | trifluoroethyl)amino)-3,4- | |
| | dihydropyrido[2,3- | |
| | d]pyrimidin-2(1H)-one | |
| | (Synthesized using 3-(4- | |
| | bromophenyl)-1-((2- | |
| | (trimethylsilyl)ethoxy)meth- | |
| | yl)-1H-1,2,4-triazole (Ref: | |
| | WO2008156726 A1) and | |
| | de-protection with TFA via | |
| | general procedure IV (Step | |
| | F)) | |
| Example 115 | 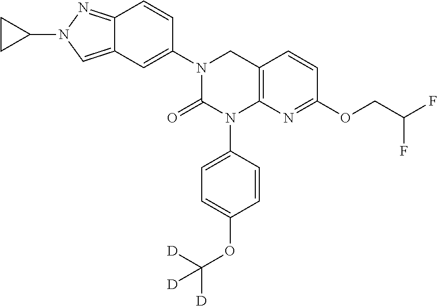 [SMILES: [2H]C([2H])([2H])Oc1ccc(N2C(=O)N(c3ccc4nn(C5CC5)cc4c3)Cc3ccc(OCC(F)F)nc32)cc1] | LC-MS: m/z 495.2 (M + H)+. 1H NMR (400 MHz, DMSO-d₆) δ: 8.47 (s, 1H), 7.68-7.63 (m, 2H), 7.55 (d, J = 9.2 Hz, 1H), 7.28-7.23 (m, 3H), 6.99 (d, J = 8.8 Hz, 2H), 6.53 (d, J = 8.0 Hz, 1H), 6.01 (tt, J_{HF} = 55.2 Hz, 4.0 Hz, 1H), 4.92 (s, 2H), 4.15 (hept, J = 4.0 Hz, 1H), 4.07 (td, J_{HF} = 14.4 Hz, 4.0 Hz, 2H), 1.31-1.24 (m, 2H), 1.15- 1.05 (m, 2H). |
| | 3-(2-cyclopropyl-2H- | |
| | indazol-5-yl)-7-(2,2- | |
| | difluoroethoxy)-1-(4- | |
| | methoxy-d3-phenyl)-3,4- | |
| | dihydropyrido[2,3- | |
| | d]pyrimidin-2(1H)-one | |
| Example 116 | 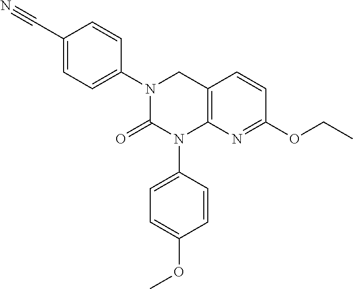 [SMILES: CCOc1ccc2c(n1)N(c1ccc(OC)cc1)C(=O)N(c1ccc(C#N)cc1)C2] | LC-MS: m/z 401.2 (M + H)+. 1H NMR (400 MHz, DMSO-d₆) δ: 7.86 (d, J = 8.8 Hz, 2H), 7.64-7.58 (m, 3H), 7.26 (d, J = 8.8 Hz, 2H), 6.99 (d, J = 9.2 Hz, 2H), 6.43 (d, J = 8.0 Hz, 1H), 4.95 (s, 2H), 3.87 (q, J = 7.2 Hz, 2H), 3.80 (s, 3H), 1.06 (t, J = 7.2 Hz, 3H). |
| | 4-(7-ethoxy-1-(4- | |
| | methoxyphenyl)-2-oxo-1,2- | |
| | dihydropyrido[2,3- | |
| | d]pyrimidin-3(4H)- | |
| | yl)benzonitrile | |
| Example 117 | 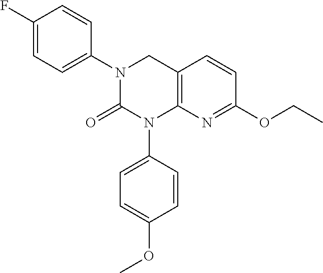 [SMILES: CCOc1ccc2c(n1)N(c1ccc(OC)cc1)C(=O)N(c1ccc(F)cc1)C2] | LC-MS: m/z 394.1 (M + H)+. 1H NMR (400 MHz, DMSO-d₆) δ: 7.57 (d, J = 8.4 Hz, 1H), 7.49-7.42 (m, 2H), 7.27- 7.19 (m, 4H), 6.98 (d, J = 8.8 Hz, 2H), 6.40 (d, J = 8.4 Hz, 1H), 4.85 (s, 2H), 3.86 (q, J = 7.2 Hz, 2H), 3.79 (s, 3H), 1.06 (t, J = 7.2 Hz, 3H). |
| | 7-ethoxy-3-(4- | |
| | fluorophenyl)-1-(4- | |
| | methoxyphenyl)-3,4- | |
| | dihydropyrido[2,3- | |
| | d]pyrimidin-2(1H)-one | |
| Example 118 | 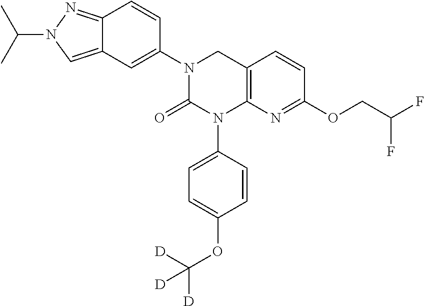 [SMILES: [2H]C([2H])([2H])Oc1ccc(N2C(=O)N(c3ccc4nn(C(C)C)cc4c3)Cc3ccc(OCC(F)F)nc32)cc1] | LC-MS: m/z 497.2 (M + H)+. 1H NMR (400 MHz, DMSO-d₆) δ: 8.41 (s, 1H), 7.68 (d, J = 1.6 Hz, 1H), 7.65 (d, J = 8.4 Hz, 1H), 7.60 (d, J = 8.8 Hz, 1H), 7.28- 7.24 (m, 3H), 6.99 (d, J = 8.8 Hz, 2H), 6.52 (d, J = 8.0 Hz, 1H), 6.01 (tt, J_{HF} = 55.6 Hz, 4.0 Hz, 1H), 4.92 (s, 2H), 4.82 (hept, J = 6.8 Hz, 1H), 4.07 (td, J_{HF} = 14.4 Hz, 4.0 Hz, 2H), 1.55 (d, J = 6.8 Hz, 6H). |
| | 7-(2,2-difluoroethoxy)-3-(2- | |
| | isopropyl-2H-indazol-5-yl)- | |
| | 1-(4-methoxy-d3-phenyl)- | |
| | 3,4-dihydropyrido[2,3- | |
| | d]pyrimidin-2(1H)-one | |
| Example 119 | 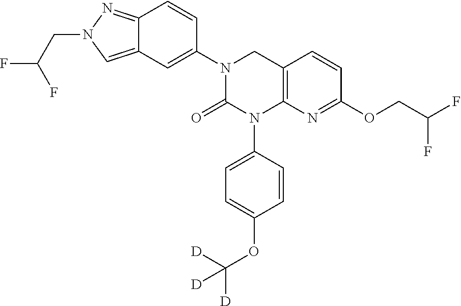 [SMILES: [2H]C([2H])([2H])Oc1ccc(N2C(=O)N(c3ccc4nn(CC(F)F)cc4c3)Cc3ccc(OCC(F)F)nc32)cc1] | LC-MS: m/z 519.2 (M + H)+. 1H NMR (400 MHz, DMSO-d₆) δ: 8.46 (s, 1H), 7.74 (d, J = 1.2 Hz, 1H), 7.66 (d, J = 8.4 Hz, 1H), 7.62 (d, J = 9.2 Hz, 1H), 7.32 (dd, J = 9.2 Hz, 2.0 Hz, 1H), 7.26 (d, J = 8.8 Hz, 2H), 7.00 (d, J = 8.8 Hz, 2H), 6.53 (d, J = 8.0 Hz, 1H), 6.51 (tt, J_{HF} = 55.2 Hz, 4.0 Hz, 1H), 6.01 (tt, J_{HF} = 55.6 Hz, 4.0 Hz, 1H), 4.97 (td, J_{HF} = 14.4 Hz, 4.0 Hz, 2H), 4.94 (s, 2H), 4.07 (td, J_{HF} = 14.4 Hz, 4.0 Hz, 2H). |
| | 7-(2,2-difluoroethoxy)-3-(2- | |
| | (2,2-difluoroethyl)-2H- | |
| | indazol-5-yl)-1-(4-methoxy- | |
| | d3-phenyl)-3,4- | |
| | dihydropyrido[2,3- | |
| | d]pyrimidin-2(1H)-one | |
| Example 120 | 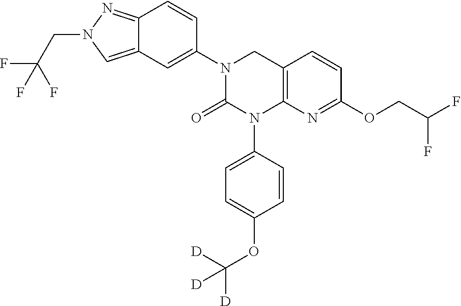 [SMILES: [2H]C([2H])([2H])Oc1ccc(N2C(=O)N(c3ccc4nn(CC(F)(F)F)cc4c3)Cc3ccc(OCC(F)F)nc32)cc1] | LC-MS: m/z 537.2 (M + H)+. 1H NMR (400 MHz, DMSO-d₆) δ: 8.52 (s, 1H), 7.76 (s, 1H), 7.68-7.61 (m, 2H), 7.36 (d, J = 8.8 Hz, 1H), 7.27 (d, J = 8.8 Hz, 2H), 7.00 (d, J = 8.8 Hz, 2H), 6.53 (d, J = 8.0 Hz, 1H), 6.01 (tt, J_{HF} = 55.6 Hz, 4.0 Hz, 1H), 5.50 (q, J = 4.8 Hz, 2H), 4.94 (s, 2H), 4.07 (td, J_{HF} = 14.4 Hz, 4.0 Hz, 2H). |
| | 7-(2,2-difluoroethoxy)-1-(4- | |
| | methoxy-d3-phenyl)-3-(2- | |
| | (2,2,2-trifluoroethyl)-2H- | |
| | indazol-5-yl)-3,4- | |
| | dihydropyrido[2,3- | |
| | d]pyrimidin-2(1H)-one | |
| Example 121 | 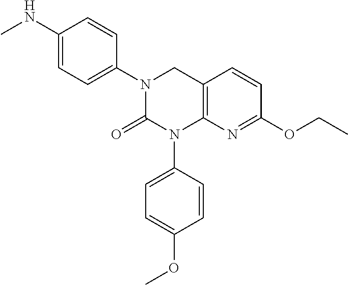 [SMILES: CCOc1ccc2c(n1)N(c1ccc(OC)cc1)C(=O)N(c1ccc(NC)cc1)C2] | LC-MS: m/z 405.2 (M + H)+. 1H NMR (400 MHz, DMSO-d₆) δ: 7.54 (d, J = 6.8 Hz, 1H), 7.18 (d, J = 7.6 Hz, 2H), 7.09 (d, J = 7.2 Hz, 2H), 6.96 (d, J = 6.8 Hz, 2H), 6.53 (d, J = 6.8 Hz, 2H), 6.36 (d, J = 7.6 Hz, 1H), 5.66 (s, 1H), 4.74 (s, 2H), 3.84 (q, J = 6.4 Hz, 2H), 3.78 (s, 3H), 2.67 (s, 3H), 1.06 (t, J = 6.4 Hz, 3H). |
| | 7-ethoxy-1-(4- | |
| | methoxyphenyl)-3-(4- | |
| | (methylamino)phenyl)-3,4- | |
| | dihydropyrido[2,3- | |
| | d]pyrimidin-2(1H)-one | |
| Example 122 | 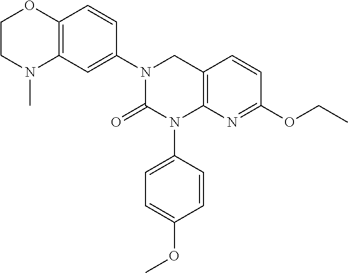 [SMILES: CCOc1ccc2c(n1)N(c1ccc(OC)cc1)C(=O)N(c1ccc3c(c1)N(C)CCO3)C2] | LC-MS: m/z 447.2 (M + H)+. 1H NMR (400 MHz, DMSO-d₆) δ: 7.54 (d, J = 8.4 Hz, 1H), 7.19 (d, J = 9.2 Hz, 2H), 6.97 (d, J = 9.2 Hz, 2H), 6.71 (d, J = 2.0 Hz, 1H), 6.66 (d, J = 8.0 Hz, 1H), 6.57 (dd, J = 8.4 Hz, 2.4 Hz, 1H), 6.37 (d, J = 8.0 Hz, 1H), 4.77 (s, 2H), 4.22 (t, J = 4.4 Hz, 2H), 3.85 (q, J = 6.8 Hz, 2H), 3.78 (s, 3H), 3.23 (t, J = 4.4 Hz, 2H), 2.82 (s, 3H), 1.06 (t, J = 6.4 Hz, 3H). |
| | 7-ethoxy-1-(4- | |
| | methoxyphenyl)-3-(4- | |
| | methyl-3,4-dihydro-2H- | |
| | benzo[b][1,4]oxazin-6-yl)- | |
| | 3,4-dihydropyrido[2,3- | |
| | d]pyrimidin-2(1H)-one | |
| Example 123 | 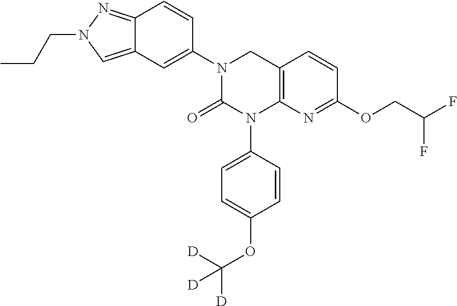 [SMILES: [2H]C([2H])([2H])Oc1ccc(N2C(=O)N(c3ccc4nn(CCC)cc4c3)Cc3ccc(OCC(F)F)nc32)cc1] | LC-MS: m/z 497.2 (M + H)+. 1H NMR (400 MHz, DMSO-d₆) δ: 8.38 (s, 1H), 7.69 (s, 1H), 7.65 (d, J = 8.4 Hz, 1H), 7.59 (d, J = 9.2 Hz, 1H), 7.29-7.22 (m, 3H), 6.99 (d, J = 8.8 Hz, 2H), 6.53 (d, J = 8.0 Hz, 1H), 6.02 (tt, J_{HF} = 55.6 Hz, 4.0 Hz, 1H), 4.93 (s, 2H), 4.38 (t, J = 6.8 Hz, 2H), 4.07 (td, J_{HF} = 14.4 Hz, 3.6 Hz, 2H), 2.01-1.86 (m, 2H), 0.86 (t, J = 7.2 Hz, 3H). |
| | 7-(2,2-difluoroethoxy)-1-(4- | |
| | methoxy-d3-phenyl)-3-(2- | |
| | propyl-2H-indazol-5-yl)- | |
| | 3,4-dihydropyrido[2,3- | |
| | d]pyrimidin-2(1H)-one | |
| Example 124 | 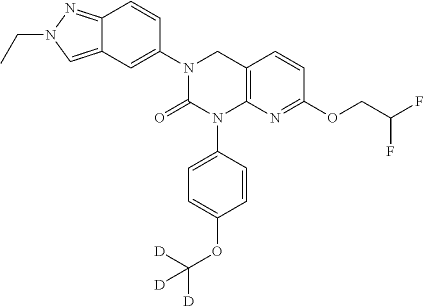 [SMILES: [2H]C([2H])([2H])Oc1ccc(N2C(=O)N(c3ccc4nn(CC)cc4c3)Cc3ccc(OCC(F)F)nc32)cc1] | LC-MS: m/z 483.2 (M + H)+. 1H NMR (400 MHz, DMSO-d₆) δ: 8.39 (s, 1H), 7.69 (d, J = 1.2 Hz, 1H), 7.66 (d, J = 8.0 Hz, 1H), 7.59 (d, J = 9.2 Hz, 1H), 7.29- 7.23 (m, 3H), 7.00 (d, J = 8.8 Hz, 2H), 6.53 (d, J = 8.4 Hz, 1H), 6.02 (tt, J_{HF} = 56.0 Hz, 4.0 Hz, 1H), 4.93 (s, 2H), 4.45 (t, J = 7.2 Hz, 2H), 4.07 (td, J_{HF} = 14.4 Hz, 4.0 Hz, 2H), 1.51 (t, J = 7.2 Hz, 3H). |
| | 7-(2,2-difluoroethoxy)-3-(2- | |
| | ethyl-2H-indazol-5-yl)-1-(4- | |
| | methoxy-d3-phenyl)-3,4- | |
| | dihydropyrido[2,3- | |
| | d]pyrimidin-2(1H)-one | |
| Example 125 | 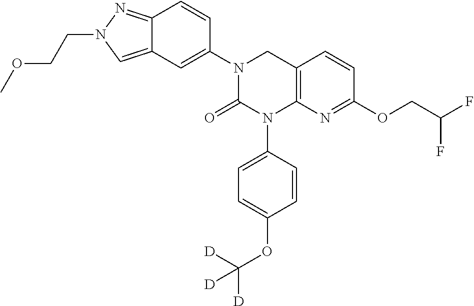 [SMILES: [2H]C([2H])([2H])Oc1ccc(N2C(=O)N(c3ccc4nn(CCOC)cc4c3)Cc3ccc(OCC(F)F)nc32)cc1] | LC-MS: m/z 512.2 (M + H)+. 1H NMR (400 MHz, DMSO-d₆) δ: 8.37 (s, 1H), 7.70 (d, J = 1.6 Hz, 1H), 7.66 (d, J = 8.0 Hz, 1H), 7.59 (d, J = 9.2 Hz, 1H), 7.30- 7.22 (m, 3H), 7.00 (d, J = 9.2 Hz, 2H), 6.53 (d, J = 8.4 Hz, 1H), 6.02 (tt, J_{HF} = 55.6 Hz, 4.0 Hz, 1H), 4.93 (s, 2H), 4.58 (t, J = 5.6 Hz, 2H), 4.07 (td, J_{HF} = 14.4 Hz, 4.0 Hz, 2H), 3.82 (t, J = 5.6 Hz, 2H), 3.23 (s, 3H). |
| | 7-(2,2-difluoroethoxy)-3-(2- | |
| | (2-methoxyethyl)-2H- | |
| | indazol-5-yl)-1-(4-methoxy- | |
| | d3-phenyl)-3,4- | |
| | dihydropyrido[2,3- | |
| | d]pyrimidin-2(1H)-one | |
| Example 126 | 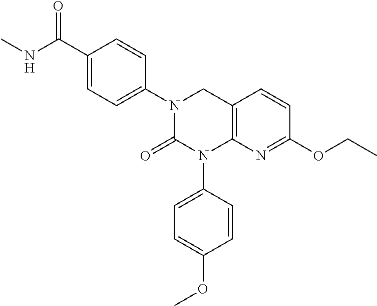 [SMILES: CCOc1ccc2c(n1)N(c1ccc(OC)cc1)C(=O)N(c1ccc(C(=O)NC)cc1)C2] | LC-MS: m/z 433.2 (M + H)+. 1H NMR (400 MHz, DMSO-d₆) δ: 8.43 (q, J = 4.8 Hz, 1H), 7.85 (d, J = 8.4 Hz, 2H), 7.60 (d, J = 8.0 Hz, 2H), 7.49 (d, J = 8.8 Hz, 2H), 7.22 (d, J = 8.8 Hz, 2H), 6.99 (d, J = 8.8 Hz, 2H), 6.41 (d, J = 8.0 Hz, 1H), 4.93 (s, 2H), 3.87 (q, J = 7.2 Hz, 2H), 3.80 (s, 3H), 2.78 (d, J = 4.8 Hz, 3H), 1.07 (t, J = 7.2 Hz, 3H). |
| | 4-(7-ethoxy-1-(4- | |
| | methoxyphenyl)-2-oxo-1,2- | |
| | dihydropyrido[2,3- | |
| | d]pyrimidin-3(4H)-yl)-N- | |
| | methylbenzamide | |
| Example 127 | 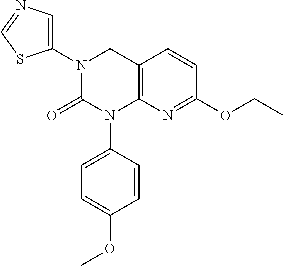 [SMILES: CCOc1ccc2c(n1)N(c1ccc(OC)cc1)C(=O)N(c1cncs1)C2] | LC-MS: m/z 383.1 (M + H)+. 1H NMR (400 MHz, DMSO-d₆) δ: 8.68 (s, 1H), 7.74 (s, 1H), 7.65 (d, J = 8.8 Hz, 1H), 7.23 (d, J = 8.8 Hz, 2H), 7.01 (d, J = 8.8 Hz, 2H), 6.48 (d, J = 8.0 Hz, 1H), 5.08 (s, 2H), 3.84 (q, J = 6.8 Hz, 2H), 3.80 (s, 3H), 1.05 (t, J = 6.8 Hz, 3H). |
| | ethoxy-1-(4- | |
| | methoxyphenyl)-3-(thiazol- | |
| | 5-yl)-3,4-dihydropyrido[2,3- | |
| | d]pyrimidin-2(1H)-one | |
| Example 128 | 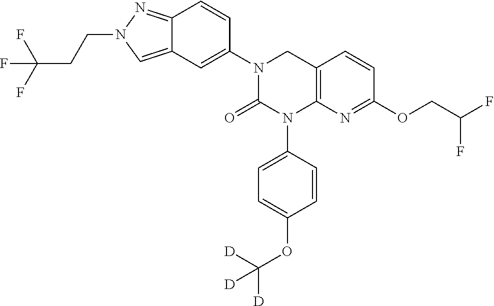 [SMILES: [2H]C([2H])([2H])Oc1ccc(N2C(=O)N(c3ccc4nn(CCC(F)(F)F)cc4c3)Cc3ccc(OCC(F)F)nc32)cc1] | LC-MS: m/z 551.2 (M + H)+. 1H NMR (400 MHz, DMSO-d₆) δ: 8.48 (s, 1H), 7.71 (d, J = 1.6 Hz, 1H), 7.65 (d, J = 8.0 Hz, 1H), 7.60 (d, J = 9.2 Hz, 1H), 7.30 (dd, J = 9.2 Hz, 2.0 Hz, 1H), 7.26 (d, J = 8.4 Hz, 2H), 7.00 (d, J = 8.8 Hz, 2H), 6.53 (d, J = 8.0 Hz, 1H), 6.02 (tt, J_{HF} = 55.6 Hz, 4.0 Hz, 1H), 4.94 (s, 2H), 4.72 (t, J = 6.8 Hz, 2H), 4.07 (td, J_{HF} = 14.4 Hz, 4.0 Hz, 2H), 3.12-2.98 (m, 2H). |
| | 7-(2,2-difluoroethoxy)-1-(4- | |
| | methoxy-d3-phenyl)-3-(2- | |
| | (3,3,3-trifluoropropyl)-2H- | |
| | indazol-5-yl)-3,4- | |
| | dihydropyrido[2,3- | |
| | d]pyrimidin-2(1H)-one | |
| Example 129 | 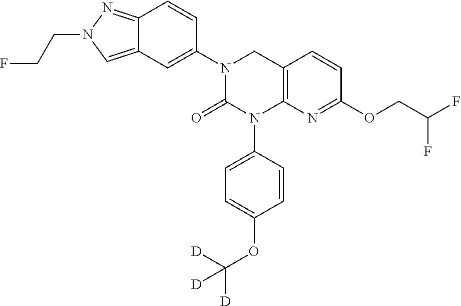 [SMILES: [2H]C([2H])([2H])Oc1ccc(N2C(=O)N(c3ccc4nn(CCF)cc4c3)Cc3ccc(OCC(F)F)nc32)cc1] | LC-MS: m/z 501.2 (M + H)+. 1H NMR (400 MHz, DMSO-d₆) δ: 8.43 (s, 1H), 7.72 (s, 1H), 7.66 (d, J = 8.0 Hz, 1H), 7.61 (d, J = 9.2 Hz, 1H), 7.30 (dd, J = 9.2 Hz, 2.0 Hz, 1H), 7.26 (d, J = 8.4 Hz, 2H), 7.00 (d, J = 8.8 Hz, 2H), 6.53 (d, J = 8.4 Hz, 1H), 6.01 (tt, J_{HF} = 55.6 Hz, 4.0 Hz, 1H), 4.97 (t, J = 4.4 Hz, 1H), 4.93 (s, 2H), 4.85 (t, J = 4.4 Hz, 1H), 4.79 (t, J = 4.4 Hz, 1H), 4.73 (t, J = 4.4 Hz, 1H), 4.07 (td, J_{HF} = 14.4 Hz, 4.0 Hz, 2H). |
| | 7-(2,2-difluoroethoxy)-3-(2- | |
| | (2-fluoroethyl)-2H-indazol- | |
| | 5-yl)-1-(4-methoxy-d3- | |
| | phenyl)-3,4- | |
| | dihydropyrido[2,3- | |
| | d]pyrimidin7-2(1H)-one | |
| Example 130 | 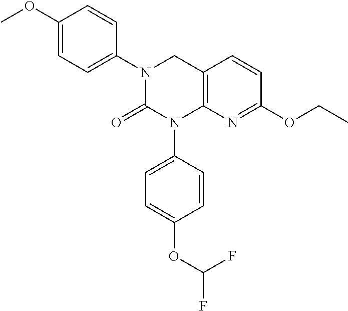 [SMILES: CCOc1ccc2c(n1)N(c1ccc(OC(F)F)cc1)C(=O)N(c1ccc(OC)cc1)C2] | LC-MS: m/z 442.2 (M + H)+. 1H NMR (400 MHz, DMSO-d₆) δ: 7.58 (d, J = 8.0 Hz, 1H), 7.37 (d, J = 8.8 Hz, 2H), 7.33 (d, J = 9.2 Hz, 2H), 7.29 (t, J_{HF} = 74.0 Hz, 1H), 7.23 (d, J = 8.8 Hz, 2H), 6.96 (d, J = 8.8 Hz, 2H), 6.40 (d, J = 8.0 Hz, 1H), 4.83 (s, 2H), 3.85 (q, J = 7.2 Hz, 2H), 3.76 (s, 3H), 1.05 (t, J = 7.2 Hz, 3H). |
| | 1-(4- | |
| | (difluoromethoxy)phenyl)- | |
| | 7-ethoxy-3-(4- | |
| | methoxyphenyl)-3,4- | |
| | dihydropyrido[2,3- | |
| | d]pyrimidin-2(1H)-one | |
| Example 401 | 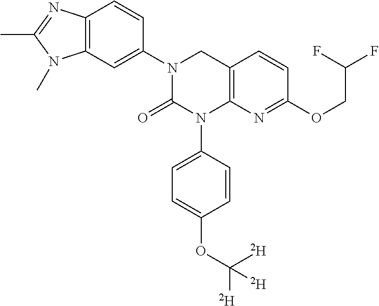 [SMILES: [2H]C([2H])([2H])Oc1ccc(N2C(=O)N(c3ccc4nc(C)n(C)c4c3)Cc3ccc(OCC(F)F)nc32)cc1] | LC-MS: m/z 483 (M + H)+. 1H NMR (400 MHz, DMSO-d₆) δ: 7.66 (d, J = 8.1 Hz, 1H), 7.55 (d, J = 1.8 Hz, 1H), 7.50 (d, J = 8.5 Hz, 1H), 7.29-7.21 (m, 2H), 7.18 (dd, J = 8.5 Hz, 2.0 Hz, 1H), 7.04-6.96 (m, 2H), 6.53 (d, J = 8.0 Hz, 1H), 6.01 (tt, J_{HF} = 55.6 Hz, J = 3.9 Hz, 1H), 4.93 (s, 2H), 4.07 (td, J_{HF} = 14.4 Hz, J = 3.9 Hz, 2H), 3.72 (s, 3H), 2.52 (s, 3H). |
| | 7-(2,2-difluoroethoxy)-3- | |
| | (1,2-dimethyl-1H- | |
| | benzo[d]imidazol-6-yl)-1- | |
| | (4-(methoxy-d3)phenyl)- | |
| | 3,4-dihydropyrido[2,3- | |
| | d]pyrimidin-2(1H)-one | |
| Example 402 | 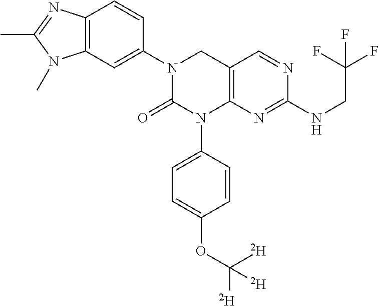 [SMILES: [2H]C([2H])([2H])Oc1ccc(N2C(=O)N(c3ccc4nc(C)n(C)c4c3)Cc3cnc(NCC(F)(F)F)nc32)cc1] | LC-MS: m/z 501 (M + H)+. 1H NMR (400 MHz, DMSO-d₆) δ: 8.13 (d, J = 4.4 Hz, 1H), 7.65 (d, J = 1.6 Hz, 1H), 7.52-7.58 (m, 2H), 7.26 (dd, J = 8.4 Hz, 1.6 Hz, 1H), 7.21 (d, J = 8.8 Hz, 2H), 6.98 (d, J = 8.8 Hz, 2H), 4.84 (s, 2H), 4.02-3.87 (m, 2H), 3.75 (s, 3H), 2.58 (s, 3H). |
| | 3-(1,2-dimethyl-1H- | |
| | benzo[d]imidazol-6-yl)-1- | |
| | (4-(methoxy-d3)phenyl)-7- | |
| | ((2,2,2- | |
| | trifluoroethyl)amino)-3,4- | |
| | dihydropyrimido[4,5- | |
| | d]pyrimidin-2(1H)-one | |

### General Procedure II

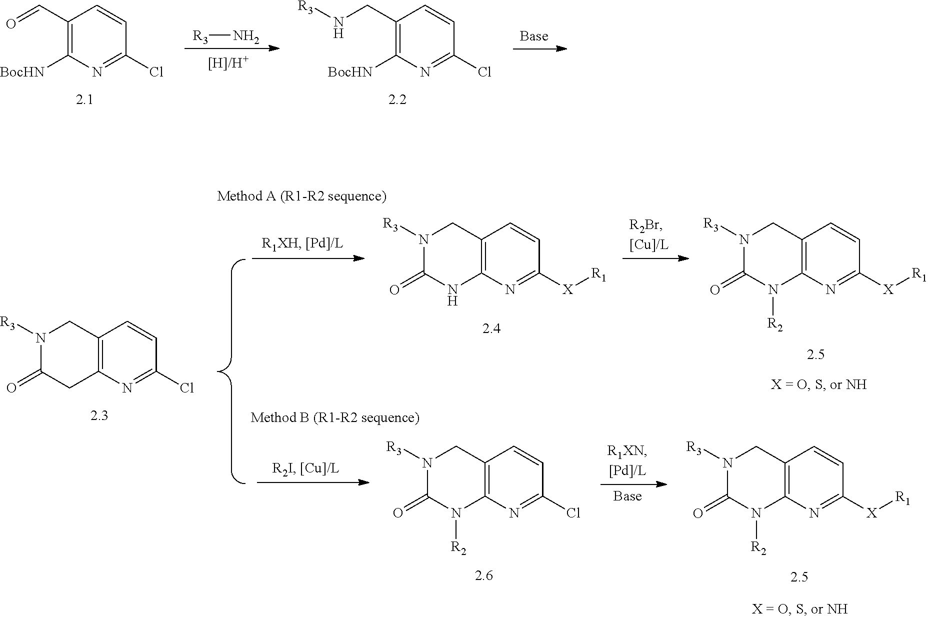

Compounds of structure 2.5 were obtained through the scheme depicted as General Procedure II. Beginning with aldehyde 2.1, the desired R₃ group was introduced using a reductive amination to afford amine 2.2. Amine 2.2 was then cyclized to urea 2.3 under basic conditions. The desired R₁ and R₂ groups were then introduced either by a palladium mediated C—X coupling to generate compound 2.4 followed by a copper mediated C—N coupling to afford compound 2.5 (Method A), or by a copper mediated C—N coupling to generate compound 2.6 followed by a palladium mediated C—X coupling to afford 2.5 (Method B).

### Preparation of Example 131 Via General Procedure II (Method A)

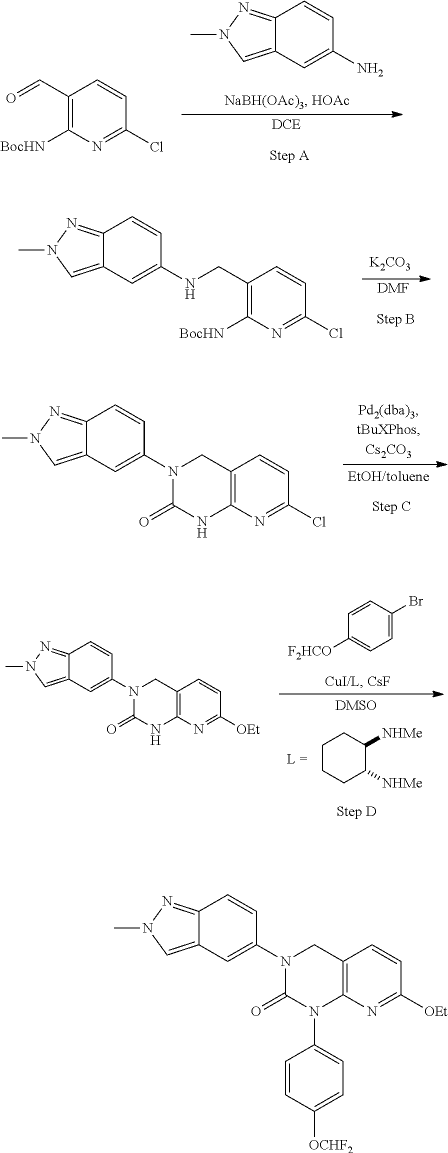

### Step A: tert-butyl (6-chloro-3-(((2-methyl-2H-indazol-5-yl)amino)methyl)pyridin-2-yl)carbamate

To a solution of tert-butyl (6-chloro-3-formylpyridin-2-yl)carbamate (commercially available) (3.8 g, 15 mmol, 1.0 eq.) and 2-methyl-2H-indazol-5-amine (2.0 g, 14 mmol, 0.93 eq.) in DCE (50 mL) was added AcOH (3.26 g, 54 mmol, 3.6 eq.), the reaction mixture was stirred at room temperature for 5 hrs. Then the reaction mixture was cooled to 0° C., NaBH(OAc)₃ (8.64 g, 41 mmol, 2.7 eq.) was added in several portions. After addition, the mixture was allowed to warm to room temperature and stirred for additional 16 hrs. The reaction quenched with ice-cooled NaHCO₃ (sat. aq.) (30 mL), extracted with EtOAc (50 mL×3), the combined organic layers were washed with brine (30 mL) and dried over Na₂SO₄, concentrated under reduced pressure and purified by flash column chromatography on silica gel to afford tert-butyl (6-chloro-3-(((2-methyl-2H-indazol-5-yl)amino)methyl)pyridin-2-yl)carbamate (3.2 g, 61% yield) as a pale green solid. LC-MS (ESI): m/z 388 [M+H]⁺.

### Step B: 7-chloro-3-(2-methyl-2H-indazol-5-yl)-3,4-dihydropyrido[2,3-d]pyrimidin-2(1H)-on

A mixture of tert-butyl (6-chloro-3-(((2-methyl-2H-indazol-5-yl)amino)methyl)pyridin-2-yl)carbamate (3.2 g, 8.25 mmol, 1.0 eq.) and K₂CO₃ (11.4 g, 82.5 mmol, 10.0 eq.) in dioxane (40 mL) was stirred at 100° C. for 16 hrs. The progress of the reaction was monitored by LC-MS, after completion, the reaction mixture was quenched with ice water (50 mL), the precipitate collected and dried under reduced pressure to afford 7-chloro-3-(2-methyl-2H-indazol-5-yl)-3,4-dihydropyrido[2,3-d]pyrimidin-2(1H)-one (2.2 g, 85% yield) as a white solid. LC-MS (ESI): m/z 314 [M+H]⁺.

### Step C: 7-ethoxy-3-(2-methyl-2H-indazol-5-yl)-3,4-dihydropyrido[2,3-d]pyrimidin-2(1H)-one

A mixture of 7-chloro-3-(2-methyl-2H-indazol-5-yl)-3,4-dihydropyrido[2,3-d]pyrimidin-2(1H)-one (2.2 g, 7.0 mmol, 1.0 eq.), Cs₂CO₃ (6.86 g, 21 mmol, 3.0 eq.), Pd₂(dba)₃ (0.64 g, 0.70 mmol, 0.1 eq.) and t-BuXPhos (0.6 g, 1.4 mmol, 0.2 eq.) in EtOH (300 mL) and toluene (30 mL) was stirred at 80° C. under N₂ atmosphere for 16 hrs. The progress of the reaction was monitored by LC-MS, after completion, the reaction mixture was filtered through a short pad of Celite®, concentrated under reduced pressure, the residue was purified by flash column chromatography on silica gel to afford 7-ethoxy-3-(2-methyl-2H-indazol-5-yl)-3,4-dihydropyrido[2,3-d]pyrimidin-2(1H)-one, as a white solid (2.0 g, 88% yield). LC-MS (ESI): m/z 324 [M+H]⁺.

### Step D: 1-(4-(difluoromethoxy)phenyl)-7-ethoxy-3-(2-methyl-2H-indazol-5-yl)-3,4-dihydropyrido[2,3-d]pyrimidin-2(1H)-one

A mixture of 7-ethoxy-3-(2-methyl-2H-indazol-5-yl)-3,4-dihydropyrido[2,3-d]pyrimidin-2(1H)-one (50 mg, 0.15 mmol, 1.0 eq.), 1-bromo-4-(difluoromethoxy)benzene (35 mg, 0.15 mmol, 1.0 eq.), CsF (70 mg, 0.45 mmol, 3.0 eq.), CuI (29 mg, 0.15 mmol, 1.0 eq.) and N¹,N²-dimethylcyclohexane-1,2-diamine (44 mg, 0.3 mmol, 2.0 eq.) in DMSO (3 mL) was stirred at 100° C. under N₂ atmosphere for 16 hrs. the reaction mixture was diluted with H₂O (10 mL), washed with EtOAc (10 mL×3), the combined organic layers were washed with brine (10 mL) and dried over Na₂SO₄, concentrated under reduced pressure, the residue was purified by RP-prep-HPLC to afford 1-(4-(difluoromethoxy)phenyl)-7-ethoxy-3-(2-methyl-2H-indazol-5-yl)-3,4-dihydropyrido[2,3-d]pyrimidin-2(1H)-one (Example 131).

¹H NMR (400 MHz, DMSO-d₆) δ (ppm): 8.35 (s, 1H), 7.69 (d, J=1.6 Hz, 1H), 7.59 (t, J=8.4 Hz, 2H), 7.39 (t, J=8.8 Hz, 2H), 7.30 (t, J_{HF}=74 Hz, 1H), 7.28 (dd, J=9.6 Hz, 2.0 Hz, 1H), 7.24 (d, J=8.8 Hz, 2H), 6.42 (d, J=8.0 Hz, 1H), 4.91 (s, 2H), 4.17 (s, 3H), 3.86 (q, J=7.2 Hz, 2H), 1.06 (t, J=7.2 Hz, 3H).

LC-MS (ESI): m/z 466 [M+H]⁺.

### Preparation of Example 132 via General Procedure II (method B)

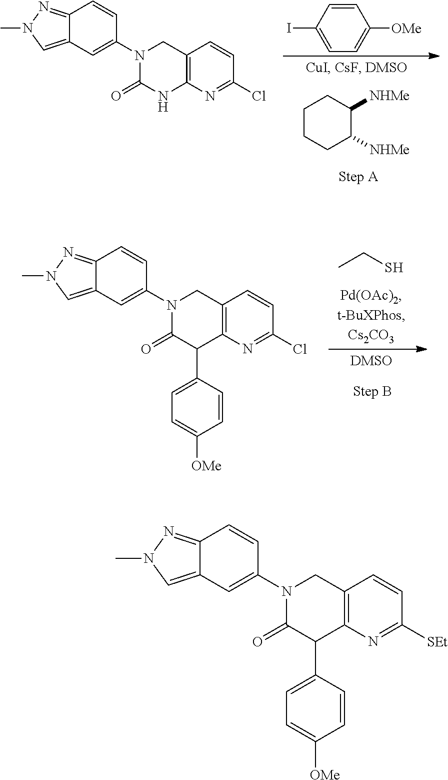

### Step A: 7-chloro-1-(4-methoxyphenyl)-3-(2-methyl-2H-indazol-5-yl)-3,4-dihydropyrido[2,3-d]pyrimidin-2(1H)-one

To a solution of 7-chloro-3-(2-methyl-2H-indazol-5-yl)-3,4-dihydropyrido[2,3-d]pyrimidin-2(1H)-one (1.0 g, 3.19 mmol, 1.0 eq.) in DMSO (15 mL) was added 1-iodo-4-methoxybenzene (0.90 g, 3.82 mmol, 1.2 eq.), CuI (0.61 g, 3.19 mmol, 1.0 eq.), CsF (1.45 g, 9.56 mmol, 3.0 eq.) and N¹,N²-dimethylcyclohexane-1,2-diamine (0.91 g, 6.37 mmol, 2.0 eq.), the reaction mixture was stirred at 100° C. under N₂ atmosphere for 16 hrs. The progress was monitored by LC-MS, after completion, the reaction was diluted with H₂O (20 mL), extracted with EtOAc (40 mL×3), dried over Na₂SO₄, concentrated under reduced pressure, the residue was purified by flash column chromatography on silica gel to afford 7-chloro-1-(4-methoxyphenyl)-3-(2-methyl-2H-indazol-5-yl)-3,4-dihydropyrido[2,3-d]pyrimidin-2(1H)-one (900 mg, 57% yield) as a white solid. LC-MS (ESI): m/z 420 [M+H]⁺.

### Step B: 7-(ethylthio)-1-(4-methoxyphenyl)-3-(2-methyl-2H-indazol-5-yl)-3,4-dihydropyrido[2,3-d]pyrimidin-2(1H)-one

A mixture of 7-chloro-1-(4-methoxyphenyl)-3-(2-methyl-2H-indazol-5-yl)-3,4-dihydropyrido[2,3-d]pyrimidin-2(1H)-one (50 mg, 0.12 mmol, 1.0 eq.), Pd(OAc)₂ (3 mg, 0.012 mmol, 0.1 eq.), t-BuXPhos (10 mg, 0.024 mmol, 0.2 eq.) and Cs₂CO₃ (116 mg, 0.36 mmol, 3.0 eq.) in DMSO (3 mL), the system was degassed with nitrogen, then ethanethiol (0.3 mL) was added via a syringe, the reaction was carried out in sealed tube and stirred at 70° C. for 16 hrs. The progress was monitored by LC-MS, after completion, the reaction was diluted with H₂O (20 mL), extracted with EtOAc (40 mL×3), dried over Na₂SO₄, concentrated under reduced pressure and purified by RP-prep-HPLC to afford 7-(ethylthio)-1-(4-methoxyphenyl)-3-(2-methyl-2H-indazol-5-yl)-3,4-dihydropyrido[2,3-d]pyrimidin-2(1H)-one (Example 132).

¹H NMR (400 MHz, DMSO-d₆) δ (ppm): 8.35 (s, 1H), 7.69 (s, 1H), 7.57 (d, J=9.2 Hz, 1H), 7.51 (d, J 7.6 Hz, 1H), 7.27 (d, J 8.8 Hz, 1H), 7.23 (d, J=8.8 Hz, 2H), 7.00 (d, J=8.8 Hz, 2H), 6.88 (d, J=7.6 Hz, 1H), 4.93 (s, 2H), 4.17 (s, 3H), 3.79 (s, 3H), 2.64 (q, J=7.2 Hz, 2H), 0.92 (6, J=7.2 Hz, 3H).

LC-MS (ESI): m/z 446 [M+H]⁺.

The procedure set forth above for General Procedure II (Method A) was used to synthesize the following compounds by using appropriate starting materials:

| Cpd No. | Structure | Characterization |
|---|---|---|
| Example 133 | 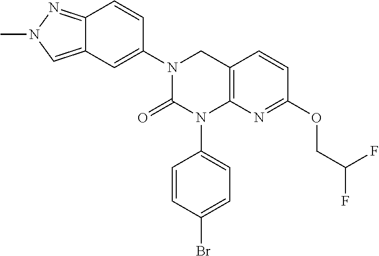 [SMILES: Cn1cc2cc(N3Cc4ccc(OCC(F)F)nc4N(c4ccc(Br)cc4)C3=O)ccc2n1] | LC-MS (ESI): m/z 514.2, 516.2 [M + H]+. 1H NMR (400 MHz, DMSO-d₆) δ: 8.35 (s, 1H), 7.70-7.63 (m, 4H), 7.58 (d, J = 8.8 Hz, 1H), 7.35 (d, J = 8.4 Hz, 2H), 7.28 (dd, J = 9.2 Hz, 2.0 Hz, 1H), 6.56 (d, J = 8.0 Hz, 1H), 6.05 (tt, J_{HF} = 55.2 Hz, J = 3.6 Hz, 1H), 4.94 (s, 2H), 4.17 (s, 3H), 4.08 (td, J_{HF} = 14.4 Hz, J = 3.6 Hz, 2H). |
| | 1-(4-bromophenyl)-7-(2,2- | |
| | difluoroethoxy)-3-(2-methyl-2H- | |
| | indazol-5-yl)-3,4- | |
| | dihydropyrido[2,3-d]pyrimidin- | |
| | 2(1H)-one | |
| Example 135 | 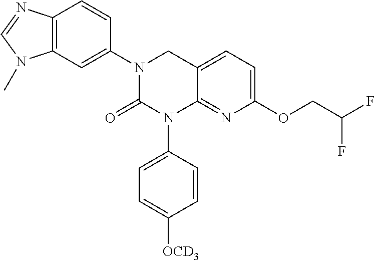 | LC-MS (ESI): m/z 469.2 [M + H]+. 1H NMR (400 MHz, DMSO-d₆) δ: 8.22 (s, 1H), 7.66 (t, J = 7.7 Hz, 3H), 7.27 (d, J = 8.8 Hz, 3H), 7.01 (d, J = 8.8 Hz, 2H), 6.54 (d, J = 8.0 Hz, 1H), 6.03 (tt, J_{HF} = 55.6 Hz, J = 3.8 Hz, 1H), 4.96 (s, 2H), 4.08 (td, J_{HF} = 14.6 Hz, J = 3.8 Hz, 2H), 3.84 (s, 3H). |
| | 7-(2,2-difluoroethoxy)-1-(4- | |
| | (methoxy-d3)phenyl)-3-(1-methyl- | |
| | 1H-benzo[d]imidazol-6-yl)-3,4- | |
| | dihydropyrido[2,3-d]pyrimidin- | |
| | 2(1H)-one | |
| Example 136 | 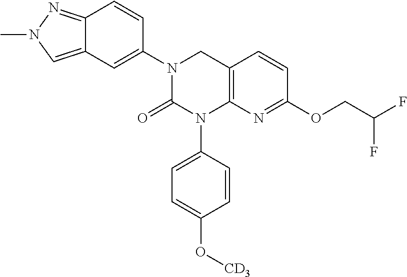 [SMILES: [2H]C([2H])([2H])Oc1ccc(N2C(=O)N(c3ccc4nn(C)cc4c3)Cc3ccc(OCC(F)F)nc32)cc1] | LC-MS (ESI): m/z 469.2 [M + H]+. 1H NMR (400 MHz, DMSO-d₆) δ: 8.34 (s, 1H), 7.69 (d, J = 1.5 Hz, 1H), 7.66 (d, J = 8.1 Hz, 1H), 7.57 (d, J = 9.2 Hz, 1H), 7.30-7.23 (m, 3H), 7.00 (d, J = 8.8 Hz, 2H), 6.53 (d, J = 8.0 Hz, 1H), 6.02 (tt, J_{HF} = 55.4 Hz, J = 3.9 Hz, 1H), 4.93 (s, 2H), 4.17 (s, 3H), 4.07 (td, J_{HF} = 14.5, J = 3.9 Hz, 2H). |
| | 7-(2,2-difluoroethoxy)-1-(4- | |
| | (methoxy-d3)phenyl)-3-(2-methyl- | |
| | 2H-indazol-5-yl)-3,4- | |
| | dihydropyrido[2,3-d]pyrimidin- | |
| | 2(1H)-one | |
| Example 137 | 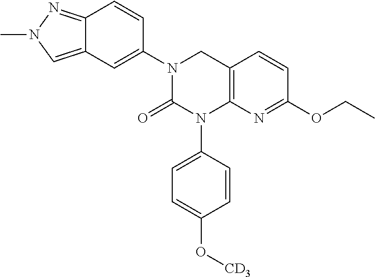 [SMILES: [2H]C([2H])([2H])Oc1ccc(N2C(=O)N(c3ccc4nn(C)cc4c3)Cc3ccc(OCC)nc32)cc1] | LC-MS (ESI): m/z 433.0 [M + H]+. 1H NMR (400 MHz, DMSO-d₆) δ: 8.40 (s, 1H), 7.74 (d, J = 1.6 Hz, 1H), 7.65 (d, J = 6.0 Hz, 1H), 7.63 (d, J = 7.2 Hz, 1H), 7.33 (dd, J = 9.2 Hz, 2.0 Hz, 1H), 7.29 (d, J = 8.8 Hz, 2H), 7.04 (d, J = 8.8 Hz, 2H), 6.46 (d, J = 8.0 Hz, 1H), 4.96 (s, 2H), 4.23 (s, 3H), 3.93 (q, J = 7.2 Hz, 2H), 1.13 (t, J = 7.2 Hz, 3H). |
| | 7-ethoxy-1-(4-(methoxy- | |
| | d3)phenyl)-3-(2-methyl-2H- | |
| | indazol-5-yl)-3,4- | |
| | dihydropyrido[2,3-d]pyrimidin- | |
| | 2(1H)-one | |
| Example 138 | 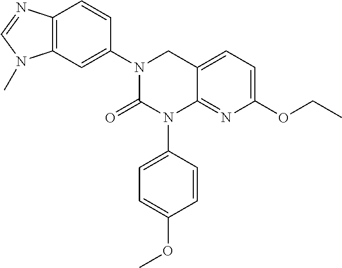 [SMILES: CCOc1ccc2c(n1)N(c1ccc(OC)cc1)C(=O)N(c1ccc3ncn(C)c3c1)C2] | LC-MS (ESI): m/z 430.0 [M + H]+. 1H NMR (400 MHz, DMSO-d₆) δ: 8.20 (s, 1H), 7.65 (d, J = 7.6 Hz, 2H), 7.58 (d, J = 8.0 Hz, 1H), 7.26 (d, J = 2.0 Hz, 1H), 7.23 (d, J = 8.8 Hz, 2H), 6.98 (d, J = 8.8 Hz, 2H), 6.40 (d, J = 8.0 Hz, 1H), 4.92 (s, 2H), 3.85 (q, J = 7.0 Hz, 2H), 3.83 (s, 3H), 3.79 (s, 3H), 1.07 (t, J = 7.0 Hz, 3H). |
| | 7-ethoxy-1-(4-methoxyphenyl)-3- | |
| | (1-methyl-1H-benzo[d]imidazol-6- | |
| | yl)-3,4-dihydropyrido[2,3- | |
| | d]pyrimidin-2(1H)-one | |
| Example 139 | 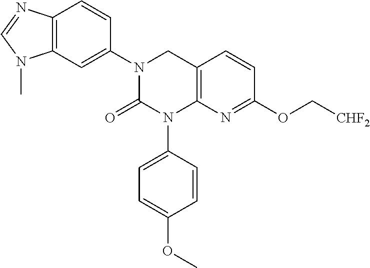 [SMILES: COc1ccc(N2C(=O)N(c3ccc4ncn(C)c4c3)Cc3ccc(OCC(F)F)nc32)cc1] | LC-MS (ESI): m/z 466.0 [M + H]+. 1H NMR (400 MHz, DMSO-d₆) δ: 8.21 (s, 1H), 7.70-7.62 (m, 3H), 7.30-7.23 (m, 3H), 7.03-6.97 (m, 2H), 6.53 (d, J = 8.0 Hz, 1H), 6.02 (tt, J_{HF} = 56.0 Hz, J = 3.6 Hz, 1H), 4.95 (s, 2H), 4.08 (td, J_{HF} = 14.4 Hz, J = 3.6 Hz, 2H), 3.83 (s, 3H), 3.79 (s, 3H). |
| | 7-(2,2-difluoroethoxy)-1-(4- | |
| | methoxyphenyl)-3-(1-methyl-1H- | |
| | benzo[d]imidazol-6-yl)-3,4- | |
| | dihydropyrido[2,3-d]pyrimidin- | |
| | 2(1H)-one | |
| Example 140 | 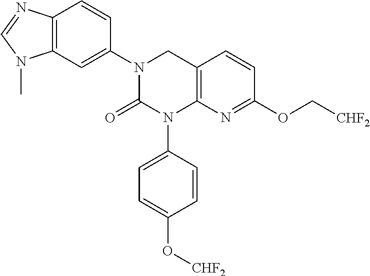 [SMILES: Cn1cnc2ccc(N3Cc4ccc(OCC(F)F)nc4N(c4ccc(OC(F)F)cc4)C3=O)cc21] | LC-MS (ESI): m/z 502.0 [M + H]+. 1H NMR (400 MHz, DMSO-d₆) δ: 8.22 (s, 1H), 7.72-7.62 (m, 3H), 7.45-7.40 (m, 2H), 7.29-7.26 (m, 3H), 7.28 (t, J_{HF} = 74.0 Hz, 1H), 6.56 (d, J = 8.0 Hz, 1H), 6.03 (tt, J_{HF} = 56.0 Hz, J = 4.0 Hz, 1H), 4.97 (s, 2H), 4.07 (td, J_{HF} = 14.8 Hz, J = 4.0 Hz, 2H), 3.83 (s, 3H). |
| | 7-(2,2-difluoroethoxy)-1-(4- | |
| | (difluoromethoxy)phenyl)-3-(1- | |
| | methyl-1H-benzo[d]imidazol-6- | |
| | yl)-3,4-dihydropyrido[2,3- | |
| | d]pyrimidin-2(1H)-one | |
| Example 141 | 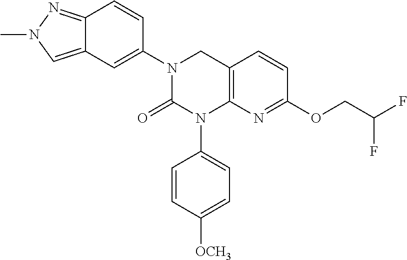 [SMILES: COc1ccc(N2C(=O)N(c3ccc4nn(C)cc4c3)Cc3ccc(OCC(F)F)nc32)cc1] | LC-MS (ESI): m/z 466.0 [M + H]+. 1H NMR (400 MHz, DMSO-d₆) δ: 8.35 (s, 1H), 7.69 (d, J = 1.6 Hz, 1H), 7.66 (d, J = 8.0 Hz, 1H), 7.57 (d, J = 9.2 Hz, 1H), 7.30-7.21 (m, 3H), 7.00 (d, J = 8.8 Hz, 2H), 6.53 (d, J = 8.0 Hz, 1H), 6.01 (tt, J_{HF} = 55.6 Hz, J = 4.0 Hz, 1H), 4.93 (s, 2H), 4.17 (s, 3H), 4.07 (td, J_{HF} = 14.4 Hz, J = 4.0 Hz, 2H), 3.79 (s, 3H). |
| | 7-(2,2-difluoroethoxy)-3-(2- | |
| | methyl-2H-indazol-5-yl)-1-(4- | |
| | (trifluoromethoxy)phenyl)-3,4- | |
| | dihydropyrido[2,3-d]pyrimidin- | |
| | 2(1H)-one | |
| Example 142 | 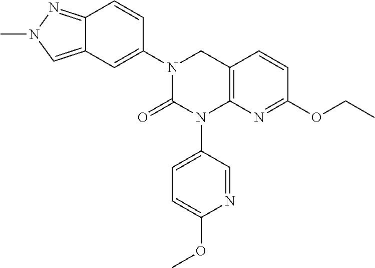 [SMILES: CCOc1ccc2c(n1)N(c1ccc(OC)nc1)C(=O)N(c1ccc3nn(C)cc3c1)C2] | LC-MS (ESI): m/z 431.0 [M + H]+. 1H NMR (400 MHz, DMSO-d₆) δ: 8.35 (s, 1H), 8.23 (d, J = 5.6 Hz, 1H), 7.70 (d, J = 1.6 Hz, 1H), 7.62 (d, J = 8.0 Hz, 1H), 7.58 (d, J = 9.2 Hz, 1H), 7.27 (dd, J = 9.2 Hz, 2.0 Hz, 1H), 7.01 (dd, J = 5.6 Hz, 1.6 Hz, 1H), 6.88 (s, 1H), 6.46 (d, J = 8.4 Hz, 1H), 4.91 (s, 2H), 4.17 (s, 3H), 3.90 (q, J = 6.8 Hz, 2H), 3.89 (s, 3H), 1.1 (t, J = 6.8 Hz, 3H). |
| | 7-ethoxy-1-(6-methoxypyridin-3- | |
| | yl)-3-(2-methyl-2H-indazol-5-yl)- | |
| | 3,4-dihydropyrido[2,3- | |
| | d]pyrimidin-2(1H)-one | |
| Example 143 | 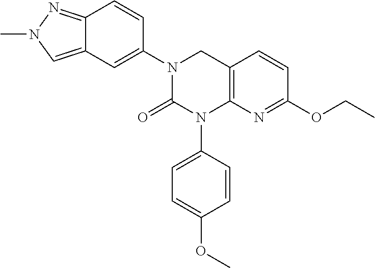 [SMILES: CCOc1ccc2c(n1)N(c1ccc(OC)cc1)C(=O)N(c1ccc3nn(C)cc3c1)C2] | LC-MS (ESI): m/z 430.0 [M + H]+. 1H NMR (400 MHz, DMSO-d₆) δ: 8.35 (s, 1H), 7.69 (d, J = 1.6 Hz, 1H), 7.59 (d, J = 6.0 Hz, 1H), 7.57 (d, J = 6.8 Hz, 1H), 7.27 (dd, J = 9.2 Hz, 2.0 Hz, 1H), 7.23 (d, J = 8.8 Hz, 2H), 6.99 (d, J = 8.8 Hz, 2H), 6.40 (d, J = 8.0 Hz, 1H), 4.90 (s, 2H), 4.17 (s, 3H), 3.87 (q, J = 6.8 Hz, 2H), 3.80 (s, 3H), 1.08 (t, J = 6.8 Hz, 3H). |
| | 7-ethoxy-1-(4-methoxyphenyl)-3- | |
| | (2-methyl-2H-indazol-5-yl)-3,4- | |
| | dihydropyrido[2,3-d]pyrimidin- | |
| | 2(1H)-one | |
| Example 144 | 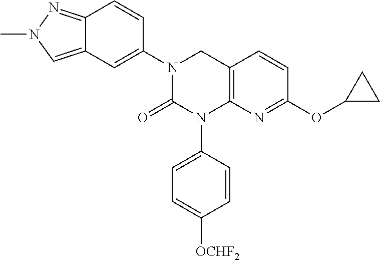 [SMILES: Cn1cc2cc(N3Cc4ccc(OC5CC5)nc4N(c4ccc(OC(F)F)cc4)C3=O)ccc2n1] | LC-MS (ESI): m/z 478.2 [M + H]+. 1H NMR (400 MHz, DMSO-d₆) δ: 8.36 (s, 1H), 7.70 (d, J = 2.0 Hz, 1H), 7.66 (d, J = 8.0 Hz, 1H), 7.58 (d, J = 9.2 Hz, 1H), 7.38 (d, J = 8.8 Hz, 2H), 7.31-7.22 (m, 3H), 7.27 (t, J_{HF} = 74.0 Hz, 1H), 6.58 (d, J = 8.0 Hz, 1H), 4.94 (s, 2H), 4.18 (s, 3H), 3.72-3.68 (m, 1H), 0.58-0.53 (m, 4H). |
| | 7-cyclopropoxy-1-(4- | |
| | (difluoromethoxy)phenyl)-3-(2- | |
| | methyl-2H-indazol-5-yl)-3,4- | |
| | dihydropyrido[2,3-d]pyrimidin- | |
| | 2(1H)-one | |
| Example 145 | 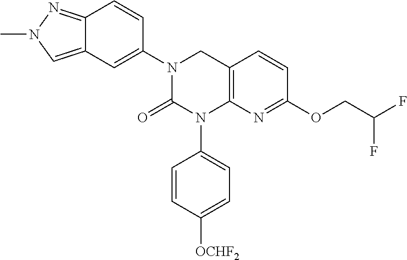 [SMILES: Cn1cc2cc(N3Cc4ccc(OCC(F)F)nc4N(c4ccc(OC(F)F)cc4)C3=O)ccc2n1] | LC-MS (ESI): m/z 502.2 [M + H]+. 1H NMR (400 MHz, DMSO-d₆) δ: 8.35 (s, 1H), 7.71 (s, 1H), 7.68 (d, J = 8.4 Hz, 1H), 7.58 (d, J = 9.2 Hz, 1H), 7.40 (d, J = 8.8 Hz, 2H), 7.34- 7.20 (m, 3H), 7.29 (t, J_{HF} = 72 Hz, 1H), 6.55 (d, J = 8.8 Hz, 1H), 6.02 (tt, J_{HF} = 60 Hz, J = 3.6 Hz, 1H), 4.95 (s, 2H), 4.17 (s, 3H), 4.07 (td, J_{HF} = 14.4 Hz, J = 3.6 Hz, 2H). |
| | 7-(2,2-difluoroethoxy)-1-(4- | |
| | (difluoromethoxy)phenyl)-3-(2- | |
| | methyl-2H-indazol-5-yl)-3,4- | |
| | dihydropyrido[2,3-d]pyrimidin- | |
| | 2(1H)-one | |
| Example 146 | 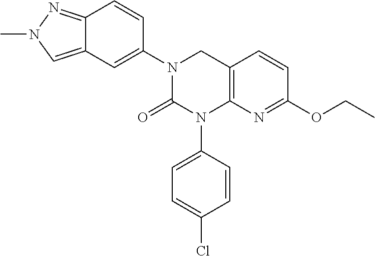 [SMILES: CCOc1ccc2c(n1)N(c1ccc(Cl)cc1)C(=O)N(c1ccc3nn(C)cc3c1)C2] | LC-MS (ESI): m/z 434.0 [M + H]+. 1H NMR (400 MHz, DMSO-d₆) δ: 8.35 (s, 1H), 7.69 (d, J = 1.6 Hz, 1H), 7.64-7.54 (m, 2H), 7.50 (d, J = 8.8 Hz, 2H), 7.38 (d, J = 8.8 Hz, 2H), 7.26 (dd, J = 9.2 Hz, 2.0 Hz, 1H), 6.42 (d, J = 8.4 Hz, 1H), 4.91 (s, 2H), 4.17 (s, 3H), 3.87 (q, J = 7.2 Hz, 2H), 1.07 (t, J = 7.2 Hz, 3H). |
| | 1-(4-chlorophenyl)-7-ethoxy-3-(2- | |
| | methyl-2H-indazol-5-yl)-3,4- | |
| | dihydropyrido[2,3-d]pyrimidin- | |
| | 2(1H)-one | |
| Example 147 | 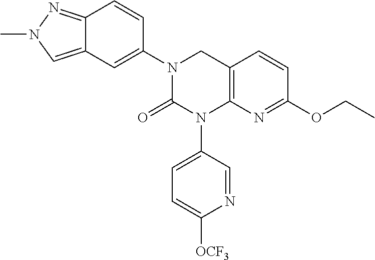 [SMILES: CCOc1ccc2c(n1)N(c1ccc(OC(F)(F)F)nc1)C(=O)N(c1ccc3nn(C)cc3c1)C2] | LC-MS (ESI): m/z 485.0 [M + H]+. 1H NMR (400 MHz, DMSO-d₆) δ: 8.40 (d, J = 2.4 Hz, 1H), 8.37 (s, 1H), 8.10 (dd, J = 8.8 Hz, 2.8 Hz, 1H), 7.73 (d, J = 2.0 Hz, 1H), 7.65 (d, J = 8.4 Hz, 1H), 7.60 (d, J = 8.8 Hz, 1H), 7.43 (d, J = 8.4 Hz, 1H), 7.31 (dd, J = 9.2 Hz, 2.0 Hz, 1H), 6.48 (d, J = 8.0 Hz, 1H), 4.95 (s, 2H), 4.18 (s, 3H), 3.88 (q, J = 7.2 Hz, 2H), 1.07 (t, J = 7.2 Hz, 3H). |
| | 7-ethoxy-3-(2-methyl-2H-indazol- | |
| | 5-yl)-1-(6- | |
| | (trifluoromethoxy)pyridin-3-yl)- | |
| | 3,4-dihydropyrido[2,3- | |
| | d]pyrimidin-2(1H)-one | |
| Example 148 | 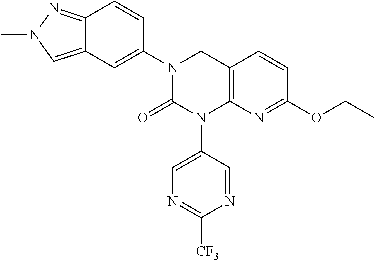 [SMILES: CCOc1ccc2c(n1)N(c1cnc(C(F)(F)F)nc1)C(=O)N(c1ccc3nn(C)cc3c1)C2] | LC-MS (ESI): m/z 470.0 [M + H]+. 1H NMR (400 MHz, DMSO-d₆) δ: 9.25 (s, 2H), 8.39 (s, 1H), 7.77 (s, 1H), 7.70 (d, J = 7.6 Hz, 1H), 7.62 (d, J = 8.4 Hz, 1H), 7.33 (d, J = 8.4 Hz, 1H), 6.56 (d, J = 7.6 Hz, 1H), 4.99 (s, 2H), 4.19 (s, 3H), 3.91 (q, J = 6.4 Hz, 2H), 1.10 (t, J = 6.4 Hz, 3H). |
| | 7-ethoxy-3-(2-methyl-2H-indazol- | |
| | 5-yl)-1-(2- | |
| | (trifluoromethyl)pyrimidin-5-yl)- | |
| | 3,4-dihydropyrido[2,3- | |
| | d]pyrimidin-2(1H)-one | |
| Example 149 | 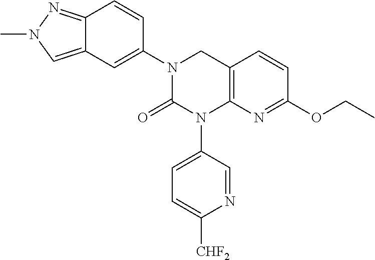 [SMILES: CCOc1ccc2c(n1)N(c1ccc(C(F)F)nc1)C(=O)N(c1ccc3nn(C)cc3c1)C2] | LC-MS (ESI): m/z 451.0 [M + H]+. 1H NMR (400 MHz, DMSO-d₆) δ: 8.70 (d, J = 2.0 Hz, 1H), 8.36 (s, 1H), 8.07 (dd, J = 8.4 Hz, 2.4 Hz, 1H), 7.82 (d, J = 8.4 Hz, 1H), 7.73 (d, J = 1.2 Hz, 1H), 7.65 (d, J = 8.4 Hz, 1H), 7.59 (d, J = 9.2 Hz, 1H), 7.31 (dd, J = 9.2 Hz, 2.0 Hz, 1H), 7.04 (t, J_{HF} = 55.2 Hz, 1H), 6.48 (d, J = 8.4 Hz, 1H), 4.95 (s, 2H), 4.17 (s, 3H), 3.85 (q, J = 6.8 Hz, 2H), 1.05 (t, J = 6.8 Hz, 3H). |
| | 1-(6-(difluoromethyl)pyridin-3- | |
| | yl)-7-ethoxy-3-(2-methyl-2H- | |
| | indazol-5-yl)-3,4- | |
| | dihydropyrido[2,3-d]pyrimidin- | |
| | 2(1H)-one | |
| Example 150 | 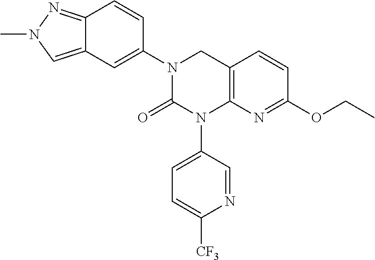 [SMILES: CCOc1ccc2c(n1)N(c1ccc(C(F)(F)F)nc1)C(=O)N(c1ccc3nn(C)cc3c1)C2] | LC-MS (ESI): m/z 469.0 [M + H]+. 1H NMR (400 MHz, DMSO-d₆) δ: 8.81 (d, J = 2.4 Hz, 1H), 8.37 (s, 1H), 8.18 (dd, J = 8.0 Hz, 2.0 Hz, 1H), 8.05 (d, J = 8.4 Hz, 1H), 7.74 (d, J = 1.2 Hz, 1H), 7.66 (d, J = 8.4 Hz, 1H), 7.60 (d, J = 9.2 Hz, 1H), 7.31 (dd, J = 9.2 Hz, 2.0 Hz, 1H), 6.50 (d, J = 8.4 Hz, 1H), 4.96 (s, 2H), 4.17 (s, 3H), 3.86 (q, J = 7.2 Hz, 2H), 1.05 (t, J = 7.2 Hz, 3H). |
| | 7-ethoxy-3-(2-methyl-2H-indazol- | |
| | 5-yl)-1-(2- | |
| | (trifluoromethyl)pyridin-3-yl)-3,4- | |
| | dihydropyrido[2,3-d]pyrimidin- | |
| | 2(1H)-one | |
| Example 151 | 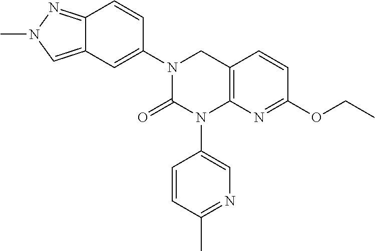 [SMILES: CCOc1ccc2c(n1)N(c1ccc(C)nc1)C(=O)N(c1ccc3nn(C)cc3c1)C2] | LC-MS (ESI): m/z 415.2 [M + H]+. 1H NMR (400 MHz, DMSO-d₆) δ: 8.51 (d, J = 5.6 Hz, 1H), 8.35 (s, 1H), 7.70 (d, J = 1.2 Hz, 1H), 7.62 (d, J = 8.4 Hz, 1H), 7.58 (d, J = 9.2 Hz, 1H), 7.30 (d, J = 1.2 Hz, 1H), 7.28 (dd, J = 9.2 Hz, 2.0 Hz, 1H), 7.23 (dd, J = 5.2 Hz, 1.6 Hz, 1H), 6.46 (d, J = 8.0 Hz, 1H), 4.92 (s, 2H), 4.17 (s, 3H), 3.87 (q, J = 7.2 Hz, 2H), 2.5 (s, 3H), 1.09 (t, J = 7.2 Hz, 3H). |
| | 7-ethoxy-3-(2-methyl-2H-indazol- | |
| | 5-yl)-1-(6-methylpyridin-3-yl)- | |
| | 3,4-dihydropyrido[2,3- | |
| | d]pyrimidin-2(1H)-one | |
| Example 152 | 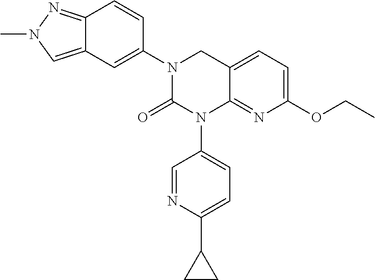 [SMILES: CCOc1ccc2c(n1)N(c1ccc(C3CC3)nc1)C(=O)N(c1ccc3nn(C)cc3c1)C2] | LC-MS (ESI): m/z 441.1 [M + H]+. 1H NMR (400 MHz, DMSO-d₆) δ 8.33-8.30 (m, 2H), 7.68 (s, 1H), 7.66-7.52 (m, 3H), 7.36 (d, J = 8.3 Hz, 1H), 7.26 (d, J = 9.6 Hz, 1H), 6.42 (d, J = 8.1 Hz, 1H), 4.90 (s, 2H), 4.15 (s, 3H), 3.85 (q, J = 6.9 Hz, 2H), 2.17-2.10 (m, 1H), 1.05 (t, J = 7.1 Hz, 3H), 1.01-0.91 (m, 4H). |
| | 1-(6-cyclopropylpyridin-3-yl)-7- | |
| | ethoxy-3-(2-methyl-2H-indazol-5- | |
| | yl)-3,4-dihydropyrido[2,3- | |
| | d]pyrimidin-2(1H)-one | |
| Example 153 | 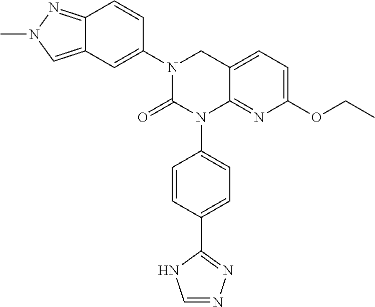 [SMILES: CCOc1ccc2c(n1)N(c1ccc(-c3nnc[nH]3)cc1)C(=O)N(c1ccc3nn(C)cc3c1)C2] | LC-MS (ESI): m/z 467.2 [M + H]+. 1H NMR (400 MHz, DMSO-d₆) δ 14.13 (s, 1H), 8.64 (s, 1H), 8.35 (s, 1H), 8.09 (d, J = 8.2 Hz, 2H), 7.71 (s, 1H), 7.61 (d, J = 8.2 Hz, 1H), 7.58 (d, J = 9.1 Hz, 1H), 7.52 (d, J = 7.3 Hz, 1H), 7.42 (d, J = 8.0 Hz, 1H), 7.29 (d, J = 9.3 Hz, 1H), 6.43 (d, J = 8.0 Hz, 1H), 4.93 (s, 2H), 4.17 (s, 3H), 3.85 (q, J = 7.0 Hz, 2H), 1.05 (t, J = 7.0 Hz, 3H). |
| | 1-(4-(4H-1,2,4-triazol-3- | |
| | yl)phenyl)-7-ethoxy-3-(2-methyl- | |
| | 2H-indazol-5-yl)-3,4- | |
| | dihydropyrido[2,3-d]pyrimidin- | |
| | 2(1H)-one | |
| Example 154 | 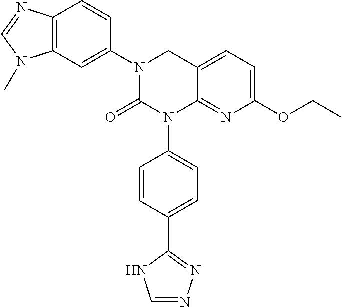 [SMILES: CCOc1ccc2c(n1)N(c1ccc(-c3nnc[nH]3)cc1)C(=O)N(c1ccc3ncn(C)c3c1)C2] | LC-MS (ESI): m/z 467.1 [M + H]+. 1H NMR (400 MHz, DMSO-d₆) δ 14.19 (s, 1H), 8.55 (s, 1H), 8.19 (s, 1H), 8.07 (d, J = 8.1 Hz, 2H), 7.66 (s, 1H), 7.64 (d, J = 8.8 Hz, 1H), 7.60 (d, J = 8.2 Hz, 1H), 7.44 (d, J = 8.1 Hz, 2H), 7.27 (d, J = 8.8 Hz, 1H), 6.42 (d, J = 8.2 Hz, 1H), 4.94 (s, 2H), 3.84 (q, J = 6.9 Hz, 2H), 3.82 (s, 3H), 1.03 (t, J = 6.9 Hz, 3H). |
| | 1-(4-(4H-1,2,4-triazol-3- | |
| | yl)phenyl)-7-ethoxy-3-(1-methyl- | |
| | 1H-benzo[d]imidazol-6-yl)-3,4- | |
| | dihydropyrido[2,3-d]pyrimidin- | |
| | 2(1H)-one | |
| Example 155 | 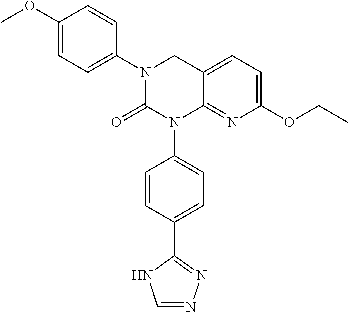 [SMILES: CCOc1ccc2c(n1)N(c1ccc(-c3nnc[nH]3)cc1)C(=O)N(c1ccc(OC)cc1)C2] | LC-MS (ESI): m/z 443.1 [M + H]+. 1H NMR (400 MHz, Chloroform-d) δ 8.07 (s, 1H), 7.90 (d, J = 8.1 Hz, 2H), 7.40-7.31 (m, 5H), 6.95 (d, J = 7.3 Hz, 2H), 6.36 (d, J = 7.9 Hz, 1H), 4.84 (s, 2H), 3.90-3.79 (m, 5H), 1.08 (t, J = 7.1 Hz, 3H). (triazole NH not observed) |
| | 1-(4-(4H-1,2,4-triazol-3- | |
| | yl)phenyl)-7-ethoxy-3-(4- | |
| | methoxyphenyl)-3,4- | |
| | dihydropyrido[2,3-d]pyrimidin- | |
| | 2(1H)-one | |
| Example 403 | 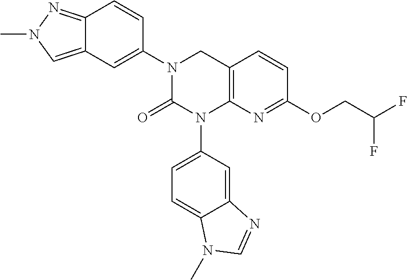 [SMILES: Cn1cc2cc(N3Cc4ccc(OCC(F)F)nc4N(c4ccc5c(c4)ncn5C)C3=O)ccc2n1] | LC-MS: m/z 490 (M + H)+. 1H NMR (400 MHz, DMSO-d₆) δ: 8.34 (s, 1H), 8.20 (s, 1H), 7.70 (d, J = 1.7 Hz, 1H), 7.67 (d, J = 8.1 Hz, 1H), 7.61 (s, 1H), 7.60(d J = 8.7 Hz, 1H), 7.56 (d, J = 9.2 Hz, 1H), 7.29 (dd, J = 9.2 Hz, 1.8 Hz, 1H), 7.23 (dd, J = 8.5 Hz, 1.5 Hz, 1H), 6.51 (d, J = 8.0 Hz, 1H), 5.91 (tt, J_{HF} = 55.5 Hz, J = 3.8 Hz, 1H), 4.96 (s, 2H), 4.16 (s, 3H), 3.95 (td, J_{HF} = 14.5 Hz, 3.8 Hz, 2H), 3.86 (s, 3H). |
| | 7-(2,2-difluoroethoxy)-1-(1- | |
| | methyl-1H-benzo[d]imidazol-5- | |
| | yl)-3-(2-methyl-2H-indazol-5-yl)- | |
| | 3,4-dihydropyrido[2,3- | |
| | d]pyrimidin-2(1H)-one | |
| Example 404 | 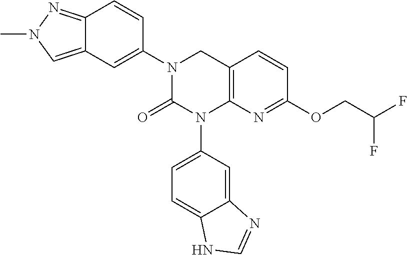 [SMILES: Cn1cc2cc(N3Cc4ccc(OCC(F)F)nc4N(c4ccc5[nH]cnc5c4)C3=O)ccc2n1] | LC-MS: m/z 476 (M + H)+. 1H NMR (400 MHz, DMSO-d₆) δ: 12.51 (s, 1H), 8.33 (s, 1H), 8.24 (s, 1H), 7.70 (s, 1H), 7.66 (d, J = 8.1 Hz, 1H), 7.56 (d, J = 9.2 Hz, 1H), 7.55 (s, 2H), 7.28 (d, J = 9.1 Hz, 1H), 7.14 (s, 1H), 6.51 (d, J = 8.0 Hz, 1H), 5.89 (t, J_{HF} = 55.4 Hz, 1H), 4.95 (s, 2H), 4.15 (s, 3H), 3.94 (td, J_{HF} = 14.5 Hz, J = 3.5 Hz, 2H). |
| | 1-(1H-benzo[d]imidazol-5-yl)-7- | |
| | (2,2-difluoroethoxy)-3-(2-methyl- | |
| | 2H-indazol-5-yl)-3,4- | |
| | dihydropyrido[2,3-d]pyrimidin- | |
| | 2(1H)-one | |
| Example 405 | 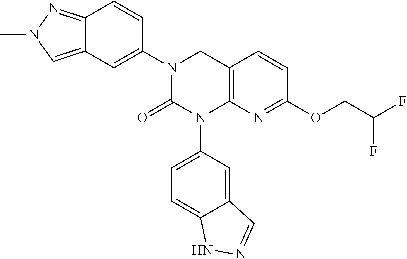 [SMILES: Cn1cc2cc(N3Cc4ccc(OCC(F)F)nc4N(c4ccc5[nH]ncc5c4)C3=O)ccc2n1] | LC-MS: m/z 476 (M + H)+. 1H NMR (400 MHz, DMSO-d₆) δ: 13.15 (s, 1H), 8.35 (s, 1H), 8.10 (s, 1H), 7.75 (s, 1H), 7.72 (s, 1H), 7.68 (d, J = 8.0 Hz, 1H), 7.60 (d, J = 3.5 Hz, 1H), 7.57 (d, J = 3.9 Hz, 1H), 7.31 (d, J = 4.8 Hz, 1H), 7.28 (s, 1H), 6.53 (d, J = 8.0 Hz, 1H), 5.93 (tt, J_{HF} = 55.3 Hz, J = 3.8 Hz, 1H), 4.97 (s, 2H), 4.17 (s, 3H), 3.96 (td, J_{HF} = 14.4, J = 3.8 Hz, 2H). |
| | 7-(2,2-difluoroethoxy)-1-(1H- | |
| | indazol-5-yl)-3-(2-methyl-2H- | |
| | indazol-5-yl)-3,4- | |
| | dihydropyrido[2,3-d]pyrimidin- | |
| | 2(1H)-one | |
| Example 406 | 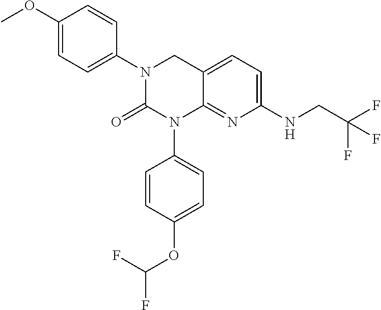 [SMILES: COc1ccc(N2Cc3ccc(NCC(F)(F)F)nc3N(c3ccc(OC(F)F)cc3)C2=O)cc1] | LC-MS: m/z 495 (M + H)+. 1H NMR (400 MHz, DMSO-d₆) δ: 7.35 (d, J = 8.2 Hz, 1H), 7.32 (d, J = 8.7 Hz, 2H), 7.30 (d, J = 8.6 Hz, 2H), 7.24 (t, J_{HF} = 7.2 Hz, 1H), 7.21 (d, J = 8.6 Hz, 2H), 7.06 (d, J = 3.6 Hz, 1H), 6.95 (d, J = 8.7 Hz, 2H), 6.23 (d, J = 8.1 Hz, 1H), 4.74 (s, 2H), 3.76 (s, 3H), 3.73-3.59 (m, 2H). |
| | 1-(4-(difluoromethoxy)phenyl)-3- | |
| | (4-methoxyphenyl)-7-((2,2,2- | |
| | trifluoroethyl)amino)-3,4- | |
| | dihydropyrido[2,3-d]pyrimidin- | |
| | 2(1H)-one | |
| Example 407 | 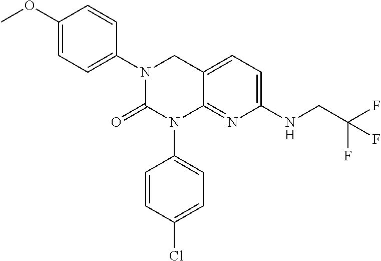 [SMILES: COc1ccc(N2Cc3ccc(NCC(F)(F)F)nc3N(c3ccc(Cl)cc3)C2=O)cc1] | LC-MS: m/z 463 (M + H)+. 1H NMR (400 MHz, DMSO-d₆) δ: 7.44 (d, J = 8.3 Hz, 2H), 7.34 (d, J = 8.2 Hz, 1H), 7.30 (d, J = 8.7 Hz, 2H), 7.26 (d, J = 8.4 Hz, 2H), 7.07 (t, J = 6.4 Hz, 1H), 6.94 (d, J = 8.7 Hz, 2H), 6.22 (d, J = 8.1 Hz, 1H), 4.72 (s, 2H), 3.75 (s, 3H), 3.71-3.59 (m, 2H). |
| | 1-(4-chlorophenyl)-3-(4- | |
| | methoxyphenyl)-7-((2,2,2- | |
| | trifluoroethyl)amino)-3,4- | |
| | dihydropyrido[2,3-d]pyrimidin- | |
| | 2(1H)-one | |
| Example 408 | 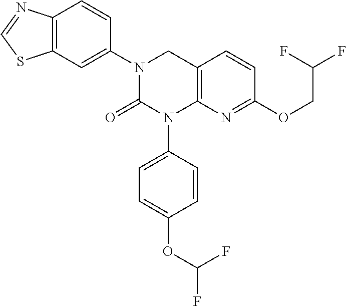 [SMILES: O=C1N(c2ccc3ncsc3c2)Cc2ccc(OCC(F)F)nc2N1c1ccc(OC(F)F)cc1] | LC-MS: m/z 505 (M + H)+. 1H NMR (400 MHz, DMSO-d₆) δ: 9.39 (s, 1H), 8.25 (s, 1H), 8.11 (d, J = 8.8 Hz, 1H), 7.69 (d, J = 8.4 Hz, 1H), 7.63 (d, J = 8.8 Hz, 1H), 7.44 (d, J = 8.4 Hz, 2H), 7.29 (d, J = 8.4 Hz, 2H), 7.27 (t, J_{HF} = 73.6 Hz, 1H), 6.57 (d, J = 8.0 Hz, 1H), 6.01 (tt, J_{HF} = 55.6 Hz, J = 3.2 Hz, 1H), 5.01 (s, 2H), 4.07 (td, J_{HF} = 14.4 Hz, J = 3.6 Hz, 2H). |
| | 3-(benzo[d]thiazol-6-yl)-7-(2,2- | |
| | difluoroethoxy)-1-(4- | |
| | (difluoromethoxy)phenyl)-3,4- | |
| | dihydropyrido[2,3-d]pyrimidin- | |
| | 2(1H)-one | |
| Example 409 | 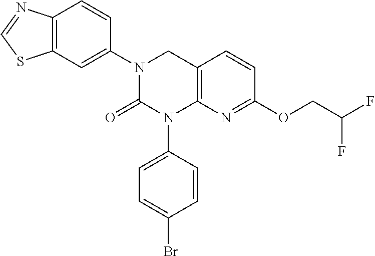 [SMILES: O=C1N(c2ccc3ncsc3c2)Cc2ccc(OCC(F)F)nc2N1c1ccc(Br)cc1] | LC-MS: m/z 518 (M + H)+. 1H NMR (400 MHz, DMSO-d₆) δ: 9.40 (s, 1H), 8.25 (s, 1H), 8.10 (d, J = 9.2 Hz, 1H), 7.72-7.61 (m, 4H), 7.37(d, J = 8.0 Hz, 2H), 6.58 (d, J = 8.0 Hz, 1H), 6.05 (t, J_{HF} = 15.6 Hz, J = 3.2 Hz, 1H), 5.01 (s, 2H), 4.09 (td, J_{HF} = 14.8 Hz, J = 3.2 Hz, 2H). |
| | 3-(benzo[d]thiazol-6-yl)-1-(4- | |
| | bromophenyl)-7-(2,2- | |
| | difluoroethoxy)-3,4- | |
| | dihydropyrido[2,3-d]pyrimidin- | |
| | 2(1H)-one | |
| Example 410 5-(4,4,5,5- tetramethyl- 1,3,2- dioxaborolan- 2-yl)-1-((2- (trimethylsilyl) ethoxy)meth- yl)-1H- indole-3- carbonitrile (Ref: WO2018215316) | 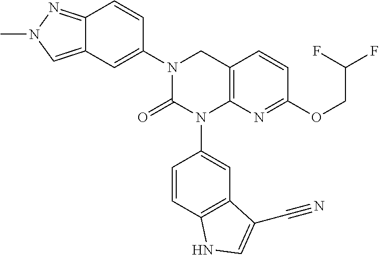 [SMILES: Cn1cc2cc(N3Cc4ccc(OCC(F)F)nc4N(c4ccc5[nH]cc(C#N)c5c4)C3=O)ccc2n1] | LC-MS: m/z 500 (M + H)+. 1H NMR (400 MHz, DMSO-d₆) δ: 12.28 (s, 1H), 8.34 (s, 1H), 8.28 (s, 1H), 7.73-7.54 (m, 5H), 7.28 (dd, J = 8.9 Hz, 1.9 Hz, 2H), 6.53 (d, J = 8.0 Hz, 1H), 5.93 (tt, J_{HF} = 55.6 Hz, J = 3.8 Hz, 1H), 4.96 (s, 2H), 4.16 (s, 3H), 3.97 (td, J_{HF} = 14.5, J = 3.8 Hz, 2H). |
| | 5-(7-(2,2-difluoroethoxy)-3-(2- | |
| | methyl-2H-indazol-5-yl)-2-oxo- | |
| | 3,4-dihydropyrido[2,3- | |
| | d]pyrimidin-1(2H)-yl)-1H-indole- | |
| | 3-carbonitrile | |
| Example 411 | 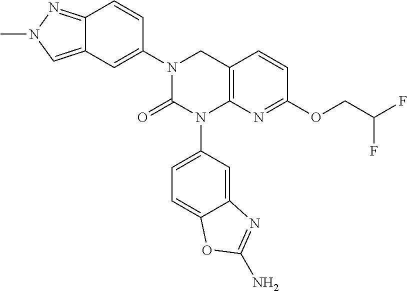 [SMILES: Cn1cc2cc(N3Cc4ccc(OCC(F)F)nc4N(c4ccc5oc(N)nc5c4)C3=O)ccc2n1] | LC-MS: m/z 492 (M + H)+. 1H NMR (400 MHz, DMSO-d₆) δ: 8.33 (s, 1H), 7.69 (s, 1H), 7.65 (d, J = 8.1 Hz, 1H), 7.57 (d, J = 9.1 Hz, 1H), 7.43 (s, 2H), 7.37 (d, J = 8.4 Hz, 1H), 7.28 (dd, J = 9.2 Hz, 1.7 Hz, 1H), 7.16 (d, J = 1.7 Hz, 1H), 6.92 (dd, J = 8.4 Hz, 1.8 Hz, 1H), 6.51 (d, J = 8.0 Hz, 1H), 6.02 (tt, J_{HF} = 55.6 Hz, J = 3.6 Hz, 1H), 4.93 (s, 2H), 4.16 (s, 3H), 4.01 (td, J_{HF} = 14.4 Hz, J = 3.6 Hz, 2H). |
| | 1-(2-aminobenzo[d]oxazol-5-yl)- | |
| | 7-(2,2-difluoroethoxy)-3-(2- | |
| | methyl-2H-indazol-5-yl)-3,4- | |
| | dihydropyrido[2,3-d]pyrimidin- | |
| | 2(1H)-one | |
| Example 412 | 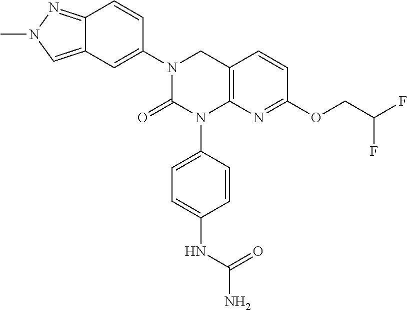 [SMILES: Cn1cc2cc(N3Cc4ccc(OCC(F)F)nc4N(c4ccc(NC(N)=O)cc4)C3=O)ccc2n1] | LC-MS: m/z 494 (M + H)+. 1H NMR (400 MHz, DMSO-d₆) δ: 8.81 (s, 1H), 8.35 (s, 1H), 7.69 (d, J = 1.4 Hz, 1H), 7.66 (d, J = 8.1 Hz, 1H), 7.57 (d, J = 9.2 Hz, 1H), 7.48 (d, J = 8.8 Hz, 2H), 7.26 (dd, J = 9.2 Hz, 2.0 Hz, 1H), 7.17 (d, J = 8.8 Hz, 2H), 6.52 (d, J = 8.0 Hz, 1H), 6.12 (tt, J_{HF} = 55.4 Hz, J = 3.8 Hz, 1H), 5.91 (s, 2H), 4.92 (s, 2H), 4.17 (s, 3H), 4.09 (td, J_{HF} = 14.5 Hz, J = 3.8 Hz, 2H). |
| | 1-(4-(7-(2,2-difluoroethoxy)-3-(2- | |
| | methyl-2H-indazol-5-yl)-2-oxo- | |
| | 3,4-dihydropyrido[2,3- | |
| | d]pyrimidin-1(2H)-yl)phenyl)urea | |

The procedure set forth above for General Procedure II (Method B) was used to synthesize the following compounds by using appropriate starting materials:

| Cpd No. | Structure | Characterization |
|---|---|---|
| Example 157 | 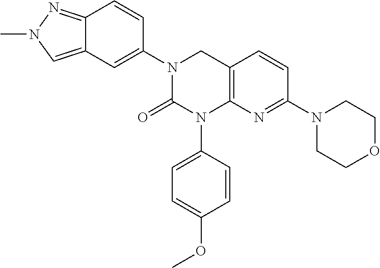 [SMILES: COc1ccc(N2C(=O)N(c3ccc4nn(C)cc4c3)Cc3ccc(N4CCOCC4)nc32)cc1] | LC-MS: m/z 471.2 (M + H)+. 1H NMR (400 MHz, DMSO-d₆) δ: 8.33 (s, 1H), 7.66 (d, J = 1.2 Hz, 1H), 7.55 (d, J = 9.2 Hz, 1H), 7.45 (d, J = 8.4 Hz, 1H), 7.25 (dd, J = 9.2 Hz, 2.0 Hz, 1H), 7.19 (d, J = 8.8 Hz, 2H), 6.96 (d, J = 8.8 Hz, 2H), 6.43 (d, J = 8.4 Hz, 1H), 4.83 (s, 2H), 4.16 (s, 3H), 3.78 (s, 3H), 3.57-3.52 (m, 4H), 3.16-3.10 (m, 4H). |
| | 1-(4-methoxyphenyl)-3-(2- | |
| | methyl-2H-indazol-5-yl)-7- | |
| | morpholino-3,4- | |
| | dihydropyrido[2,3- | |
| | d]pyrimidin-2(1H)-one | |
| Example 158 | 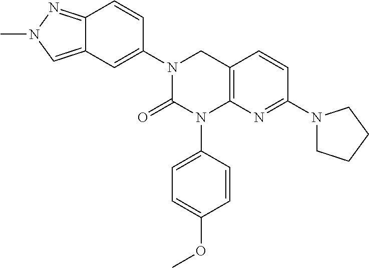 [SMILES: COc1ccc(N2C(=O)N(c3ccc4nn(C)cc4c3)Cc3ccc(N4CCCC4)nc32)cc1] | LC-MS: m/z 455.2 (M + H)+. 1H NMR (400 MHz, DMSO-d₆) δ: 8.32 (s, 1H), 7.65 (d, J = 1.6 Hz, 1H), 7.55 (d, J = 9.2 Hz, 1H), 7.36 (d, J = 8.4 Hz, 1H), 7.25 (dd, J = 9.2 Hz, 2.0 Hz, 1H), 7.19 (d, J = 8.8 Hz, 2H), 6.94 (d, J = 9.2 Hz, 2H), 6.04 (d, J = 8.0 Hz, 1H), 4.80 (s, 2H), 4.16 (s, 3H), 3.78 (s, 3H), 3.12-3.02 (m, 4H), 1.85-1.75 (m, 4H). |
| | 1-(4-methoxyphenyl)-3-(2- | |
| | methyl-2H-indazol-5-yl)-7- | |
| | (pyrrolidin-1-yl)-3,4- | |
| | dihydropyrido[2,3- | |
| | d]pyrimidin-2(1H)-one | |
| Example 159 | 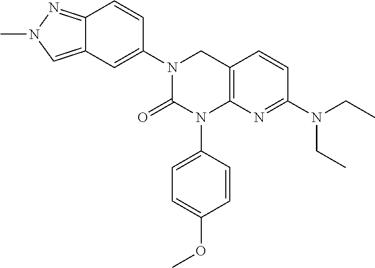 [SMILES: CCN(CC)c1ccc2c(n1)N(c1ccc(OC)cc1)C(=O)N(c1ccc3nn(C)cc3c1)C2] | LC-MS: m/z 457.2 (M + H)+. 1H NMR (400 MHz, DMSO-d₆) δ: 8.33 (s, 1H), 7.66 (d, J = 1.6 Hz, 1H), 7.55 (d, J = 8.8 Hz, 1H), 7.33 (d, J = 8.4 Hz, 1H), 7.25 (dd, J = 9.2 Hz, 2.0 Hz, 1H), 7.18 (d, J = 8.8 Hz, 2H), 6.95 (d, J = 9.2 Hz, 2H), 6.17 (d, J = 8.4 Hz, 1H), 4.79 (s, 2H), 4.16 (s, 3H), 3.78 (s, 3H), 3.16 (q, J = 6.8 Hz, 4H), 0.86 (q, J = 6.8 Hz, 6H). |
| | 7-(diethylamino)-1-(4- | |
| | methoxyphenyl)-3-(2-methyl- | |
| | 2H-indazol-5-yl)-3,4- | |
| | dihydropyrido[2,3- | |
| | d]pyrimidin-2(1H)-one | |
| Example 160 | 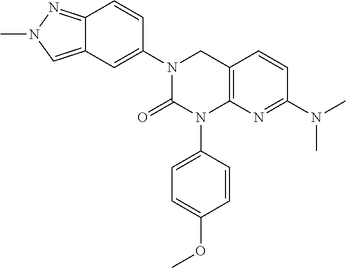 | LC-MS: m/z 429.2 (M + H)+. 1H NMR (400 MHz, DMSO-d₆) δ: 8.33 (s, 1H), 7.66 (d, J = 1.2 Hz, 1H), 7.55 (d, J = 9.2 Hz, 1H), 7.38 (d, J = 8.4 Hz, 1H), 7.25 (dd, J = 9.2 Hz, 2.0 Hz, 1H), 7.19 (d, J = 9.2 Hz, 2H), 6.96 (d, J = 8.8 Hz, 2H), 6.23 (d, J = 8.4 Hz, 1H), 4.81 (s, 2H), 4.16 (s, 3H), 3.78 (s, 3H), 2.74 (s, 6H). |
| | 7-(dimethylamino)-1-(4- | |
| | methoxyphenyl)-3-(2-methyl- | |
| | 2H-indazol-5-yl)-3,4- | |
| | dihydropyrido[2,3- | |
| | d]pyrimidin-2(1H)-one | |
| Example 161 | 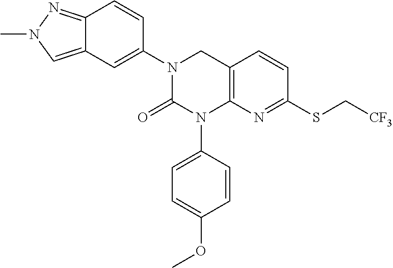 [SMILES: COc1ccc(N2C(=O)N(c3ccc4nn(C)cc4c3)Cc3ccc(SCC(F)(F)F)nc32)cc1] | LC-MS: m/z 500.1 (M + H)+. 1H NMR (400 MHz, DMSO-d₆) δ: 8.35 (s, 1H), 7.70 (d, J = 1.6 Hz, 1H), 7.59 (d, J = 8.0 Hz, 1H), 7.58 (d, J = 9.2 Hz, 1H), 7.29 (dd, J = 9.2 Hz, 2.0 Hz, 1H), 7.21 (d, J = 8.8 Hz, 2H), 7.04 (d, J = 7.6 Hz, 1H), 7.00 (d, J = 9.2 Hz, 2H), 4.97 (s, 2H), 4.17 (s, 3H), 3.78 (s, 3H), 3.70 (q, J = 10.4 Hz, 2H). |
| | 1-(4-methoxyphenyl)-3-(2- | |
| | methyl-2H-indazol-5-yl)-7- | |
| | ((2,2,2-trifluoroethyl)thio)-3,4- | |
| | dihydropyrido[2,3- | |
| | d]pyrimidin-2(1H)-one | |
| Example 162 | 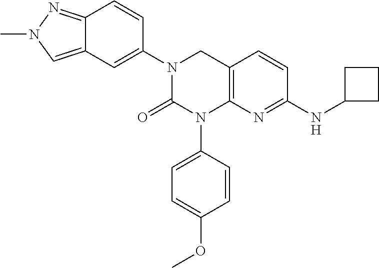 [SMILES: COc1ccc(N2C(=O)N(c3ccc4nn(C)cc4c3)Cc3ccc(NC4CCC4)nc32)cc1] | LC-MS: m/z 455.2 (M + H)+. 1H NMR (400 MHz, DMSO-d₆) δ: 8.32 (s, 1H), 7.64 (d, J = 1.2 Hz, 1H), 7.54 (d, J = 9.2 Hz, 1H), 7.28- 7.22 (m, 2H), 7.17 (d, J = 8.8 Hz, 1H), 6.96 (d, J = 8.8 Hz, 2H), 6.54 (d, J = 6.8 Hz, 1H), 6.02 (d, J = 8.0 Hz, 1H), 4.76 (s, 2H), 4.16 (s, 3H), 3.79 (s, 3H), 3.76-3.63 (m, 2H), 2.00-1.88 (m, 2H), 1.78-1.65 (m, 2H), 1.59-138 (m, 2H). |
| | 7-(cyclobutylamino)-1-(4- | |
| | methoxyphenyl)-3-(2-methyl- | |
| | 2H-indazol-5-yl)-3,4- | |
| | dihydropyrido[2,3- | |
| | d]pyrimidin-2(1H)-one | |
| Example 163 | 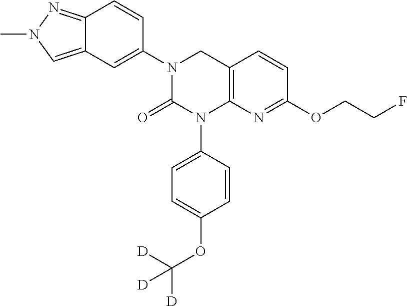 [SMILES: [2H]C([2H])([2H])Oc1ccc(N2C(=O)N(c3ccc4nn(C)cc4c3)Cc3ccc(OCCF)nc32)cc1] | LC-MS: m/z 451.2 (M + H)+. 1H NMR (400 MHz, DMSO-d₆) δ: 8.34 (s, 1H), 7.68 (d, J = 1.2 Hz, 1H), 7.62 (d, J = 8.4 Hz, 1H), 7.57 (d, J = 9.2 Hz, 1H), 7.29-7.21 (m, 3H), 6.98 (d, J = 8.8 Hz, 2H), 6.48 (d, J = 7.6 Hz, 1H), 4.91 (s, 2H), 4.54 (t, J = 4.0 Hz, 1H), 4.42 (t, J = 4.0 Hz, 1H), 4.17 (s, 3H), 4.08 (t, J = 4.0 Hz, 1H), 4.01 (t, J = 4.0 Hz, 1H). |
| | 7-(2-fluoroethoxy)-1-(4- | |
| | methoxy-d3-phenyl)-3-(2- | |
| | methyl-2H-indazol-5-yl)-3,4- | |
| | dihydropyrido[2,3- | |
| | d]pyrimidin-2(1H)-one | |
| Example 164 | 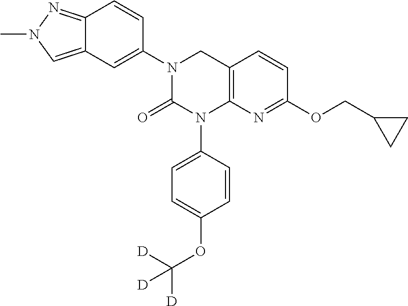 [SMILES: [2H]C([2H])([2H])Oc1ccc(N2C(=O)N(c3ccc4nn(C)cc4c3)Cc3ccc(OCC4CC4)nc32)cc1] | LC-MS: m/z 459.2 (M + H)+. 1H NMR (400 MHz, DMSO-d₆) δ: 8.34 (s, 1H), 7.68 (d, J = 1.6 Hz, 1H), 7.59-7.54 (m, 2H), 7.27 (dd, J = 9.2 Hz, 2.0 Hz, 1H), 7.21 (d, J = 8.8 Hz, 2H), 6.97 (d, J = 8.8 Hz, 2H), 6.40 (d, J = 8.0 Hz, 1H), 4.90 (s, 2H), 4.17 (s, 3H), 3.69 (d, J = 7.2 Hz, 2H), 1.03-0.93 (m, 1H), 0.40- 0.33 (m, 2H), 0.07-0.01 (m, 2H). |
| | 7-(cyclopropylmethoxy)-1-(4- | |
| | methoxy-d3-phenyl)-3-(2- | |
| | methyl-2H-indazol-5-yl)-3,4- | |
| | dihydropyrido[2,3- | |
| | d]pyrimidin-2(1H)-one | |
| Example 165 | 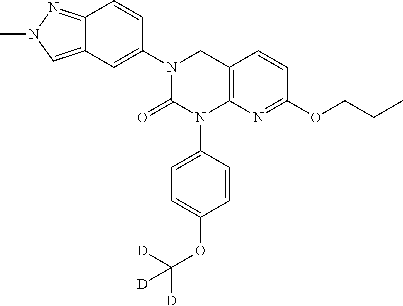 [SMILES: [2H]C([2H])([2H])Oc1ccc(N2C(=O)N(c3ccc4nn(C)cc4c3)Cc3ccc(OCCC)nc32)cc1] | LC-MS: m/z 447.2 (M + H)+. 1H NMR (400 MHz, DMSO-d₆) δ: 8.34 (s, 1H), 7.68 (d, J = 1.2 Hz, 1H), 7.59-7.54 (m, 2H), 7.27 (dd, J = 9.2 Hz, 2.0 Hz, 1H), 7.23 (d, J = 8.8 Hz, 2H), 6.98 (d, J = 8.8 Hz, 2H), 6.39 (d, J = 8.0 Hz, 1H), 4.90 (s, 2H), 4.17 (s, 3H), 3.78 (t, J = 7.2 Hz, 2H), 1.45 (q, J = 7.2 Hz, 2H), 0.72 (t, J = 7.2 Hz, 3H). |
| | 1-(4-methoxy-d3-phenyl)-3-(2- | |
| | methyl-2H-indazol-5-yl)-7- | |
| | propoxy-3,4- | |
| | dihydropyrido[2,3- | |
| | d]pyrimidin-2(1H)-one | |
| Example 166 | 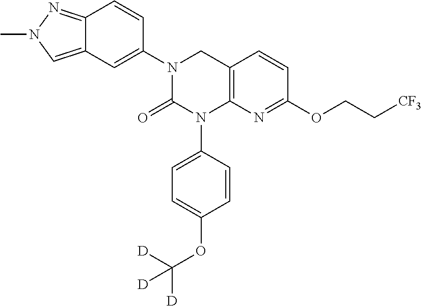 | LC-MS: m/z 447.2 (M + H)+. 1H NMR (400 MHz, DMSO-d₆) δ: 8.34 (s, 1H), 7.69 (d, J = 1.6 Hz, 1H), 7.62 (d, J = 8.0 Hz, 1H), 7.58 (d, J = 8.8 Hz, 1H), 7.29-7.22 (m, 3H), 6.98 (d, J = 8.8 Hz, 2H), 6.44 (d, J = 8.0 Hz, 1H), 4.92 (s, 2H), 4.17 (s, 3H), 4.04 (t, J = 6.4 Hz, 2H), 2.52-2.39 (m, 2H). |
| | 1-(4-methoxy-d3-phenyl)-3-(2- | |
| | methyl-2H-indazol-5-yl)-7- | |
| | (3,3,3-trifluoropropoxy)-3,4- | |
| | dihydropyrido[2,3- | |
| | d]pyrimidin-2(1H)-one | |
| Example 167 | 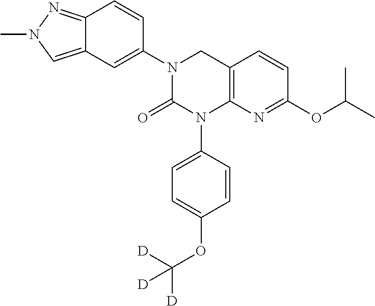 [SMILES: [2H]C([2H])([2H])Oc1ccc(N2C(=O)N(c3ccc4nn(C)cc4c3)Cc3ccc(OC(C)C)nc32)cc1] | LC-MS: m/z 447.2 (M + H)+. 1H NMR (400 MHz, DMSO-d₆) δ: 8.34 (s, 1H), 7.68 (d, J = 1.6 Hz, 1H), 7.58-7.53 (m, 2H), 7.27 (dd, J = 8.8 Hz, 2.0 Hz, 1H), 7.23 (d, J = 8.8 Hz, 2H), 6.98 (d, J = 8.8 Hz, 2H), 6.34 (d, J = 8.0 Hz, 1H), 4.89 (s, 2H), 4.52 (hept, J = 6.4 Hz, 1H), 4.17 (s, 3H), 1.05 (d, J = 6.4 Hz, 6H). |
| | 7-isopropoxy-1-(4-methoxy- | |
| | d3-phenyl)-3-(2-methyl-2H- | |
| | indazol-5-yl)-3,4- | |
| | dihydropyrido[2,3- | |
| | d]pyrimidin-2(1H)-one | |
| Example 168 | 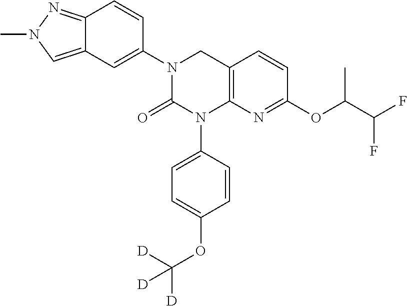 [SMILES: [2H]C([2H])([2H])Oc1ccc(N2C(=O)N(c3ccc4nn(C)cc4c3)Cc3ccc(OC(C)C(F)F)nc32)cc1] | LC-MS: m/z 483.2 (M + H)+. 1H NMR (400 MHz, DMSO-d₆) δ: 8.35 (s, 1H), 7.69 (s, 1H), 7.64 (d, J = 8.0 Hz, 1H), 7.57 (d, J = 9.2 Hz, 1H), 7.32-7.25 (m, 3H), 7.24 (t, J_{HF} = 74.0 Hz, 1H), 6.99 (d, J = 8.4 Hz, 1H), 6.48 (d, J = 8.4 Hz, 1H), 5.98 (tt, J_{HF} = 55.6 Hz, 4.0 Hz, 1H), 4.92 (s, 2H), 4.65-4.51 (m, 1H), 4.17 (s, 3H), 1.07 (d, J = 6.4 Hz, 3H). |
| | 7-((1,1-difluoropropan-2- | |
| | yl)oxy)-1-(4-methoxy-d3- | |
| | phenyl)-3-(2-methyl-2H- | |
| | indazol-5-yl)-3,4- | |
| | dihydropyrido[2,3- | |
| | d]pyrimidin-2(1H)-one | |
| Example 169 | 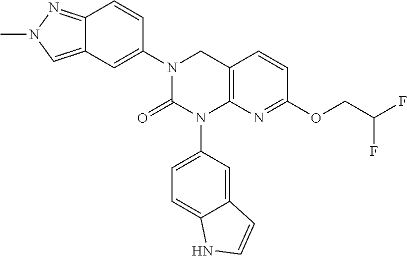 [SMILES: Cn1cc2cc(N3Cc4ccc(OCC(F)F)nc4N(c4ccc5[nH]ccc5c4)C3=O)ccc2n1] | LC-MS: m/z 475.2 (M + H)+. 1H NMR (400 MHz, DMSO-d₆) δ: 11.15 (s, 1H), 8.35 (s, 1H), 7.70 (d, J = 1.6 Hz, 1H), 7.66 (d, J = 8.0 Hz, 1H), 7.57 (d, J = 9.2 Hz, 1H), 7.49 (d, J = 1.6 Hz, 1H), 7.43 (d, J = 8.8 Hz, 1H), 7.37 (t, J = 6.8 Hz, 1H), 7.28 (dd, J = 9.2 Hz, 2.0 Hz, 1H), 7.03 (dd, J = 8.4 Hz, 2.0 Hz, 1H), 6.51 (d, J = 8.0 Hz, 1H), 6.46-6.43 (m, 1H), 5.92 (tt, J_{HF} = 55.2 Hz, 4.0 Hz, 1H), 4.96 (s, 2H), 4.17 (s, 3H), 3.96 (td, J_{HF} = 14.4 Hz, 4.0 Hz, 2H). |
| | 7-(2,2-difluoroethoxy)-1-(1H- | |
| | indol-5-yl)-3-(2-methyl-2H- | |
| | indazol-5-yl)-3,4- | |
| | dihydropyrido[2,3- | |
| | d]pyrimidin-2(1H)-one | |
| Example 170 | 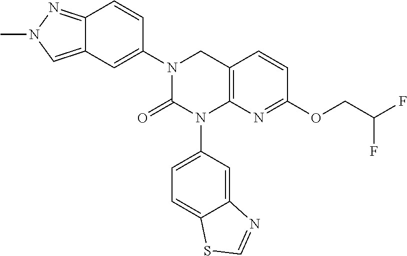 [SMILES: Cn1cc2cc(N3Cc4ccc(OCC(F)F)nc4N(c4ccc5scnc5c4)C3=O)ccc2n1] | LC-MS: m/z 493.1 (M + H)+. 1H NMR (400 MHz, DMSO-d₆) δ: 9.44 (s, 1H), 8.36 (s, 1H), 8.24 (d, J = 8.4 Hz, 1H), 8.11 (d, J = 1.6 Hz, 1H), 7.73 (d, J = 1.6 Hz, 1H), 7.71 (d, J = 8.4 Hz, 1H), 7.59 (d, J = 8.8 Hz, 1H), 7.50 (dd, J = 8.8 Hz, 2.0 Hz, 1H), 7.32 (dd, J = 9.2 Hz, 2.0 Hz, 1H), 6.56 (d, J = 8.4 Hz, 1H), 5.93 (tt, J_{HF} = 55.6 Hz, 4.0 Hz, 1H), 4.99 (s, 2H), 4.17 (s, 3H), 3.97 (td, J_{HF} = 14.4 Hz, 4.0 Hz, 2H). |
| | 1-(benzo[d]thiazol-5-yl)-7- | |
| | (2,2-difluoroethoxy)-3-(2- | |
| | methyl-2H-indazol-5-yl)-3,4- | |
| | dihydropyrido[2,3- | |
| | d]pyrimidin-2(1H)-one | |
| Example 171 | 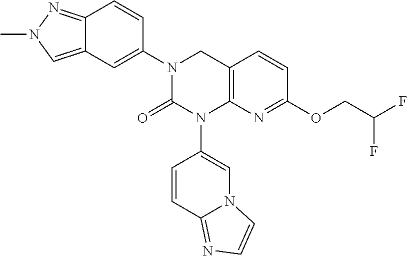 [SMILES: Cn1cc2cc(N3Cc4ccc(OCC(F)F)nc4N(c4ccc5nccn5c4)C3=O)ccc2n1] | LC-MS: m/z 476.2 (M + H)+. 1H NMR (400 MHz, DMSO-d₆) δ: 8.73 (s, 1H), 8.36 (s, 1H), 7.98 (s, 1H), 7.73-7.69 (m, 2H), 7.62-7.56 (m, 3H), 7.32-7.24 (m, 2H), 6.60 (d, J = 8.0 Hz, 1H), 6.10 (tt, J_{HF} = 55.2 Hz, 4.0 Hz, 1H), 4.97 (s, 2H), 4.17 (s, 3H), 4.10 (td, J_{HF} = 14.4 Hz, 4.0 Hz, 2H). |
| | 7-(2,2-difluoroethoxy)-1- | |
| | (imidazo[1,2-a]pyridin-6-yl)-3- | |
| | (2-methyl-2H-indazol-5-yl)- | |
| | 3,4-dihydropyrido[2,3- | |
| | d]pyrimidin-2(1H)-one | |
| Example 172 | 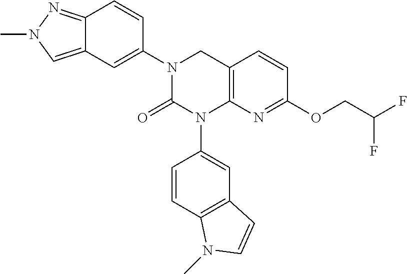 [SMILES: Cn1cc2cc(N3Cc4ccc(OCC(F)F)nc4N(c4ccc5c(ccn5C)c4)C3=O)ccc2n1] | LC-MS: m/z 489.2 (M + H)+. 1H NMR (400 MHz, DMSO-d₆) δ: 8.34 (s, 1H), 7.70 (d, J = 1.2 Hz, 1H), 7.66 (d, J = 9.2 Hz, 1H), 7.57 (d, J = 9.2 Hz, 1H), 7.50 (d, J = 1.6 Hz, 1H), 7.47 (d, J = 8.8 Hz, 1H), 7.35 (d, J = 3.2 Hz, 1H), 7.28 (dd, J = 9.2 Hz, 2.0 Hz, 1H), 7.09 (dd, J = 8.4 Hz, 1.6 Hz, 1H), 6.51 (d, J = 8.0 Hz, 1H), 6.45 (d, J = 2.8 Hz, 1H), 5.93 (tt, J_{HF} = 55.6 Hz, 4.0 Hz, 1H), 4.95 (s, 2H), 4.17 (s, 3H), 3.96 (td, J_{HF} = 14.4 Hz, 4.0 Hz, 2H), 3.82 (s, 3H). |
| | 7-(2,2-difluoroethoxy)-1-(1- | |
| | methyl-1H-indol-5-yl)-3-(2- | |
| | methyl-2H-indazol-5-yl)-3,4- | |
| | dihydropyrido[2,3- | |
| | d]pyrimidin-2(1H)-one | |
| Example 173 | 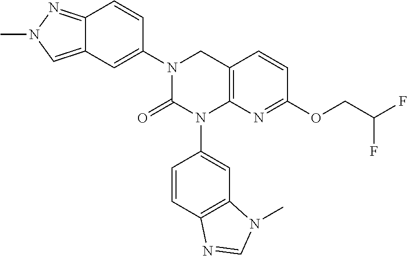 [SMILES: Cn1cc2cc(N3Cc4ccc(OCC(F)F)nc4N(c4ccc5ncn(C)c5c4)C3=O)ccc2n1] | LC-MS: m/z 490.2 (M + H)+. 1H NMR (400 MHz, DMSO-d₆) δ: 8.35 (s, 1H), 8.22 (s, 1H), 7.72 (d, J = 2.0 Hz, 1H), 7.70 (d, J = 3.2 Hz, 1H), 7.68 (d, J = 3.6 Hz, 1H), 7.63 (d, J = 1.6 Hz, 1H), 7.58 (d, J = 9.2 Hz, 1H), 7.29 (dd, J = 9.2 Hz, 2.0 Hz, 1H), 7.18 (dd, J = 8.8 Hz, 1.6 Hz, 1H), 6.54 (d, J = 7.6 Hz, 1H), 5.95 (tt, J_{HF} = 55.6 Hz, 4.0 Hz, 1H), 4.97 (s, 2H), 4.17 (s, 3H), 3.97 (td, J_{HF} = 14.4 Hz, 4.0 Hz, 2H), 3.84 (s, 3H). |
| | 7-(2,2-difluoroethoxy)-1-(1- | |
| | methyl-1H-benzo[d]imidazol- | |
| | 6-yl)-3-(2-methyl-2H-indazol- | |
| | 5-yl)-3,4-dihydropyrido[2,3- | |
| | d]pyrimidin-2(1H)-one | |
| Example 174 | 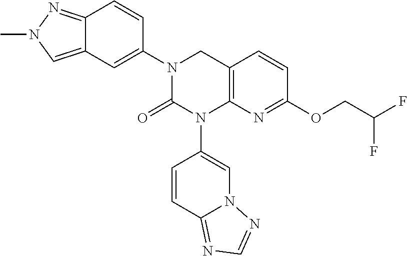 [SMILES: Cn1cc2cc(N3Cc4ccc(OCC(F)F)nc4N(c4ccc5ncnn5c4)C3=O)ccc2n1] | LC-MS: m/z 477.2 (M + H)+. 1H NMR (400 MHz, DMSO-d₆) δ: 9.21 (s, 1H), 8.56 (s, 1H), 8.37 (s, 1H), 7.91 (d, J = 9.6 Hz, 1H), 7.79 (dd, J = 9.6 Hz, 1.6 Hz, 1H), 7.75- 7.71 (m, 2H), 7.60 (d, J = 8.8 Hz, 1H), 7.32 (dd, J = 9.2 Hz, 1.2 Hz, 1H), 6.62 (d, J = 8.0 Hz, 1H), 6.08 (tt, J_{HF} = 55.6 Hz, 4.0 Hz, 1H), 4.99 (s, 2H), 4.17 (s, 3H), 4.08 (td, J_{HF} = 14.4 Hz, 4.0 Hz, 2H). |
| | 1-([1,2,4]triazolo[1,5- | |
| | a]pyridin-6-yl)-7-(2,2- | |
| | difluoroethoxy)-3-(2-methyl- | |
| | 2H-indazol-5-yl)-3,4- | |
| | dihydropyrido[2,3- | |
| | d]pyrimidin-2(1H)-one | |
| Example 175 | 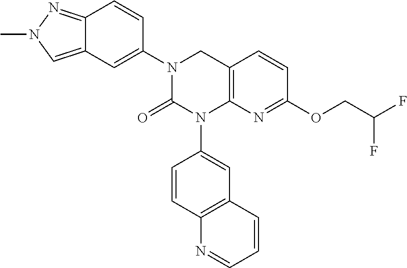 [SMILES: Cn1cc2cc(N3Cc4ccc(OCC(F)F)nc4N(c4ccc5ncccc5c4)C3=O)ccc2n1] | LC-MS: m/z 487.2 (M + H)+. 1H NMR (400 MHz, DMSO-d₆) δ: 9.00 (d, J = 3.2 Hz, 1H), 8.53 (d, J = 3.6 Hz, 1H), 8.37 (s, 1H), 8.14- 8.07 (m, 2H), 7.83 (dd, J = 9.2 Hz, 1.6 Hz, 1H), 7.75-7.70 (m, 2H), 7.65 (dd, J = 8.0 Hz, 4.0 Hz, 1H), 7.59 (d, J = 9.2 Hz, 1H), 7.32 (dd, J = 8.8 Hz, 1.6 Hz, 1H), 6.59 (d, J = 8.0 Hz, 1H), 5.96 (tt, J_{HF} = 55.6 Hz, 4.0 Hz, 1H), 5.01 (s, 2H), 4.17 (s, 3H), 3.96 (td, J_{HF} = 14.4 Hz, 4.0 Hz, 2H). |
| | 7-(2,2-difluoroethoxy)-3-(2- | |
| | methyl-2H-indazol-5-yl)-1- | |
| | (quinolin-6-yl)-3,4- | |
| | dihydropyrido[2,3- | |
| | d]pyrimidin-2(1H)-one | |
| Example 176 | 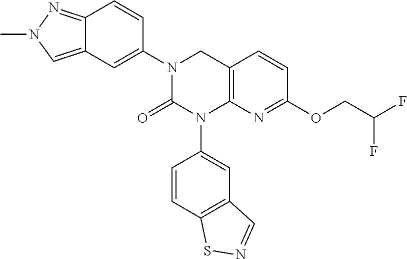 [SMILES: Cn1cc2cc(N3Cc4ccc(OCC(F)F)nc4N(c4ccc5sncc5c4)C3=O)ccc2n1] | LC-MS: m/z 493.2 (M + H)+. 1H NMR (400 MHz, DMSO-d₆) δ: 9.14 (s, 1H), 8.36 (s, 1H), 8.29 (d, J = 8.4 Hz, 1H), 8.24 (s, 1H), 7.74- 7.69 (m, 2H), 7.62 (dd, J = 8.8 Hz, 1.2 Hz, 1H), 7.59 (d, J = 9.2 Hz, 1H), 7.31 (dd, J = 9.2 Hz, 1.6 Hz, 1H), 6.57 (d, J = 8.0 Hz, 1H), 5.96 (tt, J_{HF} = 55.2 Hz, 4.0 Hz, 1H), 5.00 (s, 2H), 4.17 (s, 3H), 3.97 (td, J_{HF} = 14.4 Hz, 4.0 Hz, 2H). |
| | 1-(benzo[d]isothiazol-5-yl)-7- | |
| | (2,2-difluoroethoxy)-3-(2- | |
| | methyl-2H-indazol-5-yl)-3,4- | |
| | dihydropyrido[2,3- | |
| | d]pyrimidin-2(1H)-one | |
| Example 177 | 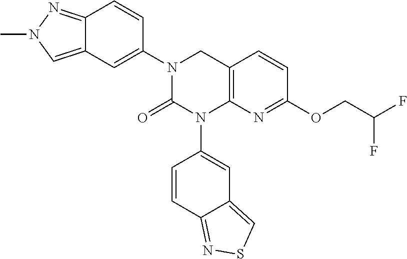 [SMILES: Cn1cc2cc(N3Cc4ccc(OCC(F)F)nc4N(c4ccc5nscc5c4)C3=O)ccc2n1] | LC-MS: m/z 493.2 (M + H)+. 1H NMR (400 MHz, DMSO-d₆) δ: 9.78 (s, 1H), 8.36 (s, 1H), 7.94 (s, 1H), 7.82 (d, J = 9.2 Hz, 1H), 7.74- 7.68 (m, 2H), 7.59 (d, J = 9.2 Hz, 1H), 7.47 (dd, J = 9.2 Hz, 2.0 Hz, 1H), 7.31 (dd, J = 9.2 Hz, 2.0 Hz, 1H), 6.57 (d, J = 8.0 Hz, 1H), 6.01 (tt, J_{HF} = 55.2 Hz, 4.0 Hz, 1H), 4.98 (s, 2H), 4.17 (s, 3H), 4.01 (td, J_{HF} = 14.4 Hz, 4.0 Hz, 2H). |
| | 1-(benzo[c]isothiazol-5-yl)-7- | |
| | (2,2-difluoroethoxy)-3-(2- | |
| | methyl-2H-indazol-5-yl)-3,4- | |
| | dihydropyrido[2,3- | |
| | d]pyrimidin-2(1H)-one | |
| Example 178 | 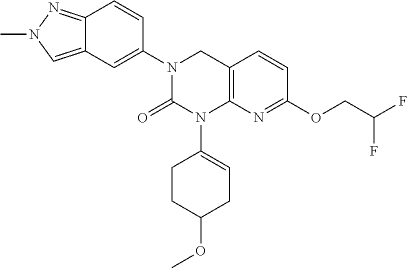 [SMILES: COC1CC=C(N2C(=O)N(c3ccc4nn(C)cc4c3)Cc3ccc(OCC(F)F)nc32)CC1] | LC-MS: m/z 470.2 (M + H)+. 1H NMR (400 MHz, DMSO-d₆) δ: 8.33 (s, 1H), 7.64 (d, J = 2.0 Hz, 1H), 7.60 (d, J = 8.0 Hz, 1H), 7.56 (d, J = 9.2 Hz, 1H), 7.21 (dd, J = 9.2 Hz, 1.6 Hz, 1H), 6.52 (d, J = 8.0 Hz, 1H), 6.37 (tt, J_{HF} = 55.2 Hz, 4.0 Hz, 1H), 5.63-5.59 (m, 1H), 4.81 (dd, J = 20.0 Hz, 14.0 Hz, 2H), 4.52 (td, J_{HF} = 14.8 Hz, 2.4 Hz, 2H), 4.17 (m, 3H), 3.57-3.50 (m, 1H), 3.29 (s, 3H), 2.52-2.19 (m, 3H), 2.16-2.06 (m, 1H), 2.01-1.92 (m, 1H), 1.80- 1.69 (m, 1H). |
| | 7-(2,2-difluoroethoxy)-1-(4- | |
| | methoxycyclohex-1-en-1-yl)-3- | |
| | (2-methyl-2H-indazol-5-yl)- | |
| | 3,4-dihydropyrido[2,3- | |
| | d]pyrimidin-2(1H)-one | |
| Example 179 | 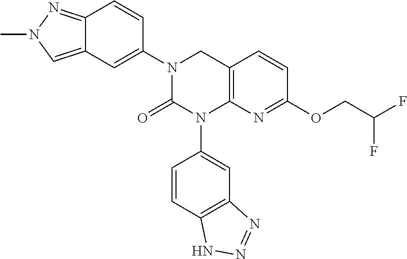 [SMILES: Cn1cc2cc(N3Cc4ccc(OCC(F)F)nc4N(c4ccc5[nH]nnc5c4)C3=O)ccc2n1] | LC-MS: m/z 477.2 (M + H)+. 1H NMR (400 MHz, DMSO-d₆) δ: 8.36 (s, 1H), 8.01-7.95 (m, 2H), 7.75-7.68 (m, 2H), 7.58 (d, J = 8.8 Hz, 1H), 7.42 (d, J = 8.8 Hz, 1H), 7.31 (dd, J = 9.2 Hz, 2.0 Hz, 1H), 6.56 (d, J = 8.4 Hz, 1H), 5.93 (tt, J_{HF} = 55.6 Hz, 4.0 Hz, 1H), 4.99 (s, 2H), 4.17 (s, 3H), 3.95 (td, J_{HF} = 14.4 Hz, 3.6 Hz, 2H), triazole proton did not appear. |
| | 1-(1H-benzo[d][1,2,3]triazol-5- | |
| | yl)-7-(2,2-difluoroethoxy)-3- | |
| | (2-methyl-2H-indazol-5-yl)- | |
| | 3,4-dihydropyrido[2,3- | |
| | d]pyrimidin-2(1H)-one | |

### General Procedure III

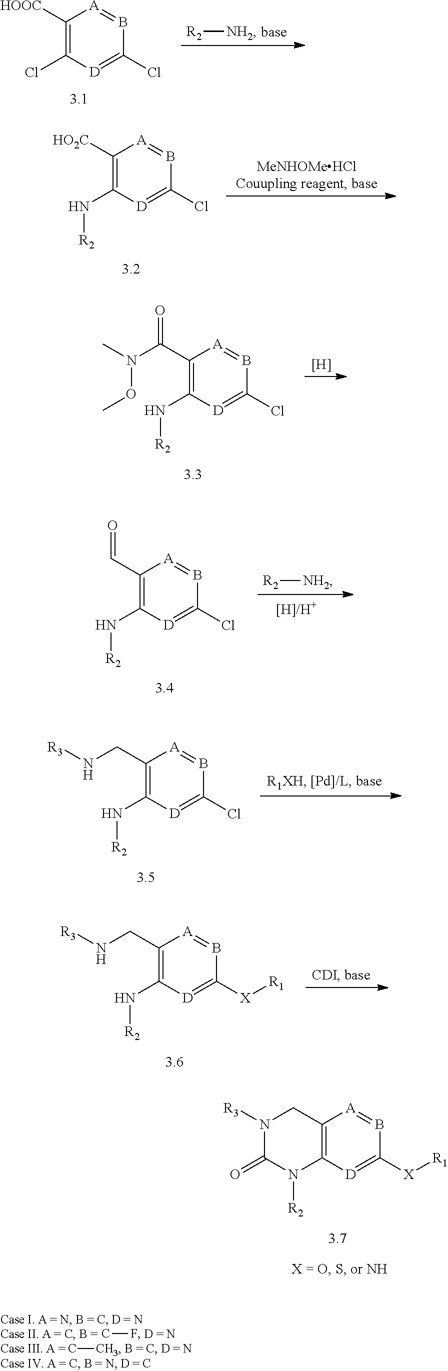

Compounds of structure 3.7 (cases I-IV) were obtained through the scheme depicted as General Procedure III. Beginning with aryl-chloride 3.1, the desired R² group was introduced using a base mediated aromatic substitution to generate amine 3.2. Aryl-acid 3.2 was then converted to Weinreb amide 3.3, which was then reduced to aldehyde 3.4 using a hydride source. The desired R₃ group was then introduced using reductive amination to generate diamine 3.5. The desired Ri group was then introduced with a palladium mediated C—X coupling reaction to generate diamine 3.6. Diamine 3.6 was then reacted with CDI to form cyclic urea 3.7.

### Preparation of Example 180 Via General Procedure III (Case I)

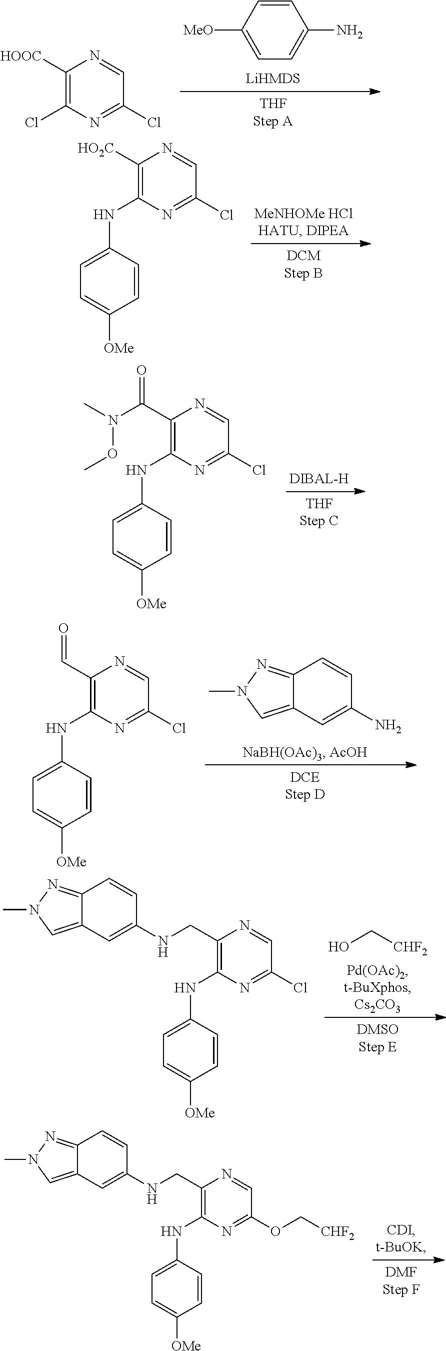

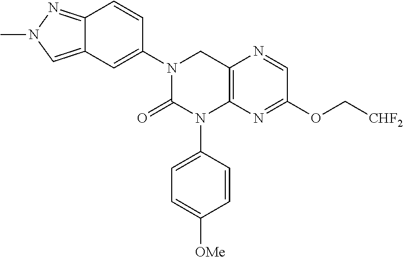 [SMILES: COc1ccc(N2C(=O)N(c3ccc4nn(C)cc4c3)Cc3ncc(OCC(F)F)nc32)cc1]

### Step A: 5-chloro-3-((4-methoxyphenyl)amino)pyrazine-2-carboxylic acid

A solution of 4-methoxyaniline (1.28 g, 10.4 mmol, 2.0 eq.) in anhy. THF (10 mL) was added LiHMDS (1M in THF, 10.4 mL, 10.4 mmol, 2.0 eq.) at −78° C. under N₂ atmosphere via a syringe. After stirring for an additional 0.5 hr at the same temperature, a solution of 3,5-dichloropyrazine-2-carboxylic acid (1.0 g, 5.2 mmol, 1.0 eq.) in anhy. THF (5 mL) was added via a syringe over 10 min. After stirring at −78° C. for additional 0.5 hr, the reaction mixture was allowed to warm to room temperature and stirred for 18 hrs. The mixture was quenched with water (20 mL), then adjusted pH=2 by the adding of dilute HCl (2N, aq.). The resulting mixture was extracted with EtOAc (10 mL×3). The combined organic layers were washed with brine (20 mL), dried over Na₂SO₄, concentrated under reduced pressure, and the residue was purified by flash column chromatography on silica gel to give 5-chloro-3-((4-methoxyphenyl)amino)pyrazine-2-carboxylic acid (1.3 g, 90% yield) as a yellow solid. LC-MS (ESI): m/z 280 [M+H]⁺.

### Step B: 5-chloro-N-methoxy-3-((4-methoxyphenyl)amino)-N-methylpyrazine-2-carboxamide

A solution of 5-chloro-3-((4-methoxyphenyl)amino)pyrazine-2-carboxylic acid (1.70 g, 6.08 mmol, 1.0 eq.), N, O-dimethylhydroxylamine hydrochloride (0.89 g, 9.12 mmol, 1.5 eq.), DIPEA (3.14 g, 24.31 mmol, 4.0 eq.) and HATU (3.46 g, 9.12 mmol, 1.5 eq.) in DCM (10 mL) was stirred at room temperature overnight. The resulting mixture was quenched by adding H₂O (20 mL), then extracted with DCM (30 mL×3). The combined organic layer was washed with brine (30 mL) and dried over Na₂SO₄, concentrated under reduced pressure, and the residue was purified by flash column chromatography on silica gel to give 5-chloro-N-methoxy-3-((4-methoxyphenyl)amino)-N-methylpyrazine-2-carboxamide (1.26 g, 64% yield) as a yellow solid. LC-MS (ESI): m/z 323 [M+H]⁺.

### Step C: 5-chloro-3-((4-methoxyphenyl)amino)pyrazine-2-carbaldehyde

A solution of 5-chloro-N-methoxy-3-((4-methoxyphenyl)amino)-N-methylpyrazine-2-carboxamide (1.26 g, 3.9 mmol, 1.0 eq.) in anhy. THF (15 mL) was added DIBAL-H (1.5 M in toluene, 3.9 mL, 5.86 mmol, 1.5 eq.) at −78° C. under N₂ atmosphere via a syringe. The mixture was stirred at −78° C. for additional 0.5 hr. the reaction progress was monitored by LC-MS, after completion, the reaction mixture was quenched with NH₄Cl (sat. aq.) (20 mL) and extracted with EtOAc (10 mL×3). The combined organic layers were washed with brine (30 mL) and dried over Na₂SO₄, concentrated under reduced pressure to give 5-chloro-3-((4-methoxyphenyl)amino)pyrazine-2-carbaldehyde (1.6 g, crude), which was used in next step without further purification. LC-MS (ESI): m/z 264 [M+H]⁺.

### Step D: N-((5-chloro-3-((4-methoxyphenyl)amino)pyrazin-2-yl)methyl)-2-methyl-2H-indazol-5-amine

A solution of 5-chloro-3-((4-methoxyphenyl)amino)pyrazine-2-carbaldehyde (1.6 g, 6.07 mmol, 1.0 eq.), 2-methyl-2H-indazol-5-amine (0.89 g, 6.07 mmol, 1.0 eq.) and AcOH (1.5 mL, 24.27 mmol, 4.0 eq.) in DCE (10 mL) was stirred at room temperature overnight. Then NaBH(OAc)₃ (3.86 g, 18.20 mmol, 3.0 eq.) was added in several potions at 0° C., after addition, the mixture was allowed to warm to room temperature and stirred overnight. The reaction was quenched by adding ice water (10 mL) and extracted with EtOAc (30 mL×3). The combined organic layers were washed with brine (30 mL) and dried over Na₂SO₄, washed with brine (30 mL) and dried over Na₂SO₄, concentrated under reduced pressure, the residue was purified by flash column chromatography on silica gel to give N-((5-chloro-3-((4-methoxyphenyl)amino)pyrazin-2-yl)methyl)-2-methyl-2H-indazol-5-amine (1.25 g, 52% yield) as a yellow solid. LC-MS (ESI): m/z 395 [M+H]⁺.

### Step E: N-((5-(2,2-difluoroethoxy)-3-((4-methoxyphenyl)amino)pyrazin-2-yl)methyl)-2-methyl-2H-indazol-5-amine

The solution of N-((5-chloro-3-((4-methoxyphenyl)amino)pyrazin-2-yl)methyl)-2-methyl-2H-indazol-5-amine (600 mg, 1.52 mmol, 1.0 eq.), 2,2-difluoroethan-1-ol (374 mg, 4.56 mmol, 3.0 eq.), Pd(OAc)₂ (68 mg, 0.304 mmol, 0.2 eq.), t-BuXPhos (129 mg, 0.304 mmol, 0.2 eq.) and Cs₂CO₃ (1.49 g, 4.56 mmol, 3.0 eq.) in DMSO (10 mL) was stirred at 100° C. overnight in a sealed tube under N₂ atmosphere. The resulting mixture was concentrated and the residue was purified by silica gel column to give N-((5-(2,2-difluoroethoxy)-3-((4-methoxyphenyl)amino)pyrazin-2-yl)methyl)-2-methyl-2H-indazol-5-amine (250 mg, 37% yield) as a yellow solid. LC-MS (ESI): m/z 441 [M+H]⁺.

### Step F: 7-(2,2-difluoroethoxy)-1-(4-methoxyphenyl)-3-(2-methyl-2H-indazol-5-yl)-3,4-dihydropteridin-2(1H)-one

The solution of N-((5-(2,2-difluoroethoxy)-3-((4-methoxyphenyl)amino)pyrazin-2-yl)methyl)-2-methyl-2H-indazol-5-amine (280 mg, 0.64 mmol, 1.0 eq.), CDI (206 mg, 1.27 mmol, 2.0 eq.) and t-BuOK (285 mg, 2.55 mmol, 4.0 eq.) in DMF (10 mL) was stirred at 50° C. for 4 hrs. The resulting mixture was concentrated and the residue was purified by RP-prep-HPLC to give 7-(2,2-difluoroethoxy)-1-(4-methoxyphenyl)-3-(2-methyl-2H-indazol-5-yl)-3,4-dihydropteridin-2(1H)-one (Example 180).

¹H NMR (400 MHz, DMSO-d₆) δ (ppm): 8.36 (s, 1H), 7.93 (s, 1H), 7.76 (d, J=1.2 Hz, 1H), 7.59 (d, J=9.2 Hz, 1H), 7.37-7.22 (m, 3H), 7.04-6.99 (d, J=8.8 Hz, 2H), 6.15 (tt, J_{HF}=54.8 Hz, J=3.6 Hz, 1H), 5.03 (s, 2H), 4.30-4.03 (s, 2H), 4.18 (s, 3H), 3.80 (s, 3H).

LC-MS (ESI): m/z 467 [M+H]⁺.

The procedure set forth above for General Procedure III was used to synthesize the following compounds by using appropriate starting materials:

| Cpd No. | Structure | Characterization |
|---|---|---|
| Example 181 (case I) | 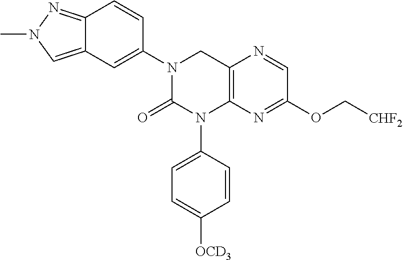 | LC-MS (ESI): m/z 470.2 [M + H]+. 1H NMR (400 MHz, DMSO-d₆) δ: 8.36 (s, 1H), 7.93 (s, 1H), 7.76 (d, J = 1.6 Hz, 1H), 7.59 (d, J = 9.2 Hz, 1H), 7.37-7.18 (m, 3H), 7.01 (d, J = 8.8 Hz, 2H), 6.15 (tt, J_{HF} = 55.2 Hz, J = 3.6 Hz, 1H), 5.03 (s, 2H), 4.29-4.01 (m, 2H), 4.17 (s, 3H). |
| | 7-(2,2-difluoroethoxy)-1-(4-(methoxy- | |
| | d3)phenyl)-3-(2-methyl-2H-indazol-5- | |
| | yl)-3,4-dihydropteridin-2(1H)-one | |
| Example 182 (case I) | 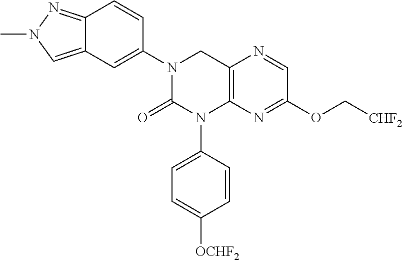 [SMILES: Cn1cc2cc(N3Cc4ncc(OCC(F)F)nc4N(c4ccc(OC(F)F)cc4)C3=O)ccc2n1] | LC-MS (ESI): m/z 503.0 [M + H]+. 1H NMR (400 MHz, DMSO-d₆) δ: 8.37 (s, 1H), 7.96 (s, 1H), 7.77 (d, J = 1.6 Hz, 1H), 7.60 (d, J = 9.2 Hz, 1H), 7.46 (d, J = 8.8 Hz, 2H), 7.32-7.27 (m, 3H), 7.31 (t, J_{HF} = 73.8 Hz, 1H), 6.15 (tt, J_{HF} = 56 Hz, J = 3.6 Hz, 1H), 5.04 (s, 2H), 4.18 (td, J_{HF} = 14.8 Hz, J = 3.6 Hz, 2H), 4.17 (s, 3H). |
| | 7-(2,2-difluoroethoxy)-1-(4- | |
| | (difluoromethoxy)phenyl)-3-(2-methyl- | |
| | 2H-indazol-5-yl)-3,4-dihydropteridin- | |
| | 2(1H)-one | |
| Example 183 (case I) | 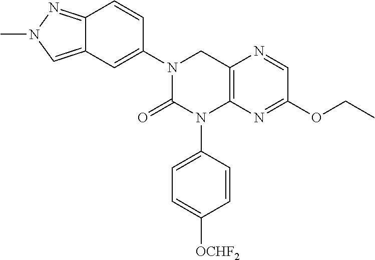 [SMILES: CCOc1cnc2c(n1)N(c1ccc(OC(F)F)cc1)C(=O)N(c1ccc3nn(C)cc3c1)C2] | LC-MS (ESI): m/z 467.0 [M + H]+. 1H NMR (400 MHz, DMSO-d₆) δ: 8.37 (s, 1H), 7.83 (s, 1H), 7.77 (s, 1H), 7.60 (d, J = 9.2 Hz, 1H), 7.45 (d, J = 8.4 Hz, 2H), 7.32 (t, J_{HF} = 74.8 Hz, 1H), 7.30-7.26 (m, 3H), 5.02 (s, 2H), 4.18 (s, 3H), 3.97 (q, J = 6.8 Hz, 2H), 1.13 (t, J = 6.8 Hz, 3H). |
| | 1-(4-(difluoromethoxy)phenyl)-7- | |
| | ethoxy-3-(2-methyl-2H-indazol-5-yl)- | |
| | 3,4-dihydropteridin-2(1H)-one | |
| Example 184 (case II) Using 2,6- dichloro-5- fluoronicotinic acid as the starting material. | 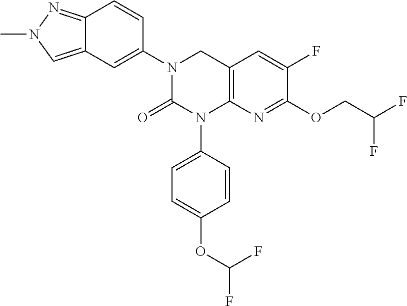 [SMILES: Cn1cc2cc(N3Cc4cc(F)c(OCC(F)F)nc4N(c4ccc(OC(F)F)cc4)C3=O)ccc2n1] | LC-MS: m/z 520.1 (M + H)+. 1H NMR (400 MHz, DMSO-d₆) δ: 8.36 (s, 1H), 7.77 (d, J = 2.0 Hz, 1H), 7.70 (d, J = 2.0 Hz, 1H), 7.58 (d, J = 9.2 Hz, 1H), 7.43 (d, J = 9.2 Hz, 2H), 7.28 (t, J_{HF} = 74.0 Hz, 1H), 7.29-7.23 (m, 3H), 6.07 (tt, J_{HF} = 55.2 Hz, 4.0 Hz, 1H), 4.94 (s, 2H), 4.17 (s, 3H), 4.15 (td, J_{HF} = 14.4 Hz, 4.0 Hz, 2H). |
| | (2,2-difluoroethoxy)-1-(4- | |
| | (difluoromethoxy)phenyl)-6-fluoro-3- | |
| | (2-methyl-2H-indazol-5-yl)-3,4- | |
| | dihydropyrido[2,3-d]pyrimidin-2(1H)- | |
| | one | |
| Example 185 (case III) Using 2,6- dichloro-4- methylnico- tinic acid as the starting material. | 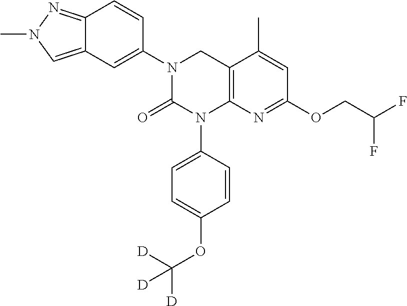 [SMILES: [2H]C([2H])([2H])Oc1ccc(N2C(=O)N(c3ccc4nn(C)cc4c3)Cc3c(C)cc(OCC(F)F)nc32)cc1] | LC-MS: m/z 483.2 (M + H)+. 1H NMR (400 MHz, DMSO-d₆) δ: 8.35 (s, 1H), 7.73 (d, J = 1.6 Hz, 1H), 7.58 (d, J = 9.2 Hz, 1H), 7.28 (dd, J = 9.2 Hz, 2.0 Hz, 1H), 7.22 (d, J = 8.8 Hz, 1H), 6.98 (d, J = 8.4 Hz, 2H), 6.41 (s, 1H), 5.97 (tt, J_{HF} = 55.6 Hz, 4.0 Hz, 1H), 4.88 (s, 2H), 4.17 (s, 3H), 4.03 (td, J_{HF} = 14.4 Hz, 4.0 Hz, 2H), 2.21 (s, 3H). |
| | 7-(2,2-difluoroethoxy)-1-(4-methoxy-d3- | |
| | phenyl)-5-methyl-3-(2-methyl-2H- | |
| | indazol-5-yl)-3,4-dihydropyrido[2,3- | |
| | d]pyrimidin-2(1H)-one | |
| Example 186 (case I) | 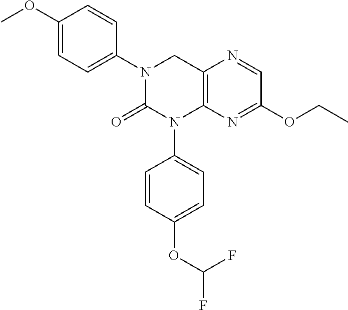 [SMILES: CCOc1cnc2c(n1)N(c1ccc(OC(F)F)cc1)C(=O)N(c1ccc(OC)cc1)C2] | LC-MS: m/z 443.2 (M + H)+. 1H NMR (400 MHz, DMSO-d₆) δ: 7.81 (s, 1H), 7.42 (d, J = 8.8 Hz, 2H), 7.38 (d, J = 8.8 Hz, 2H), 7.31 (t, J_{HF} = 74.0 Hz, 1H), 7.26 (d, J = 8.8 Hz, 2H), 6.97 (d, J = 8.8 Hz, 2H), 4.93 (s, 2H), 3.95 (q, J = 7.2 Hz, 2H), 3.77 (s, 3H), 1.12 (t, J = 7.2 Hz, 3H). |
| | 1-(4-(difluoromethoxy)phenyl)-7- | |
| | ethoxy-3-(4-methoxyphenyl)-3,4- | |
| | dihydropteridin-2(1H)-one | |
| Example 187 (case IV) Using 4,6- dichloronico- tinic acid as the starting material. | 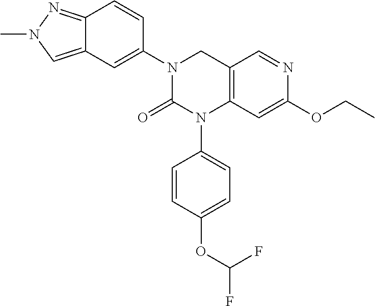 [SMILES: CCOc1cc2c(cn1)CN(c1ccc3nn(C)cc3c1)C(=O)N2c1ccc(OC(F)F)cc1] | LC-MS: m/z 466.2 (M + H)+. 1H NMR (400 MHz, DMSO-d₆) δ: 8.35 (s, 1H), 7.99 (s, 1H), 7.70 (d, J = 1.2 Hz, 1H), 7.57 (d, J = 9.2 Hz, 1H), 7.46 (d, J = 8.8 Hz, 2H), 7.35 (d, J = 8.8 Hz, 2H), 7.34 (t, J_{HF} = 74.0 Hz, 1H), 7.28 (dd, J = 9.2 Hz, 2.0 Hz, 1H), 5.33 (s, 1H), 4.96 (s, 2H), 4.21 (q, J = 7.2 Hz, 2H), 4.17 (s, 3H), 1.21 (t, J = 7.2 Hz, 3H). |
| | 1-(4-(difluoromethoxy)phenyl)-7- | |
| | ethoxy-3-(2-methyl-2H-indazol-5-yl)- | |
| | 3,4-dihydropyrido[4,3-d]pyrimidin- | |
| | 2(1H)-one | |
| Example 413 (case I) | 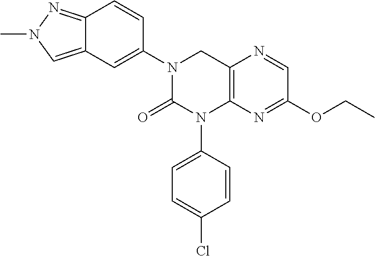 [SMILES: CCOc1cnc2c(n1)N(c1ccc(Cl)cc1)C(=O)N(c1ccc3nn(C)cc3c1)C2] | LC-MS: m/z 435 (M + H)+. 1H NMR (400 MHz, DMSO-d₆) δ: 8.35 (s, 1H), 7.82 (s, 1H), 7.75 (s, 1H), 7.58 (d, J = 9.2 Hz, 1H), 7.52 (d, J = 8.6 Hz, 2H), 7.41 (d, J = 8.6 Hz, 2H), 7.29 (dd, J = 9.1 Hz, 1.6 Hz, 1H), 5.00 (s, 2H), 4.16 (s, 3H), 3.95 (q, J = 7.0 Hz, 2H), 1.13 (t, J = 7.0 Hz, 3H). |
| | 1-(4-chlorophenyl)-7-ethoxy-3-(2- | |
| | methyl-2H-indazol-5-yl)-3,4- | |
| | dihydropteridin-2(1H)-one | |
| Example 414 (case I) | 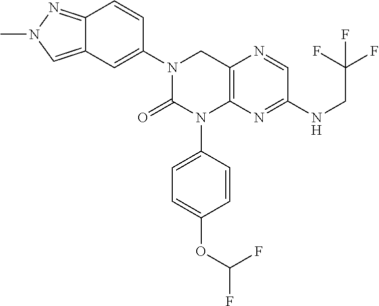 [SMILES: Cn1cc2cc(N3Cc4ncc(NCC(F)(F)F)nc4N(c4ccc(OC(F)F)cc4)C3=O)ccc2n1] | LC-MS: m/z 520 (M + H)+. 1H NMR (400 MHz, DMSO-d₆) δ: 8.41 (s, 1H), 7.80 (s, 1H), 7.68 (s, 1H), 7.64 (d, J = 8.8 Hz, 2H), 7.43 (d, J = 8.4 Hz, 2H), 7.36 (d, J = 9.0 Hz, 1H), 7.32 (t, J_{HF} = 76.2 Hz, 1H), 7.30 (d, J = 8.4 Hz, 2H), 4.97 (s, 2H), 4.23 (s, 3H), 3.83-3.74 (m, 2H). |
| | 1-(4-(difluoromethoxy)phenyl)-3-(2- | |
| | methyl-2H-indazol-5-yl)-7-((2,2,2- | |
| | trifluoroethyl)amino)-3,4- | |
| | dihydropteridin-2(1H)-one | |
| Example 415 (case I) | 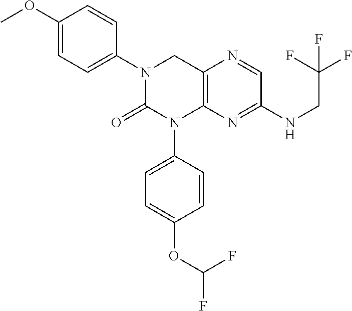 [SMILES: COc1ccc(N2Cc3ncc(NCC(F)(F)F)nc3N(c3ccc(OC(F)F)cc3)C2=O)cc1] | LC-MS: m/z 496 (M + H)+. 1H NMR (400 MHz, DMSO-d₆) δ: 7.62 (s, 1H), 7.57 (t, J = 6.4 Hz, 1H), 7.39-7.31 (m, 4H), 7.27 (t, J_{HF} = 74.4 Hz, 1H), 7.23 (d, J = 9.2 Hz, 2H), 6.96 (d, J = 8.8 Hz, 2H), 4.83 (s, 2H), 3.77 (s, 3H), 3.75-3.68 (m, 2H). |
| | 1-(4-(difluoromethoxy)phenyl)-3-(4- | |
| | methoxyphenyl)-7-((2,2,2- | |
| | trifluoroethyl)amino)-3,4- | |
| | dihydropteridin-2(1H)-one | |
| Example 416 (case I) | 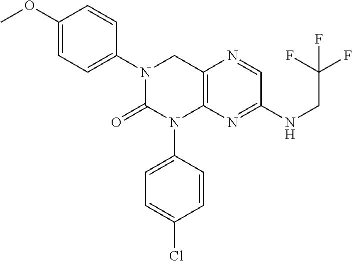 [SMILES: COc1ccc(N2Cc3ncc(NCC(F)(F)F)nc3N(c3ccc(Cl)cc3)C2=O)cc1] | LC-MS: m/z 464 (M + H)+. 1H NMR (400 MHz, DMSO-d₆) δ: 7.63 (s, 1H), 7.59 (t, J = 6.4 Hz, 1H), 7.49 (d, J = 8.4 Hz, 2H), 7.39-7.30 (m, 4H), 6.97 (d, J = 8.8 Hz, 2H), 4.83 (s, 2H), 3.77 (s, 3H), 3.76-3.69 (m, 2H). |
| | 1-(4-chlorophenyl)-3-(4- | |
| | methoxyphenyl)-7-((2,2,2- | |
| | trifluoroethyl)amino)-3,4- | |
| | dihydropteridin-2(1H)-one | |
| Example 417 (case I) | 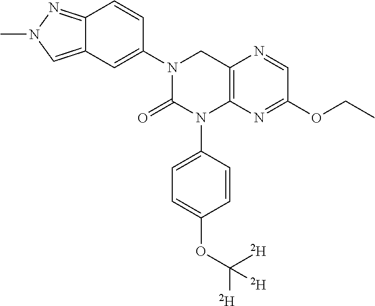 [SMILES: [2H]C([2H])([2H])Oc1ccc(N2C(=O)N(c3ccc4nn(C)cc4c3)Cc3ncc(OCC)nc32)cc1] | LC-MS: m/z 434 (M + H)+. 1H NMR (400 MHz, DMSO-d₆) δ: 8.36 (s, 1H), 7.80 (s, 1H), 7.75 (s, 1H), 7.59 (d, J = 9.2 Hz, 1H), 7.32-7.24 (m, 3H), 7.00 (d, J = 8.8 Hz, 2H), 4.99 (s, 2H), 4.17 (s, 3H), 3.96 (q, J = 6.8 Hz, 2H), 1.14 (t, J = 7.2 Hz, 3H). |
| | 7-ethoxy-1-(4-(methoxy-d3)phenyl)-3- | |
| | (2-methyl-2H-indazol-5-yl)-3,4- | |
| | dihydropteridin-2(1H)-one | |
| Example 418 (case IV) | 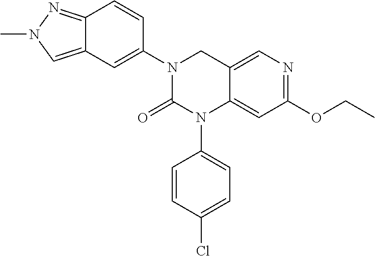 [SMILES: CCOc1cc2c(cn1)CN(c1ccc3nn(C)cc3c1)C(=O)N2c1ccc(Cl)cc1] | LC-MS: m/z 434 (M + H)+. 1H NMR (400 MHz, DMSO-d₆) δ: 8.35 (s, 1H), 7.99 (s, 1H), 7.70 (s, 1H), 7.62 (d, J = 7.6 Hz, 2H), 7.57 (d, J = 9.2 Hz, 1H), 7.44 (d, J = 8.0 Hz, 2H), 7.28 (d, J = 9.2 Hz, 1H), 5.35 (s, 1H), 4.96 (s, 2H), 4.21 (q, J = 6.8 Hz, 2H), 4.17 (s, 3H), 1.22 (t, J = 6.8 Hz, 3H). |
| | 1-(4-chlorophenyl)-7-ethoxy-3-(2- | |
| | methyl-2H-indazol-5-yl)-3,4- | |
| | dihydropyrido[4,3-d]pyrimidin-2(1H)- | |
| | one | |

### General Procedure IV

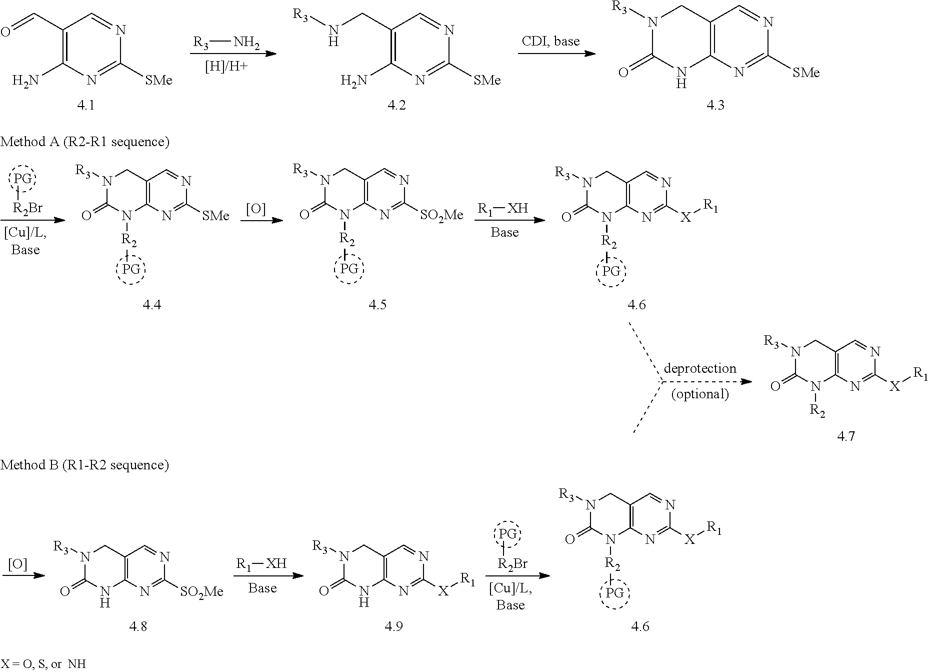

Compounds of structure 4.7 were obtained through the scheme depicted as General Procedure IV. Beginning with aldehyde 4.1, the desired R₃ group was introduced using a reductive amination to generate diamine 4.2. Diamine 4.2 was subjected to CDI to form cyclic urea 4.3. The desired R₁ and R₂ were then installed in one of two ways. In Method A, the desired R₂ group (optionally protected) was introduced by a copper mediated C—N coupling reaction with urea 4.3 to generate compound 4.4. Thiol 4.4 was then oxidized to sulfone 4.5 and then displaced with the desired R₁ using a base mediated aromatic substitution reaction to afford compound 4.6. If necessary, compound 4.6 was then deprotected to afford compound 4.7. In Method B, thiol 4.3 was oxidized to sulfone 4.8, which was then displaced with the desired R₁ using a base mediated aromatic substitution reaction to afford compound 4.9. The desired R₂ group (optionally protected) was then introduced by a copper mediated C—N coupling reaction with urea 4.9 to generate compound 4.6. If necessary, compound 4.6 was then deprotected to afford compound 4.7.

### Preparation of Example 188 Via General Procedure IV (Method A)

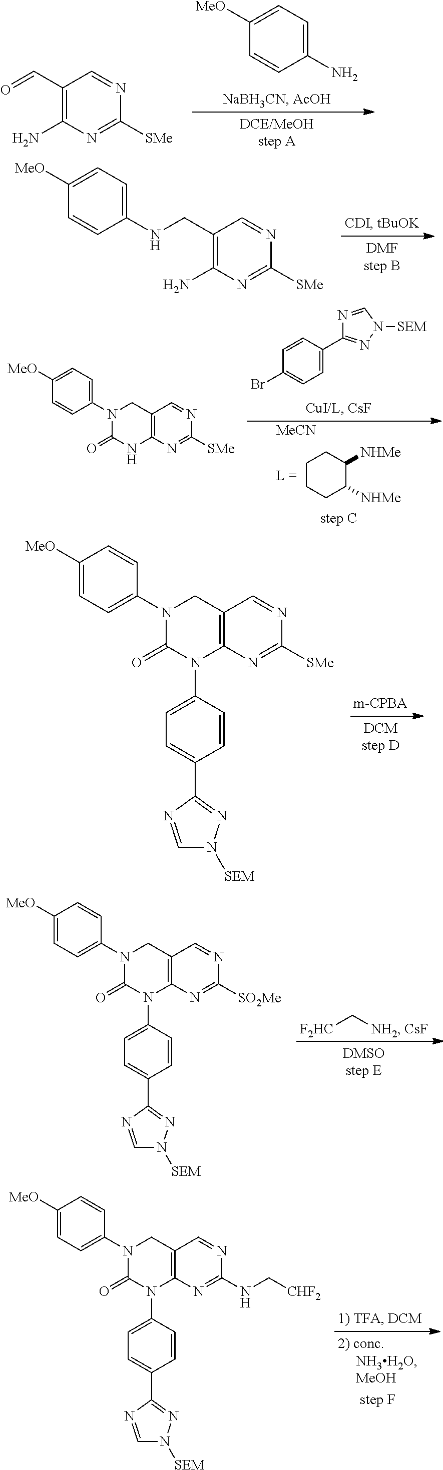

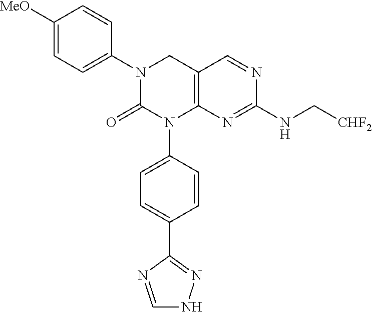 [SMILES: COc1ccc(N2Cc3cnc(NCC(F)F)nc3N(c3ccc(-c4nc[nH]n4)cc3)C2=O)cc1]

### Step A: 5-(((4-methoxyphenyl)amino)methyl)-2-(methylthio)pyrimidin-4-amine

4-Amino-2-(methylthio)pyrimidine-5-carbaldehyde (1.14 g, 6.74 mmol, 1.0 eq.), 4-methoxyaniline (0.91 g, 7.42 mmol, 1.1 eq.) and AcOH (1.2 mL, 20.23 mmol, 3.0 eq.) was dissolved in DCE/MeOH (6 mL/6 mL), stirred at room temperature for 3 hrs. then NaBH₃CN (0.47 g, 7.42 mmol, 1.1 eq.) was in several portions at 0° C., then the reaction mixture was allowed to warm to room temperature and stirred overnight. The reaction quenched with ice water (10 mL), and extracted with DCM (20 mL×3), the combined organic layers were dried over Na₂SO₄, concentrated under reduced pressure, the residue was purified by flash column chromatography on silica gel to give 5-(((4-methoxyphenyl)amino)methyl)-2-(methylthio)pyrimidin-4-amine (1.7 g, 91% yield) as a gray solid. LC-MS (ESI): m/z 277 [M+H]⁺.

### Step B: 3-(4-methoxyphenyl)-7-(methylthio)-3,4-dihydropyrimido[4,5-d]pyrimidin-2(1H)-one

5-(((4-Methoxyphenyl)amino)methyl)-2-(methylthio)pyrimidin-4-amine (0.83 g, 3.0 mmol, 1.0 eq.) was dissolved in anhy. DMF (20 mL). t-BuOK (1.35 g, 1.2 mol, 4.0 eq.) and CDI (0.97 g, 6.01 mmol, 2.0 eq.) were added in several portions. The reaction mixture was heated to 50° C. and stirred for 4 hrs. The reaction mixture was treated with ice water (20 mL), the resulting precipitate was collected and washed with H₂O (10 mL×3), dried under reduced pressure to afford 3-(4-methoxyphenyl)-7-(methylthio)-3,4-dihydropyrimido[4,5-d]pyrimidin-2(1H)-one

5-(((4-Methoxyphenyl)amino)methyl)-2-(methylthio)pyrimidin-4-amine (0.69 g, 76% yield) as a white solid. LC-MS (ESI): m/z 303 [M+H]⁺.

### Step C: 3-(4-methoxyphenyl)-7-(methylthio)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one

To a solution of 3-(4-methoxyphenyl)-7-(methylthio)-3,4-dihydropyrimido[4,5-d]pyrimidin-2(1H)-one (604 mg, 2.0 mmol, 1.0 eq.) in MeCN (8 mL) was added CuI (190 mg, 1.0 mmol, 0.5 eq.), CsF (912 mg, 6.0 mmol, 3.0 eq.), N¹,N²-dimethylcyclohexane-1,2-diamine (284 mg, 2.0 mmol, 1.0 eq.) and 3-(4-bromophenyl)-1-((2-(trimethylsilyl)ethoxy)methyl)-1H-1,2,4-triazole (Ref: WO2008156726 A1) (850 mg, 2.4 mmol, 1.2 eq.) at room temperature and the mixture stirred at 100° C. for 16 hrs. Then the reaction mixture was quenched with ice water (20 mL) and extracted with EtOAc (20 mL×3). The combined organic layers were washed with brine (50 mL), dried over Na₂SO₄ and concentrated under reduced pressure, the residue mixture was purified by flash column chromatography on silica gel to give 3-(4-methoxyphenyl)-7-(methylthio)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (580 mg, 50% yield) as a white solid. LC-MS (ESI): m/z 576 [M+H]⁺.

### Step D: 3-(4-methoxyphenyl)-7-(methylsulfonyl)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one

To a solution of 3-(4-methoxyphenyl)-7-(methylthio)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (676 mg, 1.1 mmol, 1.0 eq.) in DCM (10 mL) was added m-CPBA (571 mg, 3.3 mmol, 3.0 eq.) in several portions at room temperature. The resulting mixture was stirred at room temperature for additional 2 hrs. Then the excess of m-CPBA was quenched with NaHSO₃ (sat. aq.) (30 mL), the resulting mixture was extracted with DCM (10 mL×3). The combined organic layers were washed with brine (30 mL), dried over Na₂SO₄ and concentrated under reduced pressure. The residue was purified by flash column chromatography on silica gel to afford 3-(4-methoxyphenyl)-7-(methylsulfonyl)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (600 mg, 90% yield) as a white solid. LC-MS (ESI): m/z 608 [M+H]⁺.

### Step E: 7-((2,2-difluoroethyl)amino)-3-(4-methoxyphenyl)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one

To a solution of 3-(4-methoxyphenyl)-7-(methylsulfonyl)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (130 mg, 0.21 mmol, 1.0 eq.) in DMSO (5 mL) was added CsF (32 mg, 0.21 mmol, 1.0 eq.), DIPEA (0.18 mL, 1.07 mmol, 5.0 eq.) and 2,2,2-trifluoroethanamine (109 mg, 1.1 mmol, 5.0 eq.) at room temperature. The resulting mixture was stirred at 80° C. for 16 hrs in a sealed tube. Then the reaction mixture was quenched with ice water (10 mL) and extracted with DCM (10 mL×3). The combined organic layers were washed with brine (15 mL), dried over Na₂SO₄ and concentrated under reduced pressure, the residue was purified by flash column chromatography on silica gel to give 7-((2,2-difluoroethyl)amino)-3-(4-methoxyphenyl)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (30 mg, 23% yield) as a white solid. LC-MS (ESI): m/z 609 [M+H]⁺.

### Step F: 1-(4-(1H-1,2,4-triazol-3-yl)phenyl)-7-((2,2-difluoroethyl)amino)-3-(4-methoxyphenyl)-3,4-dihydropyrimido[4,5-d]pyrimidin-2(1H)-one

To solution of 7-((2,2-difluoroethyl)amino)-3-(4-methoxyphenyl)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (30 mg, 0.05 mmol, 1.0 eq.) in DCM (2 mL) was added TFA (0.5 mL) at 0° C. Then the reaction mixture was stirred at room temperature for 5 hrs. Most of solvents were evaporated under reduced pressure. The residue was diluted with MeOH (2 mL), added conc. NH₄OH (0.5 mL) and stirred for 2 hrs at room temperature. Then the reaction mixture was concentrated under reduced pressure. The residue was purified by RP-prep-HPLC to afford 1-(4-(1H-1,2,4-triazol-3-yl)phenyl)-7-((2,2-difluoroethyl)amino)-3-(4-methoxyphenyl)-3,4-dihydropyrimido[4,5-d]pyrimidin-2(1H)-one (Example 188).

¹H NMR (400 MHz, DMSO-d₆) δ (ppm): 14.23 (bs, 1H), 8.69-8.35 (m, 1H), 8.12-8.07 (m, 3H), 7.43 (d, J=8.0 Hz, 2H), 7.69-7.08 (m, 1H), 7.36 (d, J=8.8 Hz, 2H), 6.98 (d, J=8.8 Hz, 2H), 5.88 (bs, 1H), 4.78 (s, 2H), 3.78 (s, 3H), 3.70-3.50 (m, 2H).

LC-MS (ESI): m/z 479 [M+H]⁺.

### Preparation of Example 189 Via General Procedure IV (Method B)

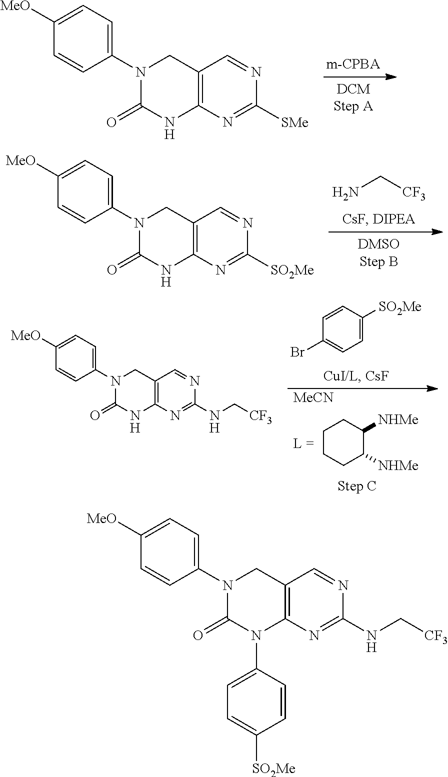

### Step A: 3-(4-methoxyphenyl)-7-(methylsulfonyl)-3,4-dihydropyrimido[4,5-d]pyrimidin-2(1H)-one

To a solution of 3-(4-methoxyphenyl)-7-(methylthio)-3,4-dihydropyrimido[4,5-d]pyrimidin-2(1H)-one (0.8 g, 2.65 mmol, 1.0 eq.) in DCM (20 mL) was added 3-chlorobenzoperoxoic acid (2.28 g, 13.24 mmol, 5.0 eq.) in several portions at room temperature. The mixture was stirred at 40° C. for 3 hrs. The reaction mixture was quenched with NaHSO₃ (sat. aq) (50 mL) to consume the excess m-CPBA, Then the reaction mixture was quenched with ice water (20 mL) and extracted with EtOAc (20 mL×3). The combined organic layers were washed with brine (50 mL), dried over Na₂SO₄ and concentrated under reduced pressure, and the residue mixture was purified by flash column chromatography on silica gel to give 3-(4-methoxyphenyl)-7-(methylsulfonyl)-3,4-dihydropyrimido[4,5-d]pyrimidin-2(1H)-one as a white solid (0.8 g, 90% yield). LC-MS (ESI): m/z 335 [M+H]⁺.

### Step B: 3-(4-methoxyphenyl)-7-((2,2,2-trifluoroethyl)amino)-3,4-dihydropyrimido[4,5-d]pyrimidin-2(1H)-one

A mixture of 3-(4-methoxyphenyl)-7-(methylsulfonyl)-3,4-dihydropyrimido[4,5-d]pyrimidin-2(1H)-one (0.1 g, 0.3 mmol, 1.0 eq.), CsF (0.14 g, 0.9 mmol, 3.0 eq.), DIPEA (0.19 g, 1.497 mmol, 5.0 eq.), and 2,2,2-trifluoroethan-1-amine (0.59 g, 5.99 mmol, 20 eq.) in DMSO (2 mL) was stirred at 80° C. for 24 hrs in a sealed tube. Then excess of 2,2,2-trifluoroethan-1-amine was removed under reduced pressure, ice water (20 mL) was added and extracted with EtOAc (10 mL×3). The combined organic layers were washed with brine (10 mL), dried over Na₂SO₄ and concentrated under reduced pressure, the residue mixture was purified by flash column chromatography on silica gel to give 3-(4-methoxyphenyl)-7-((2,2,2-trifluoroethyl)amino)-3,4-dihydropyrimido[4,5-d]pyrimidin-2(1H)-one as a brown solid (55 mg, 52% yield). LC-MS (ESI): m/z 354 [M+H]⁺.

### Step C: 3-(4-methoxyphenyl)-1-(4-(methylsulfonyl)phenyl)-7-((2,2,2-trifluoroethyl)amino)-3,4-dihydropyrimido[4,5-d]pyrimidin-2(1H)-one

To the mixture of 3-(4-methoxyphenyl)-7-((2,2,2-trifluoroethyl)amino)-3,4-dihydropyrimido[4,5-d]pyrimidin-2(1H)-one (35 mg, 0.1 mmol, 1.0 eq.) and 1-bromo-4-(methylsulfonyl)benzene (47 mg, 0.2 mmol, 2.0 eq.) in MeCN (2 mL) was added N¹,N²-dimethylcyclohexane-1,2-diamine (17 mg, 0.1 mmol, 1.0 eq.), CsF (27 mg, 0.2 mmol, 2.0 eq.) and CuI (11 mg, 0.1 mmol, 1.0 eq.). The mixture was stirred at 110° C. under microwave irradiation for 1 hr under N₂ atmosphere. The mixture was cooled to room temperature and quenched with water (15 mL) and extracted with EtOAc (20 mL×3). The combined organic layers were dried over Na₂SO₄, concentrated under reduced pressure, and the residue was purified by RP-prep-HPLC to afford 3-(4-methoxyphenyl)-1-(4-(methylsulfonyl)phenyl)-7-((2,2,2-trifluoroethyl)amino)-3,4-dihydropyrimido[4,5-d]pyrimidin-2(1H)-one (Example 189).

¹H NMR (400 MHz, CDCl₃) δ (ppm): 8.04 (s, 1H), 8.03 (d, J=8.6 Hz, 2H), 7.53 (d, J=8.5 Hz, 2H), 7.28 (d, J=8.9 Hz, 2H), 6.94 (d, J=8.9 Hz, 2H), 5.23 (bs, 1H), 4.79 (s, 2H), 4.25-3.54 (m, 2H), 3.08 (s, 3H), 2.35 (s, 3H).

LC-MS (ESI): m=z 508 [M+H]⁺.

The procedure set forth above for General Procedure IV (Method A) was used to synthesize the following compounds by using appropriate starting materials:

| Cpd No. | Structure | Characterization |
|---|---|---|
| Example 190 | 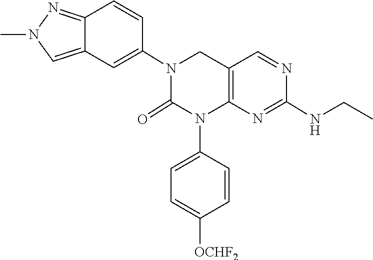 [SMILES: CCNc1ncc2c(n1)N(c1ccc(OC(F)F)cc1)C(=O)N(c1ccc3nn(C)cc3c1)C2] | LC-MS (ESI): m/z 466.0 [M + H]+. 1H NMR (400 MHz, DMSO-d₆) δ: 8.35 (s, 1H), 8.06 (s, 1H), 7.69 (d, J = 1.2 Hz, 1H), 7.57 (d, J = 8.8 Hz, 1H), 7.37 (d, J = 8.8 Hz, 2H), 7.30 (t, J_{HF} = 74.0 Hz, 1H), 7.26 (dd, J = 9.2 Hz, 2.0 Hz, 1H), 7.23 (d, J = 8.8 Hz, 2H), 6.83 (bs, 1H), 4.80 (s, 2H), 4.17 (s, 3H), 3.14-2.92 (m, 2H), 0.99-0.83 (m, 3H). |
| | 1-(4-(difluoromethoxy)phenyl)-7- | |
| | (ethylamino)-3-(2-methyl-2H- | |
| | indazol-5-yl)-3,4- | |
| | dihydropyrimido[4,5-d]pyrimidin- | |
| | 2(1H)-one | |
| Example 191 | 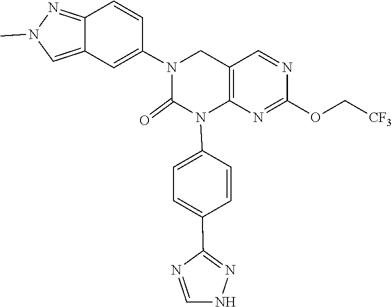 [SMILES: Cn1cc2cc(N3Cc4cnc(OCC(F)(F)F)nc4N(c4ccc(-c5nc[nH]n5)cc4)C3=O)ccc2n1] | LC-MS (ESI): m/z 522.2 [M + H]+. 1H NMR (400 MHz, DMSO-d₆) δ: 14.20 (bs, 1H), 8.62 (s, 1H), 8.39 (s, 1H), 8.38 (s, 1H), 8.12 (d, J = 8.0 Hz, 2H), 7.76 (d, J = 1.2 Hz, 1H), 7.61 (d, J = 8.8 Hz, 1H), 7.53-7.42 (m, 2H), 7.31 (dd, J = 9.2, 2.0 Hz, 1H), 5.00 (s, 2H), 4.83 (q, J_{HF} = 9.2 Hz, 2H), 4.18 (s, 3H). |
| | 1-(4-(1H-1,2,4-triazol-3- | |
| | yl)phenyl)-3-(2-methyl-2H- | |
| | indazol-5-yl)-7-(2,2,2- | |
| | trifluoroethoxy)-3,4- | |
| | dihydropyrimido[4,5-d]pyrimidin- | |
| | 2(1H)-one | |
| Example 192 | 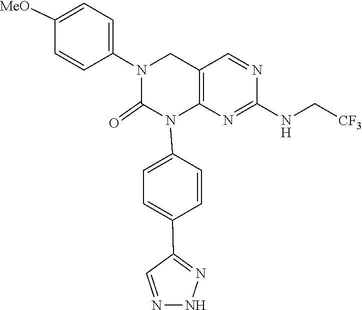 [SMILES: COc1ccc(N2Cc3cnc(NCC(F)(F)F)nc3N(c3ccc(-c4cn[nH]n4)cc3)C2=O)cc1] | LC-MS (ESI): m/z 497.2 [M + H]+. 1H NMR (400 MHz, DMSO-d₆) δ: 8.38 (s, 1H), 8.13 (s, 1H), 7.92 (d, J = 8.4 Hz, 2H), 7.49 (bs, 1H), 7.38 (d, J = 8.4 Hz, 2H), 7.35 (d, J = 8.8 Hz, 2H), 6.97 (d, J = 8.8 Hz, 2H), 4.78 (s, 2H), 4.00-3.60 (m, 2H), 3.77 (s, 3H). |
| | 1-(4-(2H-1,2,3-triazol-4- | |
| | yl)phenyl)-3-(4-methoxyphenyl)- | |
| | 7-((2,2,2-trifluoroethyl)amino)- | |
| | 3,4-dihydropyrimido[4,5- | |
| | d]pyrimidin-2(1H)-one | |
| | (synthesized from 3-(4- | |
| | methoxyphenyl)-7-(methylthio)- | |
| | 3,4-dihydropyrimido[4,5- | |
| | d]pyrimidin-2(1H)-one and 4-(4- | |
| | bromophenyl)-2-((2- | |
| | (trimethylsilyl)ethoxy)methyl)- | |
| | 2H-1,2,3-triazole (Ref: | |
| | WO2008156726 A1)) | |
| Example 193 | 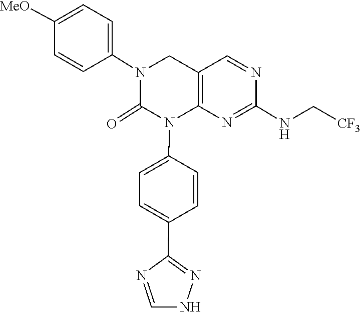 [SMILES: COc1ccc(N2Cc3cnc(NCC(F)(F)F)nc3N(c3ccc(-c4nc[nH]n4)cc3)C2=O)cc1] | LC-MS (ESI): m/z 497.0 [M + H]+. 1H NMR (400 MHz, TFA-D1) δ: 9.38 (s, 1H), 8.14 (d, J = 8.0 Hz, 2H), 8.04 (s, 1H), 7.53 (d, J = 8.4 Hz, 2H), 7.23 (dd, J = 8.8 Hz, 1.6 Hz, 2H), 7.00 (dd, J = 8.8 Hz, 1.6 Hz, 2H), 4.87 (s, 2H), 3.84 (d, J = 1.6 Hz, 3H), 3.65 (q, J = 7.6 Hz, 2H). |
| | 1-(4-(1H-1,2,4-triazol-3- | |
| | yl)phenyl)-3-(4-methoxyphenyl)- | |
| | 7-((2,2,2-trifluoroethyl)amino)- | |
| | 3,4-dihydropyrimido[4,5- | |
| | d]pyrimidin-2(1H)-one | |
| Example 194 | 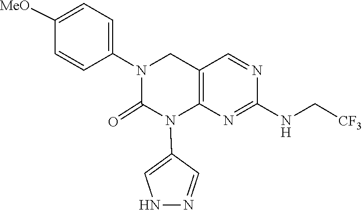 [SMILES: COc1ccc(N2Cc3cnc(NCC(F)(F)F)nc3N(c3cn[nH]c3)C2=O)cc1] | LC-MS (ESI): m/z 420.0 [M + H]+. 1H NMR (400 MHz, DMSO-d₆) δ: 12.83 (s, 1H), 8.11 (s, 1H), 7.81 (s, 1H), 7.60 (s, 1H), 7.48 (s, 1H), 7.31 (dd, J = 6.8 Hz, 2.0 Hz, 2H), 7.96 (dd, J = 6.8 Hz, 2.0 Hz, 2H), 4.71 (s, 2H), 4.05-3.83 (m, 2H), 3.77 (s, 3H). |
| | 3-(4-methoxyphenyl)-1-(1H- | |
| | pyrazol-4-yl)-7-((2,2,2- | |
| | trifluoroethyl)amino)-3,4- | |
| | dihydropyrimido[4,5-d]pyrimidin- | |
| | 2(1H)-one (synthesized from 3-(4- | |
| | methoxyphenyl)-7-(methylthio)- | |
| | 3,4-dihydropyrimido[4,5- | |
| | d]pyrimidin-2(1H)-one and 4-bromo-1-((2- | |
| | (trimethylsilyl)ethoxy)methyl)- | |
| | 1H-pyrazole (Ref: Journal of | |
| | Organic Chemistry, | |
| | 81(2), 689-698; 2016)) | |
| Example 195 | 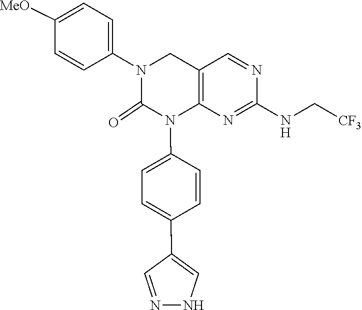 [SMILES: COc1ccc(N2Cc3cnc(NCC(F)(F)F)nc3N(c3ccc(-c4cn[nH]c4)cc3)C2=O)cc1] | LC-MS (ESI): m/z 496.0 [M + H]+. 1H NMR (400 MHz, DMSO-d₆) δ: 13.32 and 12.93 (two sets of s, 1H in totals), 8.13 (s, 1H), 7.86 (d, J = 7.6 Hz, 2H), 7.78 (s, 1H), 7.54 (bs, 1H), 7.37-7.32 (m, 4H), 6.98 (d, J = 8.8 Hz, 2H), 6.75 (d, J = 2.0 Hz, 1H), 4.78 (s, 2H), 4.11-3.60 (m, 2H), 3.77 (s, 3H). |
| | 1-(4-(1H-pyrazol-3-yl)phenyl)-3- | |
| | (4-methoxyphenyl)-7-((2,2,2- | |
| | trifluoroethyl)amino)-3,4- | |
| | dihydropyrimido[4,5-d]pyrimidin- | |
| | 2(1H)-one (synthesized from 3-(4- | |
| | methoxyphenyl)-7-(methylthio)- | |
| | 3,4-dihydropyrimido[4,5- | |
| | d]pyrimidin-2(1H)-one and 4-(4- | |
| | bromophenyl)-1-(tetrahydro-2H- | |
| | pyran-2-yl)-1H-pyrazole (Ref: | |
| | PCT Int. Appl., 2014151899, 25 | |
| | Sep 2014)) | |
| Example 196 | 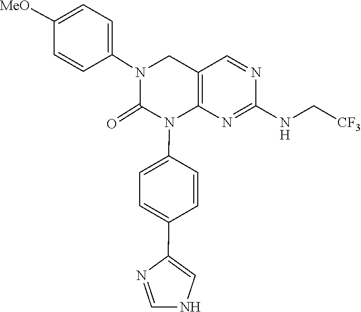 [SMILES: COc1ccc(N2Cc3cnc(NCC(F)(F)F)nc3N(c3ccc(-c4c[nH]cn4)cc3)C2=O)cc1] | LC-MS (ESI): m/z 496.2 [M + H]+. 1H NMR (400 MHz, DMSO-d₆) δ: 8.13 (d, J = 10.8 Hz, 2H), 7.81 (d, J = 8.0 Hz, 2H), 7.73 (s, 1H), 7.61 (s, 1H), 7.47 (bs, 1H), 7.34 (d, J = 8.8 Hz, 2H), 7.25 (d, J = 8.0 Hz, 2H), 6.97 (d, J = 8.8 Hz, 2H), 4.77 (s, 2H), 4.05-3.53 (m, 2H), 3.77 (s, 3H). |
| | 1-(4-(1H-imidazol-4-yl)phenyl)- | |
| | 3-(4-methoxyphenyl)-7-((2,2,2- | |
| | trifluoroethyl)amino)-3,4- | |
| | dihydropyrimido[4,5-d]pyrimidin- | |
| | 2(1H)-one (synthesized from 3-(4- | |
| | methoxyphenyl)-7-(methylthio)- | |
| | 3,4-dihydropyrimido[4,5- | |
| | d]pyrimidin-2(1H)-one and 4-(4- | |
| | bromophenyl)-1-((2- | |
| | (trimethylsilyl)ethoxy)methyl)- | |
| | 1H-imidazole (Ref: PCT Int. | |
| | Appl., 2010027500, 11 Mar 2010)) | |
| Example 197 | 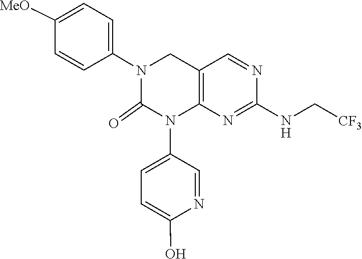 [SMILES: COc1ccc(N2Cc3cnc(NCC(F)(F)F)nc3N(c3ccc(O)nc3)C2=O)cc1] | LC-MS (ESI): m/z 447.0 [M + H]+. 1H NMR (400 MHz, DMSO-d₆) δ: 11.69 (s, 1H), 8.11 (s, 1H), 7.62 (bs, 1H), 7.42 (s, 1H), 7.37 (dd, J = 9.6 Hz, 2.8 Hz, 1H), 7.32 (d, J = 8.8 Hz, 2H), 6.97 (d, J = 8.8 Hz, 2H), 6.35 (d, J = 9.6 Hz, 1H), 4.72 (s, 2H), 4.10-3.67 (m, 2H), 3.77 (s, 3H). |
| | 1-(6-hydroxypyridin-3-yl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one (synthesized from 3-(4- | |
| | methoxyphenyl)-7-(methylthio)- | |
| | 3,4-dihydropyrimido[4,5- | |
| | d]pyrimidin-2(1H)-one and 5-bromo-1-((2- | |
| | (trimethylsilyl)ethoxy)methyl)pyridin- | |
| | 2(1H)-one (Ref: PCT Int. | |
| | Appl., 2015066188, 07 May | |
| | 2015)) | |
| Example 419 | 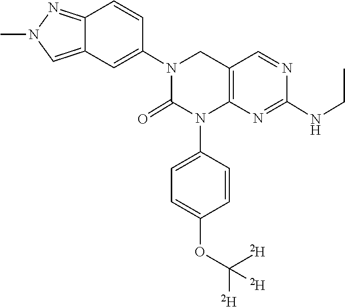 [SMILES: [2H]C([2H])([2H])Oc1ccc(N2C(=O)N(c3ccc4nn(C)cc4c3)Cc3cnc(NCC)nc32)cc1] | LC-MS: m/z 433 (M + H)+. 1H NMR (400 MHz, DMSO-d₆) δ: 8.34 (s, 1H), 8.03 (s, 1H), 7.68 (d, J = 1.6 Hz, 1H), 7.57 (d, J = 9.2 Hz, 1H), 7.25 (dd, J = 9.2 Hz, 2.0 Hz, 1H), 7.22-7.15 (m, 2H), 7.01-6.93 (m, 2H), 6.80 (s, 1H), 4.78 (s, 2H), 4.16 (s, 3H), 3.08 (s, 2H), 0.99 (s, 3H). |
| | 7-(ethylamino)-1-(4-(methoxy- | |
| | d3)phenyl)-3-(2-methyl-2H- | |
| | indazol-5-yl)-3,4- | |
| | dihydropyrimido[4,5-d]pyrimidin- | |
| | 2(1H)-one | |
| Example 420 | 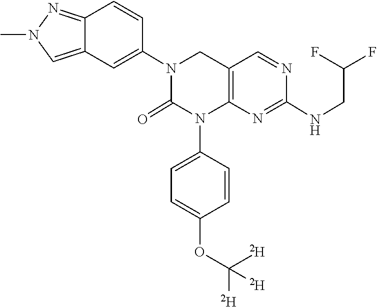 [SMILES: [2H]C([2H])([2H])Oc1ccc(N2C(=O)N(c3ccc4nn(C)cc4c3)Cc3cnc(NCC(F)F)nc32)cc1] | LC-MS: m/z 469 (M + H)+. 1H NMR (400 MHz, DMSO-d₆) δ: 8.35 (s, 1H), 8.09 (s, 1H), 7.69 (d, J = 1.5 Hz, 1H), 7.57 (d, J = 9.2 Hz, 1H), 7.26 (dd, J = 9.2 Hz, 2.0 Hz, 2H), 7.22 (d, J = 8.8 Hz, 2H), 7.02- 6.94 (m, 2H), 5.85 (br, 1H), 4.82 (s, 2H), 4.17 (s, 3H), 3.55 (s, 2H). |
| | 7-((2,2-difluoroethyl)amino)-1-(4- | |
| | (methoxy-d3)phenyl)-3-(2- | |
| | methyl-2H-indazol-5-yl)-3,4- | |
| | dihydropyrimido[4,5-d]pyrimidin- | |
| | 2(1H)-one | |

The procedure set forth above for General Procedure IV (Method B) was used to synthesize the following compounds by using appropriate starting materials:

| Cpd No. | Structure | Characterization |
|---|---|---|
| Example 198 | 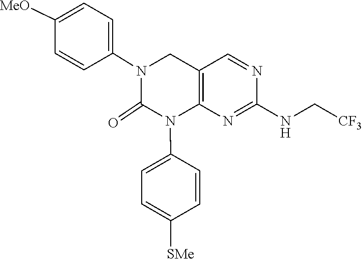 [SMILES: COc1ccc(N2Cc3cnc(NCC(F)(F)F)nc3N(c3ccc(SC)cc3)C2=O)cc1] | LC-MS (ESI): m/z 476.1 [M + H]+. 1H NMR (400 MHz, CDCl3) δ: 7.99 (s, 1H), 7.32 (d, J = 8.4 Hz, 2H), 7.28 (d, J = 8.8 Hz, 2H), 7.20 (d, J = 8.8 Hz, 2H), 6.92 (d, J = 8.8 Hz, 2H), 5.19 (s, 1H), 4.75 (s, 2H), 4.15-3.83 (m, 2H), 3.81 (s, 3H), 2.51 (s, 3H). |
| | 3-(4-methoxyphenyl)-1-(4- | |
| | (methylthio)phenyl)-7-((2,2,2- | |
| | trifluoroethyl)amino)-3,4- | |
| | dihydropyrimido[4,5-d]pyrimidin- | |
| | 2(1H)-one | |
| Example 199 | 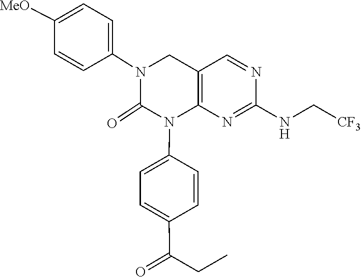 [SMILES: CCC(=O)c1ccc(N2C(=O)N(c3ccc(OC)cc3)Cc3cnc(NCC(F)(F)F)nc32)cc1] | LC-MS (ESI): m/z 486.0 [M + H]+. 1H NMR (400 MHz, DMSO-d₆) δ: 8.14 (s, 1H), 8.04 (d, J = 8.0 Hz, 2H), 7.45 (bs, 1H), 7.47 (d, J = 8.0 Hz, 2H), 7.35 (d, J = 8.8 Hz, 2H), 6.98 (d, J = 8.8 Hz, 2H), 4.79 (s, 2H), 4.15-3.62 (m, 2H), 3.77 (s, 3H), 3.09 (q, J = 7.2 Hz, 2H), 1.12 (t, J = 7.2 Hz, 3H). |
| | 3-(4-methoxyphenyl)-1-(4- | |
| | propionylphenyl)-7-((2,2,2- | |
| | trifluoroethyl)amino)-3,4- | |
| | dihydropyrimido[4,5-d]pyrimidin- | |
| | 2(1H)-one | |
| Example 200 | 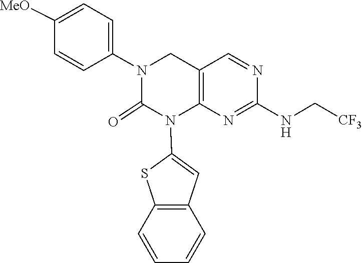 [SMILES: COc1ccc(N2Cc3cnc(NCC(F)(F)F)nc3N(c3cc4ccccc4s3)C2=O)cc1] | LC-MS (ESI): m/z 486.0 [M + H]+. 1H NMR (400 MHz, DMSO-d₆) δ: 8.18 (s, 1H), 7.95-7.92 (m, 1H), 7.86-7.83 (m, 1H), 7.63 (bs, 1H), 7.40-7.37 (m, 3H), 7.36 (d, J = 9.2 Hz, 2H), 6.98 (d, J = 8.8 Hz, 2H), 4.78 (s, 2H), 4.15-3.72 (m, 2H), 3.77 (s, 3H). |
| | 1-(benzo[b]thiophen-2-yl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 201 | 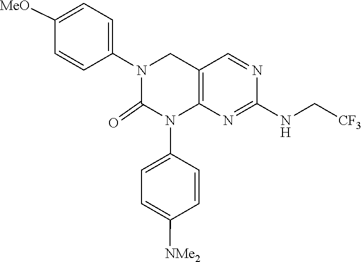 [SMILES: COc1ccc(N2Cc3cnc(NCC(F)(F)F)nc3N(c3ccc(N(C)C)cc3)C2=O)cc1] | LC-MS (ESI): m/z 473.2 [M + H]+. 1H NMR (400 MHz, CDCl3) δ: 7.96 (s, 1H), 7.29 (d, J = 9.2 Hz, 2H), 7.12 (d, J = 8.8 Hz, 2H), 6.91 (d, J = 9.2 Hz, 2H), 6.76 (d, J = 9.2 Hz, 2H), 5.20 (t, J = 6.8 Hz, 1H), 4.74 (s, 2H), 4.01-3.85 (m, 2H), 3.81 (s, 3H), 2.98 (s, 6H). |
| | 1-(4-(dimethylamino)phenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 202 | 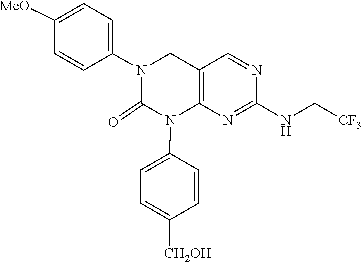 [SMILES: COc1ccc(N2Cc3cnc(NCC(F)(F)F)nc3N(c3ccc(CO)cc3)C2=O)cc1] | LC-MS (ESI): m/z 460.2 [M + H]+. 1H NMR (400 MHz, DMSO-d₆) δ: 8.12 (s, 1H), 7.47 (bs, 1H), 7.37 (d, J = 8.4 Hz, 2H), 7.33 (d, J = 8.8 Hz, 2H), 7.23 (d, J = 8.0 Hz, 2H), 6.97 (d, J = 9.2 Hz, 2H), 5.31 (t, J = 5.2 Hz, 1H), 4.76 (s, 2H), 4.54 (d, J = 5.2 Hz, 2H), 4.05- 3.85 (m, 2H), 3.77 (s, 3H). |
| | 1-(4-(hydroxymethyl)phenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 203 | 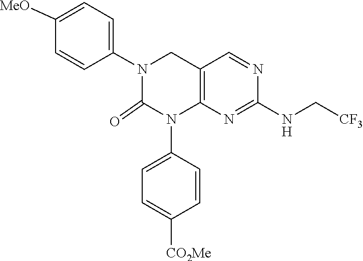 [SMILES: COC(=O)c1ccc(N2C(=O)N(c3ccc(OC)cc3)Cc3cnc(NCC(F)(F)F)nc32)cc1] | LC-MS (ESI): m/z 488.1 [M + H]+. 1H NMR (400 MHz, DMSO-d₆) δ: 8.14 (s, 1H), 8.03 (d, J = 8.4 Hz, 2H), 7.52 (bs, 1H), 7.48 (d, J = 8.4 Hz, 2H), 7.35 (d, J = 9.2 Hz, 2H), 6.98 (d, J = 8.8 Hz, 2H), 4.78 (s, 2H), 3.89 (s, 3H), 3.77 (s, 3H), 4.00-3.50 (m, 2H). |
| | methyl 4-(3-(4-methoxyphenyl)-2- | |
| | oxo-7-((2,2,2- | |
| | trifluoroethyl)amino)-3,4- | |
| | dihydropyrimido[4,5-d]pyrimidin- | |
| | 1(2H)-yl)benzoate | |
| Example 204 | 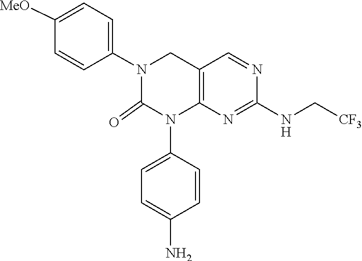 [SMILES: COc1ccc(N2Cc3cnc(NCC(F)(F)F)nc3N(c3ccc(N)cc3)C2=O)cc1] | LC-MS (ESI): m/z 445.0 [M + H]+. 1H NMR (400 MHz, DMSO-d₆) δ: 8.12 (s, 1H), 7.51 (bs, 1H), 7.37 (d, J = 8.8 Hz, 2H), 7.01 (d, J = 8.8 Hz, 2H), 6.92 (d, J = 8.4 Hz, 2H), 6.63 (d, J = 8.4 Hz, 2H), 5.19 (bs, 2H), 4.77 (s, 2H), 4.15-3.78 (m, 2H), 3.82 (s, 3H). |
| | 1-(4-aminophenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 205 | 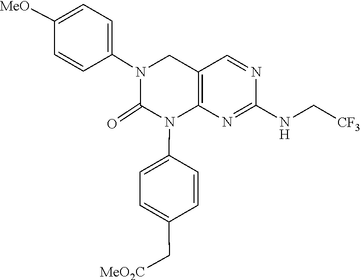 [SMILES: COC(=O)Cc1ccc(N2C(=O)N(c3ccc(OC)cc3)Cc3cnc(NCC(F)(F)F)nc32)cc1] | LC-MS (ESI): m/z 502.0 [M + H]+. 1H NMR (400 MHz, DMSO-d₆) δ: 8.12 (s, 1H), 7.48 (bs, 1H), 7.34 (d, J = 8.8 Hz, 4H), 7.24 (d, J = 8.0 Hz, 2H), 6.97 (d, J = 8.8 Hz, 2H), 4.76 (s, 2H), 4.14-3.80 (m, 2H), 3.77 (s, 3H), 3.74 (s, 2H), 3.64 (s, 3H). |
| | methyl 2-(4-(3-(4-methoxyphenyl)- | |
| | 2-oxo-7-((2,2,2- | |
| | trifluoroethyl)amino)-3,4- | |
| | dihydropyrimido[4,5-d]pyrimidin- | |
| | 1(2H)-yl)phenyl)acetate | |
| Example 206 | 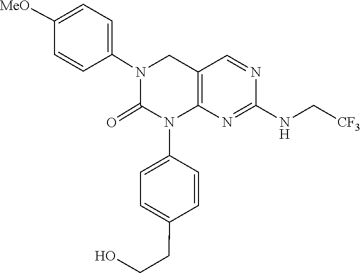 [SMILES: COc1ccc(N2Cc3cnc(NCC(F)(F)F)nc3N(c3ccc(CCO)cc3)C2=O)cc1] | LC-MS (ESI): m/z 474.0 [M + H]+. 1H NMR (400 MHz, DMSO-d₆) δ: 8.10 (s, 1H), 7.45 (bs, 1H), 7.33 (d, J = 8.8 Hz, 2H), 7.27 (d, J = 8.0 Hz, 2H), 7.17 (d, J = 8.4 Hz, 2H), 6.96 (d, J = 9.2 Hz, 2H), 4.75 (s, 2H), 4.70 (s, 1H), 4.12-3.78 (m, 2H), 3.76 (s, 3H), 3.64 (s, 2H), 2.77 (t, J = 7.2 Hz, 2H). |
| | 1-(4-(2-hydroxyethyl)phenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 207 | 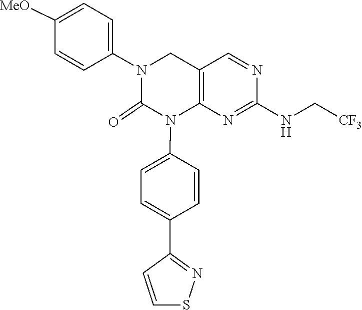 [SMILES: COc1ccc(N2Cc3cnc(NCC(F)(F)F)nc3N(c3ccc(-c4ccsn4)cc3)C2=O)cc1] | LC-MS (ESI): m/z 513.1 [M + H]+. 1H NMR (400 MHz, CDCl3-d) δ: 8.42 (d, J = 1.6 Hz, 1H), 7.96 (s, 1H), 7.62 (d, J = 8.4 Hz, 2H), 7.37 (d, J = 1.6 Hz, 1H), 7.32 (d, J = 8.4 Hz, 2H), 7.23 (d, J = 8.8 Hz, 2H), 6.87 (d, J = 8.8 Hz, 2H), 5.11 (bs, 1H), 4.71 (s, 2H), 3.95-3.57 (m, 2H), 3.75 (s, 3H). |
| | 1-(4-(isothiazol-3-yl)phenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 208 | 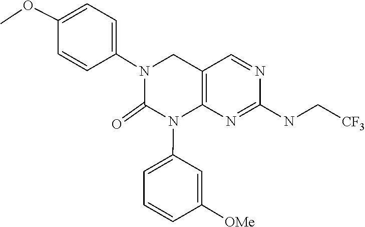 | LC-MS: m/z 460.2 (M + H)+. 1H NMR (400 MHz, DMSO-d₆) δ: 8.10 (s, 1H), 7.50 (br s, 1H), 7.37- 7.29 (m, 3H), 6.99-6.92 (m, 3H), 6.90-6.83 (m, 2H), 4.75 (s, 2H), 4.06-3.67 (m, 2H), 3.76 (s, 3H), 3.75 (s, 3H). |
| | 1-(3-methoxyphenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 209 | 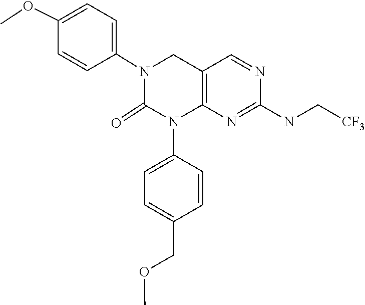 | LC-MS: m/z 474.2 (M + H)+. 1H NMR (400 MHz, DMSO-d₆) δ: 8.10 (s, 1H), 7.50 (br s, 1H), 7.37 (d, J = 8.4 Hz, 2H), 7.33 (d, J = 8.8 Hz, 2H), 7.26 (d, J = 7.6 Hz, 2H), 6.96 (d, J = 8.8 Hz, 2H), 4.76 (s, 2H), 4.45 (s, 2H), 4.03-3.60 (m, 2H), 3.76 (s, 3H), 3.32 (s, 3H). |
| | 1-(4-(methoxymethyl)phenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 210 | 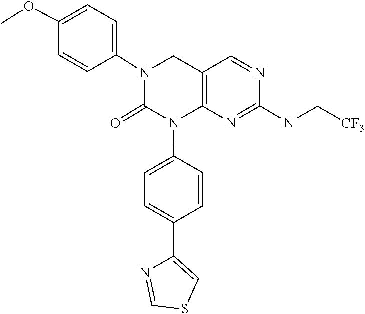 | LC-MS: m/z 513.1 (M + H)+. 1H NMR (400 MHz, DMSO-d₆) δ: 9.22 (d, J = 1.6 Hz, 1H), 8.23 (d, J = 2.0 Hz, 1H), 8.13 (s, 1H), 8.05 (d, J = 8.8 Hz, 2H), 7.49 (br s, 1H), 7.45-7.32 (m, 4H), 6.97 (d, J = 8.8 Hz, 2H), 4.78 (s, 2H), 4.05-3.60 (m, 2H), 3.77 (s, 3H). |
| | 3-(4-methoxyphenyl)-1-(4-(thiazol- | |
| | 4-yl)phenyl)-7-((2,2,2- | |
| | trifluoroethyl)amino)-3,4- | |
| | dihydropyrimido[4,5-d]pyrimidin- | |
| | 2(1H)-one | |
| Example 211 | 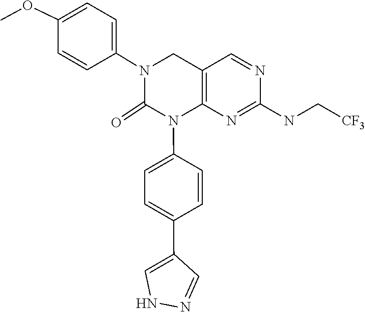 | LC-MS: m/z 496.2 (M + H)+. 1H NMR (400 MHz, DMSO-d₆) δ: 12.97 (br s, 1H), 8.35-7.88 (m, 2H), 8.12 (s, 1H), 7.66 (d, J = 8.8 Hz, 2H), 7.50 (br s, 1H), 7.34 (d, J = 8.8 Hz, 2H), 7.26 (d, J = 8.4 Hz, 2H), 6.97 (d, J = 8.8 Hz, 2H), 4.77 (s, 2H), 4.09-3.65 (m, 2H), 3.77 (s, 3H). |
| | 1-(4-(1H-pyrazol-4-yl)phenyl)-3- | |
| | (4-methoxyphenyl)-7-((2,2,2- | |
| | trifluoroethyl)amino)-3,4- | |
| | dihydropyrimido[4,5-d]pyrimidin- | |
| | 2(1H)-one | |
| Example 212 | 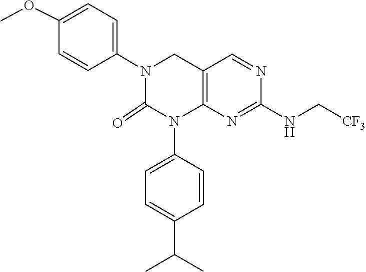 [SMILES: COc1ccc(N2Cc3cnc(NCC(F)(F)F)nc3N(c3ccc(C(C)C)cc3)C2=O)cc1] | LC-MS: m/z 472.2 (M + H)+. 1H NMR (400 MHz, DMSO-d₆) δ: 8.10 (s, 1H), 7.50 (br s, 1H), 7.33 (d, J = 9.2 Hz, 1H), 7.30 (d, J = 8.0 Hz, 2H), 7.18 (d, J = 8.0 Hz, 2H), 6.96 (d, J = 8.8 Hz, 2H), 4.76 (s, 2H), 4.13-3.53 (m, 2H), 3.70 (s, 3H), 2.94 (hept, J = 6.8 Hz, 1H), 1.23 (d, J = 6.8 Hz, 6H). |
| | 1-(4-isopropylphenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 213 | 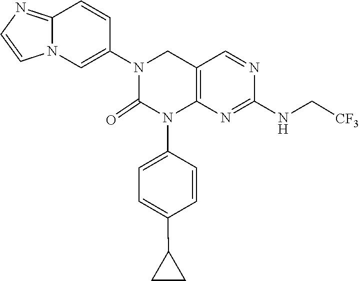 [SMILES: O=C1N(c2ccc3nccn3c2)Cc2cnc(NCC(F)(F)F)nc2N1c1ccc(C2CC2)cc1] | LC-MS: m/z 480.2 (M + H)+. 1H NMR (400 MHz, DMSO-d₆) δ: 8.74 (s, 1H), 8.15 (s, 1H), 8.12 (s, 1H), 7.96 (s, 1H), 7.62-7.54 (m, 2H), 7.35 (dd, J = 9.6 Hz, 2.0 Hz, 1H), 7.16 (d, J = 8.8 Hz, 2H), 7.13 (d, J = 8.8 Hz, 2H), 4.84 (s, 2H), 4.05-3.60 (m, 2H), 1.98-1.90 (m, 1H), 1.00-0.95 (m, 2H), 0.71-0.65 (m, 2H). |
| | 1-(4-cyclopropylphenyl)-3- | |
| | (imidazo[1,2-a]pyridin-6-yl)-7- | |
| | ((2,2,2-trifluoroethyl)amino)-3,4- | |
| | dihydropyrimido[4,5-d]pyrimidin- | |
| | 2(1H)-one | |

### General Procedure V

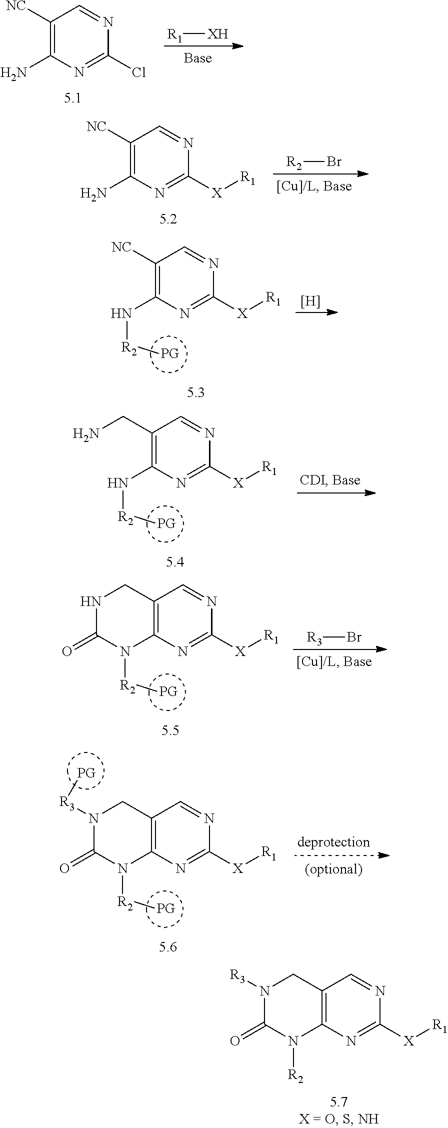

Compounds of structure 5.7 were obtained through the scheme depicted as General Procedure V. Beginning with aryl-chloride 5.1, the desired R₁ group was introduced using a base mediated aromatic substitution reaction to generate 5.2. The desired R² group (optionally protected) was introduced by a copper mediated C—N coupling reaction with urea 5.2 to generate compound 5.3. Nitrile 5.3 was reduced to amine 5.4 using hydrogenation conditions. Diamine 5.4 was converted to cyclic urea 5.5 using CDI. The desired R₃ group (optionally protected) was introduced by a copper mediated C—N coupling reaction with urea 5.5 to generate compound 5.6. If necessary, compound 5.6 was deprotected to afford compound 5.7.

### Preparation of Example 214 Via General Procedure V

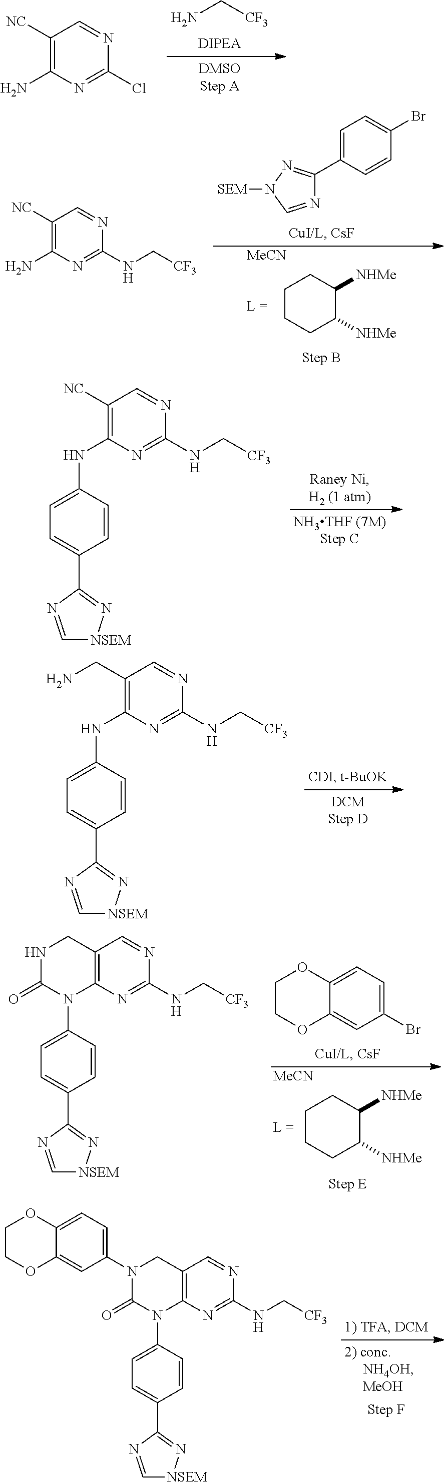

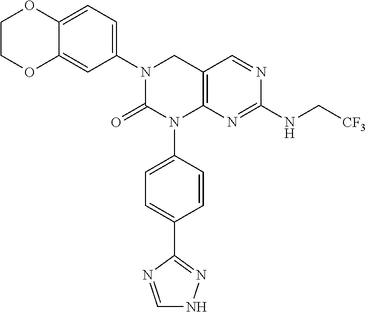 [SMILES: O=C1N(c2ccc3c(c2)OCCO3)Cc2cnc(NCC(F)(F)F)nc2N1c1ccc(-c2nc[nH]n2)cc1]

### Step A: 4-amino-2-((2,2,2-trifluoroethyl)amino)pyrimidine-5-carbonitrile

To a solution of 4-amino-2-chloropyrimidine-5-carbonitrile (1.0 g, 6.5 mmol, 1.0 eq.) in DMSO (10 mL) was added 2,2,2-trifluoroethan-1-amine (1.9 g, 19.5 mmol, 3.0 eq.) and DIPEA (2.5 g, 19.5 mmol, 3.0 eq.) at room temperature, and the reaction mixture was stirred at 80° C. for 2 hrs in a sealed tube. Then the reaction mixture was quenched with ice water (30 mL) and extracted with EtOAc (50 mL×3). The combined organic layers were washed with brine (50 mL), dried over Na₂SO₄ and concentrated under reduced pressure, the residue was purified by flash column chromatography silica gel to give 4-amino-2-((2,2,2-trifluoroethyl)amino)pyrimidine-5-carbonitrile (1.3 g, 93% yield) as a white solid. LC-MS (ESI): m/z 218 [M+H]⁺.

### Step B: 2-((2,2,2-trifluoroethyl)amino)-4-((4-(1-((2-(trimethylsilyl)ethoxy)methyl)-1H-1,2,4-triazol-3-yl)phenyl)amino)pyrimidine-5-carbonitrile

To a solution of 4-amino-2-((2,2,2-trifluoroethyl)amino)pyrimidine-5-carbonitrile (1.1 g, 5.1 mmol, 1.0 eq.) in MeCN (10 mL) was added CuI (960 mg, 5.1 mmol, 1.0 eq.), CsF (2.3 g, 15.3 mmol, 3.0 eq.), N¹,N²-dimethylcyclohexane-1,2-diamine (1.1 g, 7.7 mmol, 1.5 eq.) and 3-(4-bromophenyl)-1-((2-(trimethylsilyl)ethoxy)methyl)-1H-1,2,4-triazole (Ref: WO2008156726 A1) (2.7 g, 7.7 mmol, 1.5 eq.) at room temperature, and the resulting mixture was stirred at 100° C. for additional 16 hrs. Then the reaction mixture was quenched with ice water (30 mL) and extracted with EtOAc (50 mL×3). The combined organic layers were washed with brine (50 mL), dried over Na₂SO₄ and concentrated under reduced pressure, the residue was purified by flash column chromatography on silica gel to give 2-((2,2,2-trifluoroethyl)amino)-4-((4-(1-((2-(trimethylsilyl)ethoxy)methyl)-1H-1,2,4-triazol-3-yl)phenyl)amino)pyrimidine-5-carbonitrile (1.8 g, 72% yield) as a white solid. LC-MS (ESI): m/z 491 [M+H]⁺.

### Step C: 5-(aminomethyl)-N₂-(2,2,2-trifluoroethyl)-N₄-(4-(1-((2-(trimethylsilyl)ethoxy)methyl)-1H-1,2,4-triazol-3-yl)phenyl)pyrimidine-2,4-diamine

To a solution of 2-((2,2,2-trifluoroethyl)amino)-4-((4-(1-((2-(trimethylsilyl)ethoxy)methyl)-1H-1,2,4-triazol-3-yl)phenyl)amino)pyrimidine-5-carbonitrile (800 mg, 1.6 mmol, 1.0 eq.) in NH₃-THF (7 mol/L, 10 mL, 7.0 mmol, 4.4 eq.) was added Raney Ni (180 mg) at room temperature. The reaction mixture was stirred at room temperature for 16 hrs with H₂ balloon (1 atm). After completion, the catalyst was removed by filtration, and the filtrate was concentrated under reduced pressure to give crude 5-(aminomethyl)-N₂-(2,2,2-trifluoroethyl)-N₄-(4-(1-((2-(trimethylsilyl)ethoxy)methyl)-1H-1,2,4-triazol-3-yl)phenyl)pyrimidine-2,4-diamine (800 mg) as a white solid, which was used in next step without further purification. LC-MS (ESI): m/z 495 [M+H]⁺.

### Step D: 7-((2,2,2-trifluoroethyl)amino)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one

To a solution of 5-(aminomethyl)-2-(methylthio)-N-(4-(1-((2-(trimethylsilyl)ethoxy)methyl)-1H-1,2,4-triazol-3-yl)phenyl)pyrimidin-4-amine (800 mg, 1.6 mmol, 1.0 eq.) in DCM (10 mL) was added CDI (520 mg, 3.2 mmol, 2.0 eq.) and t-BuOK (718 mg, 6.4 mmol, 4.0 eq.) at room temperature. And the reaction mixture was stirred at room temperature for 16 h. Then the reaction mixture was quenched with ice water (30 mL) and extracted with DCM (30 mL×3). The combined organic layers were washed with brine (50 mL), dried over Na₂SO₄ and concentrated under reduced pressure. The residue was purified by flash column chromatography on silica gel to give 7-((2,2,2-trifluoroethyl)amino)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (750 mg, 90% yield) as a white solid. LC-MS (ESI): m/z 521 [M+H]⁺.

### Step E: 3-(2,3-dihydrobenzo[b][1,4]dioxin-6-yl)-7-((2,2,2-trifluoroethyl)amino)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one

To a solution of 7-((2,2,2-trifluoroethyl)amino)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (50 mg, 0.1 mmol, 1.0 eq.) in MeCN (2 mL) was added CuI (20 mg, 0.1 mmol, 1.0 eq.), CsF (46 mg, 0.3 mmol, 3.0 eq.), N¹,N²-dimethylcyclohexane-1,2-diamine (21 mg, 0.15 mmol, 1.5 eq.) and 6-bromo-2,3-dihydrobenzo[b][1,4]dioxine (32 mg, 0.15 mmol, 1.5 eq.) at room temperature, and the resulting mixture was stirred at 90° C. for 16 hrs. Then the reaction mixture was quenched with ice water (30 mL) and extracted with EtOAc (30 mL×3). The combined organic layers were washed with brine (30 mL), dried over Na₂SO₄ and concentrated under reduced pressure, the residue was purified by flash column chromatography on silica gel to give 3-(2,3-dihydrobenzo[b][1,4]dioxin-6-yl)-7-((2,2,2-trifluoroethyl)amino)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (50 mg, 77% yield) as a white solid. LC-MS (ESI): m/z 655 [M+H]⁺.

### Step F: 1-(4-(1H-1,2,4-triazol-3-yl)phenyl)-3-(2,3-dihydrobenzo[b][1,4]dioxin-6-yl)-7-((2,2,2-trifluoroethyl)amino)-3,4-dihydropyrimido[4,5-d]pyrimidin-2(1H)-one

To a solution of 3-(2,3-dihydrobenzo[b][1,4]dioxin-6-yl)-7-((2,2,2-trifluoroethyl)amino)-1-(4-(1-((2-(trimethylsilyl)ethoxy)methyl)-1H-1,2,4-triazol-3-yl)phenyl)-3,4-dihydropyrimido[4,5-d]pyrimidin-2(1H)-one (50 mg, 0.08 mmol, 1.0 eq.) in DCM (3 mL) was added TFA (0.5 mL) at room temperature. The resulting mixture was stirred for additional 16 hrs. Most of solvents were evaporated under reduced pressure, the residue was diluted with MeOH (1 mL), added conc. NH₄OH (0.5 mL) and stirred for 2 hrs at room temperature. Then the reaction mixture was concentrated under reduced pressure. The crude residue was purified by RP-prep-HPLC to afford 1-(4-(1H-1,2,4-triazol-3-yl)phenyl)-3-(2,3-dihydrobenzo[b][1,4]dioxin-6-yl)-7-((2,2,2-trifluoroethyl)amino)-3,4-dihydropyrimido[4,5-d]pyrimidin-2(1H)-one (Example 214).

¹H NMR (400 MHz, DMSO-d₆) δ (ppm): 14.16 (s, 1H), 8.46 (s, 1H), 8.12 (s, 1H), 8.07 (d, J=8.4 Hz, 2H), 7.53-7.41 (m, 3H), 6.97 (s, 1H), 6.89 (s, 2H), 4.76 (s, 2H), 4.26 (s, 4H), 4.10-3.52 (m, 2H).

LC-MS (ESI): m/z 525 [M+H]⁺.

The procedure set forth above for General Procedure V was used to synthesize the following compounds by using appropriate starting materials:

| Cpd No. | Structure | Characterization |
|---|---|---|
| Example 215 | 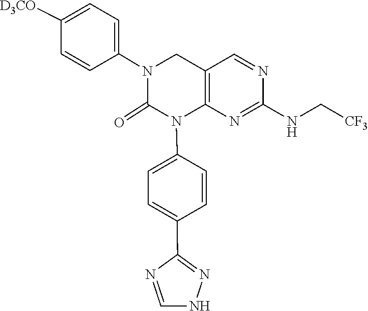 | LC-MS (ESI): m/z 500.0 [M + H]+. 1H NMR (400 MHz, DMSO-d₆) δ: 8.41 (s, 1H), 8.08 (s, 1H), 8.05 (d, J = 8.4 Hz, 2H), 7.38 (d, J = 8.0 Hz, 2H), 7.31 (d, J = 8.8 Hz, 2H), 6.95 (d, J = 8.2 Hz, 2H), 4.74 (s, 2H), 3.86-3.58 (m, 2H). |
| | 1-(4-(1H-1,2,4-triazol-3-yl)phenyl)- | |
| | 3-(4-(methoxy-d3)phenyl)-7-((2,2,2- | |
| | trifluoroethyl)amino)-3,4- | |
| | dihydropyrimido[4,5-d]pyrimidin- | |
| | 2(1H)-one | |
| Example 216 | 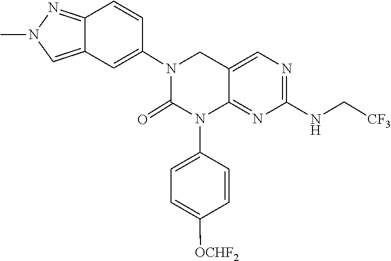 [SMILES: Cn1cc2cc(N3Cc4cnc(NCC(F)(F)F)nc4N(c4ccc(OC(F)F)cc4)C3=O)ccc2n1] | LC-MS (ESI): m/z 520.1 [M + H]+. 1H NMR (400 MHz, DMSO-d₆) δ: 8.36 (s, 1H), 8.14 (s, 1H), 7.71 (d, J = 1.2 Hz, 1H), 7.58 (d, J = 9.2 Hz, 1H), 7.45 (bs, 1H), 7.38 (d, J = 8.4 Hz, 2H), 7.30 (t, J_{HF} = 74.4 Hz, 1H), 7.28 (dd, J = 9.2 Hz, 2.0 Hz, 1H), 7.24 (d, J = 8.8 Hz, 2H), 4.84 (s, 2H), 4.17 (s, 3H), 4.08-3.57 (m, 2H). |
| | 1-(4-(difluoromethoxy)phenyl)-3-(2- | |
| | methyl-2H-indazol-5-yl)-7-((2,2,2- | |
| | trifluoroethyl)amino)-3,4- | |
| | dihydropyrimido[4,5-d]pyrimidin- | |
| | 2(1H)-one | |
| Example 217 | 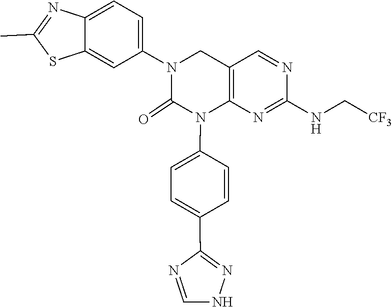 [SMILES: Cc1nc2ccc(N3Cc4cnc(NCC(F)(F)F)nc4N(c4ccc(-c5nc[nH]n5)cc4)C3=O)cc2s1] | LC-MS (ESI): m/z 538.0 [M + H]+. 1H NMR (400 MHz, DMSO-d₆) δ: 14.20 (bs, 1H), 8.60 (bs, 1H), 8.16 (s, 1H), 8.13 (d, J = 2.0 Hz, 1H), 8.08 (d, J = 8.4 Hz, 2H), 7.93 (d, J = 8.8 Hz, 1H), 7.56 (dd, J = 8.8 Hz, 2.4 Hz, 1H), 7.50-7.38 (m, 3H), 4.91 (s, 2H), 4.08-3.55 (m, 2H), 2.81 (s, 3H). |
| | 1-(4-(1H-1,2,4-triazol-3-yl)phenyl)- | |
| | 3-(2-methylbenzo[d]thiazol-6-yl)-7- | |
| | ((2,2,2-trifluoroethyl)amino)-3,4- | |
| | dihydropyrimido[4,5-d]pyrimidin- | |
| | 2(1H)-one | |
| Example 218 | 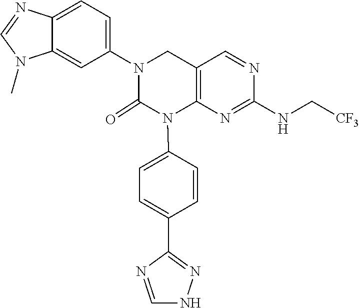 [SMILES: Cn1cnc2ccc(N3Cc4cnc(NCC(F)(F)F)nc4N(c4ccc(-c5nc[nH]n5)cc4)C3=O)cc21] | LC-MS (ESI): m/z 521.0 [M + H]+. 1H NMR (400 MHz, DMSO-d₆) δ: 14.50 and 14.13 (two sets of s, 1H in total), 8.64 and 8.08 (two sets of s, 1H in total), 8.23 (s, 1H), 8.16 (s, 1H), 8.10-8.06 (m, 2H), 7.69-7.65 (m, 2H), 7.52 (bs, 1H), 7.50 (d, J = 8.0 Hz, 1H), 7.40 (d, J = 8.4 Hz, 1H), 7.29 (dd, J = 8.4 Hz, 2.0 Hz, 1H), 4.89 (s, 2H), 4.10-3.68 (m, 2H), 3.84 (s, 3H). |
| | 1-(4-(1H-1,2,4-triazol-3-yl)phenyl)- | |
| | 3-(1-methyl-1H-benzo[d]imidazol-6- | |
| | yl)-7-((2,2,2-trifluoroethyl)amino)- | |
| | 3,4-dihydropyrimido[4,5- | |
| | d]pyrimidin-2(1H)-one | |
| Example 219 | 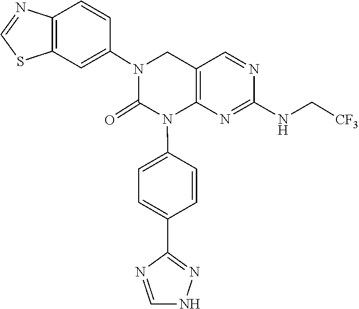 [SMILES: O=C1N(c2ccc3ncsc3c2)Cc2cnc(NCC(F)(F)F)nc2N1c1ccc(-c2nc[nH]n2)cc1] | LC-MS (ESI): m/z 524.1 [M + H]+. 1H NMR (400 MHz, DMSO-d₆) δ: 9.41 (s, 1H), 8.46 (s, 1H), 8.27 (d, J = 2.0 Hz, 1H), 8.17 (s, 1H), 8.11 (d, J = 8.8 Hz, 1H), 8.09 (d, J = 8.4 Hz, 2H), 7.65 (dd, J = 8.8, 2.0 Hz, 1H), 7.54 (bs, 1H), 7.46 (d, J = 8.4 Hz, 2H), 4.93 (s, 2H), 3.94-3.70 (m, 2H). |
| | 1-(4-(1H-1,2,4-triazol-3-yl)phenyl)- | |
| | 3-(benzo[d]thiazol-6-yl)-7-((2,2,2- | |
| | trifluoroethyl)amino)-3,4- | |
| | dihydropyrimido[4,5-d]pyrimidin- | |
| | 2(1H)-one | |
| Example 220 | 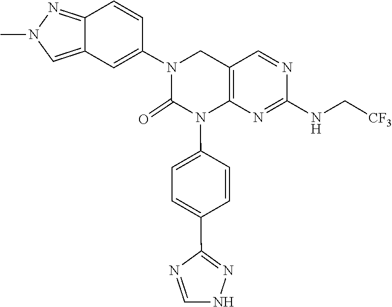 [SMILES: Cn1cc2cc(N3Cc4cnc(NCC(F)(F)F)nc4N(c4ccc(-c5nc[nH]n5)cc4)C3=O)ccc2n1] | LC-MS (ESI): m/z 521.2 [M + H]+. 1H NMR (400 MHz, DMSO-d₆) δ: 14.19 (bs, 1H), 8.62-8.40 (m, 1H), 8.37 (s, 1H), 8.16 (s, 1H), 8.08 (d, J = 8.4 Hz, 2H), 7.73 (d, J = 1.6 Hz, 1H), 7.59 (d, J = 9.2 Hz, 1H), 7.46 (bs, 1H), 7.44 (d, J = 7.6 Hz, 2H), 7.29 (dd, J = 9.2 Hz, 1.6 Hz, 1H), 4.87 (s, 2H), 4.18 (s, 3H), 4.03-3.53 (m, 2H). |
| | 1-(4-(1H-1,2,4-triazol-3-yl)phenyl)- | |
| | 3-(2-methyl-2H-indazol-5-yl)-7- | |
| | ((2,2,2-trifluoroethyl)amino)-3,4- | |
| | dihydropyrimido[4,5-d]pyrimidin- | |
| | 2(1H)-one | |
| Example 221 | 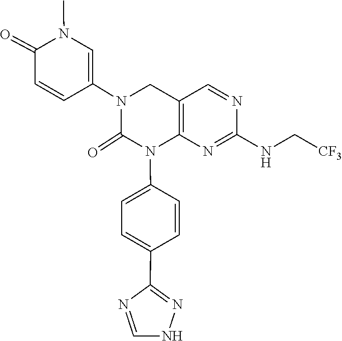 [SMILES: Cn1cc(N2Cc3cnc(NCC(F)(F)F)nc3N(c3ccc(-c4nc[nH]n4)cc3)C2=O)ccc1=O] | LC-MS (ESI): m/z 498.2 [M + H]+. 1H NMR (400 MHz, DMSO-d₆) δ: 8.43 (s, 1H), 8.13 (s, 1H), 8.07 (d, J = 8.4 Hz, 2H), 7.93 (d, J = 2.8 Hz, 1H), 7.56 (dd, J = 9.6, 2.8 Hz, 1H), 7.50 (bs, 1H), 7.38 (d, J = 8.0 Hz, 2H), 6.42 (d, J = 9.6 Hz, 1H), 4.73 (s, 2H), 4.00-3.50 (m, 2H), 3.44 (s, 3H). |
| | 1-(4-(1H-1,2,4-triazol-3-yl)phenyl)- | |
| | 3-(1-methyl-6-oxo-1 | |
| | dihydropyridin-3-yl)-7-((2,2,2- | |
| | trifluoroethyl)amino)-3,4- | |
| | dihydropyrimido[4,5-d]pyrimidin- | |
| | 2(1H)-one | |
| Example 222 | 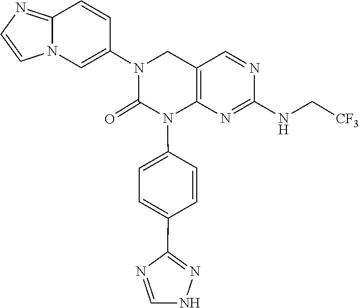 [SMILES: O=C1N(c2ccc3nccn3c2)Cc2cnc(NCC(F)(F)F)nc2N1c1ccc(-c2nc[nH]n2)cc1] | LC-MS (ESI): m/z 507.2 [M + H]+. 1H NMR (400 MHz, DMSO-d₆) δ: 8.78 (d, J = 1.2 Hz, 1H), 8.47 (s, 1H), 8.25 (bs, 1H), 8.16 (s, 1H), 8.09 (d, J = 8.8 Hz, 2H), 7.98 (s, 1H), 7.62-7.59 (m, 2H), 7.44 (d, J = 8.0 Hz, 2H), 7.39 (dd, J = 9.6 Hz, 2.0 Hz, 1H), 4.88 (s, 2H), 3.41-3.38 (m, 2H). |
| | 1-(4-(1H-1,2,4-triazol-3-yl)phenyl)- | |
| | 3-(imidazo[1,2-a]pyridin-6-yl)-7- | |
| | ((2,2,2-trifluoroethyl)amino)-3,4- | |
| | dihydropyrimido[4,5-d]pyrimidin- | |
| | 2(1H)-one | |
| Example 223 | 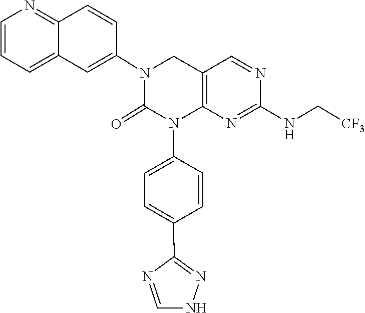 [SMILES: O=C1N(c2ccc3ncccc3c2)Cc2cnc(NCC(F)(F)F)nc2N1c1ccc(-c2nc[nH]n2)cc1] | LC-MS (ESI): m/z 518.0 [M + H]+. 1H NMR (400 MHz, DMSO-d₆) δ: 8.90 (dd, J = 4.4 Hz, 1.6 Hz, 1H), 8.47 (s, 1H), 8.39 (d, J = 8.4 Hz, 1H), 8.20 (s, 1H), 8.11 (d, J = 8.4 Hz, 2H), 8.04-8.01 (m, 2H), 7.90 (dd, J = 9.2 Hz, 2.0 Hz, 1H), 7.58-7.55 (m, 1H), 7.50 (bs, 1H), 7.49 (d, J = 8.0 Hz, 2H), 5.01 (s, 2H), 4.07-4.82 (m, 2H). |
| | 1-(4-(1H-1,2,4-triazol-3-yl)phenyl)- | |
| | 3-(quinolin-6-yl)-7-((2,2,2- | |
| | trifluoroethyl)amino)-3,4- | |
| | dihydropyrimido[4,5-d]pyrimidin- | |
| | 2(1H)-one | |
| Example 224 | 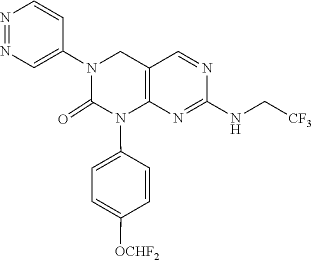 [SMILES: O=C1N(c2ccnnc2)Cc2cnc(NCC(F)(F)F)nc2N1c1ccc(OC(F)F)cc1] | LC-MS (ESI): m/z 468.1 [M + H]+. 1H NMR (400 MHz, DMSO-d₆) δ: 9.43 (s, 1H), 9.20 (d, J = 5.6 Hz, 1H), 8.20 (s, 1H), 7.69-7.67 (m, 1H), 7.55 (bs, 1H), 7.45 (d, J = 8.4 Hz, 2H), 7.33 (t, J_{HF} = 74.0 Hz, 1H), 7.29 (d, J = 8.4 Hz, 2H), 4.97 (s, 2H), 4.10-3.58 (m, 2H). |
| | 1-(4-(difluoromethoxy)phenyl)-3- | |
| | (pyridazin-4-yl)-7-((2,2,2- | |
| | trifluoroethyl)amino)-3,4- | |
| | dihydropyrimido[4,5-d]pyrimidin- | |
| | 2(1H)-one | |
| Example 225 | 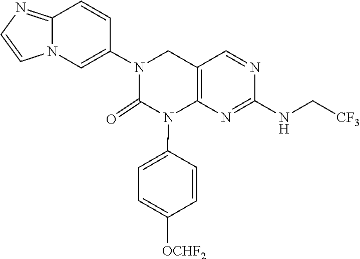 [SMILES: O=C1N(c2ccc3nccn3c2)Cc2cnc(NCC(F)(F)F)nc2N1c1ccc(OC(F)F)cc1] | LC-MS (ESI): m/z 506.0 [M + H]+. 1H NMR (400 MHz, DMSO-d₆, 0.6 HCO2H salt) δ: 8.81 (s, 1H), 8.15 (s, 1H), 8.14 (HCO2H, s, 0.6H), 8.03 (s, 1H), 7.70 (s, 1H), 7.65 (d, J = 9.6 Hz, 1H), 7.53 (bs, 1H), 7.49-7.46 (m, 1H), 7.39 (d, J = 8.4 Hz, 2H), 7.30 (t, J_{HF} = 74.0 Hz, 1H), 7.26 (d, J = 8.8 Hz, 2H), 4.86 (s, 2H), 4.07-3.28 (m, 2H). |
| | 1-(4-(difluoromethoxy)phenyl)-3- | |
| | (imidazo[1,2-a]pyridin-6-yl)-7- | |
| | ((2,2,2-trifluoroethyl)amino)-3,4- | |
| | dihydropyrimido[4,5-d]pyrimidin- | |
| | 2(1H)-one | |
| Example 226 | 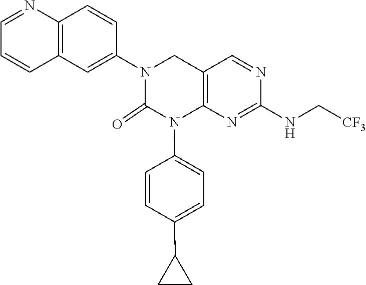 [SMILES: O=C1N(c2ccc3ncccc3c2)Cc2cnc(NCC(F)(F)F)nc2N1c1ccc(C2CC2)cc1] | LC-MS (ESI): m/z 491.2 [M + H]+. 1H NMR (400 MHz, DMSO-d₆) δ: 8.89 (dd, J = 4.4 Hz, 1.6 Hz, 1H), 8.38 (d, J = 8.8 Hz, 1H), 8.17 (s, 1H), 8.02-7.99 (m, 2H), 7.86 (dd, J = 9.2 Hz, 2.4 Hz, 1H), 7.56 (dd, J = 8.4 Hz, 4.0 Hz, 1H), 7.21 (d, J = 8.4 Hz, 2H), 7.15 (d, J = 8.4 Hz, 2H), 4.98 (s, 2H), 3.98-3.70 (m, 2H), 2.05-1.89 (m, 1H), 1.02-0.94 (m, 2H), 0.72- 0.68 (m, 2H). |
| | 1-(4-cyclopropylphenyl)-3-(quinolin- | |
| | 6-yl)-7-((2,2,2-trifluoroethyl)amino)- | |
| | 3,4-dihydropyrimido[4,5- | |
| | d]pyrimidin-2(1H)-one | |
| Example 227 | 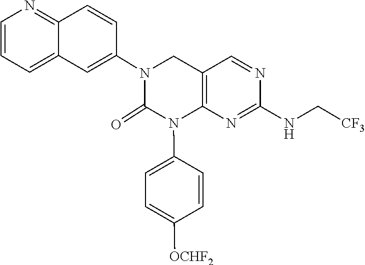 [SMILES: O=C1N(c2ccc3ncccc3c2)Cc2cnc(NCC(F)(F)F)nc2N1c1ccc(OC(F)F)cc1] | LC-MS (ESI): m/z 517.1 [M + H]+. 1H NMR (400 MHz, DMSO-d₆) δ: 8.90 (dd, J = 4.0 Hz, 1.6 Hz, 1H), 8.38 (d, J = 8.0 Hz, 1H), 8.19 (s, 1H), 8.02 (d, J = 8.4 Hz, 1H), 8.01 (s, 1H), 7.88 (dd, J = 9.2 Hz, 2.0 Hz, 1H), 7.57 (dd, J = 8.0 Hz, 4.0 Hz, 1H), 7.50 (bs, 1H), 7.44 (d, J = 8.8 Hz, 2H), 7.32 (t, J_{HF} = 73.6 Hz, 1H), 7.27 (d, J = 8.8 Hz, 2H), 5.00 (s, 2H), 4.00-3.50 (m, 2H). |
| | 1-(4-(difluoromethoxy)phenyl)-3- | |
| | (quinolin-6-yl)-7-((2,2,2- | |
| | trifluoroethyl)amino)-3,4- | |
| | dihydropyrimido[4,5-d]pyrimidin- | |
| | 2(1H)-one | |
| Example 228 | 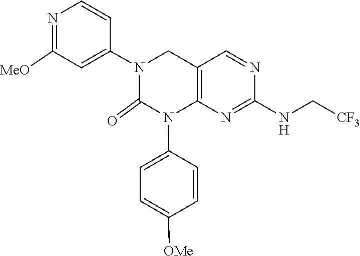 [SMILES: COc1ccc(N2C(=O)N(c3ccnc(OC)c3)Cc3cnc(NCC(F)(F)F)nc32)cc1] | LC-MS (ESI): m/z 461.2 [M + H]+. 1H NMR (400 MHz, CDCl3-d) δ: 8.04 (d, J = 6.0 Hz, 1H), 7.98 (s, 1H), 7.13 (d, J = 9.2 Hz, 2H), 6.97 (dd, J = 5.6 Hz, 2.0 Hz, 1H), 6.93 (d, J = 8.8 Hz, 2H), 6.64 (d, J = 2.0 Hz, 1H), 5.14 (bs, 1H), 4.73 (s, 2H), 3.92-3.78 (m, 2H), 3.88 (s, 3H), 3.79 (s, 3H). |
| | 1-(4-methoxyphenyl)-3-(2- | |
| | methoxypyridin-4-yl)-7-((2,2,2- | |
| | trifluoroethyl)amino)-3,4- | |
| | dihydropyrimido[4,5-d]pyrimidin- | |
| | 2(1H)-one | |
| Example 229 | 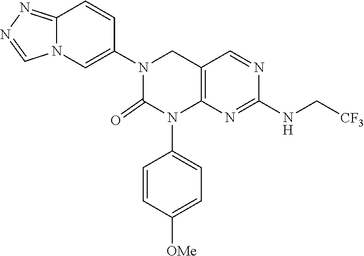 [SMILES: COc1ccc(N2C(=O)N(c3ccc4nncn4c3)Cc3cnc(NCC(F)(F)F)nc32)cc1] | LC-MS (ESI): m/z 471 [M + H]+. 1H NMR (400 MHz, CDCl3) δ: 8.80 (s, 1H), 8.27 (d, J = 0.8 Hz, 1H), 8.06 (s, 1H), 7.81 (d, J = 10.0 Hz, 1H), 7.38 (dd, J = 10.0 Hz, 2.0 Hz, 1H), 7.19 (d, J = 8.8 Hz, 2H), 7.00 (d, J = 8.8 Hz, 2H), 5.27 (bs, 1H), 4.83 (s, 2H), 4.13- 3.66 (m, 2H), 3.86 (s, 3H). |
| | 3-([1,2,4]triazolo[4,3-a]pyridin-6- | |
| | yl)-1-(4-methoxyphenyl)-7-((2,2,2- | |
| | trifluoroethyl)amino)-3,4- | |
| | dihydropyrimido[4,5-d]pyrimidin- | |
| | 2(1H)-one | |
| Example 230 | 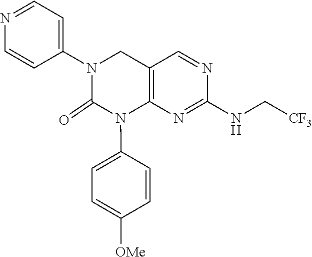 [SMILES: COc1ccc(N2C(=O)N(c3ccncc3)Cc3cnc(NCC(F)(F)F)nc32)cc1] | LC-MS (ESI): m/z 431.0 [M + H]+. 1H NMR (400 MHz, CDCl3) δ: 8.61 (s, 2H), 8.09 (s, 1H), 7.47 (s, 2H), 7.20 (d, J = 8.8 Hz, 2H), 7.01 (d, J = 8.8 Hz, 2H), 5.35 (bs, 1H), 4.87 (s, 2H), 4.18-3.67 (m, 2H), 3.86 (s, 3H). |
| | 1-(4-methoxyphenyl)-3-(pyridin-4- | |
| | yl)-7-((2,2,2-trifluoroethyl)amino)- | |
| | 3,4-dihydropyrimido[4,5- | |
| | d]pyrimidin-2(1H)-one | |
| Example 231 | 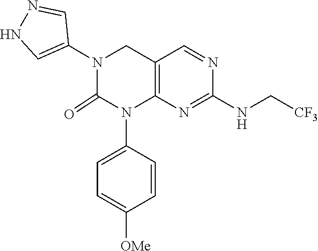 [SMILES: COc1ccc(N2C(=O)N(c3cn[nH]c3)Cc3cnc(NCC(F)(F)F)nc32)cc1] | LC-MS (ESI): m/z 420.1 [M + H]+. 1H NMR (400 MHz, DMSO-d₆) δ: 12.75 (bs, 1H), 8.13 (s, 1H), 7.82 (s, 2H), 7.44 (s, 1H), 7.18 (d, J = 8.8 Hz, 2H), 6.99 (d, J = 8.9 Hz, 2H), 4.81 (s, 2H), 4.14-3.66 (m, 2H), 3.80 (s, 3H). |
| | 1-(4-methoxyphenyl)-3-(1H-pyrazol- | |
| | 4-yl)-7-((2,2,2-trifluoroethyl)amino)- | |
| | 3,4-dihydropyrimido[4,5- | |
| | d]pyrimidin-2(1H)-one | |
| | (synthesized from 4-iodo-1-((2- | |
| | (trimethylsilyl)ethoxy)methyl)-1H- | |
| | pyrazole, and de-protection with TFA | |
| | via general procedure V (Step F)) | |
| Example 232 | 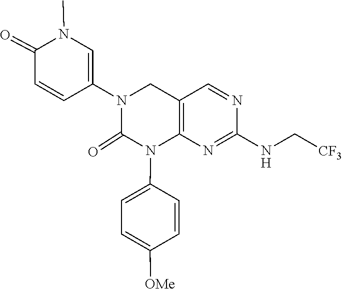 [SMILES: COc1ccc(N2C(=O)N(c3ccc(=O)n(C)c3)Cc3cnc(NCC(F)(F)F)nc32)cc1] | LC-MS (ESI): m/z 461.0 [M + H]+. 1H NMR (400 MHz, CDCl3) δ: 8.01 (s, 1H), 7.44 (d, J = 2.8 Hz, 1H), 7.39 (dd, J = 9.6 Hz, 3.2 Hz, 1H), 7.15 (d, J = 8.8 Hz, 2H), 6.98 (d, J = 8.8 Hz, 2H), 6.61 (d, J = 9.6 Hz, 1H), 5.26 (bs, 1H), 4.68 (s, 2H), 4.03-3.68 (m, 2H), 3.85 (s, 3H), 3.54 (s, 3H). |
| | 1-(4-methoxyphenyl)-3-(1-methyl-6- | |
| | oxo-1,6-dihydropyridin-3-yl)-7- | |
| | ((2,2,2-trifluoroethyl)amino)-3,4- | |
| | dihydropyrimido[4,5-d]pyrimidin- | |
| | 2(1H)-one | |
| Example 233 | 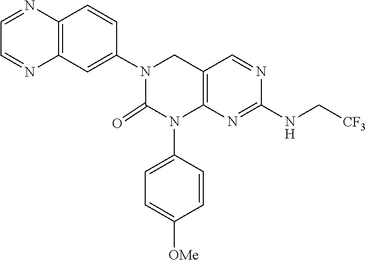 [SMILES: COc1ccc(N2C(=O)N(c3ccc4nccnc4c3)Cc3cnc(NCC(F)(F)F)nc32)cc1] | LC-MS (ESI): m/z 482.0 [M + H]+. 1H NMR (400 MHz, DMSO-d₆) δ: 8.96 (d, J = 1.6 Hz, 1H), 8.93 (d, J = 1.6 Hz, 1H), 8.18 (s, 1H), 8.08 (d, J = 9.2 Hz, 1H), 8.07 (s, 1H), 8.01 (dd, J = 9.2 Hz, 2.4 Hz, 1H), 7.55 (bs, 1H), 7.29 (d, J = 8.8 Hz, 2H), 7.01 (d, J = 8.8 Hz, 2H), 5.05 (s, 2H), 4.13-3.65 (m, 2H), 3.80 (s, 3H). |
| | 1-(4-methoxyphenyl)-3-(quinoxalin- | |
| | 6-yl)-7-((2,2,2-trifluoroethyl)amino)- | |
| | 3,4-dihydropyrimido[4,5- | |
| | d]pyrimidin-2(1H)-one | |
| Example 234 | 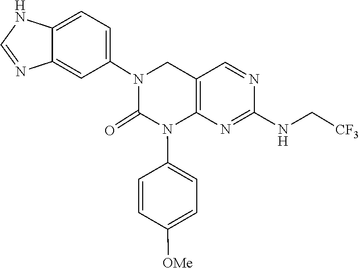 [SMILES: COc1ccc(N2C(=O)N(c3ccc4[nH]cnc4c3)Cc3cnc(NCC(F)(F)F)nc32)cc1] | LC-MS (ESI): m/z 470.1 [M + H]+. 1H NMR (400 MHz, DMSO-d₆) δ: 8.35-8.21 (m, 2H), 8.11 (s, 1H), 7.72-7.58 (m, 2H), 7.53-7.37 (m, 1H), 7.32-7.17 (m, 1H), 7.22 (d, J = 8.0 Hz, 2H), 6.98 (d, J = 8.0 Hz, 2H), 4.84 (s, 2H), 3.79 (s, 3H), 4.00-3.50 (m, 2H). |
| | 3-(1H-benzo[d]imidazol-5-yl)-1-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| | (synthesized from 5-bromo-1-((2- | |
| | (trimethylsilyl)ethoxy)methyl)-1H- | |
| | benzo[d]imidazole, and de-protection | |
| | with TFA via general procedure V | |
| | (Step F)) | |
| Example 421 | 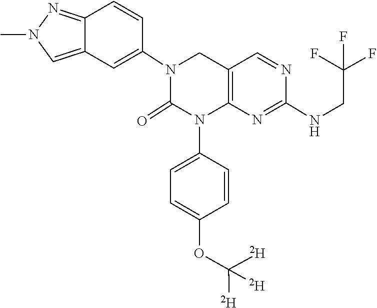 [SMILES: [2H]C([2H])([2H])Oc1ccc(N2C(=O)N(c3ccc4nn(C)cc4c3)Cc3cnc(NCC(F)(F)F)nc32)cc1] | LC-MS: m/z 487 (M + H)+. 1H NMR (400 MHz, DMSO-d₆) δ: 8.35 (s, 1H), 8.11 (s, 1H), 7.69 (d, J = 1.4 Hz, 1H), 7.58 (d, J = 9.2 Hz, 1H), 7.48 (s, 1H), 7.26 (dd, J = 9.2 Hz, 2.0 Hz, 1H), 7.21 (d, J = 8.8 Hz, 2H), 7.02-6.93 (m, 2H), 4.83 (s, 2H), 4.17 (s, 3H), 3.92 (s, 2H). |
| | 1-(4-(methoxy-d3)phenyl)-3-(2- | |
| | methyl-2H-indazol-5-yl)-7-((2,2,2- | |
| | trifluoroethyl)amino)-3,4- | |
| | dihydropyrimido[4,5-d]pyrimidin- | |
| | 2(1H)-one | |
| Example 422 | 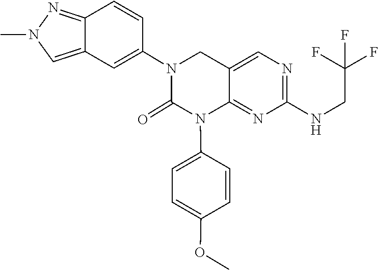 [SMILES: COc1ccc(N2C(=O)N(c3ccc4nn(C)cc4c3)Cc3cnc(NCC(F)(F)F)nc32)cc1] | LC-MS: m/z 484 (M + H)+. 1H NMR (400 MHz, DMSO-d₆) δ: 8.35 (s, 1H), 8.11 (s, 1H), 7.69 (s, 1H), 7.57 (d, J = 9.1 Hz, 1H), 7.44 (s, 1H), 7.26 (dd, J = 9.1 Hz, 1.8 Hz, 1H), 7.21 (d, J = 8.7 Hz, 2H), 6.98(d, J = 8.8 Hz, 2H), 4.83 (s, 2H), 4.17 (s, 3H), 3.95 (s, 2H), 3.78 (s, 3H). |
| | 1-(4-methoxyphenyl)-3-(2-methyl- | |
| | 2H-indazol-5-yl)-7-((2,2,2- | |
| | trifluoroethyl)amino)-3,4- | |
| | dihydropyrimido[4,5-d]pyrimidin | |
| | 2(1H)-one | |
| Example 423 | 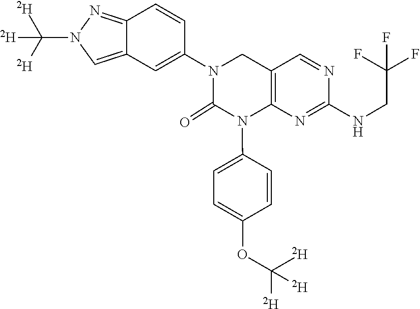 [SMILES: [2H]C([2H])([2H])Oc1ccc(N2C(=O)N(c3ccc4nn(C([2H])([2H])[2H])cc4c3)Cc3cnc(NCC(F)(F)F)nc32)cc1] | LC-MS: m/z 490 (M + H)+. 1H NMR (400 MHz, DMSO-d₆) δ: 8.35 (s, 1H), 8.11 (s, 1H), 7.69 (d, J = 1.4 Hz, 1H), 7.58 (d, J = 9.2 Hz, 1H), 7.45 (s, 1H), 7.26 (dd, J = 9.2 Hz, 2.0 Hz, 1H), 7.21 (d, J = 8.8 Hz, 2H), 6.97 (d, J = 8.9 Hz, 2H), 4.83 (s, 2H), 3.89 (s, 2H). |
| | 1-(4-(methoxy-d3)phenyl)-3-(2- | |
| | (methyl-d3)-2H-indazol-5-yl)-7- | |
| | ((2,2,2-trifluoroethyl)amino)-3,4- | |
| | dihydropyrimido[4,5-d]pyrimidin | |
| | 2(1H)-one | |
| Example 424 | 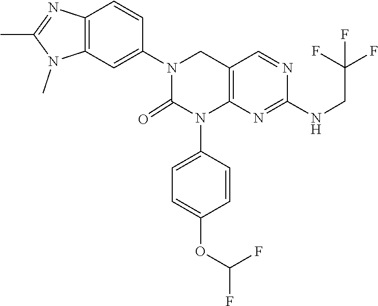 [SMILES: Cc1nc2ccc(N3Cc4cnc(NCC(F)(F)F)nc4N(c4ccc(OC(F)F)cc4)C3=O)cc2n1C] | LC-MS: m/z 534 (M + H)+. 1H NMR (400 MHz, DMSO-d₆) δ: 8.14 (s, 1H), 7.56 (d, J = 1.7 Hz, 1H), 7.51 (d, J = 8.5 Hz, 1H), 7.29 (t, JRF = 76 Hz, 1H), 7.38- 7.11 (m, 5H), 4.84 (s, 2H), 3.73 (s, 2H), 3.71(s, 3H), 2.52 (s, 3H). |
| | 1-(4-(difluoromethoxy)phenyl)-3- | |
| | (1,2-dimethyl-1H-benzo[dlimidazol- | |
| | 6-yl)-7-((2,2,2-trifluoroethyl)amino)- | |
| | 3,4-dihydropyrimido[4,5- | |
| | d]pyrimidin-2(1H)-one | |

### General Procedure VI

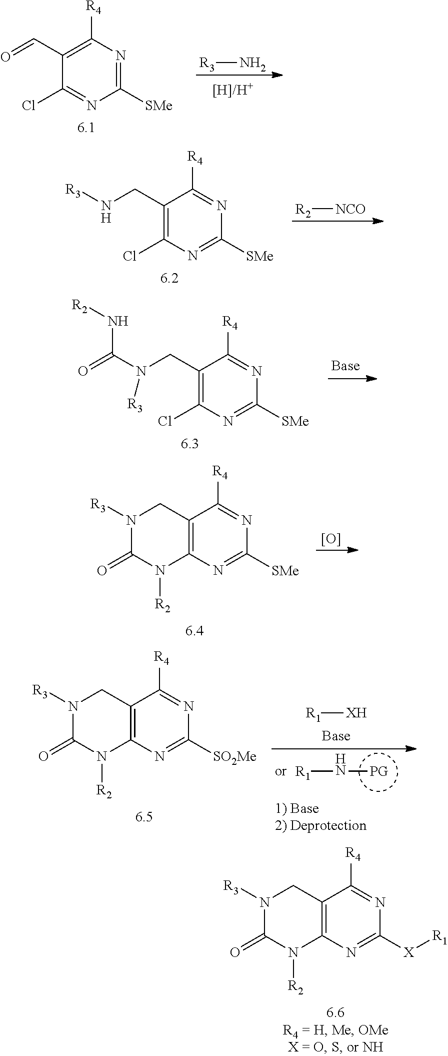

Compounds of structure 6.6 were obtained through the scheme depicted as General Procedure VI. Beginning with aldehyde 6.1, the desired R₃ group was introduced using a reductive amination to generate compound 6.2. The desired R₂ group was introduced by reacting amine 6.2 with the appropriate isocyanate to generate acyclic urea 6.3. Acyclic urea 6.3 was converted to cyclic urea 6.4 using a base mediated aromatic substitution reaction. Aryl thiol 6.4 was then oxidized to sulfone 6.5 and the desired R₁ group (optionally protected when X═NH) was introduced using a base mediated aromatic substitution reaction to afford compound 6.6.

### Preparation of Example 235 Via General Procedure VI

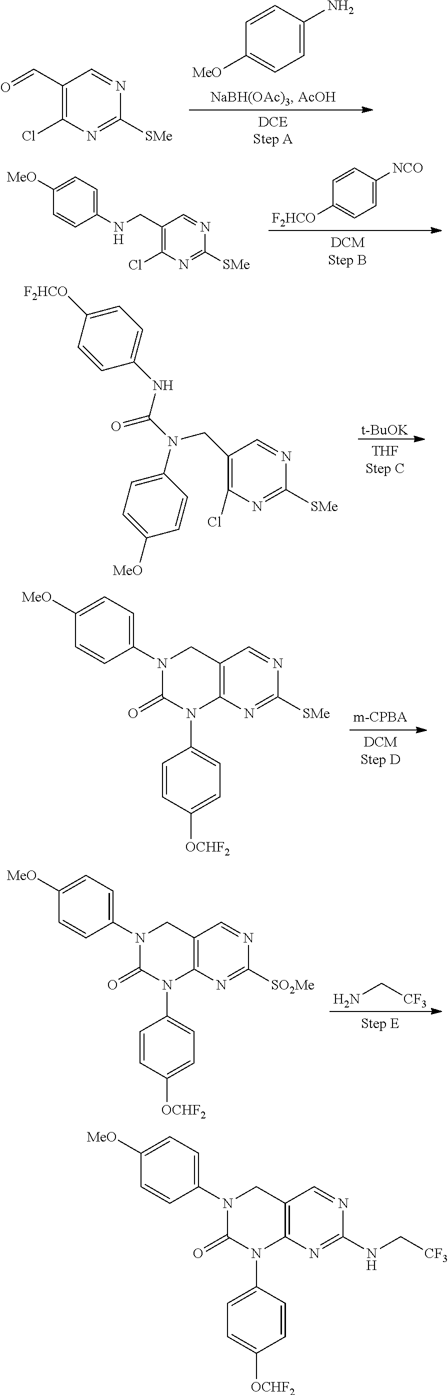

### Step A: N-((4-chloro-2-(methylthio)pyrimidin-5-yl)methyl)-4-methoxy aniline

4-Chloro-2-(methylthio)pyrimidine-5-carbaldehyde (0.94 g, 5.0 mmol, 1.0 eq.), 4-methoxyaniline (0.62 g, 5.0 mmol, 1.0 eq.) and AcOH (0.9 mL, 15.0 mmol, 3.0 eq.) was dissolved in DCE (6 mL), the resulting mixture stirred at room temperature for 3 hrs, then NaBH(OAc)₃ (1.17 g, 5.5 mmol, 1.1 eq.) was added in several portions at 0° C., after addition, the reaction mixture was allowed to warm to room temperature and stirred overnight. The reaction mixture was quenched with ice water (10 mL) and extracted with DCM (20 mL×3), the combined organic layers were washed with brine (30 mL) and dried over Na₂SO₄, concentrated under reduced pressure, the residue was purified by flash column chromatography on silica gel to give N-((4-chloro-2-(methylthio)pyrimidin-5-yl)methyl)-4-methoxyaniline (1.33 g, 90% yield) as a gray solid. LC-MS (ESI): m/z 296 [M+H]⁺.

### Step B: 1-((4-chloro-2-(methylthio)pyrimidin-5-yl)methyl)-3-(4-(difluoromethoxy)phenyl)-1-(4-methoxyphenyl)urea

A solution of N-((4-chloro-2-(methylthio)pyrimidin-5-yl)methyl)-4-methoxyaniline (130 mg, 0.44 mmol, 1.0 eq.) in DCM (5 mL) was added 1-(difluoromethoxy)-4-isocyanatobenzene (163 mg, 0.88 mmol, 2.0 eq.) and stirred at room temperature overnight. Then the reaction mixture was concentrated under reduced pressure, the residue was purified by flash column chromatography on silica gel to afford 1-((4-chloro-2-(methylthio)pyrimidin-5-yl)methyl)-3-(4-(difluoromethoxy)phenyl)-1-(4-methoxyphenyl)urea (120 mg, 57% yield) as a yellow solid. LC-MS (ESI): m/z 481 [M+H]⁺.

### Step C: 1-(4-(difluoromethoxy)phenyl)-3-(4-methoxyphenyl)-7-(methylthio)-3,4-dihydropyrimido[4,5-d]pyrimidin-2(1H)-one

A solution of 1-((4-chloro-2-(methylthio)pyrimidin-5-yl)methyl)-3-(4-(difluoromethoxy)phenyl)-1-(4-methoxyphenyl)urea (120 mg, 0.25 mmol, 1.0 eq.) in THF (5 mL) was added t-BuOK (84 mg, 0.75 mmol, 3.0 eq.) and stirred for 1 hr at room temperature. Then the reaction mixture was treated with ice water (10 mL), extracted with EtOAc (10 mL×3), the combined organic layers were dried over Na₂SO₄, concentrated under reduced pressure, the residue was purified by flash column chromatography in silica gel to afford 1-(4-(difluoromethoxy)phenyl)-3-(4-methoxyphenyl)-7-(methylthio)-3,4-dihydropyrimido[4,5-d]pyrimidin-2(1H)-one (80 mg, 72% yield) as yellow oil. LC-MS (ESI): m/z 445 [M+H]⁺.

1-(4-(Difluoromethoxy)phenyl)-3-(4-methoxyphenyl)-7-(2,2,2-trifluoroethylamino)-3,4-dihydropyrimido[4,5-d]pyrimidin-2(1H)-one (Example 235) was synthesized from 1-(4-(difluoromethoxy)phenyl)-3-(4-methoxyphenyl)-7-(methylthio)-3,4-dihydropyrimido[4,5-d]pyrimidin-2(1H)-one and 2,2,2-trifluoroethanamine via general procedure IV (Step D, E).

¹H NMR (400 MHz, DMSO-d₆) δ (ppm): 8.12 (s, 1H), 7.69-7.39 (m, 1H), 7.38-7.31 (m, 4H), 7.30 (t, J_{HF}=74.0 Hz, 1H), 7.24 (d, J=8.8 Hz, 2H), 6.96 (d, J=8.8 Hz, 2H), 4.76 (s, 2H), 4.19-3.49 (m, 2H), 3.77 (s, 3H).

LC-MS (ESI): m/z 496 [M+H]⁺.

### Preparation of Example 236 Via General Procedure VI with Final Deprotection

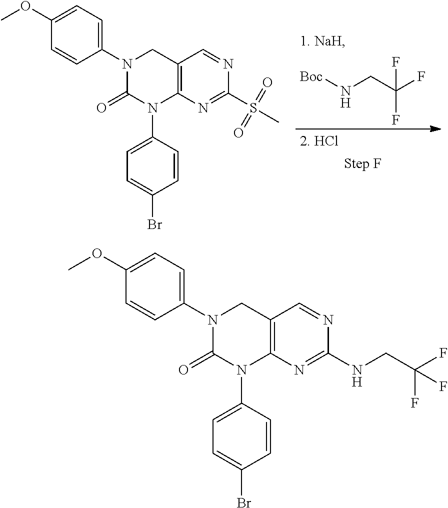

### 1-(4-bromophenyl)-3-(4-methoxyphenyl)-7-((2,2,2-trifluoroethyl)amino)-3,4-dihydropyrimido[4,5-d]pyrimidin-2(1H)-one

To a solution of tert-butyl (2,2,2-trifluoroethyl)carbamate (150 mg, 0.753 mmol) in THF (2.510 ml) was added sodium hydride (36.1 mg, 0.904 mmol) at 0° C. and stirred for 10 minutes then warm up to rt for another 30 minutes. 0.5 ml of this mixture (about 0.15 mmol, 1.5 eq.) was added dropwise into another reaction vial containing a solution of 1-(4-bromophenyl)-3-(4-methoxyphenyl)-7-(methylsulfonyl)-3,4-dihydropyrimido[4,5-d]pyrimidin-2(1H)-one (50 mg, 0.102 mmol, 1.0 equiv, generated using general procedure VI steps A-D) in THF (0.5 ml) and the resulting reaction mixture was stirred for 30 minutes until reaction completed monitored by LCMS to form tert-butyl (8-(4-bromophenyl)-6-(4-methoxyphenyl)-7-oxo-5,6,7,8-tetrahydropyrimido[4,5-d]pyrimidin-2-yl)(2,2,2-trifluoroethyl)carbamate. To this reaction mixture added HCl (150 μl, 0.600 mmol) in dioxane then the resulting mixture was heated to 60° C. for another 30 min until Boc deprotection was complete. Then the reaction was quenched with solid sodium bicarbonate (86 mg, 1.022 mmol) and mixed with silica gel. Organic solvent was removed after concentration. The crude material was dry-loaded and purified by column chromatography to afford 1-(4-bromophenyl)-3-(4-methoxyphenyl)-7-((2,2,2-trifluoroethyl)amino)-3,4-dihydropyrimido[4,5-d]pyrimidin-2(1H)-one. (Example 236)

1H NMR (400 MHz, Chloroform-d) δ 8.00 (s, 1H), 7.57 (d, J=8.7 Hz, 2H), 7.28 (d, J=9.0 Hz, 2H), 7.17 (d, J=8.7 Hz, 2H), 6.93 (d, J=9.0 Hz, 2H), 5.14 (brs, 1H), 4.75 (s, 2H), 3.90 (brs, 2H), 3.81 (s, 3H).

LC-MS (ESI): m/z 510.0 [M+H]⁺.

The procedure set forth above for General Procedure VI was used to synthesize the following compounds by using appropriate starting materials:

| Cpd No. | Structure | Characterization |
|---|---|---|
| Example 237 | 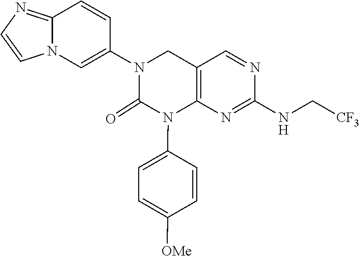 [SMILES: COc1ccc(N2C(=O)N(c3ccc4nccn4c3)Cc3cnc(NCC(F)(F)F)nc32)cc1] | LC-MS (ESI): m/z 470.2 [M + H]+. 1H NMR (400 MHz, DMSO) δ: 8.75 (d, J = 1.2 Hz, 1H) 8.35 (s, 1H) 8.12 (s, 1H), 7.96 (s, 1H), 7.61 (d, J = 1.0 Hz, 1H), 7.58 (d, J = 9.2 Hz, 1H), 7.36 (dd, J = 9.6 Hz, 2.0 Hz, 1H), 7.21 (d, J = 8.8 Hz, 2H), 6.99 (d, J = 9.2 Hz, 2H), 4.84 (s, 2H), 4.15-3.86 (m, 2H), 3.79 (s, 3H). |
| | 3-(imidazo[1,2-a]pyridin-6-yl)-1- | |
| | (4-methoxyphenyl)-7-((2,2,2- | |
| | trifluoroethyl)amino)-3,4,4a,8a- | |
| | tetrabydropyrimido[4,5- | |
| | d]pyrimidin-2(1H)-one | |
| Example 238 | 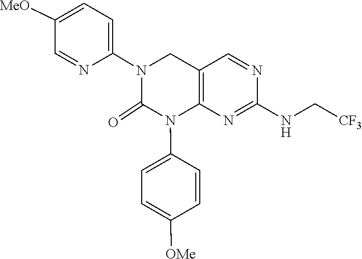 [SMILES: COc1ccc(N2C(=O)N(c3ccc(OC)cn3)Cc3cnc(NCC(F)(F)F)nc32)cc1] | LC-MS (ESI): m/z 461.0 [M + H]+. 1H NMR (400 MHz, CDCl3) δ: 8.10 (d, J = 3.2 Hz, 1H), 8.07 (s, 1H), 7.72 (d, J = 8.8 Hz, 1H), 7.23 (dd, J = 9.2 Hz, 3.2 Hz, 1H), 7.21 (d, J = 8.8 Hz, 2H), 7.00 (d, J = 8.8 Hz, 2H), 5.22- 5.11 (m, 1H), 5.02 (s, 2H), 4.01-3.91 (m, 2H), 3.86 (s, 3H), 3.85 (s, 3H). |
| | 1-(4-methoxyphenyl)-3-(5- | |
| | methoxypyridin-2-yl)-7-((2,2,2- | |
| | trifluoroethyl)amino)-3,4- | |
| | dihydropyrimido[4,5-d]pyrimidin- | |
| | 2(1H)-one | |
| Example 239 | 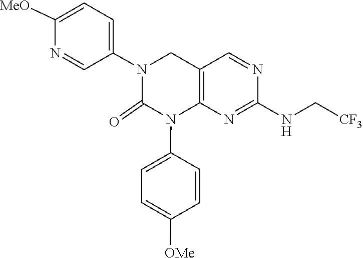 [SMILES: COc1ccc(N2C(=O)N(c3ccc(OC)nc3)Cc3cnc(NCC(F)(F)F)nc32)cc1] | LC-MS (ESI): m/z 461.2 [M + H]+. 1H NMR (400 MHz, DMSO-d₆) δ: 8.23 (d, J = 2.4 Hz, 1H), 8.10 (s, 1H), 7.79 (dd, J = 8.8 Hz, 2.8 Hz, 1H), 7.45 (s, 1H), 7.20 (d, J = 8.8 Hz, 2H), 6.98 (d, J = 8.8 Hz, 2H), 6.87 (d, J = 9.2 Hz, 1H), 4.79 (s, 2H), 4.05-3.62 (m, 2H), 3.86 (s, 3H), 3.78 (s, 3H). |
| | 1-(4-methoxyphenyl)-3-(6- | |
| | methoxypyridin-3-yl)-7-((2,2,2- | |
| | trifluoroethyl)amino)-3,4- | |
| | dihydropyrimido[4,5-d]pyrimidin- | |
| | 2(1H)-one | |
| Example 240 | 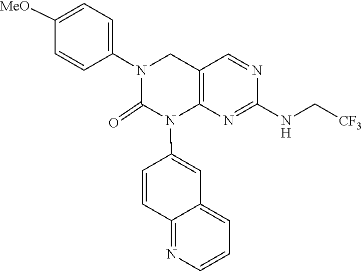 [SMILES: COc1ccc(N2Cc3cnc(NCC(F)(F)F)nc3N(c3ccc4ncccc4c3)C2=O)cc1] | LC-MS (ESI): m/z 481.2 [M + H]+. 1H NMR (400 MHz, CDCl3) δ: 8.96 (dd, J = 4.4 Hz, 2.0 Hz, 1H), 8.18- 8.15 (m, 2H), 8.04 (s, 1H), 7.83 (d, J = 2.0 Hz, 1H), 7.63 (dd, J = 8.8 Hz, 2.4 Hz, 1H), 7.43 (dd, J = 8.4 Hz, 4.4 Hz, 1H), 7.32 (d, J = 8.8 Hz, 2H), 6.94 (d, J = 9.2 Hz, 2H), 5.13 (bs, 1H), 4.82 (s, 2H), 4.00- 3.63 (m, 2H), 3.82 (s, 3H). |
| | 3-(4-methoxyphenyl)-1-(quinolin- | |
| | trifluoroethyl)amino)-3,4- | |
| | dihydropyrimido[4,5-d]pyrimidin- | |
| | 2(1H)-one | |
| Example 241 | 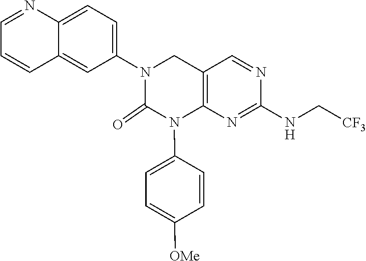 [SMILES: COc1ccc(N2C(=O)N(c3ccc4ncccc4c3)Cc3cnc(NCC(F)(F)F)nc32)cc1] | LC-MS (ESI): m/z 481.0 [M + H]+. 1H NMR (400 MHz, DMSO-d₆) δ: 8.89 (s, 1H), 8.37 (d, J = 8.0 Hz, 1H), 8.16 (s, 1H), 8.01 (d, J = 9.6 Hz, 2H), 7.86 (d, J = 8.8 Hz, 1H), 7.56 (dd, J = 8.0 Hz, 4.0 Hz, 1H), 7.50 (bs, 1H), 7.26 (d, J = 8.0 Hz, 2H), 7.00 (d, J = 8.4 Hz, 2H), 4.98 (s, 2H), 4.15-3.85 (m, 2H), 3.80 (s, 3H). |
| | 1-(4-methoxyphenyl)-3-(quinolin- | |
| | trifluoroethyl)amino)-3,4- | |
| | dihydropyrimido[4,5-d]pyrimidin- | |
| | 2(1H)-one | |
| Example 242 | 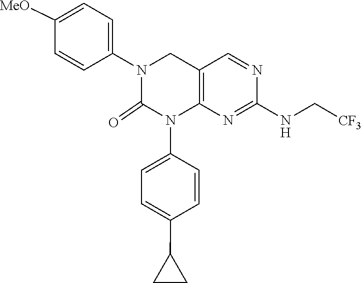 [SMILES: COc1ccc(N2Cc3cnc(NCC(F)(F)F)nc3N(c3ccc(C4CC4)cc3)C2=O)cc1] | LC-MS (ESI): m/z 470.0 [M + H]+. 1H NMR (400 MHz, DMSO-d₆, 0.7 HCOOH salt.) δ: 8.41 (s, 0.7H), 8.10 (s, 1H), 7.45 (bs, 1H), 7.33 (d, J = 8.8 Hz, 2H), 7.13 (s, 4H), 6.96 (d, J = 8.8 Hz, 2H), 4.75 (s, 2H), 4.19- 3.63 (m, 2H), 3.77 (s, 3H), 2.01-1.91 (m, 1H), 1.03-0.93 (m, 2H), 0.74- 0.64 (m, 2H). |
| | 1-(4-cyclopropylphenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 243 | 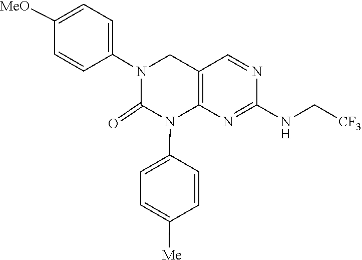 [SMILES: COc1ccc(N2Cc3cnc(NCC(F)(F)F)nc3N(c3ccc(C)cc3)C2=O)cc1] | LC-MS (ESI): m/z 444.0 [M + H]+. 1H NMR (400 MHz, DMSO-d₆) δ: 8.10 (s, 1H), 7.50 (bs, 1H), 7.33 (d, J = 9.2 Hz, 2H), 7.24 (d, J = 8.0 Hz, 2H), 7.15 (d, J = 8.4 Hz, 2H), 6.96 (d, J = 8.8 Hz, 2H), 4.75 (s, 2H), 4.15-3.73 (m, 2H), 3.77 (s, 3H), 2.35 (s, 3H). |
| | 3-(4-methoxyphenyl)-1-(p-tolyl)- | |
| | 7-((2,2,2-trifluoroethyl)amino)- | |
| | 3,4-dihydropyrimido[4,5- | |
| | d]pyrimidin-2(1H)-one | |
| Example 244 | 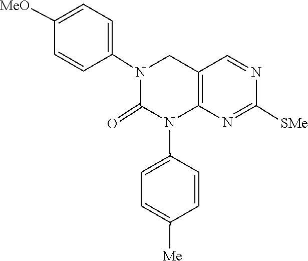 [SMILES: COc1ccc(N2Cc3cnc(SC)nc3N(c3ccc(C)cc3)C2=O)cc1] | LC-MS (ESI): m/z 393.0 [M + H]+. 1H NMR (400 MHz, DMSO-d₆) δ: 8.33 (s, 1H), 7.35 (d, J = 8.8 Hz, 2H), 7.26 (d, J = 8.0 Hz, 2H), 7.19 (d, J = 8.0 Hz, 2H), 6.98 (d, J = 8.8 Hz, 2H), 4.87 (s, 2H), 3.77 (s, 3H), 2.36 (s, 3H), 2.20 (s, 3H). |
| | 3-(4-methoxyphenyl)-7- | |
| | (methylthio)-1-(p-tolyl)-3,4- | |
| | dihydropyrimido[4,5-d]pyrimidin- | |
| | 2(1H)-one | |
| Example 245 | 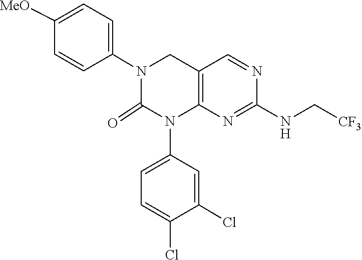 [SMILES: COc1ccc(N2Cc3cnc(NCC(F)(F)F)nc3N(c3ccc(Cl)c(Cl)c3)C2=O)cc1] | LC-MS (ESI): m/z 498.0 [M + H]+. 1H NMR (400 MHz, CDCl3) δ: 8.01 (s, 1H), 7.52 (d, J = 8.4 Hz, 1H), 7.44 (d, J = 2.4 Hz, 1H), 7.31-7.26 (m, 2H), 7.16 (dd, J = 8.4 Hz, 2.4 Hz, 1H), 6.93 (d, J = 8.8 Hz, 2H), 5.43- 5.12 (m, 1H), 4.75 (s, 2H), 4.05-3.76 (m, 2H), 3.82 (s, 3H). |
| | 1-(3,4-dichlorophenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 246 (R₄ = OMe) | 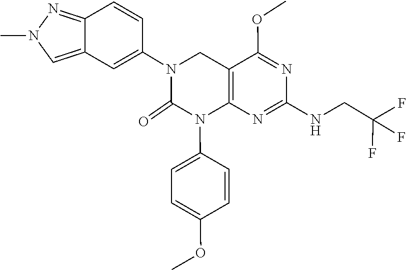 [SMILES: COc1ccc(N2C(=O)N(c3ccc4nn(C)cc4c3)Cc3c(OC)nc(NCC(F)(F)F)nc32)cc1] | LC-MS (ESI): m/z 514.2 (M + H)+. 1H NMR (400 MHz, DMSO-d₆) δ: 8.33 (s, 1H), 7.71 (d, J = 1.2 Hz, 1H), 7.57 (d, J = 9.6 Hz, 1H), 7.43 (br s, 1H), 7.25 (dd, J = 9.2 Hz, 1.6 Hz, 1H), 7.16 (d, J = 8.8 Hz, 2H), 6.95 (d, J = 8.8 Hz, 2H), 4.66 (s, 2H), 4.16 (s, 3H), 4.05-3.60 (m, 2H), 3.87 (s, 3H), 3.77 (s, 3H). |
| | 5-methoxy-1-(4-methoxyphenyl)- | |
| | 3-(2-methyl-2H-indazol-5-yl)-7- | |
| | ((2,2,2-trifluoroethyl)amino)-3,4- | |
| | dihydropyrimido[4,5-d]pyrimidin- | |
| | 2(1H)-one | |
| Example 247 (R₄ = OMe) | 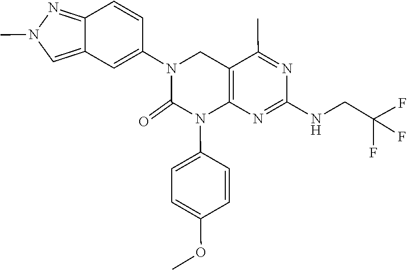 [SMILES: COc1ccc(N2C(=O)N(c3ccc4nn(C)cc4c3)Cc3c(C)nc(NCC(F)(F)F)nc32)cc1] | LC-MS (ESI): m/z 498.2 (M + H)+. 1H NMR (400 MHz, DMSO-d₆) δ: 8.35 (s, 1H), 7.73 (s, 1H), 7.58 (d, J = 9.2 Hz, 1H), 7.45 (br s, 1H), 7.27 (dd, J = 9.2 Hz, 2.0 Hz, 1H), 7.16 (d, J = 8.8 Hz, 2H), 6.96 (d, J = 8.8 Hz, 2H), 4.82 (s, 2H), 4.17 (s, 3H), 4.00- 3.60 (m, 2H), 3.78 (s, 3H), 2.21 (s, 3H). |
| | 1-(4-methoxyphenyl)-5-methyl-3- | |
| | (2-methyl-2H-indazol-5-yl)-7- | |
| | ((2,2,2-trifluoroethyl)amino)-3,4- | |
| | dihydropyrimido[4,5-d]pyrimidin- | |
| | 2(1H)-one | |
| Example 248 | 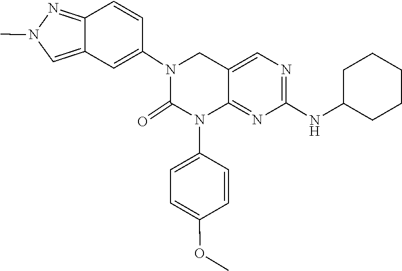 [SMILES: COc1ccc(N2C(=O)N(c3ccc4nn(C)cc4c3)Cc3cnc(NC4CCCCC4)nc32)cc1] | LC-MS (ESI): m/z 484.2 (M + H)+. 1H NMR (400 MHz, DMSO-d₆) δ: 8.34 (s, 1H), 8.01 (s, 1H), 7.68 (s, 1H), 7.57 (d, J = 8.8 Hz, 1H), 7.25 (dd, J = 9.2 Hz, 1.6 Hz, 1H), 7.19 (d, J = 8.8 Hz, 2H), 6.96 (d, J = 8.8 Hz, 2H), 6.90-6.45 (m, 1H), 4.77 (s, 2H), 4.16 (s, 3H), 3.78 (s, 3H), 3.01-2.80 (m, 1H), 1.80-1.43 (m, 5H), 1.25- 0.83 (m, SH). |
| | 7-(cyclohexylamino)-1-(4- | |
| | methoxyphenyl)-3-(2-methyl-2H- | |
| | indazol-5-yl)-3,4- | |
| | dihydropyrimido[4,5-d]pyrimidin- | |
| | 2(1H)-one | |
| Example 249 (via Step F) | 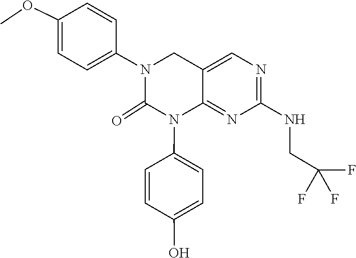 [SMILES: COc1ccc(N2Cc3cnc(NCC(F)(F)F)nc3N(c3ccc(O)cc3)C2=O)cc1] | LC-MS (ESI): m/z 446.1 [M + H]⁺. 1H NMR (400 MHz, Chloroform-d) δ 7.88 (s, 1H), 7.20 (d, J = 9.0 Hz, 2H), 7.01 (d, J = 8.8 Hz, 2H), 6.86 (d, J = 9.0 Hz, 2H), 6.82 (d, J = 8.8 Hz, 2H), 4.68 (s, 2H), 3.75 (s, J = 1.2 Hz, 3H), 3.32 − 3.30 (m, 2H). |
| | 1-(4-hydroxyphenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 250 (via Step F) | 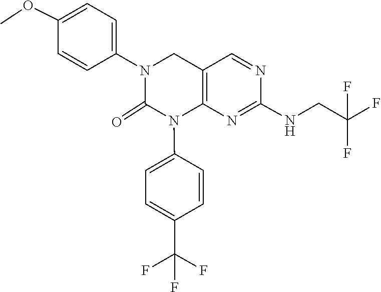 [SMILES: COc1ccc(N2Cc3cnc(NCC(F)(F)F)nc3N(c3ccc(C(F)(F)F)cc3)C2=O)cc1] | LC-MS (ESI): m/z 498.1 [M + H]⁺. 1H NMR (400 MHz, Chloroform-d) δ 7.93 (s, 1H), 7.73 (d, J = 8.3 Hz, 2H), 7.42 (d, J = 8.3 Hz, 2H), 7.28 (d, J = 9.0 Hz, 2H), 6.94 (d, J = 9.0 Hz, 2H), 4.79 (s, 2H), 3.82 (s, 3H), 3.75 (brs, 2H). |
| | 3-(4-methoxyphenyl)-7-((2,2,2- | |
| | trifluoroethyl)amino)-1-(4- | |
| | (trifluoromethyl)phenyl)-3,4- | |
| | dihydropyrimido[4,5-d]pyrimidin- | |
| | 2(1H)-one | |
| Example 251 (via Step F) | 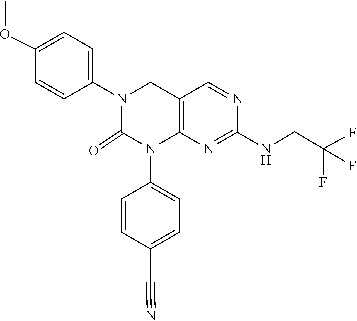 [SMILES: COc1ccc(N2Cc3cnc(NCC(F)(F)F)nc3N(c3ccc(C#N)cc3)C2=O)cc1] | LC-MS (ESI): m/z 455.1 [M + H]⁺. 1H NMR (400 MHz, Chloroform-d) δ 7.99 (s, 1H), 7.75 (d, J = 8.5 Hz, 2H), 7.43 (d, J = 8.5 Hz, 2H), 7.27 (d, J = 8.9 Hz, 2H), 6.94 (d, J = 8.9 Hz, 2H), 4.79 (s, 2H), 3.82 (s, 3H), 3.75 (brs, 2H). |
| | 4-(3-(4-methoxyphenyl)-2-oxo-7- | |
| | ((2,2,2-trifluoroethyl)amino)-3,4- | |
| | dihydropyrimido[4,5-d]pyrimidin- | |
| | 1(2H)-yl)benzonitrile | |
| Example 252 (via Step F) | 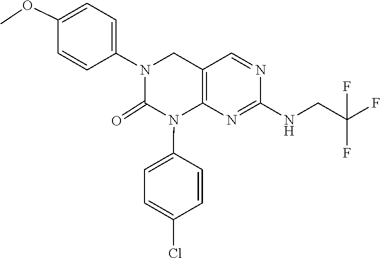 [SMILES: COc1ccc(N2Cc3cnc(NCC(F)(F)F)nc3N(c3ccc(Cl)cc3)C2=O)cc1] | LC-MS (ESI): m/z 464.0 [M + H]⁺. 1H NMR (400 MHz, Chloroform-d) δ 7.91 (s, 1H), 7.43 (d, J = 8.7 Hz, 2H), 7.27 (d, J = 9.0 Hz, 2H), 7.21 J = 8.7 Hz, 2H), 6.94 (d, J = 9.0 (d, Hz, 2H), 4.77 (s, 2H), 3.82 (s, 3H), 3.77 (brs, 2H). |
| | 1-(4-chlorophenyl)-3-(4- | |
| | methoxyphenyl)-7-((2,2,2- | |
| | trifluoroethyl)amino)-3,4- | |
| | dihydropyrimido[4,5-d]pyrimidin- | |
| | 2(1H)-one | |
| Example 253 (via Step F) | 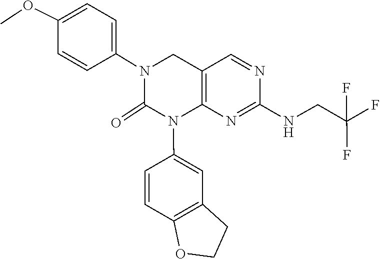 [SMILES: COc1ccc(N2Cc3cnc(NCC(F)(F)F)nc3N(c3ccc4c(c3)CCO4)C2=O)cc1] | LC-MS (ESI): m/z 472.1 [M + H]⁺. 1H NMR (400 MHz, Chloroform-d) δ 7.84 (s, 1H), 7.28 (d, J = 9.0 Hz, 2H), 7.07 (d, J = 2.1 Hz, 1H), 7.02- 6.89 (m, 3H), 6.84 (d, J = 8.4 Hz, 1H), 4.76 (s, 2H), 4.63 (t, J = 8.7 Hz, 2H), 3.82 (s, 5H), 3.23 (t, J = 8.7 Hz, 2H). |
| | 1-(2,3-dihydrobenzofuran-5-yl)-3- | |
| | (4-methoxyphenyl)-7-((2,2,2- | |
| | trifluoroethyl)amino)-3,4- | |
| | dihydropyrimido[4,5-d]pyrimidin | |
| | 2(1H)-one | |
| Example 254 (via Step F) | 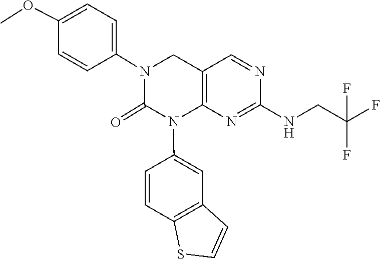 [SMILES: COc1ccc(N2Cc3cnc(NCC(F)(F)F)nc3N(c3ccc4sccc4c3)C2=O)cc1] | LC-MS (ESI): m/z 486.1 [M + H]⁺. 1H NMR (400 MHz, Chloroform-d) δ 7.95 (d, J = 8.6 Hz, 1H), 7.89 (s, 1H), 7.75 (d, J = 2.0 Hz, 1H), 7.51 (d, J = 5.5 Hz, 1H), 7.34 (dd, J = 5.5, 0.7 Hz, 1H), 7.30 (d, J = 9.0 Hz, 2H), 7.23 (dd, J = 8.6, 2.0 Hz, 1H), 6.94 (d, J = 9.0 Hz, 2H), 4.81 (s, 2H), 3.82 (s, 3H), 3.78 (brs, 2H). |
| | 1-(benzo[b]thiophen-5-yl)-3-(4- | |
| | methoxyphenyl)-7-((2,2,2- | |
| | trifluoroethyl)amino)-3,4- | |
| | dihydropyrimido[4,5-d]pyrimidin | |
| | 2(1H)-one | |
| Example 255 (via Step F) | 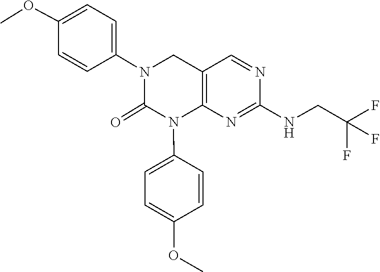 [SMILES: COc1ccc(N2Cc3cnc(NCC(F)(F)F)nc3N(c3ccc(OC)cc3)C2=O)cc1] | LC-MS (ESI): m/z 460.1 [M + H]⁺ 1H NMR (400 MHz, Chloroform-d) δ 7.98 (s, 1H), 7.29 (d, J = 8.9 Hz, 2H), 7.19 (d, J = 8.9 Hz, 2H), 6.96 (d, J = 8.9 Hz, 2H), 6.92 (d, J = 9.0 Hz, 2H), 5.13 (brs, 1H), 4.75 (s, 2H), 3.59 (brs, 2H), 3.84 (s, 3H), 3.80 (s, 3H). |
| | 1,3-bis(4-methoxyphenyl)-7- | |
| | ((2,2,2-trifluoroethyl)amino)-3,4- | |
| | dihydropyrimido[4,5-d]pyrimidin- | |
| | 2(1H)-one | |
| Example 256 (via Step F) | 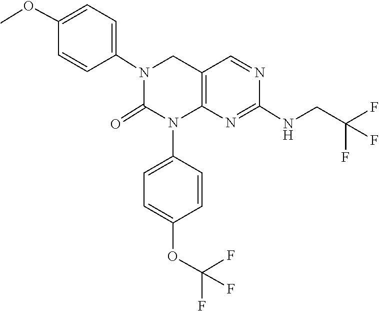 [SMILES: COc1ccc(N2Cc3cnc(NCC(F)(F)F)nc3N(c3ccc(OC(F)(F)F)cc3)C2=O)cc1] | LC-MS (ESI): m/z 514.1 [M + H]⁺. 1H NMR (400 MHz, Chloroform-d) δ 8.01 (s, 1H), 7.35 − 7.27 (m, 6H), 6.93 (d, J = 9.0 Hz, 2H), 5.17 (brs, 1H), 4.77 (s, 2H), 3.83 (brs, 2H), 3.81 (s, 3H). |
| | 3-(4-methoxyphenyl)-7-((2,2,2- | |
| | trifluoroethyl)amino)-1-(4- | |
| | (trifluoromethoxy)phenyl)-3,4- | |
| | dihydropyrimido[4,5-d]pyrimidin- | |
| | 2(1H)-one | |
| Example 425 | 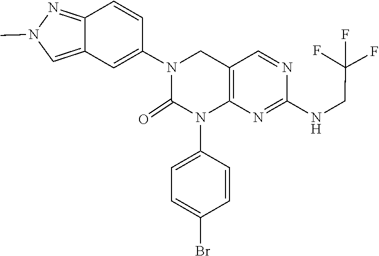 [SMILES: Cn1cc2cc(N3Cc4cnc(NCC(F)(F)F)nc4N(c4ccc(Br)cc4)C3=O)ccc2n1] | LC-MS: m/z 533 (M + H)+. 1H NMR (400 MHz, DMSO-d₆) δ: 8.36 (s, 1H), 8.14 (s, 1H), 7.70 (d, J = 1.4 Hz, 1H), 7.64 (d, J = 8.6 Hz, 2H), 7.58 (d, J = 9.2 Hz, 2H), 7.33- 7.24 (m, 3H), 4.84 (s, 2H), 4.17 (s, 3H), 4.04-3.62 (m, 2H). |
| | 1-(4-bromophenyl)-3-(2-methyl- | |
| | 2H-indazol-5-yl)-7-((2,2,2- | |
| | trifluoroethyl)amino)-3,4- | |
| | dihydropyrimido[4,5-d]pyrimidin- | |
| | 2(1H)-one | |

### General Procedure VII

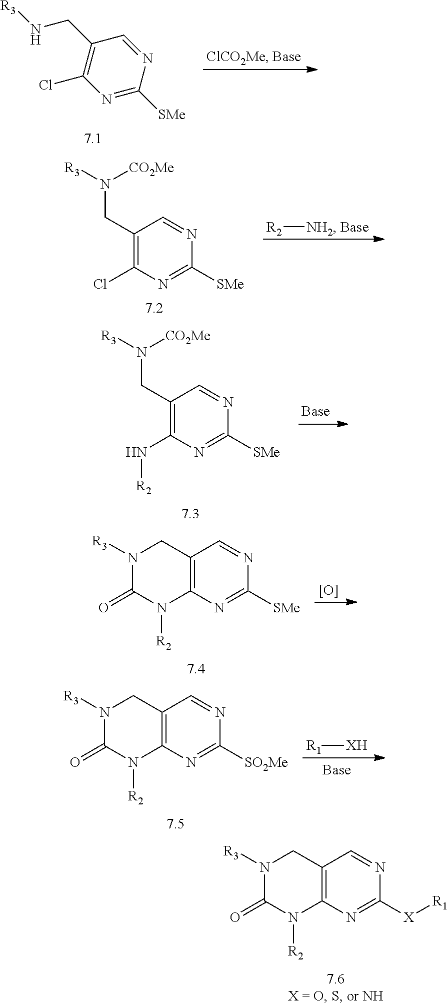

Compounds of structure 7.6 were obtained through the scheme depicted as General Procedure VII. Amine 7.1 (obtained from General Procedure VII, Step A) was reacted with methyl chloroformate to generate carbamate 7.2. The desired R₂ group was introduced using a base mediated aromatic substitution reaction to generate compound 7.3. Compound 7.3 was converted to cyclic urea 7.4 using a base mediated cyclization. Aryl thiol 7.4 was then oxidized to sulfone 7.5, and the desired R₁ group was introduced using a base mediated aromatic substitution reaction to afford compound 7.6.

### Preparation of Example 257 Via General Procedure VII

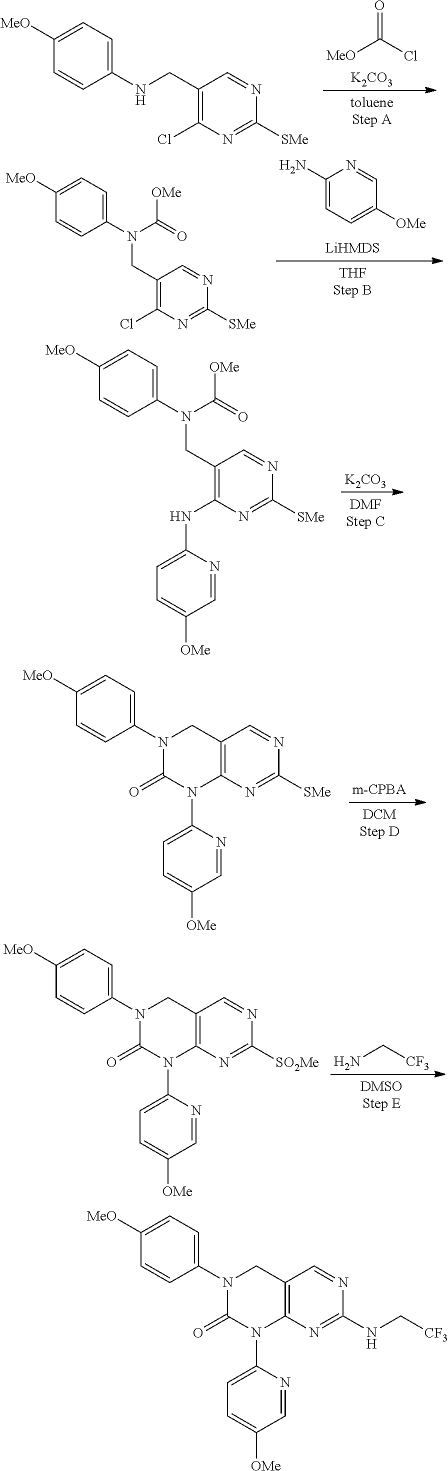

### Step A: methyl (4-chloro-2-(methylthio)pyrimidin-5-yl)methyl(4-methoxyphenyl)carbamate

To a solution of N-((4-chloro-2-(methylthio)pyrimidin-5-yl)methyl)-4-methoxyaniline (500 mg, 1.7 mmol, 1.0 eq., obtained from General Procedure VI, Step A) and K₂CO₃ (701 mg, 5.1 mmol, 3.0 eq.) in toluene (20 mL) was added methyl carbonochloridate (240 mg, 2.6 mmol, 1.5 eq.) at 0° C. via a syringe. The resulting solution was stirred at room temperature overnight. Then the reaction mixture was treated with ice water (10 mL), extracted with EtOAc (10 mL×3), the combined organic layers were washed with brine (20 mL) and dried over Na₂SO₄, concentrated under reduced pressure, the residue was purified by flash column chromatography in silica gel to afford methyl (4-chloro-2-(methylthio)pyrimidin-5-yl)methyl(4-methoxyphenyl)carbamate (580 mg, 97% yield) as a yellow solid. LC-MS (ESI): m/z 354 [M+H]⁺.

### Step B: methyl-4-methoxyphenyl((4-(5-methoxypyridin-2-ylamino)-2-(methylthio)pyrimidin-5-yl)methyl)carbamate

To a solution of methyl (4-chloro-2-(methylthio)pyrimidin-5-yl)methyl(4-methoxyphenyl)carbamate (300 mg, 0.8 mmol, 1.0 eq.) and 5-methoxypyridin-2-amine (158 mg, 1.3 mmol, 1.6 eq.) in THF (10 mL), was added LiHMDS (1.0 M in THF, 2.4 mL, 2.4 mmol, 3.0 eq.) at −65° C. via a syringe over 10 min. After addition, the reaction mixture was allowed to warm to room temperature and stirred for additional 4 hrs. Then the reaction was quenched with H₂O (15 mL), extracted with EtOAc (20 mL×3), the combined organic layers were washed with brine (30 mL) and dried over Na₂SO₄, concentrated under reduced pressure, the residue was purified by flash column chromatography on silica gel to give methyl-4-methoxyphenyl((4-(5-methoxypyridin-2-ylamino)-2-(methylthio)pyrimidin-5-yl)methyl)carbamate (135 mg, 36% yield) as a yellow solid. LC-MS (ESI): m/z 442 [M+H]⁺.

### Step C: 3-(4-methoxyphenyl)-1-(5-methoxypyridin-2-yl)-7-(methylthio)-3,4-dihydropyrimido[4,5-d]pyrimidin-2(1H)-one

Methyl-4-methoxyphenyl((4-(5-methoxypyridin-2-ylamino)-2-(methylthio)pyrimidin-5-yl)methyl)carbamate (135 mg, 0.3 mmol, 1.0 eq.) and K₂CO₃ (2.1 g, 15.3 mmol, 51.0 eq.) in DMF (20 mL). The mixture was stirred at 130° C. for 48 hrs. the reaction mixture was concentrated under reduced pressure, the residue was treated with H₂O (20 mL), extracted with EtOAc (30 mL×3), the combined organic layers were washed with brine (20 mL) and dried over Na₂SO₄ and concentrated under reduced pressure, the residue was purified by flash column chromatography on silica gel to give 3-(4-methoxyphenyl)-1-(5-methoxypyridin-2-yl)-7-(methylthio)-3,4-dihydropyrimido[4,5-d]pyrimidin-2(1H)-one (100 mg, 80% yield) as a yellow solid. LC-MS (ESI): m/z 410 [M+H]⁺.

3-(4-Methoxyphenyl)-1-(5-methoxypyridin-2-yl)-7-(2,2,2-trifluoroethylamino)-3,4-dihydropyrimido[4,5-d]pyrimidin-2(1H)-one (Example 257) was synthesized from 3-(4-methoxyphenyl)-1-(5-methoxypyridin-2-yl)-7-(methylthio)-3,4-dihydropyrimido[4,5-d]pyrimidin-2(1H)-one and 2,2,2-trifluoroethanamine via general procedure IV (Step D, E).

¹H NMR (400 MHz, CDCl₃) δ (ppm): 8.28 (d, J=2.4 Hz, 1H), 7.99 (s, 1H), 7.40-7.27 (m, 4H), 6.92 (d, J=8.7 Hz, 2H), 5.25 (br s, 1H), 4.76 (s, 2H), 4.01-3.71 (m, 2H, overlapped), 3.91 (s, 3H), 3.81 (s, 3H).

LC-MS (ESI): m/z 461 [M+H]⁺.

The procedure set forth above for General Procedure VII was used to synthesize the following compounds by using appropriate starting materials:

| Cpd No. | Structure | Characterization |
|---|---|---|
| Example 258 | 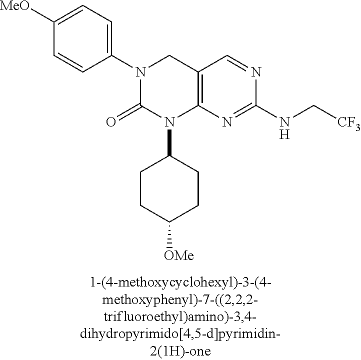 | LC-MS (ESI): m/z 466.2 [M + H]+. 1H NMR (400 MHz, CDCl3) δ: 7.90 (s, 1H), 7.21 (d, J = 8.8 Hz, 2H), 6.92 (d, J = 9.2 Hz, 2H), 5.34 (bs, 1H), 4.70-4.62 (m, 1H), 4.54 (s, 2H), 4.18-4.09 (m, 2H), 3.81 (s, 3H), 3.36 (s, 3H), 3.21- 3.16 (m, 1H), 2.63-2.53 (m, 2H), 2.18-2.15 (m, 2H), 1.82-1.79 (m, 2H), 1.38-1.27 (m, 2H). |
| Example 259 | 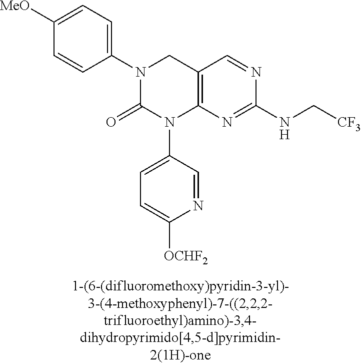 [SMILES: COc1ccc(N2Cc3cnc(NCC(F)(F)F)nc3N(c3ccc(OC(F)F)nc3)C2=O)cc1] | LC-MS (ESI): m/z 497.1 [M + H]+. 1H NMR (400 MHz, DMSO-d₆) δ 8.23 (d, J = 2.0 Hz, 1H), 8.15 (s, 1H), 7.93 (dd, J = 8.8 Hz, 2.4 Hz, 1H), 7.67 (d, J_{HF} = 72.4 Hz, 1H), 7.55 (bs, 1H), 7.34 (d, J = 8.8 Hz, 2H), 7.19 (d, J = 8.8 Hz, 1H), 6.97 (d, J = 8.8 Hz, 2H), 4.78 (s, 2H), 4.05-3.53 (m, 2H), 3.77 (s, 3H). |
| Example 260 | 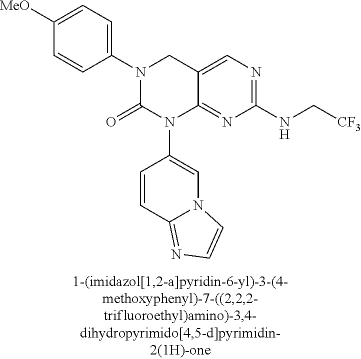 [SMILES: COc1ccc(N2Cc3cnc(NCC(F)(F)F)nc3N(c3ccc4nccn4c3)C2=O)cc1] | LC-MS (ESI): m/z 470.1 [M + H]+. 1H NMR (400 MHz, CDCl3) δ: 8.14 (s, 1H), 7.97 (s, 1H), 7.60- 7.52 (m, 3H), 7.21 (d, J = 8.8 Hz, 2H), 7.04 (d, J = 9.2 Hz, 1H), 6.87 (d, J = 8.8 Hz, 2H), 5.19 (s, 1H), 4.71 (s, 2H), 4.04-3.55 (m, 2H), 3.75 (s, 3H). |
| Example 261 | 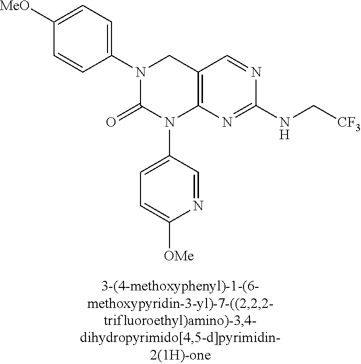 [SMILES: COc1ccc(N2Cc3cnc(NCC(F)(F)F)nc3N(c3ccc(OC)nc3)C2=O)cc1] | LC-MS (ESI): m/z 461.0 [M + H]+. 1H NMR (400 MHz, DMSO-d₆) δ: 8.13 (s, 1H), 8.09 (d, J = 2.4 Hz, 1H), 7.67 (dd, J = 8.4 Hz, 2.4 Hz, 1H), 7.53 (bs, 1H), 7.35 (d, J = 9.2 Hz, 2H), 6.97 (d, J = 9.2 Hz, 2H), 6.90 (d, J = 8.8 Hz, 1H), 4.77 (s, 2H), 4.11-3.78 (m, 2H), 3.89 (s, 3H), 3.77 (s, 3H). |

### General Procedure VIII

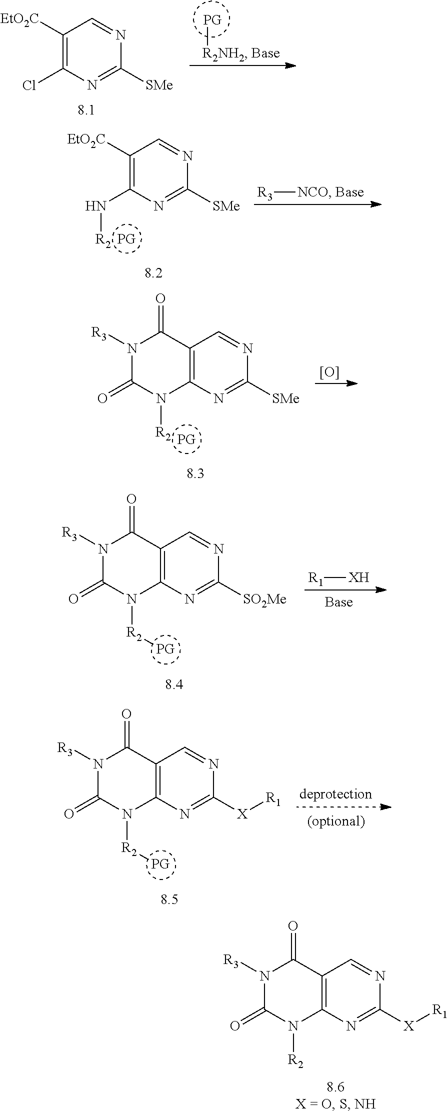

Compounds of structure 8.6 were obtained through the scheme depicted as General Procedure VIII. Beginning with aryl chloride 8.1, the desired R₂ group (optionally protected) was introduced using a base mediated aromatic substitution reaction to generate heteroaryl amine 8.2. The desired R₃ group was introduced by reacting amino-ester 8.2 with the appropriate isocyanate under basic conditions to form cyclic urea 8.3. Aryl thiol 8.3 was oxidized to sulfone 8.4 and the desired R₁ group was introduced using a base mediated aromatic substitution reaction to afford compound 8.5. If necessary, compound 8.5 was then deprotected to afford compound 8.6.

### Preparation of Example 262 Via General Procedure VIII

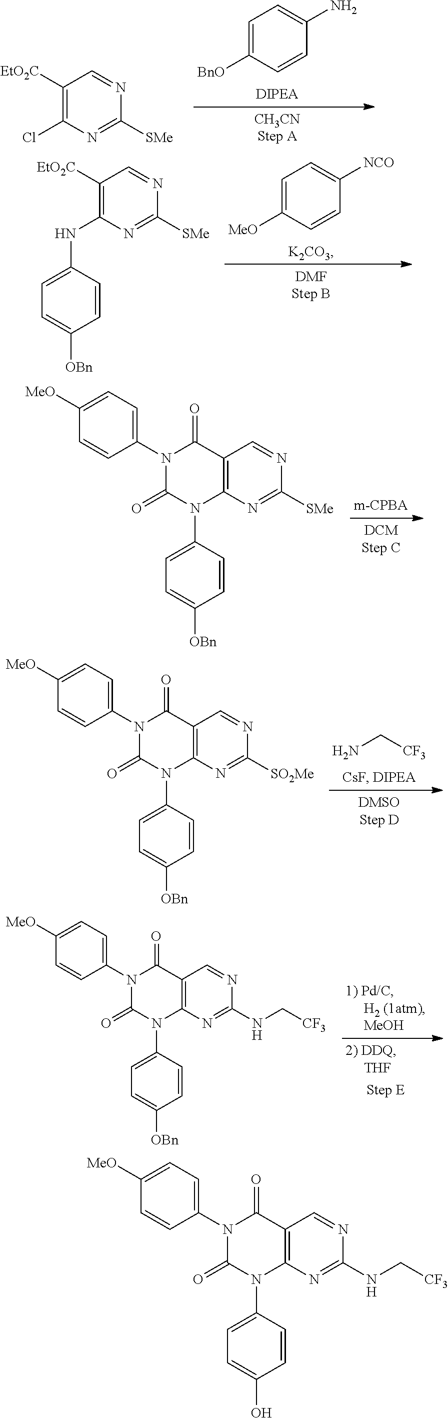

### Step A: ethyl 4-((4-(benzyloxy)phenyl)amino)-2-(methylthio)pyrimidine-5-carboxylate

To a solution of ethyl 4-chloro-2-(methylthio)pyrimidine-5-carboxylate (1.0 g, 4.3 mmol, 1.0 eq.) in DMSO (10 mL) was added 4-(benzyloxy)aniline (950 mg, 4.7 mmol, 1.1 eq.) and DIPEA (1.6 g, 12.9 mmol, 3.0 eq.) at room temperature. The reaction mixture was stirred at 80° C. for 2 hrs. Then the reaction mixture was quenched with ice water (50 mL) and extracted with EtOAc (50 mL×3). The combined organic layers were washed with brine (30 mL), dried over Na₂SO₄ and concentrated under reduced pressure, the residue was purified by flash column chromatography on silica gel to give ethyl 4-((4-(benzyloxy)phenyl)amino)-2-(methylthio)pyrimidine-5-carboxylate (1.6 g, 95% yield) as a white solid. LC-MS (ESI): m/z 396 [M+H]⁺.

### Step B: 1-(4-(benzyloxy)phenyl)-3-(4-methoxyphenyl)-7-(methylthio)pyrimido[4,5-d]pyrimidine-2,4(1H,3H)-dione

To a solution of ethyl 4-((4-(benzyloxy)phenyl)amino)-2-(methylthio)pyrimidine-5-carboxylate (1.0 g, 2.5 mmol, 1.0 eq.) in DMF (10 mL) was added 1-isocyanato-4-methoxybenzene (560 mg, 3.7 mmol, 1.5 eq.) and K₂CO₃ (690 mg, 5.0 mmol, 2.0 eq.) at room temperature. The resulting mixture was stirred for 16 hrs. Then the reaction mixture was quenched with ice water (10 mL) and extracted with EtOAc (20 mL×3). The combined organic layers were washed with brine (30 mL), dried over Na₂SO₄ and concentrated under reduced pressure, the residue was purified by flash column chromatography to afford 1-(4-(benzyloxy)phenyl)-3-(4-methoxyphenyl)-7-(methylthio)pyrimido[4,5-d]pyrimidine-2,4(1H,3H)-dione (300 mg, 24% yield) as a white solid. LC-MS (ESI): m/z 499 [M+H]⁺.

### Step C: 1-(4-(benzyloxy)phenyl)-3-(4-methoxyphenyl)-7-(methylsulfonyl)pyrimido[4,5-d]pyrimidine-2,4(1H,3H)-dione

To a solution of 1-(4-(benzyloxy)phenyl)-3-(4-methoxyphenyl)-7-(methylthio)pyrimido[4,5-d]pyrimidine-2,4(1H,3H)-dione (300 mg, 0.6 mmol, 1.0 eq.) in DCM (10 mL) was added m-CPBA (310 mg, 1.8 mmol, 3.0 eq.) in several portions at room temperature. The resulting mixture was stirred for 2 hrs. Then the reaction mixture was quenched with NaHSO₃ (sat. aq.) (10 mL), extracted with DCM (20 mL×2). The combined organic layers were washed with brine (30 mL), dried over Na₂SO₄ and concentrated under reduced pressure, the residue was purified by column chromatography to afford 1-(4-(benzyloxy)phenyl)-3-(4-methoxyphenyl)-7-(methylsulfonyl)pyrimido[4,5-d]pyrimidine-2,4(1H,3H)-dione (260 mg, 82% yield) as a white solid. LC-MS (ESI): m/z 531 [M+H]⁺.

### Step D: 1-(4-(benzyloxy)phenyl)-3-(4-methoxyphenyl)-7-((2,2,2-trifluoroethyl)amino)pyrimido[4,5-d]pyrimidine-2,4(1H,3H)-dione

To a solution of 1-(4-(benzyloxy)phenyl)-3-(4-methoxyphenyl)-7-(methylsulfonyl)pyrimido[4,5-d]pyrimidine-2,4(1H,3H)-dione (260 mg, 0.5 mmol, 1.0 eq.) in DMSO (5 mL) was added CsF (74 mg, 0.5 mmol, 1.0 eq.), DIPEA (194 mg, 1.5 mmol, 3.0 eq.) and 2,2,2-trifluoroethanamine (150 mg, 1.5 mmol, 3.0 eq.) at room temperature. The resulting mixture was stirred at 80° C. for 16 hrs in a sealed tube. Then the reaction mixture was quenched with ice water (30 mL) and extracted with DCM (30 mL×3). The combined organic layers were washed with brine (30 mL), dried over Na₂SO₄ and concentrated under reduced pressure, the residue was purified by flash column chromatography on silica gel to give 1-(4-(benzyloxy)phenyl)-3-(4-methoxyphenyl)-7-((2,2,2-trifluoroethyl)amino)pyrimido[4,5-d]pyrimidine-2,4(1H,3H)-dione (100 mg, 36% yield) as a white solid. LC-MS (ESI): m/z 550 [M+H]⁺.

### Step E: 1-(4-hydroxyphenyl)-3-(4-methoxyphenyl)-7-((2,2,2-trifluoroethyl)amino)pyrimido[4,5-d]pyrimidine-2,4(1H,3H)-dione

To a solution of 1-(4-(benzyloxy)phenyl)-3-(4-methoxyphenyl)-7-((2,2,2-trifluoroethyl)amino)pyrimido[4,5-d]pyrimidine-2,4(1H,3H)-dione (50 mg, 0.1 mmol, 1.0 eq.) in MeOH (5 mL) was added 5% wt Pd/C (10% w/w, 5 mg) at room temperature. And the reaction mixture was stirred at room temperature for 16 hrs under H₂ atmosphere (1 atm). After completion, the catalyst was removed by filtration, and the filtrate was concentrated under reduced pressure. (crude LC-MS showed that ˜30% over-reduced adduct (M+2+H)⁺ was formed), then the crude mixture wad re-dissolved in anhy. THF (5 mL), and DDQ (45 mg) was added in one portion, the resulting mixture was stirred at room temperature for additional 2 hrs. Then the reaction mixture was quenched with NaHSO₃ (sat.aq.) (10 mL), the resulting mixture was extracted with EtOAc (10 mL×3). The combined organic layers were washed with brine (10 mL), dried over Na₂SO₄ and concentrated under reduced pressure. The crude residue was purified by RP-prep-HPLC to afford 1-(4-hydroxyphenyl)-3-(4-methoxyphenyl)-7-((2,2,2-trifluoroethyl)amino)pyrimido[4,5-d]pyrimidine-2,4(1H,3H)-dione (Example 262).

¹H NMR (400 MHz, DMSO-d₆) (mixture of two tautomers depicted below, ratio: ca. 1:1) δ (ppm): 9.69 (m, 1H, overlapped), 8.87 and 8.82 (two sets of s, 1H in total), 8.75-8.70 and 8.50-8.45 (two sets of m, 1H in total), 7.33-7.03 (multiple of doublet peaks, 6H), 6.91-6.78 (m, 2H), 4.24-4.08 (m, 1H), 3.85-3.75 (m, 1H), 3.78 (s, 3H, overlapped).

LC-MS (ESI): m/z 460 [M+H]⁺.

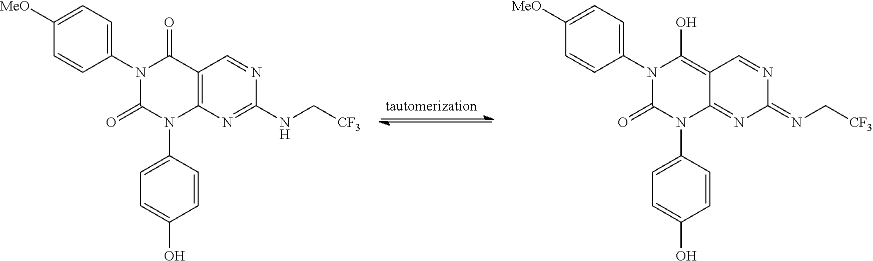 [SMILES: COc1ccc(-n2c(=O)c3cnc(NCC(F)(F)F)nc3n(-c3ccc(O)cc3)c2=O)cc1 ; COc1ccc(-n2c(O)c3cn/c(=N\CC(F)(F)F)nc-3n(-c3ccc(O)cc3)c2=O)cc1]

### General Procedure IX

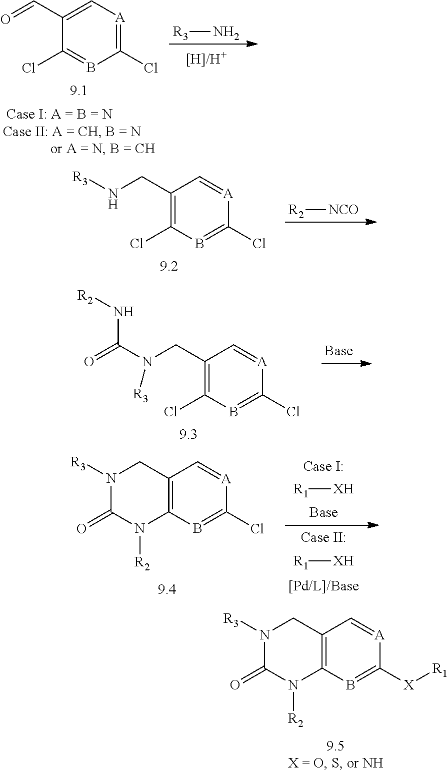

Compounds of structure 9.5 were obtained through the scheme depicted as General Procedure IX. Beginning with aldehyde 9.1, the desired R₃ group was introduced using a reductive amination to generate amine 9.2. The desired R₃ group was introduced by reacting amine 9.2 with the appropriate isocyanate to form acyclic urea 9.3. Compound 9.3 was then subjected to basic conditions to form cyclic urea 9.4. The desired R₁ group was then introduced either through a base mediated nucleophilic substitution (Case I) or a palladium mediated C—X coupling (Case II) to afford compound 9.5.

### Preparation of Example 263 Via General Procedure IX (Case I)

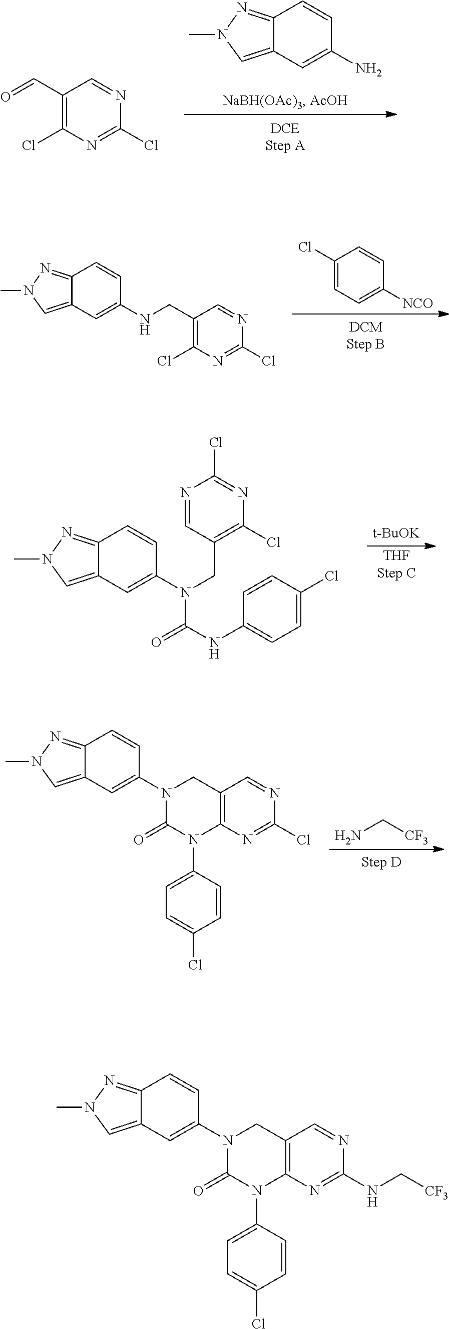

### Step A: 3-(4-chlorophenyl)-1-((2,4-dichloropyrimidin-5-yl)methyl)-1-(2-methyl-2H-indazol-5-yl)urea

2,4-dichloropyrimidine-5-carbaldehyde (500 mg, 2.83 mmol, 1.0 eq.) was dissolved in DCM (7 ml), and then acetic acid (485 μl, 8.48 mmol, 3.0 eq.) was added. The mixture was cooled to 0° C. before the 2-methyl-2H-indazol-5-amine (416 mg, 2.83 mmol, 1.0 eq.) was added. At 0° C., sodium triacetoxyhydroborate (898 mg, 4.24 mmol, 2.0 eq.) was added as one portion. After complete reaction as monitored by LC-MS (m/z 308 [M+H]⁺ was detected), the reaction mixture was used directly for the Step B.

### Step B: 3-(4-chlorophenyl)-1-((2,4-dichloropyrimidin-5-yl)methyl)-1-(2-methyl-2H-indazol-5-yl)urea

1-chloro-4-isocyanatobenzene (434 mg, 2.83 mmol, 2.0 eq.) was added to crude 3-(4-chlorophenyl)-1-((2,4-dichloropyrimidin-5-yl)methyl)-1-(2-methyl-2H-indazol-5-yl)urea (Crude material, 435 mg, 1.4 mmol, 1.0 eq.) of the reaction mixture from Step A. After 30 mins, silica gel was added to the reaction mixture at the sample was concentrated to dryness. The crude solid was dry-loaded and purified by column chromatography to afford 3-(4-chlorophenyl)-1-((2,4-dichloropyrimidin-5-yl)methyl)-1-(2-methyl-2H-indazol-5-yl)urea (366.2 mg, 0.793 mmol, 56.1% yield). LC-MS (ESI): m/z 461 [M+H]⁺.

### Step C: 7-chloro-1-(4-chlorophenyl)-3-(2-methyl-2H-indazol-5-yl)-3,4-dihydropyrimido[4,5-d]pyrimidin-2(1H)-one

3-(4-chlorophenyl)-1-((2,4-dichloropyrimidin-5-yl)methyl)-1-(2-methyl-2H-indazol-5-yl)urea (330.3 mg, 0.715 mmol, 1.0 eq.) was dissolved in THF and cooled to 0° C., then KO^{t}Bu (96 mg, 0.858 mmol, 1.2 eq.) was added. The mixture was allowed to warm to rt. After stirring 15 mins, another 0.2 eq KO^{t}Bu was added and the reaction mixture was heated to 60° C. After 20 mins of heating, the reaction was complete. Silica gel was added to the reaction mixture, which was then concentrated under reduced pressure. The crude solid was dry-loaded and purified by column chromatography to afford 7-chloro-1-(4-chlorophenyl)-3-(2-methyl-2H-indazol-5-yl)-3,4-dihydropyrimido[4,5-d]pyrimidin-2(1H)-one was collected and concentrated as product (248.7 mg, 0.585 mmol, 82% yield). LC-MS (ESI): m/z 425 [M+H]⁺.

### Step D: 1-(4-chlorophenyl)-3-(2-methyl-2H-indazol-5-yl)-7-((2,2,2-trifluoroethyl)amino)-3,4-dihydropyrimido[4,5-d]pyrimidin-2(1H)-one

7-chloro-1-(4-chlorophenyl)-3-(2-methyl-2H-indazol-5-yl)-3,4-dihydropyrimido[4,5-d]pyrimidin-2(1H)-one (50 mg, 0.118 mmol, 1.0 eq.) was dissolved in DMSO (392 μl), cesium fluoride (35.7 mg, 0.235 mmol, 2.0 eq.), DIPEA (41.1 μl, 0.235 mmol, 2.0 eq.) and 2,2,2-trifluoroethan-1-amine (46.1 μl, 0.588 mmol, 4.0 eq.) were added quickly. Then the reaction was sealed and heated to 110° C. for 5h. The reaction mixture was then diluted by ethyl acetate and washed with DI water. The organic layer was collected and dried over sodium sulfate. After filtration, the solution was concentrated with silica gel, then dry-loaded and purified by column chromatography to afford 1-(4-chlorophenyl)-3-(2-methyl-2H-indazol-5-yl)-7-((2,2,2-trifluoroethyl)amino)-3,4-dihydropyrimido[4,5-d]pyrimidin-2(1H)-one (Example 263).

1H NMR (400 MHz, Chloroform-d) δ 8.02 (s, 1H), 7.89 (s, 1H), 7.71 (d, J=9.2 Hz, 1H), 7.61 (s, 1H), 7.42 (d, J=8.6 Hz, 2H), 7.28-7.24 (m, 3H), 5.16 (brs, 1H), 4.83 (s, 2H), 4.22 (s, 3H), 3.90 (brs, 2H). LC-MS (ESI): m/z 488.0 [M+H]⁺.

### Preparation of Example 264 via General Procedure IX (Case II)

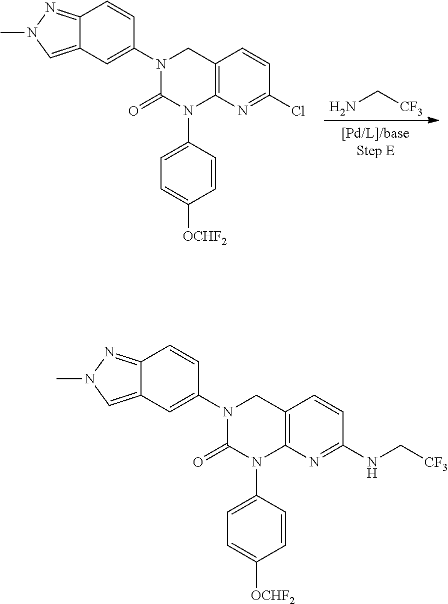 [SMILES: Cn1cc2cc(N3Cc4ccc(Cl)nc4N(c4ccc(OC(F)F)cc4)C3=O)ccc2n1 ; NCC(F)(F)F ; Cn1cc2cc(N3Cc4ccc(NCC(F)(F)F)nc4N(c4ccc(OC(F)F)cc4)C3=O)ccc2n1]

### Step E: 1-(4-(difluoromethoxy)phenyl)-3-(2-methyl-2H-indazol-5-yl)-7-((2,2,2-trifluoroethyl)amino)-3,4-dihydropyrido[2,3-d]pyrimidin-2(1H)-one

7-chloro-1-(4-(difluoromethoxy)phenyl)-3-(2-methyl-2H-indazol-5-yl)-3,4-dihydropyrido[2,3-d]pyrimidin-2(1H)-one (50 mg, 0.110 mmol, 1.0 eq.), tris(dibenzylideneacetone)dipalladium(0) chloroform adduct (5.68 mg, 5.48 μmol, 0.05 eq., obtained from general procedure IX, Steps A-C), sodium tert-butoxide (15.81 mg, 0.165 mmol, 1.5 eq.) and dicyclohexyl(2′,4′,6′-triisopropyl-[1,1′-biphenyl]-2-yl)phosphane (5.23 mg, 10.97 μmol, 0.1 eq.) were dissolved in Dioxane (366 μl). 2,2,2-trifluoroethan-1-amine (43.0 μl, 0.548 mmol, 5.0 eq.) was added to the resulting mixture. The vial was sealed with a cap and heated to 100° C. for 3h. Silica gel was added directly to the crude reaction mixture, which was then concentrated, dry-loaded and purified by column chromatography to afford 1-(4-(difluoromethoxy)phenyl)-3-(2-methyl-2H-indazol-5-yl)-7-((2,2,2-trifluoroethyl)amino)-3,4-dihydropyrido[2,3-d]pyrimidin-2(1H)-one (Example 264).

1H NMR (400 MHz, DMSO-d₆) δ 8.33 (s, 1H), 7.67 (s, 1H), 7.56 (d, J=9.1 Hz, 1H), 7.37 (d, J=8.2 Hz, 1H), 7.32 (d, J=8.4 Hz, 2H), 7.28 (t, JH-F=63.6 Hz 1H), 7.25 (s, 1H), 7.21 (d, J=8.4 Hz, 2H), 7.09-7.03 (m, 1H), 6.24 (d, J=8.2 Hz, 1H), 4.82 (s, 2H), 4.16 (s, 3H), 3.67 (p, J=9.6 Hz, 2H).

LC-MS (ESI): m/z 519.1 [M+H]⁺.

The procedure set forth above for General Procedure IX was used to synthesize the following compounds by using appropriate starting materials:

| Cpd No. | Structure | Characterization |
|---|---|---|
| Example 265 (Case I) | 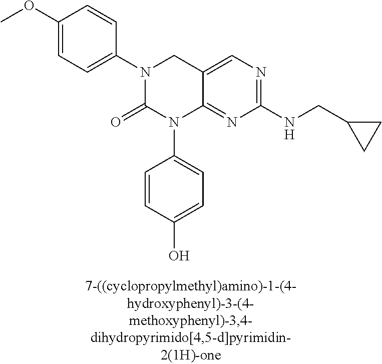 [SMILES: COc1ccc(N2Cc3cnc(NCC4CC4)nc3N(c3ccc(O)cc3)C2=O)cc1] | LC-MS (ESI): m/z 417.8 [M + H]+. 1H NMR (400 MHz, Chloroform-d) δ 7.93 (s, 1H), 7.29 (d, J = 9.0 Hz, 2H), 7.05 (d, J = 8.7 Hz, 2H), 6.93 (d, J = 9.0 Hz, 2H), 6.71 (d, J = 8.7 Hz, 2H), 5.09 (t, J = 5.6 Hz, 1H), 4.71 (s, 2H), 3.81 (s, 3H), 3.02 (br s, 2H), 0.93-0.86 (m, 1H), 0.41 (d, J = 7.7 Hz, 2H), 0.10 (br s, 2H). |
| Example 266 (Case I) | 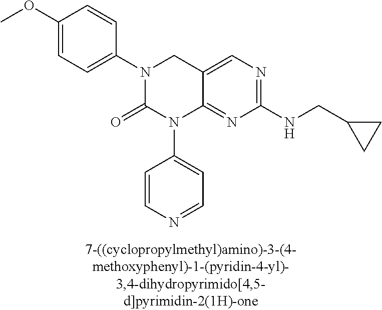 [SMILES: COc1ccc(N2Cc3cnc(NCC4CC4)nc3N(c3ccncc3)C2=O)cc1] | LC-MS (ESI): m/z 402.8 [M + H]+. 1H NMR (400 MHz, Chloroform-d) δ 8.78-8.60 (m, 2H), 7.98 (s, 1H), 7.32-7.29 (m, 2H), 7.28 (d, J = 9.0 Hz, 2H), 6.93 (d, J = 9.0 Hz, 2H), 5.08 (s, 1H), 4.74 (s, 2H), 3.81 (s, 3H), 3.01 (brs, 2H), 0.92 (brs, 1H), 0.44 (brs, 2H), 0.11 (brs, 2H). |
| Example 267 (Case I) | 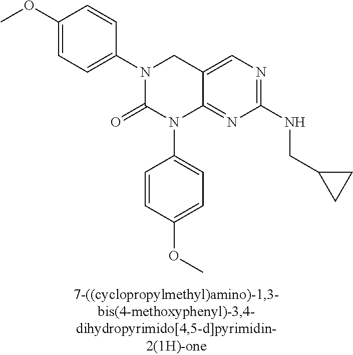 [SMILES: COc1ccc(N2Cc3cnc(NCC4CC4)nc3N(c3ccc(OC)cc3)C2=O)cc1] | LC-MS (ESI): m/z 431.9 [M + H]+. 1H NMR (400 MHz, Chloroform-d) δ 7.93 (s, 1H), 7.28 (d, J = 9.0 Hz, 2H), 7.20 (d, J = 8.9 Hz, 2H), 6.96 (d, J = 8.9 Hz, 2H), 6.91 (d, J = 8.9 Hz, 2H), 5.08 (brs, 1H), 4.71 (s, 2H), 3.84 (s, 3H), 3.80 (s, 3H), 3.05 (s, 2H), 1.00-0.78 (m, 1H), 0.43 (d, J = 7.7 Hz, 2H), 0.11 (s, 2H). |
| Example 268 (Case II) | 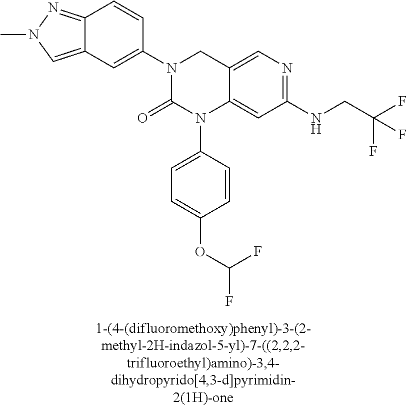 [SMILES: Cn1cc2cc(N3Cc4cnc(NCC(F)(F)F)cc4N(c4ccc(OC(F)F)cc4)C3=O)ccc2n1] | LC-MS (ESI): m/z 519.1 [M + H]+. 1H NMR (400 MHz, DMSO-d₆) δ 8.32 (s, 1H), 7.85 (s, 1H), 7.66 (s, 1H), 7.54 (d, J = 9.2 Hz, 1H), 7.43 (d, J = 7.8 Hz, 2H), 7.34 (t, JH-F = 74.6 Hz, 1H), 7.32 (d, J = 7.8 Hz, 3H), 7.25 (d, J = 9.2 Hz, 1H), 7.01 (t, J = 6.4 Hz, 1H), 5.41 (s, 1H), 4.85 (s, 2H), 4.15 (s, 3H), 4.10-4.00 (m, 2H). |
| Example 269 (Case II) | 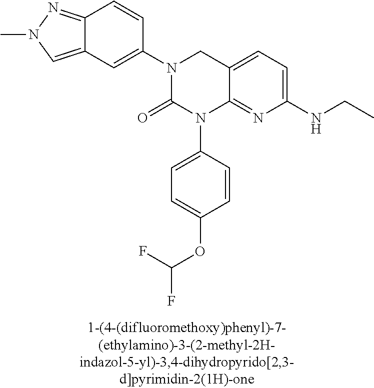 [SMILES: CCNc1ccc2c(n1)N(c1ccc(OC(F)F)cc1)C(=O)N(c1ccc3nn(C)cc3c1)C2] | LC-MS (ESI): m/z 465.1 [M + H]+. 1H NMR (400 MHz, Chloroform-d) δ 7.86 (s, 1H), 7.68 (d, J = 9.2 Hz, 1H), 7.60 (s, 1H), 7.35 (d, J = 8.7 Hz, 2H), 7.30 (d, J = 9.2 Hz, 1H), 7.20- 7.16 (m, 3H), 6.53 (t, JH-F = 74.1 Hz, 1H), 6.01 (d, J = 7.7 Hz, 1H), 4.80 (s, 2H), 4.30 (t, J = 5.7 Hz, 1H), 4.21 (s, 3H), 3.05 (td, J = 7.2, 5.7 Hz, 2H), 1.09 (t, J = 7.2 Hz, 3H). |
| Example 270 (Case II) | 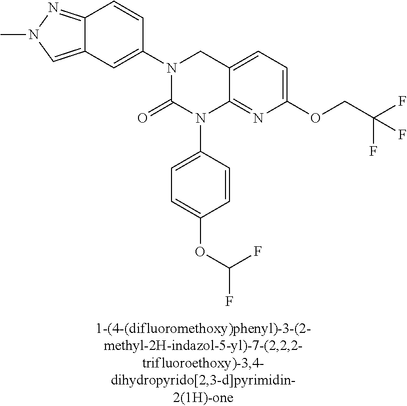 [SMILES: Cn1cc2cc(N3Cc4ccc(OCC(F)(F)F)nc4N(c4ccc(OC(F)F)cc4)C3=O)ccc2n1] | LC-MS (ESI): m/z 520.0 [M + H]+. 1H NMR (400 MHz, Chloroform-d) δ 7.89 (s, 1H), 7.71 (d, J = 9.2 Hz, 1H), 7.62 (s, 1H), 7.42 (d, J = 8.0 Hz, 1H), 7.34 (d, J = 8.6 Hz, 2H), 7.30 (d, J = 9.2, 1H), 7.21 (d, J = 8.6 Hz, 2H), 6.53 (t, JH-F = 73.8 Hz, 1H), 6.50 (d, J = 8.0 Hz, 1H), 4.91 (s, 2H), 4.28 (q, J = 8.5 Hz, 2H), 4.22 (s, 3H). |
| Example 271 (Case II) | 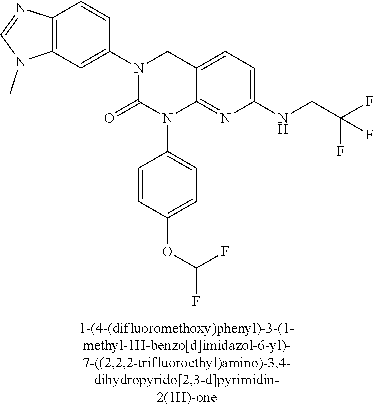 [SMILES: Cn1cnc2ccc(N3Cc4ccc(NCC(F)(F)F)nc4N(c4ccc(OC(F)F)cc4)C3=O)cc21] | LC-MS (ESI): m/z 519.1 [M + H]+. 1H NMR (400 MHz, DMSO-d₆) δ 8.20 (s, 1H), 7.64-7.62 (m, 2H), 7.37 (d, J = 8.1 Hz, 1H), 7.33 (d, J = 8.5 Hz, 2H), 7.26 (t, JH-F = 68.7 Hz, 1H), 7.25 (s, 1H), 7.22 (d, J = 8.5 Hz, 2H), 7.07 (d, J = 4.7 Hz, 1H), 6.25 (d, J = 8.6 Hz, 1H), 4.85 (s, 2H), 3.83 (s, 3H), 3.77-3.60 (m, 2H). |
| Example 272 (Case II) | 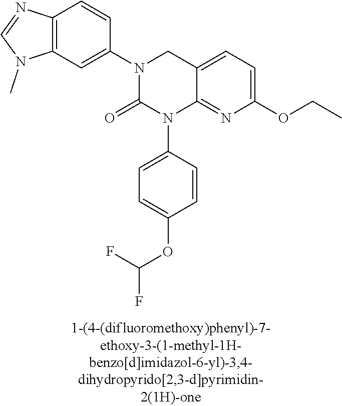 [SMILES: CCOc1ccc2c(n1)N(c1ccc(OC(F)F)cc1)C(=O)N(c1ccc3ncn(C)c3c1)C2] | LC-MS (ESI): m/z 466.1 [M + H]+. 1H NMR (400 MHz, DMSO-d₆) δ 8.20 (s, 1H), 7.66-7.63 (m, 2H), 7.60 (d, J = 8.2 Hz, 1H), 7.40 (d, J =8.4 Hz, 2H), 7.29 (t, JH-F = 71.2 Hz, 1H), 7.28-7.24 (m, 3H), 6.42 (d, J = 7.7 Hz, 1H), 4.94 (s, 2H), 3.91-3.81 (m, 5H), 1.06 (t, J = 7.0 Hz, 3H). |
| Example 273 (Case II) | 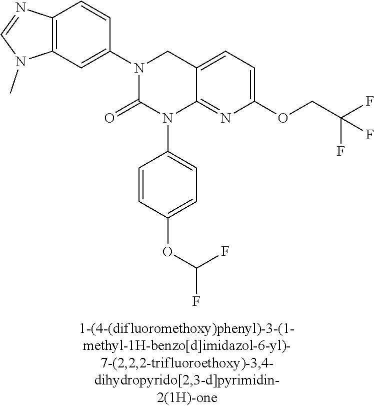 [SMILES: Cn1cnc2ccc(N3Cc4ccc(OCC(F)(F)F)nc4N(c4ccc(OC(F)F)cc4)C3=O)cc21] | LC-MS (ESI): m/z 520.1 [M + H]+. 1H NMR (400 MHz, DMSO-d₆) δ 8.21 (s, 1H), 7.71 (d, J = 8.1 Hz, 1H), 7.67-7.64 (m, 2H), 7.41 (d, J = 8.2, 2H), 7.30-7.24 (m, 4H), 6.61 (d, J = 8.1 Hz, 1H), 4.98 (s, 2H), 4.51 (q, J = 9.1 Hz, 2H), 3.83 (s, 3H). |
| Example 274 (Case II) | 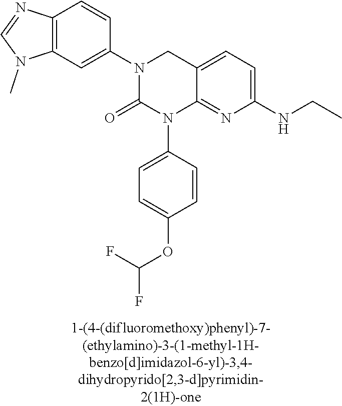 [SMILES: CCNc1ccc2c(n1)N(c1ccc(OC(F)F)cc1)C(=O)N(c1ccc3ncn(C)c3c1)C2] | LC-MS (ESI): m/z 465.1 [M + H]+. 1H NMR (400 MHz, DMSO-d₆) δ 8.17 (s, 1H), 7.62-7.60 (m, 2H), 7.33 (d, J = 8.6 Hz, 2H), 7.30-7.13 (m, 5H), 6.34 (t, J = 5.6 Hz, 1H), 6.06 (d, J = 8.2 Hz, 1H), 4.79 (s, 2H), 3.81 (s, 3H), 2.88- 2.81 (m, 2H), 0.85 (t, J = 7.1 Hz, 3H). |
| Example 275 (Case II) | 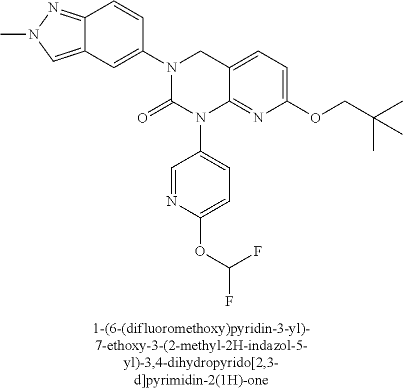 | LC-MS (ESI): m/z 467.1 [M + H]+. 1H NMR (400 MHz, DMSO-d₆) δ 8.34 (s, 1H), 8.25 (s, 1H), 7.96 (dd, J = 8.6, 1H), 7.75 (t, JH-F = 72.9 Hz, 1H)), 7.69 (s, 1H), 7.61 (d, J = 8.2 Hz, 1H), 7.57 (d, J = 9.2 Hz, 1H), 7.27 (d, J = 9.2 Hz, 1H), 7.18 (d, J = 8.6 Hz, 1H), 6.44 (d, J = 8.2 Hz, 1H), 4.92 (s, 2H), 4.15 (s, 3H), 3.87 (q, J = 7.0 Hz, 2H), 1.08 (t, J = 7.0 Hz, 3H). |

### Synthesis of 4-(3-(4-methoxyphenyl)-2-oxo-7-((2,2,2-trifluoroethyl)amino)-3,4-dihydropyrimido[4,5-d]pyrimidin-1(2H)-yl)benzamide (Example 276)

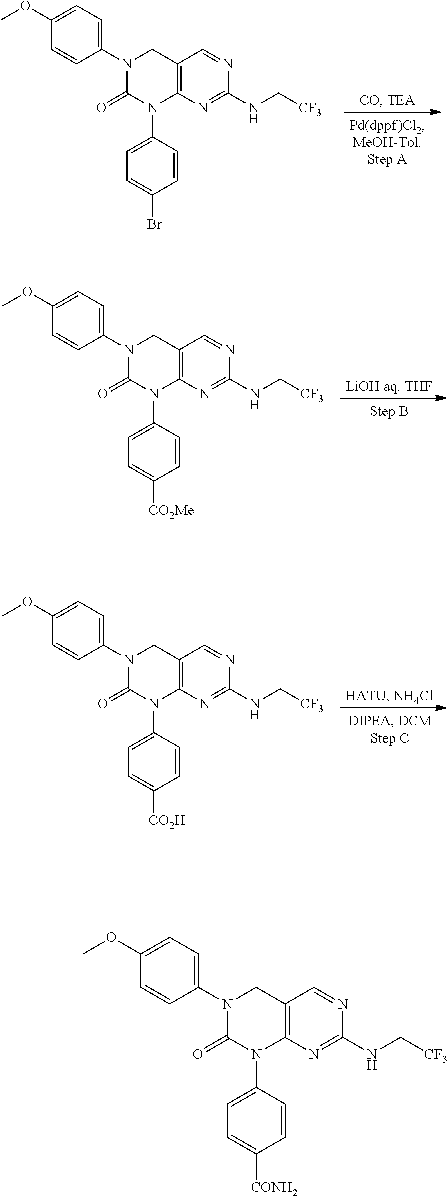 [SMILES: COc1ccc(N2Cc3cnc(NCC(F)(F)F)nc3N(c3ccc(Br)cc3)C2=O)cc1 ; COC(=O)c1ccc(N2C(=O)N(c3ccc(OC)cc3)Cc3cnc(NCC(F)(F)F)nc32)cc1 ; COc1ccc(N2Cc3cnc(NCC(F)(F)F)nc3N(c3ccc(C(=O)O)cc3)C2=O)cc1 ; COc1ccc(N2Cc3cnc(NCC(F)(F)F)nc3N(c3ccc(C(N)=O)cc3)C2=O)cc1]

### Step A: methyl 4-(3-(4-methoxyphenyl)-2-oxo-7-((2,2,2-trifluoroethyl)amino)-3,4-dihydropyrimido[4,5-d]pyrimidin-1(2H)-yl)benzoate

A mixture of 1-(4-bromophenyl)-3-(4-methoxyphenyl)-7-(2,2,2-trifluoroethylamino)-3,4-dihydropyrimido[4,5-d]pyrimidin-2(1H)-one (synthesized from N-((4-chloro-2-(methylthio)pyrimidin-5-yl)methyl)-4-methoxyaniline & 1-bromo-4-isocyanatobenzene via General Procedure VI (Step B-E)) (355 mg, 0.7 mmol, 1.0 eq.), TEA (212 mg, 2.1 mmol, 3.0 eq.) and Pd(dppf)Cl₂ (51 mg, 0.07 mmol, 0.1 eq.) in toluene/MeOH (10 mL, 10/1) was stirred at 100° C. for 14 hrs under CO atmosphere. Then the reaction mixture was poured into H₂O (10 mL) and extracted with EtOAc (10 mL×3). The combined organic layers were washed with brine (20 mL), dried over Na₂SO₄ and concentrated under reduced pressure, the residue was purified by flash column chromatography on silica gel to give methyl 4-(3-(4-methoxyphenyl)-2-oxo-7-((2,2,2-trifluoroethyl)amino)-3,4-dihydropyrimido[4,5-d]pyrimidin-1(2H)-yl)benzoate (300 mg, 88% yield) as a yellow solid. LC-MS (ESI): m/z 488 [M+H]⁺.

### Step B: 4-(3-(4-methoxyphenyl)-2-oxo-7-((2,2,2-trifluoroethyl)amino)-3,4-dihydropyrimido[4,5-d]pyrimidin-1(2H)-yl)benzoic acid

A solution of methyl 4-(3-(4-methoxyphenyl)-2-oxo-7-((2,2,2-trifluoroethyl)amino)-3,4-dihydropyrimido[4,5-d]pyrimidin-1(2H)-yl)benzoate (292 mg, 0.6 mmol, 1.0 eq.) and LiOH (aq.) (1N, 6 mL, 6 mmol, 10.0 eq.) in THF (6 mL) was stirred at room temperature overnight. Adding diluted HCl (1N, aq.) to adjust pH=6, the resulting mixture was extracted with EtOAc (10 mL×3). The combined organic layers were washed with brine (30 mL), dried over Na₂SO₄ and concentrated under reduced pressure, the residue was purified by flash column chromatography on silica gel to give 4-(3-(4-methoxyphenyl)-2-oxo-7-((2,2,2-trifluoroethyl)amino)-3,4-dihydropyrimido[4,5-d]pyrimidin-1(2H)-yl)benzoic acid (244 mg, 86% yield) as a white solid. LC-MS (ESI): m/z 474 [M+H]⁺.

### Step C: 4-(3-(4-methoxyphenyl)-2-oxo-7-((2,2,2-trifluoroethyl)amino)-3,4-dihydropyrimido[4,5-d]pyrimidin-1(2H)-yl)benzamide

A mixture of 4-(3-(4-methoxyphenyl)-2-oxo-7-((2,2,2-trifluoroethyl)amino)-3,4-dihydropyrimido[4,5-d]pyrimidin-1(2H)-yl)benzoic acid (47 mg, 0.1 mmol, 1.0 eq.), NH₄Cl (32 mg, 0.6 mmol, 6.0 eq.), DIPEA (77 mg, 0.6 mmol, 6.0 eq.) and HATU (76 mg, 0.2 mmol, 2.0 eq.) in DCM (3 mL) was stirred at room temperature for 1 hr. Then the reaction mixture was poured into H₂O (10 mL) and extracted with DCM (10 mL×3). The combined organic layers were washed with brine (20 mL), dried over Na₂SO₄ and concentrated under reduced pressure, the residue was purified by RP-prep-HPLC to afford 4-(3-(4-methoxyphenyl)-2-oxo-7-((2,2,2-trifluoroethyl)amino)-3,4-dihydropyrimido[4,5-d]pyrimidin-1(2H)-yl)benzamide (Example 276).

¹H NMR (400 MHz, DMSO-d₆) δ (ppm): 8.19 (s, 1H), 8.09 (s, 1H), 7.99 (d, J=8.4 Hz, 2H), 7.56 (br s, 1H), 7.45-7.40 (m, 3H), 7.40 (d, J=8.8 Hz, 2H), 7.03 (d, J=8.8 Hz, 2H), 4.83 (s, 2H), 4.00-3.50 (m, 2H), 3.83 (s, 3H).

LC-MS (ESI): m/z 473 [M+H]⁺.

### Synthesis of 4-(3-(4-methoxyphenyl)-2-oxo-7-((2,2,2-trifluoroethyl)amino)-3,4-dihydropyrimido[4,5-d]pyrimidin-1(2H)-yl)-N-methylbenzamide (Example 277)

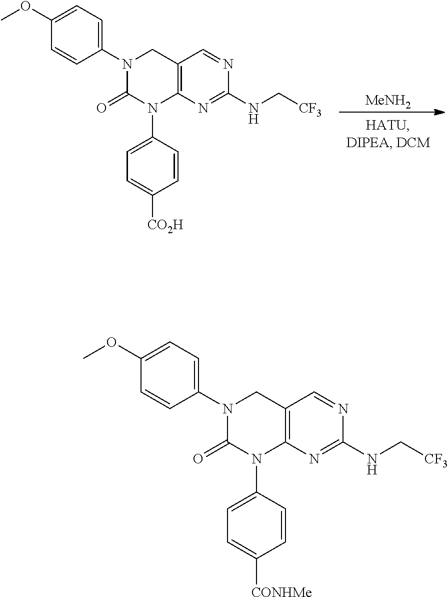

The procedure to obtain Example 276 (Step C) was followed. Thus, 4-(3-(4-methoxyphenyl)-2-oxo-7-((2,2,2-trifluoroethyl)amino)-3,4-dihydropyrimido[4,5-d]pyrimidin-1(2H)-yl)-N-methylbenzamide (Example 277) was synthesized from 4-(3-(4-methoxyphenyl)-2-oxo-7-((2,2,2-trifluoroethyl)amino)-3,4-dihydropyrimido[4,5-d]pyrimidin-1(2H)-yl)benzoic acid (Example 276, Step B) and methylamine hydrochloride.

¹H NMR (400 MHz, DMSO-d₆) δ (ppm): 8.56 (q, J=4.0 Hz, 1H), 8.19 (s, 1H), 7.95 (d, J=8.4 Hz, 2H), 7.55 (br s, 1H), 7.44 (d, J=8.4 Hz, 2H), 7.40 (d, J=8.8 Hz, 2H), 7.03 (d, J=9.2 Hz, 2H), 4.77 (s, 2H), 4.00-3.50 (m, 2H), 3.76 (s, 3H), 2.80 (d, J=4.4 Hz, 3H).

LC-MS (ESI): m/z 487 [M+H]⁺.

### Synthesis of 1-(4-(1-hydroxycyclopropyl)phenyl)-3-(4-methoxyphenyl)-7-((2,2,2-trifluoroethyl)amino)-3,4-dihydropyrimido[4,5-d]pyrimidin-2(1H)-one (Example 278)

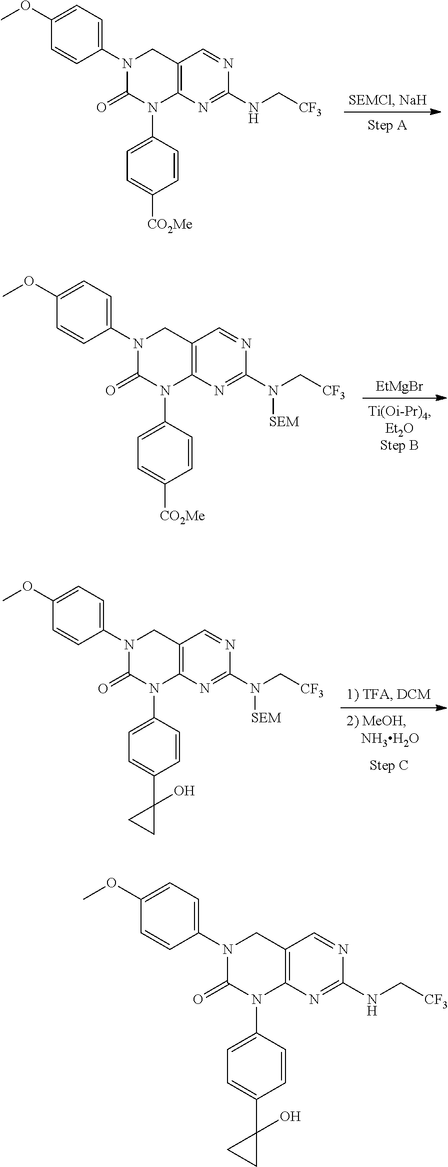

### Step A: methyl 4-(3-(4-methoxyphenyl)-2-oxo-7-((2,2,2-trifluoroethyl)((2-(trimethylsilyl)ethoxy)methyl)amino)-3,4-dihydropyrimido[4,5-d]pyrimidin-1(2H)-yl)benzoate

To a solution of methyl 4-(3-(4-methoxyphenyl)-2-oxo-7-((2,2,2-trifluoroethyl)amino)-3,4-dihydropyrimido[4,5-d]pyrimidin-1(2H)-yl)benzoate (Example 276, Step A) (500 mg, 1.03 mmol, 1.0 eq.) in DMF (5 mL) was added NaH (60% in mineral oil, 82 mg, 2.06 mmol, 2.0 eq.) at 0° C. The mixture was stirred at 0° C. for 0.5 hr. Then SEMCl (256 mg, 1.53 mmol, 1.49 eq.) was added. The reaction mixture was stirred at room temperature for 1 hr and quenched with NH₄Cl (sat. aq.) (10 mL) at 0° C. Then the reaction mixture was extracted with EtOAc (10 mL×3). The combined organic layers were washed with brine (20 mL), dried over Na₂SO₄ and concentrated under reduced pressure, the residue was purified by flash column chromatography on silica gel to give methyl 4-(3-(4-methoxyphenyl)-2-oxo-7-((2,2,2-trifluoroethyl)((2-(trimethylsilyl)ethoxy)methyl)amino)-3,4-dihydropyrimido[4,5-d]pyrimidin-1(2H)-yl)benzoate (550 mg, 87% yield) as a white solid.

LC-MS (ESI): m/z 618 [M+H]⁺.

### Step B: 1-(4-(1-hydroxycyclopropyl)phenyl)-3-(4-methoxyphenyl)-7-((2,2,2-trifluoroethyl)((2-(trimethylsilyl)ethoxy)methyl)amino)-3,4-dihydropyrimido[4,5-d]pyrimidin-2(1H)-one

To a solution of methyl 4-(3-(4-methoxyphenyl)-2-oxo-7-((2,2,2-trifluoroethyl)((2-(trimethylsilyl)ethoxy)methyl)amino)-3,4-dihydropyrimido[4,5-d]pyrimidin-1(2H)-yl)benzoate (240 mg, 0.39 mmol, 1.0 eq.) in diethyl ether (3 mL) was added titanium(IV) isopropoxide (222 mg, 0.78 mmol, 2.0 eq.) at −78° C. drop-wisely. After 10 min, EtMgBr (1M in THF, 2.3 mL, 2.33 mmol, 6.0 eq.) was added drop-wisely. Then the resulting mixture was allowed to warm up to room temperature and stirred for additional 4 hrs. The resulting mixture was quenched with NH₄Cl (sat. aq.) (10 mL) and extracted with EtOAc (10 mL×3). The combined organic layers were washed with brine (20 mL), dried over Na₂SO₄ and concentrated under reduced pressure, the residue was purified by flash column chromatography on silica gel to give 1-(4-(1-hydroxycyclopropyl)phenyl)-3-(4-methoxyphenyl)-7-((2,2,2-trifluoroethyl)((2-(trimethylsilyl)ethoxy)methyl)amino)-3,4-dihydropyrimido[4,5-d]pyrimidin-2(1H)-one (74 mg, 31% yield). LC-MS ESI m/z=616 [M+H]⁺.

### Step C: 1-(4-(1-hydroxycyclopropyl)phenyl)-3-(4-methoxyphenyl)-7-((2,2,2-trifluoroethyl)amino)-3,4-dihydropyrimido[4,5-d]pyrimidin-2(1H)-one (Example 278) was synthesized from 1-(4-(1-hydroxycyclopropyl)phenyl)-3-(4-methoxyphenyl)-7-((2,2,2-trifluoroethyl)((2-(trimethylsilyl)ethoxy)methyl)amino)-3,4-dihydropyrimido[4,5-d]pyrimidin-2(1H)-one via General Procedure IV (Method A, Step F)

¹H NMR (400 MHz, CDCl₃) δ (ppm): 7.92 (s, 1H), 7.31 (d, J=8.4 Hz, 2H), 7.22 (d, J=9.2 Hz, 2H), 7.19-7.16 (m, 2H), 6.85 (d, J=9.2 Hz, 2H), 5.10 (br s, 1H), 4.69 (s, 2H), 3.95-3.59 (m, 2H), 3.74 (s, 3H), 1.27-1.21 (m, 2H), 1.05-0.99 (m, 2H).

LC-MS (ESI): m/z 486 [M+H]⁺.

### Synthesis of 3-(4-methoxyphenyl)-1-(4-(2-oxo-1,2-dihydropyridin-3-yl)phenyl)-7-((2,2,2-trifluoroethyl)amino)-3,4-dihydropyrimido[4,5-d]pyrimidin-2(1H)-one (Example 279)

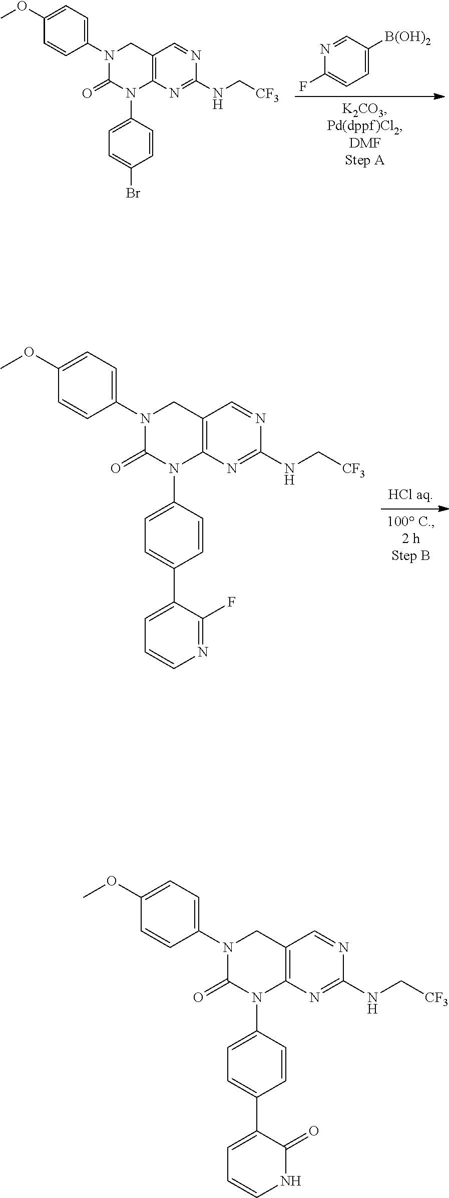

### Step A: 1-(4-(2-fluoropyridin-3-yl)phenyl)-3-(4-methoxyphenyl)-7-((2,2,2-trifluoroethyl)amino)-3,4-dihydropyrimido[4,5-d]pyrimidin-2(1H)-one

A mixture of 1-(4-bromophenyl)-3-(4-methoxyphenyl)-7-((2,2,2-trifluoroethyl)amino)-3,4-dihydropyrimido[4,5-d]pyrimidin-2(1H)-one (synthesized from N-((4-chloro-2-(methylthio)pyrimidin-5-yl)methyl)-4-methoxy aniline and 1-bromo-4-isocyanatobenzene via General Procedure VI (Step B-E)) (101 mg, 0.2 mmol, 1.0 eq.), (6-fluoropyridin-3-yl)boronic acid (56 mg, 0.4 mmol, 2.0 eq.), Pd(dppf)Cl₂ (15 mg, 0.02 mmol, 0.1 eq.) and K₂CO₃ (83 mg, 0.6 mmol, 3.0 eq.) in DMF (5 mL) was stirred at 100° C. for 14 h under N₂ atmosphere. The reaction mixture was cooled to room temperature, diluted with water (10 mL), extracted with EtOAc (10 mL×3). The combined organic layers were washed with brine (2 mL), dried over Na₂SO₄ and concentrated under reduced pressure, the residue was purified by flash column chromatography on silica gel to give 1-(4-(2-fluoropyridin-3-yl)phenyl)-3-(4-methoxyphenyl)-7-((2,2,2-trifluoroethyl)amino)-3,4-dihydropyrimido[4,5-d]pyrimidin-2(1H)-one (100 mg, 95% yield) as a white solid. LC-MS (ESI): m/z 525 [M+H]⁺.

### Step B: 3-(4-methoxyphenyl)-1-(4-(2-oxo-1,2-dihydropyridin-3-yl)phenyl)-7-((2,2,2-trifluoroethyl)amino)-3,4-dihydropyrimido[4,5-d]pyrimidin-2(1H)-one

A solution of 1-(4-(2-fluoropyridin-3-yl)phenyl)-3-(4-methoxyphenyl)-7-((2,2,2-trifluoroethyl)amino)-3,4-dihydropyrimido[4,5-d]pyrimidin-2(1H)-one (53 mg, 0.1 mmol, 1.0 eq.) in HCl (1N, aq.) (3 mL) was stirred at 100° C. for 2 hrs. The reaction mixture was concentrated under reduced pressure and directly purified by RP-prep-HPLC to afford 3-(4-methoxyphenyl)-1-(4-(2-oxo-1,2-dihydropyridin-3-yl)phenyl)-7-((2,2,2-trifluoroethyl)amino)-3,4-dihydropyrimido[4,5-d]pyrimidin-2(1H)-one (Example 279).

¹H NMR (400 MHz, DMSO-d₆) δ (ppm): 11.78 (br s, 1H), 8.13 (s, 1H), 7.80 (d, J=8.4 Hz, 2H), 7.70 (dd, J=10.0 Hz, 2.0 Hz, 1H), 7.53 (br s, 1H), 7.41 (dd, J=10.0 Hz, 2.0 Hz, 1H), 7.35 (d, J=8.8 Hz, 2H), 7.30 (d, J=8.4 Hz, 2H), 6.97 (d, J=9.2 Hz, 2H), 6.32 (t, J=6.4 Hz, 1H), 4.77 (s, 2H), 4.07-3.81 (m, 2H), 3.77 (s, 3H).

LC-MS (ESI): m/z 523 [M+H]⁺.

### Synthesis of 1-(4-(1H-pyrrol-3-yl)phenyl)-3-(4-methoxyphenyl)-7-((2,2,2-trifluoroethyl)amino)-3,4-dihydropyrimido[4,5-d]pyrimidin-2(1H)-one (Example 280)

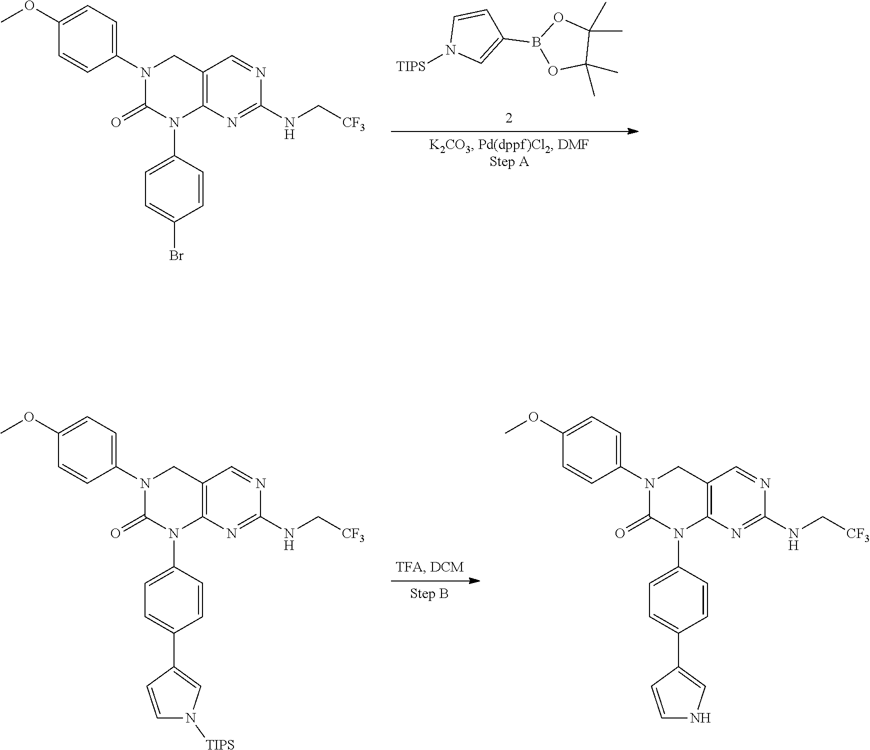

Step A: 3-(4-methoxyphenyl)-7-((2,2,2-trifluoroethyl)amino)-1-(4-(1-(triisopropylsilyl)-1H-pyrrol-3-yl)phenyl)-3,4-dihydropyrimido[4,5-d]pyrimidin-2(1H)-one was synthesized via similar procedure via Example 279 (Step A) from 1-(4-bromophenyl)-3-(4-methoxyphenyl)-7-((2,2,2-trifluoroethyl)amino)-3,4-dihydropyrimido[4,5-d]pyrimidin-2(1H)-one and 3-(4,4,5,5-tetramethyl-1,3,2-dioxaborolan-2-yl)-1-(triisopropylsilyl)-1H-pyrrole (Ref: Eur. J Med. Chem., 2015, 103, 105-122) as a white solid. LC-MS (ESI): m/z 651 [M+H]⁺.

### Step B: 1-(4-(1H-pyrrol-3-yl)phenyl)-3-(4-methoxyphenyl)-7-((2,2,2-trifluoroethyl)amino)-3,4-dihydropyrimido[4,5-d]pyrimidin-2(1H)-one

A solution of 3-(4-methoxyphenyl)-7-((2,2,2-trifluoroethyl)amino)-1-(4-(1-(triisopropylsilyl)-1H-pyrrol-3-yl)phenyl)-3,4-dihydropyrimido[4,5-d]pyrimidin-2(1H)-one (65 mg, 0.1 mmol) in TFA/DCM mixture (3 mL, 1/5, v/v) was stirred at room temperature for 14 hrs. Then the reaction mixture was concentrated under reduced pressure and purified by RP-prep-HPLC to afford 1-(4-(1H-pyrrol-3-yl)phenyl)-3-(4-methoxyphenyl)-7-((2,2,2-trifluoroethyl)amino)-3,4-dihydropyrimido[4,5-d]pyrimidin-2(1H)-one (Example 280).

¹H NMR (400 MHz, DMSO-d₆) δ (ppm): 10.95 (s, 1H), 8.11 (s, 1H), 7.57 (d, J=8.4 Hz, 2H), 7.48 (br s, 1H), 7.34 (d, J=8.8 Hz, 2H), 7.25 (d, J=2.0 Hz, 1H), 7.18 (d, J=8.4 Hz, 2H), 6.97 (d, J=8.8 Hz, 2H), 6.81 (dd, J=4.4 Hz, 2.4 Hz, 1H), 6.46 (dd, J=4.0 Hz, 2.4 Hz, 1H), 4.76 (s, 2H), 4.15-3.68 (m, 2H), 3.77 (s, 3H).

LC-MS (ESI): m/z 495 [M+H]⁺.

### Synthesis of 1-(4-(2-hydroxycyclopropyl)phenyl)-3-(4-methoxyphenyl)-7-((2,2,2-trifluoroethyl)amino)-3,4-dihydropyrimido[4,5-d]pyrimidin-2(1H)-one (Example 281)

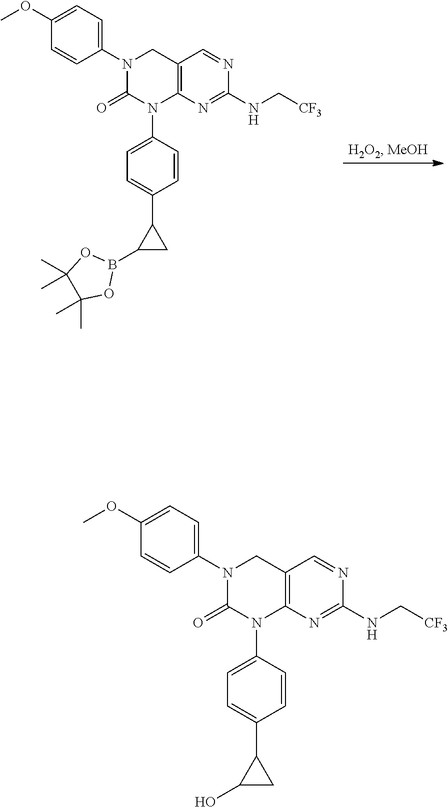

A solution of 3-(4-methoxyphenyl)-1-(4-(2-(4,4,5,5-tetramethyl-1,3,2-dioxaborolan-2-yl)cyclopropyl)phenyl)-7-((2,2,2-trifluoroethyl)amino)-3,4-dihydropyrimido[4,5-d]pyrimidin-2(1H)-one (synthesized from 3-(4-methoxyphenyl)-7-((2,2,2-trifluoroethyl)amino)-3,4-dihydropyrimido[4,5-d]pyrimidin-2(1H)-one and 2-(2-(4-bromophenyl)cyclopropyl)-4,4,5,5-tetramethyl-1,3,2-dioxaborolane (Ref: Organic and Biomolecular Chemistry, 2016, 14, 6591-6595) via General Procedure IV (Method B, Step C)) (100 mg, 0.17 mmol, 1.0 eq.) and H₂O₂ (30% in H₂O, 0.34 mL, 3.4 mmol, 20 eq.) in MeOH (3 mL) was stirred at 0° C. for 2 hrs. Then the reaction mixture was diluted with water (10 mL) and extracted with EtOAc (10 mL×3). The combined organic layers were washed with brine (20 mL), dried over Na₂SO₄ and concentrated under reduced pressure, the residue was purified by RP-prep-HPLC to afford 1-(4-(2-hydroxycyclopropyl)phenyl)-3-(4-methoxyphenyl)-7-((2,2,2-trifluoroethyl)amino)-3,4-dihydropyrimido[4,5-d]pyrimidin-2(1H)-one (Example 281).

¹H NMR (400 MHz, DMSO-d₆) δ (ppm): 8.11 (s, 1H), 7.33 (d, J=8.8 Hz, 2H), 7.13 (d, J=8.4 Hz, 2H), 7.05 (d, J=8.4 Hz, 2H), 6.96 (d, J=8.8 Hz, 2H), 4.76 (s, 2H), 3.95-3.83 (m, 2H), 3.77 (s, 3H), 3.39-3.33 (m, 1H), 2.00-1.93 (m, 1H), 1.15-1.09 (m, 1H), 0.98-0.93 (m, 1H).

LC-MS (ESI): m/z 486 [M+H]⁺.

### Synthesis of 7-ethoxy-1-(6-(fluoromethyl)pyridin-3-yl)-3-(2-methyl-2H-indazol-5-yl)-3,4-dihydropyrido[2,3-d]pyrimidin-2(1H)-one (Example 282)

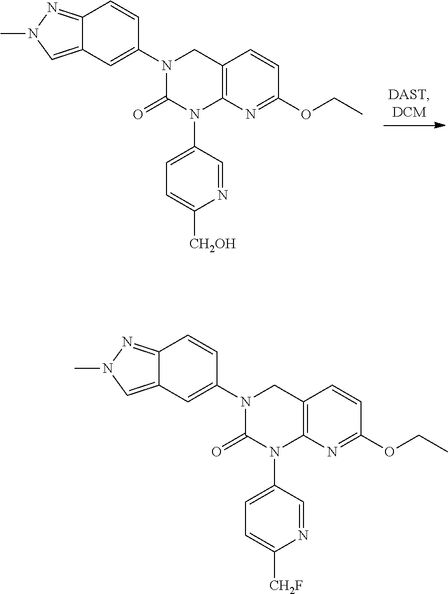

To a solution of 7-ethoxy-1-(6-(hydroxymethyl)pyridin-3-yl)-3-(2-methyl-2H-indazol-5-yl)-3,4-dihydropyrido[2,3-d]pyrimidin-2(1H)-one (synthesized from 7-ethoxy-3-(2-methyl-2H-indazol-5-yl)-3,4-dihydropyrido[2,3-d]pyrimidin-2(1H)-one and (5-bromopyridin-2-yl)methanol via General Procedure II (Method A, Step D)) (112 mg, 0.26 mmol, 1.0 eq.) in DCM (10 mL) was added DAST (84 mg, 0.52 mmol, 2.0 eq.) at 0° C. Then the reaction mixture was stirred for 30 min. The resulting mixture was quenched with ice water (10 mL), and extracted with EtOAc (10 mL×3). The combined organic layers were washed with brine (20 mL), dried over Na₂SO₄ and concentrated under reduced pressure, the residue was purified by RP-prep-HPLC to afford 7-ethoxy-1-(6-(fluoromethyl)pyridin-3-yl)-3-(2-methyl-2H-indazol-5-yl)-3,4-dihydropyrido[2,3-d]pyrimidin-2(1H)-one (Example 282).

¹H NMR (400 MHz, DMSO-d₆) δ (ppm): 8.57 (d, J=2.4 Hz, 1H), 8.36 (s, 1H), 7.92 (dd, J=8.0 Hz, 2.4 Hz, 1H), 7.72 (dd, J=2.0 Hz, 0.8 Hz, 1H), 7.66-7.55 (m, 3H), 7.30 (dd, J=9.2 Hz, 2.0 Hz, 1H), 6.46 (d, J=8.0 Hz, 1H), 5.54 (d, J_{HF}=48.0 Hz, 2H), 4.94 (s, 2H), 4.17 (s, 3H), 3.86 (q, J=7.2 Hz, 2H), 1.07 (t, J=7.2 Hz, 3H).

LC-MS (ESI): m/z 433 [M+H]⁺.

### Synthesis of 1-(benzo[d]oxazol-5-yl)-7-(2,2-difluoroethoxy)-3-(2-methyl-2H-indazol-5-yl)-3,4-dihydropyrido[2,3-d]pyrimidin-2(1H)-one (Example 283)

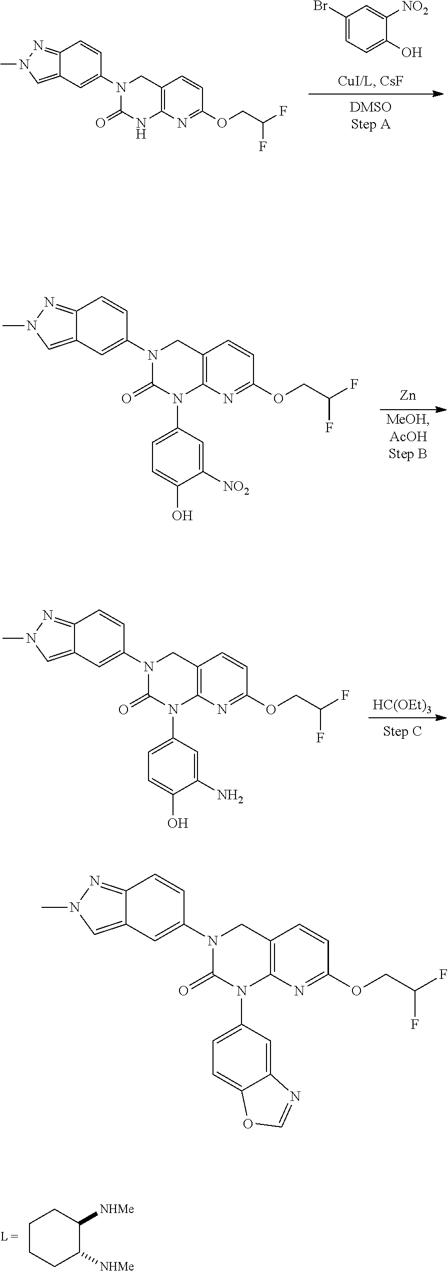

### Step A: 7-(2,2-difluoroethoxy)-1-(4-hydroxy-3-nitrophenyl)-3-(2-methyl-2H-indazol-5-yl)-3,4-dihydropyrido[2,3-d]pyrimidin-2(1H)-one

To a solution of 7-(2,2-difluoroethoxy)-3-(2-methyl-2H-indazol-5-yl)-3,4-dihydropyrido[2,3-d]pyrimidin-2(1H)-one (500 mg, 1.39 mmol, 1.0 eq.) (obtained via General Procedure II, Method A, Steps A-C) in DMSO (2 mL) was added CsF (630 mg, 4.08 mmol, 3.0 eq.), N¹,N²-dimethylcyclohexane-1,2-diamine (395 mg, 2.78 mmol, 2.0 eq.) and CuI (264 mg, 1.39 mmol, 1.0 eq.), the reaction mixture was stirred at 100° C. under N₂ atmosphere for 16 hrs. The progress of the reaction was monitored by LC-MS, after completion, the reaction mixture diluted with water (20 mL) and extracted with EtOAc (30 mL×3), the combined organic layers were washed with brine (30 mL), dried over Na₂SO₄ and concentrated under reduced pressure, the residue was purified by flash column chromatography on silica gel to afford 7-(2,2-difluoroethoxy)-1-(4-hydroxy-3-nitrophenyl)-3-(2-methyl-2H-indazol-5-yl)-3,4-dihydropyrido[2,3-d]pyrimidin-2(1H)-one (432 mg, 56% yield) as a yellow solid. LC-MS (ESI): m/z 497 [M+H]⁺

### Step B: 1-(3-amino-4-hydroxyphenyl)-7-(2,2-difluoroethoxy)-3-(2-methyl-2H-indazol-5-yl)-3,4-dihydropyrido[2,3-d]pyrimidin-2(1H)-one

To a solution of 7-(2,2-difluoroethoxy)-1-(4-hydroxy-3-nitrophenyl)-3-(2-methyl-2H-indazol-5-yl)-1H,2H,3H,4H-pyrido[2,3-d]pyrimidin-2-one (200 mg, 0.4 mmol, 1.0 eq.) in MeOH (2 mL) was added acetic acid (0.5 mL) and zinc (79 mg, 1.20 mmol, 3.0 eq.), the reaction mixture was stirred at room temperature for 2 hrs. The reaction mixture was diluted with water (10 mL) and extracted with EtOAc (10 mL×3), the combined organic layers were washed with brine (20 mL), dried over Na₂SO₄ and concentrated under reduced pressure, the residue was purified by flash column chromatography on silica gel to afford 1-(3-amino-4-hydroxyphenyl)-7-(2,2-difluoroethoxy)-3-(2-methyl-2H-indazol-5-yl)-3,4-dihydropyrido[2,3-d]pyrimidin-2(1H)-one (138 mg, 73% yield) as a yellow solid. LC-MS (ESI): m/z 467 [M+H]⁺.

### Step C: 1-(benzo[d]oxazol-5-yl)-7-(2,2-difluoroethoxy)-3-(2-methyl-2H-indazol-5-yl)-3,4-dihydropyrido[2,3-d]pyrimidin-2(1H)-one

A suspension of 1-(3-amino-4-hydroxyphenyl)-7-(2,2-difluoroethoxy)-3-(2-methyl-2H-indazol-5-yl)-1H,2H,3H,4H-pyrido[2,3-d]pyrimidin-2-one (118 mg, 0.25 mmol, 1.0 eq.) in HC(OEt)₃ (4 mL) was stirred at 120° C. for 8 hrs. The progress of the reaction was monitored by LC-MS, after completion, excess of HC(OEt)₃ was removed under reduced pressure, the residue was diluted with water (20 mL) and extracted with EtOAc (20 mL×3). the combined organic layers were washed with brine (20 mL), dried over Na₂SO₄ and concentrated under reduced pressure, the residue was purified by RP-prep-HPLC to afford 1-(benzo[d]oxazol-5-yl)-7-(2,2-difluoroethoxy)-3-(2-methyl-2H-indazol-5-yl)-3,4-dihydropyrido[2,3-d]pyrimidin-2(1H)-one (Example 283). LC-MS (ESI): m/z 477 [M+H]+

¹H NMR (400 MHz, DMSO-d₆) δ (ppm): 8.79 (s, 1H), 8.36 (s, 1H), 7.86-7.81 (m, 2H), 7.72 (d, J=2.0 Hz, 1H), 7.69 (d, J=8.0 Hz, 1H), 7.58 (d, J=9.2 Hz, 1H), 7.45 (dd, J=8.8 Hz, 1.6 Hz, 1H), 7.30 (dd, J=9.2 Hz, 1.6 Hz, 1H), 6.55 (d, J=8.0 Hz, 1H), 5.94 (tt, J_{HF}=55.2 Hz, J=4.0 Hz, 1H), 4.98 (s, 2H), 4.17 (s, 3H), 3.97 (td, J_{HF}=14.4 Hz, J=3.6 Hz, 2H).

### Synthesis of 6-chloro-7-(2,2-difluoroethoxy)-1-(4-(difluoromethoxy)phenyl)-3-(2-methyl-2H-indazol-5-yl)-3,4-dihydroquinazolin-2(1H)-one (Example 284)

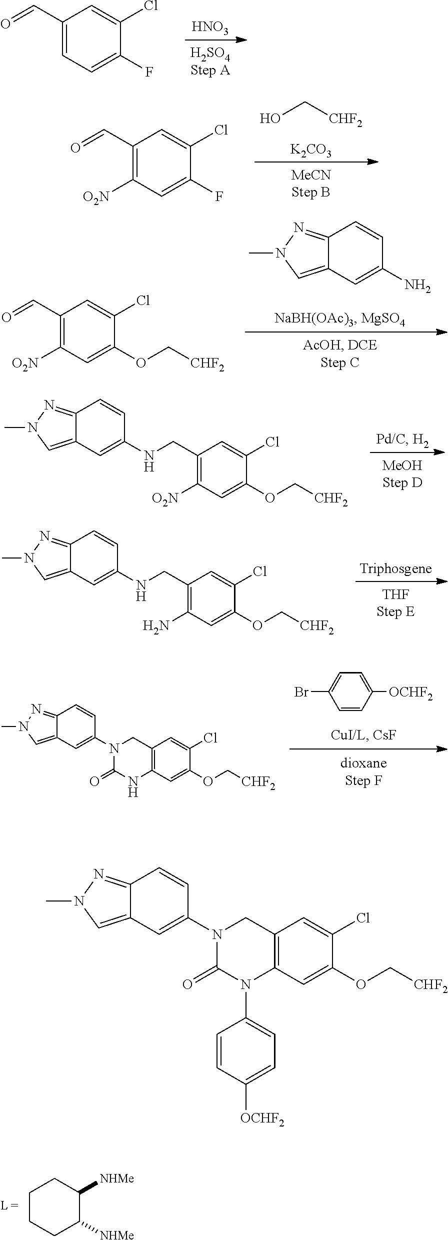

### Step A: 5-chloro-4-fluoro-2-nitrobenzaldehyde

To a solution of 3-chloro-4-fluorobenzaldehyde (1 g, 6.30 mmol, 1.0 eq.) in H₂SO₄ (4 mL) was added HNO₃ (1 mL) carefully at 0° C., the reaction mixture was allowed to warm to room temperature and stirred for additional 2 hrs. The reaction mixture was poured onto ice water (10 mL) and extracted with EtOAc (40 mL×3), the combined organic layers were washed with brine (30 ml), dried over with Na₂SO₄ and concentrated under reduced pressure, the residue was purified by flash column chromatography on silica gel to afford 5-chloro-4-fluoro-2-nitrobenzaldehyde (960 mg, 75% yield) as a white solid.

### Step B: 5-chloro-4-(2,2-difluoroethoxy)-2-nitrobenzaldehyde

To a solution of 5-chloro-4-fluoro-2-nitrobenzaldehyde (50 mg, 0.24 mmol, 1.0 eq.) in acetonitrile (4 mL) was added K₂CO₃ (68 mg, 0.49 mmol, 2.0 eq.) and 2,2-difluoroethan-1-ol (24 mg, 0.29 mmol, 1.2 eq.), the reaction mixture was stirred at 80° C. for 15 hrs. The reaction mixture was diluted with H₂O (10 ml), extracted with EtOAc (10 mL×3), the combined organic layers were washed with brine (10 ml), dried over with Na₂SO₄ and concentrated under reduced pressure, the residue was purified by flash column chromatography on silica gel to afford 5-chloro-4-(2,2-difluoroethoxy)-2-nitrobenzaldehyde (25 mg, 38% yield). LC-MS (ESI): m/z 266 [M+H]⁺.

### Step C: N-(5-chloro-4-(2,2-difluoroethoxy)-2-nitrobenzyl)-2-methyl-2H-indazol-5-amine

To a mixture of 5-chloro-4-(2,2-difluoroethoxy)-2-nitrobenzaldehyde (400 mg, 1.50 mmol, 1.0 eq.) and 2-methyl-2H-indazol-5-amine (244 mg, 1.65 mmol, 1.1 eq.) in DCE (4 mL) was added MgSO₄ (1807 mg, 15.06 mmol, 10.0 eq.) and HOAc (361 mg, 6.02 mmol, 4.0 eq.), the reaction mixture was stirred at 25° C. for 16 hrs. NaBH(OAc)₃ (477 mg, 2.25 mmol, 3.0 eq.) was added in several portions at 0° C., after which the reaction mixture was allowed to warm up to room temperature and stirred for additional 4 hrs. The progress of the reaction was monitored by LC-MS, after completion, the reaction was quenched with NaHCO₃ (sat. aq.) (20 mL), extracted with EtOAc (20 mL×3), the combined organic layers were washed with brine (20 mL), dried over with Na₂SO₄ and concentrated under reduced pressure, the residue was purified by flash column chromatography on silica gel to afford N-(5-chloro-4-(2,2-difluoroethoxy)-2-nitrobenzyl)-2-methyl-2H-indazol-5-amine (472 mg, 79% yield) as a pale yellow solid. LC-MS (ESI): m/z 397 [M+H]⁺.

### Step D: N-(5-chloro-4-(2,2-difluoroethoxy)-2-nitrobenzyl)-2-methyl-2H-indazol-5-amine

To a solution of N-(5-chloro-4-(2,2-difluoroethoxy)-2-nitrobenzyl)-2-methyl-2H-indazol-5-amine (2.0 g, 5.04 mmol, 1.0 eq) in MeOH (10 mL) was added Pd/C (200 mg, 10% wt, wet), the reaction mixture was stirred under H₂ balloon (1 atm) at 25° C. for 3 hrs. The progress of the reaction was monitored by TLC, after completion, the catalyst was removed by filtering through a short pad of Celite®, the filtrate was concentrated under reduced pressure and the residue was purified by flash column chromatography on silica gel to afford give N-(5-chloro-4-(2,2-difluoroethoxy)-2-nitrobenzyl)-2-methyl-2H-indazol-5-amine (800 mg, 43%) as an off-white solid. LC-MS (ESI): m/z 367 [M+H]⁺.

### Step E: 6-chloro-7-(2,2-difluoroethoxy)-3-(2-methyl-2H-indazol-5-yl)-3,4-dihydroquinazolin-2(1H)-one

To a solution of N-(5-chloro-4-(2,2-difluoroethoxy)-2-nitrobenzyl)-2-methyl-2H-indazol-5-amine (300 mg, 0.81 mmol, 1.0 eq.) in THF (5 mL) was added triphosgene (97 mg, 0.32 mmol, 0.4 eq.) at 0° C., was allowed to warm to room temperature and stirred for 16 hrs. The reaction was quenched with NaHCO₃ (sat. aq.) (20 mL), extracted with EtOAc (40 mL×3), the combined organic layers were washed with brine (20 mL), dried over with Na₂SO₄ and concentrated under reduced pressure, the residue was purified by flash column chromatography on silica gel to afford 6-chloro-7-(2,2-difluoroethoxy)-3-(2-methyl-2H-indazol-5-yl)-3,4-dihydroquinazolin-2(1H)-one (130 mg, 40% yield) as a white solid. LC-MS (ESI): m/z 367 [M+H]⁺.

### Step F: 6-chloro-7-(2,2-difluoroethoxy)-1-(4-(difluoromethoxy)phenyl)-3-(2-methyl-2H-indazol-5-yl)-3,4-dihydroquinazolin-2(1H)-one

To a solution of 6-chloro-7-(2,2-difluoroethoxy)-3-(2-methyl-2H-indazol-5-yl)-1,2,3,4-tetrahydroquinazolin-2-one (87 mg, 0.22 mmol, 1.0 eq.) in dioxane (3 mL) was added 1-bromo-4-(difluoromethoxy)benzene (74 mg, 0.33 mmol, 1.5 eq.), CsF (101 mg, 0.66 mmol, 3.0 eq.), N¹,N²-dimethylcyclohexane-1,2-diamine (63 mg, 0.44 mmol, 2.0 eq.) and CuI (42 mg, 0.22 mmol, 1.0 eq.), the reaction mixture was stirred at 90° C. under N₂ atmosphere for 16 hrs. The reaction mixture was diluted with H₂O (20 mL), extracted with EtOAc (20 mL×3), dried over Na₂SO₄ and concentrated under reduced pressure, the residue was purified by RP-prep-HPLC to afford 6-chloro-7-(2,2-difluoroethoxy)-1-(4-(difluoromethoxy)phenyl)-3-(2-methyl-2H-indazol-5-yl)-3,4-dihydroquinazolin-2(1H)-one (Example 284).

¹H NMR (400 MHz, DMSO-d₆) δ (ppm): 8.34 (s, 1H), 7.66 (d, J=1.2 Hz, 1H), 7.56 (d, J=9.2 Hz, 1H), 7.50-7.43 (m, 3H), 7.35 (t, J_{HF}=74.0 Hz, 1H), 7.33 (d, J=8.8 Hz, 2H), 7.25 (dd, J=9.2 Hz, 2.0 Hz, 1H), 6.28 (tt, J_{HF}=54.0 Hz, J=3.2 Hz, 1H), 5.92 (s, 1H), 4.93 (s, 2H), 4.17 (s, 3H), 4.09 (td, J_{HF}=14.8 Hz, J=3.2 Hz, 2H).

LC-MS (ESI): m/z 535 [M+H]⁺.

### Synthesis of 7-(2,2-difluoroethoxy)-1-(4-(difluoromethoxy)phenyl)-6-fluoro-3-(2-methyl-2H-indazol-5-yl)-3,4-dihydroquinazolin-2(1H)-one (Example 285)

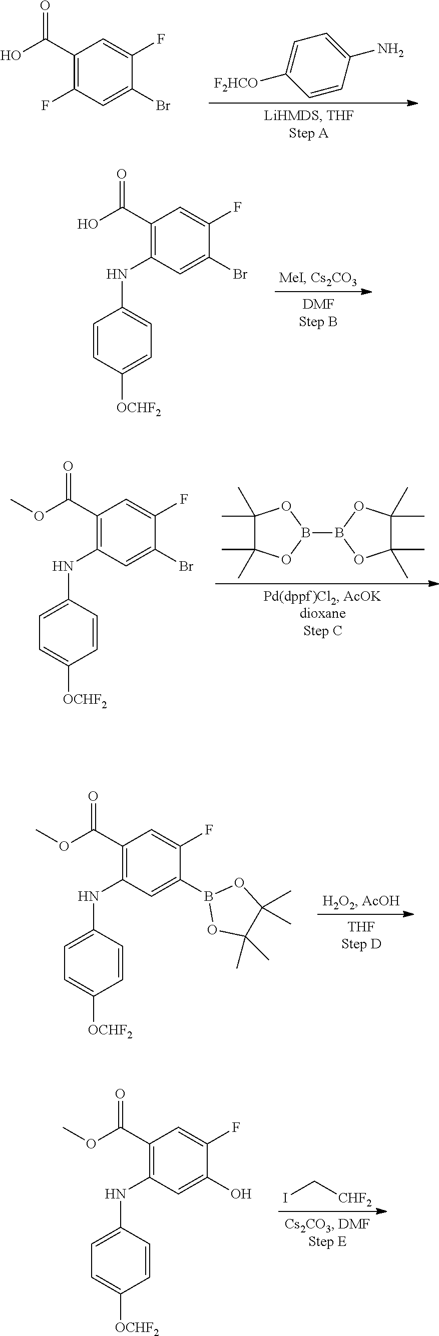

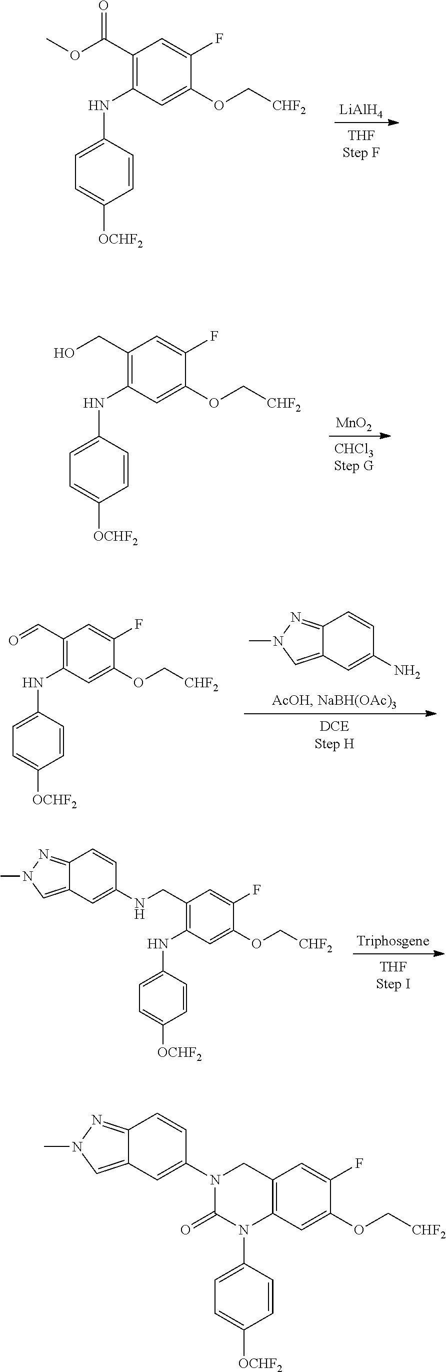

### Step A: 4-bromo-2-((4-(difluoromethoxy)phenyl)amino)-5-fluorobenzoic acid

To a solution of 4-(difluoromethoxy)aniline (269 mg, 1.6 mmol, 2.0 eq.) in THF (6 mL) was added LiHMDS (2.53 mL, 2.53 mmol, 1M, 3.2 eq.) at −78° C., after stirred for 30 min, a solution 4-bromo-2,5-difluorobenzoic acid (200 mg, 0.8 mmol, 1.0 eq.) in THF (2 mL) was added drop-wisely. The resulting mixture was allowed to warm to room temperature and stirred for 16 hrs. The progress of the reaction was monitored by LC-MS, after completion, the reaction mixture was quenched with H₂O (10 mL), the aqueous layer was adjusted pH=2 with dilute HCl (1N, aq.), extracted with EtOAc (30 mL×3), the combined organic layers were washed with brine (30 mL), dried over Na₂SO₄ and concentrated under reduced pressure, the residue was purified by flash column chromatography on silica gel to afford 4-bromo-2-((4-(difluoromethoxy)phenyl)amino)-5-fluorobenzoic acid (167 mg, 53% yield) as a white solid. LC-MS (ESI): m/z 376 [M+H]⁺.

### Step B: methyl 4-bromo-2-((4-(difluoromethoxy)phenyl)amino)-5-fluorobenzoate

To a mixture of 4-bromo-2-((4-(difluoromethoxy)phenyl)amino)-5-fluorobenzoic acid (860 mg, 2.28 mmol, 1.0 eq.) and Cs₂CO₃ (1.5 g, 4.57 mmol, 2.0 eq.) in DMF (5 mL) was added CH₃I (645 mg, 4.56 mmol, 2.0 eq.) drop-wisely at 0° C., the reaction mixture was stirred for 30 min. The progress of the reaction was monitored by LC-MS, after completion, the reaction mixture was diluted with H₂O (20 mL), extracted with EtOAc (30 mL×3), the combined organic layers were washed with brine (20 mL), dried over Na₂SO₄ and concentrated under reduced pressure, the residue was purified by flash column chromatography on silica gel to afford methyl 4-bromo-2-((4-(difluoromethoxy)phenyl)amino)-5-fluorobenzoate (780 mg, 87% yield) as a white solid.

LC-MS (ESI): m/z 390 [M+H]⁺.

### Step C: methyl 2-((4-(difluoromethoxy)phenyl)amino)-5-fluoro-4-(4,4,5,5-tetramethyl-1,3,2-dioxaborolan-2-yl)benzoate

To a mixture of methyl 4-bromo-2-{[4-(difluoromethoxy)phenyl]amino}-5-fluorobenzoate (500 mg, 1.28 mmol, 1.0 eq.) and 4,4,5,5-tetramethyl-2-(tetramethyl-1,3,2-dioxaborolan-2-yl)-1,3,2-dioxaborolane (488 mg, 1.92 mmol, 1.5 eq.) in dioxane (6 mL) was added KOAc (354 mg, 2.56 mmol, 2.0 eq.) and Pd(dppf)Cl₂ (87 mg, 0.12 mmol, 0.1 eq.), the reaction mixture was stirred at 90° C. under N₂ atmosphere for 4 hrs. The reaction mixture was diluted with H₂O (20 mL), extracted with EtOAc (20 mL×3), the combined organic layers were washed with brine (20 mL), dried over Na₂SO₄, concentrated under reduced pressure, the residue was purified by flash column chromatography on silica gel to afford methyl 2-((4-(difluoromethoxy)phenyl)amino)-5-fluoro-4-(4,4,5,5-tetramethyl-1,3,2-dioxaborolan-2-yl)benzoate (518 mg, 92% yield) as a colorless oil. LC-MS (ESI): m/z 438 [M+H]⁺.

### Step D: methyl 2-((4-(difluoromethoxy)phenyl)amino)-5-fluoro-4-hydroxybenzoate

To a solution of methyl 2-{[4-(difluoromethoxy)phenyl]amino}-5-fluoro-4-(tetramethyl-1,3,2-dioxaborolan-2-yl)benzoate (518 mg, 1.18 mmol, 1.0 eq.) in THF (4 mL) was added AcOH (0.2 mL) and H₂O₂ (30%, 1 mL), the reaction mixture was stirred at 25° C. for 1 hr. The reaction was completed as detected by TLC (Petroleum ether/EtOAc=10:1). After completion, excess of H₂O₂ was quenched with Na₂SO₃ (sat. aq.) (10 mL) at 0° C., extracted with EtOAc (20 mL×3), the combined organic layers were washed with brine (20 mL), dried over Na₂SO₄ and concentrated under reduced pressure, the residue was purified by flash column chromatography on silica gel to afford methyl 2-((4-(difluoromethoxy)phenyl)amino)-5-fluoro-4-hydroxybenzoate (310 mg, 80% yield). LC-MS (ESI): m/z 328 [M+H]⁺.

### Step E: methyl 4-(2,2-difluoroethoxy)-2-((4-(difluoromethoxy)phenyl)amino)-5-fluorobenzoate

To a mixture of methyl 2-{[4-(difluoromethoxy)phenyl]amino}-5-fluoro-4-hydroxybenzoate (310 mg, 0.94 mmol, 1.0 eq.) and Cs₂CO₃ (621 mg, 1.89 mmol, 2.0 eq) in DMF (4 mL) was added 1,1-difluoro-2-iodoethane (364 mg, 1.89 mmol, 2.0 eq.), the reaction mixture was stirred at 100° C. for 2 hrs. The progress of the reaction was monitored by LC-MS, after completion, the reaction mixture was diluted with H₂O (20 mL), extracted with EtOAc (15 mL×3), the combined organic layers were washed with brine (10 mL), dried over Na₂SO₄ and concentrated under reduced pressure, the residue was purified by flash column chromatography on silica gel to afford methyl 4-(2,2-difluoroethoxy)-2-((4-(difluoromethoxy)phenyl)amino)-5-fluorobenzoate (258 mg, 70% yield) as a white solid. LC-MS (ESI): m/z 392 [M+H]⁺.

### Step F: (4-(2,2-difluoroethoxy)-2-((4-(difluoromethoxy)phenyl)amino)-5-fluorophenyl)methanol

To a suspension of LiAlH₄ (76 mg, 1.99 mmol, 3.0 eq.) in THF (3 mL) was added a solution of methyl 4-(2,2-difluoroethoxy)-2-((4-(difluoromethoxy)phenyl)amino)-5-fluorobenzoate (260 mg, 0.66 mmol, 1.0 eq.) in THF (1 mL) drop-wisely at 0° C., the reaction mixture was allowed to warm to room temperature and stirred for 2 hrs. The progress of the reaction was monitored by LC-MS, after completion, the reaction mixture was cooled to 0° C., quenched with water (0.1 mL), aqueous NaOH (0.1 mL, 15%) and H₂O (0.3 mL) in sequence, then filtered through a short pad of Celite®, the filtrate was concentrated under reduced pressure, the residue was purified by flash column chromatography on silica gel to afford (4-(2,2-difluoroethoxy)-2-((4-(difluoromethoxy)phenyl)amino)-5-fluorophenyl)methanol (230 mg, 95% yield) as a colorless oil. LC-MS (ESI): m/z 364 [M+H]⁺.

### Step G: 4-(2,2-difluoroethoxy)-2-((4-(difluoromethoxy)phenyl)amino)-5-fluorobenzaldehyde

To a solution of (4-(2,2-difluoroethoxy)-2-((4-(difluoromethoxy)phenyl)amino)-5-fluorophenyl)methanol (250 mg, 0.69 mmol, 1.0 eq.) in CHCl₃ (5 mL) was added MnO₂ (897 mg, 10.3 mmol, 15.0 eq.) in several portions, the reaction mixture was stirred at 40° C. for 16 hrs. The progress of the reaction was monitored by LC-MS, after completion, excess of MnO₂ was filtered through a short pad of Celite®, the filtrate was concentrated under reduced pressure, the residue was purified by flash column chromatography on silica gel to afford 4-(2,2-difluoroethoxy)-2-((4-(difluoromethoxy)phenyl)amino)-5-fluorobenzaldehyde (70 mg, 28% yield) as a white solid. LC-MS (ESI): m/z 362 [M+H]⁺.

### Step H: N-(4-(2,2-difluoroethoxy)-2-((4-(difluoromethoxy)phenyl)amino)-5-fluorobenzyl)-2-methyl-2H-indazol-5-amine

To a solution of -(2,2-difluoroethoxy)-2-((4-(difluoromethoxy)phenyl)amino)-5-fluorobenzaldehyde (44 mg, 0.12 mmol, 1.0 eq.) in DCE (10 mL) was added 2-methyl-2H-indazol-5-amine (22 mg, 0.15 mmol, 1.2 eq.) and AcOH (29 mg, 0.49 mmol, 4.0 eq.), the reaction mixture was stirred at room temperature for 15 hrs. Then the reaction mixture was cooled to 0° C., NaBH(OAc)₃ (77 mg, 0.36 mmol, 3.0 eq.) was added in one portion, the reaction mixture was allowed to warm to room temperature and stirred for 3 hrs. The progress of the reaction was monitored by LC-MS, after completion, the reaction was quenched with ice water (10 mL) and extracted with DCM (20 mL×3). The combined organic layers were washed with brine (20 mL), dried over Na₂SO₄ and concentrated under reduced pressure, the residue was purified by column chromatography to afford N-(4-(2,2-difluoroethoxy)-2-((4-(difluoromethoxy)phenyl)amino)-5-fluorobenzyl)-2-methyl-2H-indazol-5-amine (48 mg, 80% yield) as a pale green solid. LC-MS(ESI): m/z 493 [M+H]⁺.

### Step I: 7-(2,2-difluoroethoxy)-1-(4-(difluoromethoxy)phenyl)-6-fluoro-3-(2-methyl-2H-indazol-5-yl)-3,4-dihydroquinazolin-2(1H)-one

To a solution of N-(4-(2,2-difluoroethoxy)-2-((4-(difluoromethoxy)phenyl)amino)-5-fluorobenzyl)-2-methyl-2H-indazol-5-amine (50 mg, 0.1 mmol, 1.0 eq.) in THF (2 mL) was added triphosgene (30 mg, 0.1 mmol, 1.0 eq.) at 0° C., the reaction mixture was allowed to warm to room temperature and stirred for 1 hr. The progress of the reaction was monitored by LC-MS, after completion, the reaction mixture was quenched with ice cooled NaHCO₃ (sat. aq.) (10 mL), extracted with EtOAc (20 mL×3). The combined organic layers were washed with brine (20 mL), dried over Na₂SO₄ and concentrated under reduced pressure, the residue was purified by RP-prep-HPLC to afford 7-(2,2-difluoroethoxy)-1-(4-(difluoromethoxy)phenyl)-6-fluoro-3-(2-methyl-2H-indazol-5-yl)-3,4-dihydroquinazolin-2(1H)-one (Example 285).

¹H NMR (400 MHz, DMSO-d₆) δ (ppm): 8.34 (s, 1H), 7.65 (s, 1H), 7.55 (d, J=9.2 Hz, 1H), 7.46 (d, J=8.8 Hz, 2H), 7.35 (t, J_{HF}=74.0 Hz, 1H), 7.34-7.24 (m, 4H), 6.27 (tt, J_{HF}=54.0 Hz, 3.2 Hz, 1H), 5.94 (d, J_{HF}=7.2 Hz, 1H), 4.93 (s, 2H), 4.16 (s, 3H), 4.13 (td, J_{HF}=14.8 Hz, J=3.2 Hz, 2H).

LC-MS (ESI): m/z 519 [M+H]⁺.

### Synthesis of 7-(2,2-difluoroethoxy)-1-(4-(methoxy-d3)phenyl)-4-methyl-3-(2-methyl-2H-indazol-5-yl)-3,4-dihydropyrido[2,3-d]pyrimidin-2(1H)-one (Example 286)

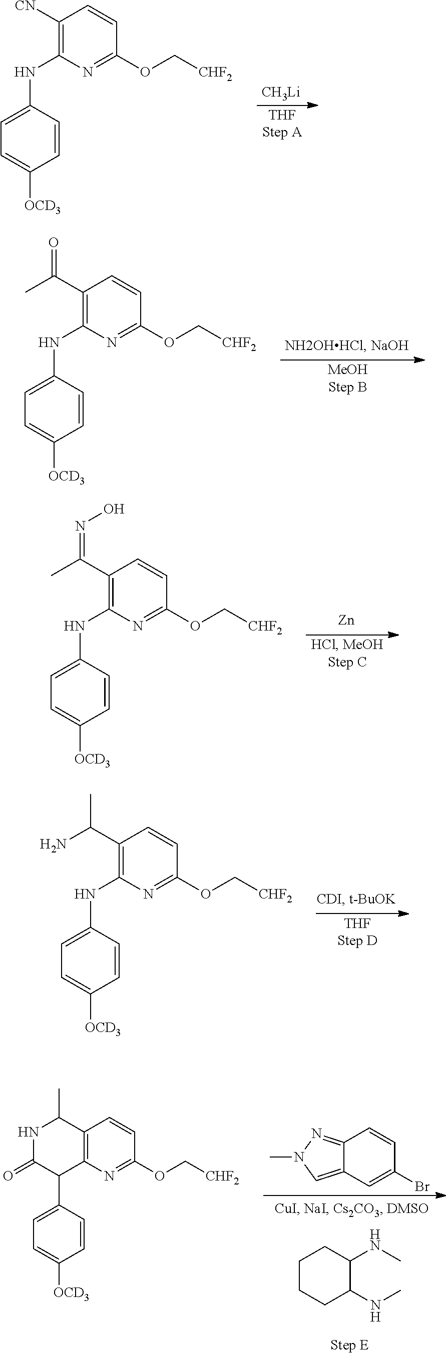

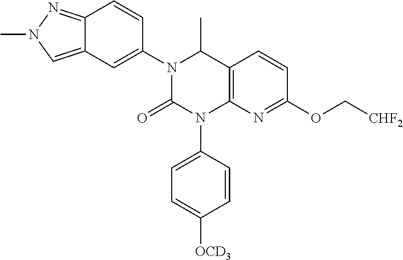

### Step A: 1-(6-(2,2-difluoroethoxy)-2-((4-(methoxy-d3)phenyl)amino)pyridin-3-yl)ethan-1-one

To a solution of 6-(2,2-difluoroethoxy)-3-isocyano-N-(4-(methoxy-d3)phenyl)pyridin-2-amine (500 mg, 1.62 mmol, 1.0 eq.) (prepared via General Procedure I, Steps A-B) in THF (20 mL) was added CH₃Li (5.1 mL, 8.1 mmol, 1.6 M, 5.0 eq.) drop-wisely at 0° C. under N₂ atmosphere, the reaction mixture was stirred for 2 hrs. The reaction was monitored by TLC. After completion, the reaction was quenched with H₂O (10 mL), extracted with EtOAc (20 mL×3), the combined organic layers were washed with brine (10 mL) and dried over Na₂SO₄ and concentrated under reduced pressure, the residue was purified by flash column chromatography to afford 1-(6-(2,2-difluoroethoxy)-2-((4-(methoxyd3)phenyl)amino)pyridin-3-yl)ethan-1-one (250 mg, 48%) as a yellow solid. LC-MS (ESI): m/z 326.1 [M+H]⁺.

### Step B: 1-(6-(2,2-difluoroethoxy)-2-((4-(methoxy-d3)phenyl)amino)pyridin-3-yl)ethan-1-one oxime

To a mixture of 1-(6-(2,2-difluoroethoxy)-2-((4-(methoxy-d3)phenyl)amino)pyridin-3-yl)ethan-1-one (250 mg, 0.77 mmol, 1.0 eq.) and hydroxylamine hydrochloride (267 mg, 3.85 mmol, 5.0 eq.) in MeOH (5 mL) was added NaOH (215 mg, 5.39 mmol, 7.0 eq.), the reaction mixture was stirred at 60° C. for 6 hrs. The reaction mixture was quenched with H₂O (20 mL) and extracted with EA (20 mL×3), the combined organic layers were washed with brine (10 mL) and dried over Na₂SO₄, concentrated under reduced pressure and purified by flash column chromatography to afford 1-(6-(2,2-difluoroethoxy)-2-((4-(methoxy-d3)phenyl)amino)pyridin-3-yl)ethan-1-oneoxime (220 mg, 84% yield) as a yellow solid. LC-MS (ESI): m/z 341.1 [M+H]⁺.

### Step C: 3-(1-aminoethyl)-6-(2,2-difluoroethoxy)-N-(4-(methoxy-d3)phenyl)pyridin-2-amine

To a mixture of 1-(6-(2,2-difluoroethoxy)-2-((4-(methoxy-d3)phenyl)amino)pyridin-3-yl)ethan-1-oneoxime (220 mg, 0.65 mmol, 1.0 equiv.) and Zn (420 mg, 6.50 mmol, 10 eq.) in MeOH (15 mL) was added concentrated hydrochloric acid (1.0 mL) drop-wisely at 60° C., the reaction mixture was stirred at 60° C. for 3 hrs. Then the reaction mixture was cooled to 0° C., diluted with H₂O (20 mL) and filtered, the filter cake was washed with EtOAc (10 mL×3), the filtrate was extracted with EtOAc (20 mL×3). The combined organic layers were washed with brine (30 mL), dried over Na₂SO₄ and concentrated under reduced pressure, the residue was purified by flash column chromatography to afford 3-(1-aminoethyl)-6-(2,2-difluoroethoxy)-N-(4-(methoxy-d3)phenyl)pyridin-2-amine (170 mg, 81% yield) as a blown oil. LC-MS (ESI): m/z 327.2 [M+H]⁺.

### Step D: 7-(2,2-difluoroethoxy)-1-(4-(methoxy-d3)phenyl)-4-methyl-3,4-dihydropyrido[2,3-d]pyrimidin-2(1H)-one

To a solution of 3-(1-aminoethyl)-6-(2,2-difluoroethoxy)-N-(4-(methoxy-d3)phenyl)pyridin-2-amine (170 mg, 0.52 mmol, 1.0 eq.) in THF (10 mL) was added CDI (169 mg, 1.04 mmol, 2.0 eq.) and t-BuOK (117 mg, 1.04 mmol, 2.0 eq.), the reaction mixture was stirred at 65° C. for 2 hrs. Then the reaction mixture was quenched with H₂O (20 mL) and extracted with EtOAc (20 mL×3), the combined organic layers were washed with brine (10 mL), dried over Na₂SO₄ and concentrated under reduced pressure, the residue was purified by flash column chromatography to afford 7-(2,2-difluoroethoxy)-1-(4-(methoxy-d3)phenyl)-4-methyl-3,4-dihydropyrido[2,3-d]pyrimidin-2(1H)-one (130 mg, 71% yield) as a white solid.

LC-MS (ESI): m/z=353.1 [M+H]⁺.

### Step E: 7-(2,2-difluoroethoxy)-1-(4-(methoxy-d3)phenyl)-4-methyl-3-(2-methyl-2H-indazol-5-yl)-3,4-dihydropyrido[2,3-d]pyrimidin-2(1H)-one

To a solution of 7-(2,2-difluoroethoxy)-1-(4-(methoxy-d3)phenyl)-4-methyl-3,4-dihydropyrido[2,3-d]pyrimidin-2(1H)-one (100 mg, 0.28 mmol, 1.0 eq.) in DMSO (6 mL) was added 5-bromo-2-methyl-2H-indazole (418 mg, 1.99 mmol, 7.0 eq.), N¹,N²-dimethylcyclohexane-1,2-diamine (81 mg, 0.57 mmol, 2.0 eq.), CuI (109 mg, 0.57 mmol, 2.0 eq.), NaI (85 mg, 0.57 mmol, 2.0 eq.) and Cs₂CO₃ (185 mg, 0.57 mmol, 2.0 eq.), the reaction mixture was stirred at 140° C. for 8 hrs and at 110° C. for 15 hrs under N₂ atmosphere. The reaction was diluted with H₂O (15 mL) and extracted with EtOAc (20 mL×3), the combined organic layers were washed with brine (20 mL), dried over Na₂SO₄ and concentrated under reduced pressure, the residue was purified by RP-Prep-HPLC to afford 7-(2,2-difluoroethoxy)-1-(4-(methoxy-d3)phenyl)-4-methyl-3-(2-methyl-2H-indazol-5-yl)-3,4-dihydropyrido[2,3-d]pyrimidin-2(1H)-one.

¹H NMR (400 MHz, DMSO-d₆) δ (ppm): 8.35 (s, 1H), 7.73-7.66 (m, 2H), 7.60 (d, J=8.8 Hz, 1H), 7.25 (d, J=8.8 Hz, 2H), 7.20 (dd, J=9.2 Hz, 1.6 Hz, 1H), 6.99 (d, J=8.8 Hz, 2H), 6.54 (d, J=8.4 Hz, 1H), 6.02 (tt, J_{HF}=55.6 Hz, J=4.0 Hz, 1H), 5.04 (q, J=6.4 Hz, 1H), 4.17 (s, 3H), 4.15-3.97 (m, 2H), 1.44 (d, J=6.4 Hz, 3H).

LC-MS (ESI): m/z=483.2 [M+H]⁺.

### Synthesis of 1-(4-methoxyphenyl)-3-(2-methyl-2H-indazol-5-yl)-7-propyl-3,4-dihydropyrido[2,3-d]pyrimidin-2(1H)-one (Example 294)

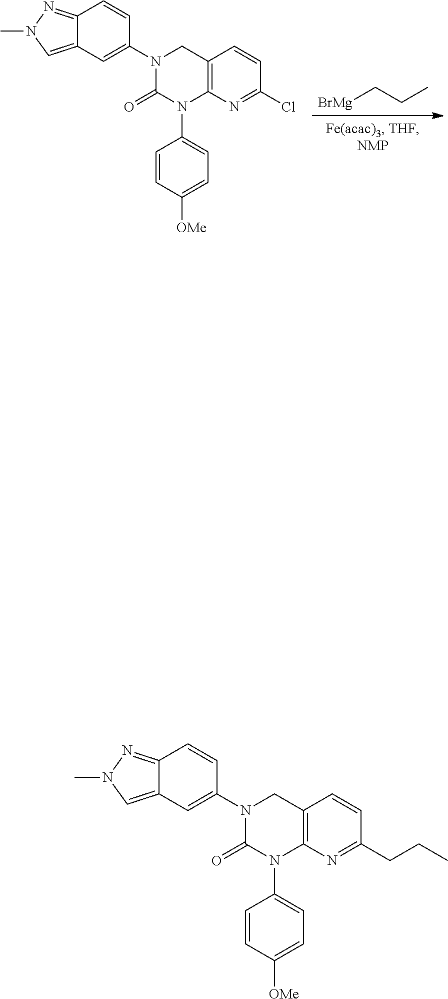

To a mixture of 7-chloro-1-(4-methoxyphenyl)-3-(2-methyl-2H-indazol-5-yl)-3,4-dihydropyrido[2,3-d]pyrimidin-2(1H)-one (109 mg, 0.26 mmol, 1.0 eq.) and Fe(acac)₃ (93 mg, 0.26 mmol, 1.0 eq.) in THF (5 mL) and NMP (1 mL) was added n-propylmagnesium bromide (1 M in diethyl ether, 4.0 mL, 4.0 mmol, 15.4 eq.) at 0° C. under N₂ atmosphere dropwise. The mixture was stirred at room temperature overnight and quenched with ice water (10 mL) carefully. The crude mixture was extracted with EtOAc (10 mL×3). The combined organic layers were washed with brine (20 mL) and dried over Na₂SO₄, concentrated under reduced pressure. The crude residue was purified by RP-prep-HPLC to give 1-(4-methoxyphenyl)-3-(2-methyl-2H-indazol-5-yl)-7-propyl-3,4-dihydropyrido[2,3-d]pyrimidin-2(1H)-one (Example 294).

¹H NMR (400 MHz, DMSO-d₆) δ: 8.34 (s, 1H), 7.67 (d, J=1.5 Hz, 1H), 7.56 (dd, J=8.3 Hz, 3.8 Hz, 2H), 7.26 (dd, J=9.2 Hz, 2.0 Hz, 1H), 7.19 (d, J=8.8 Hz, 2H), 6.97 (d, J=8.8 Hz, 2H), 6.87 (d, J=7.5 Hz, 1H), 4.94 (s, 2H), 4.17 (s, 3H), 3.80 (s, 3H), 2.43 (t, J=7.5 Hz, 2H), 1.54-1.37 (m, 2H), 0.79 (t, J=7.3 Hz, 3H).

LC-MS (ESI): m/z 428.1 [M+H]⁺.

### Synthesis of 7-(2,2-difluoroethoxy)-1-(4-hydroxyphenyl)-3-(2-methyl-2H-indazol-5-yl)-3,4-dihydropyrido[2,3-d]pyrimidin-2(1H)-one (Example 295)

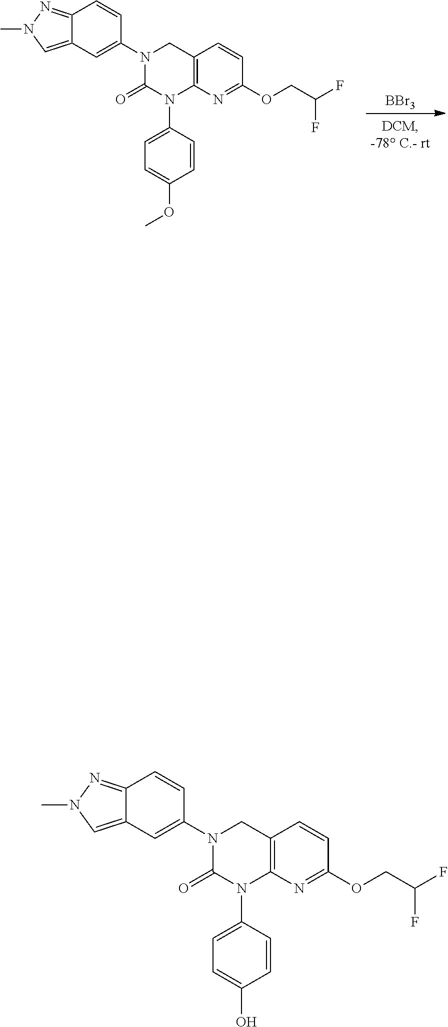

To a solution of 7-(2,2-difluoroethoxy)-1-(4-methoxyphenyl)-3-(2-methyl-2H-indazol-5-yl)-3,4-dihydropyrido[2,3-d]pyrimidin-2(1H)-one (Example 141) (150 mg, 0.32 mmol, 1.0 eq.) in DCM (4 mL) was added BBr₃ (403 mg, 1.61 mmol, 5.0 eq.) dropwise at −78° C., the reaction mixture stirred at −78° C. for 0.5 hr, then it was allowed to warm to 0° C. The reaction was quenched by adding NaHCO₃(Sat. aq) (10 mL), extracted with DCM (10 mL×3). The combined organic layers were washed with brine (20 mL), dried over Na₂SO₄ and concentrated under reduced pressure, the residue was purified by RP-prep-HPLC to afford 7-(2,2-difluoroethoxy)-1-(4-hydroxyphenyl)-3-(2-methyl-2H-indazol-5-yl)-3,4-dihydropyrido[2,3-d]pyrimidin-2(1H)-one (Example 295).

¹H NMR (400 MHz, DMSO-d₆) δ: 9.50 (s, 1H), 8.34 (s, 1H), 7.68 (d, J=1.6 Hz, 1H), 7.65 (d, J=8.4 Hz, 1H), 7.57 (d, J=9.2 Hz, 1H), 7.26 (dd, J=9.2 Hz, 2.0 Hz, 1H), 7.12 (d, J=8.8 Hz, 2H), 6.82 (d, J=8.8 Hz, 2H), 6.51 (d, J=8.0 Hz, 1H), 6.03 (tt, J_{HF}=55.6 Hz, J=4.0 Hz, 1H), 4.92 (s, 2H), 4.17 (s, 3H), 4.08 (td, J_{HF}=14.4 Hz, J=4.0 Hz, 2H).

LC-MS (ESI): m/z 452.2 [M+H]⁺.

### Synthesis of 7-(2,2-difluoroethoxy)-1-(4-methoxycyclohexyl)-3-(2-methyl-2H-indazol-5-yl)-1H,2H,3H,4H-pyrido[2,3-d]pyrimidin-2-one (Example 427)

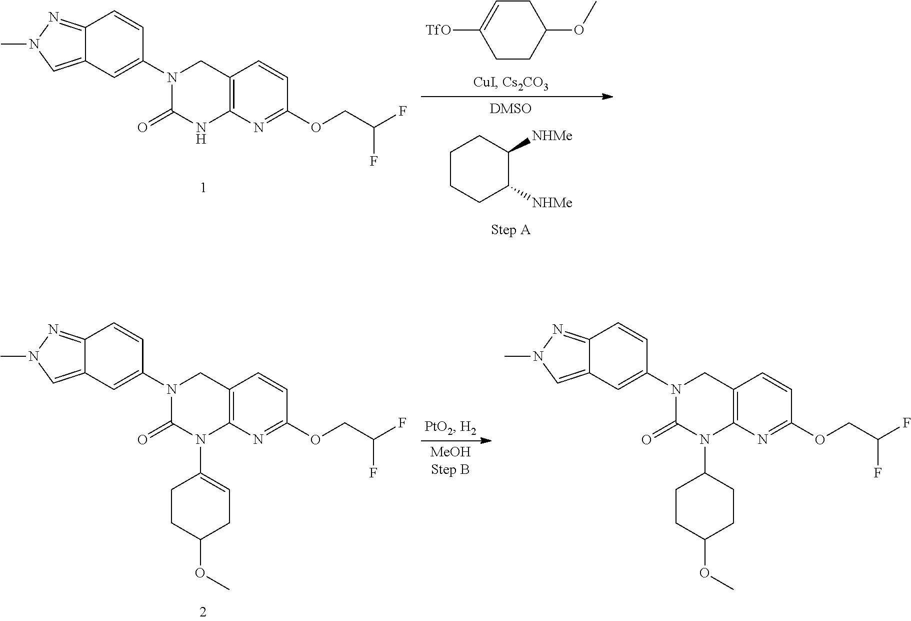 [SMILES: Cn1cc2cc(N3Cc4ccc(OCC(F)F)nc4NC3=O)ccc2n1 ; COC1CC=C(OS(=O)(=O)C(F)(F)F)CC1 ; CN[C@@H]1CCCC[C@H]1NC ; COC1CC=C(N2C(=O)N(c3ccc4nn(C)cc4c3)Cc3ccc(OCC(F)F)nc32)CC1 ; COC1CCC(N2C(=O)N(c3ccc4nn(C)cc4c3)Cc3ccc(OCC(F)F)nc32)CC1]

### Step A: 7-(2,2-difluoroethoxy)-1-(4-methoxycyclohex-1-en-1-yl)-3-(2-methyl-2H-indazol-5-yl)-1H,2H,3H,4H-pyrido[2,3-d]pyrimidin-2-one

To a solution of 7-(2,2-difluoroethoxy)-3-(2-methyl-2H-indazol-5-yl)-1H,2H,3H,4H-pyrido[2,3-d]pyrimidin-2-one (400 mg, 1.11 mmol, 1.0 eq.) in DMSO (8 mL) was added Cs₂CO₃ (1.09 g, 3.33 mmol, 3.0 eq.), CuI (212 mg, 1.11 mmol, 1.0 eq.), (1R,2R)—N1,N2-dimethylcycloh,exane-1,2-diamine (315 mg, 2.22 mmol, 2.0 eq.) and 4-methoxycyclohex-1-en-1-yl trifluoromethanesulfonate (Ref: J. Am. Chem. Soc., 2018, 140, 2446-2449) (579 mg 2.22 mmol, 2.0 eq.), the mixture was stirred at 100° C. under N₂ atmosphere for 12 hrs. After completion, the reaction was quenched with water (50 mL) and extracted with EtOAc (80 mL×3). The combined organic layers were dried over Na₂SO₄, filtered and concentrated under reduced pressure, the residue was purified by flash column chromatography on silica gel to give 7-(2,2-difluoroethoxy)-1-(4-methoxycyclohex-1-en-1-yl)-3-(2-methyl-2H-indazol-5-yl)-1H,2H,3H,4H-pyrido[2,3-d]pyrimidin-2-one (480 mg, 92%) as a yellow solid. LC-MS (ESI): m/z 470 [M+H]⁺.

### Step B: 7-(2,2-difluoroethoxy)-1-(4-methoxycyclohexyl)-3-(2-methyl-2H-indazol-5-yl)-1H,2H,3H,4H-pyrido[2,3-d]pyrimidin-2-one

To a solution of 7-(2,2-difluoroethoxy)-1-(4-methoxycyclohex-1-en-1-yl)-3-(2-methyl-2H-indazol-5-yl)-1H,2H,3H,4H-pyrido[2,3-d]pyrimidin-2-one (120 mg, 0.25 mmol, 1.0 eq.) in MeOH (8 mL) was added PtO₂ (12 mg, 0.05 mmol, 0.2 eq.), the reaction mixture was degassed with H₂ and stirred at 50° C. under H₂ atmosphere (1 atm) for 12 hrs. The reaction was complete as indicated by LCMS. The reaction mixture was filtered through a short pad of Celite®, the filtrate was concentrated under reduced pressure, the residue was purified by RP-prep-HPLC to give 7-(2,2-difluoroethoxy)-1-(4-methoxycyclohexyl)-3-(2-methyl-2H-indazol-5-yl)-1H,2H,3H,4H-pyrido[2,3-d]pyrimidin-2-one (Example 427).

¹H NMR (400 MHz, DMSO-d₆) δ (ppm): 8.30 (s, 1H), 7.57 (d, J=8.5 Hz, 1H), 7.57 (s, 1H), 7.53 (d, J=9.1 Hz, 1H), 7.16 (d, J=9.2 Hz, 1H), 6.50 (d, J=8.0 Hz, 1H), 6.40 (tt, J_{HF}=55.5 Hz, J=3.8 Hz, 1H), 4.69 (s, 2H), 4.62 (td, J_{HF}=14.9 Hz, J=3.5 Hz, 2H), 4.55-4.45 (m, 1H), 4.15 (s, 3H), 3.22 (s, 3H), 3.24-3.18 (m, 1H), 2.90-2.72 (m, 2H), 2.01-1.96 (m, 2H), 1.50-1.38 (m, 4H).

LC-MS (ESI): m/z 472.6 [M+H]⁺.

### Synthesis of 1-(4-(difluoromethoxy)phenyl)-3-(2-methyl-2H-indazol-5-yl)-7-((2,2,2-trifluoroethyl)amino)-3,4-dihydropyrido[3,2-d]pyrimidin-2(1H)-one (Example 428)

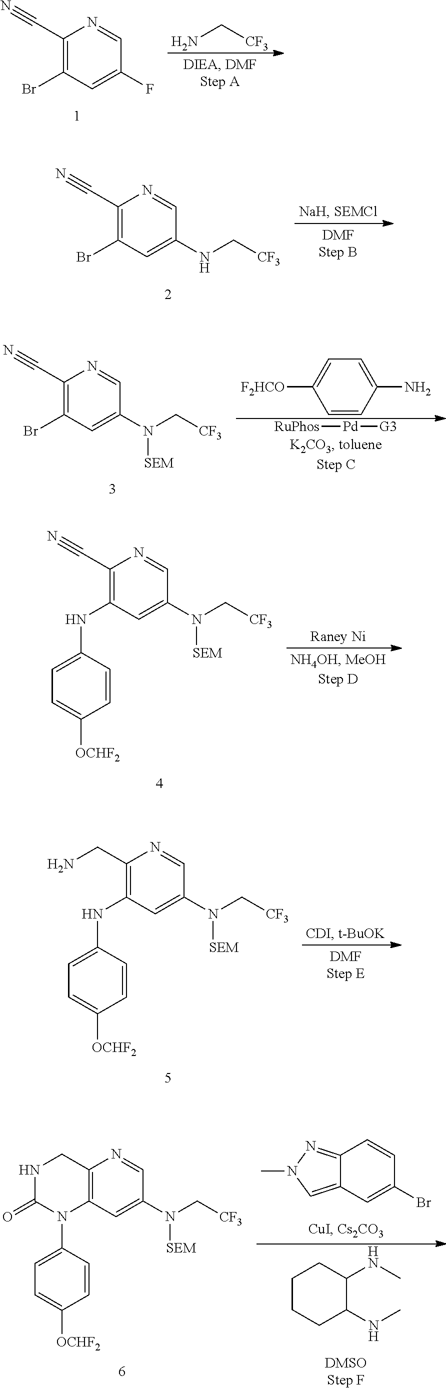

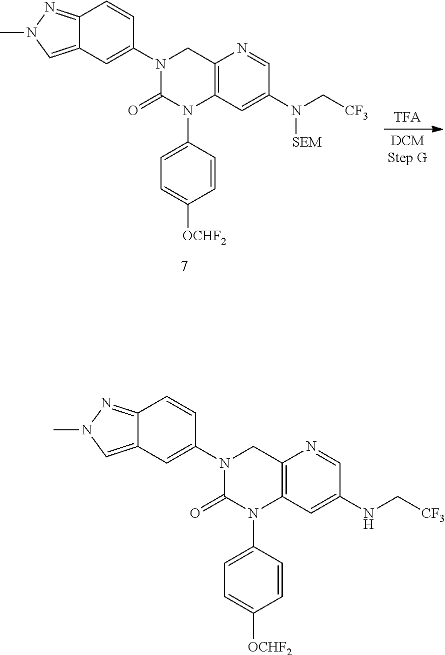 [SMILES: Cn1cc2cc(N3Cc4ncc(N(COCC[Si](C)(C)C)CC(F)(F)F)cc4N(c4ccc(OC(F)F)cc4)C3=O)ccc2n1 ; Cn1cc2cc(N3Cc4ncc(NCC(F)(F)F)cc4N(c4ccc(OC(F)F)cc4)C3=O)ccc2n1]

### Step A: 3-bromo-5-((2,2,2-trifluoroethyl)amino)picolinonitrile

To a solution of 3-bromo-5-fluoropicolinonitrile (5.0 g, 24.9 mmol, 1.0 eq.) and DIEA (9.7 g, 74.6 mmol, 3.0 eq.) in DMF (50 mL) was added 2,2,2-trifluoroethan-1-amine (7.4 g, 74.6 mmol, 3.0 eq.), the reaction mixture was sealed in a tube and stirred at 100° C. for 15 hrs. Then the reaction mixture was poured into H₂O (50 mL) and extracted with EtOAc (50 mL×3). The combined organic layers were washed with brine (30 mL), dried over Na₂SO₄ and concentrated under reduced pressure, the residue was purified by flash column chromatography on silica gel to give 3-bromo-5-((2,2,2-trifluoroethyl)amino)picolinonitrile (4.5 g, 65% yield) as a pale yellow solid. LC-MS (ESI): m/z 280, 282 [M+H]⁺.

### Step B: 3-bromo-5-((2,2,2-trifluoroethyl)((2-(trimethylsilyl)ethoxy)methyl)amino)picolinonitrile

To a solution of 3-bromo-5-((2,2,2-trifluoroethyl)amino)picolinonitrile (1.13 g, 4.04 mmol, 1.0 eq.) in DMF (10 mL) was added NaH (60% wt suspend in mineral oil) (323 mg, 8.07 mmol, 2.0 eq.) in several portions at 0° C., the reaction mixture was stirred at room temperature for 0.5 hr. Then the reaction mixture was cooled to 0° C. and SEMCI (807 mg, 4.84 mmol, 1.2 eq.) was added dropwise, the reaction mixture was allowed warm to room temperature and stirred for additional 2 hrs. After completion, the reaction mixture was poured into ice water (30 mL), extracted with EtOAc (40 mL×3). The combined organic layers were washed with brine (20 mL), dried over Na₂SO₄ and concentrated under reduced pressure, the residue was purified by flash column chromatography on silica gel to give 3-bromo-5-((2,2,2-trifluoroethyl)((2-(trimethylsilyl)ethoxy)methyl)amino)picolinonitrile (1.1 g, 66%) as a pale yellow oil. LC-MS (ESI): m/z 410, 412 [M+H]⁺.

### Step C: 3-((4-(difluoromethoxy)phenyl)amino)-5-((2,2,2-trifluoroethyl)((2-(trimethylsilyl)ethoxy)methyl)amino)picolinonitrile

A mixture of 3-bromo-5-((2,2,2-trifluoroethyl)((2-(trimethylsilyl)ethoxy)methyl)amino)picolinonitrile (400 mg, 0.97 mmol, 1.0 eq.), 4-(difluoromethoxy)aniline (155 mg, 0.97 mmol, 1.0 eq.), RuPhos-Pd-G3 precatalyst (81 mg, 0.097 mmol, 0.1 eq.) and K₂CO₃ (269 mg, 1.95 mmol, 2.0 eq.) in toluene (5 mL) was stirred at 100° C. for 15 hrs. Then the reaction mixture was diluted with H₂O (15 mL) and extracted with EtOAc (20 mL×3), the combined organic layers were washed with brine (15 mL), dried over Na₂SO₄ and concentrated under reduced pressure, the residue was purified by flash column chromatography on silica gel to give 3-((4-(difluoromethoxy)phenyl)amino)-5-((2,2,2-trifluoroethyl)((2-(trimethylsilyl)ethoxy)methyl)amino)picolinonitrile (200 mg, 42%) as a pale yellow oil. LC-MS (ESI): m/z 489 [M+H]⁺.

### Step D: 2-(aminomethyl)-N3-(4-(difluoromethoxy)phenyl)-N5-(2,2,2-trifluoroethyl)-N5-((2-(trimethylsilyl)ethoxy)methyl)pyridine-3,5-diamine

To a solution of 3-((4-(difluoromethoxy)phenyl)amino)-5-((2,2,2-trifluoroethyl)((2-(trimethylsilyl)ethoxy)methyl)amino)picolinonitrile (90 mg, 0.18 mmol, 1.0 eq.) in MeOH (3 mL) was added Raney Ni (30 mg), the reaction mixture was degassed with H₂, then NH₄OH (1 mL) was added, the reaction mixture was stirred at room temperature under H₂ atmosphere (1 atm) for 5 hours. The reaction mixture was filtered through a short pad of Celite®, the filtrate was concentrated under reduced pressure to give 2-(aminomethyl)-N3-(4-(difluoromethoxy)phenyl)-N5-(2,2,2-trifluoroethyl)-N5-((2-(trimethylsilyl)ethoxy)methyl)pyridine-3,5-diamine (90 mg, crude) as a brown oil, which was used for next step without further purification. LC-MS (ESI): m/z 493 [M+H]⁺.

### Step E: 1-4-(difluoromethoxy)phenyl)-7-((2,2,2-trifluoroethyl)((2-(trimethylsilyl)ethoxy)methyl)amino)-3,4-dihydropyrido[3,2-d]pyrimidin-2(1H)-one

To a solution of 2-(aminomethyl)-N3-(4-(difluoromethoxy)phenyl)-N5-(2,2,2-trifluoroethyl)-N5-((2-(trimethylsilyl)ethoxy)methyl)pyridine-3,5-diamine (90 mg, 0.18 mmol, 1.0 eq.) in DMF (2 mL) was added CDI (89 mg, 0.54 mmol, 3.0 eq.) and t-BuOK (82 mg, 0.72 mmol, 4.0 eq.), the reaction mixture was stirred at 50° C. for 5 hrs. Then the reaction mixture was poured into ice water (10 mL) and extracted with EtOAc (20 mL×3), the combined organic layers were washed with brine (15 mL), dried over Na₂SO₄ and concentrated under reduced pressure, the residue was purified by flash column chromatography on silica gel to give 1-(4-(difluoromethoxy)phenyl)-7-((2,2,2-trifluoroethyl)((2-(trimethylsilyl)ethoxy)methyl)amino)-3,4-dihydropyrido[3,2-d]pyrimidin-2(1H)-one (52 mg, 53%) as a white solid. LC-MS (ESI): m/z 519 [M+H]⁺.

### Step F: 1-(4-(difluoromethoxy)phenyl)-3-(2-methyl-2H-indazol-5-yl)-7-((2,2,2-trifluoroethyl)((2-(trimethylsilyl)ethoxy)methyl)amino)-3,4-dihydropyrido[3,2-d]pyrimidin-2(1H)-one

A mixture of 1-(4-(difluoromethoxy)phenyl)-7-((2,2,2-trifluoroethyl)((2-(trimethylsilyl)ethoxy)methyl)amino)-3,4-dihydropyrido[3,2-d]pyrimidin-2(1H)-one (52 mg, 0.1 mmol, 1.0 eq.), 5-bromo-2-methyl-2H-indazole (32 mg, 0.15 mmol, 1.5 eq.), N1,N2-dimethylcyclohexane-1,2-diamine (29 mg, 0.2 mmol, 2.0 eq.), CuI (19 mg, 0.1 mmol, 1.0 eq.) and CsF (46 mg, 0.3 mmol, 3.0 eq.) in DMSO (1.5 mL) was degassed with N₂ and stirred at 100° C. under N₂ atmosphere for 3 hrs. Then the reaction mixture was diluted with water (10 mL) and extracted with EtOAc (20 mL×3), the combined organic layers were washed with brine (15 mL), dried over Na₂SO₄ and concentrated under reduced pressure, the residue was purified by flash column chromatography on silica gel to give 1-(4-(difluoromethoxy)phenyl)-3-(2-methyl-2H-indazol-5-yl)-7-((2,2,2-trifluoroethyl)((2-(trimethylsilyl)ethoxy)methyl)amino)-3,4-dihydropyrido[3,2-d]pyrimidin-2(1H)-one (18 mg, 28%) as a white solid. LC-MS (ESI): m/z 649 [M+H]⁺.

### Step G: 1-(4-(difluoromethoxy)phenyl)-3-(2-methyl-2H-indazol-5-yl)-7-((2,2,2-trifluoroethyl)amino)-3,4-dihydropyrido[3,2-d]pyrimidin-2(1H)-one

To a solution of 1-(4-(difluoromethoxy)phenyl)-3-(2-methyl-2H-indazol-5-yl)-7-((2,2,2-trifluoroethyl)((2-(trimethylsilyl)ethoxy)methyl)amino)-3,4-dihydropyrido[3,2-d]pyrimidin-2(1H)-one (18 mg, 0.028 mmol, 1.0 eq.) in DCM (1 mL) was added TFA (0.5 mL), the reaction mixture was stirred at room temperature for 5 hrs. Then the reaction mixture was concentrated under reduced pressure, the residue was diluted with DCM (20 mL), washed with NaHCO₃ (sat. aq.) (10 mL), concentrated under reduced pressure, the residue was purified by RP-prep-HPLC to give 1-(4-(difluoromethoxy)phenyl)-3-(2-methyl-2H-indazol-5-yl)-7-((2,2,2-trifluoroethyl)amino)-3,4-dihydropyrido[3,2-d]pyrimidin-2(1H)-one (Example 428)

¹H NMR (400 MHz, DMSO-d₆) δ (ppm): 8.33 (s, 1H), 7.75 (s, 1H), 7.70 (s, 1H), 7.56 (d, J=9.2 Hz, 1H), 7.45 (d, J=8.5 Hz, 2H), 7.35 (t, J_{HF}=75.4 Hz, 1H), 7.34 (d, J=8.5 Hz, 2H), 7.28 (d, J=9.2 Hz, 1H), 6.44 (t, J=6.8 Hz, 1H), 5.94 (s, 1H), 4.90 (s, 2H), 4.16 (s, 3H), 3.92-3.80 (m, 2H).

LC-MS (ESI): m/z 519 [M+H]⁺.

### Synthesis of 7-(2,2-difluoroethoxy)-1-(trans-4-methoxycyclohexyl)-3-(2-methyl-2H-indazol-5-yl)-3,4-dihydropyrido[2,3-d]pyrimidin-2(1H)-one (Example 429)

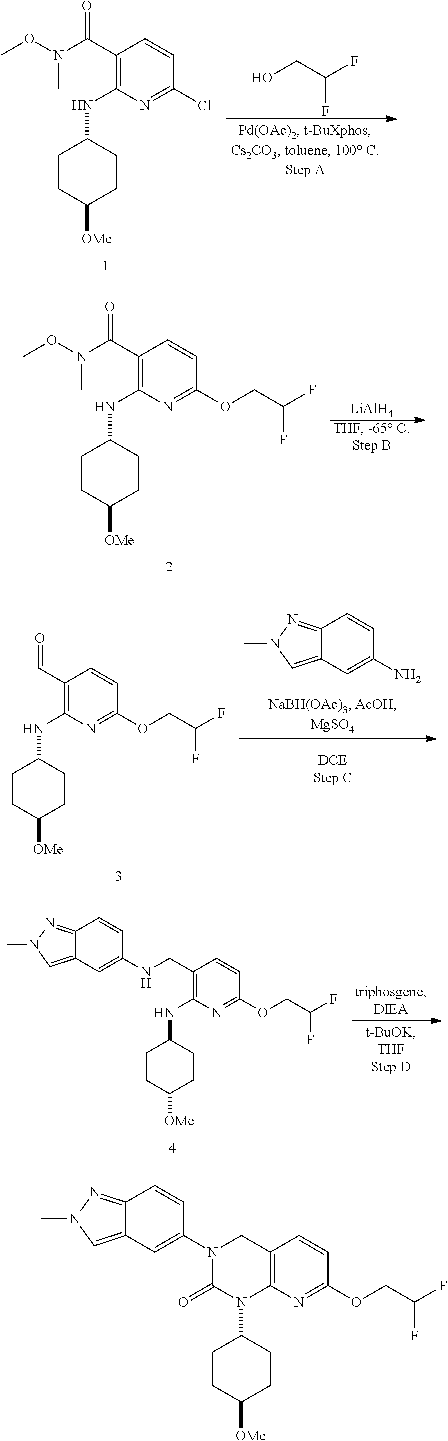

### Step A: 6-(2,2-difluoroethoxy)-N-methoxy-2-((trans-4-methoxycyclohexyl)amino)-N-methylnicotinamide

To a solution of 6-chloro-N-methoxy-2-((trans-4-methoxycyclohexyl)amino)-N-methylnicotinamide (synthesized from 2,6-dichloronicotinic acid&trans-4-methoxycyclohexan-1-amine via Urea General Procedure III (Step A&B)) (210 mg, 0.64 mmol, 1.0 eq.) in toluene/2,2-difluoroethan-1-ol (5 mL, 10/1, v/v) was added Cs₂CO₃ (626 mg, 1.92 mmol, 3.0 eq.), Pd(OAc)₂ (14 mg, 0.064 mmol, 0.1 eq.) and t-BuXPhos (54 mg, 0.13 mmol, 0.2 eq.), the reaction mixture was degassed with N₂ and stirred at 100° C. under N₂ atmosphere for 3 hrs. Then the reaction mixture was poured into H₂O (10 mL) and extracted with EtOAc (20 mL×3). The combined organic layers were washed with brine (20 mL), dried over Na₂SO₄ and concentrated under reduced pressure, the residue was purified by flash column chromatography on silica gel to give 6-(2,2-difluoroethoxy)-N-methoxy-2-((trans-4-methoxycyclohexyl)amino)-N-methylnicotinamide (170 mg, 71%) as a white solid. LC-MS (ESI): m/z 374 [M+H]⁺.

### Step B: 6-(2,2-difluoroethoxy)-2-((trans-4-methoxycyclohexyl)amino)nicotinaldehyde

To a solution of 6-(2,2-difluoroethoxy)-N-methoxy-2-((trans-4-methoxycyclohexyl)amino)-N-methylnicotinamide (170 mg, 0.455 mmol, 1.0 eq.) in THF (3 mL) was added LiAlH₄ (52 mg, 1.37 mmol, 3.0 eq.) at −65° C., the reaction mixture was stirred at −65° C. for 2 hrs. The reaction was quenched with NH₄Cl (sat. aq.) (5 mL), then allowed warm to room temperature, extracted with EtOAc (20 mL×3). The combined organic layers were washed with brine (10 mL), dried over Na₂SO₄ and concentrated under reduced pressure, the residue was purified by flash column chromatography on silica gel to give 6-(2,2-difluoroethoxy)-2-((trans-4-methoxycyclohexyl)amino)nicotinaldehyde (105 mg, 73%) as a colorless oil. LC-MS (ESI): m/z 315 [M+H]⁺.

### Step C: N-((6-(2,2-difluoroethoxy)-2-((trans-4-methoxycyclohexyl)amino)pyridin-3-yl)methyl)-2-methyl-2H-indazol-5-amine

To a solution of 6-(2,2-difluoroethoxy)-2-((trans-4-methoxycyclohexyl)amino)nicotinaldehyde (104 mg, 0.33 mmol, 1.0 eq.) and 2-methyl-2H-indazol-5-amine (58 mg, 0.4 mmol, 1.2 eq.) in DCE (3 mL) was added MgSO₄ (398 mg, 3.3 mmol, 10.0 eq.) and AcOH (79 μL, 1.32 mmol, 4.0 eq.), the reaction mixture was stirred at room temperature for 15 hrs. Then the reaction mixture was cooled to 0° C., NaBH(OAc)₃ (210 mg, 0.99 mmol, 3.0 eq.) was added in one portion, the resulting mixture was allowed warm to room temperature and stirred for 5 hrs. After completion, the reaction mixture was quenched with aqueous NaHCO₃ (sat. aq.) (10 mL) at 0° C., and extracted with EtOAc (15 mL×3), the combined organic layers were dried over Na₂SO₄ and concentrated under reduced pressure, the residue was purified by flash column chromatography on silica gel to give N-((6-(2,2-difluoroethoxy)-2-((trans-4-methoxycyclohexyl)amino)pyridin-3-yl)methyl)-2-methyl-2H-indazol-5-amine (70 mg, 47%) as a pale green solid. LC-MS (ESI): m/z 446 [M+H]⁺.

### Step D: 7-(2,2-difluoroethoxy)-1-(trans-4-methoxycyclohexyl)-3-(2-methyl-2H-indazol-5-yl)-3,4-dihydropyrido[2,3-d]pyrimidin-2(1H)-one

To a solution of N-((6-(2,2-difluoroethoxy)-2-((trans-4-methoxycyclohexyl)amino)pyridin-3-yl)methyl)-2-methyl-2H-indazol-5-amine (50 mg, 0.112 mmol, 1.0 eq.) and DIEA (58 mg, 0.45 mmol, 4.0 eq.) in THF (2 mL) was added triphosgene (13 mg, 45 μmol, 0.4 eq.) at 0° C., the reaction mixture was stirred at 50° C. for 3 hrs, then cooled to room temperature, t-BuOK (25 mg, 0.22 mmol, 2.0 eq.) was added and the reaction mixture was stirred at room temperature for additional 2 hrs. After completion, the reaction mixture was poured into ice water (10 mL) and extracted with EtOAc (15 mL×3), the combined organic layers were dried over Na₂SO₄ and concentrated under reduced pressure, the residue was purified by RP-Prep-HPLC to give 7-(2,2-difluoroethoxy)-1-(trans-4-methoxycyclohexyl)-3-(2-methyl-2H-indazol-5-yl)-3,4-dihydropyrido[2,3-d]pyrimidin-2(1H)-one (Example 429).

¹H NMR (400 MHz, CDCl₃) δ (ppm): 7.87 (s, 1H), 7.68 (d, J=9.2 Hz, 1H), 7.51 (s, 1H), 7.33 (d, J=8.0 Hz, 1H), 7.25-7.21 (m, 1H), 6.45 (d, J=7.6 Hz, 1H), 6.13 (t, J_{HF}=54.8 Hz, 1H), 4.66 (s, 2H), 4.64-4.47 (m, 3H), 4.22 (s, 3H), 3.36 (s, 3H), 3.25-3.15 (m, 1H), 2.63-2.54 (m, 2H), 2.23-2.16 (m, 2H), 1.90-1.85 (m, 2H), 1.40-1.31 (m, 2H).

LC-MS (ESI): m/z 472 [M+H]⁺.

### Synthesis of 5-amino-1-(4-(methoxy-d3)phenyl)-3-(2-methyl-2H-indazol-5-yl)-7-((2,2,2-trifluoroethyl)amino)-3,4-dihydropyrimido[4,5-d]pyrimidin-2(1H)-one (Example 430)

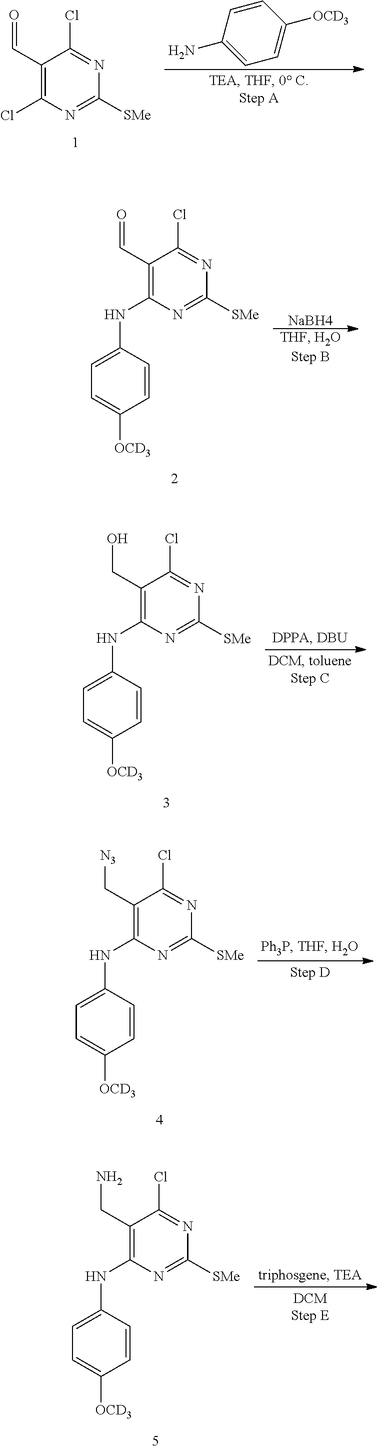

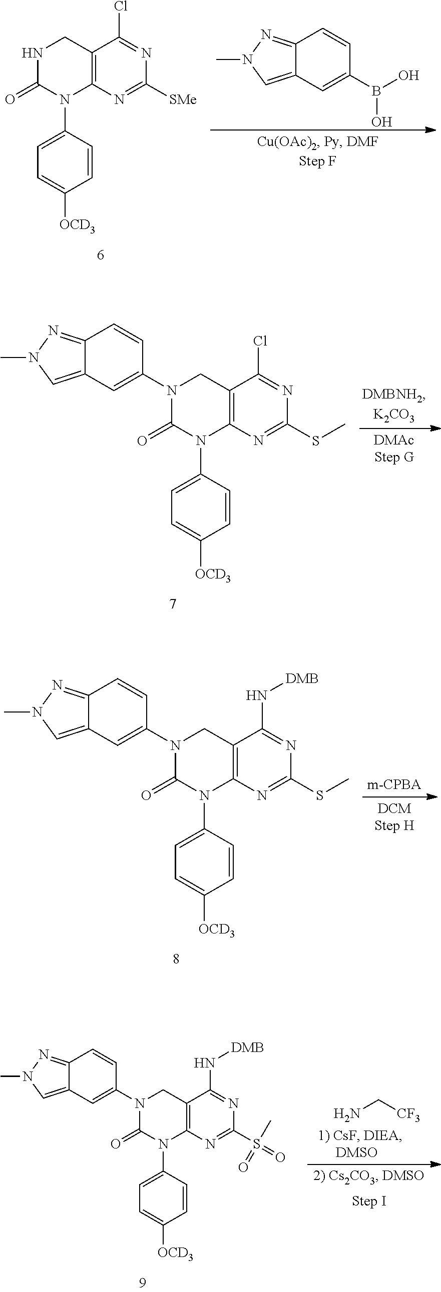

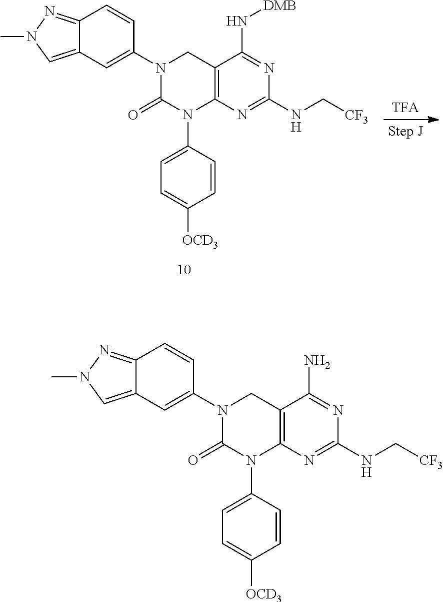

### Step A: 4-chloro-6-((4-(methoxy-d3)phenyl)amino)-2-(methylthio)pyrimidine-5-carbaldehyde

To a solution of 4-(methoxy-d3)aniline (2.5 g, 19.82 mmol, 1.0 eq.) in THF (70 mL) was added Et₃N (3.0 g, 30.0 mmol, 1.5 eq.), the reaction mixture was stirred at 0° C. for 10 min. Then 4,6-dichloro-2-(methylthio)pyrimidine-5-carbaldehyde (4.4 g, 19.82 mmol, 1.0 eq.) was added, the mixture was allowed to warm to room temperature and stirred for 2 hrs. The reaction mixture was concentrated under reduced pressure, the residue was purified by flash column chromatography on silica gel to give 4-chloro-6-((4-(methoxy-d₃)phenyl)amino)-2-(methylthio)pyrimidine-5-carbaldehyde (5.9 g, 97%) as a yellow solid.

LC-MS (ESI): m/z 313 [M+H]⁺.

¹H NMR (400 MHz, CDCl₃) δ (ppm): 11.05 (s, 1H), 10.23 (s, 1H), 7.49-7.43 (m, 2H), 6.87-6.79 (m, 2H), 2.44 (s, 3H).

### Step B: (4-chloro-6-((4-(methoxy-d3)phenyl)amino)-2-(methylthio)pyrimidin-5-yl)methanol

To a solution of 4-chloro-6-((4-(methoxy-d3)phenyl)amino)-2-(methylthio)pyrimidine-5-carbaldehyde (1.3 g, 4.16 mmol, 1.0 eq.) in THF (10 mL) and water (2 mL) was added NaBH₄ (0.24 g, 6.23 mmol, 1.0 eq.) at 0° C. Then the mixture was stirred at 20° C. for 4 hrs. The reaction mixture was quenched with water (40 mL) and extracted with EtOAc (40 mL×3), the combined organic layers were dried over anhydrous Na₂SO₄, filtered and concentrated under reduced pressure, the residue was purified by flash column chromatography on silica gel to give (4-chloro-6-((4-(methoxy-d3)phenyl)amino)-2-(methylthio)pyrimidin-5-yl)methanol (600 mg, 46%) as a white solid. LC-MS (ESI): m/z 315 [M+H]⁺.

### Step C: 5-(azidomethyl)-6-chloro-N-(4-(methoxy-d3)phenyl)-2-(methylthio)pyrimidin-4-amine

To a solution of (4-chloro-6-((4-(methoxy-d3)phenyl)amino)-2-(methylthio)pyrimidin-5-yl)methanol (720 mg, 2.29 mmol, 1.0 eq.) in DCM/toluene (1/1, v/v) (16 mL) was added DBU (696 mg, 4.57 mmol, 2.0 eq.), the reaction mixture was cooled to 0° C. and DPPA (1.1 g, 4.57 mmol, 2.0 eq.) was added, then the reaction mixture was stirred at room temperature for 15 hrs. After completion, the reaction mixture was diluted with H₂O (30 mL) and extracted with DCM (40 mL×3). The combined organic layers were washed with brine (30 mL), dried over Na₂SO₄ and concentrated under reduced pressure, the residue was purified by flash column chromatography on silica gel to give 5-(azidomethyl)-6-chloro-N-(4-(methoxy-d3)phenyl)-2-(methylthio)pyrimidin-4-amine (370 mg, 48%) as a pale yellow oil. LC-MS (ESI): m/z 340 [M+H]⁺.

### Step D: 5-(aminomethyl)-6-chloro-N-(4-(methoxy-d3)phenyl)-2-(methylthio)pyrimidin-4-amine

To a solution of 5-(azidomethyl)-6-chloro-N-(4-(methoxy-d3)phenyl)-2-(methylthio)pyrimidin-4-amine (370 mg, 1.09 mmol, 1.0 eq.) in THF (4 mL) was added H₂O (39 μL, 2.18 mmol, 2.0 eq) and PPh₃ (571 mg, 2.18 mmol, 2.0 eq), the reaction mixture was stirred at room temperature for 3 hrs. After completion, the reaction mixture was diluted with water (20 mL), extracted with EtOAc (40 mL×3). The combined organic layers were combined and washed with brine (20 mL), dried over Na₂SO₄ and concentrated under reduced pressure, the residue was purified by flash column chromatography on silica gel to give 5-(aminomethyl)-6-chloro-N-(4-(methoxy-d3)phenyl)-2-(methylthio)pyrimidin-4-amine (280 mg, 82%) as a pale yellow oil. LC-MS (ESI): m/z 314 [M+H]⁺.

### Step E: 5-chloro-1-(4-(methoxy-d3)phenyl)-7-(methylthio)-3,4-dihydropyrimido[4,5-d]pyrimidin-2(1H)-one

To a solution of 5-(aminomethyl)-6-chloro-N-(4-(methoxy-d3)phenyl)-2-(methylthio)pyrimidin-4-amine (280 mg, 0.89 mmol, 1.0 eq.) and Et₃N (361 mg, 3.57 mmol, 4.0 eq) in DCM (5 mL) was added triphosgene (132 mg, 0.45 mmol, 0.5 eq.) in one portion at 0° C., the reaction mixture was stirred at room temperature for 2 hrs. After completion, the reaction mixture was quenched by adding ice-cooled NaHCO₃ (sat. aq.) (30 mL), extracted with EtOAc (30 mL×3), the combined organic layers were combined and washed with brine (15 mL), dried over Na₂SO₄ and concentrated under reduced pressure, the residue was purified by flash column chromatography on silica gel to give 5-chloro-1-(4-(methoxy-d₃)phenyl)-7-(methylthio)-3,4-dihydropyrimido[4,5-d]pyrimidin-2(1H)-one (250 mg, 82%) as a white solid. LC-MS (ESI): m/z 340 [M+H]⁺.

### Step F: 5-chloro-1-(4-(methoxy-d₃)phenyl)-3-(2-methyl-2H-indazol-5-yl)-7-(methylthio)-3,4-dihydropyrimido[4,5-d]pyrimidin-2(1H)-one

To a solution of 5-chloro-1-(4-(methoxy-d₃)phenyl)-7-(methylthio)-3,4-dihydropyrimido[4,5-d]pyrimidin-2(1H)-one (500 mg, 1.47 mmol, 1.0 eq.) and (2-methyl-2H-indazol-5-yl)boronic acid (388 mg, 2.21 mmol, 1.5 eq.) in DMF (10 mL) was added Cu(OAc)₂ (267 mg, 1.47 mmol, 1.0 eq.) and pyridine (141 μL, 1.77 mmol, 1.2 eq.), the reaction mixture was stirred at 50° C. under air atmosphere for 24 hrs. The reaction mixture was filtered through a short pad of Celite®, the filtrate was diluted with H₂O (40 mL), extracted with EtOAc (20 mL×3), the combined organic layers were dried over Na₂SO₄, concentrated under reduced pressure, the residue was purified by flash column chromatography on silica gel to give 5-chloro-1-(4-(methoxy-d₃)phenyl)-3-(2-methyl-2H-indazol-5-yl)-7-(methylthio)-3,4-dihydropyrimido[4,5-d]pyrimidin-2(1H)-one (310 mg, 45%) as a white solid. LC-MS (ESI): m/z 470 [M+H]⁺.

### Step G: 5-((2,4-dimethoxybenzyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one

A mixture of 5-chloro-1-(4-(methoxy-d₃)phenyl)-3-(2-methyl-2H-indazol-5-yl)-7-(methylthio)-3,4-dihydropyrimido[4,5-d]pyrimidin-2(1H)-one (450 mg, 0.96 mmol, 1.0 eq.), (3,4-dimethoxyphenyl)methanamine (240 mg, 1.44 mmol, 1.5 eq.) and K₂CO₃ (397 mg, 2.87 mmol, 3.0 eq.) in DMAc (10 mL) was stirred at 80° C. for 2 hrs. TLC showed the reaction was completed. The reaction was diluted with water (30 ml), extracted with DCM (30 mL×3), the combined organic layers were washed with brine (20 mL), dried over anhydrous Na₂SO₄, filtered and concentrated under reduced pressure, the residue was purified by flash column chromatography on silica gel to give 5-((2,4-dimethoxybenzyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one (320 mg, 56%) as a white solid. LC-MS (ESI): m/z 601 [M+H]⁺.

### Step H: 5-((2,4-dimethoxybenzyl)amino)-1-(4-(methoxy-d₃)phenyl)-3-(2-methyl-2H-indazol-5-yl)-7-(methylsulfonyl)-3,4-dihydropyrimido[4,5-d]pyrimidin-2(1H)-one

To a solution of 5-((2,4-dimethoxybenzyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one (100 mg, 0.17 mmol, 1.0 eq.) in DCM (4 mL) was added mCPBA (103 mg, 0.51 mmol, 3.0 eq) in portions at 0° C., the reaction mixture was stirred at 0° C. for 2 hrs. After completion, the mixture was quenched with Na₂S₂O₃ (sat. aq.) (10 mL), extracted with DCM (20 mL×3). The combined organic layers were washed with brine (10 mL), dried over with anhydrous Na₂SO₄, filtered and concentrated under reduced pressure, the residue was purified by flash column chromatography on silica gel to give 5-((2,4-dimethoxybenzyl)amino)-1-(4-(methoxy-d₃)phenyl)-3-(2-methyl-2H-indazol-5-yl)-7-(methylsulfonyl)-3,4-dihydropyrimido[4,5-d]pyrimidin-2(1H)-one (90 mg, 84%) as an off-white solid. LC-MS (ESI): m/z 633 [M+H]⁺.

### Step I: 5-((2,4-dimethoxybenzyl)amino)-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one

A mixture of 5-((2,4-dimethoxybenzyl)amino)-1-(4-(methoxy-d₃)phenyl)-3-(2-methyl-2H-indazol-5-yl)-7-(methylsulfonyl)-3,4-dihydropyrimido[4,5-d]pyrimidin-2(1H)-one (90 mg, 0.14 mmol, 1.0 eq.), CsF (22 mg, 0.14 mmol, 1.0 eq.), DIEA (55 mg, 0.43 mmol, 3.0 eq.) and 2,2,2-trifluoroethan-1-amine (70 mg, 0.71 mmol, 5.0 eq.) in DMSO (6 mL) was stirred at 100° C. in a sealed tube for 12 hrs. Then Cs₂CO₃ (139 mg, 0.43 mmol, 3.0 eq.) was added to the mixture, then stirred at 100° C. for additional 12 hrs. The reaction mixture was diluted with water (20 mL), extracted with DCM (30 mL×3). The combined organic layers were washed with brine (10 mL), dried over with anhydrous Na₂SO₄, filtered and concentrated under reduced pressure, the residue was purified by flash column chromatography on silica gel to give 5-((2,4-dimethoxybenzyl)amino)-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one (70 mg, 77%) as a white solid. LC-MS (ESI): m/z 652 [M+H]⁺.

### Step J: 5-amino-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one

A solution of 5-((2,4-dimethoxybenzyl)amino)-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one (60 mg, 0.09 mmol, 1.0 eq.) in TFA (4 mL) was stirred at 50° C. for 15 hrs. The reaction mixture was concentrated under reduced pressure, the residue was diluted with DCM (30 mL), washed with NaHCO₃ (sat. aq.) (10 mL) and brine (10 mL), dried over anhydrous Na₂SO₄, filtered and concentrated in under reduced pressure, the residue was purified by RP-pre-HPLC to give 5-amino-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one (Example 430).

¹H NMR (400 MHz, DMSO-d₆) δ (ppm): 8.35 (s, 1H), 7.69 (s, 1H), 7.57 (d, J=9.2 Hz, 1H), 7.30-7.24 (m, 1H), 7.12 (d, J=8.8 Hz, 2H), 6.92 (d, J=8.8 Hz, 2H), 6.77 (s, 1H), 6.38 (s, 2H), 4.61 (s, 2H), 4.16 (s, 3H), 3.76 (s, 2H).

LC-MS (ESI): m/z 502 [M+H]⁺.

### Synthesis of 7-(2,2-difluoroethoxy)-3-(2-methoxy-1-methyl-1H-benzo[d]imidazol-6-yl)-1-(4-(methoxy-d₃)phenyl)-3,4-dihydropyrido[2,3-d]pyrimidin-2(1H)-one (Example 431)

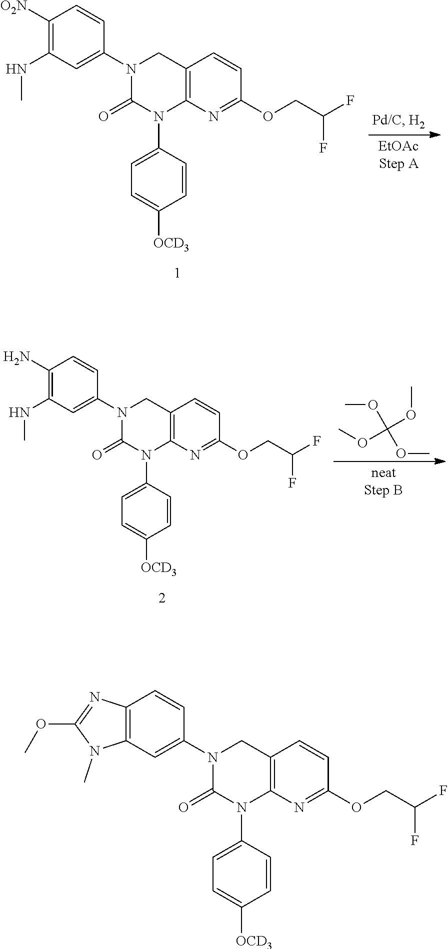

### Step A: 3-(4-amino-3-(methylamino)phenyl)-7-(2,2-difluoroethoxy)-1-(4-(methoxy-d₃)phenyl)-3,4-dihydropyrido[2,3-d]pyrimidin-2(1H)-one

To a solution of 7-(2,2-difluoroethoxy)-1-(4-(methoxy-d₃)phenyl)-3-(3-(methylamino)-4-nitrophenyl)-3,4-dihydropyrido[2,3-d]pyrimidin-2(1H)-one (170 mg, 0.35 mmol, 1.0 eq.) (synthesized from 5-bromo-N-methyl-2-nitroaniline &7-(2,2-difluoroethoxy)-1-(4-(methoxy-d₃)phenyl)-3,4-dihydropyrido[2,3-d]pyrimidin-2(1H)-one via Urea General Procedure I (Step E)) in EtOAc (8 mL) was added 10% Pd/C (20 mg), the reaction mixture was degassed with H₂, stirred at room temperature under H₂ atmosphere (1 atm) for 12 hrs. The reaction mixture was filtered through a short pad of Celite©, the filtrate was concentrated under reduced pressure to give 3-(4-amino-3-(methylamino)phenyl)-7-(2,2-difluoroethoxy)-1-(4-(methoxy-d₃)phenyl)-3,4-dihydropyrido[2,3-d]pyrimidin-2(1H)-one (110 mg, crude) as a brown oil which was used for next step directly without further purification. LC-MS (ESI): m/z 459 [M+H]⁺.

### Step B: 7-(2,2-difluoroethoxy)-3-(2-methoxy-1-methyl-TH-benzo[d]imidazol-6-yl)-1-(4-(methoxy-d₃)phenyl)-3,4-dihydropyrido[2,3-d]pyrimidin-2(1H)-one

A mixture of 3-(4-amino-3-(methylamino)phenyl)-7-(2,2-difluoroethoxy)-1-(4-(methoxy-d₃)phenyl)-3,4-dihydropyrido[2,3-d]pyrimidin-2(1H)-one (40 mg, 0.087 mmol, 1.0 eq.) and tetramethoxymethane (1 mL) was stirred at 100° C. in sealed tube under N₂ atmosphere for 12 hrs. the reaction mixture was poured into H₂O (20 mL) and extracted with DCM (15 mL×3). The combined organic layers were washed with brine (20 mL), dried over anhydrous Na₂SO₄ and concentrated under reduced pressure, the residue was purified by RP-Prep-HPLC to give 7-(2,2-difluoroethoxy)-3-(2-methoxy-1-methyl-TH-benzo[d]imidazol-6-yl)-1-(4-(methoxy-d₃)phenyl)-3,4-dihydropyrido[2,3-d]pyrimidin-2(1H)-one (Example 431).

¹H NMR (400 MHz, DMSO-d₆) δ (ppm): 7.65 (d, J=8.1 Hz, 1H), 7.43 (d, J=2.0 Hz, 1H), 7.40 (d, J=8.4 Hz, 1H), 7.29-7.19 (m, 2H), 7.13 (dd, J=8.4 Hz, 2.0 Hz, 1H), 7.05-6.95 (m, 2H), 6.52 (d, J=8.0 Hz, 1H), 6.00 (tt, J_{HF}=55.6 Hz, J=3.9 Hz, 1H), 4.90 (s, 2H), 4.10 (s, 3H), 4.05 (td, J_{HF}=14.5 Hz, J=3.9 Hz, 2H), 3.53 (s, 3H).

LC-MS (ESI): m/z 499 [M+H]⁺.

### Synthesis of 1-(4-(methoxy-d₃)phenyl)-3-(2-methyl-2H-indazol-5-yl)-5-(methylamino)-7-((2,2,2-trifluoroethyl)amino)-3,4-dihydropyrimido[4,5-d]pyrimidin-2(1H)-one (Example 432)

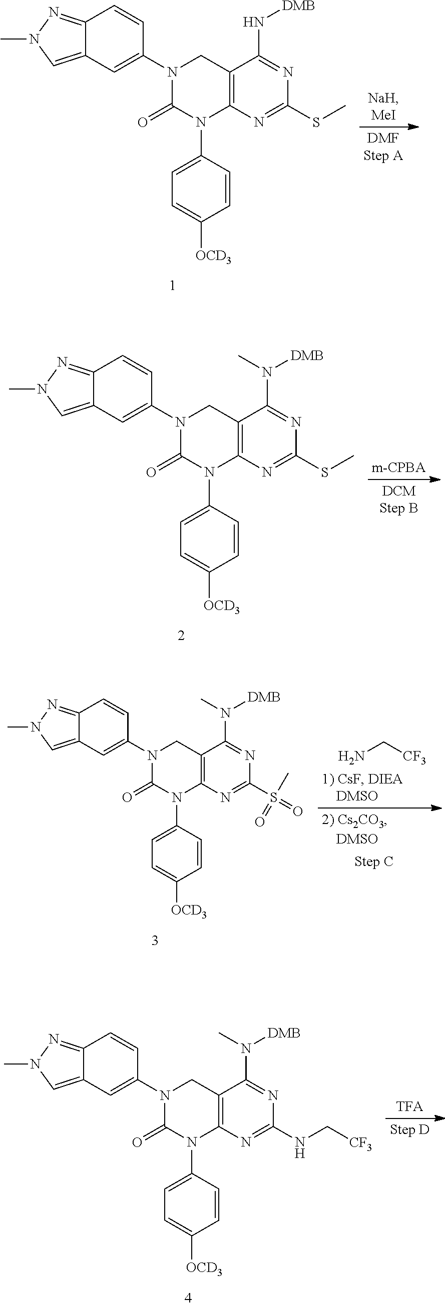

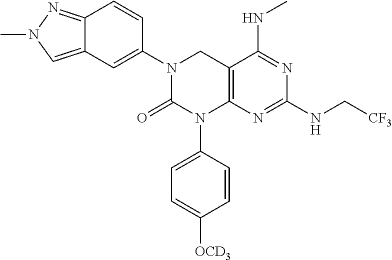

### Step A: 5-((2,4-dimethoxybenzyl)(methyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one

To a solution of 5-((2,4-dimethoxybenzyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one (200 mg, 0.33 mmol, 1.0 eq.) in DMF (6 mL) was added NaH (60% in mineral oil) (40 mg, 0.99 mmol, 3.0 eq.) in one portion at 0° C., the reaction mixture was stirred at 0° C. for additional 30 min. Then Mel (140 mg, 0.99 mmol, 3.0 eq.) was added via syringe, the resulting mixture was stirred at 0° C. for 2 hrs. After completion, the reaction was quenched with ice water (20 mL) and extracted with DCM (20 mL×3). The combined organic layers were washed with brine (20 mL), dried over Na₂SO₄ and concentrated under reduced pressure, the residue was purified by flash column chromatography on silica gel to give 5-((2,4-dimethoxybenzyl)(methyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one (170 mg, 83%) as an off-white solid. LC-MS (ESI): m/z 615 [M+H]⁺

### Step B: 5-((2,4-dimethoxybenzyl)(methyl)amino)-1-(4-(methoxy-d₃)phenyl)-3-(2-methyl-2H-indazol-5-yl)-7-(methylsulfonyl)-3,4-dihydropyrimido[4,5-d]pyrimidin-2(1H)-one

To a solution of 5-((2,4-dimethoxybenzyl)(methyl)amino)-1-(4-(methoxy-d₃)phenyl)-3-(2-methyl-2H-indazol-5-yl)-7-(methylthio)-3,4-dihydropyrimido[4,5-d]pyrimidin-2(1H)-one (170 mg, 0.28 mmol, 1.0 eq.) in DCM (4 mL) was added mCPBA (168 mg, 0.83 mmol, 3.0 eq.) in several portions at 0° C., the reaction mixture was stirred at 0° C. for 2 hrs. The reaction was quenched with Na₂S₂O₃ (sat. aq.) (10 mL), extracted with DCM (10 mL×3). The combined organic layers were washed with brine (30 mL), dried over Na₂SO₄ and concentrated under reduced pressure, the residue was purified by flash column chromatography on silica gel to give 5-((2,4-dimethoxybenzyl)(methyl)amino)-1-(4-(methoxy-d₃)phenyl)-3-(2-methyl-2H-indazol-5-yl)-7-(methylsulfonyl)-3,4-dihydropyrimido[4,5-d]pyrimidin-2(1H)-one (12 Omg, 66%) as an off-white solid. LC-MS (ESI): m/z 647 [M+H]⁺.

### Step C: 5-((2,4-dimethoxybenzyl)(methyl)amino)-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one

A mixture of 5-((2,4-dimethoxybenzyl)(methyl)amino)-1-(4-(methoxy-d₃)phenyl)-3-(2-methyl-2H-indazol-5-yl)-7-(methylsulfonyl)-3,4-dihydropyrimido[4,5-d]pyrimidin-2(1H)-one (120 mg, 0.18 mmol, 1.0 eq.), CsF (28 mg, 0.18 mmol, 1.0 eq.), DIEA (71 mg, 0.54 mmol, 3.0 eq.) and 2,2,2-trifluoroethan-1-amine (89 mg, 0.90 mmol, 5.0 eq.) in DMSO (6 mL) was stirred at 100° C. in a sealed tube for 12 hrs. Then Cs₂CO₃ (176 mg, 0.54 mmol, 3.0 eq.) was added, the resulting mixture was stirred at 100° C. for additional 12 hrs. The reaction mixture was diluted with water (20 mL), extracted with DCM (10 mL×3). The combined organic layers were washed with brine (20 mL), dried over with anhydrous Na₂SO₄, filtered and concentrated under reduced pressure, the residue was purified by flash column chromatography on silica gel to give 5-((2,4-dimethoxybenzyl)(methyl)amino)-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one (80 mg, 67%) as a white solid. LC-MS (ESI): m/z 666 [M+H]⁺.

### Step D: 1-(4-(methoxy-d₃)phenyl)-3-(2-methyl-2H-indazol-5-yl)-5-(methylamino)-7-((2,2,2-trifluoroethyl)amino)-3,4-dihydropyrimido[4,5-d]pyrimidin-2(1H)-one

A solution of 5-((2,4-dimethoxybenzyl)(methyl)amino)-1-(4-(methoxy-d₃)phenyl)-3-(2-methyl-2H-indazol-5-yl)-7-((2,2,2-trifluoroethyl)amino)-3,4-dihydropyrimido[4,5-d]pyrimidin-2(1H)-one (80 mg, 0.12 mmol, 1.0 eq.) in TFA (4 mL) was stirred at 50° C. for 15 hrs. The reaction mixture was concentrated under reduced pressure, the residue was diluted with DCM (20 mL), washed with NaHCO₃ (sat. aq.) (10 mL) and brine (10 mL), dried over anhydrous Na₂SO₄, filtered and concentrated in under reduced pressure, the residue was purified by RP-prep-HPLC to give 1-(4-(methoxy-d₃)phenyl)-3-(2-methyl-2H-indazol-5-yl)-5-(methylamino)-7-((2,2,2-trifluoroethyl)amino)-3,4-dihydropyrimido[4,5-d]pyrimidin-2(1H)-one (Example 432).

¹H NMR (400 MHz, DMSO-d₆) δ (ppm): 8.30 (s, 1H), 7.67 (s, 1H), 7.56 (d, J=9.2 Hz, 1H), 7.24 (dd, J=9.2 Hz, 2.0 Hz, 1H), 7.08 (d, J=8.9 Hz, 2H), 6.91 (d, J=8.9 Hz, 2H), 4.56 (s, 2H), 4.13 (s, 3H), 3.43 (s, 2H), 2.77 (s, 3H).

LC-MS (ESI): m/z 516.1 [M+H]⁺.

Synthesis of 3-(2-methyl-2H-indazol-5-yl)-1-(6-methylpyridin-3-yl)-7-((2,2,2-trifluoroethyl)amino)-3,4-dihydropyrimido[4,5-d]pyrimidin-2(1H)-one (Example 433)

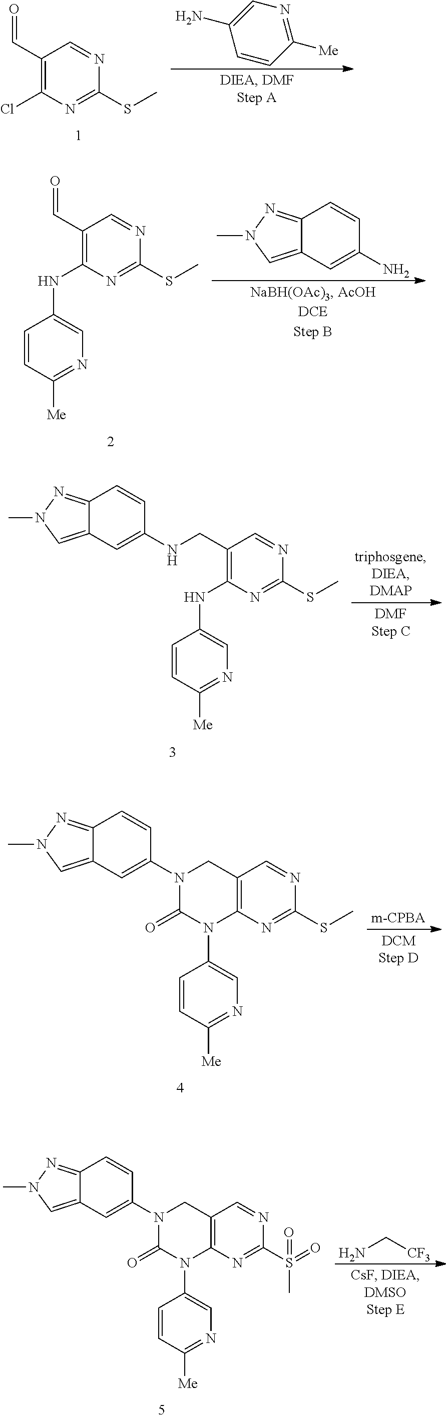

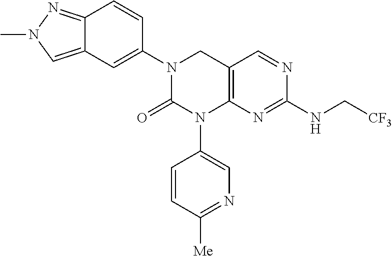 [SMILES: Cc1ccc(N2C(=O)N(c3ccc4nn(C)cc4c3)Cc3cnc(NCC(F)(F)F)nc32)cn1]

### Step A: 4-((6-methylpyridin-3-yl)amino)-2-(methylthio)pyrimidine-5-carbaldehyde

To a solution of 4-chloro-2-(methylsulfanyl)pyrimidine-5-carbaldehyde (1.0 g, 5.3 mmol, 1.0 eq.) in DMF (10 mL) was added DIEA (1.02 g, 8.0 mmol, 1.5 eq.) and 6-methylpyridin-3-amine (573 mg, 5.3 mmol, 1.0 eq.), the reaction mixture was stirred at 0° C. for 20 min, then it was allowed to warm to room temperature and stirred additional 14 hrs. After the completion, the reaction mixture was diluted with H₂O (50 mL), extracted with EtOAc (20 mL×3). The combined organic layers were washed with brine (30 mL) and dried over Na₂SO₄, concentrated under reduced pressure, the residue was purified by flash chromatography to afford 4-[(6-methylpyridin-3-yl)amino]-2-(methylsulfanyl)pyrimidine-5-carbaldehyde (1.21 g, 88%) as a white solid. LC-MS (ESI): m/z 261 [M+H]⁺.

### Step B: 2-methyl-N-((4-((6-methylpyridin-3-yl)amino)-2-(methylthio)pyrimidin-5-yl)methyl)-2H-indazol-5-amine

To a solution of 4-2-(methylsulfanyl)pyrimidine-5-carbaldehyde (750 mg, 2.9 mmol, 1.0 eq.) and 2-methyl-2H-indazol-5-amine (466 mg, 3.2 mmol 1.1 eq.) in DCE/MeOH (6 mL, 1/1, v/v) was added HOAc (0.5 mL, 8.7 mmol, 3.0 eq.) at 0° C. The mixture was stirred at room temperature for 30 min, then NaBH₃CN (908 mg, 14.4 mmol, 5.0 eq.) was added at 0° C. in several portions, after addition, the reaction mixture was allowed to warm to room temperature, and stirred at the same temperature for additional 1 hr. After the completion, the reaction mixture was quenched by adding NaHCO₃ (sat. aq.) (20 mL), extracted with EtOAc (30 mL×3), the combined organic layers were washed with brine (50 mL), dried over anhydrous Na₂SO₄ and concentrated under reduced pressure, the residue was purified by flash chromatography to afford 2-methyl-N-({4-2-(methylsulfanyl)pyrimidin-5-yl}methyl)-2H-indazol-5-amine (1.01 g, 90%) as a white solid. LC-MS (ESI): m/z 392 [M+H]⁺.

### Step C: 3-(2-methyl-2H-indazol-5-yl)-1-(6-methylpyridin-3-yl)-7-(methylthio)-3,4-dihydropyrimido[4,5-d]pyrimidin-2(1H)-one

To a solution of 2-methyl-N-({4-2-(methylsulfanyl)pyrimidin-5-yl}methyl)-2H-indazol-5-amine (550 mg, 1.4 mmol, 1.0 eq.) in 1,4-dioxane (5 mL) was added triphosgene (250 mg, 0.84 mmol, 0.6 eq.), DIPEA (725 mg, 5.6 mmol, 4.0 eq.) and DMAP (17 mg, 0.14 mmol, 0.1 eq.), the resulting mixture was stirred at 80° C. for 4 h. After the completion, the pH was adjusted to ˜6 by adding 2N HCl (aq.), then diluted with H₂O (50 mL), extracted with EtOAc (20 mL×3), the combined organic layers were washed with brine (30 mL), dried over anhydrous Na₂SO₄ and concentrated under reduced pressure, the residue was purified by flash chromatography to afford 3-(2-methyl-2H-indazol-5-yl)-1-(6-methylpyridin-3-yl)-7-(methylsulfanyl)-1H,2H,3H,4H-diazinopyrimidin-2-one (446 mg, 76%) as a white solid. LC-MS (ESI): m/z 418 [M+H]⁺.

### Step D: 3-(2-methyl-2H-indazol-5-yl)-1-(6-methylpyridin-3-yl)-7-(methylsulfonyl)-3,4-dihydropyrimido[4,5-d]pyrimidin-2(1H)-one

To a solution of 3-(2-methyl-2H-indazol-5-yl)-1-(6-methylpyridin-3-yl)-7-(methylsulfanyl)-1H,2H,3H,4H-diazinopyrimidin-2-one (100 mg, 0.24 mmol, 1.0 eq.) in DCM (1 mL) was added mCPBA (97 mg, 0.48 mmol, 2.0 eq.) in one portion, the reaction mixture was stirred at room temperature for 1 hr, after the completion, the reaction was quenched with Na₂S₂O₄ (sat. aq.) to destroy the excess oxidant, extracted with EtOAc (10 mL×3), the combined organic layers were washed with brine (20 mL), dried over anhydrous Na₂SO₄ and concentrated under reduced pressure, the residue was purified by flash chromatography on silica gel to afford 7-methanesulfonyl-3-(2-methyl-2H-indazol-5-yl)-1-(6-methylpyridin-3-yl)-1H,2H,3H,4H-diazinopyrimidin-2-one (106 mg, 98%) as a white solid. LC-MS (ESI): m/z 450 [M+H]⁺.

### Step E: 3-(2-methyl-2H-indazol-5-yl)-1-(6-methylpyridin-3-yl)-7-((2,2,2-trifluoroethyl)amino)-3,4-dihydropyrimido[4,5-d]pyrimidin-2(1H)-one (Example 433)

To a solution of 7-methanesulfonyl-3-(2-methyl-2H-indazol-5-yl)-1-(6-methylpyridin-3-yl)-1H,2H,3H,4H-diazinopyrimidin-2-one (108 mg, 0.24 mmol, 1.0 eq.) in DMSO (1 mL) was added DIEA (155 mg, 1.2 mmol, 5.0 eq.), 2,2,2-trifluoroethan-1-amine (119 mg, 1.2 mmol, 5 eq.), CsF (37 mg, 0.24 mmol, 1.0 eq.), the resulting mixture was stirred at 100° C. for 14 hrs. After the completion, the reaction mixture was diluted with H₂O (10 mL), extracted with EtOAc (10 mL×3), the combined organic layers were washed with brine (20 mL) and dried over Na₂SO₄ and concentrated under reduced pressure, the residue was purified by RP-prep-HPLC to afford 3-(2-methyl-2H-indazol-5-yl)-1-(6-methylpyridin-3-yl)-7-1H,2H,3H,4H-diazinopyrimidin-2-one. (Example 433)

¹H NMR (400 MHz, DMSO-d₆) δ (ppm): δ: 8.37 (d, J=2.4 Hz, 1H), 8.36 (s, 1H), 8.15 (s, 1H), 7.71 (d, J=1.4 Hz, 1H), 7.66 (dd, J=8.1 Hz, 2.4 Hz, 1H), 7.59 (d, J=9.2 Hz, 1H), 7.50 (br, 1H) 7.35 (d, J=8.2 Hz, 1H), 7.28 (dd, J=9.2 Hz, 2.0 Hz, 1H), 4.85 (s, 2H), 4.17 (s, 3H), 3.82 (br, 2H), 2.51 (s, 3H).

LC-MS (ESI): m/z 469 [M+H]⁺.

### Synthesis of 3-(1,2-dimethyl-1H-benzo[d]imidazol-6-yl)-7-isopropoxy-1-(4-(methoxy-d₃)phenyl)-3,4-dihydropyrido[2,3-d]pyrimidin-2(1H)-one (Example 434)

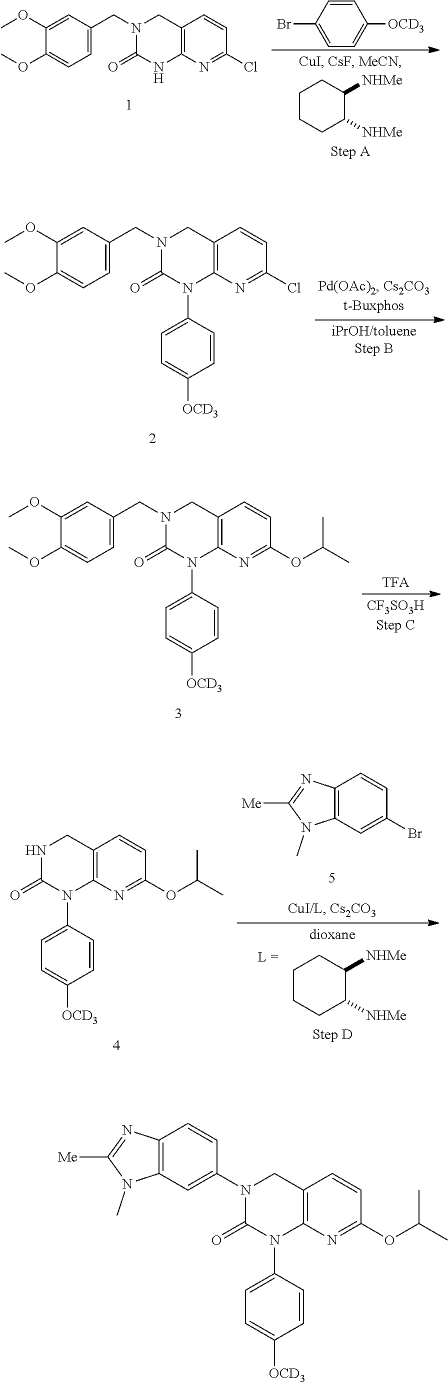

### Step A: 7-chloro-3-(3,4-dimethoxybenzyl)-1-(4-(methoxy-d3)phenyl)-3,4-dihydropyrido[2,3-d]pyrimidin-2(1H)-one

A mixture of 7-chloro-3-(3,4-dimethoxybenzyl)-3,4-dihydropyrido[2,3-d]pyrimidin-2(1H)-one (300 mg, 0.9 mmol, 1.0 eq.), 1-bromo-4-(methoxy-d₃)benzene (256 mg, 1.4 mmol, 1.5 eq.), CuI (171 mg, 0.9 mmol, 1.0 eq.), (1R,2R)—N1,N2-dimethylcyclohexane-1,2-diamine (256 mg, 1.8 mmol, 2.0 eq.), CsF (410 mg, 2.7 mmol, 3.0 eq.) and ACN (4 mL) was stirred at 85° C. under N₂ atmosphere for 14 hrs. After the completion, the reaction mixture was concentrated under reduced pressure, the residue was purified by silica gel column to afford 7-chloro-3-(3,4-dimethoxybenzyl)-1-(4-(methoxy-d₃)phenyl)-3,4-dihydropyrido[2,3-d]pyrimidin-2(1H)-one (200 mg, 50%) as a brown solid.

LC-MS (ESI): m/z 443 [M+H]⁺.

### Step B: 3-(3,4-dimethoxybenzyl)-7-isopropoxy-1-(4-(methoxy-d3)phenyl)-3,4-dihydropyrido[2,3-d]pyrimidin-2(1H)-one

To a solution of 7-chloro-3-(3,4-dimethoxybenzyl)-1-(4-(methoxy-d₃)phenyl)-3,4-dihydropyrido[2,3-d]pyrimidin-2(1H)-one (150 mg, 0.34 mmol, 1.0 eq.), Pd(OAc)₂ (7.6 mg, 0.03 mmol, 0.1 eq.), t-BuXPhos (28.8 mg, 0.07 mmol, 0.2 eq.) in i-PrOH (4 mL) and toluene (1 mL) was added Cs₂CO₃ (331 mg, 1 mmol, 3.0 eq.). The mixture was stirred at 80° C. under N₂ atmosphere for 14 hrs, After the completion, the reaction mixture was concentrated under reduced pressure, the residue was purified by flash column to give 3-(3,4-dimethoxybenzyl)-7-isopropoxy-1-(4-(methoxy-d₃)phenyl)-3,4-dihydropyrido[2,3-d]pyrimidin-2(1H)-one (45 mg, 29%) as a white solid. LC-MS (ESI): m/z 467 [M+H]⁺.

### Step C: 7-isopropoxy-1-(4-(methoxy-d₃)phenyl)-3,4-dihydropyrido[2,3-d]pyrimidin-2(1H)-one

To a solution of 3-(3,4-dimethoxybenzyl)-7-isopropoxy-1-(4-(methoxy-d₃)phenyl)-3,4-dihydropyrido[2,3-d]pyrimidin-2(1H)-one (50 mg, 0.1 mmol, 1.0 eq.) in TFA (4 mL) was added CF₃SO₃H (47 □L, 0.5 mmol, 5.0 eq.) slowly. The mixture was stirred at room temperature for 3 hrs. The reaction was quenched by adding ice-cooled NaHCO₃ (sat. aq.) (10 mL), then extracted with EtOAc (10 mL×3), the combined organic layers was washed with brine (20 mL), dried over Na₂SO₄ and concentrated under reduced pressure, the residue was purified by flash column chromatography on silica gel to give 7-isopropoxy-1-(4-(methoxy-d₃)phenyl)-3,4-dihydropyrido[2,3-d]pyrimidin-2(1H)-one (30 mg, 88%) as a white solid. LC-MS (ESI): m/z 317 [M+H]⁺.

### Step D: 3-(1,2-dimethyl-1H-benzo[d]imidazol-6-yl)-7-isopropoxy-1-(4-(methoxy-d₃)phenyl)-3,4-dihydropyrido[2,3-d]pyrimidin-2(1H)-one (Example 434)

A mixture of compound 7-isopropoxy-1-(4-(methoxy-d₃)phenyl)-3,4-dihydropyrido[2,3-d]pyrimidin-2(1H)-one (30 mg, 0.1 mmol, 1.0 eq.), 6-bromo-1,2-dimethyl-1H-benzo[d]imidazole (32 mg, 0.14 mmol, 1.5 eq.), CuI (19 mg, 0.1 mmol, 1.0 eq.), (1R,2R)—N₁,N₂-dimethylcyclohexane-1,2-diamine (27 mg, 0.2 mmol, 2.0 eq.), Cs₂CO₃ (93 mg, 0.3 mmol, 3.0 eq.) and dioxane (1.5 mL) were added to a sealed tube, the resulting mixture was irradiated under microwave (150 W) at 120° C. for 1.5 hrs. The reaction mixture was concentrated under reduced pressure, the residue was purified by RP-prep-HPLC to give 3-(1,2-dimethyl-1H-benzo[d]imidazol-6-yl)-7-isopropoxy-1-(4-(methoxy-d₃)phenyl)-3,4-dihydropyrido[2,3-d]pyrimidin-2(1H)-one (Example 434).

1H NMR (400 MHz, DMSO-d₆) δ: 7.57 (d, J=6.4 Hz, 1H), 7.55 (s, 1H), 7.50 (d, J=8.8 Hz, 1H), 7.22 (d, J=9.2 Hz, 2H), 7.17 (dd, J=8.8 Hz, 2.0 Hz, 1H), 6.98 (d, J=9.2 Hz, 2H), 6.34 (d, J=8.0 Hz, 1H), 4.89 (s, 2H), 4.55-4.49 (m, 1H), 3.72 (s, 3H), 2.52 (s, 3H), 1.05 (d, J=6.4 Hz, 6H).

LC-MS (ESI): m/z 461 [M+H]⁺.

### Synthesis of 3-(1,2-dimethyl-1H-benzo[d]imidazol-6-yl)-1-(4-(methoxy-d3)phenyl)-5-methyl-7-((2,2,2-trifluoroethyl)amino)-3,4-dihydropyrimido[4,5-d]pyrimidin-2(1H)-one (Example 435)

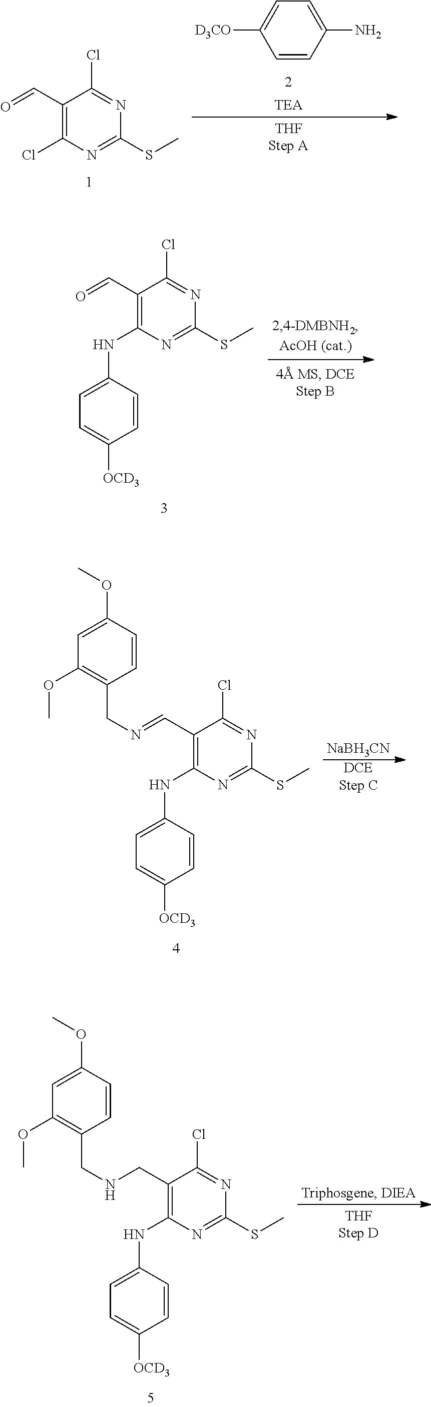

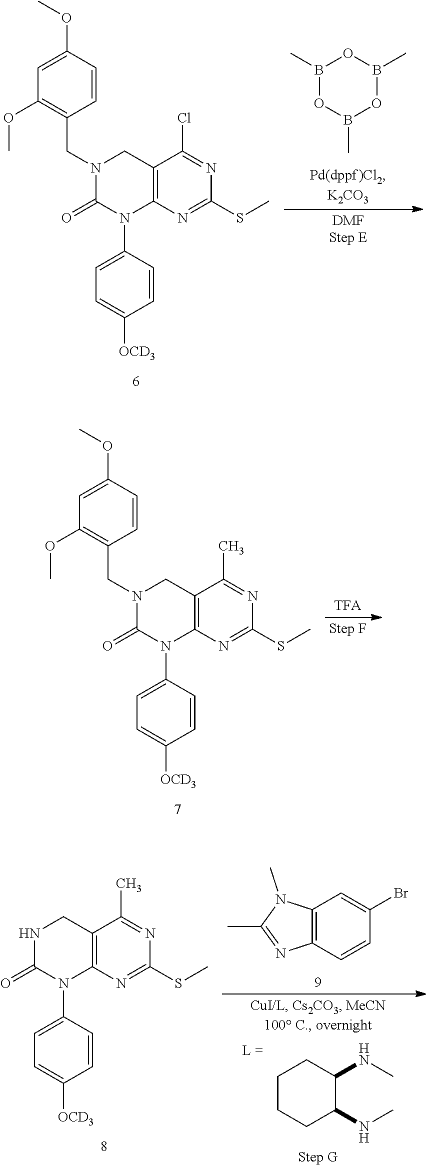

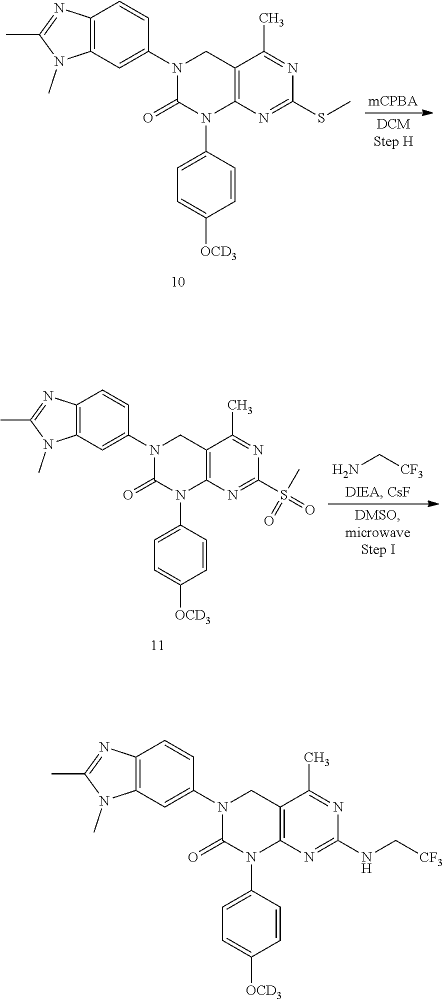

Step A: 4-chloro-6-((4-(methoxy-d₃)phenyl)amino)-2-(methylthio)pyrimidine-5-carbaldehyde 4-(methoxy-d₃)aniline (2.5 g, 19.82 mmol, 1.0 eq.) in dry THF (30 mL) was added Et₃N (4.12 mL, 29.73 mmol, 1.5 eq), the resulting mixture was cooled down to 0° C. with an ice-water bath and stirred for additional 30 min, then 4,6-dichloro-2-(methylthio)pyrimidine-5-carbaldehyde (4.4 g, 19.82 mmol, 1.0 eq.) in dry THF (10 mL) was added at 0° C. drop-wisely, then allowed to warm to room temperature and stirred for additional 2 hrs. After the completion, The reaction was quenched by adding H₂O (30 mL), extracted with EtOAc (20 mL×3), the combined organic layers were washed with dilute HCl (0.5 N, aq.) (10 mL), brine (20 mL), dried over anhydrous Na₂SO₄ and concentrated under reduced pressure, the residue was purified by flash column chromatography on silica gel to give 4-chloro-6-((4-(methoxy-d₃)phenyl)amino)-2-(methylthio)pyrimidine-5-carbaldehyde (5.9 g, 97%) as a yellow solid. LC-MS (ESI): m/z 313 [M+H]⁺

### Step B: (E)-6-chloro-5-(((2,4-dimethoxybenzyl)imino)methyl)-N-(4-(methoxy-d₃)phenyl)-2-(methylthio)pyrimidin-4-amine

4-chloro-6-((4-(methoxy-d₃)phenyl)amino)-2-(methylthio)pyrimidine-5-carbaldehyde (6.6 g, 21.2 mmol, 1.0 eq.) in dry DCE (100 ml), was added 2,4-dimethoxyphenyl)methanamine (3.34 mL, 22.3 mmol, 1.05 eq.), ground 4A molecular sieve (5 g) and several drops of AcOH, the resulting mixture was stirred at room temperature for 14 hrs. After the completion, the molecular sieve was removed by filtration, the filtrate was concentrated under reduced pressure to give the crude (E)-6-chloro-5-(((2,4-dimethoxybenzyl)imino)methyl)-N-(4-(methoxy-d₃)phenyl)-2-(methylthio)pyrimidin-4-amine (10 g, crude) as a yellow thick oil, which used in next step without further purification. LC-MS (ESI): m/z 462 [M+H]⁺.

Step C: 6-chloro-5-(((2,4-dimethoxybenzyl)amino)methyl)-N-(4-(methoxy-d₃)phenyl)-2-(methylthio)pyrimidin-4-amine (E)-6-chloro-5-(((2,4-dimethoxybenzyl)imino)methyl)-N-(4-(methoxy-d₃)phenyl)-2-(methylthio)pyrimidin-4-amine (8.0 g, 17.35 mmol, 1.0 eq.) was dissolved in dry DCE (100 mL) and cooled down to 0° C. with an ice-water bath, then NaBH₃CN (5.47 g, 86.8 mmol, 5.0 eq.) was added in several portions during 30 min, after the addition, the reaction mixture was allowed to warm to room temperature, and stirred for additional 3 hrs. After the completion, the reaction mixture was quenched carefully by adding ice water (100 mL), extracted with DCM (100 mL×5). The combined organic layers were washed with brine (200 mL), dried over anhydrous Na₂SO₄ and concentrated under reduced pressure, the residue was purified by flash column chromatography on silica gel to give 6-chloro-5-(((2,4-dimethoxybenzyl)amino)methyl)-N-(4-(methoxy-d₃)phenyl)-2-(methylthio)pyrimidin-4-amine (5.2 g, 65%) as a yellow solid. LC-MS (ESI): m/z 464 [M+H]⁺.

### Step D: 5-chloro-3-(2,4-dimethoxybenzyl)-1-(4-(methoxy-d₃)phenyl)-7-(methylthio)-3,4-dihydropyrimido[4,5-d]pyrimidin-2(1H)-one

6-chloro-5-(((2,4-dimethoxybenzyl)amino)methyl)-N-(4-(methoxy-d3)phenyl)-2-(methylthio)pyrimidin-4-amine (5.2 g, 11.2 mmol, 1.0 eq.) and DIEA (7.224 g, 56.0 mmol, 5.0 eq.) were dissolved in dry THF (50 mL), the mixture was cooled down to 0° C. with an ice-water bath, then triphosgene (1.99 g, 6.7 mmol, 0.6 eq.) in THF (5 mL) was added drop-wisely via a syringe. After the addition, the reaction mixture was allowed to warm to room temperature and stirred for additional 1 hr. After completion, the reaction was quenched by adding NaHCO₃ (sat. aq.) (50 mL), extracted with EtOAc (50 mL×3), the combined organic layers were washed with brine (50 mL), dried over anhydrous Na₂SO₄ and concentrated under reduced pressure, and the residue was purified by flash column chromatography on silica gel to give 5-chloro-3-(2,4-dimethoxybenzyl)-1-(4-(methoxy-d₃)phenyl)-7-(methylthio)-3,4-dihydropyrimido[4,5-d]pyrimidin-2(1H)-one (4.2 g, 76%) as a white solid. LC-MS (ESI): m/z 490 [M+H]+.

### Step E: 3-(2,4-dimethoxybenzyl)-1-(4-(methoxy-d₃)phenyl)-5-methyl-7-(methylthio)-3,4-dihydropyrimido[4,5-d]pyrimidin-2(1H)-one

To a mixture of 5-chloro-3-(2,4-dimethoxybenzyl)-1-(4-(methoxy-d₃)phenyl)-7-(methylthio)-3,4-dihydropyrimido[4,5-d]pyrimidin-2(1H)-one (1.5 g, 3.07 mmol, 1.0 eq.), 2,4,6-trimethyl-1,3,5,2,4,6-trioxatriborinane (385 mg, 3.07 mmol, 1.0 eq.) and K₂CO₃ (1.3 g, 9.21 mmol, 3.0 eq.) in DMF (20 mL), was added Pd(dppf)Cl₂ (449 mg, 0.62 mmol, 0.2 eq.), the resulting mixture was stirred at 100° C. under N₂ atmosphere for 14 hrs. After the completion, the reaction mixture was filtered through a short pad of Celite®, the filtrate was concentrated under reduced pressure, the residue was purified by flash column chromatography on silica gel to give 3-(2,4-dimethoxybenzyl)-1-(4-(methoxy-d₃)phenyl)-5-methyl-7-(methylthio)-3,4-dihydropyrimido[4,5-d]pyrimidin-2(1H)-one (670 mg, 47%) as a yellow solid. LC-MS (ESI): m/z 470 [M+H]⁺.

### Step F: 1-(4-(methoxy-d3)phenyl)-5-methyl-7-(methylthio)-3,4-dihydropyrimido[4,5-d]pyrimidin-2(1H)-one

3-(2,4-dimethoxybenzyl)-1-(4-(methoxy-d3)phenyl)-5-methyl-7-(methylthio)-3,4-dihydropyrimido[4,5-d]pyrimidin-2(1H)-one (670 mg, 1.43 mmol, 1.0 eq.) was dissolved in dry TFA (5 mL), the resulting mixture was stirred at room temperature for 3 hrs. The reaction was concentrated under reduced pressure to remove excess of TFA, and the residue was re-dissolved in EtOAc (10 mL), NaHCO₃ (sat. aq) was added until aqueous layer was pH=8, then extracted with EtOAc (10 mL×3), the combined organic layers were washed with brine (20 mL), dried over anhydrous Na₂SO₄ and concentrated under reduced pressure, the residue was purified by flash column chromatography on silica gel to give 1-(4-(methoxy-d₃)phenyl)-5-methyl-7-(methylthio)-3,4-dihydropyrimido[4,5-d]pyrimidin-2(1H)-one (400 mg, 88%) as a yellow solid. LC-MS (ESI): m/z 320 [M+H]⁺.

### Step G: 3-(1,2-dimethyl-1H-benzo[d]imidazol-6-yl)-1-(4-(methoxy-d₃)phenyl)-5-methyl-7-(methylthio)-3,4-dihydropyrimido[4,5-d]pyrimidin-2(1H)-one

A mixture of 1-(4-(methoxy-d₃)phenyl)-5-methyl-7-(methylthio)-3,4-dihydropyrimido[4,5-d]pyrimidin-2(1H)-one (400 mg, 1.25 mmol, 1.0 eq.), 6-bromo-1,2-dimethyl-1H-benzo[d]imidazole (Ref. Bioorg. Med. Chem., 2016, 24, 2486-2503) (395 mg, 1.88 mmol, 1.5 eq.), CuI (239 mg, 1.25 mmol, 1.0 eq.), Cs₂CO₃ (1.23 mg, 3.75 mmol, 3.0 eq.) and trans-N₁,N₂-dimethylcyclohexane-1,2-diamine (357 mg, 2.508 mmol, 2.0 eq.) was suspended in dry ACN (10 mL), the resulting mixture was stirred at 100° C. for 14 hrs. After the completion, the reaction was quenched by adding H₂O (20 mL), and extracted with EtOAc (20 mL×3). The combined organic layers were washed with brine (10 mL), dried over anhydrous Na₂SO₄ and concentrated under reduced pressure, the residue was purified by flash column chromatography on silica gel to give 3-(1,2-dimethyl-1H-benzo[d]imidazol-6-yl)-1-(4-(methoxy-d₃)phenyl)-5-methyl-7-(methylthio)-3,4-dihydropyrimido[4,5-d]pyrimidin-2(1H)-one (250 mg, 43%) as a yellow solid. LC-MS (ESI): m/z 464 [M+H]⁺.

### Step H: 3-(1,2-dimethyl-1H-benzo[d]imidazol-6-yl)-1-(4-(methoxy-d3)phenyl)-5-methyl-7-(methylsulfonyl)-3,4-dihydropyrimido[4,5-d]pyrimidin-2(1H)-one

3-(1,2-dimethyl-1H-benzo[d]imidazol-6-yl)-1-(4-(methoxy-d₃)phenyl)-5-methyl-7-(methylthio)-3,4-dihydropyrimido[4,5-d]pyrimidin-2(1H)-one (250 mg, 0.54 mmol, 1.0 eq.) was dissolved in dry DCM (10 mL) and cooled down to 0° C. with an ice-water bath, then mCPBA (70% wt) (280 mg, 1.62 mmol, 3.0 eq.) was added in several portions, after the addition, the mixture was allowed to warm to room temperature and stirred for another 14 hrs. After the completion, the excess of mCPBA was quenched by adding NaHSO₃ sat. aq.) (until KI-starch test paper was no longer blue), then extracted with EtOAc (10 mL×3). The combined organic layers were washed with brine (10 mL), dried over anhydrous Na₂SO₄ and concentrated under reduced pressure, the residue was purified by flash column chromatography on silica gel to give 3-(1,2-dimethyl-1H-benzo[d]imidazol-6-yl)-1-(4-(methoxy-d3)phenyl)-5-methyl-7-(methylsulfonyl)-3,4-dihydropyrimido[4,5-d]pyrimidin-2(1H)-one (100 mg, 37%) as a white solid. LC-MS (ESI): m/z 496 [M+H]⁺.

### Step I: 3-(1,2-dimethyl-1H-benzo[d]imidazol-6-yl)-1-(4-(methoxy-d3)phenyl)-5-methyl-7-((2,2,2-trifluoroethyl)amino)-3,4-dihydropyrimido[4,5-d]pyrimidin-2(1H)-one (Example 435)

3-(1,2-dimethyl-1H-benzo[d]imidazol-6-yl)-1-(4-(methoxy-d3)phenyl)-5-methyl-7-(methylsulfonyl)-3,4-dihydropyrimido[4,5-d]pyrimidin-2(1H)-one (100 mg, 0.202 mmol, 1.0 eq.), DIEA (166 μL, 1.01 mmol, 5.0 eq.), CsF (30 mg, 0.202 mmol, 1.0 eq.) and 2,2,2-trifluoroethan-1-amine (80 μL, 1.01 mmol, 5.0 eq.) were dissolved in DMSO (5 mL), the resulting mixture was under microwave irradiation (150 W) at 100° C. for 2 hrs. After the completion, the reaction mixture was concentrated under reduced pressure, the residue was purified by RP-prep-HPLC to give 3-(1,2-dimethyl-1H-benzo[d]imidazol-6-yl)-1-(4-(methoxy-d3)phenyl)-5-methyl-7-((2,2,2-trifluoroethyl)amino)-3,4-dihydropyrimido[4,5-d]pyrimidin-2(1H)-one (Example 435)

¹H NMR (400 MHz, DMSO-d₆) δ 8.36 (s, 1H), 7.57 (d, J=1.6 Hz, 1H), 7.51 (d, J=8.4 Hz, 1H), 7.20 (dd, J=8.8 Hz, 2.0 Hz, 1H), 7.16 (d, J=8.8 Hz, 2H), 6.96 (d, J=8.8 Hz, 2H), 4.82 (s, 2H), 3.88-3.79 (m, 2H), 3.72 (s, 3H), 2.53 (s, 3H), 2.20 (s, 3H).

LC-MS (ESI): m/z 515 [M+H]⁺.

### General Procedure X

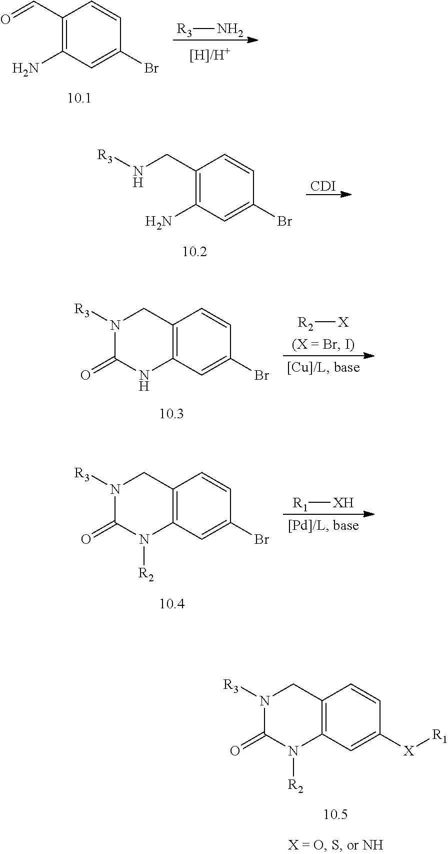

Compounds of structure 10.5 may be obtained through the scheme depicted as General Procedure X. Beginning with starting aldehyde 10.1, a reductive amination may be performed to introduce the desired R₃ group. The resulting diamine 10.2 may then be reacted with CDI to form the cyclic urea 10.3. The desired R₂ group may then be introduced through a copper mediated C—N coupling reaction to afford 10.4. Lastly, the desired R₁ group may be installed with a palladium mediated C—X coupling reaction to afford compounds of structure 10.5.

### Preparation of Example 287 Via General Procedure X

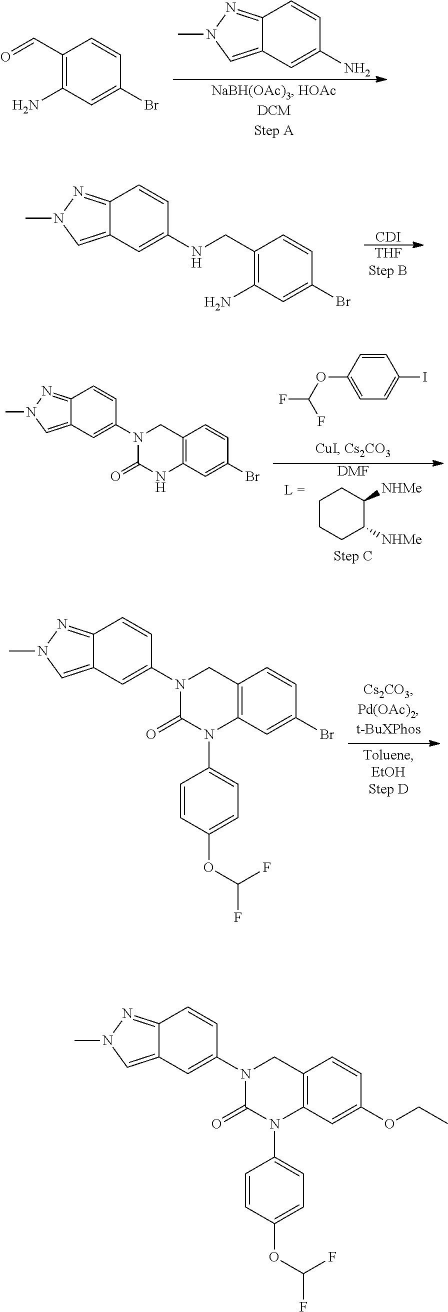

### Step A: N-(2-amino-4-bromobenzyl)-2-methyl-2H-indazol-5-amine

To a mixture of 2-amino-4-bromobenzaldehyde (500 mg, 2.5 mmol, 1.0 eq.) and 2-methyl-2H-indazol-5-amine (368 mg, 2.5 mmol, 1.0 eq.) in DCM (15 mL) was added AcOH (600 mg, 10 mmol, 4.0 eq.) at 0° C. The reaction mixture was stirred at room temperature for 3 hrs. Then the reaction mixture was cooled to 0° C., NaBH(OAc)₃ (1657 mg, 7.5 mmol, 3.0 eq.) was added in several portions during 30 min, after addition, the reaction mixture was allowed to warm to room temperature and stirred for 5 hrs. The reaction was quenched by adding NaHCO₃ (sat. aq.) (15 mL) at 0° C., extracted with EtOAc (20 mL×3). The combined organic layers were dried over Na₂SO₄, concentrated under reduced pressure, purified by silica gel column chromatography to afford N-(2-amino-4-bromobenzyl)-2-methyl-2H-indazol-5-amine (650 mg, 78.5% yield) as a brown solid. LC-MS (ESI): m/z=331, 333 [M+H]⁺.

### Step B: 7-bromo-3-(2-methyl-2H-indazol-5-yl)-3,4-dihydroquinazolin-2(1H)-one

To a solution of N-(2-amino-4-bromobenzyl)-2-methyl-2H-indazol-5-amine (650 mg, 1.9 mmol, 1.0 eq.) in THF (15 mL) was added CDI (477 mg, 2.9 mmol, 1.5 eq.), the reaction mixture was stirred at 80° C. for 8 hrs. The progress of the reaction was monitored by LC-MS (ESI), after completion, the reaction was quenched with ice water (15 mL) and extracted with EtOAc (20 mL×3), The combined organic layers were washed with brine (20 mL) and dried over Na₂SO₄, concentrated under reduced pressure, the residue was purified by flash column chromatography on silica gel to afford 7-bromo-3-(2-methyl-2H-indazol-5-yl)-3,4-dihydroquinazolin-2(1H)-one (450 mg, 66% yield) as a brown solid. LC-MS (ESI): m/z=357, 359 [M+H]⁺.

### Step C: 7-bromo-1-(4-(difluoromethoxy)phenyl)-3-(2-methyl-2H-indazol-5-yl)-3,4-dihydroquinazolin-2(1H)-one

To a solution of 7-bromo-3-(2-methyl-2H-indazol-5-yl)-1,2,3,4-tetrahydroquinazolin-2-one (150 mg, 0.42 mmol, 1.0 eq.) in DMF (5 mL) was added 1-(difluoromethoxy)-4-iodobenzene (340 mg, 1.26 mmol, 3.0 eq.), Cs₂CO₃ (410 mg, 1.26 mmol, 3.0 eq.), CuI (80 mg, 0.42 mmol, 1.0 eq.) and N¹,N²-dimethylcyclohexane-1,2-diamine (0.13 mL, 0.84 mmol, 2.0 eq.), the reaction mixture was stirred under N₂ atmosphere at 100° C. for 24 hrs. The progress of the reaction was monitored by LC-MS (ESI), after completion, the reaction was diluted with water (15 mL) and extracted with EtOAc (20 mL×3), The combined organic layers were washed with brine (20 mL) and dried over Na₂SO₄, concentrated under reduced pressure, the crude residue was purified by flash column chromatography on silica gel to afford 7-bromo-1-[4-(difluoromethoxy)phenyl]-3-(2-methyl-2H-indazol-5-yl)-1,2,3,4-tetrahydroquinazolin-2-one (65 mg, 31% yield) as a yellow oil. LC-MS (ESI): m/z=499, 501 [M+H]⁺.

### Step D: 1-[4-(difluoromethoxy)phenyl]-7-ethoxy-3-(2-methyl-2H-indazol-5-yl)-1,2,3,4-tetrahydroquinazolin-2-one

To a solution of 7-bromo-1-[4-(difluoromethoxy)phenyl]-3-(2-methyl-2H-indazol-5-yl)-1,2,3,4-tetrahydroquinazolin-2-one (55 mg, 0.11 mmol, 1.0 eq.) in toluene (7 mL) and EtOH (0.7 mL) was added Cs₂CO₃ (108 mg, 0.33 mmol, 3.0 eq.), Pd(OAc)₂ (3 mg, 0.011 mmol, 0.1 eq.) and t-BuXPhos (9 mg, 0.022 mmol, 0.2 eq.), the reaction mixture was stirred under N₂ atmosphere at 100° C. for 12 hrs. The progress of the reaction was monitored by LC-MS (ESI), after completion, the reaction was diluted with water (10 mL) and extracted with EtOAc (20 mL×3), The combined organic layers were washed with brine (20 mL) and dried over Na₂S₄, concentrated under reduced pressure, the crude residue was purified by Prep-HPLC to afford 1-[4-(difluoromethoxy)phenyl]-7-ethoxy-3-(2-methyl-2H-indazol-5-yl)-1,2,3,4-tetrahydroquinazolin-2-one (Example 287). LC-MS (ESI): m/z 465.1 [M+H]⁺

¹H NMR (400 MHz, CDCl₃) δ (ppm): 7.96 (s, 1H), 7.76 (d, J 9.2 Hz, 1H), 7.66 (s, 1H), 7.48 (d, J=8.8 Hz, 1H), 7.38 (d, J=8.4 Hz, 2H), 7.25 (d, J=8.4 Hz, 2H), 7.06 (d, J 8.4 Hz, 1H), 6.57 (dd, J 9.6 Hz, 2.0 Hz, 1H), 6.56 (t, J=74.0 Hz, 1H), 5.89 (d, J=2.0 Hz, 1H), 4.91 (s, 2H), 4.34 (s, 3H), 3.89 (q, J=7.2 Hz, 2H), 1.33 (t, J=7.2 Hz, 3H).

The procedure set forth above for General Procedure X was used to synthesize the following compounds by using appropriate starting materials:

| Cpd No. | Structure | Characterization |
|---|---|---|
| Example 288 | 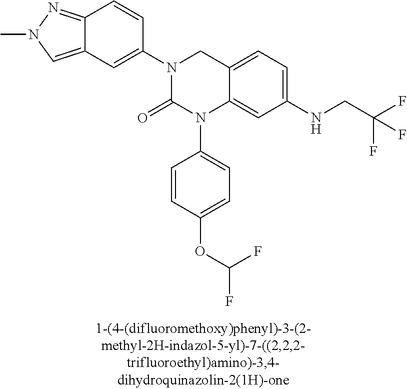 [SMILES: Cn1cc2cc(N3Cc4ccc(NCC(F)(F)F)cc4N(c4ccc(OC(F)F)cc4)C3=O)ccc2n1] | LC-MS (ESI): m/z 518.1 [M + H]+. 1H NMR (400 MHz, Chloroform- d) δ 7.86 (s, 1H), 7.68 (d, J = 9.2 Hz, 1H), 7.59 (s, 1H), 7.39 (d, J = 8.3 Hz, 2H), 7.31 (d, J = 9.2 Hz, 1H), 7.24 (d, J = 8.3 Hz, 2H), 6.99 (d, J = 8.1 Hz, 1H), 6.56 (t, J = 73.6 Hz, 1H), 6.35 (d, J = 8.1 Hz, 1H), 5.62 (s, 1H), 4.87 (s, 2H), 4.21 (s, 3H), 3.87 (t, J = 6.8 Hz, 1H), 3.67 3-58 (m, 2H). |
| Example 436 | 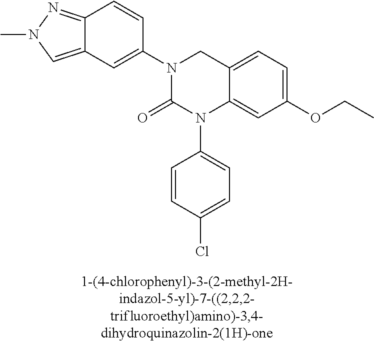 | LC-MS: m/z 486 (M + H)+. 1H NMR (400 MHz, DMSO-d₆) δ: 8.33 (s, 1H), 7.64 (s, 1H), 7.59 (d, J = 8.4 Hz, 2H), 7.54 (d, J = 9.1 Hz, 1H), 7.40 (d, J = 8.5 Hz, 2H), 7.26 (d, J = 7.7 Hz, 1H), 7.03 (d, J = 8.4 Hz, 1H), 6.40 (d, J = 10.0 Hz, 1H), 6.23 (t, J = 7.2 Hz, 1H), 5.60 (d, J = 2.0 Hz, 1H), 4.84 (s, 2H), 4.17 (s, 3H), 3.81-3.72 (m, 2H). |
| Example 437 | 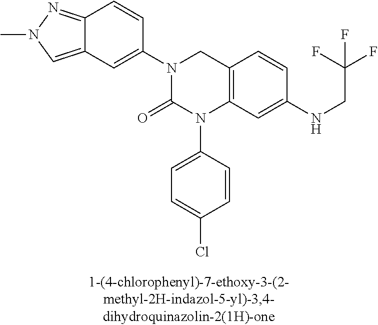 | LC-MS: m/z 433 (M + H)+. 1H NMR (400 MHz, DMSO-d₆) δ: 8.33 (s, 1H), 7.66 (s, 1H), 7.60 (d, J = 8.4 Hz, 2H), 7.55 (d, J = 9.2 Hz, 1H), 7.42 (d, J = 8.0 Hz, 2H), 7.26 (d, J = 9.6 Hz, 1H), 7.20 (d, J = 8.4 Hz, 1H), 6.61 (d, J = 8.0 Hz, 1H), 5.62 (s, 1H), 4.91 (s, 2H), 4.16 (s, 3H), 3.85 (q, J = 6.8 Hz, 2H), 1.22 (t, J = 6.8 Hz, 3H). |

The table below provides a list of prophetic compounds of Formula II that may be synthesized using the General Procedure X shown above, or a combination of other procedures described herein using ordinary skill.

1-(4-(1H-1,2,4-triazol-3-yl)phenyl)-3-(2-methylbenzo[d]thiazol-6-yl)-7-
((2,2,2-trifluoroethyl)amino)-3,4-dihydroquinazolin-2(1H)-one
1-(4-(difluoromethoxy)phenyl)-3-(1-methyl-1H-benzo[d]imidazol-6-yl)-7-
((2,2,2-trifluoroethyl)amino)-3,4-dihydroquinazolin-2(1H)-one
1-(4-(difluoromethoxy)phenyl)-7-(ethylamino)-3-(1-methyl-1H-
benzo[d]imidazol-6-yl)-3,4-dihydroquinazolin-2(1H)-one
1-(4-(1H-1,2,4-triazol-3-yl)phenyl)-3-(2-methyl-2H-indazol-5-yl)-7-
((2,2,2-trifluoroethyl)amino)-3,4-d]hydroquinazolin-2(1H)-one
1-(4-(1H-1,2,4-triazol-3-yl)phenyl)-3-(4-methoxyphenyl)-7-
((2,2,2-trifluoroethyl)amino)-3,4-d]hydroquinazolin-2(1H)-one
1-(4-(4H-1,2,4-triazol-3-yl)phenyl)-7-ethoxy-3-(2-methyl-2H-
indazol-5-yl)-3,4-dihydroquinazolin-2(1H)-one
7-ethoxy-1-(6-(fluoromethyl)pyridin-3-yl)-3-(2-methyl-2H-
indazol-5-yl)-3,4-dihydroquinazolin-2(1H)-one
7-(2,2-difluoroethoxy)-1-(4-(difluoromethoxy)phenyl)-3-(2-methyl-2H-
indazol-5-yl)-3,4-dihydroquinazolin-2(1H)-one
7-(2,2-difluoroethoxy)-1-(4-methoxyphenyl)-3-(2-methyl-2H-
indazol-5-yl)-3,4-dihydroquinazolin-2(1H)-one
7-(2,2-difluoroethoxy)-1-(4-(difluoromethoxy)phenyl)-3-(1-methyl-1H-
benzo[d]imidazol-6-yl)-3,4-dihydroquinazolin-2(1H)-one
7-(2,2-difluoroethoxy)-1-(4-methoxyphenyl)-3-(1-methyl-1H-
benzo[d]imidazol-6-yl)-3,4-dihydroquinazolin-2(1H)-one
7-ethoxy-1-(4-(methoxy-d3)phenyl)-3-
(2-methyl-2H-indazol-5-yl)-3,4-dihydroquinazolin-2(1H)-one
7-(2,2-difluoroethoxy)-1-(4-(methoxy-d3)phenyl)-3-(2-methyl-2H-
indazol-5-yl)-3,4-dihydroquinazolin-2(1H)-one
7-(2,2-difluoroethoxy)-1-(4-(methoxy-d3)phenyl)-3-(1-methyl-1H-
benzo[d]imidazol-6-yl)-3,4-dihydroquinazolin-2(1H)-one
7-(2,2-difluoroethoxy)-3-(2,3-dimethyl-2H-indazol-5-yl)-1-(4-
(methoxy-d3)phenyl)-3,4-dihydroquinazolin-2(1H)-one
7-(2,2-difluoroethoxy)-3-(3-methoxy-2-methyl-2H-indazol-5-yl)-1-(4-
(methoxy-d3)phenyl)-3,4-dihydroquinazolin-2(1H)-one
7-(2,2-difluoroethoxy)-3-(3-(methoxy-d3)-2-methyl-2H-
indazol-5-yl)-1-(4-(methoxy-d3)phenyl)-3,4-dihydroquinazolin-2(1H)-one
1-(4-bromophenyl)-7-(2,2-difluoroethoxy)-3-(2-methyl-2H-
indazol-5-yl)-3,4-dihydroquinazolin-2(1H)-one
1-(4-methoxyphenyl)-3-(2-methyl-2H-indazol-5-yl)-7-
propyl-3,4-dihydroquinazolin-2(1H)-one
7-isopropoxy-1-(4-(methoxy-d3)phenyl)-3-(2-methyl-2H-
indazol-5-yl)-3,4-dihydroquinazolin-2(1H)-one
7-(2,2-difluoroethoxy)-3-(2-ethyl-2H-indazol-5-yl)-1-(4-
(methoxy-d3)phenyl)-3,4-dihydroquinazolin-2(1H)-one
7-(2,2-difluoroethoxy)-1-(4-(methoxy-d3)phenyl)-3-(2-(2-methoxyethyl)-
2H-indazol-5-yl)-3,4-dihydroquinazolin-2(1H)-one
7-(2,2-difluoroethoxy)-1-(1H-indol-5-yl)-3-(2-methyl-2H-
indazol-5-yl)-3,4-dihydroquinazolin-2(1H)-one
7-(2,2-difluoroethoxy)-1-(4-(methoxy-d3)phenyl)-5-methyl-3-(2-methyl-
2H-indazol-5-yl)-3,4-dihydroquinazolin-2(1H)-one
1-(4-methoxyphenyl)-5-methyl-3-(2-methyl-2H-indazol-5-yl)-7-
((2,2,2-trifluoroethyl)amino)-3,4-dihydroquinazolin-2(1H)-one
1-(benzo[d]thiazol-5-yl)-7-(2,2-difluoroethoxy)-3-(2-methyl-2H-
indazol-5-yl)-3,4-dihydroquinazolin-2(1H)-one
7-(2,2-difluoroethoxy)-1-(1-methyl-1H-indol-5-yl)-3-(2-methyl-2H-
indazol-5-yl)-3,4-dihydroquinazolin-2(1H)-one
7-((1,1-difluoropropan-2-yl)oxy)-1-(4-(methoxy-d3)phenyl)-3-
(2-methyl-2H-indazol-5-yl)-3,4-dihydroquinazolin-2(1H)-one

### General Procedure XI

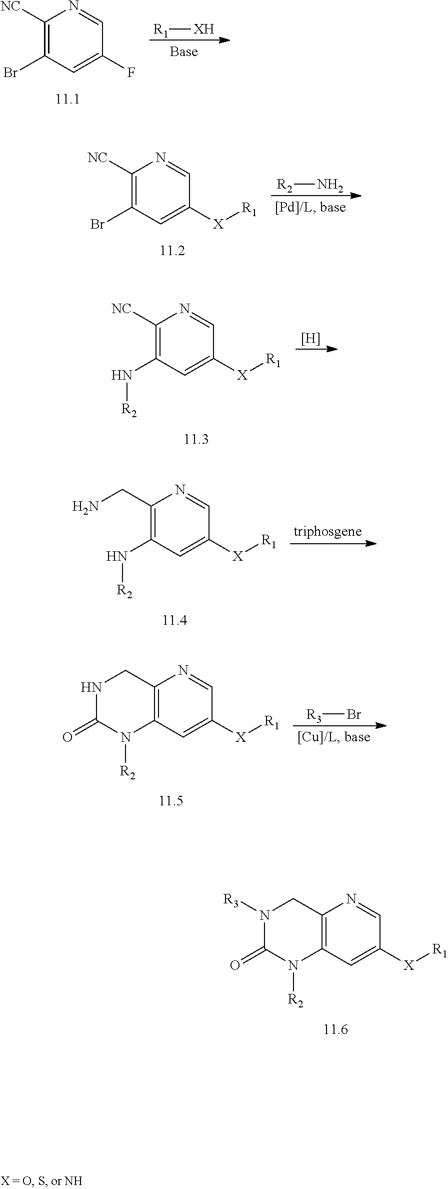

Compounds of structure 11.6 may be obtained through the scheme depicted as General Procedure XI. Beginning with aryl fluoride 11.1, the desired R₁ group may be introduced with a base mediated aromatic substitution to generate compound 11.2. The desired R₂ group may then be introduced through a palladium mediated C—N coupling reaction to generate aryl amine 11.3. Nitrile 11.3 may then be reduced with a hydride source to generate diamine 11.4. Diamine 11.4 may then be cyclized using triphosgene to generate cyclic urea 11.5. Lastly, the desired R₃ group may be introduced with a copper mediated C—N coupling reaction to afford compounds of structure 11.5.

### Preparation of Example 289 Via General Procedure XI

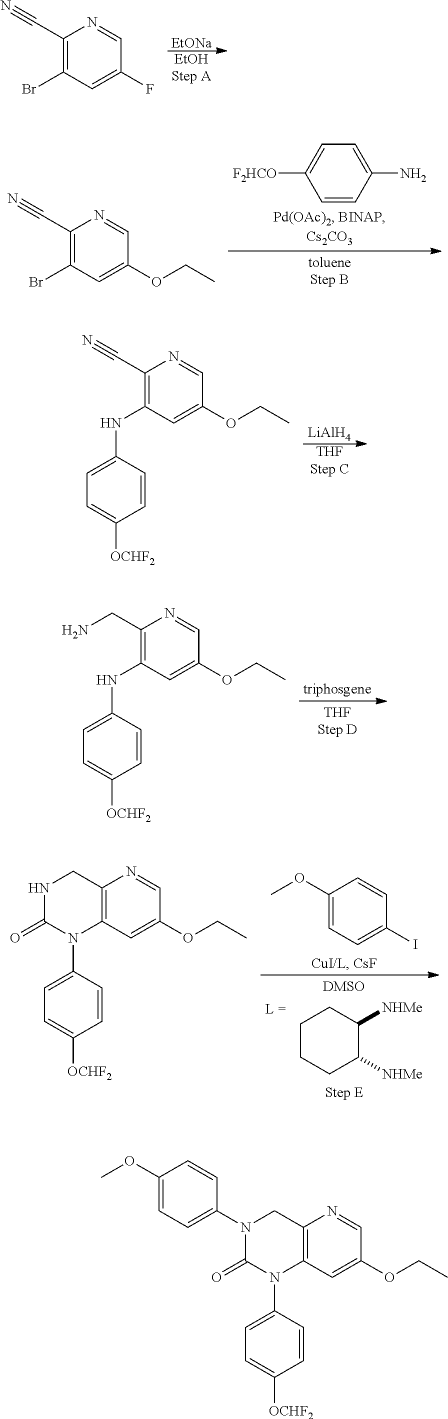

### Step A: 3-bromo-5-ethoxypicolinonitrile

To a solution of 3-bromo-5-fluoropyridine-2-carbonitrile (1.0 g, 4.9 mmol, 1.0 eq.) in EtOH (5 mL) was added EtONa (339 mg, 4.9 mmol, 1.0 eq.) in portions, then the reaction mixture was stirred at room temperature for 3 hrs. The progress of the reaction was monitored by LC-MS (ESI), after completion, the reaction mixture was cooled to room temperature and concentrated under reduced pressure, the residue was diluted with water (30 mL), extracted with DCM (20 mL×2). The combined organic layers were washed with brine (20 mL), dried over Na₂SO₄ and concentrated under reduced pressure, the residue was purified by flash column chromatography on silica gel to afford 3-bromo-5-ethoxypyridine-2-carbonitrile (620 mg, 55% yield) as a yellow solid. LC-MS (ESI): m/z 228 [M+H]⁺

### Step B: 3-((4-(difluoromethoxy)phenyl)amino)-5-ethoxypicolinonitrile

To a solution of 3-bromo-5-ethoxypyridine-2-carbonitrile (200 mg, 0.88 mmol, 1.0 eq.) in toluene (5 mL) was added Cs₂CO₃ (867 mg, 2.64 mmol, 3.0 eq.), BINAP (55 mg, 0.09 mmol, 0.1 eq.), Pd(OAc)₂ (20 mg, 0.09 mmol, 0.1 eq.) and 4-(difluoromethoxy)aniline (140 mg, 0.88 mmol, 0.1 eq.), the reaction mixture was stirred 90° C. under N₂ atmosphere for 15 hrs. The reaction mixture was diluted with H₂O (20 ml), extracted with EtOAc (20 mL×3), the combined organic layers were washed with brine (30 ml), dried over with Na₂SO₄ and concentrated under reduced pressure, the residue was purified by flash column chromatography on silica gel to afford 3-((4-(difluoromethoxy)phenyl)amino)-5-ethoxypicolinonitrile (122 mg, 45% yield) as a solid. LC-MS (ESI): m/z 306 [M+H]⁺.

### Step C: 2-(aminomethyl)-N-(4-(difluoromethoxy)phenyl)-5-ethoxypyridin-3-amine

To a suspension of LiAlH₄ (115 mg, 3.02 mmol, 2.0 eq.) in THF (4 mL) was added a solution of 3-((4-(difluoromethoxy)phenyl)amino)-5-ethoxypicolinonitrile (462 mg, 1.51 mmol, 1.0 eq.) in THF (1 mL) dropwise at 0° C., the reaction mixture was stirred at 0° C. for 0.5 hr. The progress of the reaction was monitored by TLC, after completion, the reaction was diluted with dry THF (10 mL) and quenched with water (0.12 mL), aq. NaOH (0.12 mL, 15% wt) and water (0.36 mL) in sequence, then anhydrous Na₂SO₄ (5 g) was added, the resulting mixture was stirred vigorously for additional 30 min, filtered through a short pad of Celite®, the filtrate was concentrated under reduced pressure, the residue was purified by flash column chromatography on silica gel to afford 2-(aminomethyl)-N-(4-(difluoromethoxy)phenyl)-5-ethoxypyridin-3-amine (272 mg, 58% yield). LC-MS (ESI): m/z 310 [M+H]⁺.

### Step D: 1-(4-(difluoromethoxy)phenyl)-7-ethoxy-3,4-dihydropyrido[3,2-d]pyrimidin-2(1H)-one

To a solution of 2-(aminomethyl)-N-(4-(difluoromethoxy)phenyl)-5-ethoxypyridin-3-amine (100 mg, 0.31 mmol, 1.0 eq.) in THF (4 mL) was added triphosgene (78 mg, 0.26 mmol, 2.0 eq.), the reaction mixture was stirred at room temperature for 4 hrs The progress of the reaction was monitored by LC-MS (ESI), after completion, the reaction was quenched with water (15 mL) and extracted with ethyl acetate (20 mL×3). The combined organic layers were washed with brine (30 mL), dried over Na₂SO₄, and concentrated under reduced pressure, the residue was purified by flash column chromatography on silica gel to afford 1-(4-(difluoromethoxy)phenyl)-7-ethoxy-3,4-dihydropyrido[3,2-d]pyrimidin-2(1H)-one (80 mg, 74% yield) as a white solid. LC-MS (ESI): m/z 336 [M+H]⁺.

### Step E: 1-(4-(difluoromethoxy)phenyl)-7-ethoxy-3-(4-methoxyphenyl)-3,4-dihydropyrido[3,2-d]pyrimidin-2(1H)-one

A mixture of 1-(4-(difluoromethoxy)phenyl)-7-ethoxy-3,4-dihydropyrido[3,2-d]pyrimidin-2(1H)-one (40 mg, 0.12 mmol, 1.0 eq.), 1-iodo-4-methoxybenzene (42 mg, 0.18 mmol, 1.5 eq.), CuI (23 mg, 0.12 mmol, 1.0 eq.), N¹,N²-dimethylcyclohexane-1,2-diamine (34 mg, 0.24 mmol, 2.0 eq.) and CsF (54 mg, 0.36 mmol, 3.0 eq.) in DMSO (2 mL) stirred under N₂ atmosphere at 100° C. for 16 hrs. The reaction mixture was diluted with EtOAc (40 mL), washed with H₂O (2×10 mL), dried over Na₂SO₄, concentrated under reduced pressure and purified by RP-prep-HPLC to afford 1-(4-(difluoromethoxy)phenyl)-7-ethoxy-3-(4-methoxyphenyl)-3,4-dihydropyrido[3,2-d]pyrimidin-2(1H)-one (Example 289). LC-MS (ESI): m/z 442 [M+H]⁺.

¹H NMR (400 MHz, DMSO-d₆) δ (ppm): 7.91 (d, J=2.4 Hz, 1H), 7.46 (d, J=8.8 Hz, 2H), 7.38-7.30 (m, 4H), 7.34 (t, J_{HF}=73.8 Hz, 1H), 6.96 (d, J=8.8 Hz, 2H), 5.95 (d, J=2.4 Hz, 1H), 4.91 (s, 2H), 3.95 (q, J=6.8 Hz, 2H), 3.77 (s, 3H), 1.24 (t, J=6.8 Hz, 3H).

The procedure set forth above for General Procedure XI was used to synthesize the following compounds by using appropriate starting materials:

| Cpd No. | Structure | Characterization |
|---|---|---|
| Example 290 | 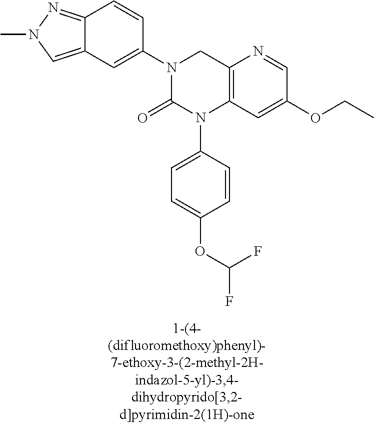 [SMILES: CCOc1cnc2c(c1)N(c1ccc(OC(F)F)cc1)C(=O)N(c1ccc3nn(C)cc3c1)C2] | LC-MS: m/z 466.2 (M + H)+. 1H NMR (400 MHz, DMSO-d₆) δ: 8.34 (s, 1H), 7.92 (d, J = 2.4 Hz, 1H), 7.72 (d, J = 1.2 Hz, 1H), 7.57 (d, J = 8.8 Hz, 1H), 7.48 (d, J = 8.8 Hz, 2H), 7.35 (t, J_{HF} = 74.0 Hz, 1H), 7.33 (d, J = 9.2 Hz, 2H), 7.29 (dd, J = 9.2 Hz, 2.0 Hz, 1H), 5.97 (d, J = 2.4 Hz, 1H), 4.99 (s, 2H), 4.17 (s, 3H), 3.96 (q, J = 6.8 Hz, 2H), 1.25 (t, J = 6.8 Hz, 3H). |
| Example 291 | 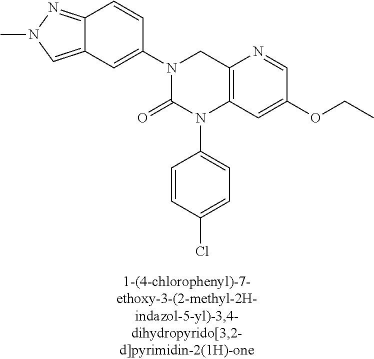 [SMILES: CCOc1cnc2c(c1)N(c1ccc(Cl)cc1)C(=O)N(c1ccc3nn(C)cc3c1)C2] | LC-MS: m/z 434.1 (M + H)+. 1H NMR (400 MHz, DMSO-d₆) δ: 8.34 (s, 1H), 7.92 (d, J = 2.0 Hz, 1H), 7.72 (s, 1H), 7.61 (d, J = 8.8 Hz, 2H), 7.57 (d, J = 9.2 Hz, 1H), 7.46 (d, J = 8.8 Hz, 2H), 7.29 (dd, J = 9.2 Hz, 1.6 Hz, 1H), 6.00 (d, J = 2.0 Hz, 1H), 4.98 (s, 2H), 4.17 (s, 3H), 3.95 (q, J = 6.8 Hz, 2H), 1.25 (t, J = 6.8 Hz, 3H). |

The table below provides a list of prophetic compounds of Formula II that may be synthesized using the General Procedure XI shown above, or a combination of other procedures described herein using ordinary skill.

1-(4-(1H-1,2,4-triazol-3-yl)phenyl)-3-(2-methylbenzo[d]thiazol-6-yl)-7-
((2,2,2-trifluoroethyl)amino)-3,4-dihydropyrido[3,2-d]pyrimidin-2(1H)-one
1-(4-(difluoromethoxy)phenyl)-3-(2-methyl-2H-indazol-5-yl)-7-
((2,2,2-trifluoroethyl)amino)-3,4-dihydropyrido[3,2-d]pyrimidin-2(1H)-one
1-(4-(difluoromethoxy)phenyl)-3-(1-methyl-1H-benzo[d]imidazol-6-yl)-7-
((2,2,2-trifluoroethyl)amino)-3,4-dihydropyrido[3,2-d]pyrimidin-2(1H)-one
1-(4-(difluoromethoxy)phenyl)-7-(ethylamino)-3-(1-methyl-1H-
benzo[d]imidazol-6-yl)-3,4-dihydropyrido[3,2-d]pyrimidin-2(1H)-one
1-(4-(1H-1,2,4-triazol-3-yl)phenyl)-3-(2-methyl-2H-indazol-5-yl)-7-
((2,2,2-trifluoroethyl)amino)-3,4-dihydropyrido[3,2-d]pyrimidin-2(1H)-one
1-(4-(1H-1,2,4-triazol-3-yl)phenyl)-3-(4-methoxyphenyl)-7-
((2,2,2-trifluoroethyl)amino)-3,4-dihydropyrido[3,2-d]pyrimidin-2(1H)-one
1-(4-(4H-1,2,4-triazol-3-yl)phenyl)-7-ethoxy-3-(2-methyl-2H-
indazol-5-yl)-3,4-dihydropyrido[3,2-d]pyrimidin-2(1H)-one
7-ethoxy-1-(6-(fluoromethyl)pyridin-3-yl)-3-(2-methyl-2H-
indazol-5-yl)-3,4-dihydropyrido[3,2-d]pyrimidin-2(1H)-one
7-(2,2-difluoroethoxy)-1-(4-(difluoromethoxy)phenyl)-3-(2-methyl-2H-
indazol-5-yl)-3,4-dihydropyrido[3,2-d]pyrimidin-2(1H)-one
7-(2,2-difluoroethoxy)-1-(4-methoxyphenyl)-3-(2-methyl-2H-
indazol-5-yl)-3,4-dihydropyrido[3,2-d]pyrimidin-2(1H)-one
7-(2,2-difluoroethoxy)-1-(4-(difluoromethoxy)phenyl)-3-(1-methyl-1H-
benzo[d]imidazol-6-yl)-3,4-dihydropyrido[3,2-d]pyrimidin-2(1H)-one
7-(2,2-difluoroethoxy)-1-(4-methoxyphenyl)-3-(1-methyl-1H-
benzo[d]imidazol-6-yl)-3,4-dihydropyrido[3,2-d]pyrimidin-2(1H)-one
7-ethoxy-1-(4-(methoxy-d3)phenyl)-3-(2-methyl-2H-indazol-5-yl)-3,4-
dihydropyrido[3,2-d]pyrimidin-2(1H)-one
7-(2,2-difluoroethoxy)-1-(4-(methoxy-d3)phenyl)-3-(2-methyl-2H-
indazol-5-yl)-3,4-dihydropyrido[3,2-d]pyrimidin-2(1H)-one
7-(2,2-difluoroethoxy)-1-(4-(methoxy-d3)phenyl)-3-(1-methyl-1H-
benzo[d]imidazol-6-yl)-3,4-dihydropyrido[3,2-d]pyrimidin-2(1H)-one
7-(2,2-difluoroethoxy)-3-(2,3-dimethyl-2H-indazol-5-yl)-1-(4-
(methoxy-d3)phenyl)-3,4-dihydropyrido[3,2-d]pyrimidin-2(1H)-one
7-(2,2-difluoroethoxy)-3-(3-methoxy-2-methyl-2H-indazol-5-yl)-1-(4-
(methoxy-d3)phenyl)-3,4-dihydropyrido[3,2-d]pyrimidin-2(1H)-one
7-(2,2-difluoroethoxy)-3-(3-(methoxy-d3)-2-methyl-2H-indazol-5-yl)-1-(4-
(methoxy-d3)phenyl)-3,4-dihydropyrido[3,2-d]pyrimidin-2(1H)-one
1-(4-bromophenyl)-7-(2,2-difluoroethoxy)-3-(2-methyl-2H-
indazol-5-yl)-3,4-dihydropyrido[3,2-d]pyrimidin-2(1H)-one
1-(4-methoxyphenyl)-3-(2-methyl-2H-indazol-5-yl)-7-
propyl-3,4-dihydropyrido[3,2-d]primidin-2(1H)-one
7-isopropoxy-1-(4-(methoxy-d3)phenyl)-3-(2-methyl-2H-
indazol-5-yl)-3,4-dihydropyrido[3,2-d]pyrimidin-2(1H)-one
7-(2,2-difluoroethoxy)-3-(2-ethyl-2H-indazol-5-yl)-1-(4-
(methoxy-d3)phenyl)-3,4-dihydropyrido[3,2-d]pyrimidin-2(1H)-one
7-(2,2-difluoroethoxy)-1-(4-(methoxy-d3)phenyl)-3-(2-(2-methoxyethyl)-
2H-indazol-5-yl)-3,4-dihydropyrido[3,2-d]pyrimidin-2(1H)-one
7-(2,2-difluoroethoxy)-1-(1H-indol-5-yl)-3-(2-methyl-2H-
indazol-5-yl)-3,4-dihydropyrido[3,2-d]pyrimidin-2(1H)-one
1-(benzo[d]thiazol-5-yl)-7-(2,2-difluoroethoxy)-3-(2-methyl-2H-
indazol-5-yl)-3,4-dihydropyrido[3,2-d]pyrimidin-2(1H)-one
7-(2,2-difluoroethoxy)-1-(1-methyl-1H-indol-5-yl)-3-(2-methyl-2H-
indazol-5-yl)-3,4-dihydropyrido[3,2-d]pyrimidin-2(1H)-one
7-((1,1-difluoropropan-2-yl)oxy)-1-(4-(methoxy-d3)phenyl)-3-(2-methyl-
2H-indazol-5-l)-3,4-dihydropyrido[3,2-d]pyrimidin-2(1H)-one
1-(4-(difluoromethoxy)phenyl)-7-ethoxy-3-(2-methyl-2H-
indazol-5-yl)-3,4-dihydropyrido[3,2-d]pyrimidin-2(1H)-one

### General Procedure XIH

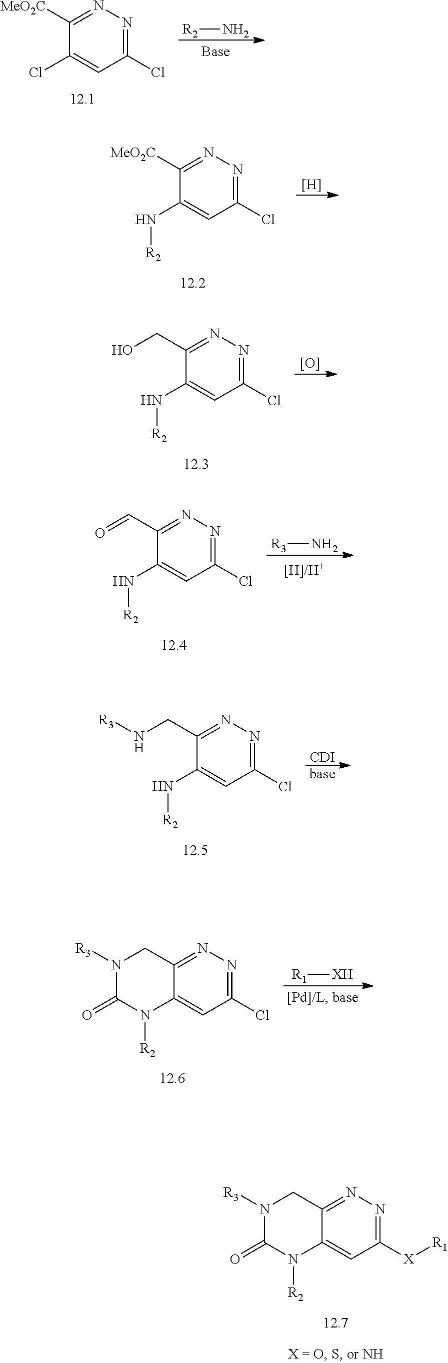

Compounds of structure 12.7 may be obtained through the scheme depicted as General Procedure XII. Beginning with aryl chloride 12.1, the desired R₂ group may be introduced through a base mediated aromatic substitution to generate compound 12.2. Aryl ester 12.2 may then be reduced with a hydride source to generate alcohol 12.3. Alcohol 12.3 may then be oxidized up to aldehyde 12.4. The desired R₃ group may be introduced through a reductive amination with aldehyde 12.4 to generate diamine 12.5. Diamine 12.5 may then be cyclized using CDI to generate cyclic urea 12.6. Lastly, the desired R₁ group may be introduced with a palladium mediated C—X coupling reaction to afford compounds of structure 12.7.

### Preparation of Example 292 Via General Procedure XII

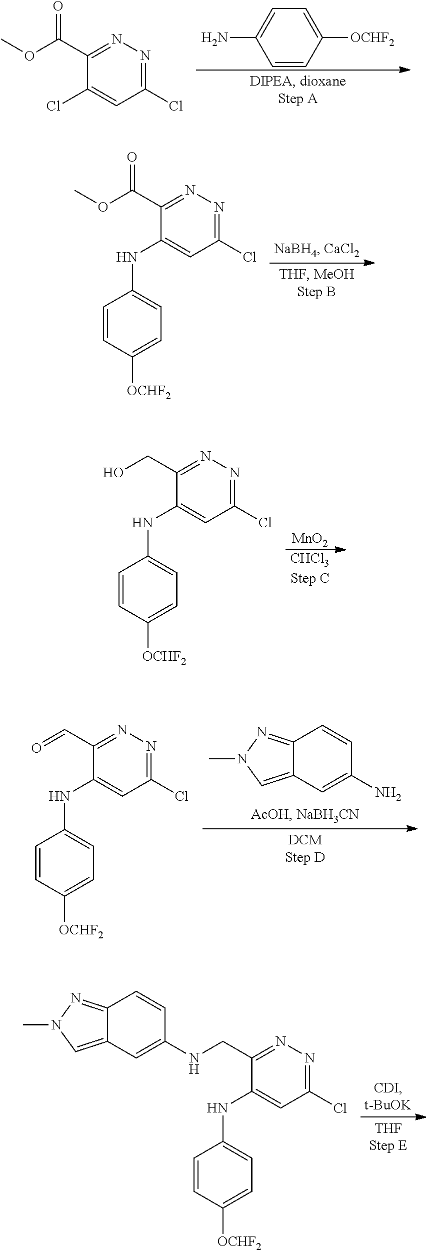

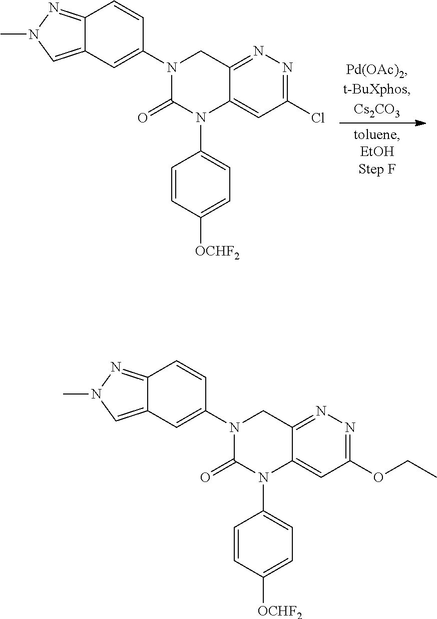 [SMILES: Cn1cc2cc(N3Cc4nnc(Cl)cc4N(c4ccc(OC(F)F)cc4)C3=O)ccc2n1 ; CCOc1cc2c(nn1)CN(c1ccc3nn(C)cc3c1)C(=O)N2c1ccc(OC(F)F)cc1]

### Step A: methyl 6-chloro-4-((4-(difluoromethoxy)phenyl)amino)pyridazine-3-carboxylate

To a solution of methyl 4,6-dichloropyridazine-3-carboxylate (1.0 g, 4.8 mmol, 1.0 eq.) in dioxane (15 mL) was added 4-(difluoromethoxy)aniline (850 mg, 5.3 mmol, 1.1 eq.) and DIPEA (1.370 g, 10.6 mmol, 2.2 eq.), the reaction mixture was stirred at 100° C. for 20 hrs. Then the reaction mixture was quenched with ice water (40 mL) and extracted with EtOAc (100 mL×3). The combined organic layers were washed with brine (50 mL), dried over Na₂SO₄ and concentrated under reduced pressure, the residue was purified by column chromatography to afford methyl 6-chloro-4-((4-(difluoromethoxy)phenyl)amino)pyridazine-3-carboxylate (1100 mg, 69% yield) as a white solid. LC-MS (ESI): m/z 330 [M+H]⁺.

### Step B: (6-chloro-4-((4-(difluoromethoxy)phenyl)amino)pyridazin-3-yl)methanol

To a solution of methyl 6-chloro-4-((4-(difluoromethoxy)phenyl)amino)pyridazine-3-carboxylate (500 mg, 1.5 mmol, 1.0 eq.) in THF (6 mL) and MeOH (4 mL) was added NaBH₄ (288 mg, 7.5 mmol, 5.0 eq.) and CaCl₂) (370 mg, 3.0 mmol, 2.0 eq.) at 0° C. The reaction mixture was allowed to warm to room temperature and stirred for 2 hrs. The progress of the reaction was monitored by LC-MS (ESI), after completion, the reaction mixture was quenched with ice water (20 mL) and extracted with EtOAc (50 mL×3). The combined organic layers were washed with brine (50 mL), dried over Na₂SO₄ and concentrated under reduced pressure, the residue was purified by column chromatography to afford (6-chloro-4-((4-(difluoromethoxy)phenyl)amino)pyridazin-3-yl)methanol (420 mg, 92% yield) as a white solid. LC-MS (ESI): m/z 302 [M+H]⁺.

### Step C: 6-chloro-4-((4-(difluoromethoxy)phenyl)amino)pyridazine-3-carbaldehyde

To a solution of (6-chloro-4-((4-(difluoromethoxy)phenyl)amino)pyridazin-3-yl)methanol (420 mg, 1.4 mmol, 1.0 eq.) in CHCl₃ (10 mL) was added MnO₂ (1.21 g, 14.0 mmol, 10.0 eq.), the reaction mixture was stirred at room temperature for 16 hrs. The progress of the reaction was monitored by LC-MS, after completion, MnO₂ was removed by filtering through a short pad of Celite®, the filtrate was concentrated under reduced pressure to afford crude 6-chloro-4-((4-(difluoromethoxy)phenyl)amino)pyridazine-3-carbaldehyde (380 mg) as a white solid, which was used in next step without further purification. LC-MS (ESI): m/z 300 [M+H]⁺.

### Step D: N-((6-chloro-4-((4-(difluoromethoxy)phenyl)amino)pyridazin-3-yl)methyl)-2-methyl-2H-indazol-5-amine

To a solution of 6-chloro-4-((4-(difluoromethoxy)phenyl)amino)pyridazine-3-carbaldehyde (380 mg, 1.3 mmol, 1.0 eq.) in DCM (10 mL) was added 2-methyl-2H-indazol-5-amine (190 mg, 1.3 mmol, 1.0 eq.) and AcOH (80 mg, 1.3 mmol, 1.0 eq.), the reaction mixture was stirred at room temperature for 1 h. Then the reaction mixture was cooled to 0° C., NaBH₃CN (82 mg, 1.3 mmol, 1.0 eq.) was added, the reaction mixture was allowed to warm to room temperature and stirred for 1 h. The progress of the reaction was monitored by LC-MS, after completion, the reaction was quenched with ice water (15 mL) and extracted with DCM (50 mL×2). The combined organic layers were washed with brine (20 mL), dried over Na₂SO₄ and concentrated under reduced pressure, the residue was purified by column chromatography to afford N-((6-chloro-4-((4-(difluoromethoxy)phenyl)amino)pyridazin-3-yl)methyl)-2-methyl-2H-indazol-5-amine (480 mg, 88% yield) as a white solid. LC-MS(ESI): m/z 431 [M+H]⁺.

### Step E: 3-chloro-5-(4-(difluoromethoxy)phenyl)-7-(2-methyl-2H-indazol-5-yl)-7,8-dihydropyrimido[5,4-c]pyridazin-6(5H)-one

To a solution of N-((6-chloro-4-((4-(difluoromethoxy)phenyl)amino)pyridazin-3-yl)methyl)-2-methyl-2H-indazol-5-amine (480 mg, 1.1 mmol, 1.0 eq.) in THF (10 mL) was added CDI (360 mg, 2.1 mmol, 2.0 eq.) and t-BuOK (250 mg, 2.2 mmol, 2.0 eq.), the reaction mixture was stirred at 60° C. for 2 hrs. The progress of the reaction was monitored by LC-MS, after completion, the reaction mixture was quenched with ice water (15 mL) and extracted with EtOAc (50 mL×3). The combined organic layers were washed with brine (50 mL), dried over Na₂SO₄ and concentrated under reduced pressure, the residue was purified by column chromatography to afford 3-chloro-5-(4-(difluoromethoxy)phenyl)-7-(2-methyl-2H-indazol-5-yl)-7,8-dihydropyrimido[5,4-c]pyridazin-6(5H)-one (450 mg, 88% yield) as a white solid. LC-MS (ESI): m/z 457 [M+H]⁺.

### Step F: 5-(4-(difluoromethoxy)phenyl)-3-ethoxy-7-(2-methyl-2H-indazol-5-yl)-7,8-dihydropyrimido[5,4-c]pyridazin-6(5H)-one

A mixture of 3-chloro-5-(4-(difluoromethoxy)phenyl)-7-(2-methyl-2H-indazol-5-yl)-7,8-dihydropyrimido[5,4-c]pyridazin-6(5H)-one (50 mg, 0.11 mmol, 1.0 eq.), Pd(OAc)₂ (2.44 mg, 0.011 mmol, 0.1 eq.), t-BuXPhos (10 mg, 0.022 mmol 0.2 eq.) and Cs₂CO₃ (107 mg, 0.33 mmol, 3.0 eq.) in EtOH (2 mL) and toluene (2 mL) was stirred under N₂ atmosphere at 100° C. for 15 hrs. The progress of the reaction was monitored by LC-MS, after completion, the reaction mixture was diluted with water (10 mL) and extracted with EtOAc (20 mL×3). The combined organic layers were dried over Na₂SO₄ and concentrated under reduced pressure, the residue was purified by column chromatography and Prep-HPLC to afford 5-(4-(difluoromethoxy)phenyl)-3-ethoxy-7-(2-methyl-2H-indazol-5-yl)-7,8-dihydropyrimido[5,4-c]pyridazin-6(5H)-one (Example 292). LC-MS (ESI): m/z=467 [M+H]⁺.

¹H NMR (400 MHz, DMSO-d₆) δ: 8.37 (s, 1H), 7.78 (d, J=1.6 Hz, 1H), 7.60 (d, J=9.6 Hz, 1H), 7.50 (d, J=8.4 Hz, 2H), 7.36 (d, J=8.8 Hz, 2H), 7.34 (t, J_{HF}=74.0 Hz, 1H), 7.31 (dd, J=9.6 Hz, 2.0 Hz, 1H), 5.54 (s, 1H), 5.17 (s, 2H), 4.40 (q, J=6.8 Hz, 2H), 4.18 (s, 3H), 1.29 (t, J=6.8 Hz, 3H).

The procedure set forth above for General Procedure XII was used to synthesize the following compounds by using appropriate starting materials:

| Cpd No. | Structure | Characterization |
|---|---|---|
| Example 293 | 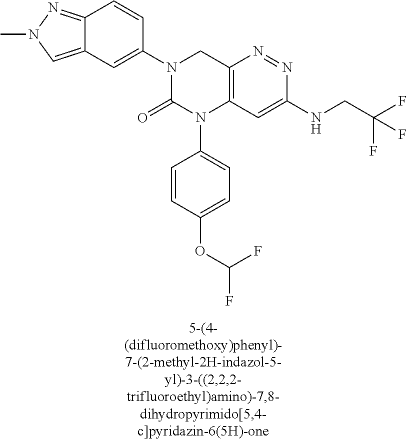 [SMILES: Cn1cc2cc(N3Cc4nnc(NCC(F)(F)F)cc4N(c4ccc(OC(F)F)cc4)C3=O)ccc2n1] | LC-MS: m/z 520.1 (M + H)+. 1H NMR (400 MHz, DMSO-d₆) δ: 8.36 (s, 1H), 7.75 (d, J = 1.6 Hz, 1H), 7.59 (d, J = 9.2 Hz, 1H), 7.50 (d, J = 8.8 Hz, 2H), 7.37 (d, J = 8.8 Hz, 2H), 7.36 (t, J_{HF} = 74.0 Hz, 1H), 7.30 (dd, J = 9.2 Hz, 2.0 Hz, 1H), 7.23 (t, J = 6.4 Hz, 1H), 5.65 (s, 1H), 5.07 (s, 2H), 4.30-4.18 (m, 2H), 4.17 (s, 3H). |
| Example 438 | 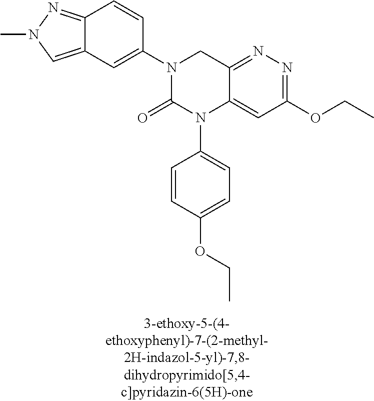 [SMILES: CCOc1ccc(N2C(=O)N(c3ccc4nn(C)cc4c3)Cc3nnc(OCC)cc32)cc1] | LC-MS: m/z 445 (M + H)+. 1H NMR (400 MHz, DMSO-d₆) δ: 8.37 (s, 1H), 7.77 (s, 1H), 7.59 (d, J = 9.2 Hz, 1H), 7.31 (d, J = 8.4 Hz, 3H), 7.08 (d, J = 8.0 Hz, 2H), 5.51 (s, 1H), 5.16 (s, 2H), 4.39 (q, J = 6.8 Hz, 2H), 4.18 (s, 3H), 4.09 (q, J = 6.8 Hz, 2H), 1.36 (t, J = 6.8 Hz, 3H), 1.29 (t, J = 6.8 Hz, 3H). |

The table below provides a list of prophetic compounds of Formula II that may be synthesized using the General Procedure XII shown above, or a combination of other procedures described herein using ordinary skill.

5-(4-(1H-1,2,4-triazol-3-yl)phenyl)-7-(2-methylbenzo[d]thiazol-6-yl)-3-
((2,2,2-trifluoroethyl)amino)-7,8-dihydropyrimido[5,4-c]pyridazin-6(5H)-one
5-(4-(difluoromethoxy)phenyl)-7-(1-methyl-1H-benzo[d]imidazol-6-yl)-3-
((2,2,2-trifluoroethyl)amino)-7,8-dihydropyrimido[5,4-c]pyridazin-6(5H)-one
5-(4-(difluoromethoxy)phenyl)-3-(ethylamino)-7-(1-methyl-1H-benzo[d]imidazol-6-yl)-
7,8-dihydropyrimido[5,4-c]pyridazin-6(5H)-one
5-(4-(1H-1,2,4-triazol-3-yl)phenyl)-7-(2-methyl-2H-indazol-5-yl)-3-
((2,2,2-trifluoroethyl)amino)-7,8-dihydropyrimido[5,4-c]pyridazin-6(5H)-one
5-(4-(1H-1,2,4-triazol-3-yl)phenyl)-7-(4-methoxyphenyl)-3-
((2,2,2-trifluoroethyl)amino)-7,8-dihydropyrimido[5,4-c]pyridazin-6(5H)-one
5-(4-(4H-1,2,4-triazol-3-yl)phenyl)-3-ethoxy-7-(2-methyl-2H-
indazol-5-yl)-7,8-dihydropyrimido[5,4-c]pyridazin-6(5H)-one
3-ethoxy-5-(6-(fluoromethyl)pyridin-3-yl)-7-(2-methyl-2H-
indazol-5-yl)-7,8-dihydropyrimido[5,4-c]pyridazin-6(5H)-one
3-(2,2-difluoroethoxy)-5-(4-(difluoromethoxy)phenyl)-7-(2-methyl-2H-
indazol-5-yl)-7,8-dihydropyrimido[5,4-c]pyridazin-6(5H)-one
3-(2,2-difluoroethoxy)-5-(4-methoxyphenyl)-7-(2-methyl-2H-
indazol-5-yl)-7,8-dihydropyrimido[5,4-c]pyridazin-6(5H)-one
3-(2,2-difluoroethoxy)-5-(4-(difluoromethoxy)phenyl)-7-(1-methyl-1H-
benzo[d]imidazol-6-yl)-7,8-dihydropyrimido[5,4-c]pyridazin-6(5H)-one
3-(2,2-difluoroethoxy)-5-(4-methoxyphenyl)-7-(1-methyl-1H-
benzo[d]imidazol-6-yl)-7,8-dihydropyrimido[5,4-c]pyridazin-6(5H)-one
3-ethoxy-5-(4-(methoxy-d3)phenyl)-7-(2-methyl-2H-indazol-5-yl)-7,8-
dihydropyrimido[5,4-c]pyridazin-6(5H)-one
3-(2,2-difluoroethoxy)-5-(4-(methoxy-d3)phenyl)-7-(2-methyl-2H-
indazol-5-yl)-7,8-dihydropyrimido[5,4-c]pyridazin-6(5H)-one
3-(2,2-difluoroethoxy)-5-(4-(methoxy-d3)phenyl)-7-(1-methyl-1H-
benzo[d]imidazol-6-yl)-7,8-dihydropyrimido[5,4-c]pyridazin-6(5H)-one
3-(2,2-difluoroethoxy)-7-(2,3-dimethyl-2H-indazol-5-yl)-5-(4-
(methoxy-d3)phenyl)-7,8-dihydropyrimido[5,4-c]pyridazin-6(5H)-one
3-(2,2-difluoroethoxy)-7-(3-methoxy-2-methyl-2H-indazol-5-yl)-5-(4-
(methoxy-d3)phenyl)-7,8-dihydropyrimido[5,4-c]pyridazin-6(5H)-one
3-(2,2-difluoroethoxy)-7-(3-(methoxy-d3)-2-methyl-2H-indazol-5-yl)-5-(4-
(methoxy-d3)phenyl)-7,8-dihydropyrimido[5,4-c]pyridazin-6(5H)-one
5-(4-bromophenyl)-3-(2,2-difluoroethoxy)-7-(2-methyl-2H-
indazol-5-yl)-7,8-dihydropyrimido[5,4-c]pyridazin-6(5H)-one
5-(4-methoxyphenyl)-7-(2-methyl-2H-indazol-5-yl)-3-
propyl-7,8-dihydropyrimido[5,4-c]pyridazin-6(5H)-one
3-isopropoxy-5-(4-(methoxy-d3)phenyl)-7-(2-methyl-2H-
indazol-5-yl)-7,8-dihydropyrimido[5,4-c]pyridazin-6(5H)-one
3-(2,2-difluoroethoxy)-7-(2-ethyl-2H-indazol-5-yl)-5-(4-
(methoxy-d3)phenyl)-7,8-dihydropyrimido[5,4-c]pyridazin-6(5H)-one
3-(2,2-difluoroethoxy)-5-(4-(methoxy-d3)phenyl)-7-(2-
(2-methoxyethyl)-2H-indazol-5-yl)-7,8-dihydropyrimido[5,4-c]pyridazin-6(5H)-one
3-(2,2-difluoroethoxy)-5-(1H-indol-5-yl)-7-(2-methyl-2H-
indazol-5-yl)-7,8-dihydropyrimido[5,4-c]pyridazin-6(5H)-one
5-(benzo[d]thiazol-5-yl)-3-(2,2-difluoroethoxy)-7-(2-methyl-2H-
indazol-5-yl)-7,8-dihydropyrimido[5,4-c]pyridazin-6(5H)-one
3-(2,2-difluoroethoxy)-5-(1-methyl-1H-indol-5-yl)-7-(2-methyl-2H-
indazol-5-yl)-7,8-dihydropyrimido[5,4-c]pyridazin-6(5H)-one
3-((1,1-difluoropropan-2-yl)oxy)-5-(4-(methoxy-d3)phenyl)-7-
(2-methyl-2H-indazol-5-yl)-7,8-dihydropyrimido[5,4-c]pyridazin-6(5H)-one

The table below provides a list of prophetic compounds of Formula II that may be synthesized using the General Procedure III (Case IV), General Procedure IX (Case II), or a combination of other procedures described herein using ordinary skill.

1-(4-(1H-1,2,4-triazol-3-yl)phenyl)-3-(2-methylbenzo[d]thiazol-6-yl)-7-
((2,2,2-trifluoroethyl)amino)-3,4-dihydropyrido[4,3-d]pyrimidin-2(1H)-one
1-(4-(difluoromethoxy)phenyl)-3-(1-methyl-1H-benzo[d]imidazol-6-yl)-7-
((2,2,2-trifluoroethyl)amino)-3,4-dihydropyrido[4,3-d]pyrimidin-2(1H)-one
1-(4-(difluoromethoxy)phenyl)-7-(ethylamino)-3-(1-methyl-1H-benzo[d]
imidazol-6-yl)-3,4-dihydropyrido[4,3-d]pyrimidin-2(1H)-one
1-(4-(1H-1,2,4-triazol-3-yl)phenyl)-3-(2-methyl-2H-indazol-5-yl)-7-
((2,2,2-trifluoroethyl)amino)-3,4-dihydropyrido[4,3-d]pyrimidin-2(1H)-one
1-(4-(1H-1,2,4-triazol-3-yl)phenyl)-3-(4-methoxyphenyl)-7-
((2,2,2-trifluoroethyl)amino)-3,4-dihydropyrido[4,3-d]pyrimidin-2(1H)-one
1-(4-(4H-1,2,4-triazol-3-yl)phenyl)-7-ethoxy-3-(2-methyl-2H-
indazol-5-yl)-3,4-dihydropyrido[4,3-d]pyrimidin-2(1H)-one
7-ethoxy-1-(6-(fluoromethyl)pyridin-3-yl)-3-(2-methyl-2H-
indazol-5-yl)-3,4-dihydropyrido[4,3-d]pyrimidin-2(1H)-one
7-(2,2-difluoroethoxy)-1-(4-(difluoromethoxy)phenyl)-3-
(2-methyl-2H-indazol-5-yl)-3,4-dihydropyrido[4,3-d]pyrimidin-2(1H)-one
7-(2,2-difluoroethoxy)-1-(4-methoxyphenyl)-3-(2-methyl-2H-
indazol-5-yl)-3,4-dihydropyrido[4,3-d]pyrimidin-2(1H)-one
7-(2,2-difluoroethoxy)-1-(4-(difluoromethoxy)phenyl)-3-
(1-methyl-1H-benzo[d]imidazol-6-yl)-3,4-dihydropyrido[4,3-d]pyrimidin-2(1H)-one
7-(2,2-difluoroethoxy)-1-(4-methoxyphenyl)-3-(1-methyl-1H-
benzo[d]imidazol-6-yl)-3,4-dihydropyrido[4,3-d]pyrimidin-2(1H)-one
7-ethoxy-1-(4-(methoxy-d3)phenyl)-3-(2-methyl-2H-indazol-5-yl)-3,4-
dihydropyrido[4,3-d]pyrimidin-2(1H)-one
7-(2,2-difluoroethoxy)-1-(4-(methoxy-d3)phenyl)-3-(2-methyl-2H-
indazol-5-yl)-3,4-dihydropyrido[4,3-d]pyrimidin-2(1H)-one
7-(2,2-difluoroethoxy)-1-(4-(methoxy-d3)phenyl)-3-(1-methyl-1H-
benzo[d]imidazol-6-yl)-3,4-dihydropyrido[4,3-d]pyrimidin-2(1H)-one
7-(2,2-difluoroethoxy)-3-(2,3-dimethyl-2H-indazol-5-yl)-1-(4-
(methoxy-d3)phenyl)-3,4-dihydropyrido[4,3-d]pyrimidin-2(1H)-one
7-(2,2-difluoroethoxy)-3-(3-methoxy-2-methyl-2H-indazol-5-yl)-1-(4-
(methoxy-d3)phenyl)-3,4-dihydropyrido[4,3-d]pyrimidin-2(1H)-one
7-(2,2-difluoroethoxy)-3-(3-(methoxy-d3)-2-methyl-2H-indazol-5-yl)-1-(4-
(methoxy-d3)phenyl)-3,4-dihydropyrido[4,3-d]pyrimidin-2(1H)-one
1-(4-bromophenyl)-7-(2,2-difluoroethoxy)-3-(2-methyl-2H-
indazol-5-yl)-3,4-dihydropyrido[4,3-d]pyrimidin-2(1H)-one
1-(4-methoxyphenyl)-3-(2-methyl-2H-indazol-5-yl)-7-propyl-3,4-
dihydropyrido[4,3-d]pyrimidin-2(1H)-one
7-isopropoxy-1-(4-(methoxy-d3)phenyl)-3-(2-methyl-2H-
indazol-5-yl)-3,4-dihydropyrido[4,3-d]pyrimidin-2(1H)-one
7-(2,2-difluoroethoxy)-3-(2-ethyl-2H-indazol-5-yl)-1-(4-
(methoxy-d3)phenyl)-3,4-dihydropyrido[4,3-d]pyrimidin-2(1H)-one
7-(2,2-difluoroethoxy)-1-(4-(methoxy-d3)phenyl)-3-(2-(2-methoxyethyl)-
2H-indazol-5-yl)-3,4-dihydropyrido[4,3-d]pyrimidin-2(1H)-one
7-(2,2-difluoroethoxy)-1-(1H-indol-5-yl)-3-(2-methyl-2H-
indazol-5-yl)-3,4-dihydropyrido[4,3-d]pyrimidin-2(1H)-one
7-(2,2-difluoroethoxy)-1-(4-(methoxy-d3)phenyl)-5-methyl-3-(2-methyl-
2H-indazol-5-yl)-3,4-dihydropyrido[4,3-d]pyrimidin-2(1H)-one
1-(4-methoxyphenyl)-5-methyl-3-(2-methyl-2H-indazol-5-yl)-7-
((2,2,2-trifluoroethyl)amino)-3,4-dihydropyrido[4,3-d]pyrimidin-2(1H)-one
1-(benzo[d]thiazol-5-yl)-7-(2,2-difluoroethoxy)-3-(2-methyl-2H-
indazol-5-yl)-3,4-dihydropyrido[4,3-d]pyrimidin-2(1H)-one
7-(2,2-difluoroethoxy)-1-(1-methyl-1H-indol-5-yl)-3-(2-methyl-2H-
indazol-5-yl)-3,4-dihydropyrido[4,3-d]pyrimidin-2(1H)-one
7-((1,1-difluoropropan-2-yl)oxy)-1-(4-(methoxy-d3)phenyl)-3-(2-methyl-
2H-indazol-5-yl)-3,4-dihydropyrido[4,3-d]pyrimidin-2(1H)-one

Biochemical Assay

Mat2A protein was expressed by recombinant baculovirus in SF9 infected cells using the Bac to Bac system cloned into the pFASTBAC1 vector (Invitrogen, Carlsbad, Calif.). Recombinant MAT2A was isolated from the cell lysate of 150 g of infected cells using HP Ni sepharose column chromatography. Recombinant MAT2A homodimer was eluted with 250 and 500 mM imidazole, and fractions containing MAT2A were identified by sodium dodecyl sulfate polyacrylamide gel electrophoresis and pooled.

For determination of the inhibitory potency of compounds against the MAT2A homodimer, protein was diluted to 4 μg/mL in assay buffer (50 mM Tris, pH 8.0, 50 mM KCl, 15 mM MgCl2, 0.3 mM EDTA, 0.005% [w/v] bovine serum albumin [BSA]). Test compound was prepared in 100% dimethyl sulfoxide (DMSO) at 50× the desired final concentration. A 1 μL volume of compound dilution was added to 40 μL of enzyme dilution and the mixture was allowed to equilibrate for 60 minutes at 25° C. The enzymatic assay was initiated by the addition of 10 μL of substrate mix (500 μM ATP, pH 7.0, 400 μM L-methionine in 1× assay buffer), and the mixture was incubated for a further 60 minutes at 25° C. The reaction was halted and the liberated phosphate released by the enzyme in stoichiometric amounts by the production of S-adenosyl methionine (SAM) was measured using the PiColorLock Gold kit (Innova Biosciences, UK). Absolute product amounts were determined by comparison to a standard curve of potassium phosphate buffer, pH 8.0.

Specific compounds disclosed herein were tested in the foregoing assay and they were determined to inhibit MAT2A with an IC₅₀ according to the following scores: (A) less than 100 nM (>40% maximum inhibition), (B) between 100 nM and 1 μM (>38% maximum inhibition), (C) between 1 μM and 10 μM (>40% maximum inhibition), and (D) greater than 10 μM as shown in Table 5 below.

Cellular Assay of Target Engagement (SAM)

Measurement of MAT2A activity in cells was made by direct quantitation of the abundance of the product of its enzymatic activity, SAM. Cancer cells were treated with candidate MAT2A inhibitors for a suitable incubation period, and the cells were then lysed using a reagent which quenched any further enzyme activity. Soluble metabolites including SAM were collected and SAM itself was directly measured from the lysate using quantitative LC-MS/MS.

A typical assay was performed using an HCT116 human colon carcinoma cell line which was genetically engineered to delete the MTAP gene (commercially available from Horizon Discovery). This cell line was utilized because it was determined that loss of the MTAP gene predicts sensitivity to MAT2A inhibitors. Cells were plated in 96-well dishes at appropriate cell density. Following 24 hours, cells were then treated with the candidate MAT2A inhibitor. Prior to addition to cells, the compound was first serially diluted in 100% DMSO, typically as a 3-fold serial dilution starting at 500× top dose with 10 dose points including DMSO only control. Compound was then transferred to a working stock plate in cell culture media by adding 5 μL of compound in DMSO to 495 μL of cell culture media. This working stock was then added to cells via a further 5-fold dilution, by adding 25 μL of working stock to 100 μL of cells in culture media. Following compound addition, cells were incubated at 37° C./5% CO₂ for 72 hrs.

To quantitate SAM levels following compound treatment, cells were gently washed once in ammonium carbonate buffer (75 mM at pH 7.4), placed on dry ice, and lysed with metabolite extraction buffer (80% cold methanol and 20% water (v/v) with acetic acid at 1M final concentration with 200 ng/mL deuterated d3-SAM as internal control). Following centrifugation at 4° C. at 3,200 rpm for 30 minutes, the supernatant was collected and stored at −80° C. until analysis by Liquid Chromatography with tandem Mass Spectrometry (LC-MS/MS). LC-MS/MS analysis was performed using an API6500 Mass Spectrometer (Sciex, Framingham, Mass., USA) operating in positive ion spray mode and equipped with a Waters UPLC Acquity (Waters, Milford, Mass., USA) BEH Amide column. Multiple Reaction Monitoring data was acquired for SAM and the d3-SAM standard, using a mass transition pair at m/z 399.2→250.1 and 402.2→250.1, respectively. In a typical LC-MS/MS analysis, the initial flow rate was 0.5 ml/min of 25% mobile phase A (acetonitrile and water at 5:95 (v/v) with 1% formic acid and 10 mM ammonium acetate) and 75% mobile phase B (acetonitrile and water at 95:5 (v/v) with 1% formic acid and 10 mM ammonium acetate), 0.2-0.5 minutes with 75%-35% mobile phase B, 25%-65% mobile phase A, at 0.5 min 65% mobile phase A and 35% mobile phase B, 1.0-1.1 minutes with 35%-75% mobile phase B, 65%-25% mobile phase A, at 1.1 min 25% mobile phase A and 75% mobile phase B with a total run time of 1.5 minutes.

Specific compounds disclosed herein were tested in the foregoing assay and they were determined to inhibit SAM with an IC₅₀ according to the following scores: (A) less than 100 nM (>60% maximum inhibition), (B) between 100 nM and 1 μM (>60% maximum inhibition), (C) greater than or equal to 1 μM (>60% maximum inhibition), and (NT) not tested, as shown in Table 5 below.

Assay for Inhibition of Cellular Proliferation

Test compound impact on cancer cell growth was assessed by treating cancer cells with compound for 4 days and then measuring proliferation using an ATP-based cell proliferation readout (Cell Titer Glo, Promega Corporation).

In a typical assay an isogenic pair of HCT116 human colon carcinoma cell lines which vary only in MTAP deletion status (HCT116 MTAP+/+ and HCT116 MTAP−/−) were plated in 96-well dishes at appropriate cell density. Following 24 hours, cells were then treated with the candidate MAT2A inhibitor. Prior to addition to cells, the compound was first serially diluted in 100% DMSO, typically as a 3-fold serial dilution starting at 500× top dose with 10 dose points including DMSO only control. Compound was then transferred to a working stock plate in cell culture media by adding 5 μL of compound in DMSO to 495 μL of cell culture media. This working stock was then added to cells via a further 5-fold dilution, by adding 25 μL of working stock to 100 μL of cells in culture media. Following compound addition, cells were incubated at 37° C./5% CO2 for 4 days.

To measure inhibition of cellular proliferation, cells were allowed to equilibrate to room temperature for 30 minutes, and were then treated with 125 μL of Cell Titer Glo reagent. The plate was then covered with aluminum foil and shaken for 15 minutes to ensure complete mixing and full cell lysis. Luminescent signal was then measured using a plate-based luminometer Veritas version 1.9.2 using ATP standard curve to confirm assay reproducibility from run to run. This luminescence measure was converted to a proliferation index by subtracting from each data point the ATP luminescence signal measured from a bank (no cells) well and dividing by the ATP luminescence signal measured in 0.2% DMSO control well adjusted for signal in blank well. Compound activity was then represented as a percentage change in proliferation relative to a within-plate DMSO control against log 10 of compound concentration in molar (M) units.

Specific compounds disclosed herein were tested in the foregoing assay and they were determined to inhibit cellular proliferation with an IC₅₀ according to the following scores: (A) less than 100 nM (>30% maximum inhibition for MTAP−/−; >10% maximum inhibition for MTAP+/+), (B) between 100 nM and 1 μM (>30% maximum inhibition for MTAP−/−; >10% maximum inhibition for MTAP+/+), (C) greater than or equal to 1 μM, and (NT) not tested, as shown in Table 5a and Table 5b below.

TABLE 5a

| | | Cell | 4 Day | 4 Day |
|---|---|---|---|---|
| | | 72 h | Relative | Relative |
| | | SAM | Growth | Growth |
| | Enzyme | Inhibition | Inhibition | Inhibition |
| Example | Inhibition | (MTAP −/−) | (MTAP −/−) | (MTAP +/+) |
| 101 | A | B | B | C |
| 102 | A | A | B | C |
| 103 | A | A | A | C |
| 104 | A | A | A | B |
| 105 | A | A | A | B |
| 106 | A | A | B | C |
| 107 | A | B | B | C |
| 108 | A | B | B | C |
| 109 | A | A | B | C |
| 110 | A | A | B | C |
| 111 | A | NT | NT | NT |
| 112 | A | B | B | C |
| 113 | A | A | A | C |
| 114 | A | A | A | C |
| 115 | A | B | A | C |
| 116 | C | NT | NT | NT |
| 117 | B | NT | NT | NT |
| 118 | A | B | A | C |
| 119 | A | A | B | C |
| 120 | A | B | B | C |
| 121 | C | NT | NT | NT |
| 122 | A | B | B | C |
| 123 | A | A | B | C |
| 124 | A | A | A | C |
| 125 | A | A | A | C |
| 126 | B | NT | NT | NT |
| 127 | B | NT | NT | NT |
| 128 | A | B | B | C |
| 129 | A | B | B | C |
| 130 | A | B | B | C |
| 131 | A | A | B | B |
| 132 | A | B | B | C |
| 133 | A | A | A | C |
| 135 | A | A | A | C |
| 136 | A | A | A | B |
| 137 | A | A | A | C |
| 138 | A | A | B | C |
| 139 | A | A | A | C |
| 140 | A | A | A | C |
| 141 | A | A | A | B |
| 142 | A | A | B | C |
| 143 | A | A | B | C |
| 144 | A | A | B | C |
| 145 | A | A | A | C |
| 146 | A | A | B | C |
| 147 | A | B | B | C |
| 148 | B | NT | NT | NT |
| 149 | A | B | A | C |
| 150 | A | NT | NT | NT |
| 151 | B | NT | NT | NT |
| 152 | A | A | B | C |
| 153 | A | A | A | C |
| 154 | A | A | B | B |
| 155 | A | A | B | B |
| 157 | B | NT | NT | NT |
| 158 | B | NT | NT | NT |
| 159 | C | NT | NT | NT |
| 160 | B | NT | NT | NT |
| 161 | A | B | B | C |
| 162 | A | B | B | C |
| 163 | A | B | B | C |
| 164 | A | B | B | C |
| 165 | A | B | B | C |
| 166 | B | NT | NT | NT |
| 167 | A | A | A | C |
| 168 | A | A | A | C |
| 169 | A | A | A | C |
| 170 | A | A | A | B |
| 171 | A | NT | NT | NT |
| 172 | A | A | A | B |
| 173 | B | NT | NT | NT |
| 174 | B | NT | NT | NT |
| 175 | A | A | B | B |
| 176 | A | B | B | C |
| 177 | A | A | B | C |
| 178 | A | A | A | B |
| 179 | A | C | C | C |
| 180 | A | A | A | B |
| 181 | A | A | A | C |
| 182 | A | A | B | B |
| 183 | A | A | B | C |
| 184 | A | B | B | C |
| 185 | A | A | A | B |
| 186 | A | B | A | B |
| 187 | B | C | C | C |
| 188 | A | B | B | C |
| 189 | B | NT | NT | NT |
| 190 | A | B | B | C |
| 191 | A | NT | NT | NT |
| 192 | B | NT | NT | NT |
| 193 | A | A | B | C |
| 194 | C | NT | NT | NT |
| 195 | A | B | B | C |
| 196 | A | B | B | C |
| 197 | C | NT | NT | NT |
| 198 | A | NT | NT | NT |
| 199 | B | NT | NT | NT |
| 200 | A | NT | NT | NT |
| 201 | A | NT | NT | NT |
| 202 | B | B | B | C |
| 203 | B | NT | NT | NT |
| 204 | B | NT | NT | NT |
| 205 | C | NT | NT | NT |
| 206 | A | B | B | C |
| 207 | C | NT | NT | NT |
| 208 | B | NT | NT | NT |
| 209 | B | NT | NT | NT |
| 210 | B | NT | NT | NT |
| 211 | B | NT | NT | NT |
| 212 | B | NT | NT | NT |
| 213 | A | NT | NT | NT |
| 214 | A | A | B | C |
| 215 | A | A | B | C |
| 216 | A | A | A | C |
| 217 | A | A | A | C |
| 218 | A | B | B | C |
| 219 | A | A | B | C |
| 220 | A | B | B | C |
| 221 | B | NT | NT | NT |
| 222 | A | NT | NT | NT |
| 223 | A | A | B | C |
| 224 | C | NT | NT | NT |
| 225 | A | NT | NT | NT |
| 226 | A | NT | NT | NT |
| 227 | B | NT | NT | NT |
| 228 | A | B | B | C |
| 229 | A | NT | NT | NT |
| 230 | A | NT | NT | NT |
| 231 | B | B | C | C |
| 232 | B | C | C | C |
| 233 | A | B | B | NT |
| 234 | A | B | B | C |
| 235 | A | B | B | C |
| 236 | A | B | C | C |
| 237 | A | B | B | C |
| 238 | B | NT | NT | NT |
| 239 | B | NT | NT | NT |
| 240 | B | NT | NT | NT |
| 241 | A | B | B | C |
| 242 | A | B | B | C |
| 243 | B | NT | NT | NT |
| 244 | C | NT | NT | NT |
| 245 | C | NT | NT | NT |
| 246 | A | B | B | C |
| 247 | A | A | A | B |
| 248 | C | NT | NT | NT |
| 249 | B | NT | NT | NT |
| 250 | B | B | C | C |
| 251 | C | NT | NT | NT |
| 252 | B | B | C | C |
| 253 | B | NT | NT | NT |
| 254 | B | NT | NT | NT |
| 255 | A | B | B | C |
| 256 | B | NT | NT | NT |
| 257 | B | NT | NT | NT |
| 258 | B | NT | NT | NT |
| 259 | B | NT | NT | NT |
| 260 | C | NT | NT | NT |
| 261 | B | B | C | C |
| 262 | B | NT | NT | NT |
| 263 | A | NT | NT | NT |
| 264 | A | A | A | C |
| 265 | C | NT | NT | NT |
| 266 | C | NT | NT | NT |
| 267 | B | NT | NT | NT |
| 268 | A | B | C | C |
| 269 | A | B | B | C |
| 270 | A | A | B | C |
| 271 | A | A | A | A |
| 272 | A | A | B | C |
| 273 | A | B | B | C |
| 274 | A | A | A | C |
| 275 | A | B | B | B |
| 276 | B | NT | NT | NT |
| 277 | C | NT | NT | NT |
| 278 | B | NT | NT | NT |
| 279 | B | NT | NT | NT |
| 280 | B | NT | NT | NT |
| 281 | B | NT | NT | NT |
| 282 | A | A | A | C |
| 283 | A | B | B | B |
| 284 | B | NT | NT | NT |
| 285 | A | B | B | C |
| 286 | B | NT | NT | NT |
| 287 | A | B | B | B |
| 288 | A | B | B | C |
| 289 | B | C | C | C |
| 290 | A | B | B | C |
| 291 | B | B | C | C |
| 292 | C | NT | NT | NT |
| 293 | B | NT | NT | NT |
| 294 | A | A | A | C |
| 295 | A | A | B | C |
| 401 | A | A | A | C |
| 402 | A | A | A | B |
| 403 | A | A | B | C |
| 404 | A | B | B | C |
| 405 | A | A | B | C |
| 406 | A | B | B | C |
| 407 | B | C | C | C |
| 408 | A | A | B | C |
| 409 | A | A | A | B |
| 410 | A | A | B | C |
| 411 | A | A | A | C |
| 412 | A | A | A | C |
| 413 | A | A | B | C |
| 414 | A | A | A | C |
| 415 | A | B | B | B |
| 416 | A | B | B | C |
| 417 | A | A | B | B |
| 418 | B | NT | NT | NT |
| 419 | A | B | B | B |
| 420 | A | A | B | B |
| 421 | A | A | A | C |
| 422 | A | A | A | C |
| 423 | A | A | A | C |
| 424 | A | A | B | C |
| 425 | A | A | A | C |
| 427 | C | NT | NT | NT |
| 428 | A | A | A | C |
| 429 | A | B | B | C |
| 430 | A | A | A | C |
| 431 | A | A | B | C |
| 432 | A | A | A | C |
| 433 | A | NT | NT | NT |
| 434 | A | A | A | B |
| 435 | A | NT | NT | NT |
| 436 | B | B | B | C |
| 437 | A | B | B | C |
| 438 | >10 uM | NT | NT | NT |

TABLE 5b

| Additional Examples | Enzyme Inhibition |
|---|---|
| 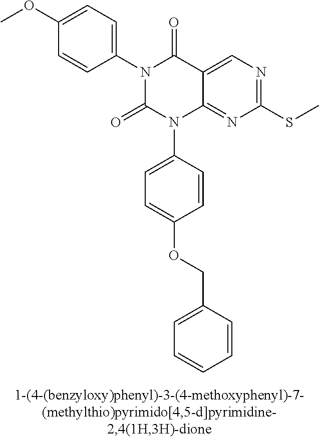 | D |
| 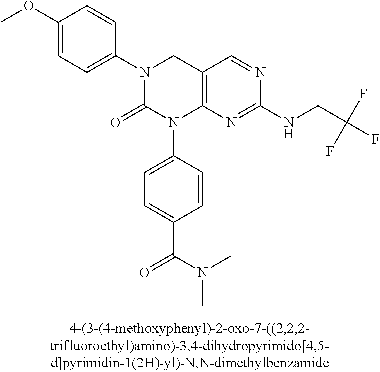 [SMILES: COc1ccc(N2Cc3cnc(NCC(F)(F)F)nc3N(c3ccc(C(=O)N(C)C)cc3)C2=O)cc1] | B |
| 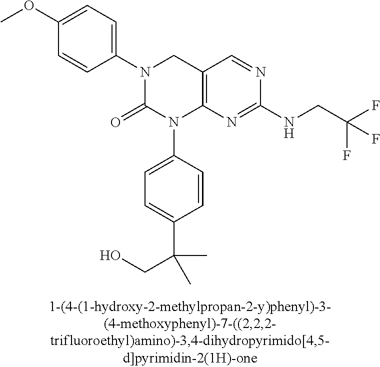 [SMILES: COc1ccc(N2Cc3cnc(NCC(F)(F)F)nc3N(c3ccc(C(C)(C)CO)cc3)C2=O)cc1] | B |
| 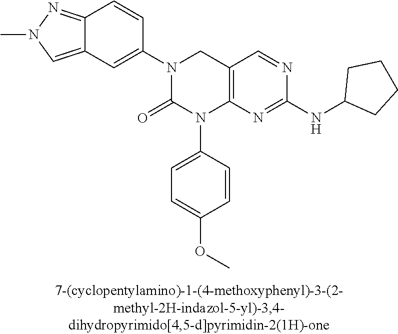 [SMILES: COc1ccc(N2C(=O)N(c3ccc4nn(C)cc4c3)Cc3cnc(NC4CCCC4)nc32)cc1] | B |
| 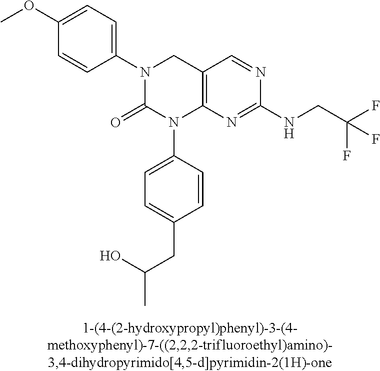 [SMILES: COc1ccc(N2Cc3cnc(NCC(F)(F)F)nc3N(c3ccc(CC(C)O)cc3)C2=O)cc1] | B |
| 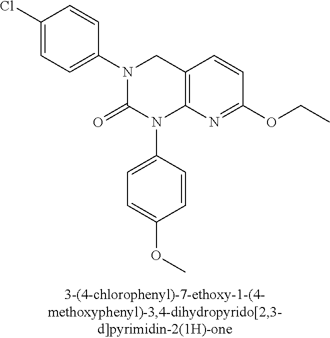 [SMILES: CCOc1ccc2c(n1)N(c1ccc(OC)cc1)C(=O)N(c1ccc(Cl)cc1)C2] | D |
| 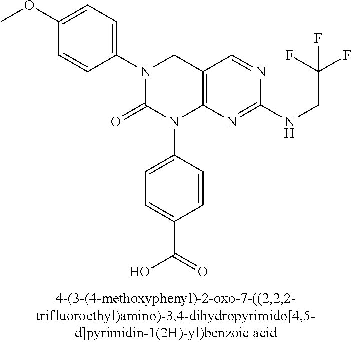 [SMILES: COc1ccc(N2Cc3cnc(NCC(F)(F)F)nc3N(c3ccc(C(=O)O)cc3)C2=O)cc1] | D |
| 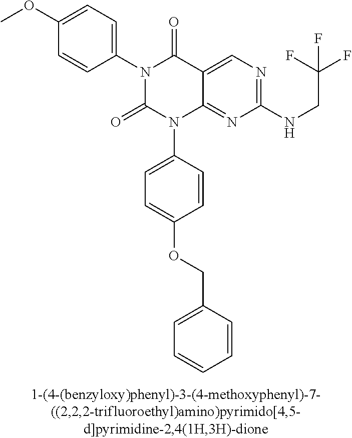 [SMILES: COc1ccc(-n2c(=O)c3cnc(NCC(F)(F)F)nc3n(-c3ccc(OCc4ccccc4)cc3)c2=O)cc1] | D |
| 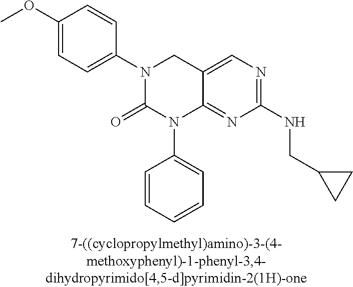 [SMILES: COc1ccc(N2Cc3cnc(NCC4CC4)nc3N(c3ccccc3)C2=O)cc1] | D |
| 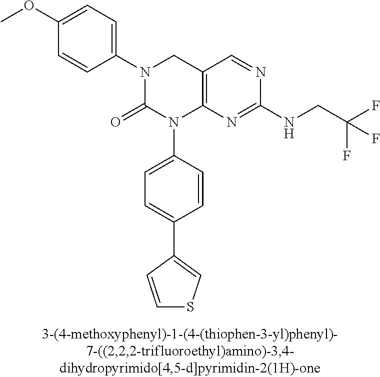 [SMILES: COc1ccc(N2Cc3cnc(NCC(F)(F)F)nc3N(c3ccc(-c4ccsc4)cc3)C2=O)cc1] | D |
| 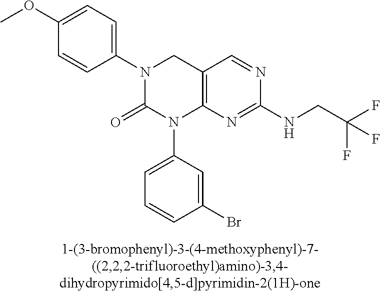 [SMILES: COc1ccc(N2Cc3cnc(NCC(F)(F)F)nc3N(c3cccc(Br)c3)C2=O)cc1] | D |
| 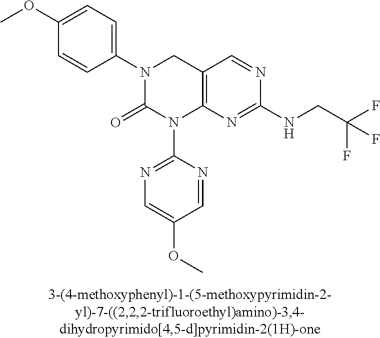 [SMILES: COc1ccc(N2Cc3cnc(NCC(F)(F)F)nc3N(c3ncc(OC)cn3)C2=O)cc1] | D |
| 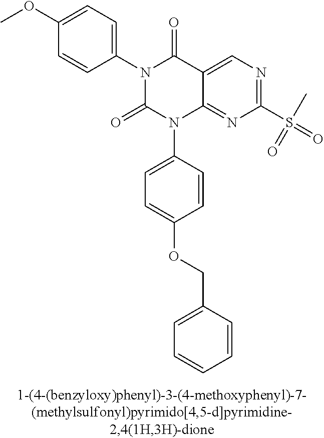 [SMILES: COc1ccc(-n2c(=O)c3cnc(S(C)(=O)=O)nc3n(-c3ccc(OCc4ccccc4)cc3)c2=O)cc1] | D |
| 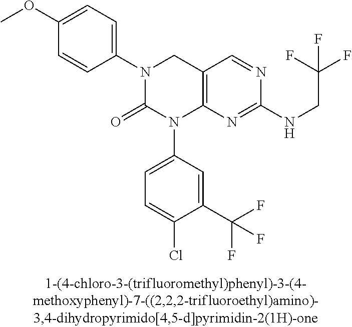 [SMILES: COc1ccc(N2Cc3cnc(NCC(F)(F)F)nc3N(c3ccc(Cl)c(C(F)(F)F)c3)C2=O)cc1] | D |
| 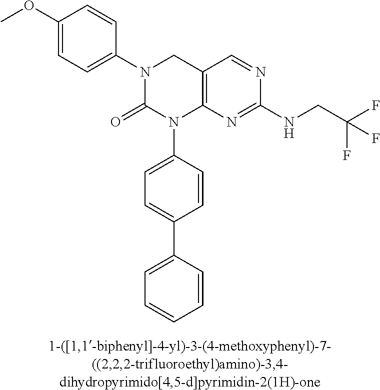 [SMILES: COc1ccc(N2Cc3cnc(NCC(F)(F)F)nc3N(c3ccc(-c4ccccc4)cc3)C2=O)cc1] | D |
| 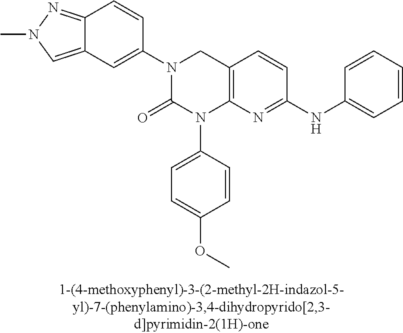 [SMILES: COc1ccc(N2C(=O)N(c3ccc4nn(C)cc4c3)Cc3ccc(Nc4ccccc4)nc32)cc1] | D |
| 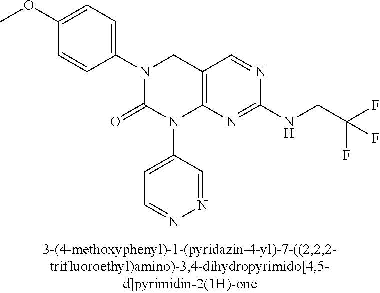 [SMILES: COc1ccc(N2Cc3cnc(NCC(F)(F)F)nc3N(c3ccnnc3)C2=O)cc1] | D |
| 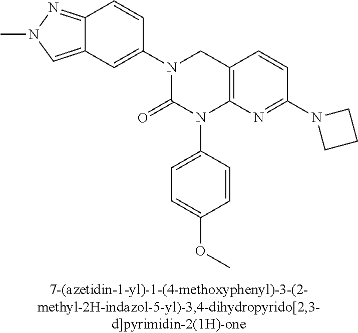 [SMILES: COc1ccc(N2C(=O)N(c3ccc4nn(C)cc4c3)Cc3ccc(N4CCC4)nc32)cc1] | D |
| 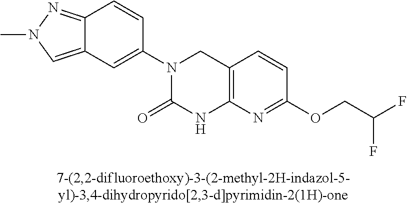 [SMILES: Cn1cc2cc(N3Cc4ccc(OCC(F)F)nc4NC3=O)ccc2n1] | D |
| 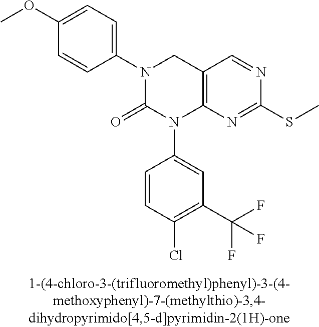 | D |
| 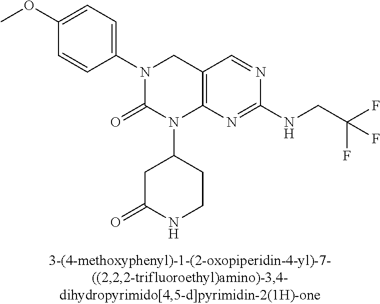 [SMILES: COc1ccc(N2Cc3cnc(NCC(F)(F)F)nc3N(C3CCNC(=O)C3)C2=O)cc1] | D |
| 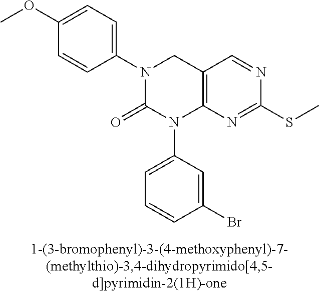 | D |
| 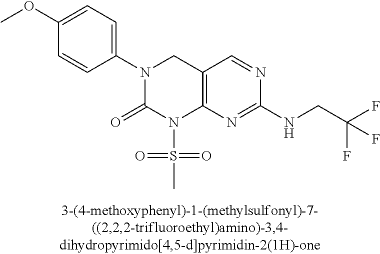 [SMILES: COc1ccc(N2Cc3cnc(NCC(F)(F)F)nc3N(S(C)(=O)=O)C2=O)cc1] | D |
| 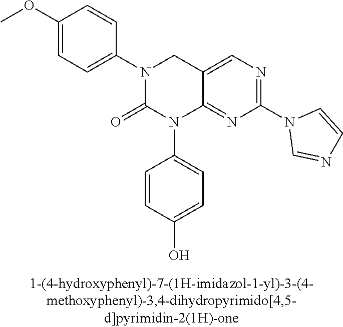 [SMILES: COc1ccc(N2Cc3cnc(-n4ccnc4)nc3N(c3ccc(O)cc3)C2=O)cc1] | D |

Table 6 below compares assay results for select pairs of compounds according to this disclosure.

TABLE 6

| | | Cell 72 h | 4 Day Relative |
|---|---|---|---|
| | | SAM | Growth |
| | | Inhibition | Inhibition |
| Example | Structure | (MTAP −/−) | (MTAP −/−) |
| 190 | 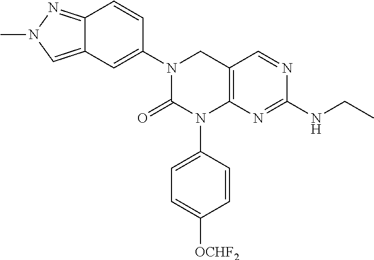 [SMILES: CCNc1ncc2c(n1)N(c1ccc(OC(F)F)cc1)C(=O)N(c1ccc3nn(C)cc3c1)C2] | B | B |
| 216 | 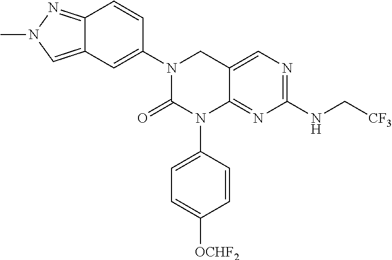 [SMILES: Cn1cc2cc(N3Cc4cnc(NCC(F)(F)F)nc4N(c4ccc(OC(F)F)cc4)C3=O)ccc2n1] | A | A |
| 269 | 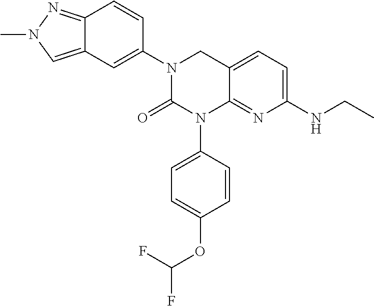 [SMILES: CCNc1ccc2c(n1)N(c1ccc(OC(F)F)cc1)C(=O)N(c1ccc3nn(C)cc3c1)C2] | B | B |
| 264 | 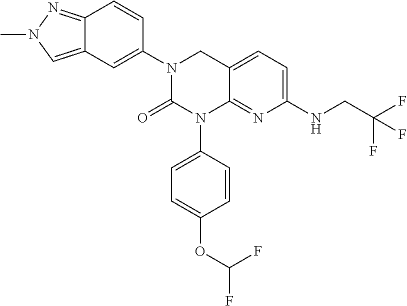 [SMILES: Cn1cc2cc(N3Cc4ccc(NCC(F)(F)F)nc4N(c4ccc(OC(F)F)cc4)C3=O)ccc2n1] | A | A |
| 274 | 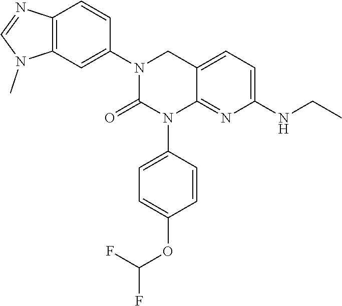 [SMILES: CCNc1ccc2c(n1)N(c1ccc(OC(F)F)cc1)C(=O)N(c1ccc3ncn(C)c3c1)C2] | A | A |
| 271 | 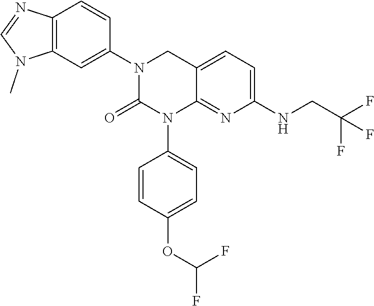 [SMILES: Cn1cnc2ccc(N3Cc4ccc(NCC(F)(F)F)nc4N(c4ccc(OC(F)F)cc4)C3=O)cc21] | A | A |
| 161 | 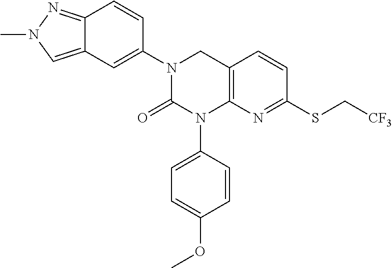 [SMILES: COc1ccc(N2C(=O)N(c3ccc4nn(C)cc4c3)Cc3ccc(SCC(F)(F)F)nc32)cc1] | B | B |
| 132 | 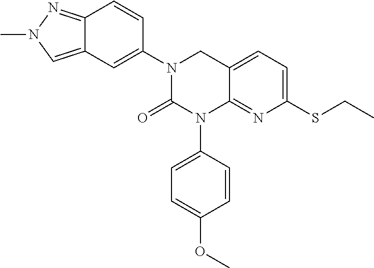 [SMILES: CCSc1ccc2c(n1)N(c1ccc(OC)cc1)C(=O)N(c1ccc3nn(C)cc3c1)C2] | B | B |

1. A compound according to Formula I. 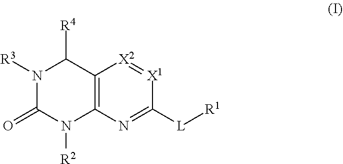
wherein:
X¹ is N or CR⁵;
X² is N or CR⁶, wherein X¹ and X² are not simultaneously N;
L is O, S, NR, or a bond;
R is H or C₁-C₆-alkyl;
R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein:
any alkyl in R¹ is straight or branched,
R¹ is optionally substituted by 1 to 6 halogen; and
when X¹ is N, X² is CR⁶, L is NR or S, R is H, and R¹ is C₁-C₆-alkyl, then R¹ is substituted by 1 to 6 halogen;
or when L is NR, then R and R¹ can be taken together in combination with L to form a 3- to 6-membered heterocycloalkyl (wherein 1-4 ring members are, independently, N, O, or S) optionally substituted by one or more of R^{A};
R² and R³ are independently selected from the group consisting of C₆-C₁₀-aryl, C₃-C₆-carbocyclyl, 5- to 10-membered heteroaryl (wherein 1-4 heteroaryl members are, independently, N, O, or S), and 3- to 14-membered heterocycloalkyl (wherein 1-4 heterocycloalkyl members are, independently, N, O, or S),
wherein R² and R³ are independently and optionally substituted by one or more substituents that are selected from the group consisting of R^{A}, OR^{A}, halogen, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN;
R⁴ is selected from the group consisting of H, C₁-C₆-alkyl, C₁-C₆-alkoxy, C₂-C₆-alkenyl, C₂-C₆-alkynyl, halogen, oxo, —CN, and —NR^{C}R^{D};
R⁵ is selected from the group consisting of H, C₁-C₆-alkyl, C₁-C₆-alkoxy, C₂-C₆-alkenyl, C₂-C₆-alkynyl, halogen, —CN, and —NR^{C}R^{D};
R⁶ is selected from the group consisting of H, C₁-C₆-alkyl (optionally substituted by one or more halogen), —O(C₁-C₆-alkyl) (optionally substituted by one or more halogen), —OH, halogen, —CN, —(C₁-C₆-alkyl)NR^{A}R^{B}, and —NR^{A}R^{B};
R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl and —(C₁-C₆-alkyl)-(3- to 14-membered heterocycloalkyl) (wherein 1-4 heterocycloalkyl members are, independently, N, O, or S), and 5- to 10-membered heteroaryl (wherein 1-4 heteroaryl members are, independently, N, O, or S);
wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of deuterium, hydroxy, halogen, —NR′₂ (wherein each R′ is independently selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are, independently, N, O, or S), and 5- to 10-membered heteroaryl (wherein 1-4 heteroaryl members are, independently, N, O, and S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl-C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are, independently, N, O, or S), and —O(C₆-C₁₄-aryl),
wherein each alkyl, alkenyl, aryl, and heterocycloalkyl is optionally substituted with one or more substituents selected from the group consisting of hydroxy, —OC₁-C₆-alkyl, halogen, —NH₂, —(C₁-C₆-alkyl)NH₂, —C(O)OH, CN, and oxo;
R^{C} and R^{D} are each, independently, H or C₁-C₆-alkyl;
or a pharmaceutically acceptable salt thereof.

2. A compound according to Formula II. 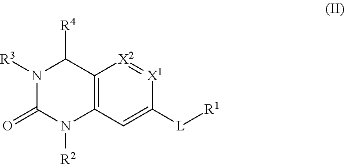
wherein:
X¹ is N and X² is CR⁶, or X¹ is CR⁵ and X² is CR⁶, or X¹ and X² are both N, or X¹ is CR⁵ and X² is N;
L is O, S, NR, or a bond;
R is H or C₁-C₆-alkyl;
R¹ is selected from the group consisting of C₁-C₆-alkyl, C₂-C₆-alkenyl, C₃-C₆-carbocyclyl, —(C₁-C₆-alkyl)(C₃-C₆-carbocyclyl), and —(C₁-C₆-alkyl)(C₃-C₆-cycloalkenyl) wherein:
any alkyl in R¹ is straight or branched,
R¹ is optionally substituted by 1 to 6 halogen;
or when L is NR, then R and R¹ can be taken together in combination with L to form a 3- to 6-membered heterocycloalkyl (wherein 1-4 ring members are, independently, N, O, or S) optionally substituted by one or more of R^{A};
R² and R³ are independently selected from the group consisting of C₆-C₁₀-aryl, C₃-C₆-carbocyclyl, 5- to 10-membered heteroaryl (wherein 1-4 heteroaryl members are, independently, N, O, or S), and 3- to 14-membered heterocycloalkyl (wherein 1-4 heterocycloalkyl members are, independently, N, O, or S),
wherein R² and R³ are independently and optionally substituted by one or more substituents that are selected from the group consisting of R^{A}, OR^{A}, halogen, —N═N—R^{A}, —NR^{A}R^{B}, —(C₁-C₆-alkyl)NR^{A}R^{B}, —C(O)OR^{A}, —C(O)NR^{A}R^{B}, —OC(O)R^{A}, and —CN;
R⁴ is selected from the group consisting of H, C₁-C₆-alkyl, C₁-C₆-alkoxy, C₂-C₆-alkenyl, C₂-C₆-alkynyl, halogen, oxo, —CN, and —NR^{C}R^{D};
R⁵ is selected from the group consisting of H, C₁-C₆-alkyl, C₁-C₆-alkoxy, C₂-C₆-alkenyl, C₂-C₆-alkynyl, halogen, —CN, and —NR^{C}R^{D};
R⁶ is selected from the group consisting of H, C₁-C₆-alkyl (optionally substituted by one or more halogen), —O(C₁-C₆-alkyl) (optionally substituted by one or more halogen), —OH, halogen, —CN, —(C₁-C₆-alkyl)NR^{A}R^{B}, and —NR^{A}R^{B};
R^{A} and R^{B} are independently selected from the group consisting of H, —CN, -hydroxy, oxo, C₁-C₆-alkyl, C₁-C₆-alkoxy, C₂-C₆-alkenyl, C₂-C₆-alkynyl, —NH₂, —S(O)₀₋₂—(C₁-C₆-alkyl), —S(O)₀₋₂—(C₆-C₁₀-aryl), —C(O)(C₁-C₆-alkyl), —C(O)(C₃-C₁₄-carbocyclyl), —C₃-C₁₄-carbocyclyl, —(C₁-C₆-alkyl)(C₃-C₁₄-carbocyclyl), C₆-C₁₀-aryl, 3- to 14-membered heterocycloalkyl (wherein 1-4 heterocycloalkyl members are, independently, N, O, or S), —(C₁-C₆-alkyl)-(3- to 14-membered heterocycloalkyl) (wherein 1-4 heterocycloalkyl members are, independently, N, O, or S), and 5- to 10-membered heteroaryl (wherein 1-4 heteroaryl members are, independently, N, O, or S);
wherein each alkyl, alkoxy, alkenyl, alkynyl, aryl, carbocyclyl, heterocycloalkyl, and heteroaryl moiety of R^{A} and R^{B} is optionally substituted with one or more substituents selected from the group consisting of hydroxy, halogen, —NR′₂ (wherein each R′ is, independently, selected from the group consisting of C₁-C₆-alkyl, C₂-C₆-alkenyl, C₂-C₆-alkynyl, C₆-C₁₀-aryl, 3- to 14-membered heterocycloalkyl (wherein 1-4 ring members are, independently, N, O, or S), —(C₁-C₆-alkyl)-(3- to 14-membered heterocycloalkyl) (wherein 1-4 ring members are, independently, N, O, or S), and 5- to 10-membered heteroaryl (wherein 1-4 heteroaryl members are, independently, N, O, or S)), —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are, independently, N, O, or S), and —O(C₆-C₁₄-aryl),
wherein each alkyl, alkenyl, aryl, and heterocycloalkyl in R^{A} and R^{B} is optionally substituted with one or more substituents selected from the group consisting of hydroxy, —OC₁-C₆-alkyl, halogen, —NH₂, —(C₁-C₆-alkyl)NH₂, —C(O)OH, CN, and oxo,
R^{C} and R^{D} are each independently selected from H and C₁-C₆-alkyl;
or a pharmaceutically acceptable salt thereof.

3. The compound according to claim 1, wherein X¹ is N and X² is CR⁶.

4. The compound according to claim 1, wherein X¹ is CR⁵ and X² is CR⁶.

5. The compound according to claim 1, wherein X¹ is CR⁵ and X² is N.

6. The compound according to claim 2, wherein X¹ is CR⁵ and X² is CR⁶.

7. The compound according to claim 2, wherein X¹ is N and X² is CR⁶.

8. The compound according to claim 2, wherein X¹ and X² are both N.

9. The compound according to claim 2, wherein X¹ is CR⁵ and X² is CR⁶.

10. The compound according to claim 1, wherein each of R⁴ and R⁵ (when present) is, independently, H or C₁-C₆-alkyl, and R⁶ (when present) is selected from the group consisting of H, C₁-C₆-alkyl optionally substituted by one or more halogen, C₁-C₆-alkoxy, —(C₁-C₆-alkyl)NR^{A}R^{B}, and —NR^{A}R^{B} (wherein R^{A} and R^{B} are, independently, H or C₁-C₆-alkyl).

11. The compound according to claim 1, wherein at least one of R⁴, R⁵, and R⁶ (when present) is H.

12. The compound according to claim 1, wherein R⁴ is H.

13. The compound according to claim 1, wherein R⁵ is H.

14. The compound according to claim 1, wherein R⁶ is H.

15. The compound according to claim 1, wherein each of R⁴, R⁵, and R⁶ (when present) is H.

16. The compound according to claim 1, wherein R² is optionally substituted C₆-C₁₀-aryl or optionally substituted 5- to 10-membered heteroaryl (wherein 1-4 heteroaryl members are, independently, N, O, or S).

17. The compound according to claim 16, wherein R² is optionally substituted C₆-C₁₀-aryl.

18. The compound according to claim 17, wherein R² is optionally substituted phenyl.

19. The compound according to claim 16, wherein R² is optionally substituted 5- to 10-membered heteroaryl, and wherein 1 heteroaryl ring member is N.

20. The compound according to claim 19, wherein R² is optionally substituted pyridyl.

21. The compound according to claim 1, wherein R³ is optionally substituted 3- to 14-membered heterocycloalkyl (wherein 1-4 heterocycloalkyl members are, independently, N, O, or S) or optionally substituted 5- to 10-membered heteroaryl (wherein 1-4 heteroaryl members are, independently, N, O, or S).

22. The compound according to claim 21, wherein R³ is selected from the group consisting of benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl, any of which is optionally substituted.

23. The compound according to claim 1, wherein R³ is optionally substituted C₆-C₁₀-aryl.

24. The compound according to claim 23, wherein R³ is optionally substituted phenyl.

25. The compound according to claim 1, wherein R² is optionally substituted phenyl and R³ is optionally substituted 3- to 14-membered heterocycloalkyl (wherein 1-4 heterocycloalkyl members are, independently, N, O, or S) or optionally substituted 5- to 10-membered heteroaryl(wherein 1-4 heteroaryl members are, independently, N, O, or S).

26. The compound according to claim 1, wherein L is O or NR.

27. The compound according to claim 26, wherein R¹ is optionally substituted C₁-C₆-alkyl or optionally substituted C₃-C₆-carbocyclyl.

28. The compound according to claim 26, wherein R¹ is C₁-C₃-alkyl that is optionally substituted by 1-3 F.

29. The compound according to claim 1, wherein:
L is O or NR and R is H;
R¹ is C₁-C₃-alkyl that is optionally substituted by 1 to 3 F;
R² is optionally substituted 3- to 14-membered heterocycloalkyl (wherein 1 heterocycloalkyl member is N) or optionally substituted 5- to 10-membered heteroaryl (wherein 1 heteroaryl member is N) or optionally substituted C₆-C₁₀-aryl;
R³ is optionally substituted 3- to 14-membered heterocycloalkyl (wherein 1 to 3 heterocycloalkyl members are, independently, N, O, or S), optionally substituted 5- to 10-membered heteroaryl (wherein 1 to 3 heteroaryl members are, independently, N, O, or S), or optionally substituted C₆-C₁₀-aryl; and
each of R⁴, R⁵, and R⁶ (when present) is H.

30. The compound according to claim 29, wherein L is NR.

31. The compound according to claim 29, wherein
R² is optionally substituted phenyl; and
R³ is an optionally substituted 5- to 10-membered heteroaryl (wherein 1 to 3 heteroaryl members are, independently, N, O, or S).

32. The compound according to claim 29, wherein
R² is an optionally substituted 5- to 10-membered heteroaryl (wherein 1 to 3 heteroaryl members are, independently, N, O, or S); and
R³ is optionally substituted phenyl.

33. The compound according to claim 31, wherein R³ is selected from the group consisting of optionally substituted benzothiazolyl, benzoisothiazolyl, benzoxazolyl, pyridinyl, pyridinonyl, pyridazinyl, benzimidazolyl, benzotriazolyl, indazolyl, quinoxalinyl, quinolinyl, quinazolinyl, imidazopyridinyl, pyrazolopyridinyl, triazolopyridinyl, cinnolinyl, isoxazolyl, pyrazolyl, benzofuranyl, dihydrobenzofuranyl, dihydrobenzodioxinyl, and tetrahydrobenzodioxinyl, any of which is optionally substituted.

34. The compound according to claim 29, wherein R² and R³ independently are optionally substituted phenyl.

35. The compound according to claim 1 or a pharmaceutically acceptable salt thereof, wherein the compound is:

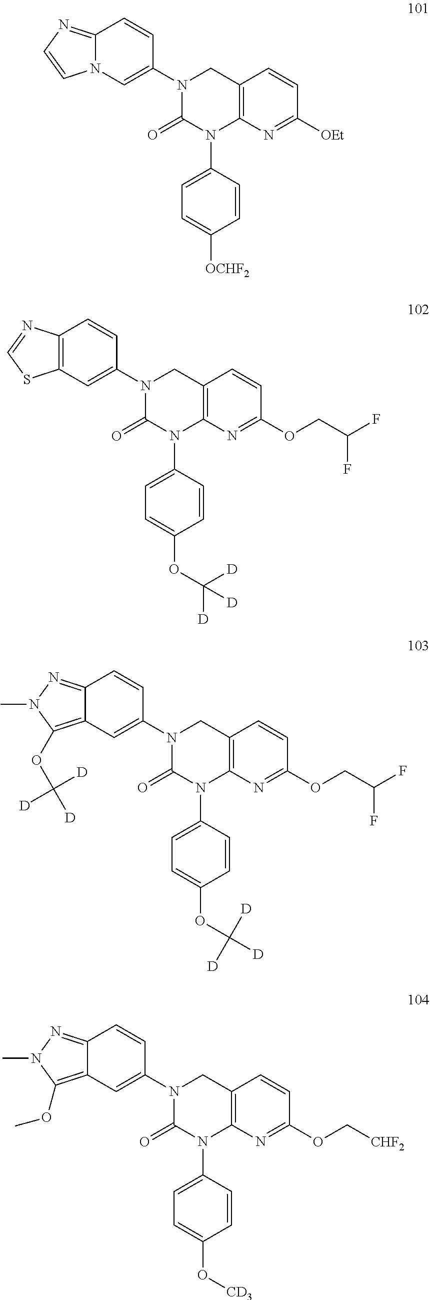 [SMILES: CCOc1ccc2c(n1)N(c1ccc(OC(F)F)cc1)C(=O)N(c1ccc3nccn3c1)C2 ; [2H]C([2H])([2H])Oc1ccc(N2C(=O)N(c3ccc4ncsc4c3)Cc3ccc(OCC(F)F)nc32)cc1 ; [2H]C([2H])([2H])Oc1ccc(N2C(=O)N(c3ccc4nn(C)c(OC([2H])([2H])[2H])c4c3)Cc3ccc(OCC(F)F)nc32)cc1 ; [2H]C([2H])([2H])Oc1ccc(N2C(=O)N(c3ccc4nn(C)c(OC)c4c3)Cc3ccc(OCC(F)F)nc32)cc1]

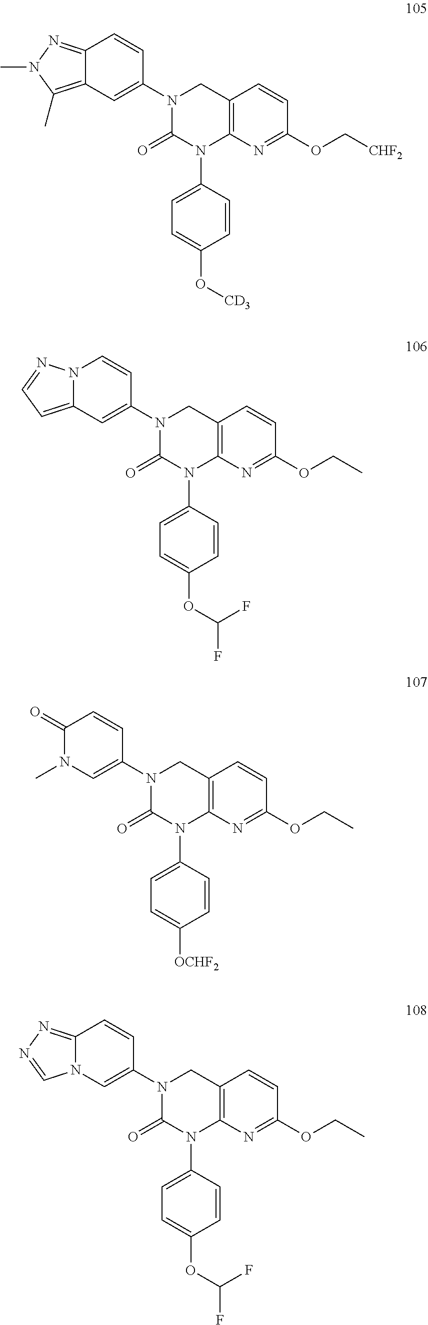

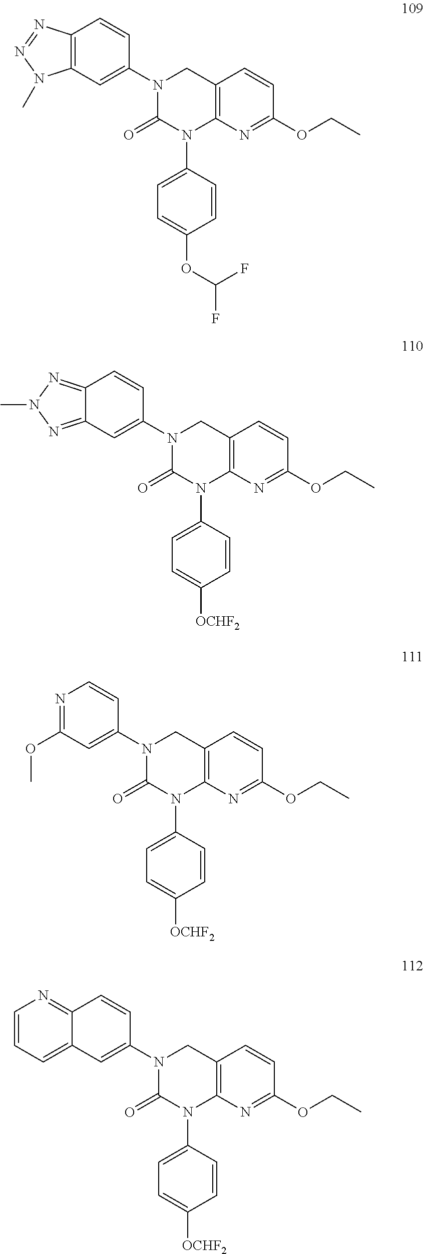

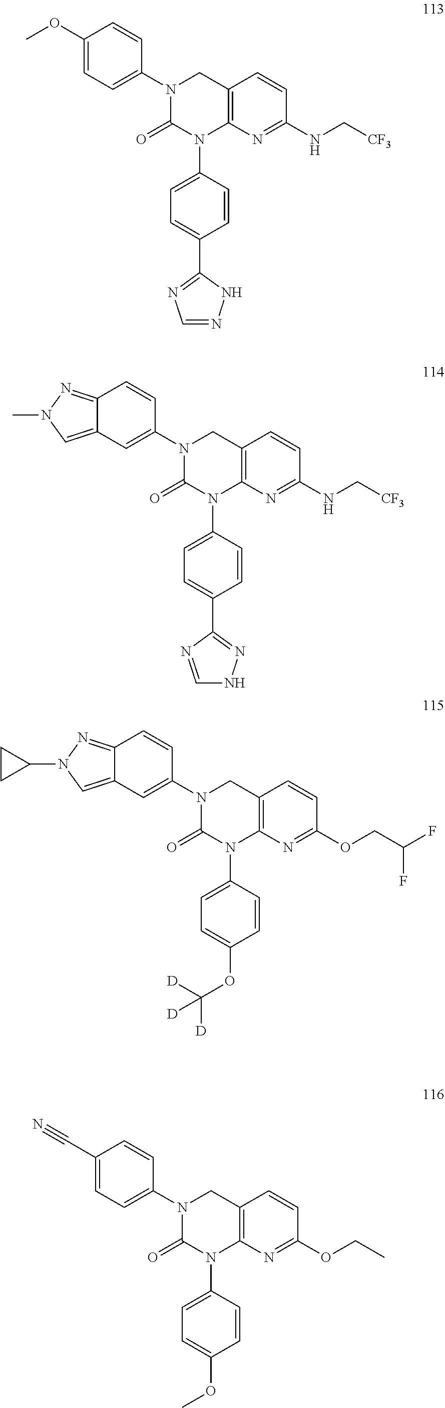 [SMILES: COc1ccc(N2Cc3ccc(NCC(F)(F)F)nc3N(c3ccc(-c4ncn[nH]4)cc3)C2=O)cc1 ; Cn1cc2cc(N3Cc4ccc(NCC(F)(F)F)nc4N(c4ccc(-c5nc[nH]n5)cc4)C3=O)ccc2n1 ; [2H]C([2H])([2H])Oc1ccc(N2C(=O)N(c3ccc4nn(C5CC5)cc4c3)Cc3ccc(OCC(F)F)nc32)cc1 ; CCOc1ccc2c(n1)N(c1ccc(OC)cc1)C(=O)N(c1ccc(C#N)cc1)C2]

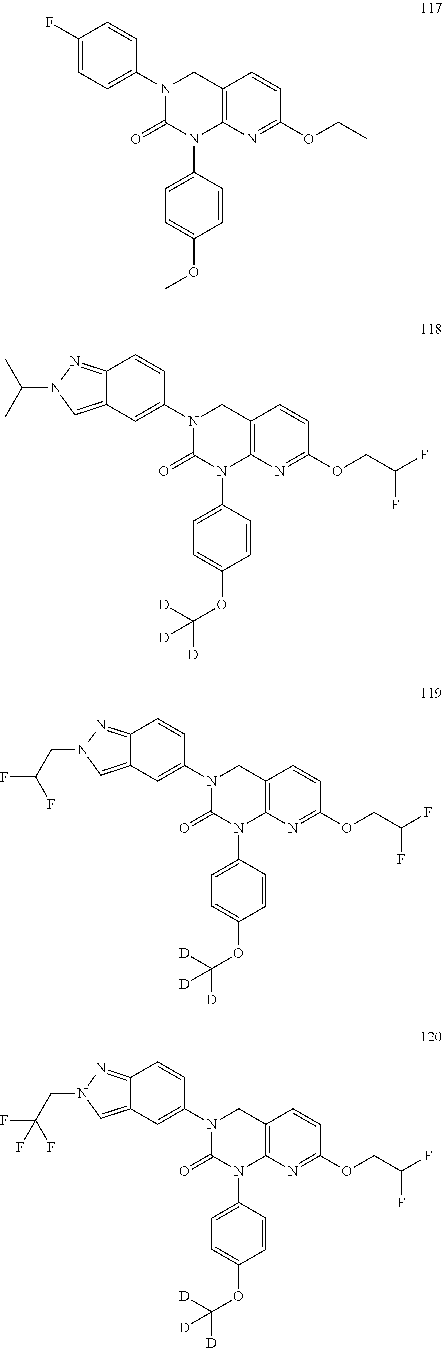

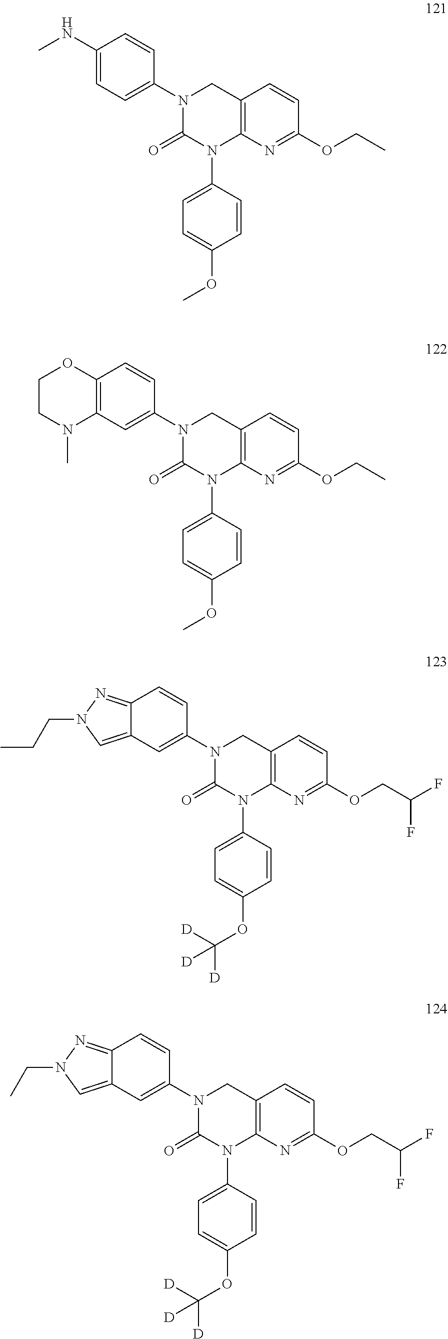

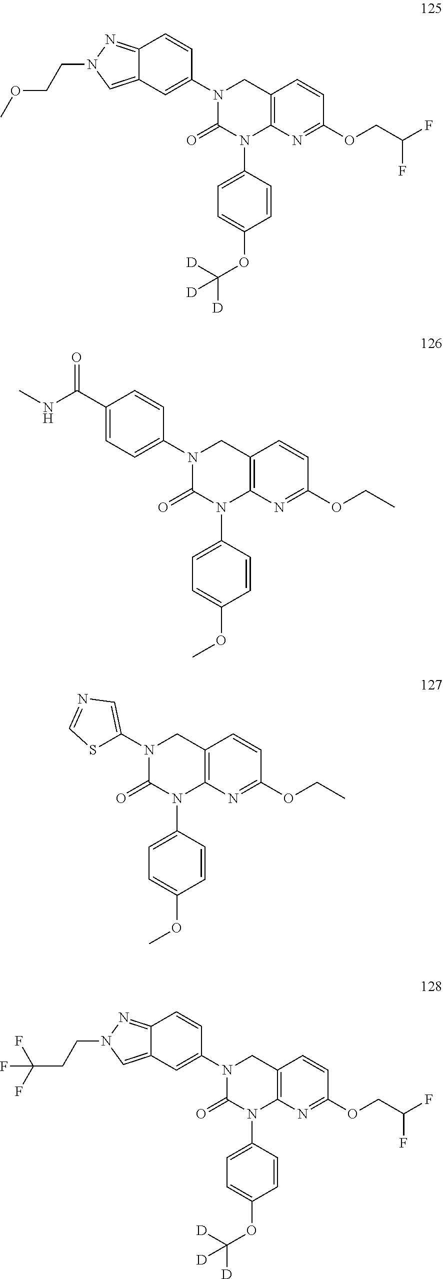 [SMILES: [2H]C([2H])([2H])Oc1ccc(N2C(=O)N(c3ccc4nn(CCOC)cc4c3)Cc3ccc(OCC(F)F)nc32)cc1 ; CCOc1ccc2c(n1)N(c1ccc(OC)cc1)C(=O)N(c1ccc(C(=O)NC)cc1)C2 ; CCOc1ccc2c(n1)N(c1ccc(OC)cc1)C(=O)N(c1cncs1)C2 ; [2H]C([2H])([2H])Oc1ccc(N2C(=O)N(c3ccc4nn(CCC(F)(F)F)cc4c3)Cc3ccc(OCC(F)F)nc32)cc1]

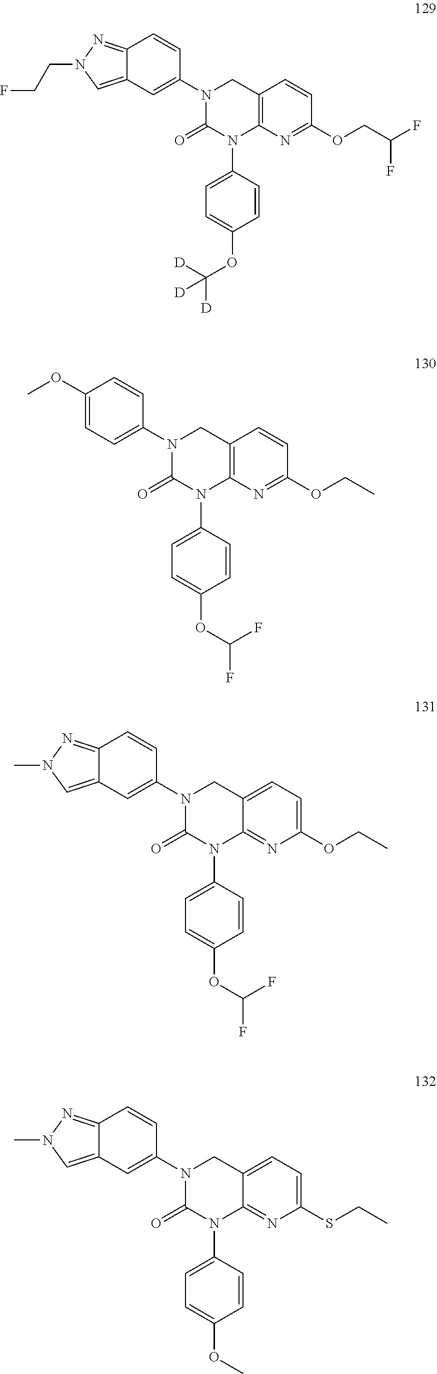

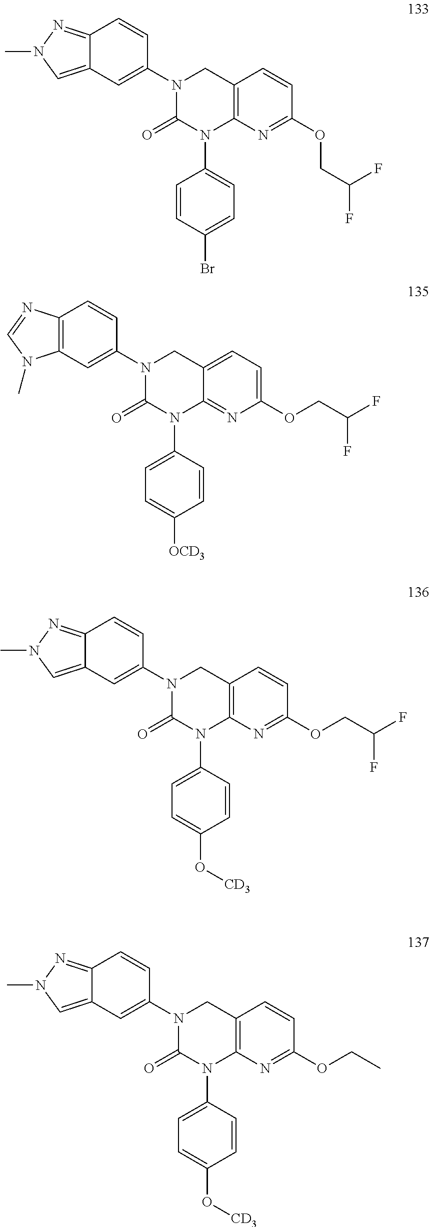

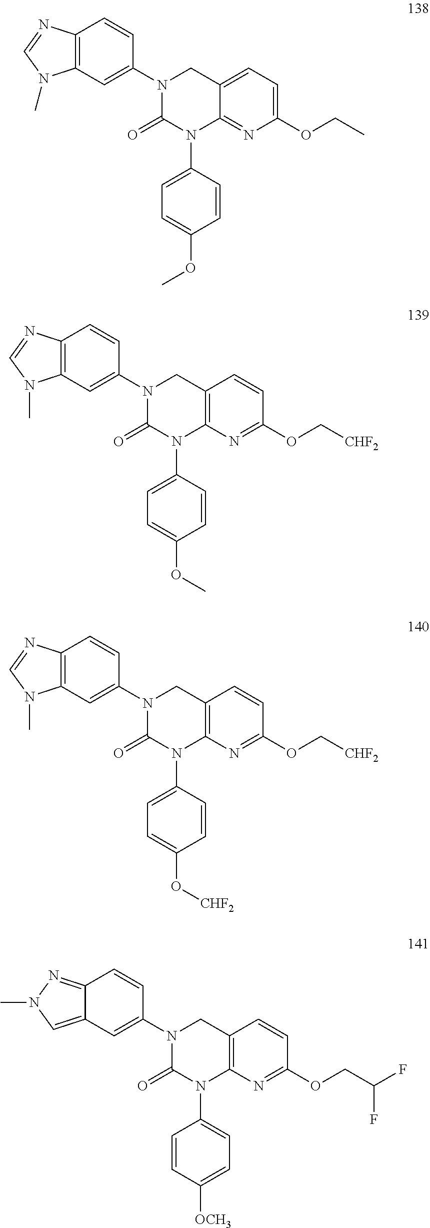

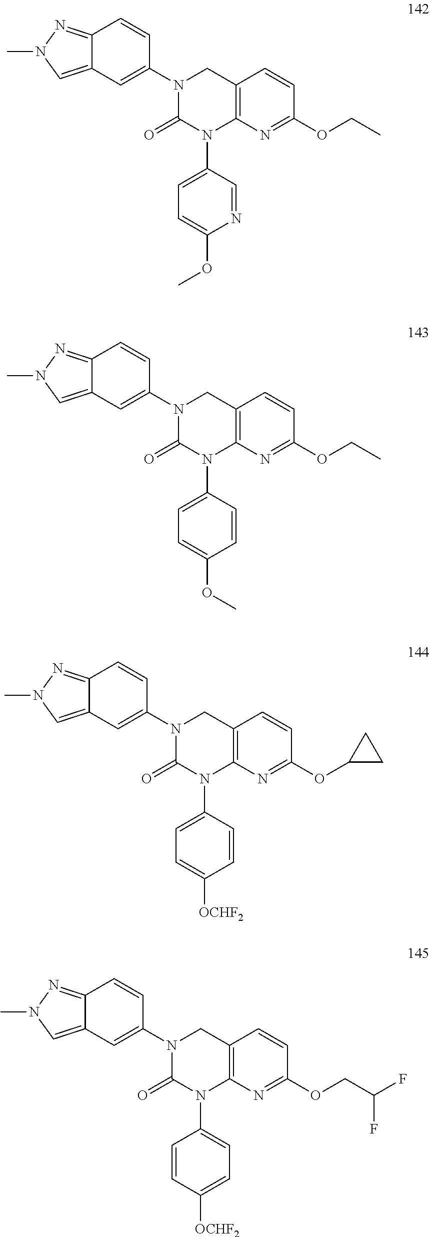

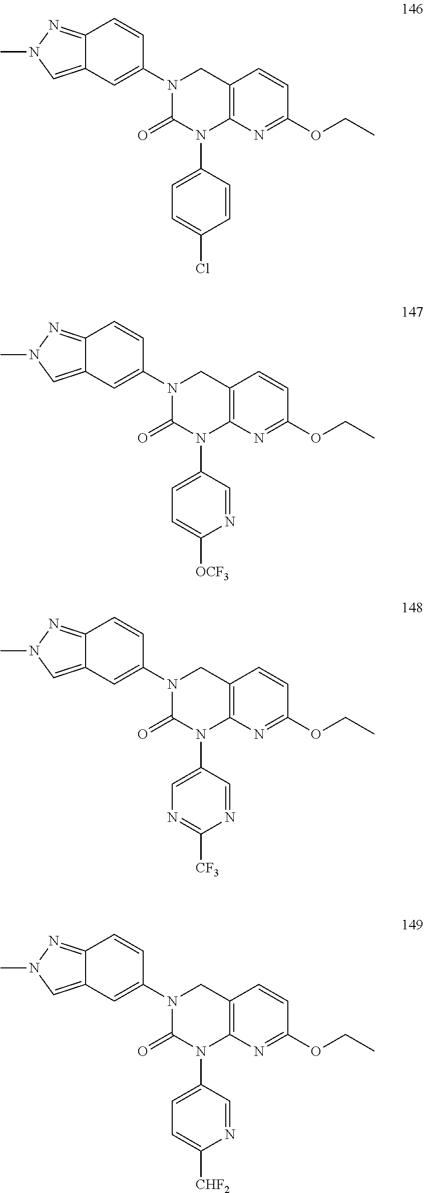

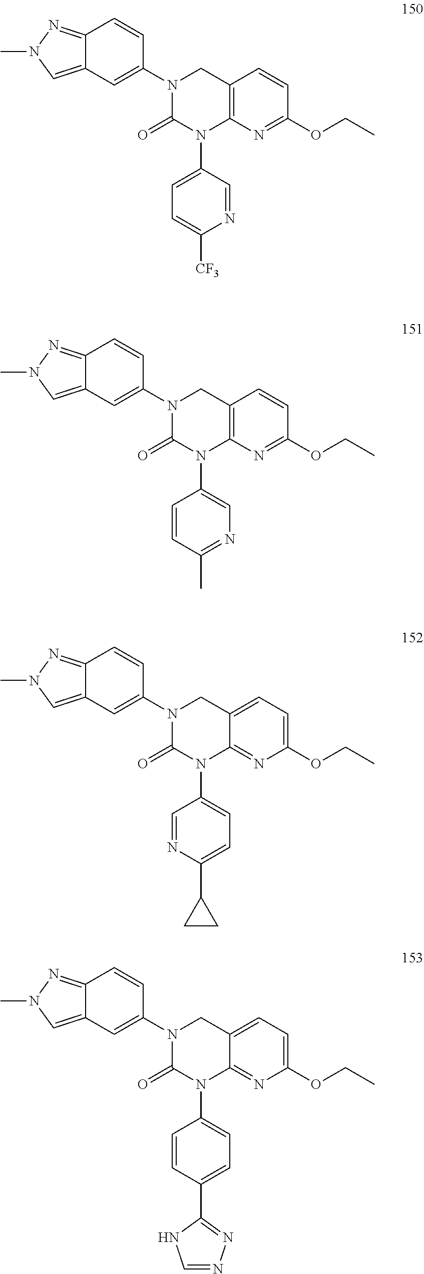

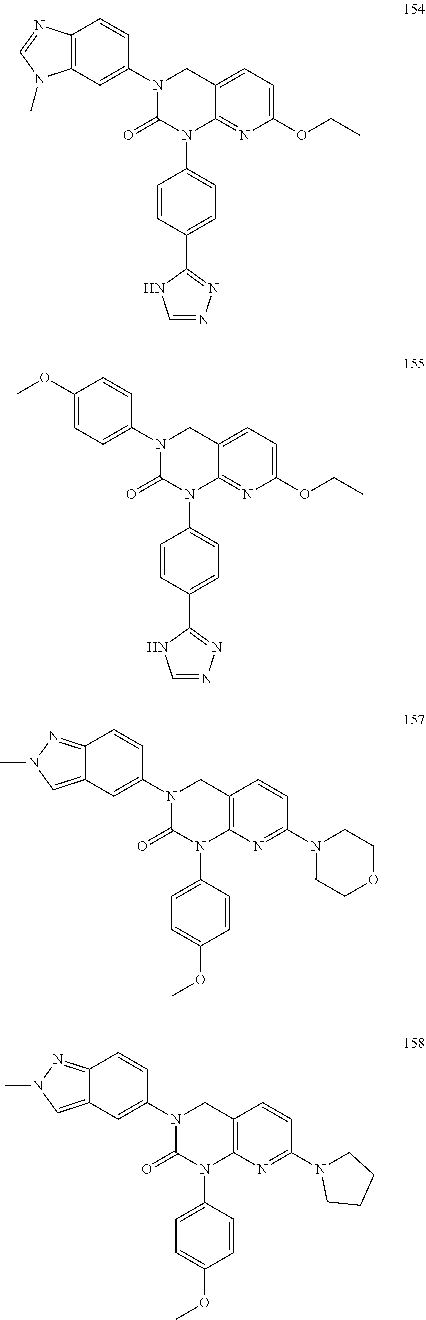

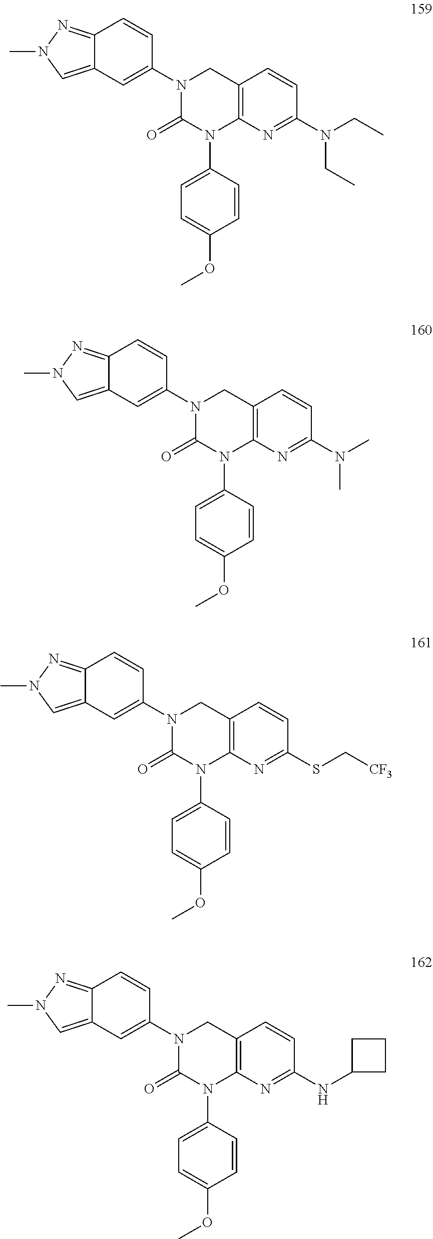

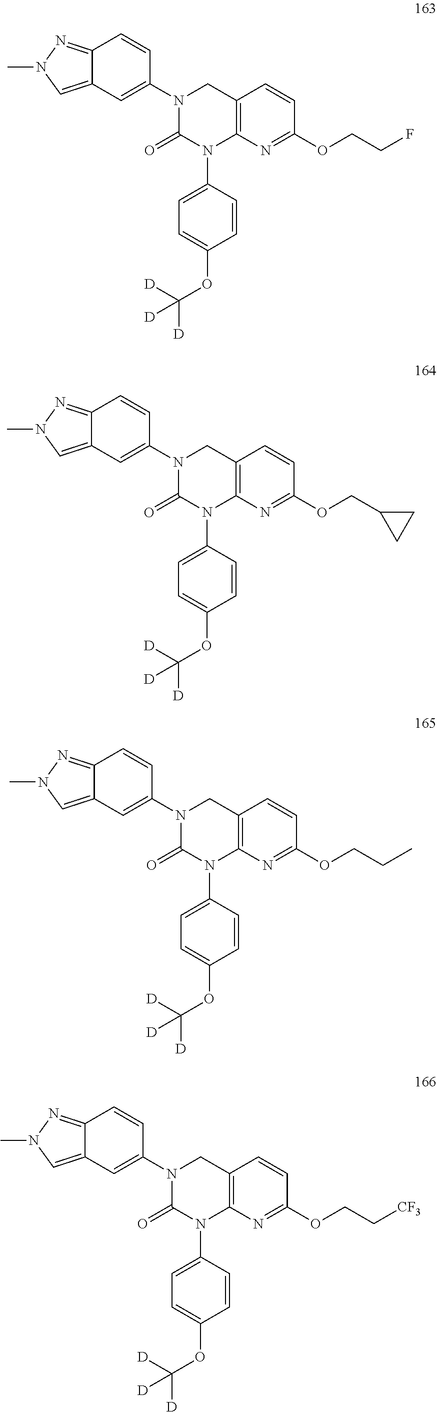

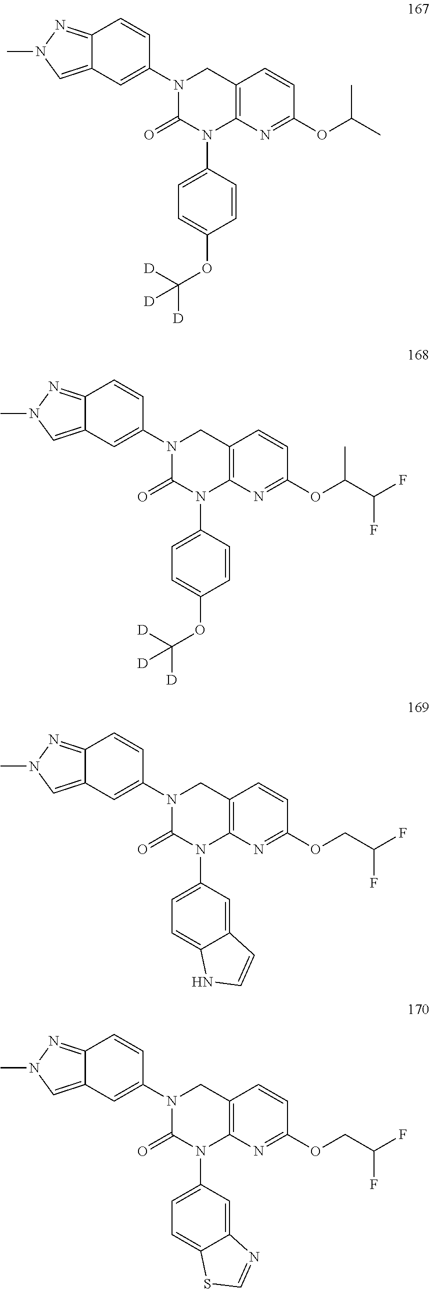

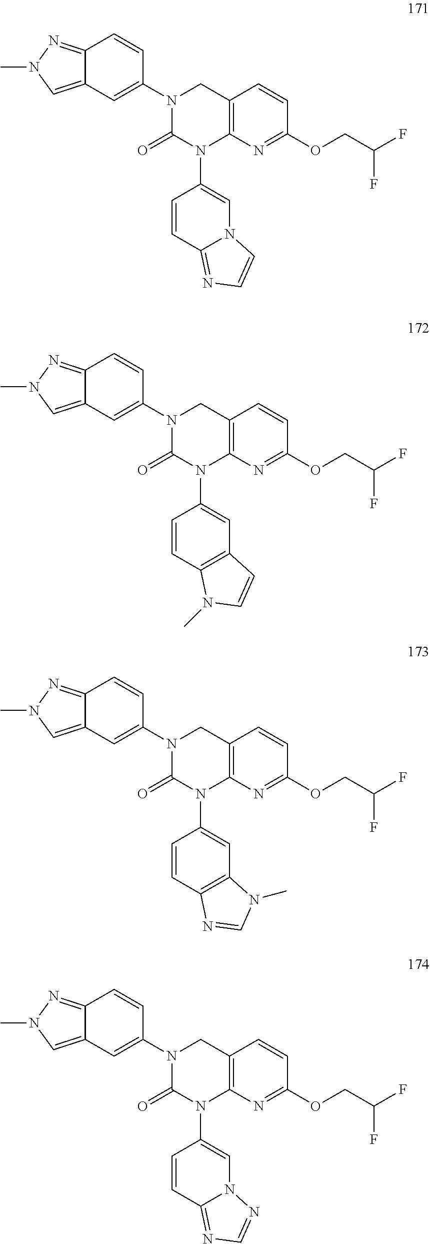 [SMILES: Cn1cc2cc(N3Cc4ccc(OCC(F)F)nc4N(c4ccc5nccn5c4)C3=O)ccc2n1 ; Cn1cc2cc(N3Cc4ccc(OCC(F)F)nc4N(c4ccc5c(ccn5C)c4)C3=O)ccc2n1 ; Cn1cc2cc(N3Cc4ccc(OCC(F)F)nc4N(c4ccc5ncn(C)c5c4)C3=O)ccc2n1 ; Cn1cc2cc(N3Cc4ccc(OCC(F)F)nc4N(c4ccc5ncnn5c4)C3=O)ccc2n1]

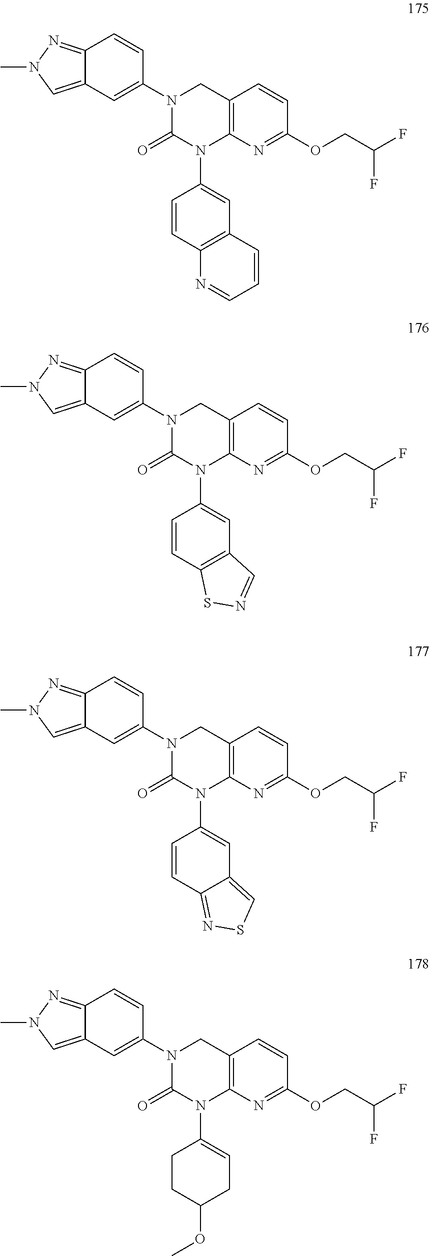 [SMILES: Cn1cc2cc(N3Cc4ccc(OCC(F)F)nc4N(c4ccc5ncccc5c4)C3=O)ccc2n1 ; Cn1cc2cc(N3Cc4ccc(OCC(F)F)nc4N(c4ccc5sncc5c4)C3=O)ccc2n1 ; Cn1cc2cc(N3Cc4ccc(OCC(F)F)nc4N(c4ccc5nscc5c4)C3=O)ccc2n1 ; COC1CC=C(N2C(=O)N(c3ccc4nn(C)cc4c3)Cc3ccc(OCC(F)F)nc32)CC1]

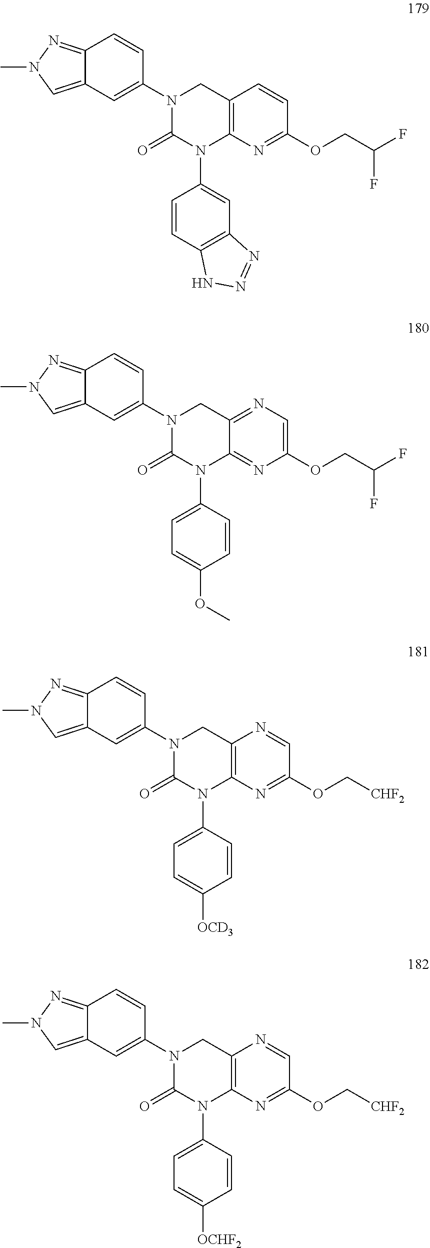

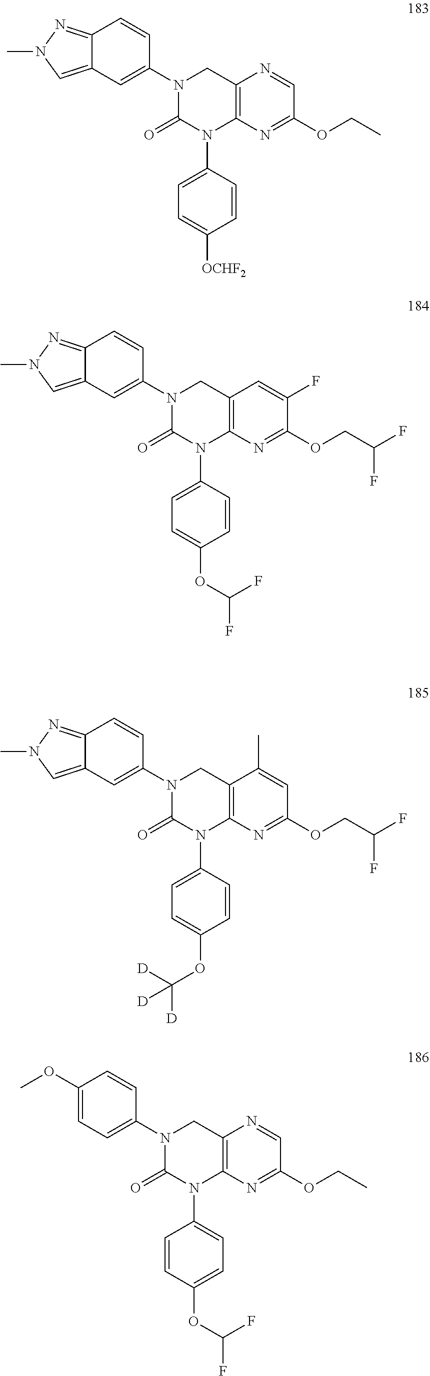

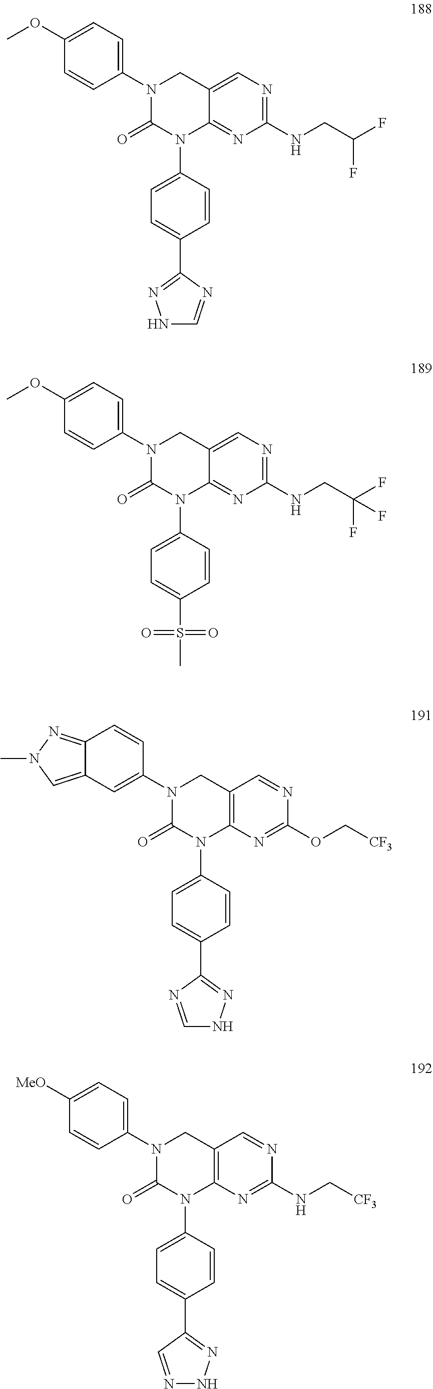 [SMILES: COc1ccc(N2Cc3cnc(NCC(F)F)nc3N(c3ccc(-c4nc[nH]n4)cc3)C2=O)cc1 ; COc1ccc(N2Cc3cnc(NCC(F)(F)F)nc3N(c3ccc(S(C)(=O)=O)cc3)C2=O)cc1 ; Cn1cc2cc(N3Cc4cnc(OCC(F)(F)F)nc4N(c4ccc(-c5nc[nH]n5)cc4)C3=O)ccc2n1 ; COc1ccc(N2Cc3cnc(NCC(F)(F)F)nc3N(c3ccc(-c4cn[nH]n4)cc3)C2=O)cc1]

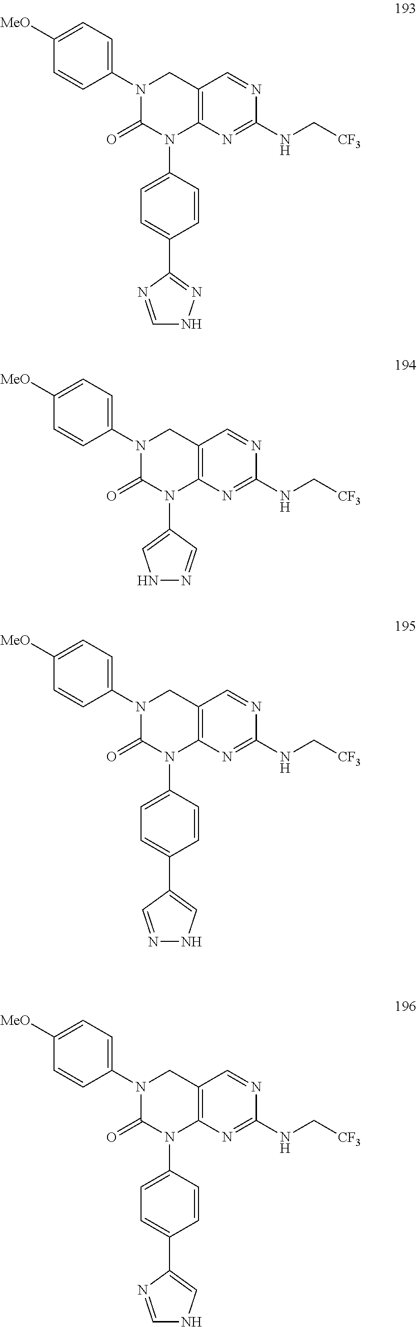 [SMILES: COc1ccc(N2Cc3cnc(NCC(F)(F)F)nc3N(c3ccc(-c4nc[nH]n4)cc3)C2=O)cc1 ; COc1ccc(N2Cc3cnc(NCC(F)(F)F)nc3N(c3cn[nH]c3)C2=O)cc1 ; COc1ccc(N2Cc3cnc(NCC(F)(F)F)nc3N(c3ccc(-c4cn[nH]c4)cc3)C2=O)cc1 ; COc1ccc(N2Cc3cnc(NCC(F)(F)F)nc3N(c3ccc(-c4c[nH]cn4)cc3)C2=O)cc1]

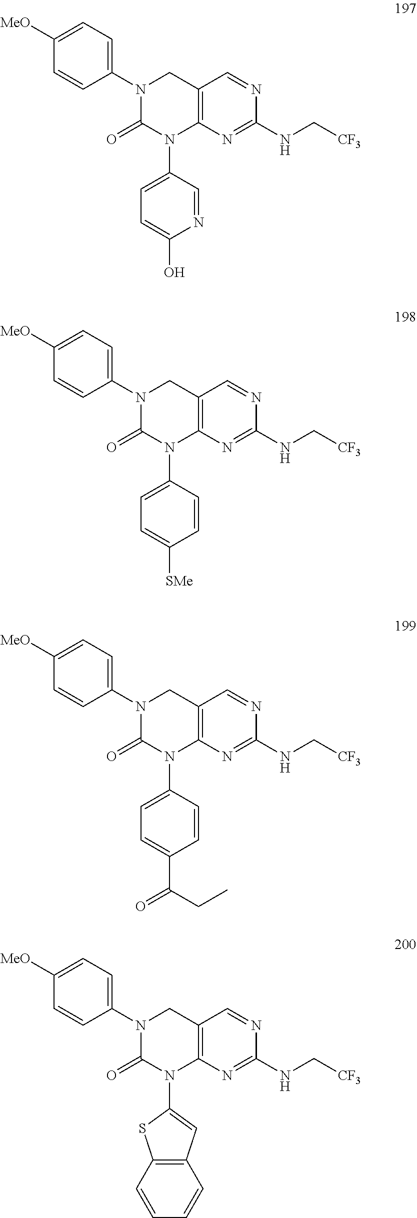 [SMILES: COc1ccc(N2Cc3cnc(NCC(F)(F)F)nc3N(c3ccc(O)nc3)C2=O)cc1 ; COc1ccc(N2Cc3cnc(NCC(F)(F)F)nc3N(c3ccc(SC)cc3)C2=O)cc1 ; CCC(=O)c1ccc(N2C(=O)N(c3ccc(OC)cc3)Cc3cnc(NCC(F)(F)F)nc32)cc1 ; COc1ccc(N2Cc3cnc(NCC(F)(F)F)nc3N(c3cc4ccccc4s3)C2=O)cc1]

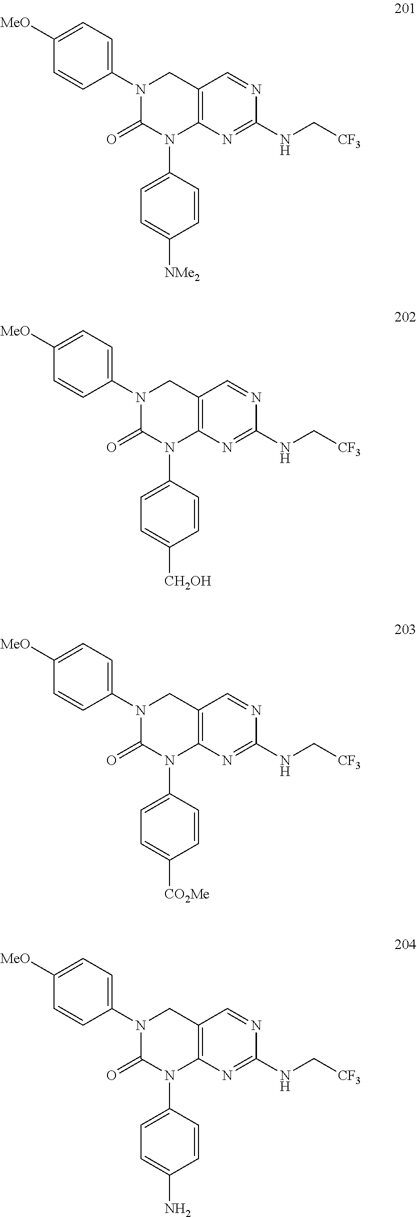 [SMILES: COc1ccc(N2Cc3cnc(NCC(F)(F)F)nc3N(c3ccc(N(C)C)cc3)C2=O)cc1 ; COc1ccc(N2Cc3cnc(NCC(F)(F)F)nc3N(c3ccc(CO)cc3)C2=O)cc1 ; COC(=O)c1ccc(N2C(=O)N(c3ccc(OC)cc3)Cc3cnc(NCC(F)(F)F)nc32)cc1 ; COc1ccc(N2Cc3cnc(NCC(F)(F)F)nc3N(c3ccc(N)cc3)C2=O)cc1]

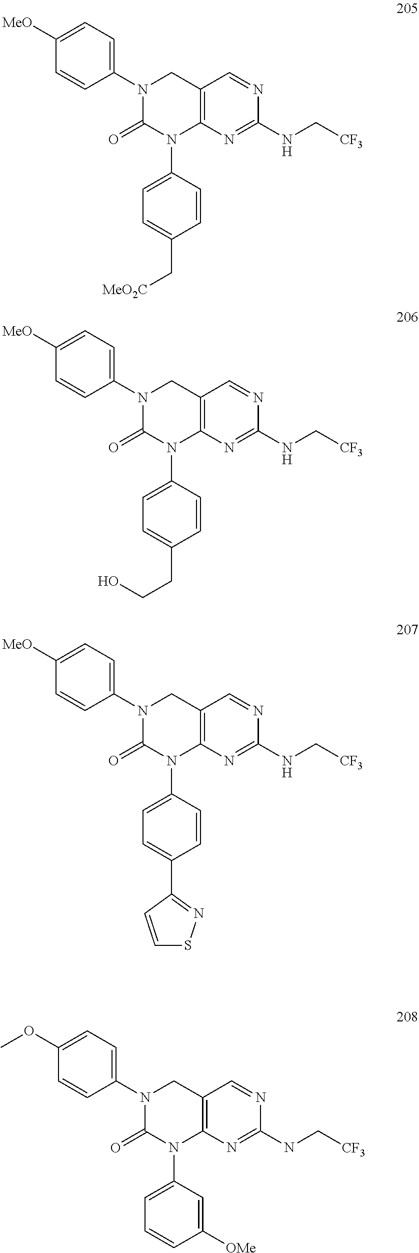 [SMILES: COC(=O)Cc1ccc(N2C(=O)N(c3ccc(OC)cc3)Cc3cnc(NCC(F)(F)F)nc32)cc1 ; COc1ccc(N2Cc3cnc(NCC(F)(F)F)nc3N(c3ccc(CCO)cc3)C2=O)cc1 ; COc1ccc(N2Cc3cnc(NCC(F)(F)F)nc3N(c3ccc(-c4ccsn4)cc3)C2=O)cc1 ; COc1ccc(N2Cc3cnc(NCC(F)(F)F)nc3N(c3cccc(OC)c3)C2=O)cc1]

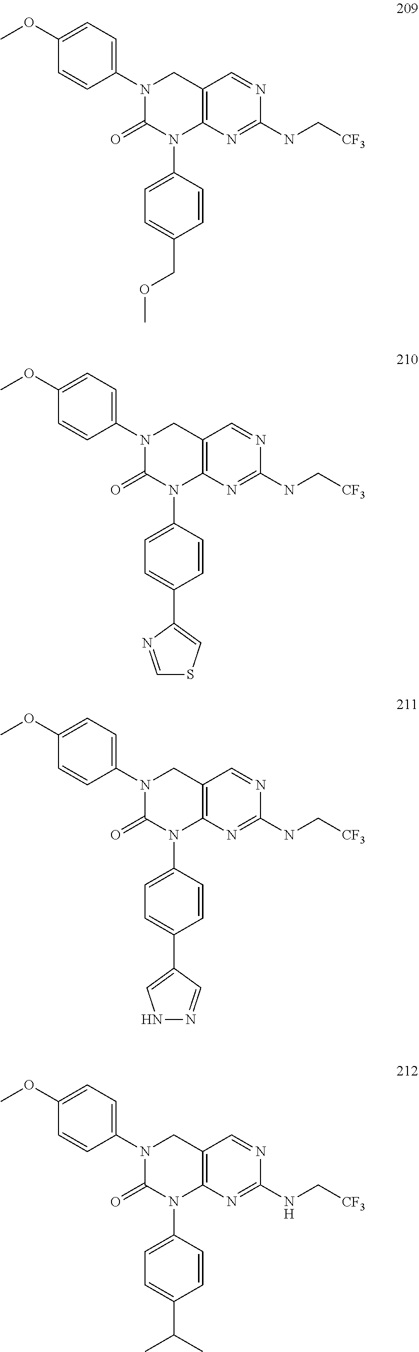 [SMILES: COCc1ccc(N2C(=O)N(c3ccc(OC)cc3)Cc3cnc(NCC(F)(F)F)nc32)cc1 ; COc1ccc(N2Cc3cnc(NCC(F)(F)F)nc3N(c3ccc(-c4cscn4)cc3)C2=O)cc1 ; COc1ccc(N2Cc3cnc(NCC(F)(F)F)nc3N(c3ccc(-c4cn[nH]c4)cc3)C2=O)cc1 ; COc1ccc(N2Cc3cnc(NCC(F)(F)F)nc3N(c3ccc(C(C)C)cc3)C2=O)cc1]

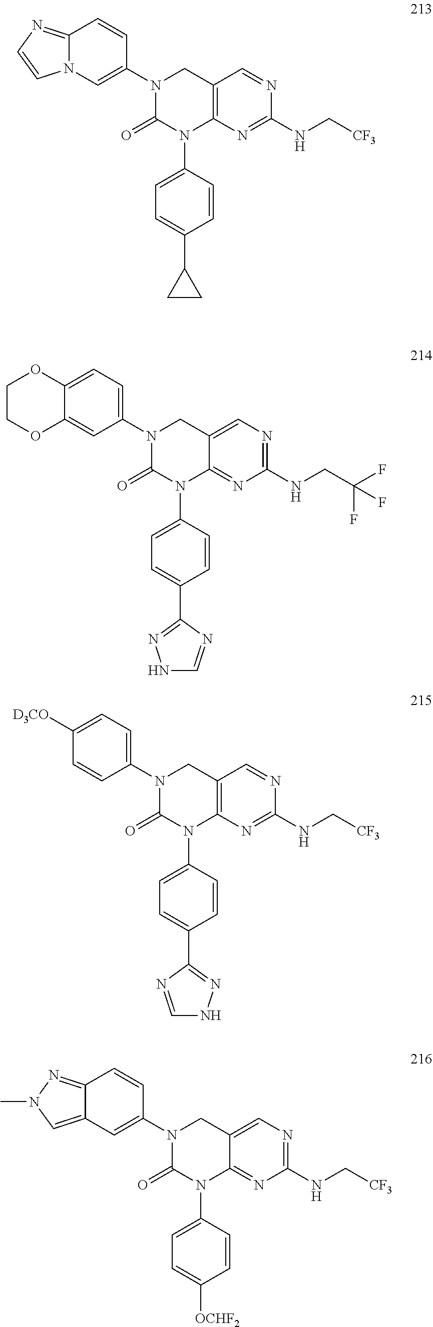

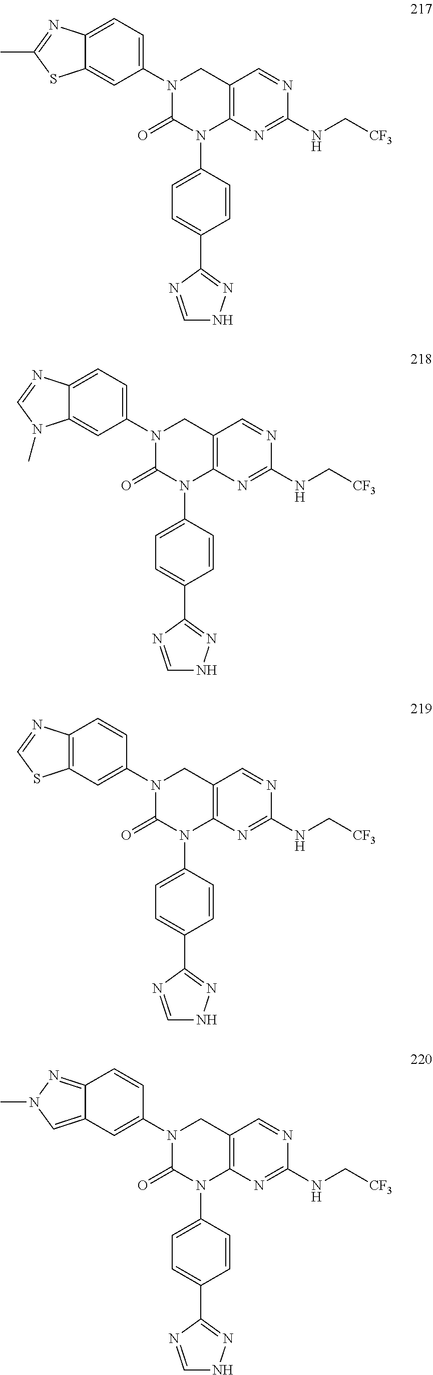 [SMILES: Cc1nc2ccc(N3Cc4cnc(NCC(F)(F)F)nc4N(c4ccc(-c5nc[nH]n5)cc4)C3=O)cc2s1 ; Cn1cnc2ccc(N3Cc4cnc(NCC(F)(F)F)nc4N(c4ccc(-c5nc[nH]n5)cc4)C3=O)cc21 ; O=C1N(c2ccc3ncsc3c2)Cc2cnc(NCC(F)(F)F)nc2N1c1ccc(-c2nc[nH]n2)cc1 ; Cn1cc2cc(N3Cc4cnc(NCC(F)(F)F)nc4N(c4ccc(-c5nc[nH]n5)cc4)C3=O)ccc2n1]

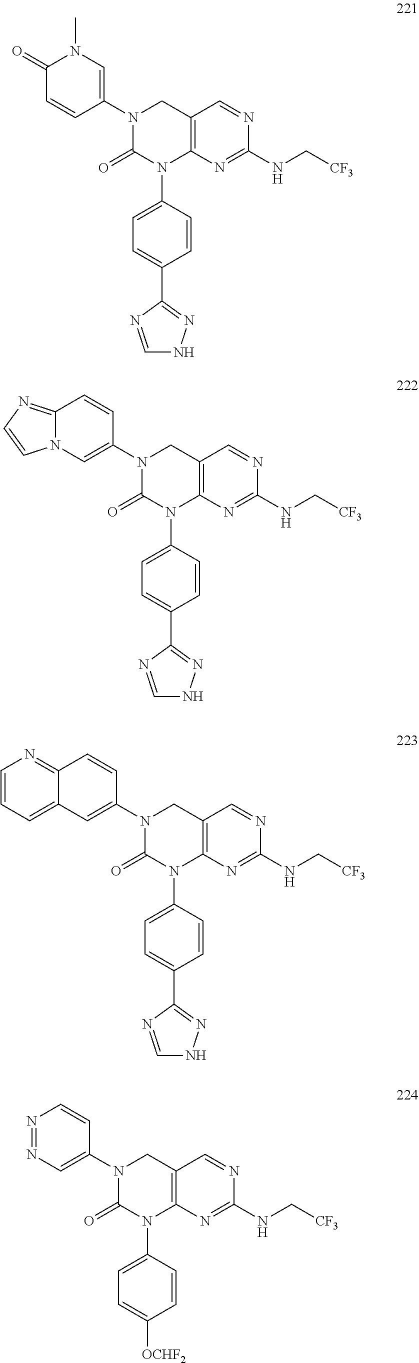 [SMILES: Cn1cc(N2Cc3cnc(NCC(F)(F)F)nc3N(c3ccc(-c4nc[nH]n4)cc3)C2=O)ccc1=O ; O=C1N(c2ccc3nccn3c2)Cc2cnc(NCC(F)(F)F)nc2N1c1ccc(-c2nc[nH]n2)cc1 ; O=C1N(c2ccc3ncccc3c2)Cc2cnc(NCC(F)(F)F)nc2N1c1ccc(-c2nc[nH]n2)cc1 ; O=C1N(c2ccnnc2)Cc2cnc(NCC(F)(F)F)nc2N1c1ccc(OC(F)F)cc1]

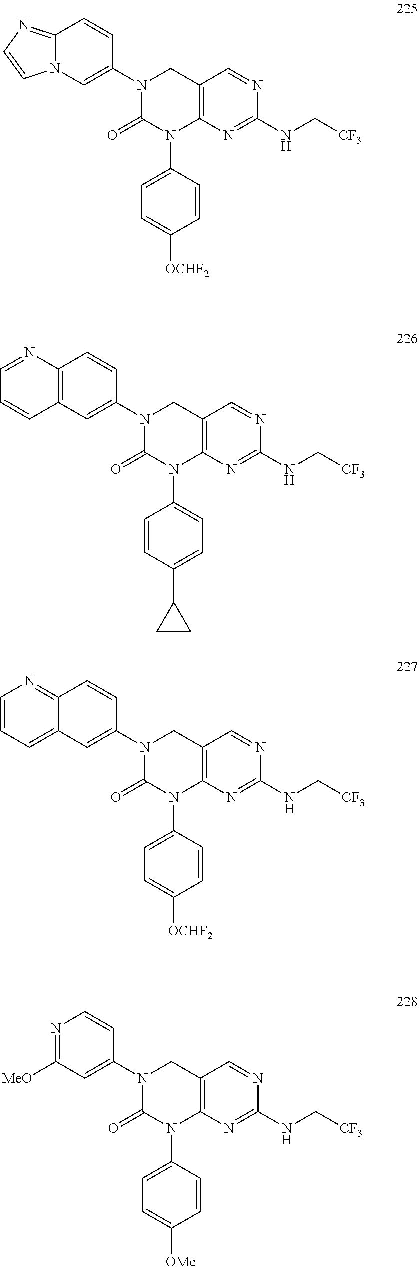 [SMILES: O=C1N(c2ccc3nccn3c2)Cc2cnc(NCC(F)(F)F)nc2N1c1ccc(OC(F)F)cc1 ; O=C1N(c2ccc3ncccc3c2)Cc2cnc(NCC(F)(F)F)nc2N1c1ccc(C2CC2)cc1 ; O=C1N(c2ccc3ncccc3c2)Cc2cnc(NCC(F)(F)F)nc2N1c1ccc(OC(F)F)cc1 ; COc1ccc(N2C(=O)N(c3ccnc(OC)c3)Cc3cnc(NCC(F)(F)F)nc32)cc1]

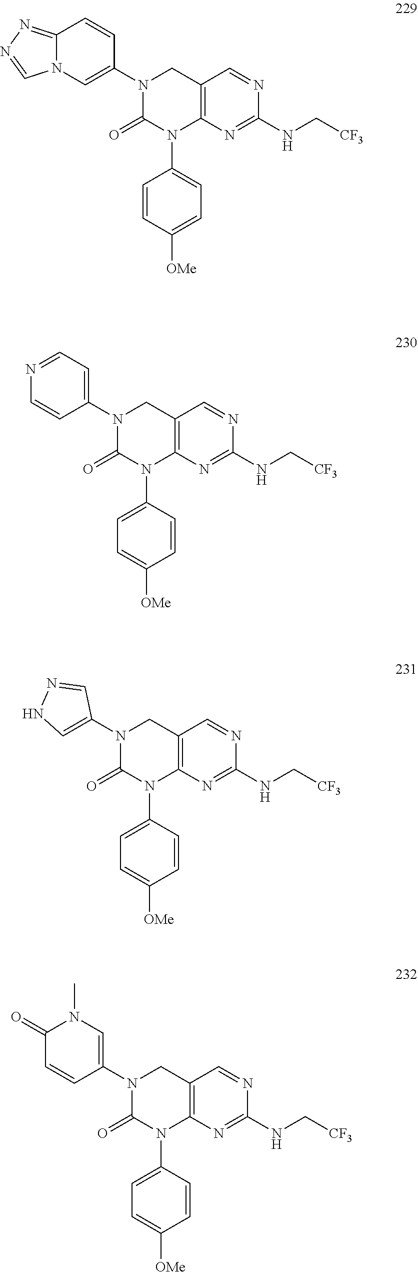 [SMILES: COc1ccc(N2C(=O)N(c3ccc4nncn4c3)Cc3cnc(NCC(F)(F)F)nc32)cc1 ; COc1ccc(N2C(=O)N(c3ccncc3)Cc3cnc(NCC(F)(F)F)nc32)cc1 ; COc1ccc(N2C(=O)N(c3cn[nH]c3)Cc3cnc(NCC(F)(F)F)nc32)cc1 ; COc1ccc(N2C(=O)N(c3ccc(=O)n(C)c3)Cc3cnc(NCC(F)(F)F)nc32)cc1]

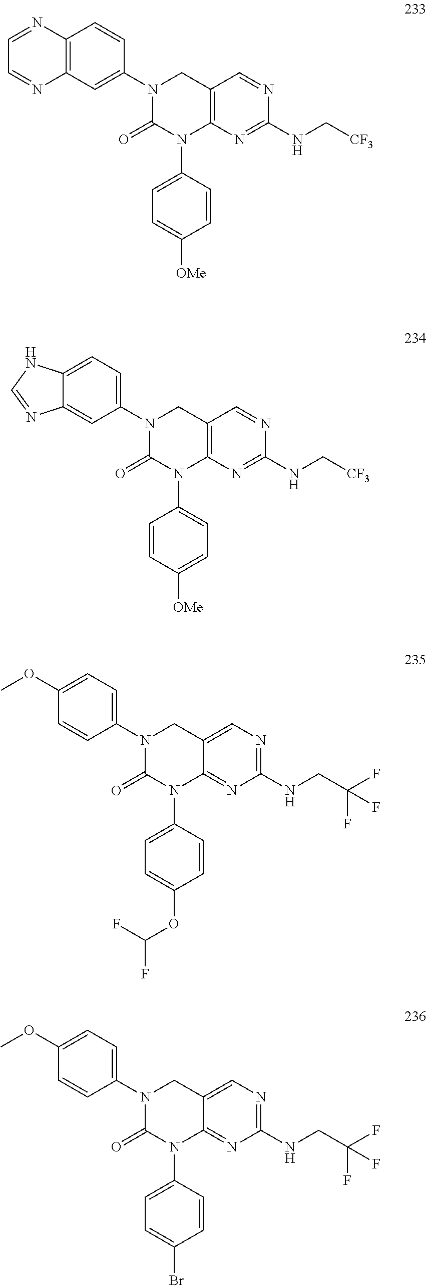 [SMILES: COc1ccc(N2C(=O)N(c3ccc4nccnc4c3)Cc3cnc(NCC(F)(F)F)nc32)cc1 ; COc1ccc(N2C(=O)N(c3ccc4[nH]cnc4c3)Cc3cnc(NCC(F)(F)F)nc32)cc1 ; COc1ccc(N2Cc3cnc(NCC(F)(F)F)nc3N(c3ccc(OC(F)F)cc3)C2=O)cc1 ; COc1ccc(N2Cc3cnc(NCC(F)(F)F)nc3N(c3ccc(Br)cc3)C2=O)cc1]

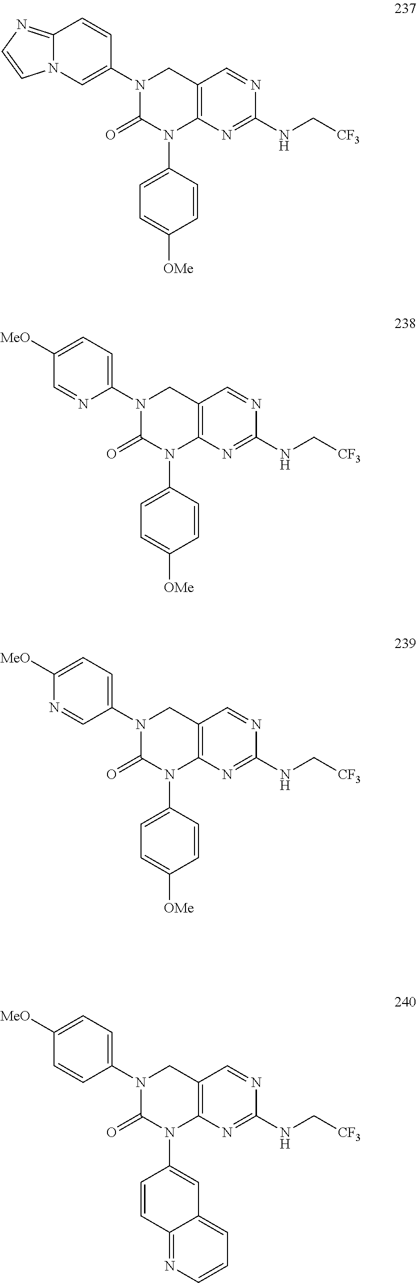 [SMILES: COc1ccc(N2C(=O)N(c3ccc4nccn4c3)Cc3cnc(NCC(F)(F)F)nc32)cc1 ; COc1ccc(N2C(=O)N(c3ccc(OC)cn3)Cc3cnc(NCC(F)(F)F)nc32)cc1 ; COc1ccc(N2C(=O)N(c3ccc(OC)nc3)Cc3cnc(NCC(F)(F)F)nc32)cc1 ; COc1ccc(N2Cc3cnc(NCC(F)(F)F)nc3N(c3ccc4ncccc4c3)C2=O)cc1]

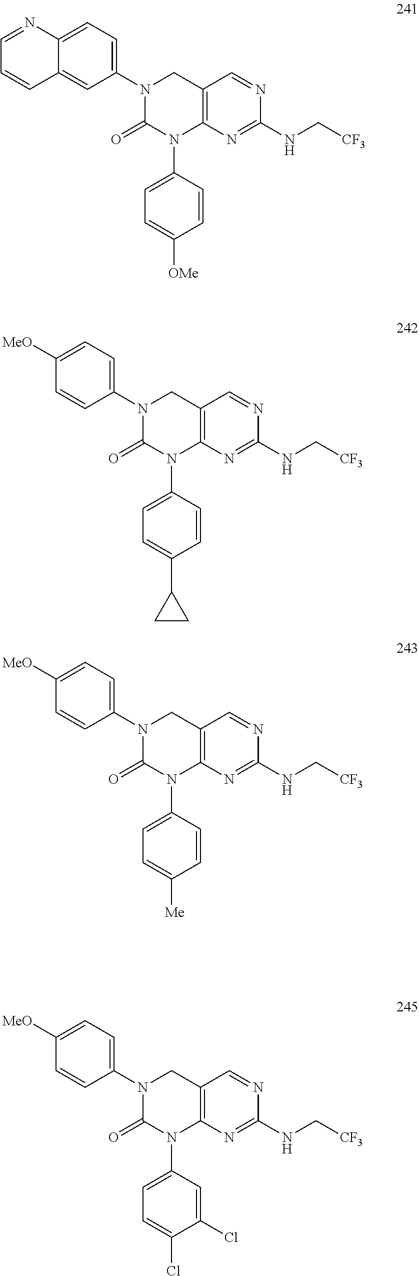 [SMILES: COc1ccc(N2C(=O)N(c3ccc4ncccc4c3)Cc3cnc(NCC(F)(F)F)nc32)cc1 ; COc1ccc(N2Cc3cnc(NCC(F)(F)F)nc3N(c3ccc(C4CC4)cc3)C2=O)cc1 ; COc1ccc(N2Cc3cnc(NCC(F)(F)F)nc3N(c3ccc(C)cc3)C2=O)cc1 ; COc1ccc(N2Cc3cnc(NCC(F)(F)F)nc3N(c3ccc(Cl)c(Cl)c3)C2=O)cc1]

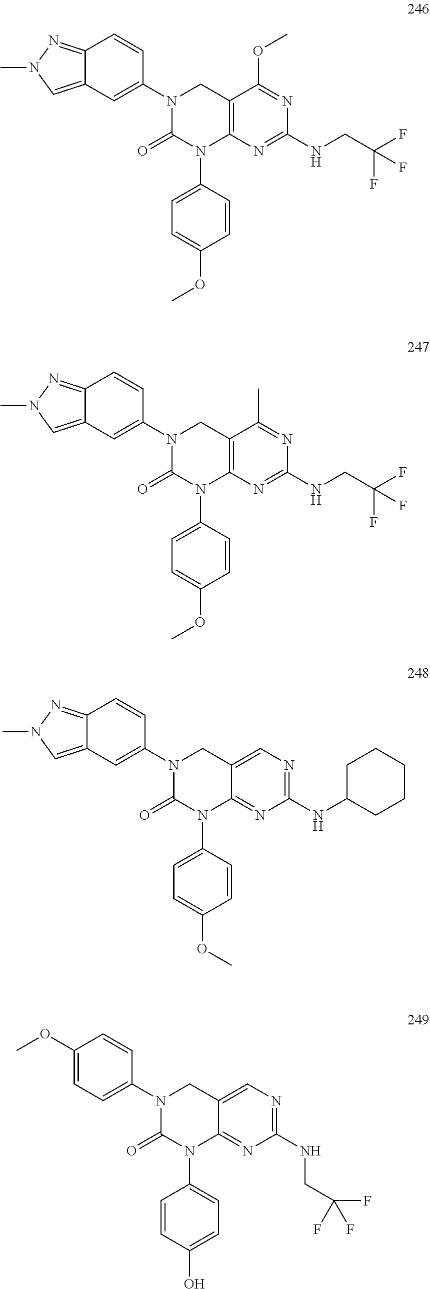 [SMILES: COc1ccc(N2C(=O)N(c3ccc4nn(C)cc4c3)Cc3c(OC)nc(NCC(F)(F)F)nc32)cc1 ; COc1ccc(N2C(=O)N(c3ccc4nn(C)cc4c3)Cc3c(C)nc(NCC(F)(F)F)nc32)cc1 ; COc1ccc(N2C(=O)N(c3ccc4nn(C)cc4c3)Cc3cnc(NC4CCCCC4)nc32)cc1 ; COc1ccc(N2Cc3cnc(NCC(F)(F)F)nc3N(c3ccc(O)cc3)C2=O)cc1]

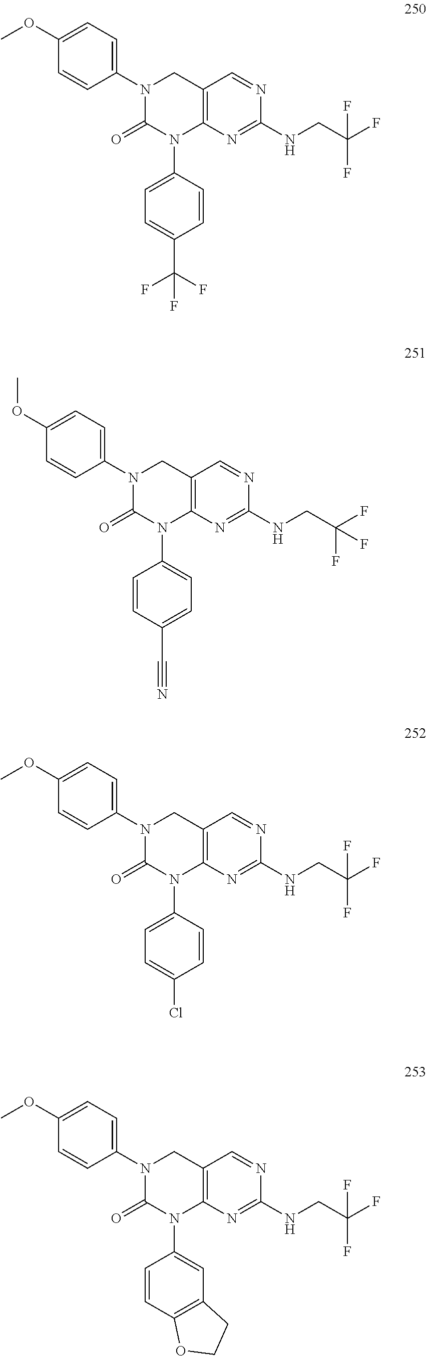 [SMILES: COc1ccc(N2Cc3cnc(NCC(F)(F)F)nc3N(c3ccc(C(F)(F)F)cc3)C2=O)cc1 ; COc1ccc(N2Cc3cnc(NCC(F)(F)F)nc3N(c3ccc(C#N)cc3)C2=O)cc1 ; COc1ccc(N2Cc3cnc(NCC(F)(F)F)nc3N(c3ccc(Cl)cc3)C2=O)cc1 ; COc1ccc(N2Cc3cnc(NCC(F)(F)F)nc3N(c3ccc4c(c3)CCO4)C2=O)cc1]

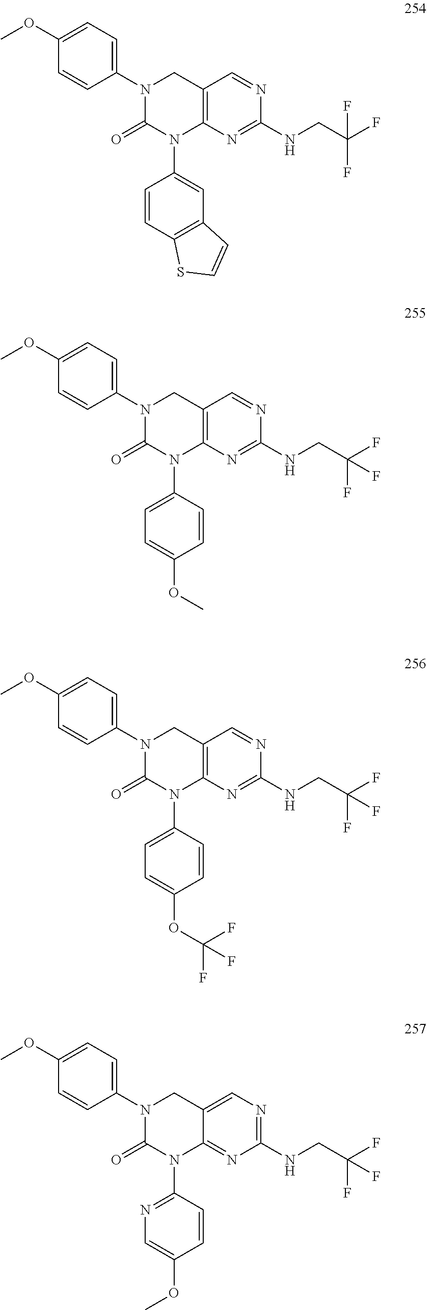 [SMILES: COc1ccc(N2Cc3cnc(NCC(F)(F)F)nc3N(c3ccc4sccc4c3)C2=O)cc1 ; COc1ccc(N2Cc3cnc(NCC(F)(F)F)nc3N(c3ccc(OC)cc3)C2=O)cc1 ; COc1ccc(N2Cc3cnc(NCC(F)(F)F)nc3N(c3ccc(OC(F)(F)F)cc3)C2=O)cc1 ; COc1ccc(N2Cc3cnc(NCC(F)(F)F)nc3N(c3ccc(OC)cn3)C2=O)cc1]

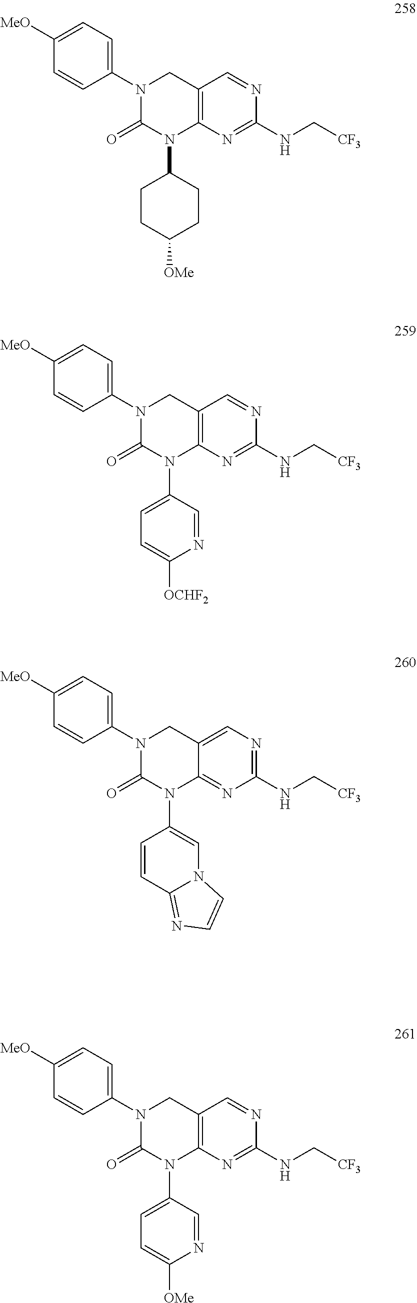

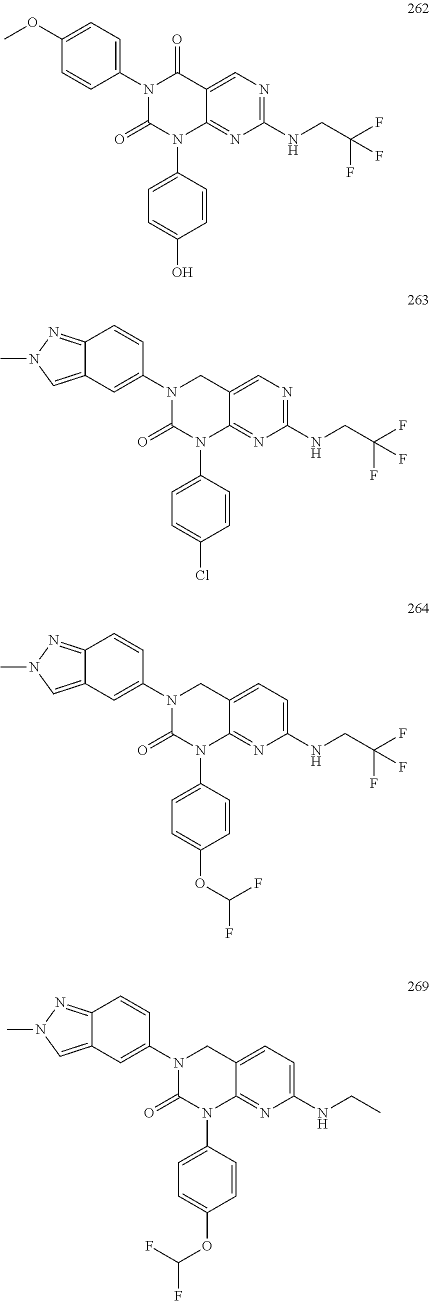 [SMILES: COc1ccc(-n2c(=O)c3cnc(NCC(F)(F)F)nc3n(-c3ccc(O)cc3)c2=O)cc1 ; Cn1cc2cc(N3Cc4cnc(NCC(F)(F)F)nc4N(c4ccc(Cl)cc4)C3=O)ccc2n1 ; Cn1cc2cc(N3Cc4ccc(NCC(F)(F)F)nc4N(c4ccc(OC(F)F)cc4)C3=O)ccc2n1 ; CCNc1ccc2c(n1)N(c1ccc(OC(F)F)cc1)C(=O)N(c1ccc3nn(C)cc3c1)C2]

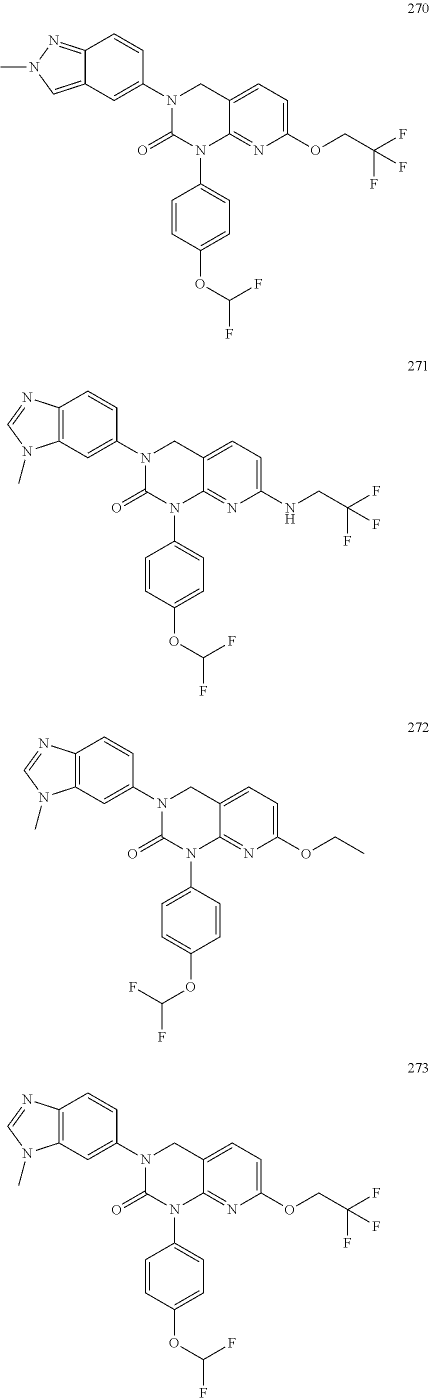 [SMILES: Cn1cc2cc(N3Cc4ccc(OCC(F)(F)F)nc4N(c4ccc(OC(F)F)cc4)C3=O)ccc2n1 ; Cn1cnc2ccc(N3Cc4ccc(NCC(F)(F)F)nc4N(c4ccc(OC(F)F)cc4)C3=O)cc21 ; CCOc1ccc2c(n1)N(c1ccc(OC(F)F)cc1)C(=O)N(c1ccc3ncn(C)c3c1)C2 ; Cn1cnc2ccc(N3Cc4ccc(OCC(F)(F)F)nc4N(c4ccc(OC(F)F)cc4)C3=O)cc21]

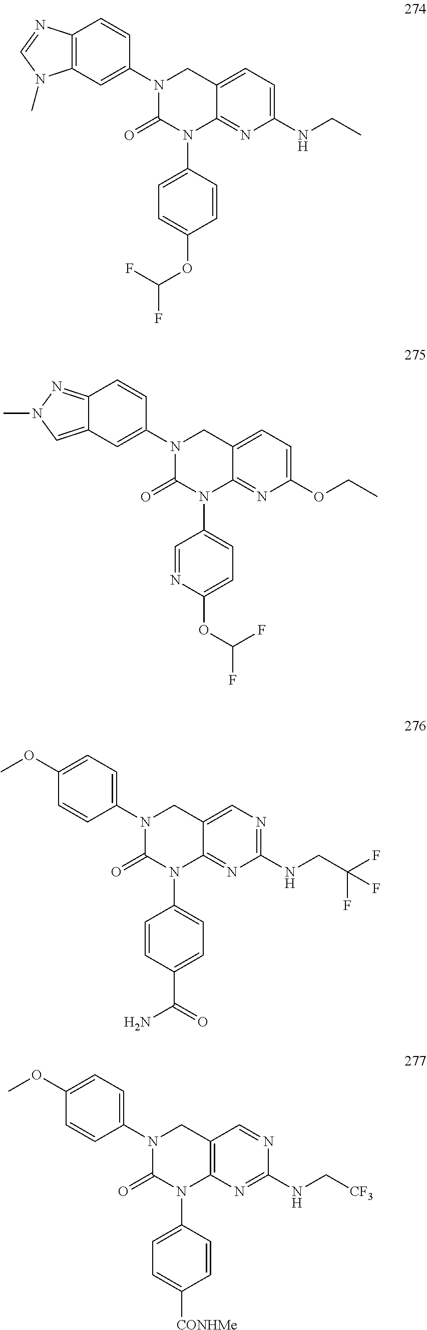

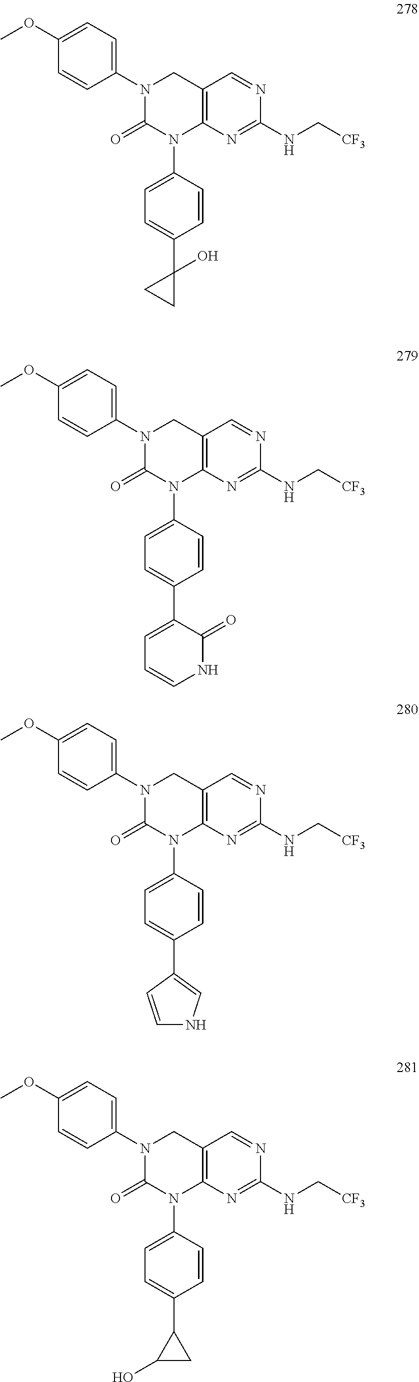 [SMILES: COc1ccc(N2Cc3cnc(NCC(F)(F)F)nc3N(c3ccc(C4(O)CC4)cc3)C2=O)cc1 ; COc1ccc(N2Cc3cnc(NCC(F)(F)F)nc3N(c3ccc(-c4ccc[nH]c4=O)cc3)C2=O)cc1 ; COc1ccc(N2Cc3cnc(NCC(F)(F)F)nc3N(c3ccc(-c4cc[nH]c4)cc3)C2=O)cc1 ; COc1ccc(N2Cc3cnc(NCC(F)(F)F)nc3N(c3ccc(C4CC4O)cc3)C2=O)cc1]

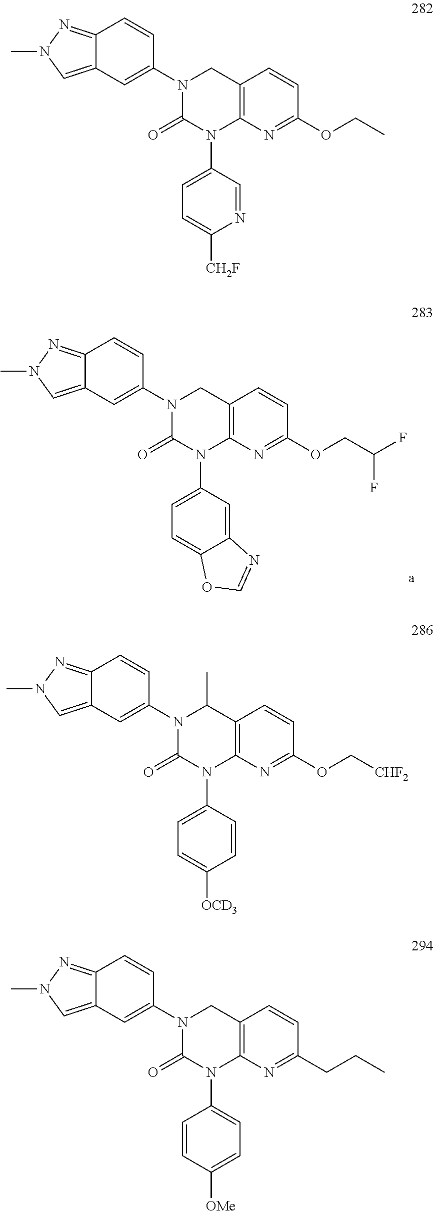

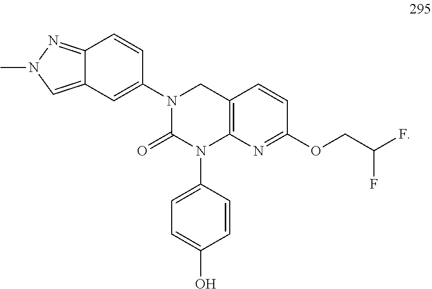 [SMILES: Cn1cc2cc(N3Cc4ccc(OCC(F)F)nc4N(c4ccc(O)cc4)C3=O)ccc2n1]

36. The compound according to claim 2 or a pharmaceutically acceptable salt thereof, wherein the compound is:

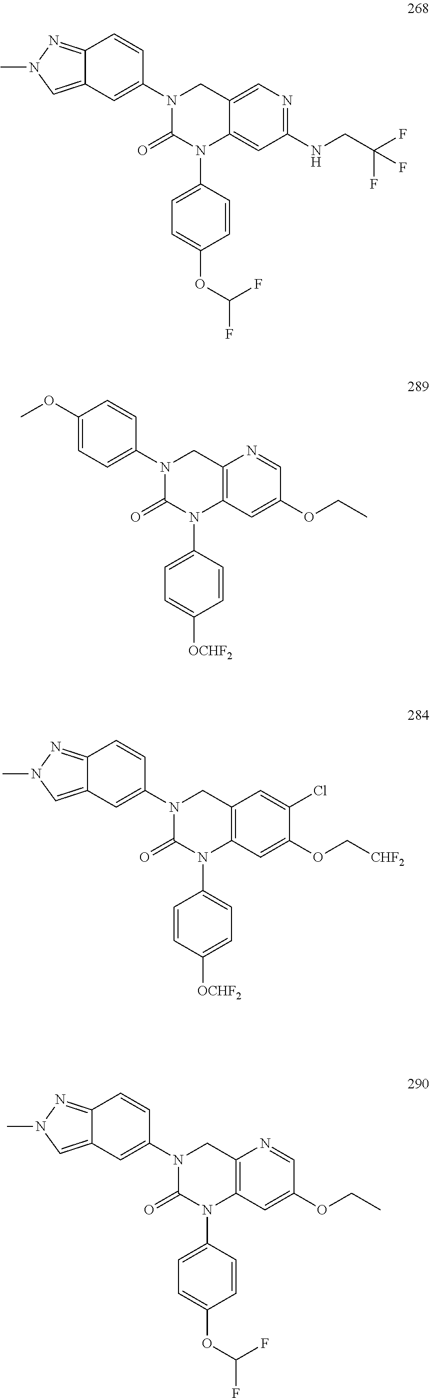

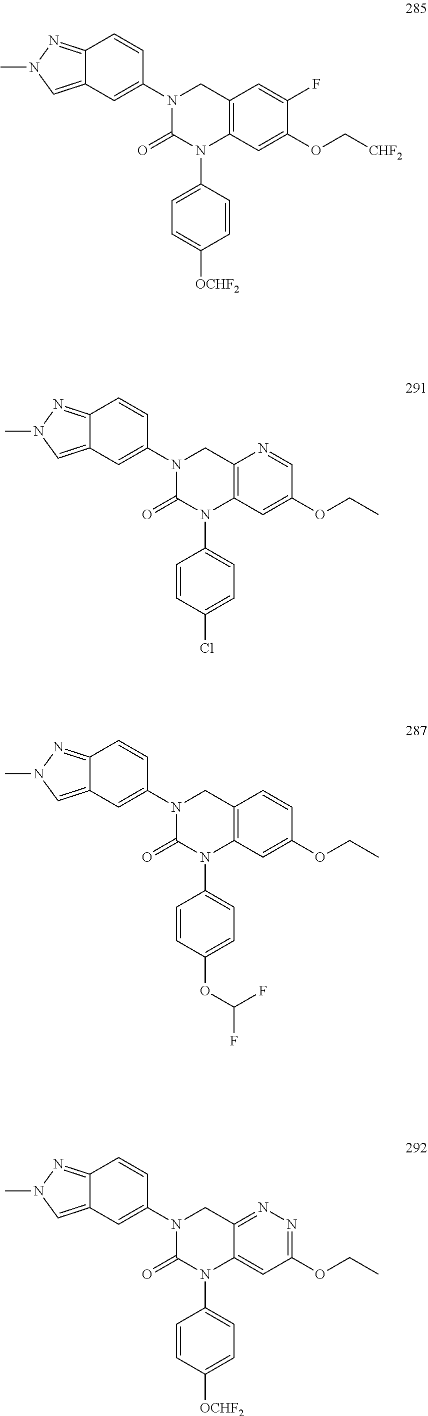

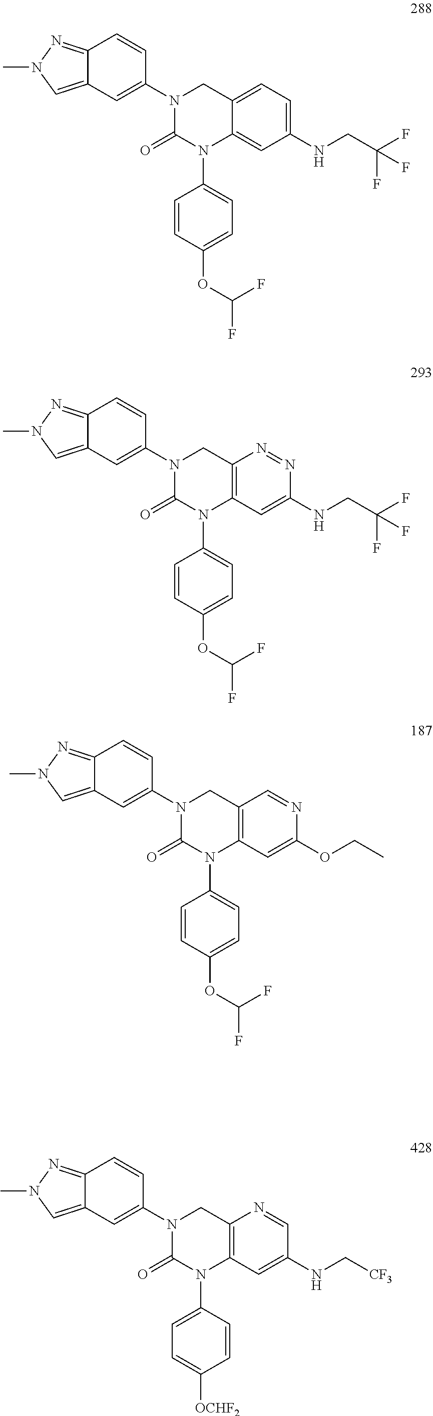

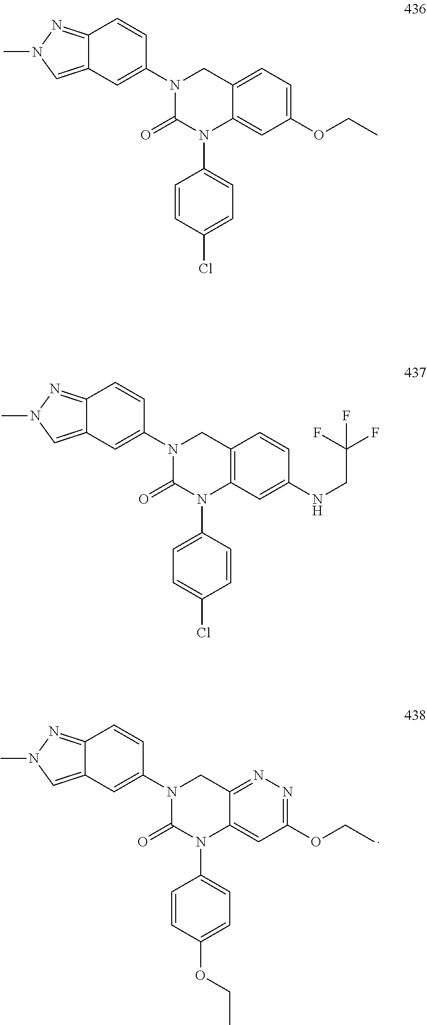

37. The compound according to claim 1 or a pharmaceutically acceptable salt thereof, wherein the compound is:

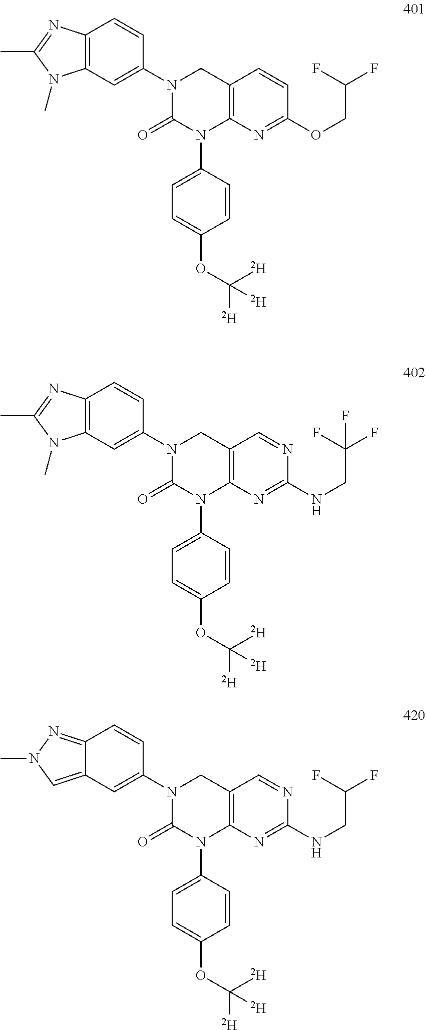 [SMILES: [2H]C([2H])([2H])Oc1ccc(N2C(=O)N(c3ccc4nc(C)n(C)c4c3)Cc3ccc(OCC(F)F)nc32)cc1 ; [2H]C([2H])([2H])Oc1ccc(N2C(=O)N(c3ccc4nc(C)n(C)c4c3)Cc3cnc(NCC(F)(F)F)nc32)cc1 ; [2H]C([2H])([2H])Oc1ccc(N2C(=O)N(c3ccc4nn(C)cc4c3)Cc3cnc(NCC(F)F)nc32)cc1]

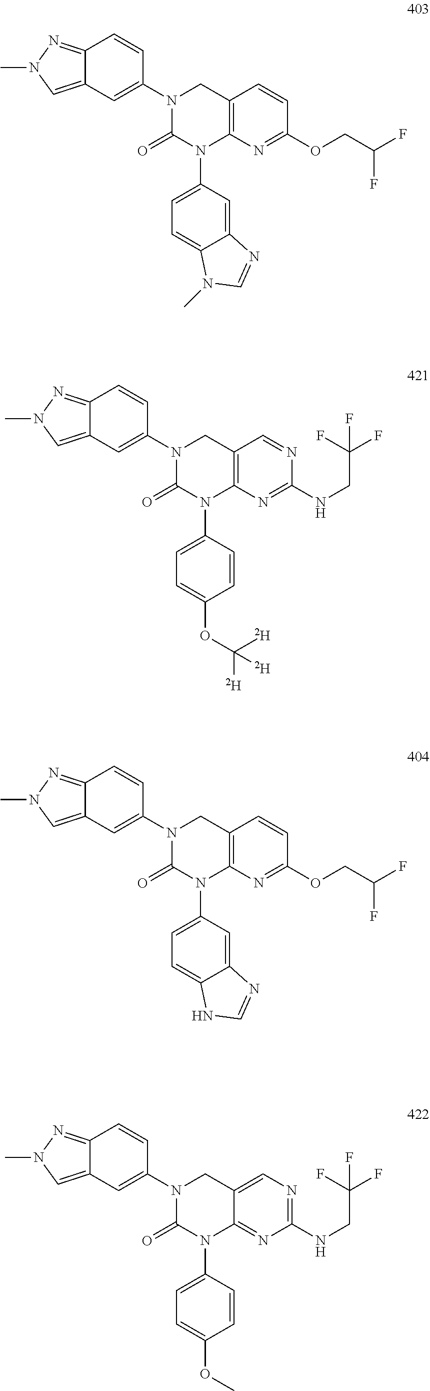 [SMILES: Cn1cc2cc(N3Cc4ccc(OCC(F)F)nc4N(c4ccc5c(c4)ncn5C)C3=O)ccc2n1 ; [2H]C([2H])([2H])Oc1ccc(N2C(=O)N(c3ccc4nn(C)cc4c3)Cc3cnc(NCC(F)(F)F)nc32)cc1 ; Cn1cc2cc(N3Cc4ccc(OCC(F)F)nc4N(c4ccc5[nH]cnc5c4)C3=O)ccc2n1 ; COc1ccc(N2C(=O)N(c3ccc4nn(C)cc4c3)Cc3cnc(NCC(F)(F)F)nc32)cc1]

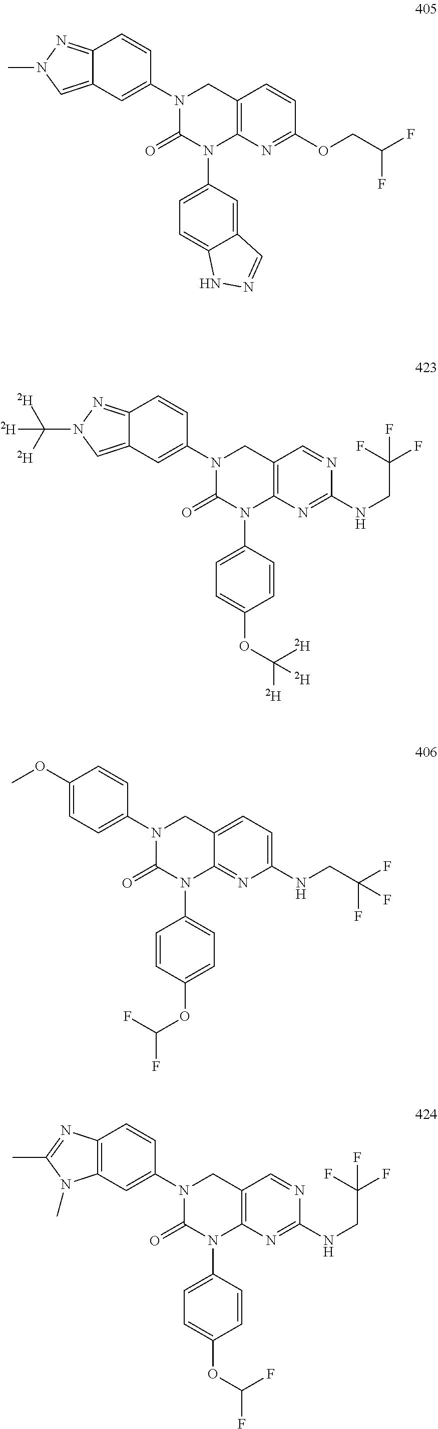 [SMILES: Cn1cc2cc(N3Cc4ccc(OCC(F)F)nc4N(c4ccc5[nH]ncc5c4)C3=O)ccc2n1 ; [2H]C([2H])([2H])Oc1ccc(N2C(=O)N(c3ccc4nn(C([2H])([2H])[2H])cc4c3)Cc3cnc(NCC(F)(F)F)nc32)cc1 ; COc1ccc(N2Cc3ccc(NCC(F)(F)F)nc3N(c3ccc(OC(F)F)cc3)C2=O)cc1 ; Cc1nc2ccc(N3Cc4cnc(NCC(F)(F)F)nc4N(c4ccc(OC(F)F)cc4)C3=O)cc2n1C]

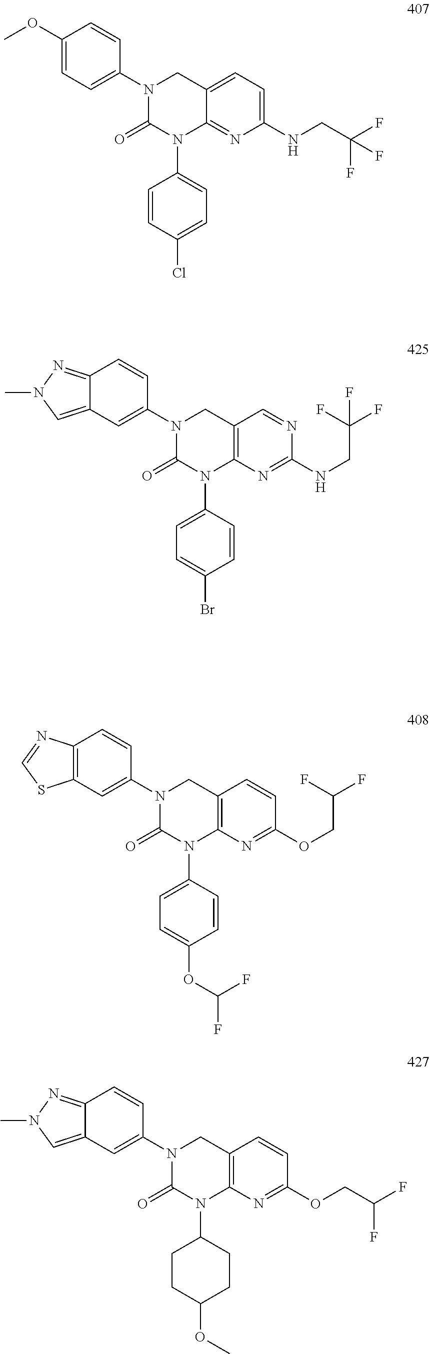 [SMILES: COc1ccc(N2Cc3ccc(NCC(F)(F)F)nc3N(c3ccc(Cl)cc3)C2=O)cc1 ; Cn1cc2cc(N3Cc4cnc(NCC(F)(F)F)nc4N(c4ccc(Br)cc4)C3=O)ccc2n1 ; O=C1N(c2ccc3ncsc3c2)Cc2ccc(OCC(F)F)nc2N1c1ccc(OC(F)F)cc1 ; COC1CCC(N2C(=O)N(c3ccc4nn(C)cc4c3)Cc3ccc(OCC(F)F)nc32)CC1]

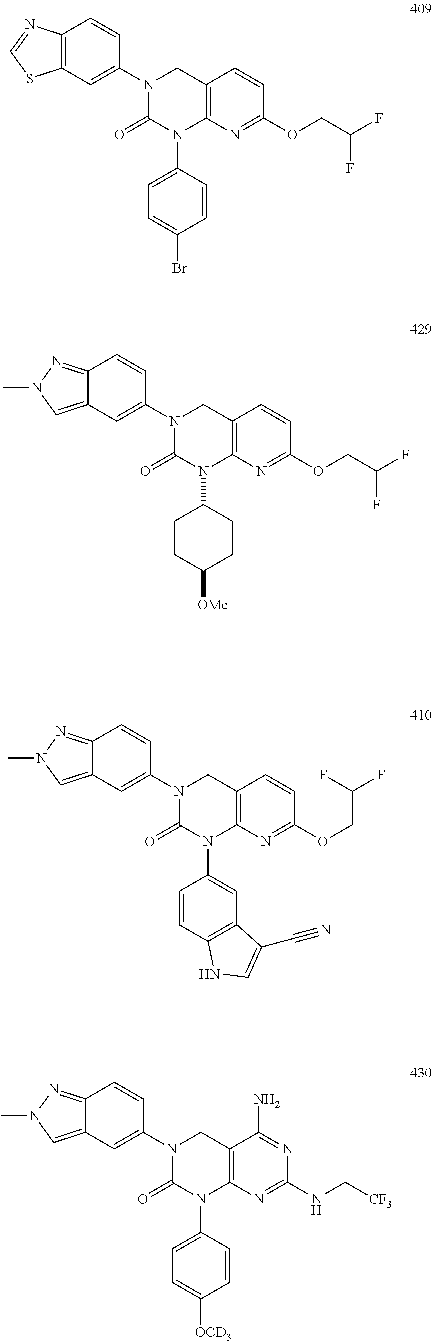

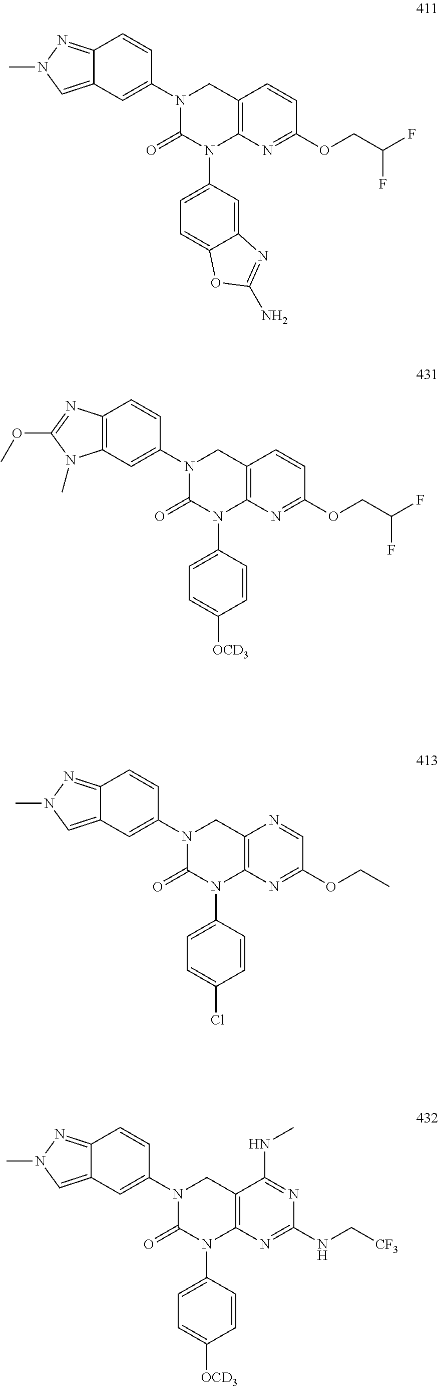

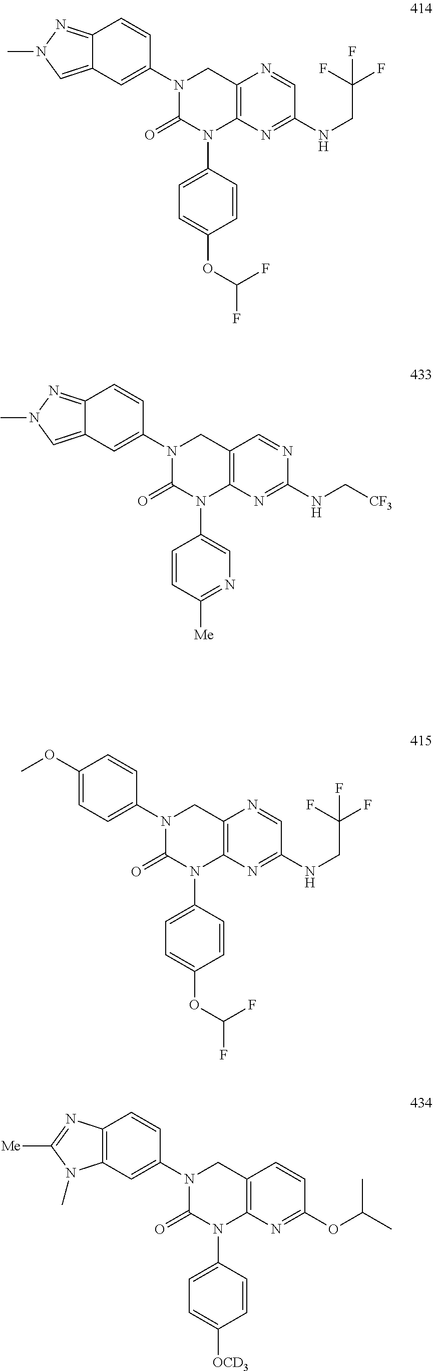

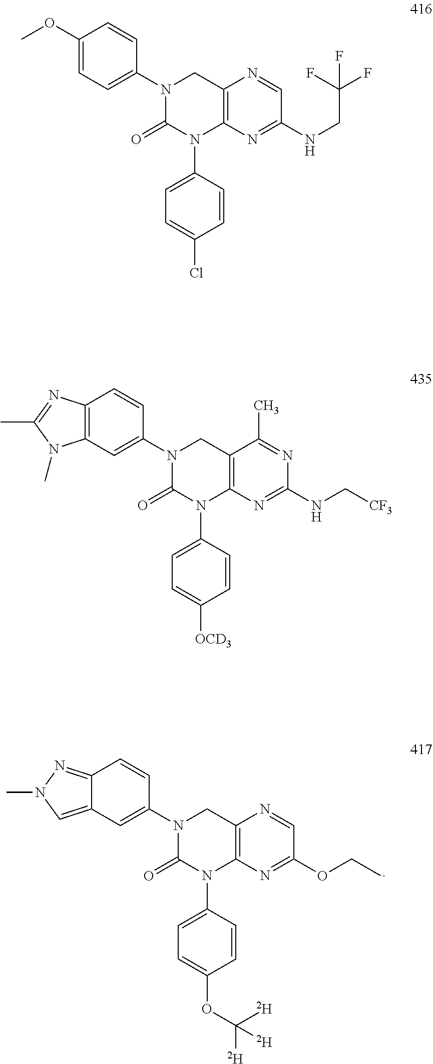

38. The compound according to claim 5 or a pharmaceutically acceptable salt thereof, wherein the compound is:

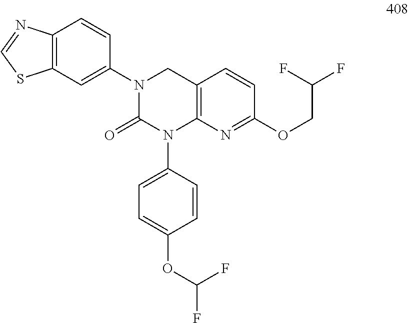 [SMILES: O=C1N(c2ccc3ncsc3c2)Cc2ccc(OCC(F)F)nc2N1c1ccc(OC(F)F)cc1]

39. A pharmaceutical composition comprising a therapeutically effective amount of a compound according to claim 1, or a pharmaceutically acceptable salt thereof, and a pharmaceutically acceptable carrier.

40. A method for treating a cancer in a subject suffering therefrom, comprising administering to the subject an effective amount of a MAT2A inhibitor compound, or a pharmaceutically acceptable salt thereof, according to claim 1.

41. The method according to claim 40, wherein the cancer is an MTAP-deleted cancer.

42. The method according to claim 40, wherein the cancer is selected from the group consisting of mesothelioma, neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, bladder carcinoma, testis carcinoma, breast carcinoma, urinary carcinoma, melanoma, brain tumors, lymphoma, head and neck cancer, acute lymphatic leukemia (ALL), chronic lymphatic leukemia (CLL), acute myeloid leukemia (AML), chronic myeloid leukemia (CML), hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, osteosarcoma, chondrosarcoma, myosarcoma, liposarcoma, fibrosarcoma, Ewing sarcoma, and plasmocytoma.

43. The method according to claim 40, wherein the cancer is selected from the group consisting of B-cell acute lymphocytic leukemia (B-ALL), mesothelioma, lymphoma, pancreatic carcinoma, lung cancer, gastric cancer, esophageal cancer, bladder carcinoma, brain cancer, head and neck cancer, melanoma, and breast cancer.

44. The method according to claim 43, wherein the cancer is a lung cancer is selected from the group consisting of non-small cell lung cancer, small cell lung cancer, adenocarcinoma of the lung, and squamous cell carcinoma of the lung.

45. The method according to claim 43, wherein the cancer is a brain tumor selected from the group consisting of glioma, glioblastoma, astrocytoma, meningioma, medulloblastoma, peripheral neuroectodermal tumors, and craniopharyngioma.

46. The method according to claim 43, wherein the cancer is triple negative breast cancer (TNBC).

47. The method according to claim 43, wherein the cancer is a lymphoma selected from the group consisting of mantle cell lymphoma, Hodgkin lymphoma, non-Hodgkin lymphoma, Burkitt lymphoma, diffuse large B-cell lymphoma, and adult T-cell leukemia/lymphoma.

48. A method for treating a cancer in a subject suffering therefrom, wherein the cancer is characterized by a reduction or absence of methylthioadenosine phosphorylase (MTAP) gene expression, the absence of the MTAP gene, or reduced function of MTAP protein, as compared to cancers where the MTAP gene or protein is present and/or fully functioning, the method comprising administering to the subject a therapeutically effective amount of a compound, or a pharmaceutically acceptable salt thereof, according to claim 1.

49-64. (canceled)

65. A compound that is: 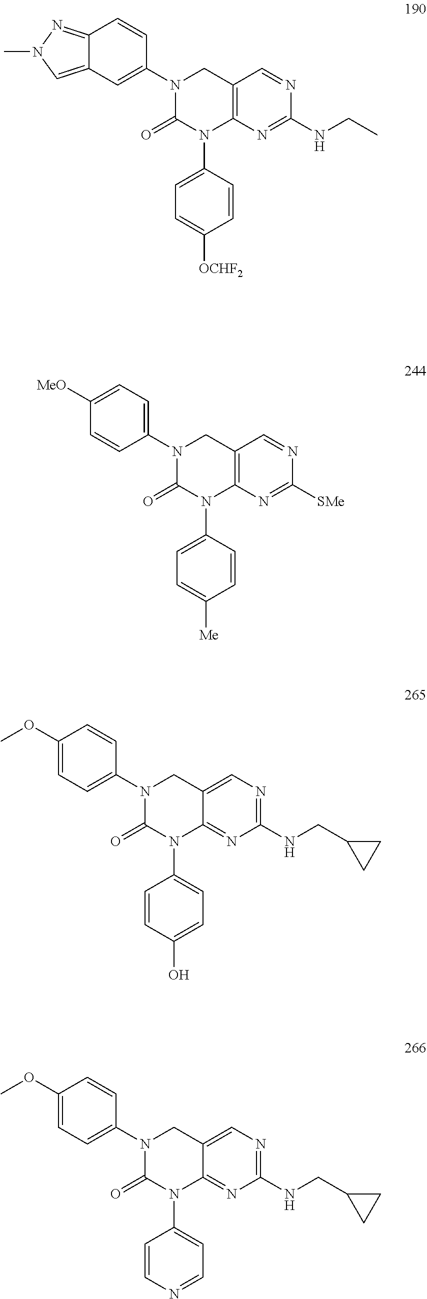 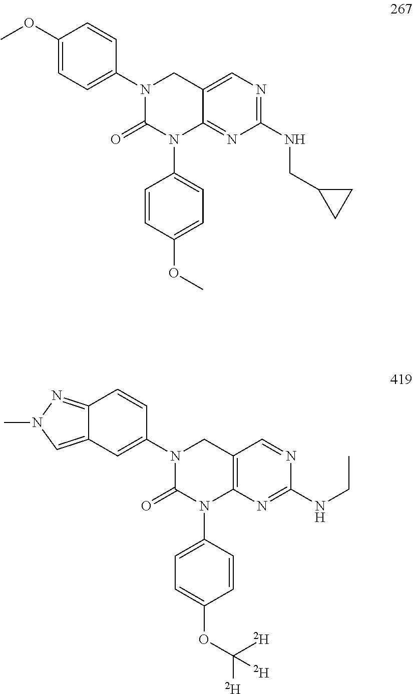 [SMILES: COc1ccc(N2Cc3cnc(NCC4CC4)nc3N(c3ccc(OC)cc3)C2=O)cc1 ; [2H]C([2H])([2H])Oc1ccc(N2C(=O)N(c3ccc4nn(C)cc4c3)Cc3cnc(NCC)nc32)cc1]
or a pharmaceutically acceptable salt thereof.
